(19) United States
(12) Patent Application Publication
(10) Pub. No.: US 2024/0090324 A1
(43) Pub. Date: Mar. 14, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hyo-Jung LEE, Gyeonggi-do (KR); Yea-Mi SONG, Gyeonggi-do (KR); Hyun-Ju KANG, Gyeonggi-do (KR); Mi-Ja LEE, Gyeonggi-do (KR); Doo-Hyeon MOON, Gyeonggi-do (KR); Kyoung-Jin PARK, Gyeonggi-do (KR); Du-Yong PARK, Gyeonggi-do (KR); DaiKyu KIM, Gyeonggi-do (KR)

(21) Appl. No.: 18/354,235

(22) Filed: Jul. 18, 2023

(30) Foreign Application Priority Data
Aug. 4, 2022 (KR) 10-2022-0097272
Jul. 4, 2023 (KR) 10-2023-0086651

(51) Int. Cl. H10K 85/60; C07D 251/24; C09K 11/06

(52) U.S. Cl. CPC H10K 85/654; C07D 251/24; C09K 11/06; H10K 85/615; H10K 50/11

(57) ABSTRACT

The present disclosure relates to a plurality of host materials, an organic electroluminescent compound, and an organic electroluminescent device comprising the same. An organic electroluminescent device having improved driving voltage, luminous efficiency, and/or lifespan properties compared to a conventional organic electroluminescent device can be provided by including the specific combination of compounds according to the present disclosure as a plurality of host materials or by including the compound according to the present disclosure.

### TECHNICAL FIELD

The present disclosure relates to a plurality of host materials, an organic electroluminescent compound, and an organic electroluminescent device comprising the same.

### BACKGROUND ART

In 1987, Tang et al. of Eastman Kodak first developed a small molecular green organic electroluminescent device (OLED) by using TPD/Alq₃ bilayer consisting of a light-emitting layer and a charge transport layer. Thereafter, the development of OLEDs was rapidly effected and OLEDs have been commercialized. Currently, OLEDs mainly use phosphorescent materials having excellent luminous efficiency in panel implementation. However, in many applications such as TV and lighting, the lifespan of OLEDs is insufficient, and higher efficiency of OLEDs is still required. In general, the lifespan of an OLED becomes shorter as the luminance of the OLED becomes higher. Thus, OLEDs having high luminous efficiency and/or long lifespan are required for long-term use and high resolution of a display.

In order to improve luminous efficiency, driving voltage, and/or lifespan, various materials or concepts for an organic layer of an organic electroluminescent device have been proposed. However, they were not satisfied in practical use. In addition, it has been continuously required to develop an organic electroluminescent material having more improved performance, such as improved driving voltage, luminous efficiency, power efficiency, and/or lifespan properties, compared to specific combination of compounds previously disclosed.

Meanwhile, Korean Patent Application Laid-Open Nos. 2015-0117173 and 2021-0089596 disclose an organic electroluminescent device comprising a compound having an aryl-substituted pyrimidinyl and/or triazinyl structure as a backbone and a compound having biscarbazole as a backbone, but fail to specifically disclose a plurality of host materials comprising a specific combination of compounds claimed herein. In addition, Korean Patent Application Laid-Open Nos. 2016-0141672 and 2015-0088712 disclose a compound having an aryl-substituted pyrimidinyl and/or triazinyl structure as a backbone, but fail to specifically disclose an organic electroluminescent compound claimed herein. Accordingly, it has been continuously required to develop a light-emitting material having more improved performance, such as improved driving voltage, luminous efficiency, and/or lifespan properties, compared to specific combination of compounds previously disclosed.

### DISCLOSURE OF INVENTION

### Technical Problems

The objective of the present disclosure is to provide improved plurality of host materials capable of providing an organic electroluminescent device with improved driving voltage, luminous efficiency, and/or lifespan properties. Another objective of the present disclosure is to provide an organic electroluminescent compound having a novel structure suitable for application to an organic electroluminescent device. Still another objective of the present disclosure is to provide an organic electroluminescent device with improved driving voltage, luminous efficiency, and/or lifespan properties by including a compound according to the present disclosure or by including a specific combination of compounds according to the present disclosure.

### Solution to Problem

As a result of intensive research to solve the above technical problems, the present inventors found that the above objectives can be achieved by a plurality of host materials comprising a first host compound comprising a compound represented by the following formula 1 and a second host compound comprising a compound represented by the following formula 2, wherein at least one of the first host compound and the second host compound contains deuterium; or by a compound represented by the following formula 1-1.

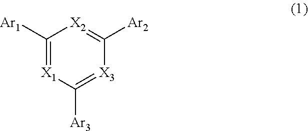

In formula 1,
X₁ to X₃ each independently represent N or CRₐ; with the proviso that at least two of X₁ to X₃ are N;
Rₐ represents hydrogen or deuterium; and
Ar₁ to Ar₃ each independently represent a (C6-C30)aryl unsubstituted or substituted with at least one of deuterium and a (C6-C30)aryl(s);

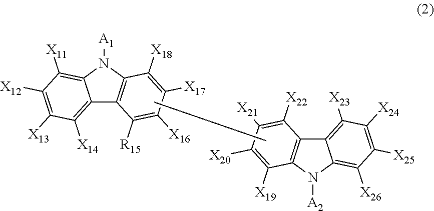

in formula 2,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl;
any one of X₁₅ to X₁₃ and any one of X₁₉ to X₂₂ are linked to each other to form a single bond; and
X₁₁ to X₁₄, X₂₃ to X₂₆, and X₁₅ to X₂₂ which do not form a single bond each independently represent hydrogen, deuterium, a substituted or unsubstituted (C6-C30)aryl, or a substituted or unsubstituted (3- to 30-membered)heteroaryl; or may be linked to an adjacent substituent to form a ring(s).

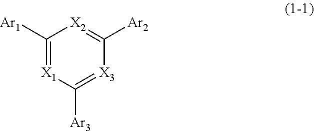

In formula 1-1,
X₁ to X₃ each independently represent N or CRₐ; with the proviso that at least two of X₁ to X₃ are N;
Rₐ represents hydrogen or deuterium; and
Ar₁ to Ar₃ each independently represent a (C6-C30)aryl unsubstituted or substituted with at least one of deuterium and a (C6-C30)aryl(s); with the proviso that Ar₁ to Ar₃ are different from each other, and the structure comprising naphthalene and the following structures are excluded.

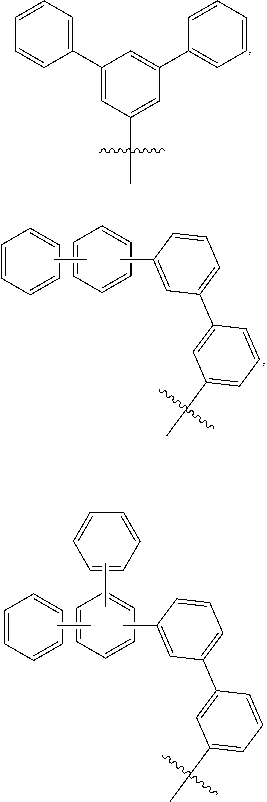

### Advantageous Effects of Invention

An organic electroluminescent device having lower driving voltage, higher luminous efficiency, and/or excellent lifespan properties compared to a conventional organic electroluminescent device is provided by including a specific combination of compounds according to the present disclosure as a plurality of host materials or by including the compound according to the present disclosure, and it is possible to manufacture a display device or a lighting device using the same.

### MODE FOR THE INVENTION

Hereinafter, the present disclosure will be described in detail. However, the following description is intended to explain the present disclosure, and is not meant to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material(s) comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of two or more host materials that may optionally further comprise a conventional material included in an organic electroluminescent material. Two or more compounds included in the plurality of host materials of the present disclosure may be together included in one light-emitting layer or may be respectively included in different light-emitting layers. For example, the two or more host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, preferably the group consisting of O, S, and N. The above heterocycloalkyl may include tetrahydrofuran, pyrrolidine, thiolane, tetrahydropyran, etc. The term “(C6-C30)aryl” is meant to be a monocyclic or fused ring radical derived from an aromatic hydrocarbon having 6 to 30 ring backbone carbon atoms. The above aryl may be partially saturated; and may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopenten-fluoren]yl, spiro[dihydroinden-fluoren]yl, azulenyl, tetramethyldihydrophenanthrenyl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl” means an aryl group having 3 to 30 ring backbone atoms and including at least one, preferably 1 to 4 heteroatom(s) selected from the group consisting of B, N, O, S, Si, and P. The above heteroaryl may be a monocyclic ring or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, naphthoxazolyl, benzofuroquinolyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, “halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2 or positions 2 and 3, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

“Substituted” in the expression “substituted or unsubstituted” means that a hydrogen atom in a certain functional group is replaced with another atom or another functional group (i.e., a substituent), and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent or as substituents in which two heteroaryl substituents are linked. In the present disclosure, the substituent(s) of the substituted aryl, the substituted heteroaryl, the substituted dibenzofuranyl, the substituted dibenzothiophenyl, and the substituted carbazolyl each independently are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30)aliphatic ring(s) and a (C6-C30)aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (5- to 20-membered)heteroaryl unsubstituted or substituted with deuterium; and a (C6-C28)aryl unsubstituted or substituted with at least one of deuterium and a (C6-C18)aryl(s). According to another embodiment of the present disclosure, the substituent(s), each independently, are at least one selected from the group consisting of deuterium; a (5- to 15-membered)heteroaryl unsubstituted or substituted with deuterium; and a (C6-C20)aryl unsubstituted or substituted with at least one of deuterium and a (C6-C12)aryl(s). For example, the substituent(s), each independently, may be deuterium; or may be at least one selected from a phenyl, a naphthyl, a triphenylenyl, a phenanthrenyl unsubstituted or substituted with phenyl(s), a dibenzofuranyl, and a dibenzothiophenyl, which may be further substituted with deuterium.

In the formulas of the present disclosure, when a ring is formed by a linkage of adjacent substituents, the ring may be a substituted or unsubstituted mono- or polycyclic (3- to 30-membered) alicyclic or aromatic ring, or the combination thereof, which are formed by a linkage of at least two adjacent substituents. In addition, the formed ring may contain at least one heteroatom selected from B, N, O, S, Si, and P, preferably at least one heteroatom selected from N, O, and S. According to one embodiment of the present disclosure, the number of ring backbone atoms is 5 to 20. According to another embodiment of the present disclosure, the number of ring backbone atoms is 5 to 15.

In the formulas of the present disclosure, the heteroaryl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

The present disclosure provides an organic electroluminescent compound represented by formula 1-1 above.

Hereinafter, the compound represented by formula 1-1 will be described in more detail.

In formula 1-1, X₁ to X₃ each independently represent N or CRₐ; with the proviso that at least two of X₁ to X₃ are N. According to one embodiment of the present disclosure, all of X₁ to X₃ may represent N.

In formula 1-1, Rₐ represents hydrogen or deuterium.

In formula 1-1, Ar₁ to Ar₃ each independently represent a (C6-C30)aryl unsubstituted or substituted with at least one of deuterium and a (C6-C30)aryl(s); with the proviso that Ar₁ to Ar₃ are different from each other, and the structure comprising naphthalene and the following structures are excluded.

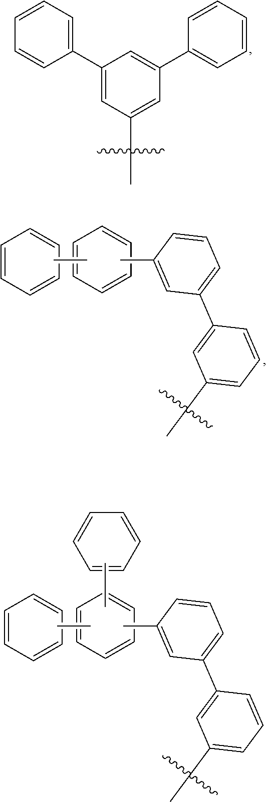

According to one embodiment of the present disclosure, Ar₁ to Ar₃ may each independently represent a (C6-C28)aryl unsubstituted or substituted with at least one of deuterium and a (C6-C30)aryl(s). According to another embodiment of the present disclosure, Ar₁ to Ar₃ may each independently represent a (C6-C25)aryl unsubstituted or substituted with at least one of deuterium and a (C6-C20)aryl(s). For example, Ar₁ to Ar₃ may each independently be a phenyl unsubstituted or substituted with a triphenylenyl(s); a biphenyl; a terphenyl; a quaterphenyl, etc, which may be further substituted with deuterium.

According to still another embodiment of the present disclosure, Ar₁ to Ar₃ may each independently represent a phenyl unsubstituted or substituted with deuterium, a biphenyl unsubstituted or substituted with deuterium, a terphenyl unsubstituted or substituted with deuterium, a quaterphenyl unsubstituted or substituted with deuterium, a phenylnaphthyl unsubstituted or substituted with deuterium, a naphthylphenyl unsubstituted or substituted with deuterium, a triphenylenyl unsubstituted or substituted with deuterium, a phenanthrenyl unsubstituted or substituted with deuterium, or a combination thereof.

According to another embodiment of the present disclosure, Ar₁ to Ar₃ may be all different from each other.

According to one embodiment of the present disclosure, when the compound of formula 1-1 contains deuterium, the deuterium substitution rate thereof may be about 30% to about 100%, preferably about 50% to about 100%, and more preferably about 70% to about 100%. The compound of formula 1-1 having said deuterium substitution rate may increase the bond dissociation energy due to deuteration to increase the stability of the compound, and an organic electroluminescent device comprising the compound may exhibit improved lifespan properties.

The compound represented by formula 1-1 may be selected from the following compounds, but is not limited thereto.

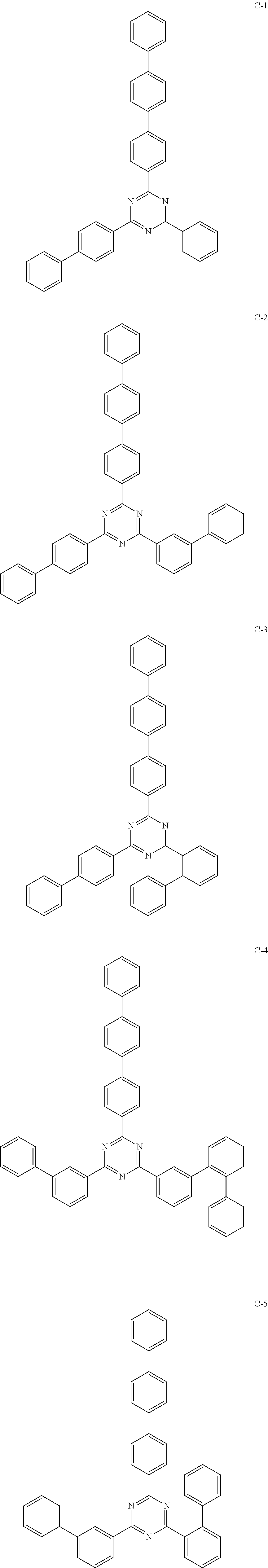 [SMILES: c1ccc(-c2ccc(-c3ccc(-c4nc(-c5ccccc5)nc(-c5ccc(-c6ccccc6)cc5)n4)cc3)cc2)cc1 ; c1ccc(-c2ccc(-c3ccc(-c4nc(-c5ccc(-c6ccccc6)cc5)nc(-c5cccc(-c6ccccc6)c5)n4)cc3)cc2)cc1 ; c1ccc(-c2ccc(-c3ccc(-c4nc(-c5ccc(-c6ccccc6)cc5)nc(-c5ccccc5-c5ccccc5)n4)cc3)cc2)cc1 ; c1ccc(-c2ccc(-c3ccc(-c4nc(-c5cccc(-c6ccccc6)c5)nc(-c5cccc(-c6ccccc6-c6ccccc6)c5)n4)cc3)cc2)cc1 ; c1ccc(-c2ccc(-c3ccc(-c4nc(-c5cccc(-c6ccccc6)c5)nc(-c5ccccc5-c5ccccc5)n4)cc3)cc2)cc1]

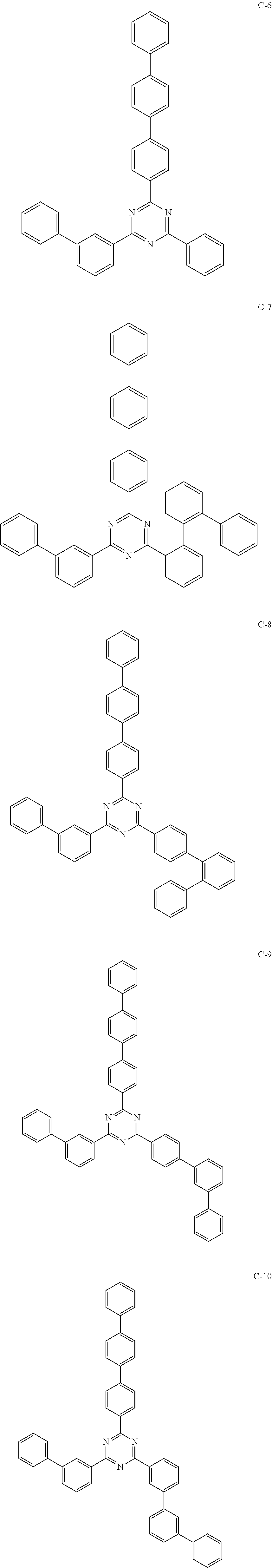 [SMILES: c1ccc(-c2ccc(-c3ccc(-c4nc(-c5ccccc5)nc(-c5cccc(-c6ccccc6)c5)n4)cc3)cc2)cc1 ; c1ccc(-c2ccc(-c3ccc(-c4nc(-c5cccc(-c6ccccc6)c5)nc(-c5ccccc5-c5ccccc5-c5ccccc5)n4)cc3)cc2)cc1 ; c1ccc(-c2ccc(-c3ccc(-c4nc(-c5ccc(-c6ccccc6-c6ccccc6)cc5)nc(-c5cccc(-c6ccccc6)c5)n4)cc3)cc2)cc1 ; c1ccc(-c2ccc(-c3ccc(-c4nc(-c5ccc(-c6cccc(-c7ccccc7)c6)cc5)nc(-c5cccc(-c6ccccc6)c5)n4)cc3)cc2)cc1 ; c1ccc(-c2ccc(-c3ccc(-c4nc(-c5cccc(-c6ccccc6)c5)nc(-c5cccc(-c6cccc(-c7ccccc7)c6)c5)n4)cc3)cc2)cc1]

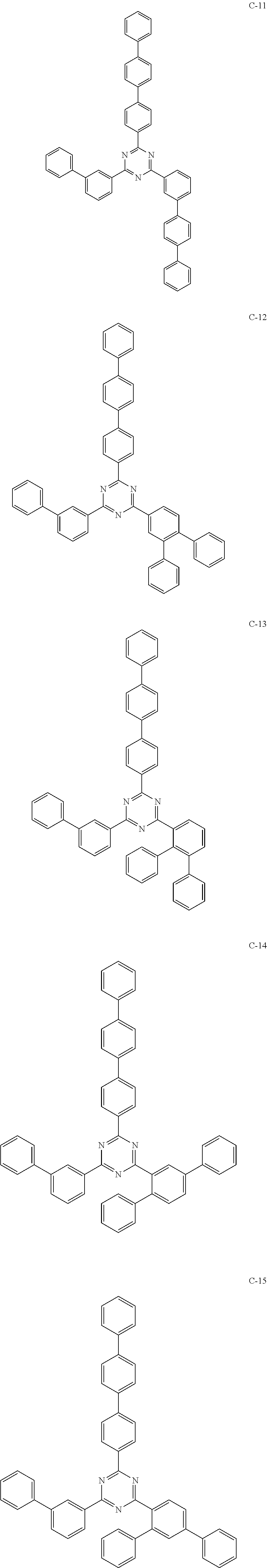

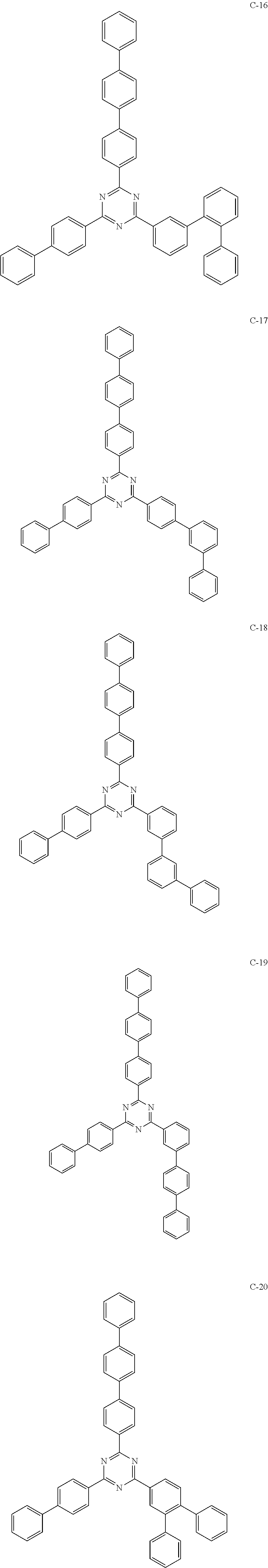 [SMILES: c1ccc(-c2ccc(-c3ccc(-c4nc(-c5ccc(-c6ccccc6)cc5)nc(-c5cccc(-c6ccccc6-c6ccccc6)c5)n4)cc3)cc2)cc1 ; c1ccc(-c2ccc(-c3ccc(-c4nc(-c5ccc(-c6ccccc6)cc5)nc(-c5ccc(-c6cccc(-c7ccccc7)c6)cc5)n4)cc3)cc2)cc1 ; c1ccc(-c2ccc(-c3ccc(-c4nc(-c5ccc(-c6ccccc6)cc5)nc(-c5cccc(-c6cccc(-c7ccccc7)c6)c5)n4)cc3)cc2)cc1 ; c1ccc(-c2ccc(-c3ccc(-c4nc(-c5ccc(-c6ccccc6)cc5)nc(-c5cccc(-c6ccc(-c7ccccc7)cc6)c5)n4)cc3)cc2)cc1 ; c1ccc(-c2ccc(-c3ccc(-c4nc(-c5ccc(-c6ccccc6)cc5)nc(-c5ccc(-c6ccccc6)c(-c6ccccc6)c5)n4)cc3)cc2)cc1]

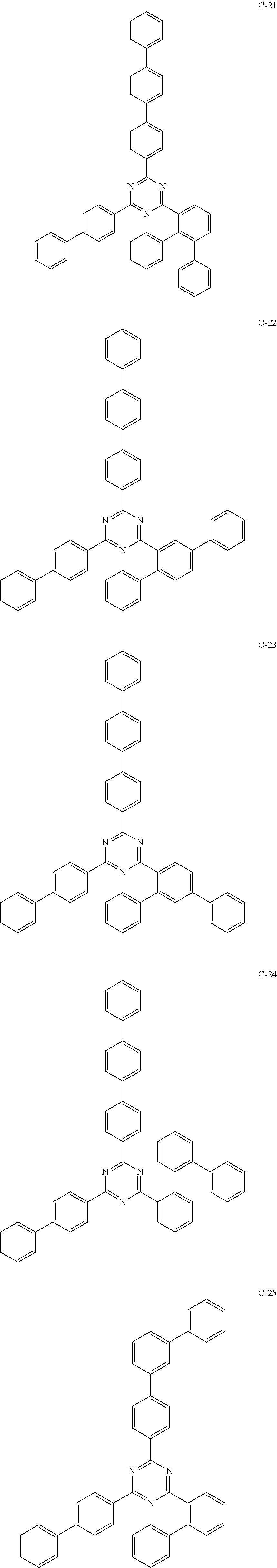

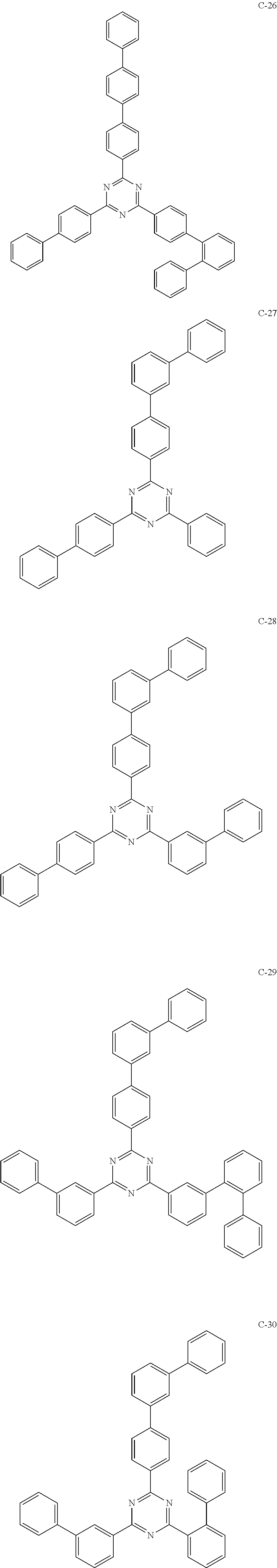 [SMILES: c1ccc(-c2ccc(-c3ccc(-c4nc(-c5ccc(-c6ccccc6)cc5)nc(-c5ccc(-c6ccccc6-c6ccccc6)cc5)n4)cc3)cc2)cc1 ; c1ccc(-c2ccc(-c3nc(-c4ccccc4)nc(-c4ccc(-c5cccc(-c6ccccc6)c5)cc4)n3)cc2)cc1 ; c1ccc(-c2ccc(-c3nc(-c4ccc(-c5cccc(-c6ccccc6)c5)cc4)nc(-c4cccc(-c5ccccc5)c4)n3)cc2)cc1 ; c1ccc(-c2cccc(-c3ccc(-c4nc(-c5cccc(-c6ccccc6)c5)nc(-c5cccc(-c6ccccc6-c6ccccc6)c5)n4)cc3)c2)cc1 ; c1ccc(-c2cccc(-c3ccc(-c4nc(-c5cccc(-c6ccccc6)c5)nc(-c5ccccc5-c5ccccc5)n4)cc3)c2)cc1]

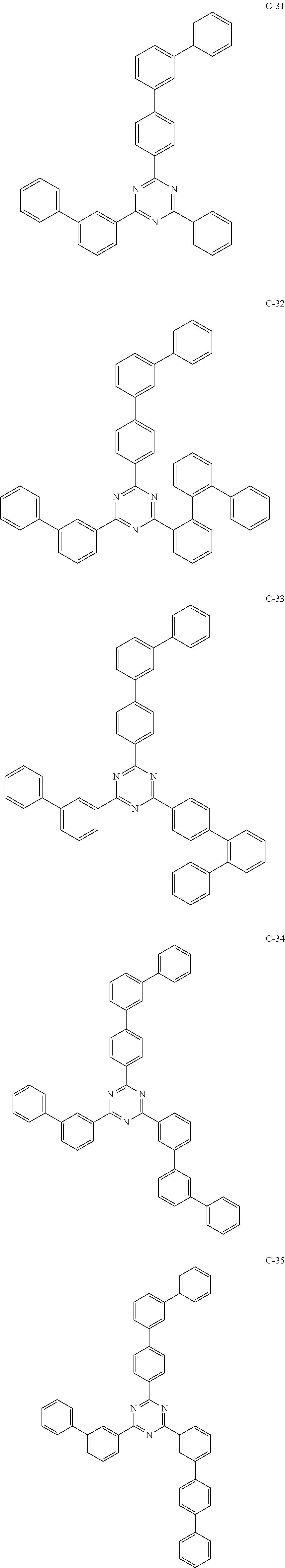 [SMILES: c1ccc(-c2cccc(-c3ccc(-c4nc(-c5ccccc5)nc(-c5cccc(-c6ccccc6)c5)n4)cc3)c2)cc1 ; c1ccc(-c2cccc(-c3ccc(-c4nc(-c5cccc(-c6ccccc6)c5)nc(-c5ccccc5-c5ccccc5-c5ccccc5)n4)cc3)c2)cc1 ; c1ccc(-c2cccc(-c3ccc(-c4nc(-c5ccc(-c6ccccc6-c6ccccc6)cc5)nc(-c5cccc(-c6ccccc6)c5)n4)cc3)c2)cc1 ; c1ccc(-c2cccc(-c3ccc(-c4nc(-c5cccc(-c6ccccc6)c5)nc(-c5cccc(-c6cccc(-c7ccccc7)c6)c5)n4)cc3)c2)cc1 ; c1ccc(-c2ccc(-c3cccc(-c4nc(-c5ccc(-c6cccc(-c7ccccc7)c6)cc5)nc(-c5cccc(-c6ccccc6)c5)n4)c3)cc2)cc1]

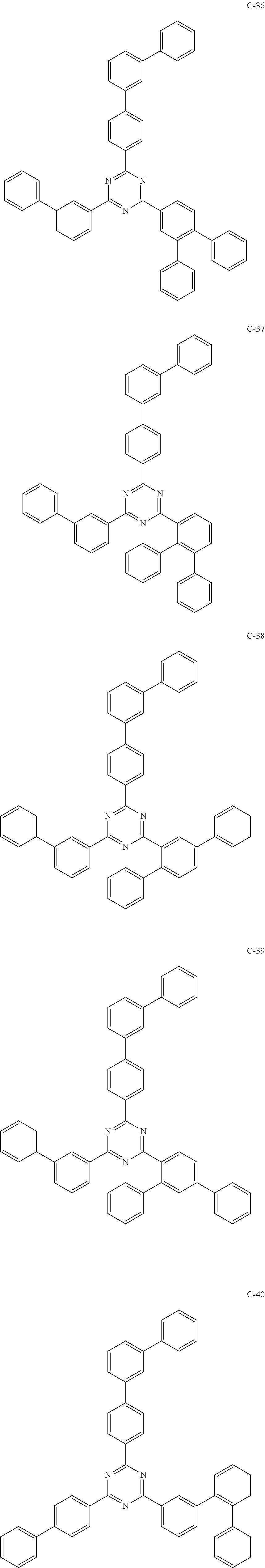 [SMILES: c1ccc(-c2cccc(-c3ccc(-c4nc(-c5cccc(-c6ccccc6)c5)nc(-c5ccc(-c6ccccc6)c(-c6ccccc6)c5)n4)cc3)c2)cc1 ; c1ccc(-c2cccc(-c3ccc(-c4nc(-c5cccc(-c6ccccc6)c5)nc(-c5cccc(-c6ccccc6)c5-c5ccccc5)n4)cc3)c2)cc1 ; c1ccc(-c2cccc(-c3ccc(-c4nc(-c5cccc(-c6ccccc6)c5)nc(-c5cc(-c6ccccc6)ccc5-c5ccccc5)n4)cc3)c2)cc1 ; c1ccc(-c2cccc(-c3ccc(-c4nc(-c5cccc(-c6ccccc6)c5)nc(-c5ccc(-c6ccccc6)cc5-c5ccccc5)n4)cc3)c2)cc1 ; c1ccc(-c2ccc(-c3nc(-c4ccc(-c5cccc(-c6ccccc6)c5)cc4)nc(-c4cccc(-c5ccccc5-c5ccccc5)c4)n3)cc2)cc1]

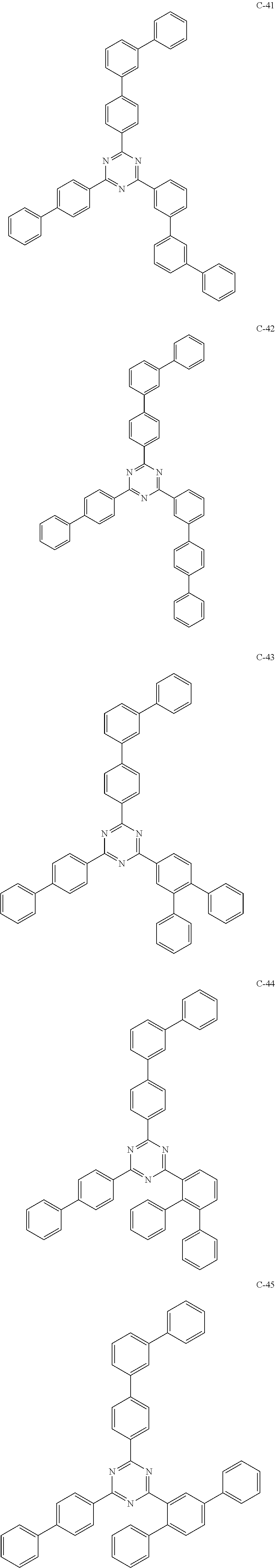

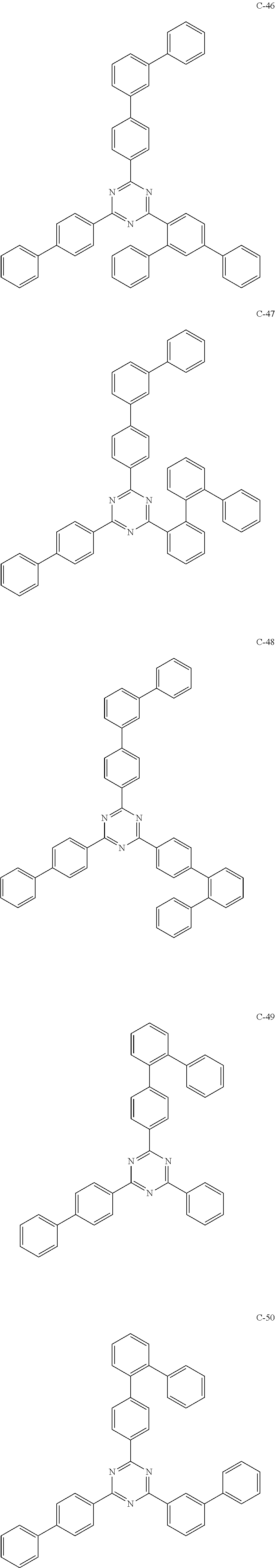 [SMILES: c1ccc(-c2ccc(-c3nc(-c4ccc(-c5cccc(-c6ccccc6)c5)cc4)nc(-c4ccc(-c5ccccc5)cc4-c4ccccc4)n3)cc2)cc1 ; c1ccc(-c2ccc(-c3nc(-c4ccc(-c5cccc(-c6ccccc6)c5)cc4)nc(-c4ccccc4-c4ccccc4-c4ccccc4)n3)cc2)cc1 ; c1ccc(-c2ccc(-c3nc(-c4ccc(-c5cccc(-c6ccccc6)c5)cc4)nc(-c4ccc(-c5ccccc5-c5ccccc5)cc4)n3)cc2)cc1 ; c1ccc(-c2ccc(-c3nc(-c4ccccc4)nc(-c4ccc(-c5ccccc5-c5ccccc5)cc4)n3)cc2)cc1 ; c1ccc(-c2ccc(-c3nc(-c4ccc(-c5ccccc5-c5ccccc5)cc4)nc(-c4cccc(-c5ccccc5)c4)n3)cc2)cc1]

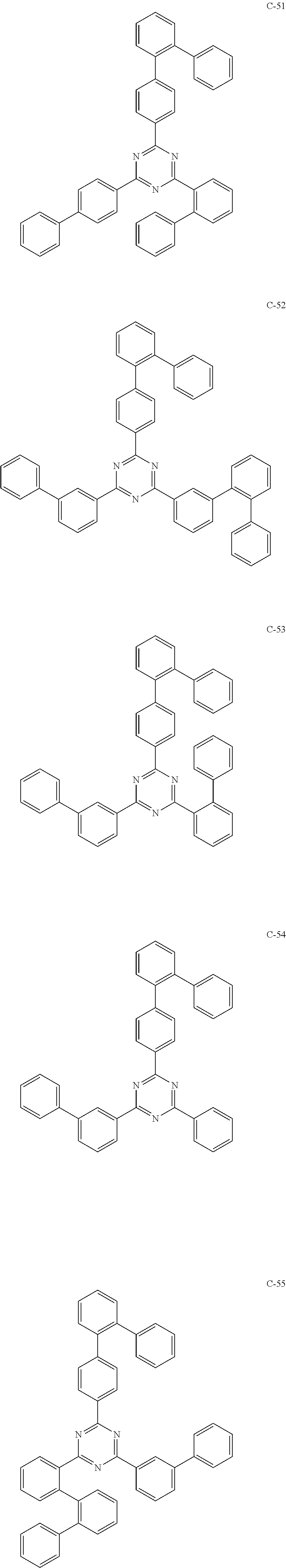 [SMILES: c1ccc(-c2ccc(-c3nc(-c4ccc(-c5ccccc5-c5ccccc5)cc4)nc(-c4ccccc4-c4ccccc4)n3)cc2)cc1 ; c1ccc(-c2cccc(-c3nc(-c4ccc(-c5ccccc5-c5ccccc5)cc4)nc(-c4cccc(-c5ccccc5-c5ccccc5)c4)n3)c2)cc1 ; c1ccc(-c2cccc(-c3nc(-c4ccc(-c5ccccc5-c5ccccc5)cc4)nc(-c4ccccc4-c4ccccc4)n3)c2)cc1 ; c1ccc(-c2cccc(-c3nc(-c4ccccc4)nc(-c4ccc(-c5ccccc5-c5ccccc5)cc4)n3)c2)cc1 ; c1ccc(-c2cccc(-c3nc(-c4ccc(-c5ccccc5-c5ccccc5)cc4)nc(-c4ccccc4-c4ccccc4-c4ccccc4)n3)c2)cc1]

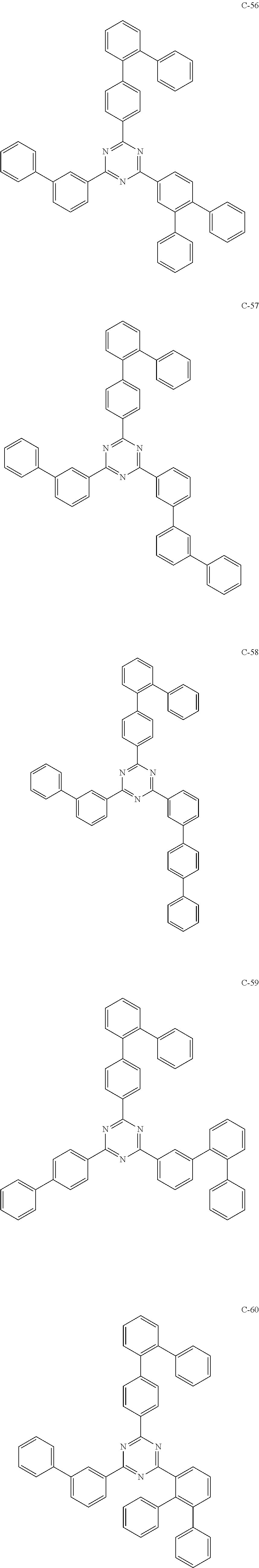

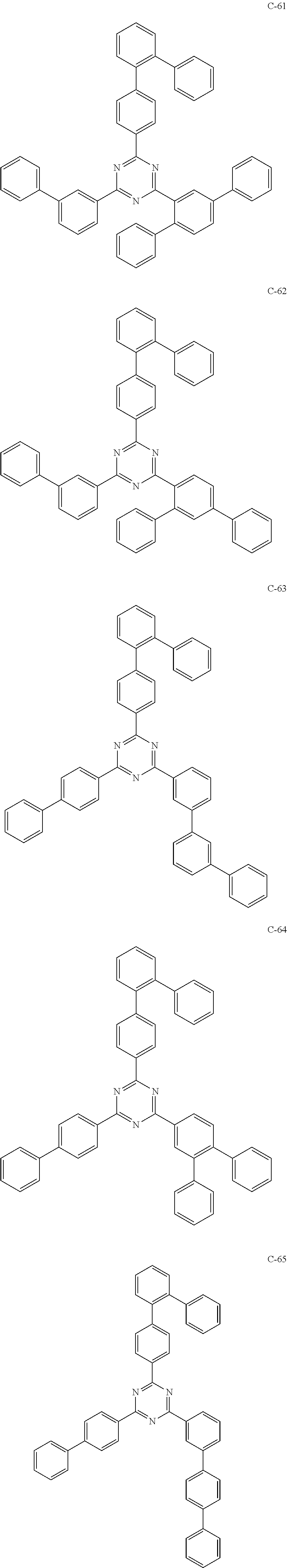 [SMILES: c1ccc(-c2cccc(-c3nc(-c4ccc(-c5ccccc5-c5ccccc5)cc4)nc(-c4cc(-c5ccccc5)ccc4-c4ccccc4)n3)c2)cc1 ; c1ccc(-c2cccc(-c3nc(-c4ccc(-c5ccccc5-c5ccccc5)cc4)nc(-c4ccc(-c5ccccc5)cc4-c4ccccc4)n3)c2)cc1 ; c1ccc(-c2ccc(-c3nc(-c4ccc(-c5ccccc5-c5ccccc5)cc4)nc(-c4cccc(-c5cccc(-c6ccccc6)c5)c4)n3)cc2)cc1 ; c1ccc(-c2ccc(-c3nc(-c4ccc(-c5ccccc5-c5ccccc5)cc4)nc(-c4ccc(-c5ccccc5)c(-c5ccccc5)c4)n3)cc2)cc1 ; c1ccc(-c2ccc(-c3cccc(-c4nc(-c5ccc(-c6ccccc6)cc5)nc(-c5ccc(-c6ccccc6-c6ccccc6)cc5)n4)c3)cc2)cc1]

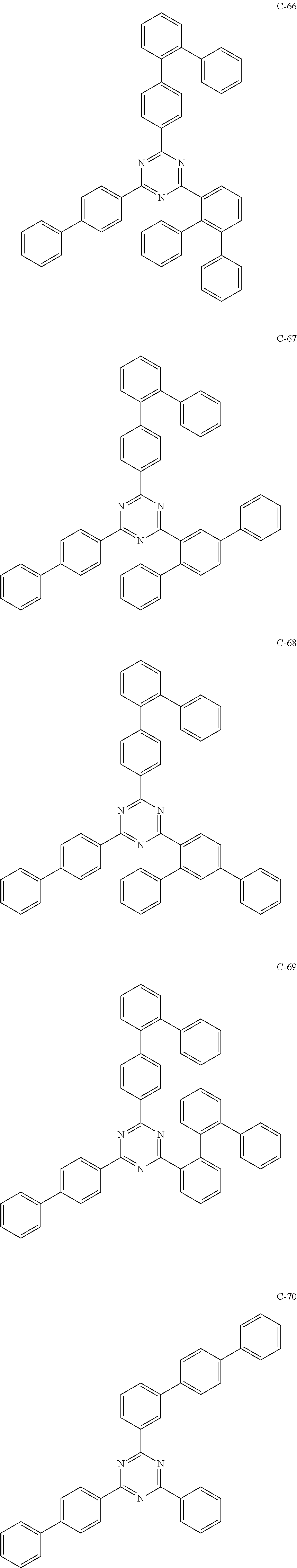 [SMILES: c1ccc(-c2ccc(-c3nc(-c4ccc(-c5ccccc5-c5ccccc5)cc4)nc(-c4cccc(-c5ccccc5)c4-c4ccccc4)n3)cc2)cc1 ; c1ccc(-c2ccc(-c3nc(-c4ccc(-c5ccccc5-c5ccccc5)cc4)nc(-c4cc(-c5ccccc5)ccc4-c4ccccc4)n3)cc2)cc1 ; c1ccc(-c2ccc(-c3nc(-c4ccc(-c5ccccc5-c5ccccc5)cc4)nc(-c4ccc(-c5ccccc5)cc4-c4ccccc4)n3)cc2)cc1 ; c1ccc(-c2ccc(-c3nc(-c4ccc(-c5ccccc5-c5ccccc5)cc4)nc(-c4ccccc4-c4ccccc4-c4ccccc4)n3)cc2)cc1 ; c1ccc(-c2ccc(-c3cccc(-c4nc(-c5ccccc5)nc(-c5ccc(-c6ccccc6)cc5)n4)c3)cc2)cc1]

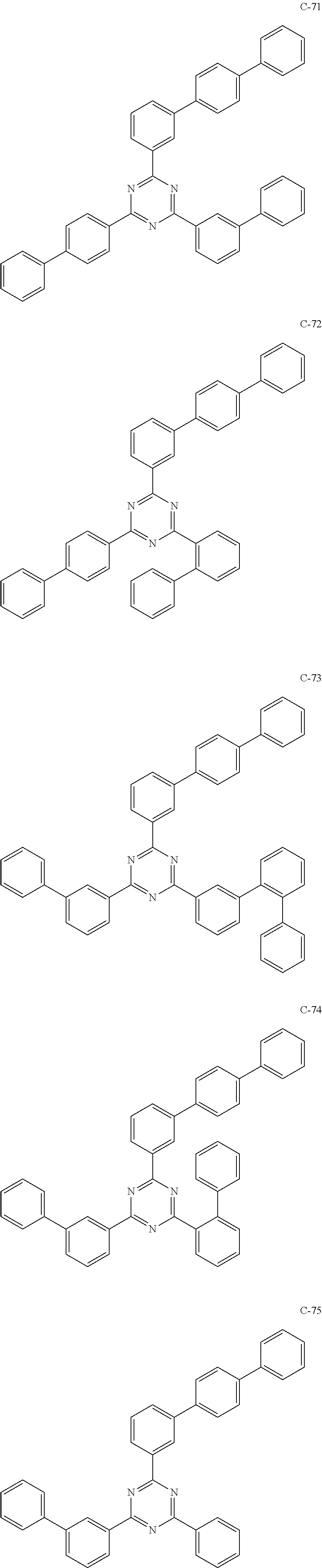 [SMILES: c1ccc(-c2ccc(-c3cccc(-c4nc(-c5ccc(-c6ccccc6)cc5)nc(-c5cccc(-c6ccccc6)c5)n4)c3)cc2)cc1 ; c1ccc(-c2ccc(-c3cccc(-c4nc(-c5ccc(-c6ccccc6)cc5)nc(-c5ccccc5-c5ccccc5)n4)c3)cc2)cc1 ; c1ccc(-c2ccc(-c3cccc(-c4nc(-c5cccc(-c6ccccc6)c5)nc(-c5cccc(-c6ccccc6-c6ccccc6)c5)n4)c3)cc2)cc1 ; c1ccc(-c2ccc(-c3cccc(-c4nc(-c5cccc(-c6ccccc6)c5)nc(-c5ccccc5-c5ccccc5)n4)c3)cc2)cc1 ; c1ccc(-c2ccc(-c3cccc(-c4nc(-c5ccccc5)nc(-c5cccc(-c6ccccc6)c5)n4)c3)cc2)cc1]

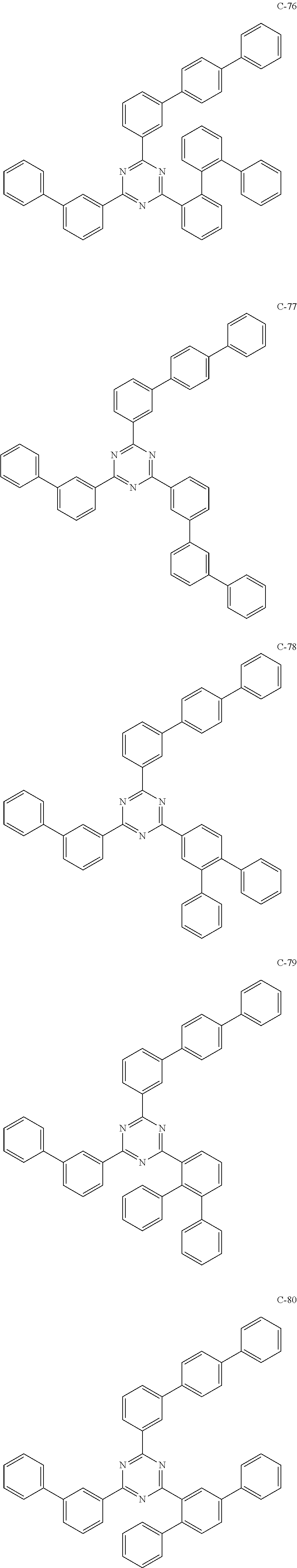 [SMILES: c1ccc(-c2ccc(-c3cccc(-c4nc(-c5cccc(-c6ccccc6)c5)nc(-c5ccccc5-c5ccccc5-c5ccccc5)n4)c3)cc2)cc1 ; c1ccc(-c2ccc(-c3cccc(-c4nc(-c5cccc(-c6ccccc6)c5)nc(-c5cccc(-c6cccc(-c7ccccc7)c6)c5)n4)c3)cc2)cc1 ; c1ccc(-c2ccc(-c3cccc(-c4nc(-c5cccc(-c6ccccc6)c5)nc(-c5ccc(-c6ccccc6)c(-c6ccccc6)c5)n4)c3)cc2)cc1 ; c1ccc(-c2ccc(-c3cccc(-c4nc(-c5cccc(-c6ccccc6)c5)nc(-c5cccc(-c6ccccc6)c5-c5ccccc5)n4)c3)cc2)cc1 ; c1ccc(-c2ccc(-c3cccc(-c4nc(-c5cccc(-c6ccccc6)c5)nc(-c5cc(-c6ccccc6)ccc5-c5ccccc5)n4)c3)cc2)cc1]

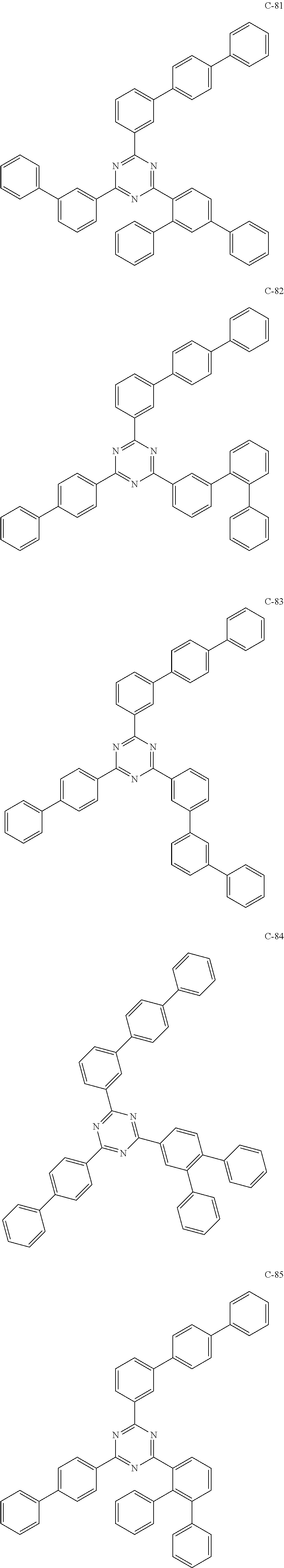 [SMILES: c1ccc(-c2ccc(-c3cccc(-c4nc(-c5cccc(-c6ccccc6)c5)nc(-c5ccc(-c6ccccc6)cc5-c5ccccc5)n4)c3)cc2)cc1 ; c1ccc(-c2ccc(-c3cccc(-c4nc(-c5ccc(-c6ccccc6)cc5)nc(-c5cccc(-c6ccccc6-c6ccccc6)c5)n4)c3)cc2)cc1 ; c1ccc(-c2ccc(-c3cccc(-c4nc(-c5ccc(-c6ccccc6)cc5)nc(-c5cccc(-c6cccc(-c7ccccc7)c6)c5)n4)c3)cc2)cc1 ; c1ccc(-c2ccc(-c3cccc(-c4nc(-c5ccc(-c6ccccc6)cc5)nc(-c5ccc(-c6ccccc6)c(-c6ccccc6)c5)n4)c3)cc2)cc1 ; c1ccc(-c2ccc(-c3cccc(-c4nc(-c5ccc(-c6ccccc6)cc5)nc(-c5cccc(-c6ccccc6)c5-c5ccccc5)n4)c3)cc2)cc1]

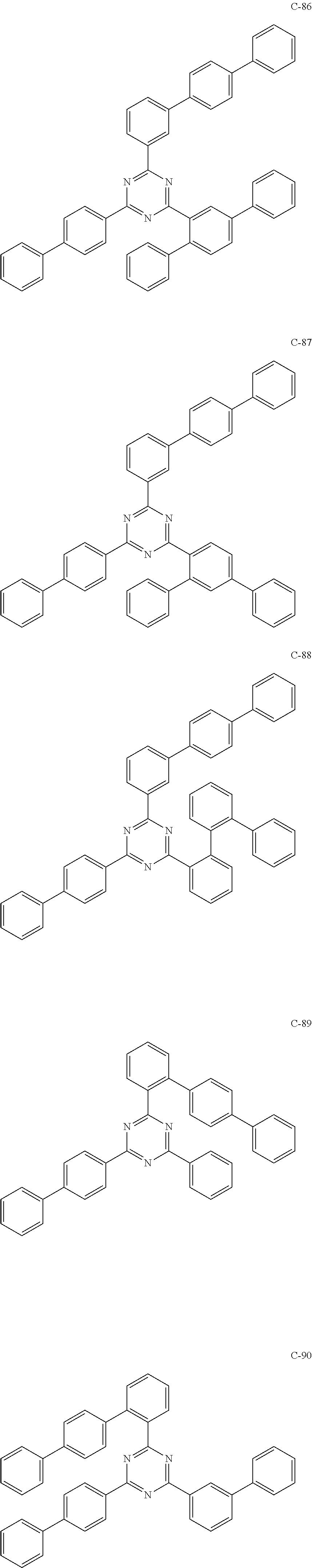 [SMILES: c1ccc(-c2ccc(-c3cccc(-c4nc(-c5ccc(-c6ccccc6)cc5)nc(-c5cc(-c6ccccc6)ccc5-c5ccccc5)n4)c3)cc2)cc1 ; c1ccc(-c2ccc(-c3cccc(-c4nc(-c5ccc(-c6ccccc6)cc5)nc(-c5ccc(-c6ccccc6)cc5-c5ccccc5)n4)c3)cc2)cc1 ; c1ccc(-c2ccc(-c3cccc(-c4nc(-c5ccc(-c6ccccc6)cc5)nc(-c5ccccc5-c5ccccc5-c5ccccc5)n4)c3)cc2)cc1 ; c1ccc(-c2ccc(-c3nc(-c4ccccc4)nc(-c4ccccc4-c4ccc(-c5ccccc5)cc4)n3)cc2)cc1 ; c1ccc(-c2ccc(-c3nc(-c4cccc(-c5ccccc5)c4)nc(-c4ccccc4-c4ccc(-c5ccccc5)cc4)n3)cc2)cc1]

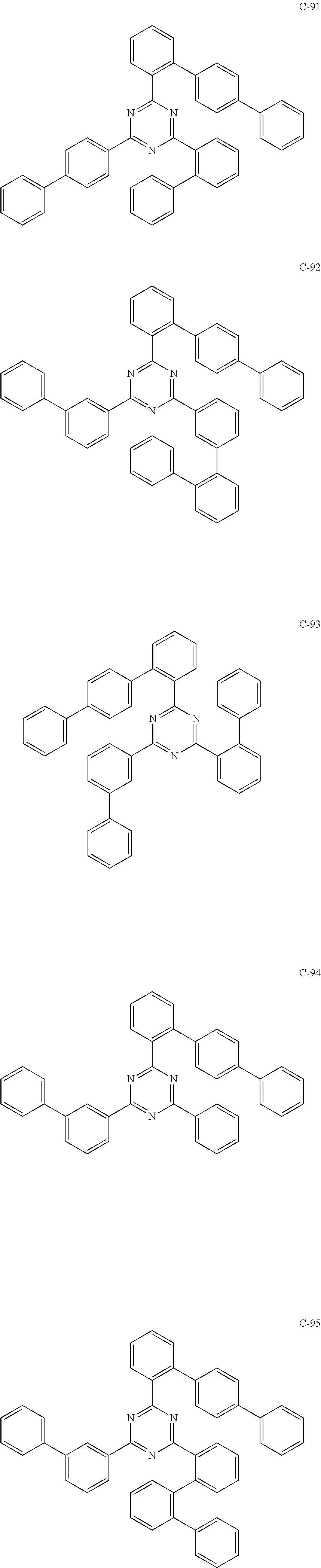 [SMILES: c1ccc(-c2ccc(-c3nc(-c4ccccc4-c4ccccc4)nc(-c4ccccc4-c4ccc(-c5ccccc5)cc4)n3)cc2)cc1 ; c1ccc(-c2ccc(-c3ccccc3-c3nc(-c4cccc(-c5ccccc5)c4)nc(-c4cccc(-c5ccccc5-c5ccccc5)c4)n3)cc2)cc1 ; c1ccc(-c2ccc(-c3ccccc3-c3nc(-c4cccc(-c5ccccc5)c4)nc(-c4ccccc4-c4ccccc4)n3)cc2)cc1 ; c1ccc(-c2ccc(-c3ccccc3-c3nc(-c4ccccc4)nc(-c4cccc(-c5ccccc5)c4)n3)cc2)cc1 ; c1ccc(-c2ccc(-c3ccccc3-c3nc(-c4cccc(-c5ccccc5)c4)nc(-c4ccccc4-c4ccccc4-c4ccccc4)n3)cc2)cc1]

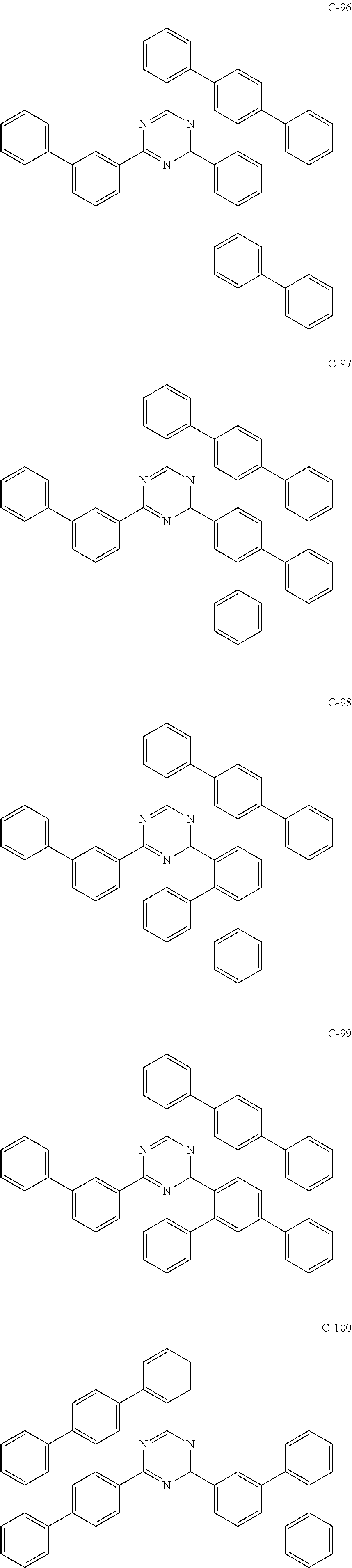 [SMILES: c1ccc(-c2ccc(-c3ccccc3-c3nc(-c4cccc(-c5ccccc5)c4)nc(-c4cccc(-c5cccc(-c6ccccc6)c5)c4)n3)cc2)cc1 ; c1ccc(-c2ccc(-c3ccccc3-c3nc(-c4cccc(-c5ccccc5)c4)nc(-c4ccc(-c5ccccc5)c(-c5ccccc5)c4)n3)cc2)cc1 ; c1ccc(-c2ccc(-c3ccccc3-c3nc(-c4cccc(-c5ccccc5)c4)nc(-c4cccc(-c5ccccc5)c4-c4ccccc4)n3)cc2)cc1 ; c1ccc(-c2ccc(-c3ccccc3-c3nc(-c4cccc(-c5ccccc5)c4)nc(-c4ccc(-c5ccccc5)cc4-c4ccccc4)n3)cc2)cc1 ; c1ccc(-c2ccc(-c3nc(-c4cccc(-c5ccccc5-c5ccccc5)c4)nc(-c4ccccc4-c4ccc(-c5ccccc5)cc4)n3)cc2)cc1]

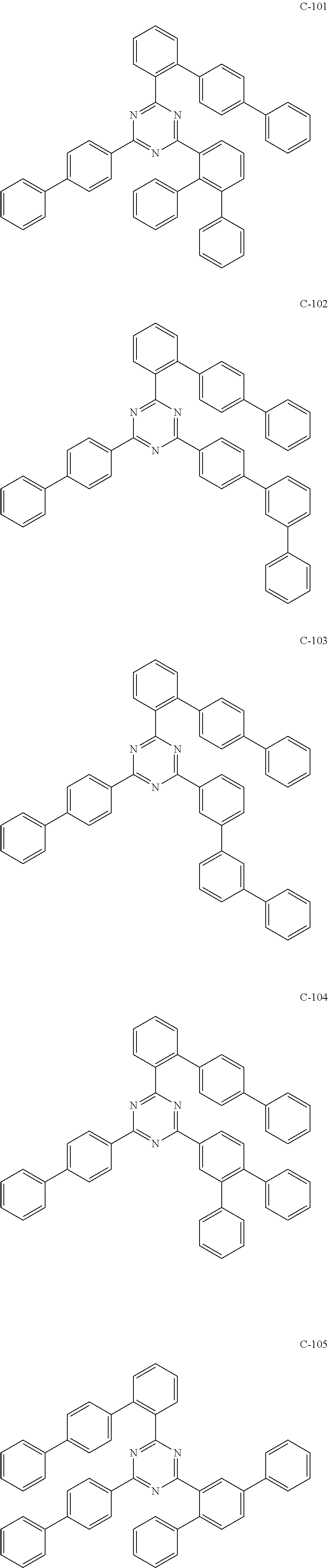 [SMILES: c1ccc(-c2ccc(-c3nc(-c4ccccc4-c4ccc(-c5ccccc5)cc4)nc(-c4cccc(-c5ccccc5)c4-c4ccccc4)n3)cc2)cc1 ; c1ccc(-c2ccc(-c3nc(-c4ccc(-c5cccc(-c6ccccc6)c5)cc4)nc(-c4ccccc4-c4ccc(-c5ccccc5)cc4)n3)cc2)cc1 ; c1ccc(-c2ccc(-c3nc(-c4cccc(-c5cccc(-c6ccccc6)c5)c4)nc(-c4ccccc4-c4ccc(-c5ccccc5)cc4)n3)cc2)cc1 ; c1ccc(-c2ccc(-c3nc(-c4ccc(-c5ccccc5)c(-c5ccccc5)c4)nc(-c4ccccc4-c4ccc(-c5ccccc5)cc4)n3)cc2)cc1 ; c1ccc(-c2ccc(-c3nc(-c4ccccc4-c4ccc(-c5ccccc5)cc4)nc(-c4cc(-c5ccccc5)ccc4-c4ccccc4)n3)cc2)cc1]

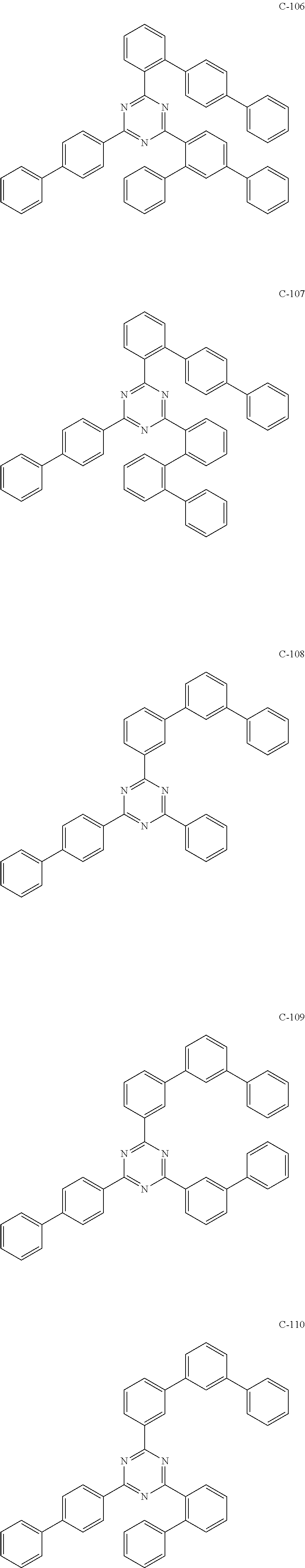 [SMILES: c1ccc(-c2ccc(-c3nc(-c4ccccc4-c4ccc(-c5ccccc5)cc4)nc(-c4ccc(-c5ccccc5)cc4-c4ccccc4)n3)cc2)cc1 ; c1ccc(-c2ccc(-c3nc(-c4ccccc4-c4ccc(-c5ccccc5)cc4)nc(-c4ccccc4-c4ccccc4-c4ccccc4)n3)cc2)cc1 ; c1ccc(-c2ccc(-c3nc(-c4ccccc4)nc(-c4cccc(-c5cccc(-c6ccccc6)c5)c4)n3)cc2)cc1 ; c1ccc(-c2ccc(-c3nc(-c4cccc(-c5ccccc5)c4)nc(-c4cccc(-c5cccc(-c6ccccc6)c5)c4)n3)cc2)cc1 ; c1ccc(-c2ccc(-c3nc(-c4cccc(-c5cccc(-c6ccccc6)c5)c4)nc(-c4ccccc4-c4ccccc4)n3)cc2)cc1]

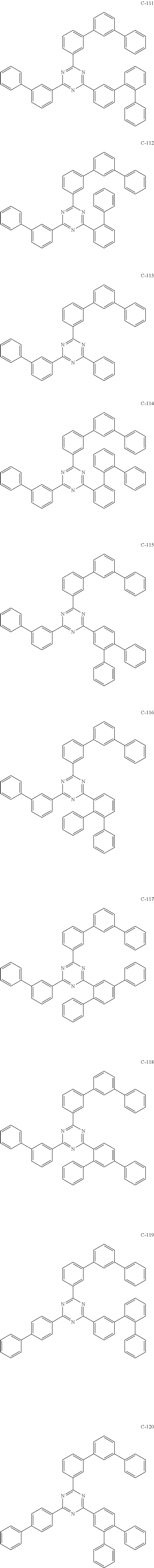

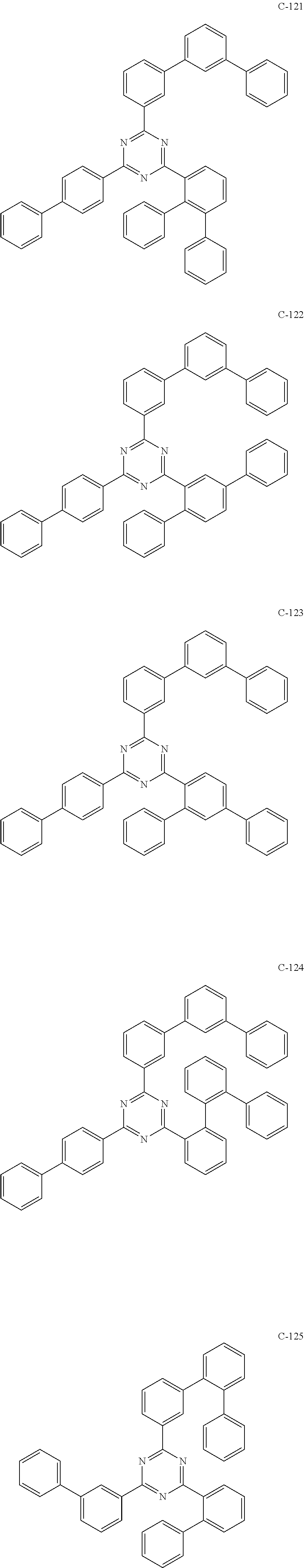 [SMILES: c1ccc(-c2ccc(-c3nc(-c4cccc(-c5cccc(-c6ccccc6)c5)c4)nc(-c4cccc(-c5ccccc5)c4-c4ccccc4)n3)cc2)cc1 ; c1ccc(-c2ccc(-c3nc(-c4cccc(-c5cccc(-c6ccccc6)c5)c4)nc(-c4cc(-c5ccccc5)ccc4-c4ccccc4)n3)cc2)cc1 ; c1ccc(-c2ccc(-c3nc(-c4cccc(-c5cccc(-c6ccccc6)c5)c4)nc(-c4ccc(-c5ccccc5)cc4-c4ccccc4)n3)cc2)cc1 ; c1ccc(-c2ccc(-c3nc(-c4cccc(-c5cccc(-c6ccccc6)c5)c4)nc(-c4ccccc4-c4ccccc4-c4ccccc4)n3)cc2)cc1 ; c1ccc(-c2cccc(-c3nc(-c4cccc(-c5ccccc5-c5ccccc5)c4)nc(-c4ccccc4-c4ccccc4)n3)c2)cc1]

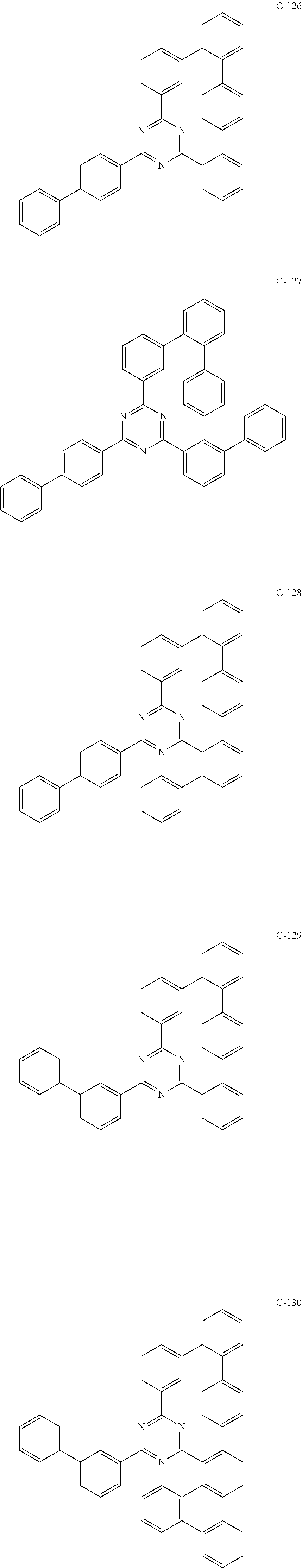 [SMILES: c1ccc(-c2ccc(-c3nc(-c4ccccc4)nc(-c4cccc(-c5ccccc5-c5ccccc5)c4)n3)cc2)cc1 ; c1ccc(-c2ccc(-c3nc(-c4cccc(-c5ccccc5)c4)nc(-c4cccc(-c5ccccc5-c5ccccc5)c4)n3)cc2)cc1 ; c1ccc(-c2ccc(-c3nc(-c4cccc(-c5ccccc5-c5ccccc5)c4)nc(-c4ccccc4-c4ccccc4)n3)cc2)cc1 ; c1ccc(-c2cccc(-c3nc(-c4ccccc4)nc(-c4cccc(-c5ccccc5-c5ccccc5)c4)n3)c2)cc1 ; c1ccc(-c2cccc(-c3nc(-c4cccc(-c5ccccc5-c5ccccc5)c4)nc(-c4ccccc4-c4ccccc4-c4ccccc4)n3)c2)cc1]

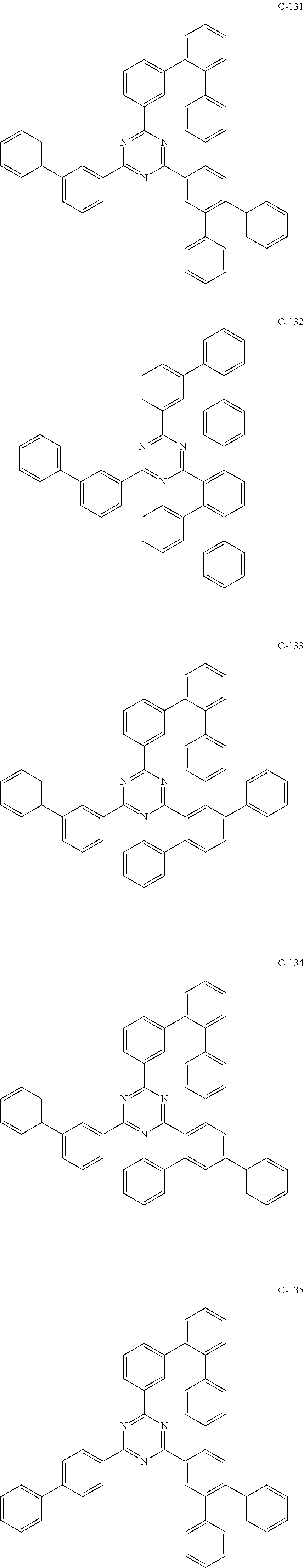 [SMILES: c1ccc(-c2cccc(-c3nc(-c4cccc(-c5ccccc5-c5ccccc5)c4)nc(-c4ccc(-c5ccccc5)c(-c5ccccc5)c4)n3)c2)cc1 ; c1ccc(-c2cccc(-c3nc(-c4cccc(-c5ccccc5-c5ccccc5)c4)nc(-c4cccc(-c5ccccc5)c4-c4ccccc4)n3)c2)cc1 ; c1ccc(-c2cccc(-c3nc(-c4cccc(-c5ccccc5-c5ccccc5)c4)nc(-c4cc(-c5ccccc5)ccc4-c4ccccc4)n3)c2)cc1 ; c1ccc(-c2cccc(-c3nc(-c4cccc(-c5ccccc5-c5ccccc5)c4)nc(-c4ccc(-c5ccccc5)cc4-c4ccccc4)n3)c2)cc1 ; c1ccc(-c2ccc(-c3nc(-c4cccc(-c5ccccc5-c5ccccc5)c4)nc(-c4ccc(-c5ccccc5)c(-c5ccccc5)c4)n3)cc2)cc1]

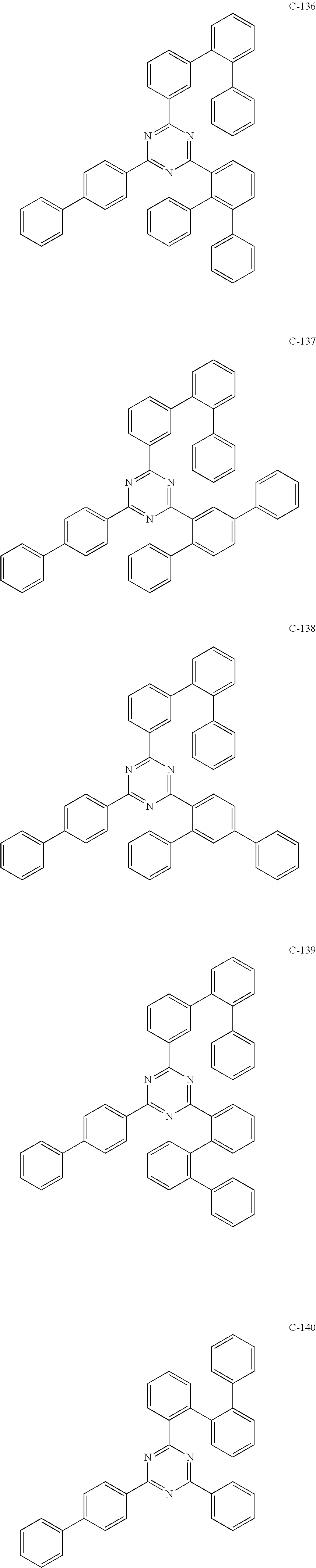 [SMILES: c1ccc(-c2ccc(-c3nc(-c4cccc(-c5ccccc5-c5ccccc5)c4)nc(-c4cccc(-c5ccccc5)c4-c4ccccc4)n3)cc2)cc1 ; c1ccc(-c2ccc(-c3nc(-c4cccc(-c5ccccc5-c5ccccc5)c4)nc(-c4cc(-c5ccccc5)ccc4-c4ccccc4)n3)cc2)cc1 ; c1ccc(-c2ccc(-c3nc(-c4cccc(-c5ccccc5-c5ccccc5)c4)nc(-c4ccc(-c5ccccc5)cc4-c4ccccc4)n3)cc2)cc1 ; c1ccc(-c2ccc(-c3nc(-c4cccc(-c5ccccc5-c5ccccc5)c4)nc(-c4ccccc4-c4ccccc4-c4ccccc4)n3)cc2)cc1 ; c1ccc(-c2ccc(-c3nc(-c4ccccc4)nc(-c4ccccc4-c4ccccc4-c4ccccc4)n3)cc2)cc1]

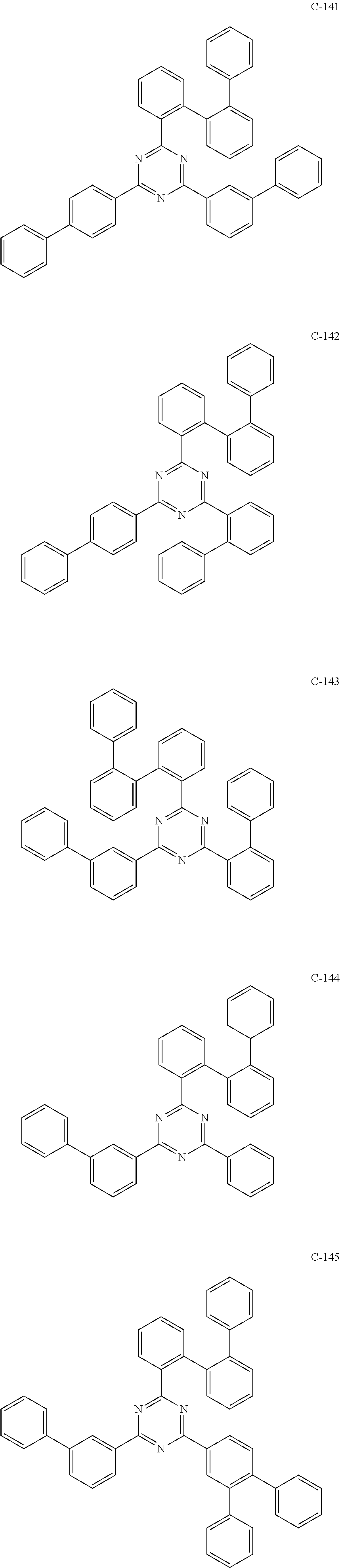 [SMILES: c1ccc(-c2ccc(-c3nc(-c4cccc(-c5ccccc5)c4)nc(-c4ccccc4-c4ccccc4-c4ccccc4)n3)cc2)cc1 ; c1ccc(-c2ccc(-c3nc(-c4ccccc4-c4ccccc4)nc(-c4ccccc4-c4ccccc4-c4ccccc4)n3)cc2)cc1 ; c1ccc(-c2cccc(-c3nc(-c4ccccc4-c4ccccc4)nc(-c4ccccc4-c4ccccc4-c4ccccc4)n3)c2)cc1 ; C1=CCC(c2ccccc2-c2ccccc2-c2nc(-c3ccccc3)nc(-c3cccc(-c4ccccc4)c3)n2)C=C1 ; c1ccc(-c2cccc(-c3nc(-c4ccc(-c5ccccc5)c(-c5ccccc5)c4)nc(-c4ccccc4-c4ccccc4-c4ccccc4)n3)c2)cc1]

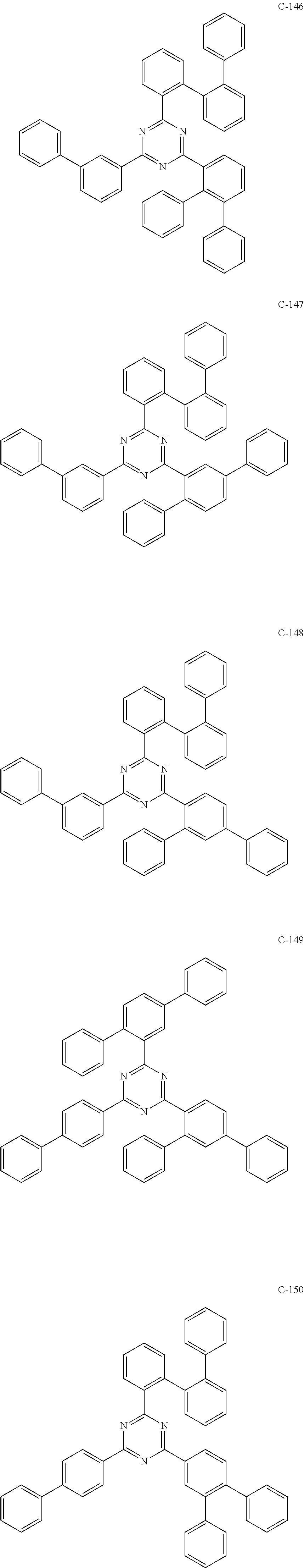 [SMILES: c1ccc(-c2cccc(-c3nc(-c4ccccc4-c4ccccc4-c4ccccc4)nc(-c4cccc(-c5ccccc5)c4-c4ccccc4)n3)c2)cc1 ; c1ccc(-c2cccc(-c3nc(-c4cc(-c5ccccc5)ccc4-c4ccccc4)nc(-c4ccccc4-c4ccccc4-c4ccccc4)n3)c2)cc1 ; c1ccc(-c2cccc(-c3nc(-c4ccc(-c5ccccc5)cc4-c4ccccc4)nc(-c4ccccc4-c4ccccc4-c4ccccc4)n3)c2)cc1 ; c1ccc(-c2ccc(-c3nc(-c4ccc(-c5ccccc5)cc4-c4ccccc4)nc(-c4cc(-c5ccccc5)ccc4-c4ccccc4)n3)cc2)cc1 ; c1ccc(-c2ccc(-c3nc(-c4ccc(-c5ccccc5)c(-c5ccccc5)c4)nc(-c4ccccc4-c4ccccc4-c4ccccc4)n3)cc2)cc1]

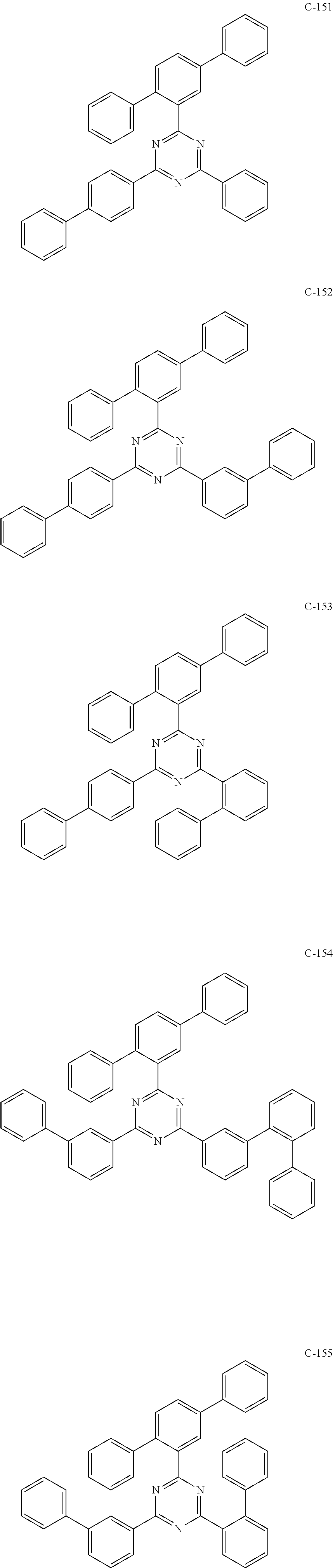 [SMILES: c1ccc(-c2ccc(-c3nc(-c4ccccc4)nc(-c4cc(-c5ccccc5)ccc4-c4ccccc4)n3)cc2)cc1 ; c1ccc(-c2ccc(-c3nc(-c4cccc(-c5ccccc5)c4)nc(-c4cc(-c5ccccc5)ccc4-c4ccccc4)n3)cc2)cc1 ; c1ccc(-c2ccc(-c3nc(-c4ccccc4-c4ccccc4)nc(-c4cc(-c5ccccc5)ccc4-c4ccccc4)n3)cc2)cc1 ; c1ccc(-c2cccc(-c3nc(-c4cccc(-c5ccccc5-c5ccccc5)c4)nc(-c4cc(-c5ccccc5)ccc4-c4ccccc4)n3)c2)cc1 ; c1ccc(-c2cccc(-c3nc(-c4ccccc4-c4ccccc4)nc(-c4cc(-c5ccccc5)ccc4-c4ccccc4)n3)c2)cc1]

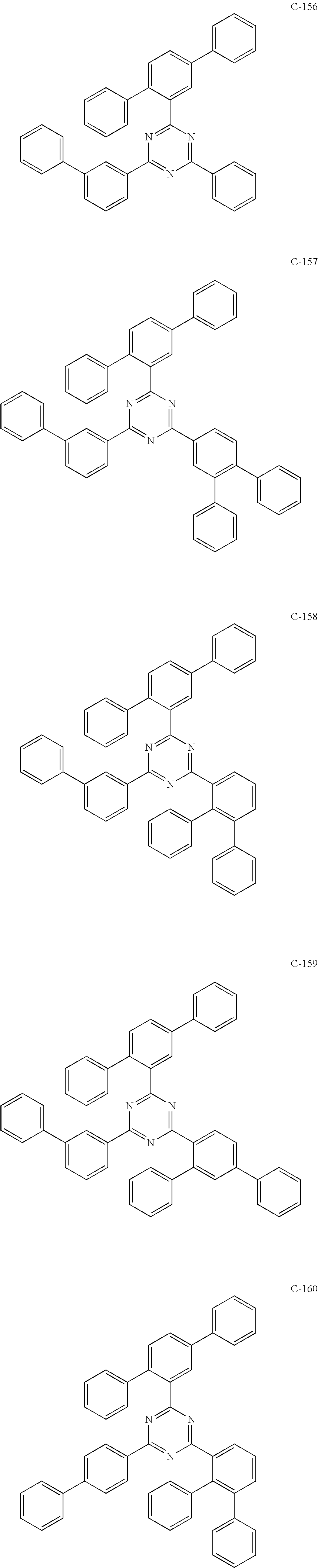 [SMILES: c1ccc(-c2cccc(-c3nc(-c4ccccc4)nc(-c4cc(-c5ccccc5)ccc4-c4ccccc4)n3)c2)cc1 ; c1ccc(-c2cccc(-c3nc(-c4ccc(-c5ccccc5)c(-c5ccccc5)c4)nc(-c4cc(-c5ccccc5)ccc4-c4ccccc4)n3)c2)cc1 ; c1ccc(-c2cccc(-c3nc(-c4cc(-c5ccccc5)ccc4-c4ccccc4)nc(-c4cccc(-c5ccccc5)c4-c4ccccc4)n3)c2)cc1 ; c1ccc(-c2cccc(-c3nc(-c4ccc(-c5ccccc5)cc4-c4ccccc4)nc(-c4cc(-c5ccccc5)ccc4-c4ccccc4)n3)c2)cc1 ; c1ccc(-c2ccc(-c3nc(-c4cc(-c5ccccc5)ccc4-c4ccccc4)nc(-c4cccc(-c5ccccc5)c4-c4ccccc4)n3)cc2)cc1]

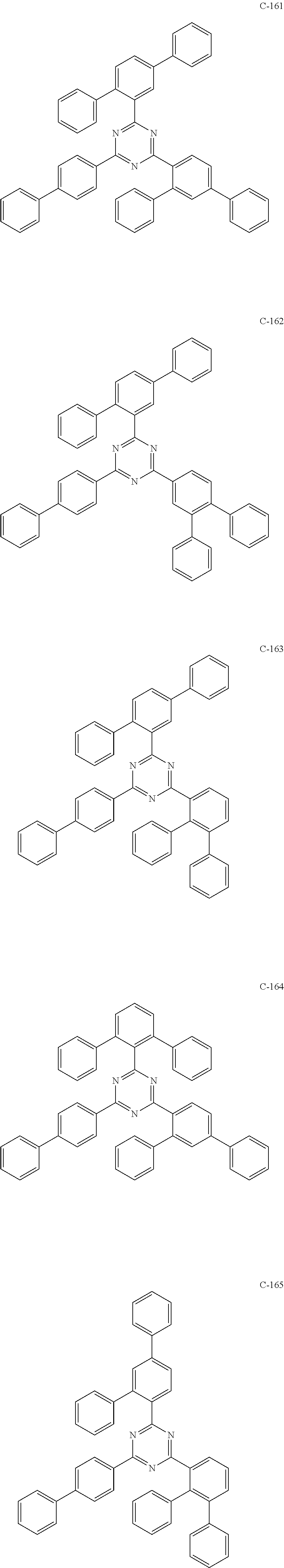 [SMILES: c1ccc(-c2ccc(-c3nc(-c4ccc(-c5ccccc5)cc4-c4ccccc4)nc(-c4cc(-c5ccccc5)ccc4-c4ccccc4)n3)cc2)cc1 ; c1ccc(-c2ccc(-c3nc(-c4ccc(-c5ccccc5)c(-c5ccccc5)c4)nc(-c4cc(-c5ccccc5)ccc4-c4ccccc4)n3)cc2)cc1 ; c1ccc(-c2ccc(-c3nc(-c4cc(-c5ccccc5)ccc4-c4ccccc4)nc(-c4cccc(-c5ccccc5)c4-c4ccccc4)n3)cc2)cc1 ; c1ccc(-c2ccc(-c3nc(-c4ccc(-c5ccccc5)cc4-c4ccccc4)nc(-c4c(-c5ccccc5)cccc4-c4ccccc4)n3)cc2)cc1 ; c1ccc(-c2ccc(-c3nc(-c4ccc(-c5ccccc5)cc4-c4ccccc4)nc(-c4cccc(-c5ccccc5)c4-c4ccccc4)n3)cc2)cc1]

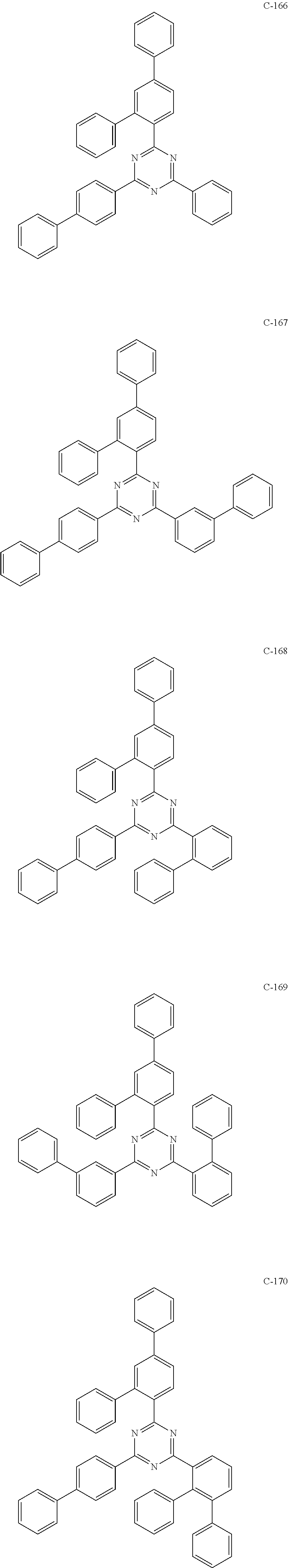 [SMILES: c1ccc(-c2ccc(-c3nc(-c4ccccc4)nc(-c4ccc(-c5ccccc5)cc4-c4ccccc4)n3)cc2)cc1 ; c1ccc(-c2ccc(-c3nc(-c4cccc(-c5ccccc5)c4)nc(-c4ccc(-c5ccccc5)cc4-c4ccccc4)n3)cc2)cc1 ; c1ccc(-c2ccc(-c3nc(-c4ccccc4-c4ccccc4)nc(-c4ccc(-c5ccccc5)cc4-c4ccccc4)n3)cc2)cc1 ; c1ccc(-c2cccc(-c3nc(-c4ccccc4-c4ccccc4)nc(-c4ccc(-c5ccccc5)cc4-c4ccccc4)n3)c2)cc1 ; c1ccc(-c2ccc(-c3nc(-c4ccc(-c5ccccc5)cc4-c4ccccc4)nc(-c4cccc(-c5ccccc5)c4-c4ccccc4)n3)cc2)cc1]

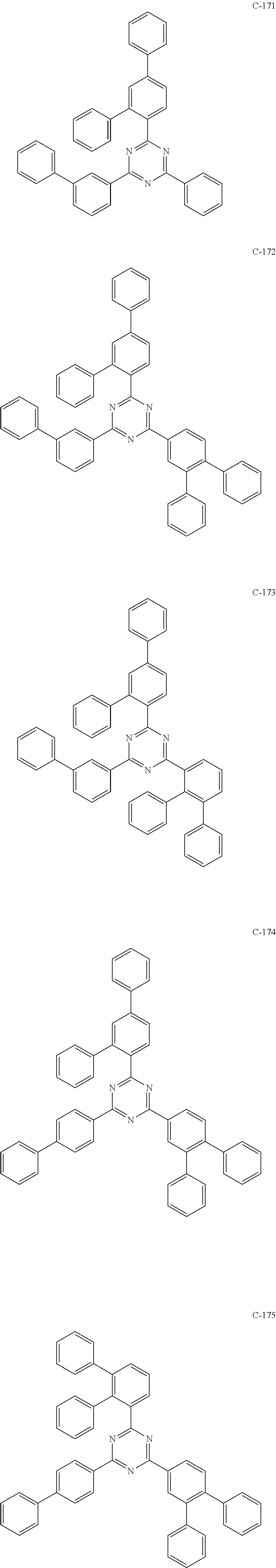 [SMILES: c1ccc(-c2cccc(-c3nc(-c4ccccc4)nc(-c4ccc(-c5ccccc5)cc4-c4ccccc4)n3)c2)cc1 ; c1ccc(-c2cccc(-c3nc(-c4ccc(-c5ccccc5)c(-c5ccccc5)c4)nc(-c4ccc(-c5ccccc5)cc4-c4ccccc4)n3)c2)cc1 ; c1ccc(-c2cccc(-c3nc(-c4ccc(-c5ccccc5)cc4-c4ccccc4)nc(-c4cccc(-c5ccccc5)c4-c4ccccc4)n3)c2)cc1 ; c1ccc(-c2ccc(-c3nc(-c4ccc(-c5ccccc5)c(-c5ccccc5)c4)nc(-c4ccc(-c5ccccc5)cc4-c4ccccc4)n3)cc2)cc1 ; c1ccc(-c2ccc(-c3nc(-c4ccc(-c5ccccc5)c(-c5ccccc5)c4)nc(-c4cccc(-c5ccccc5)c4-c4ccccc4)n3)cc2)cc1]

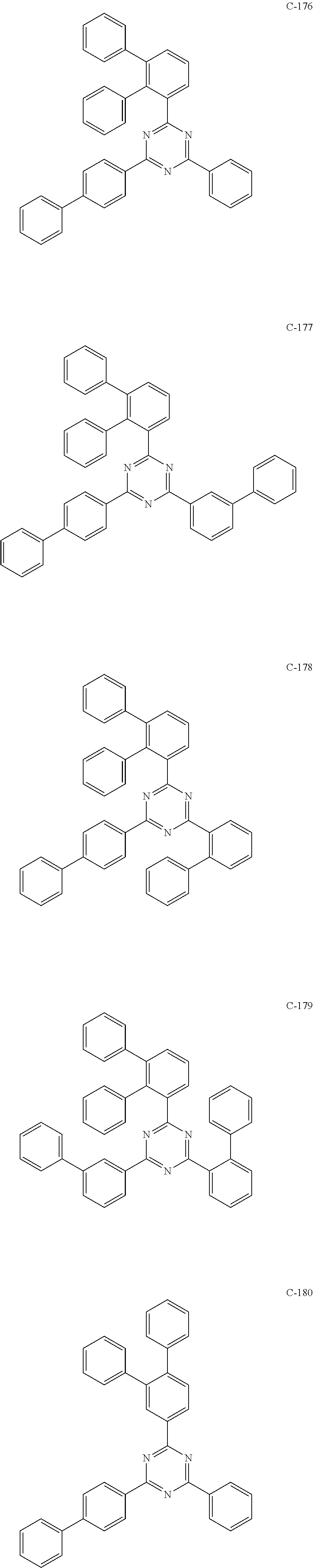 [SMILES: c1ccc(-c2ccc(-c3nc(-c4ccccc4)nc(-c4cccc(-c5ccccc5)c4-c4ccccc4)n3)cc2)cc1 ; c1ccc(-c2ccc(-c3nc(-c4cccc(-c5ccccc5)c4)nc(-c4cccc(-c5ccccc5)c4-c4ccccc4)n3)cc2)cc1 ; c1ccc(-c2ccc(-c3nc(-c4ccccc4-c4ccccc4)nc(-c4cccc(-c5ccccc5)c4-c4ccccc4)n3)cc2)cc1 ; c1ccc(-c2cccc(-c3nc(-c4ccccc4-c4ccccc4)nc(-c4cccc(-c5ccccc5)c4-c4ccccc4)n3)c2)cc1 ; c1ccc(-c2ccc(-c3nc(-c4ccccc4)nc(-c4ccc(-c5ccccc5)c(-c5ccccc5)c4)n3)cc2)cc1]

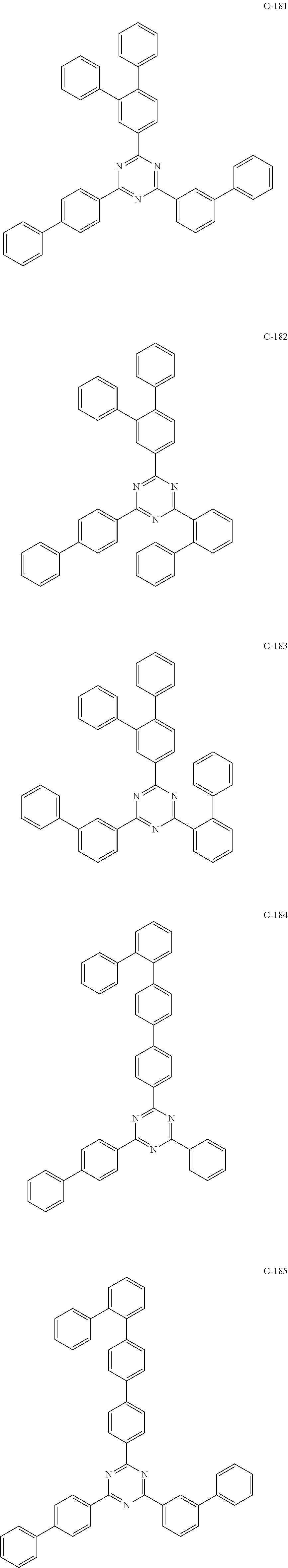 [SMILES: c1ccc(-c2ccc(-c3nc(-c4cccc(-c5ccccc5)c4)nc(-c4ccc(-c5ccccc5)c(-c5ccccc5)c4)n3)cc2)cc1 ; c1ccc(-c2ccc(-c3nc(-c4ccc(-c5ccccc5)c(-c5ccccc5)c4)nc(-c4ccccc4-c4ccccc4)n3)cc2)cc1 ; c1ccc(-c2cccc(-c3nc(-c4ccc(-c5ccccc5)c(-c5ccccc5)c4)nc(-c4ccccc4-c4ccccc4)n3)c2)cc1 ; c1ccc(-c2ccc(-c3nc(-c4ccccc4)nc(-c4ccc(-c5ccc(-c6ccccc6-c6ccccc6)cc5)cc4)n3)cc2)cc1 ; c1ccc(-c2ccc(-c3nc(-c4ccc(-c5ccc(-c6ccccc6-c6ccccc6)cc5)cc4)nc(-c4cccc(-c5ccccc5)c4)n3)cc2)cc1]

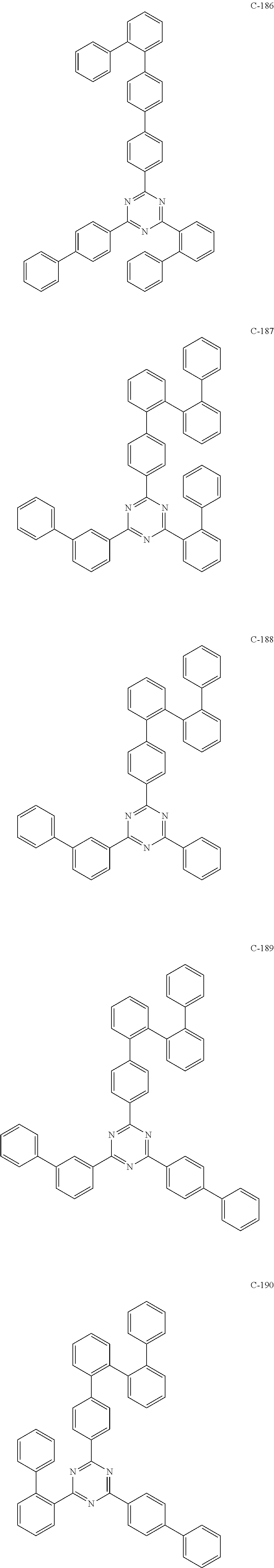

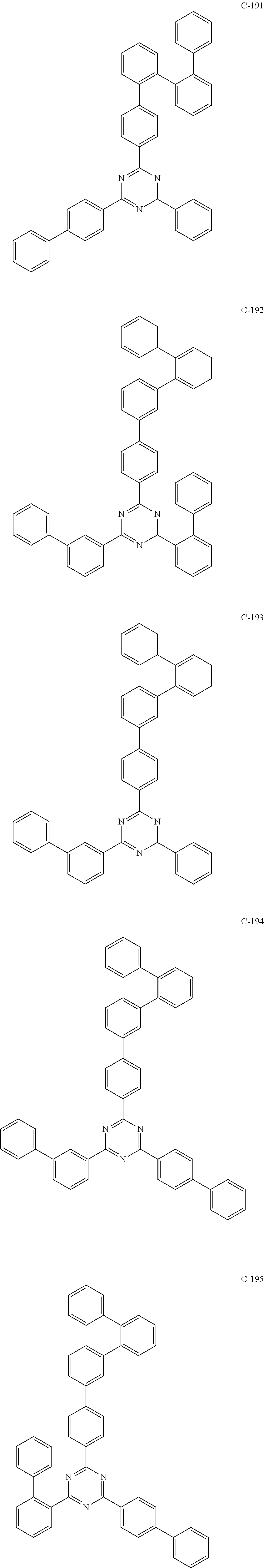 [SMILES: c1ccc(-c2ccc(-c3nc(-c4ccccc4)nc(-c4ccc(-c5ccccc5-c5ccccc5-c5ccccc5)cc4)n3)cc2)cc1 ; c1ccc(-c2cccc(-c3nc(-c4ccc(-c5cccc(-c6ccccc6-c6ccccc6)c5)cc4)nc(-c4ccccc4-c4ccccc4)n3)c2)cc1 ; c1ccc(-c2cccc(-c3nc(-c4ccccc4)nc(-c4ccc(-c5cccc(-c6ccccc6-c6ccccc6)c5)cc4)n3)c2)cc1 ; c1ccc(-c2ccc(-c3nc(-c4ccc(-c5cccc(-c6ccccc6-c6ccccc6)c5)cc4)nc(-c4cccc(-c5ccccc5)c4)n3)cc2)cc1 ; c1ccc(-c2ccc(-c3nc(-c4ccc(-c5cccc(-c6ccccc6-c6ccccc6)c5)cc4)nc(-c4ccccc4-c4ccccc4)n3)cc2)cc1]

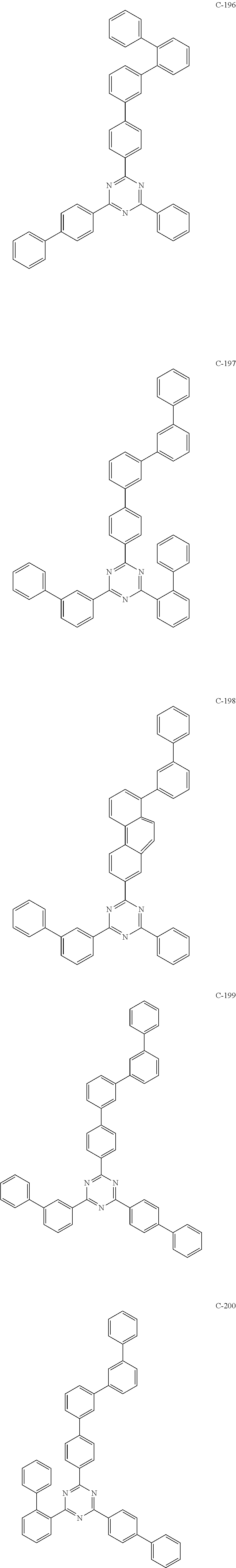 [SMILES: c1ccc(-c2ccc(-c3nc(-c4ccccc4)nc(-c4ccc(-c5cccc(-c6ccccc6-c6ccccc6)c5)cc4)n3)cc2)cc1 ; c1ccc(-c2cccc(-c3cccc(-c4ccc(-c5nc(-c6cccc(-c7ccccc7)c6)nc(-c6ccccc6-c6ccccc6)n5)cc4)c3)c2)cc1 ; c1ccc(-c2cccc(-c3nc(-c4ccccc4)nc(-c4ccc5c(ccc6c(-c7cccc(-c8ccccc8)c7)cccc65)c4)n3)c2)cc1 ; c1ccc(-c2ccc(-c3nc(-c4ccc(-c5cccc(-c6cccc(-c7ccccc7)c6)c5)cc4)nc(-c4cccc(-c5ccccc5)c4)n3)cc2)cc1 ; c1ccc(-c2ccc(-c3nc(-c4ccc(-c5cccc(-c6cccc(-c7ccccc7)c6)c5)cc4)nc(-c4ccccc4-c4ccccc4)n3)cc2)cc1]

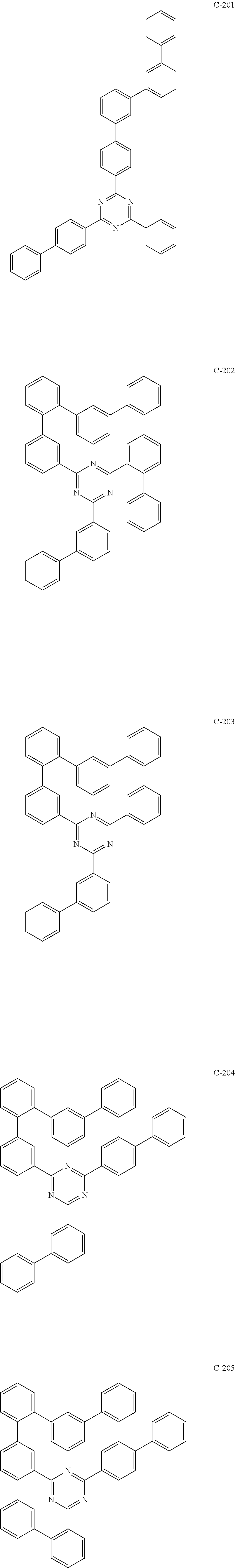 [SMILES: c1ccc(-c2ccc(-c3nc(-c4ccccc4)nc(-c4ccc(-c5cccc(-c6cccc(-c7ccccc7)c6)c5)cc4)n3)cc2)cc1 ; c1ccc(-c2cccc(-c3nc(-c4cccc(-c5ccccc5-c5cccc(-c6ccccc6)c5)c4)nc(-c4ccccc4-c4ccccc4)n3)c2)cc1 ; c1ccc(-c2cccc(-c3nc(-c4ccccc4)nc(-c4cccc(-c5ccccc5-c5cccc(-c6ccccc6)c5)c4)n3)c2)cc1 ; c1ccc(-c2ccc(-c3nc(-c4cccc(-c5ccccc5)c4)nc(-c4cccc(-c5ccccc5-c5cccc(-c6ccccc6)c5)c4)n3)cc2)cc1 ; c1ccc(-c2ccc(-c3nc(-c4cccc(-c5ccccc5-c5cccc(-c6ccccc6)c5)c4)nc(-c4ccccc4-c4ccccc4)n3)cc2)cc1]

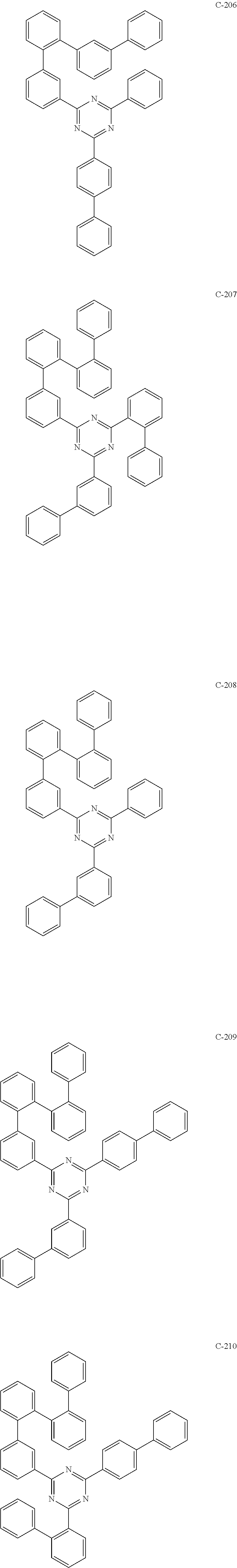 [SMILES: c1ccc(-c2ccc(-c3nc(-c4ccccc4)nc(-c4cccc(-c5ccccc5-c5cccc(-c6ccccc6)c5)c4)n3)cc2)cc1 ; c1ccc(-c2cccc(-c3nc(-c4cccc(-c5ccccc5-c5ccccc5-c5ccccc5)c4)nc(-c4ccccc4-c4ccccc4)n3)c2)cc1 ; c1ccc(-c2cccc(-c3nc(-c4ccccc4)nc(-c4cccc(-c5ccccc5-c5ccccc5-c5ccccc5)c4)n3)c2)cc1 ; c1ccc(-c2ccc(-c3nc(-c4cccc(-c5ccccc5)c4)nc(-c4cccc(-c5ccccc5-c5ccccc5-c5ccccc5)c4)n3)cc2)cc1 ; c1ccc(-c2ccc(-c3nc(-c4cccc(-c5ccccc5-c5ccccc5-c5ccccc5)c4)nc(-c4ccccc4-c4ccccc4)n3)cc2)cc1]

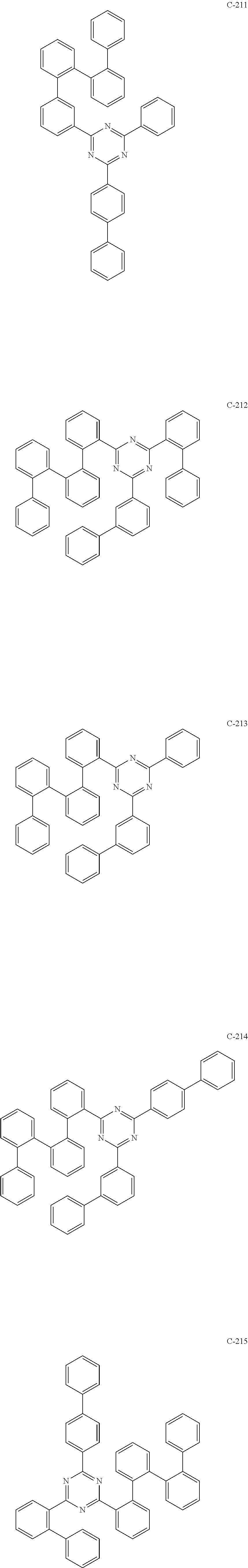 [SMILES: c1ccc(-c2ccc(-c3nc(-c4ccccc4)nc(-c4cccc(-c5ccccc5-c5ccccc5-c5ccccc5)c4)n3)cc2)cc1 ; c1ccc(-c2cccc(-c3nc(-c4ccccc4-c4ccccc4)nc(-c4ccccc4-c4ccccc4-c4ccccc4-c4ccccc4)n3)c2)cc1 ; c1ccc(-c2cccc(-c3nc(-c4ccccc4)nc(-c4ccccc4-c4ccccc4-c4ccccc4-c4ccccc4)n3)c2)cc1 ; c1ccc(-c2ccc(-c3nc(-c4cccc(-c5ccccc5)c4)nc(-c4ccccc4-c4ccccc4-c4ccccc4-c4ccccc4)n3)cc2)cc1 ; c1ccc(-c2ccc(-c3nc(-c4ccccc4-c4ccccc4)nc(-c4ccccc4-c4ccccc4-c4ccccc4-c4ccccc4)n3)cc2)cc1]

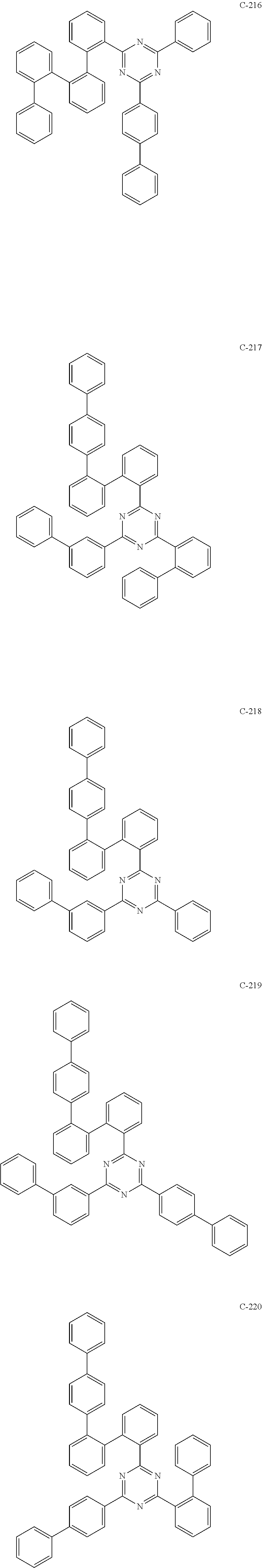 [SMILES: c1ccc(-c2ccc(-c3nc(-c4ccccc4)nc(-c4ccccc4-c4ccccc4-c4ccccc4-c4ccccc4)n3)cc2)cc1 ; c1ccc(-c2ccc(-c3ccccc3-c3ccccc3-c3nc(-c4cccc(-c5ccccc5)c4)nc(-c4ccccc4-c4ccccc4)n3)cc2)cc1 ; c1ccc(-c2ccc(-c3ccccc3-c3ccccc3-c3nc(-c4ccccc4)nc(-c4cccc(-c5ccccc5)c4)n3)cc2)cc1 ; c1ccc(-c2ccc(-c3nc(-c4cccc(-c5ccccc5)c4)nc(-c4ccccc4-c4ccccc4-c4ccc(-c5ccccc5)cc4)n3)cc2)cc1 ; c1ccc(-c2ccc(-c3nc(-c4ccccc4-c4ccccc4)nc(-c4ccccc4-c4ccccc4-c4ccc(-c5ccccc5)cc4)n3)cc2)cc1]

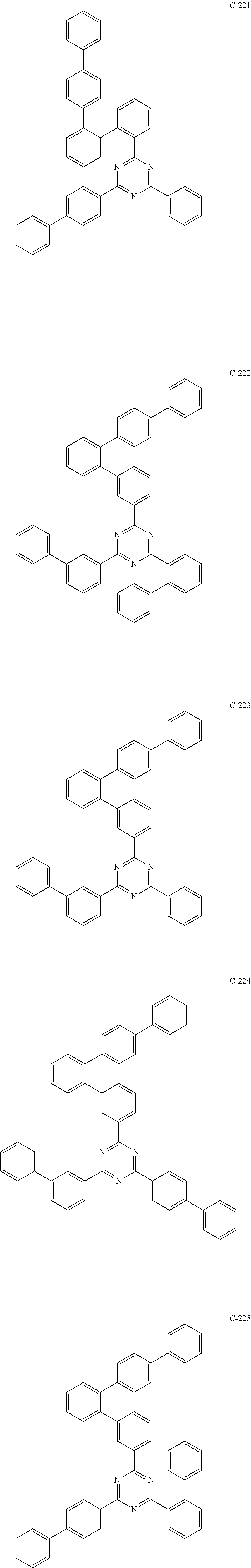 [SMILES: c1ccc(-c2ccc(-c3nc(-c4ccccc4)nc(-c4ccccc4-c4ccccc4-c4ccc(-c5ccccc5)cc4)n3)cc2)cc1 ; c1ccc(-c2ccc(-c3ccccc3-c3cccc(-c4nc(-c5cccc(-c6ccccc6)c5)nc(-c5ccccc5-c5ccccc5)n4)c3)cc2)cc1 ; c1ccc(-c2ccc(-c3ccccc3-c3cccc(-c4nc(-c5ccccc5)nc(-c5cccc(-c6ccccc6)c5)n4)c3)cc2)cc1 ; c1ccc(-c2ccc(-c3nc(-c4cccc(-c5ccccc5)c4)nc(-c4cccc(-c5ccccc5-c5ccc(-c6ccccc6)cc5)c4)n3)cc2)cc1 ; c1ccc(-c2ccc(-c3nc(-c4cccc(-c5ccccc5-c5ccc(-c6ccccc6)cc5)c4)nc(-c4ccccc4-c4ccccc4)n3)cc2)cc1]

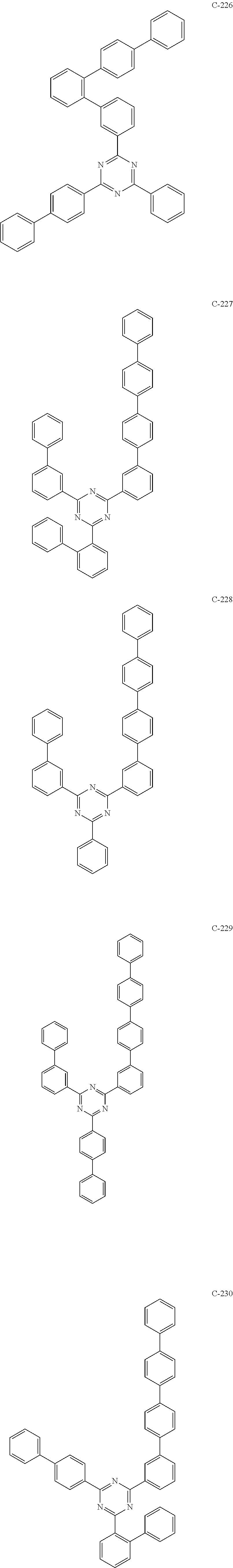 [SMILES: c1ccc(-c2ccc(-c3nc(-c4ccccc4)nc(-c4cccc(-c5ccccc5-c5ccc(-c6ccccc6)cc5)c4)n3)cc2)cc1 ; c1ccc(-c2ccc(-c3ccc(-c4cccc(-c5nc(-c6cccc(-c7ccccc7)c6)nc(-c6ccccc6-c6ccccc6)n5)c4)cc3)cc2)cc1 ; c1ccc(-c2ccc(-c3ccc(-c4cccc(-c5nc(-c6ccccc6)nc(-c6cccc(-c7ccccc7)c6)n5)c4)cc3)cc2)cc1 ; c1ccc(-c2ccc(-c3ccc(-c4cccc(-c5nc(-c6ccc(-c7ccccc7)cc6)nc(-c6cccc(-c7ccccc7)c6)n5)c4)cc3)cc2)cc1 ; c1ccc(-c2ccc(-c3ccc(-c4cccc(-c5nc(-c6ccc(-c7ccccc7)cc6)nc(-c6ccccc6-c6ccccc6)n5)c4)cc3)cc2)cc1]

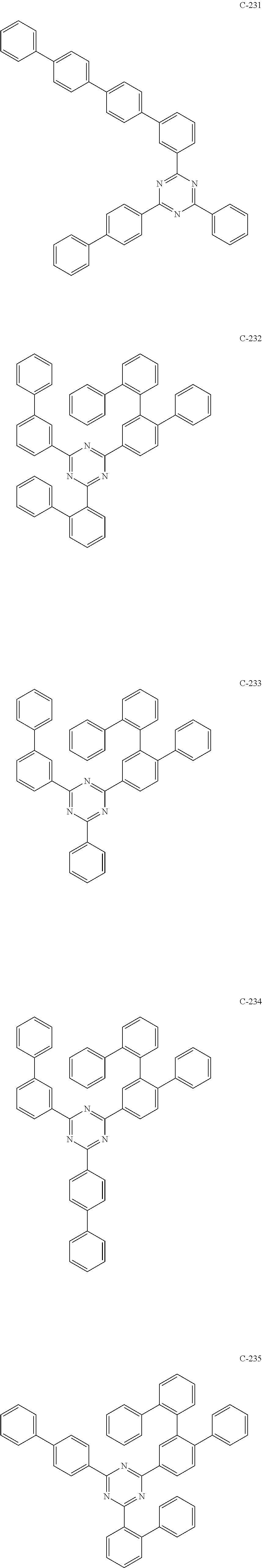 [SMILES: c1ccc(-c2ccc(-c3ccc(-c4cccc(-c5nc(-c6ccccc6)nc(-c6ccc(-c7ccccc7)cc6)n5)c4)cc3)cc2)cc1 ; c1ccc(-c2cccc(-c3nc(-c4ccc(-c5ccccc5)c(-c5ccccc5-c5ccccc5)c4)nc(-c4ccccc4-c4ccccc4)n3)c2)cc1 ; c1ccc(-c2cccc(-c3nc(-c4ccccc4)nc(-c4ccc(-c5ccccc5)c(-c5ccccc5-c5ccccc5)c4)n3)c2)cc1 ; c1ccc(-c2ccc(-c3nc(-c4cccc(-c5ccccc5)c4)nc(-c4ccc(-c5ccccc5)c(-c5ccccc5-c5ccccc5)c4)n3)cc2)cc1 ; c1ccc(-c2ccc(-c3nc(-c4ccc(-c5ccccc5)c(-c5ccccc5-c5ccccc5)c4)nc(-c4ccccc4-c4ccccc4)n3)cc2)cc1]

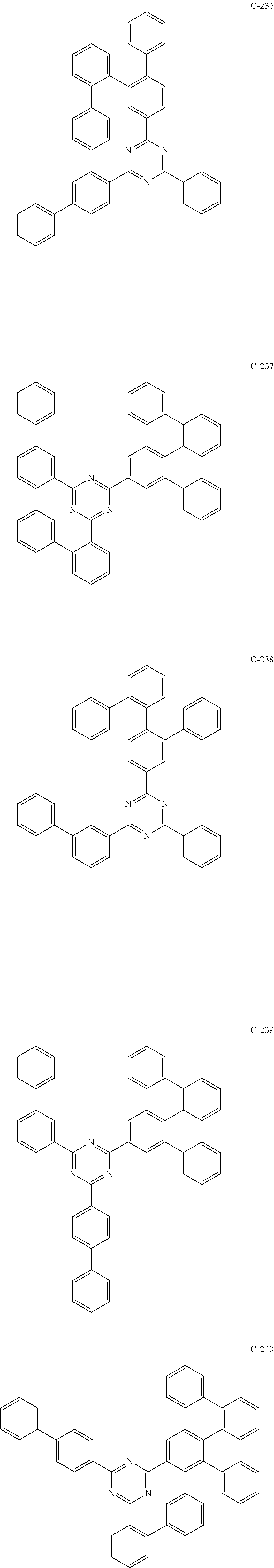 [SMILES: c1ccc(-c2ccc(-c3nc(-c4ccccc4)nc(-c4ccc(-c5ccccc5)c(-c5ccccc5-c5ccccc5)c4)n3)cc2)cc1 ; c1ccc(-c2cccc(-c3nc(-c4ccc(-c5ccccc5-c5ccccc5)c(-c5ccccc5)c4)nc(-c4ccccc4-c4ccccc4)n3)c2)cc1 ; c1ccc(-c2cccc(-c3nc(-c4ccccc4)nc(-c4ccc(-c5ccccc5-c5ccccc5)c(-c5ccccc5)c4)n3)c2)cc1 ; c1ccc(-c2ccc(-c3nc(-c4cccc(-c5ccccc5)c4)nc(-c4ccc(-c5ccccc5-c5ccccc5)c(-c5ccccc5)c4)n3)cc2)cc1 ; c1ccc(-c2ccc(-c3nc(-c4ccc(-c5ccccc5-c5ccccc5)c(-c5ccccc5)c4)nc(-c4ccccc4-c4ccccc4)n3)cc2)cc1]

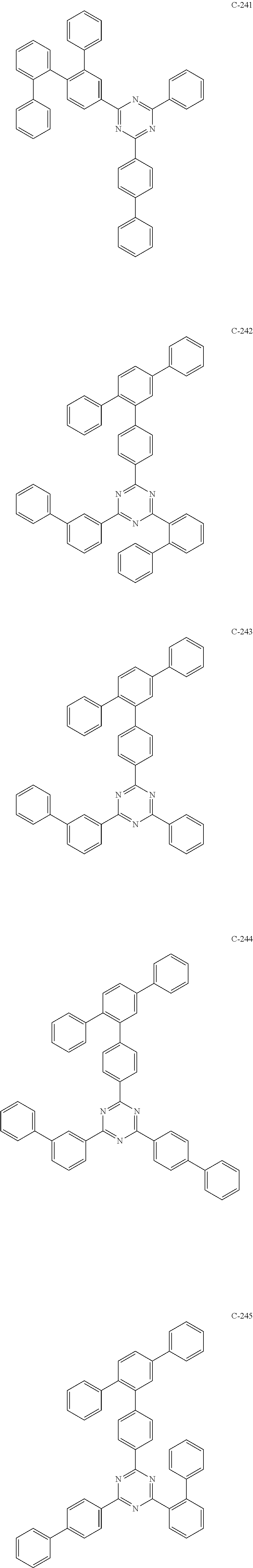 [SMILES: c1ccc(-c2ccc(-c3nc(-c4ccccc4)nc(-c4ccc(-c5ccccc5-c5ccccc5)c(-c5ccccc5)c4)n3)cc2)cc1 ; c1ccc(-c2cccc(-c3nc(-c4ccc(-c5cc(-c6ccccc6)ccc5-c5ccccc5)cc4)nc(-c4ccccc4-c4ccccc4)n3)c2)cc1 ; c1ccc(-c2cccc(-c3nc(-c4ccccc4)nc(-c4ccc(-c5cc(-c6ccccc6)ccc5-c5ccccc5)cc4)n3)c2)cc1 ; c1ccc(-c2ccc(-c3nc(-c4ccc(-c5cc(-c6ccccc6)ccc5-c5ccccc5)cc4)nc(-c4cccc(-c5ccccc5)c4)n3)cc2)cc1 ; c1ccc(-c2ccc(-c3nc(-c4ccc(-c5cc(-c6ccccc6)ccc5-c5ccccc5)cc4)nc(-c4ccccc4-c4ccccc4)n3)cc2)cc1]

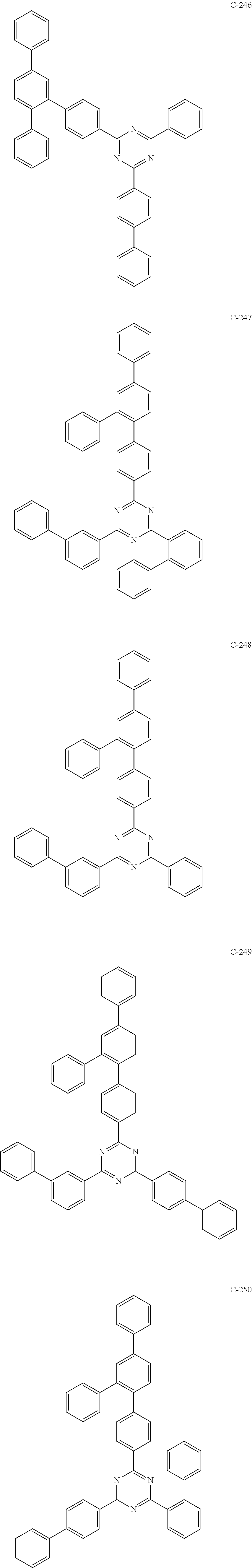 [SMILES: c1ccc(-c2ccc(-c3nc(-c4ccccc4)nc(-c4ccc(-c5cc(-c6ccccc6)ccc5-c5ccccc5)cc4)n3)cc2)cc1 ; c1ccc(-c2cccc(-c3nc(-c4ccc(-c5ccc(-c6ccccc6)cc5-c5ccccc5)cc4)nc(-c4ccccc4-c4ccccc4)n3)c2)cc1 ; c1ccc(-c2cccc(-c3nc(-c4ccccc4)nc(-c4ccc(-c5ccc(-c6ccccc6)cc5-c5ccccc5)cc4)n3)c2)cc1 ; c1ccc(-c2ccc(-c3nc(-c4ccc(-c5ccc(-c6ccccc6)cc5-c5ccccc5)cc4)nc(-c4cccc(-c5ccccc5)c4)n3)cc2)cc1 ; c1ccc(-c2ccc(-c3nc(-c4ccc(-c5ccc(-c6ccccc6)cc5-c5ccccc5)cc4)nc(-c4ccccc4-c4ccccc4)n3)cc2)cc1]

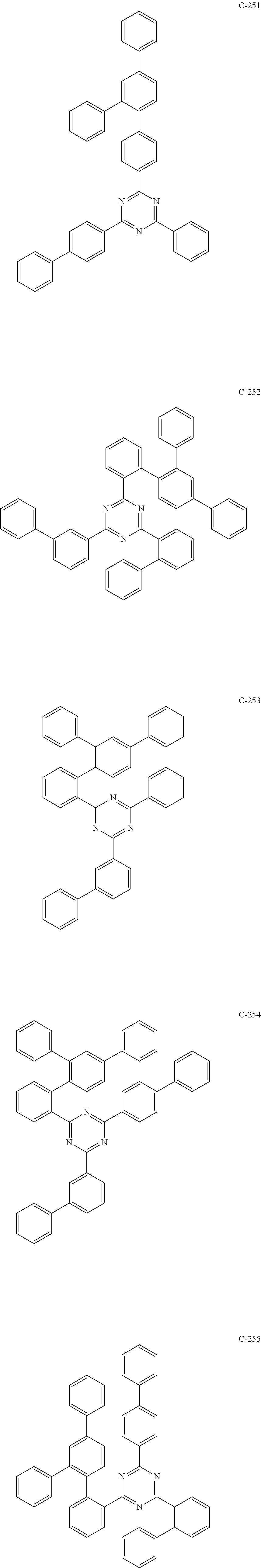 [SMILES: c1ccc(-c2ccc(-c3nc(-c4ccccc4)nc(-c4ccc(-c5ccc(-c6ccccc6)cc5-c5ccccc5)cc4)n3)cc2)cc1 ; c1ccc(-c2cccc(-c3nc(-c4ccccc4-c4ccccc4)nc(-c4ccccc4-c4ccc(-c5ccccc5)cc4-c4ccccc4)n3)c2)cc1 ; c1ccc(-c2cccc(-c3nc(-c4ccccc4)nc(-c4ccccc4-c4ccc(-c5ccccc5)cc4-c4ccccc4)n3)c2)cc1 ; c1ccc(-c2ccc(-c3nc(-c4cccc(-c5ccccc5)c4)nc(-c4ccccc4-c4ccc(-c5ccccc5)cc4-c4ccccc4)n3)cc2)cc1 ; c1ccc(-c2ccc(-c3nc(-c4ccccc4-c4ccccc4)nc(-c4ccccc4-c4ccc(-c5ccccc5)cc4-c4ccccc4)n3)cc2)cc1]

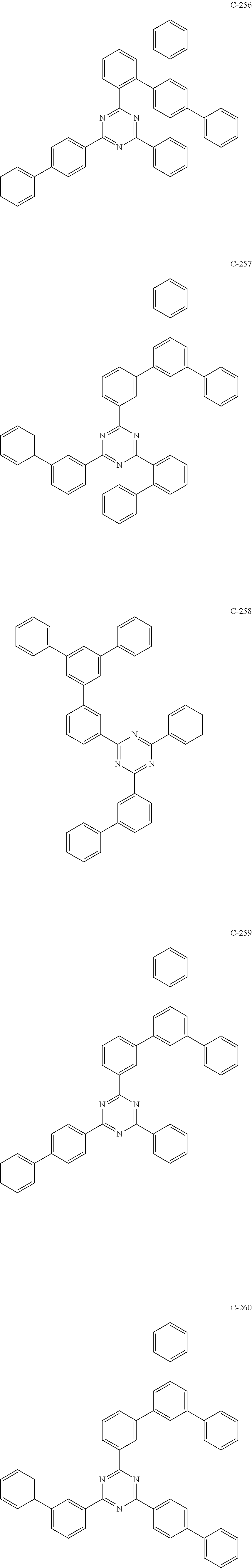 [SMILES: c1ccc(-c2ccc(-c3nc(-c4ccccc4)nc(-c4ccccc4-c4ccc(-c5ccccc5)cc4-c4ccccc4)n3)cc2)cc1 ; c1ccc(-c2cc(-c3ccccc3)cc(-c3cccc(-c4nc(-c5cccc(-c6ccccc6)c5)nc(-c5ccccc5-c5ccccc5)n4)c3)c2)cc1 ; c1ccc(-c2cc(-c3ccccc3)cc(-c3cccc(-c4nc(-c5ccccc5)nc(-c5cccc(-c6ccccc6)c5)n4)c3)c2)cc1 ; c1ccc(-c2ccc(-c3nc(-c4ccccc4)nc(-c4cccc(-c5cc(-c6ccccc6)cc(-c6ccccc6)c5)c4)n3)cc2)cc1 ; c1ccc(-c2ccc(-c3nc(-c4cccc(-c5ccccc5)c4)nc(-c4cccc(-c5cc(-c6ccccc6)cc(-c6ccccc6)c5)c4)n3)cc2)cc1]

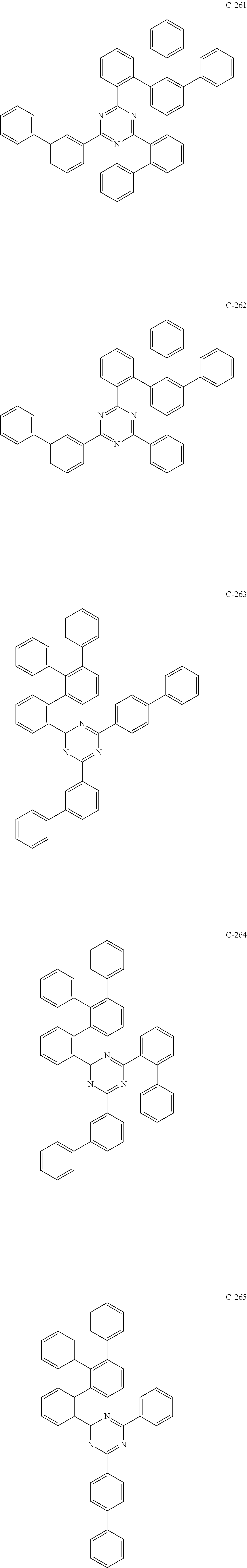 [SMILES: c1ccc(-c2cccc(-c3nc(-c4ccccc4-c4ccccc4)nc(-c4ccccc4-c4cccc(-c5ccccc5)c4-c4ccccc4)n3)c2)cc1 ; c1ccc(-c2cccc(-c3nc(-c4ccccc4)nc(-c4ccccc4-c4cccc(-c5ccccc5)c4-c4ccccc4)n3)c2)cc1 ; c1ccc(-c2ccc(-c3nc(-c4cccc(-c5ccccc5)c4)nc(-c4ccccc4-c4cccc(-c5ccccc5)c4-c4ccccc4)n3)cc2)cc1 ; c1ccc(-c2cccc(-c3nc(-c4ccccc4-c4ccccc4)nc(-c4ccccc4-c4cccc(-c5ccccc5)c4-c4ccccc4)n3)c2)cc1 ; c1ccc(-c2ccc(-c3nc(-c4ccccc4)nc(-c4ccccc4-c4cccc(-c5ccccc5)c4-c4ccccc4)n3)cc2)cc1]

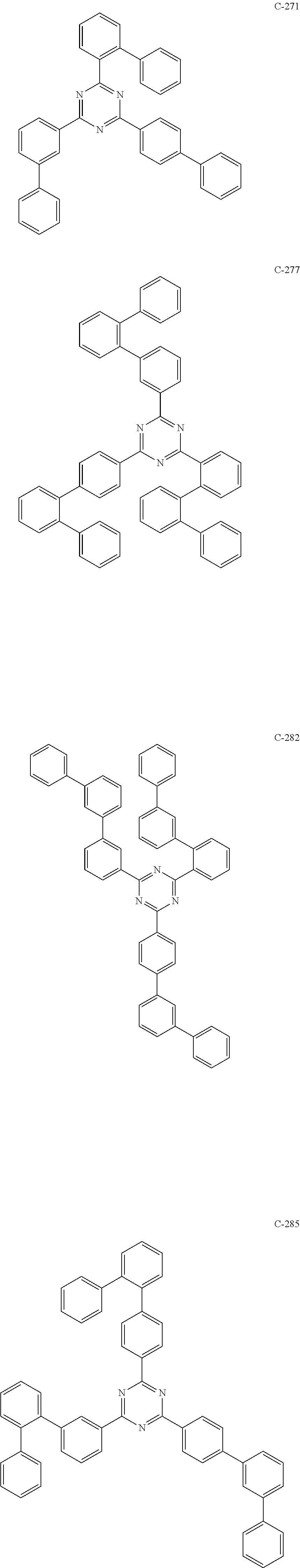 [SMILES: c1ccc(-c2ccc(-c3nc(-c4cccc(-c5ccccc5)c4)nc(-c4ccccc4-c4ccccc4)n3)cc2)cc1 ; c1ccc(-c2ccccc2-c2ccc(-c3nc(-c4cccc(-c5ccccc5-c5ccccc5)c4)nc(-c4ccccc4-c4ccccc4-c4ccccc4)n3)cc2)cc1 ; c1ccc(-c2cccc(-c3ccc(-c4nc(-c5cccc(-c6cccc(-c7ccccc7)c6)c5)nc(-c5ccccc5-c5cccc(-c6ccccc6)c5)n4)cc3)c2)cc1 ; c1ccc(-c2cccc(-c3ccc(-c4nc(-c5ccc(-c6ccccc6-c6ccccc6)cc5)nc(-c5cccc(-c6ccccc6-c6ccccc6)c5)n4)cc3)c2)cc1]

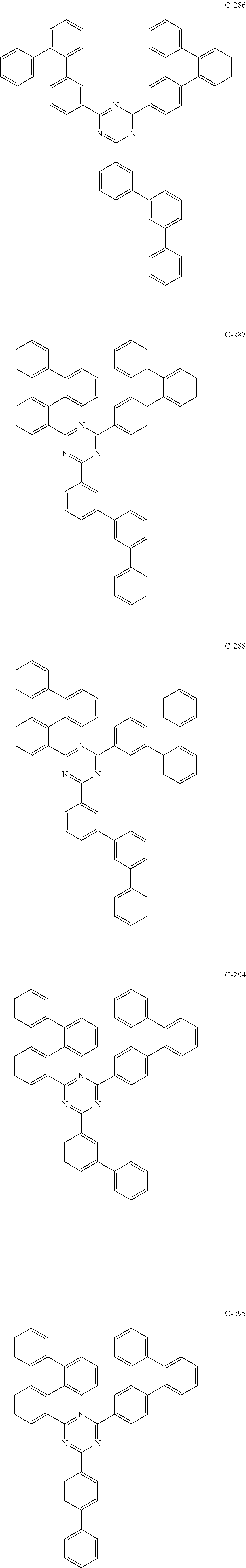 [SMILES: c1ccc(-c2cccc(-c3cccc(-c4nc(-c5ccc(-c6ccccc6-c6ccccc6)cc5)nc(-c5cccc(-c6ccccc6-c6ccccc6)c5)n4)c3)c2)cc1 ; c1ccc(-c2cccc(-c3cccc(-c4nc(-c5ccc(-c6ccccc6-c6ccccc6)cc5)nc(-c5ccccc5-c5ccccc5-c5ccccc5)n4)c3)c2)cc1 ; c1ccc(-c2cccc(-c3cccc(-c4nc(-c5cccc(-c6ccccc6-c6ccccc6)c5)nc(-c5ccccc5-c5ccccc5-c5ccccc5)n4)c3)c2)cc1 ; c1ccc(-c2cccc(-c3nc(-c4ccc(-c5ccccc5-c5ccccc5)cc4)nc(-c4ccccc4-c4ccccc4-c4ccccc4)n3)c2)cc1 ; c1ccc(-c2ccc(-c3nc(-c4ccc(-c5ccccc5-c5ccccc5)cc4)nc(-c4ccccc4-c4ccccc4-c4ccccc4)n3)cc2)cc1]

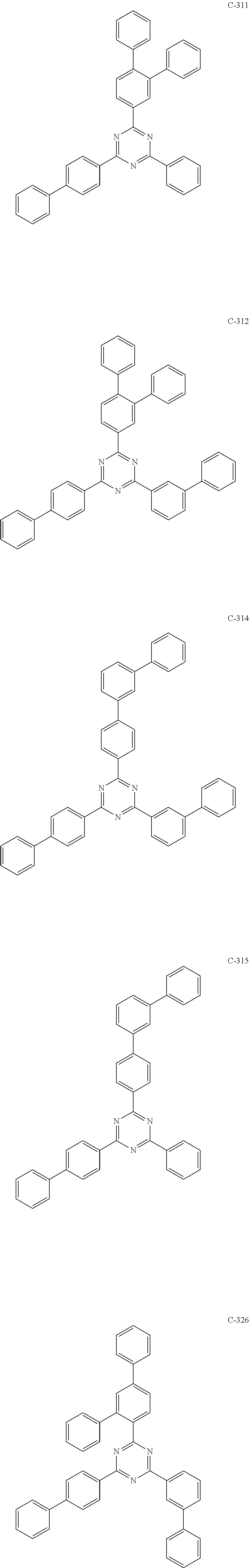 [SMILES: c1ccc(-c2ccc(-c3nc(-c4ccccc4)nc(-c4ccc(-c5ccccc5)c(-c5ccccc5)c4)n3)cc2)cc1 ; c1ccc(-c2ccc(-c3nc(-c4cccc(-c5ccccc5)c4)nc(-c4ccc(-c5ccccc5)c(-c5ccccc5)c4)n3)cc2)cc1 ; c1ccc(-c2ccc(-c3nc(-c4ccc(-c5cccc(-c6ccccc6)c5)cc4)nc(-c4cccc(-c5ccccc5)c4)n3)cc2)cc1 ; c1ccc(-c2ccc(-c3nc(-c4ccccc4)nc(-c4ccc(-c5cccc(-c6ccccc6)c5)cc4)n3)cc2)cc1 ; c1ccc(-c2ccc(-c3nc(-c4cccc(-c5ccccc5)c4)nc(-c4ccc(-c5ccccc5)cc4-c4ccccc4)n3)cc2)cc1]

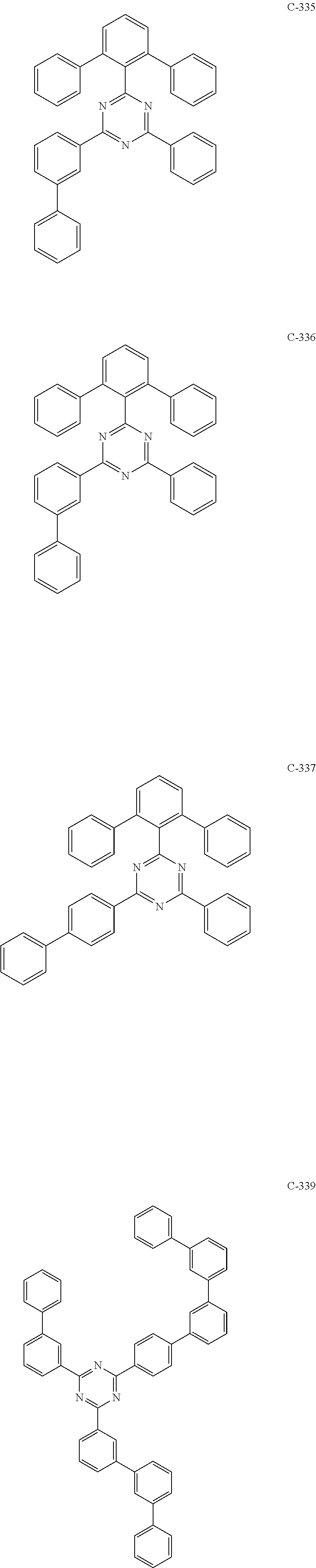 [SMILES: c1ccc(-c2cccc(-c3nc(-c4ccccc4)nc(-c4c(-c5ccccc5)cccc4-c4ccccc4)n3)c2)cc1 ; c1ccc(-c2cccc(-c3nc(-c4ccccc4)nc(-c4c(-c5ccccc5)cccc4-c4ccccc4)n3)c2)cc1 ; c1ccc(-c2ccc(-c3nc(-c4ccccc4)nc(-c4c(-c5ccccc5)cccc4-c4ccccc4)n3)cc2)cc1 ; c1ccc(-c2cccc(-c3cccc(-c4ccc(-c5nc(-c6cccc(-c7ccccc7)c6)nc(-c6cccc(-c7cccc(-c8ccccc8)c7)c6)n5)cc4)c3)c2)cc1]

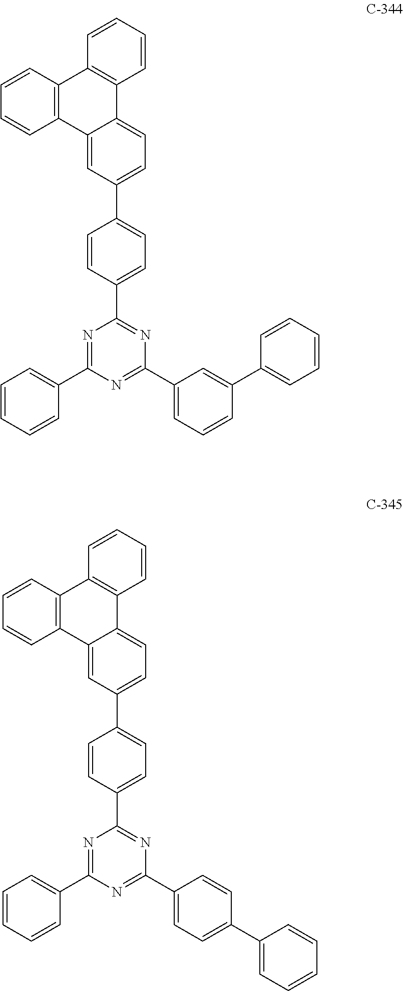 [SMILES: c1ccc(-c2cccc(-c3nc(-c4ccccc4)nc(-c4ccc(-c5ccc6c7ccccc7c7ccccc7c6c5)cc4)n3)c2)cc1 ; c1ccc(-c2ccc(-c3nc(-c4ccccc4)nc(-c4ccc(-c5ccc6c7ccccc7c7ccccc7c6c5)cc4)n3)cc2)cc1]

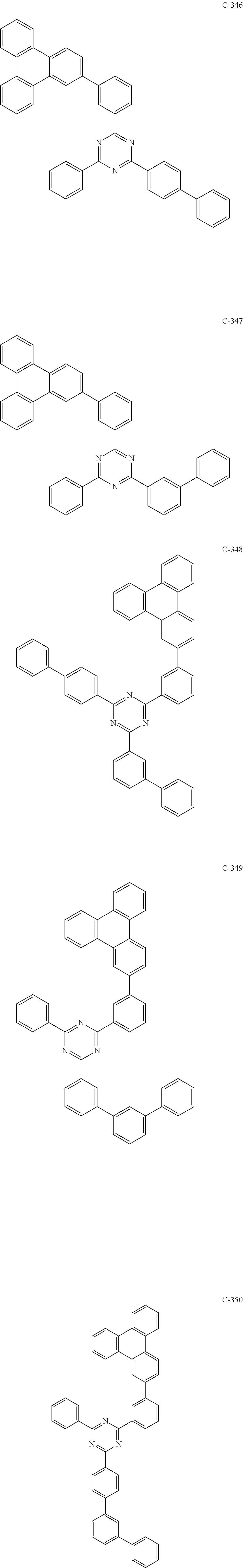 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2cccc(-c3nc(-c4ccccc4)nc(-c4cccc(-c5ccc6c7ccccc7c7ccccc7c6c5)c4)n3)c2)cc1 ; c1ccc(-c2ccc(-c3nc(-c4cccc(-c5ccccc5)c4)nc(-c4cccc(-c5ccc6c7ccccc7c7ccccc7c6c5)c4)n3)cc2)cc1 ; c1ccc(-c2cccc(-c3cccc(-c4nc(-c5ccccc5)nc(-c5cccc(-c6ccc7c8ccccc8c8ccccc8c7c6)c5)n4)c3)c2)cc1 ; c1ccc(-c2cccc(-c3ccc(-c4nc(-c5ccccc5)nc(-c5cccc(-c6ccc7c8ccccc8c8ccccc8c7c6)c5)n4)cc3)c2)cc1]

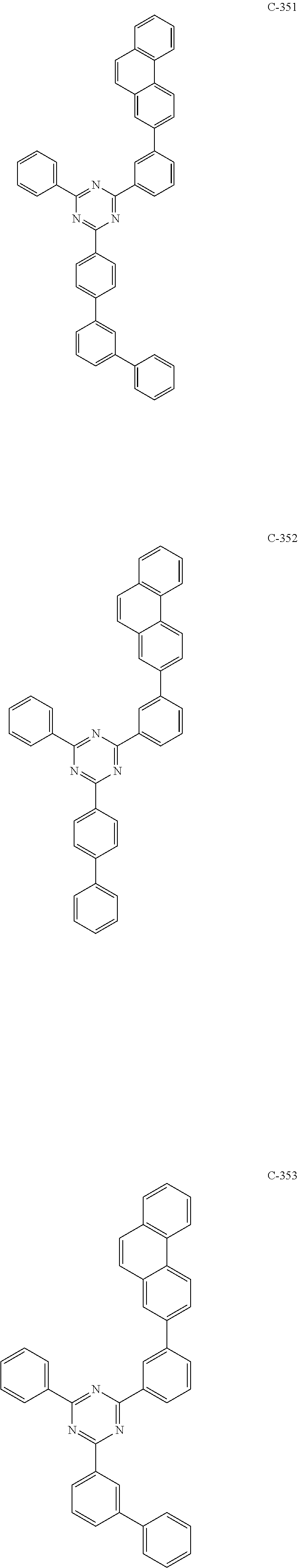 [SMILES: c1ccc(-c2cccc(-c3ccc(-c4nc(-c5ccccc5)nc(-c5cccc(-c6ccc7c(ccc8ccccc87)c6)c5)n4)cc3)c2)cc1 ; c1ccc(-c2ccc(-c3nc(-c4ccccc4)nc(-c4cccc(-c5ccc6c(ccc7ccccc76)c5)c4)n3)cc2)cc1 ; c1ccc(-c2cccc(-c3nc(-c4ccccc4)nc(-c4cccc(-c5ccc6c(ccc7ccccc76)c5)c4)n3)c2)cc1]

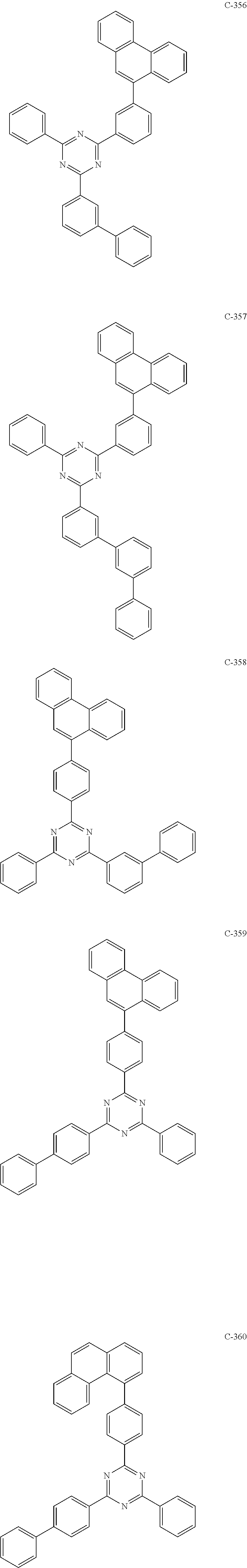 [SMILES: c1ccc(-c2cccc(-c3nc(-c4ccccc4)nc(-c4cccc(-c5cc6ccccc6c6ccccc56)c4)n3)c2)cc1 ; c1ccc(-c2cccc(-c3cccc(-c4nc(-c5ccccc5)nc(-c5cccc(-c6cc7ccccc7c7ccccc67)c5)n4)c3)c2)cc1 ; c1ccc(-c2cccc(-c3nc(-c4ccccc4)nc(-c4ccc(-c5cc6ccccc6c6ccccc56)cc4)n3)c2)cc1 ; c1ccc(-c2ccc(-c3nc(-c4ccccc4)nc(-c4ccc(-c5cc6ccccc6c6ccccc56)cc4)n3)cc2)cc1 ; c1ccc(-c2ccc(-c3nc(-c4ccccc4)nc(-c4ccc(-c5cccc6ccc7ccccc7c56)cc4)n3)cc2)cc1]

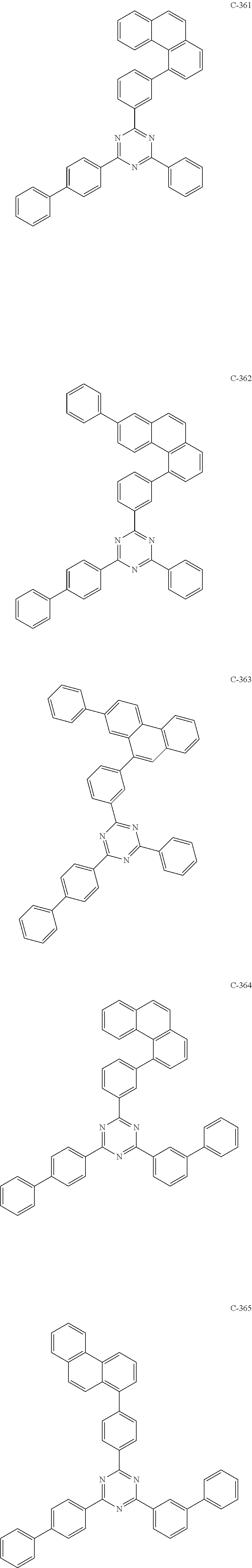 [SMILES: c1ccc(-c2ccc(-c3nc(-c4ccccc4)nc(-c4cccc(-c5cccc6ccc7ccccc7c56)c4)n3)cc2)cc1 ; c1ccc(-c2ccc(-c3nc(-c4ccccc4)nc(-c4cccc(-c5cccc6ccc7cc(-c8ccccc8)ccc7c56)c4)n3)cc2)cc1 ; c1ccc(-c2ccc(-c3nc(-c4ccccc4)nc(-c4cccc(-c5cc6ccccc6c6ccc(-c7ccccc7)cc56)c4)n3)cc2)cc1 ; c1ccc(-c2ccc(-c3nc(-c4cccc(-c5ccccc5)c4)nc(-c4cccc(-c5cccc6ccc7ccccc7c56)c4)n3)cc2)cc1 ; c1ccc(-c2ccc(-c3nc(-c4ccc(-c5cccc6c5ccc5ccccc56)cc4)nc(-c4cccc(-c5ccccc5)c4)n3)cc2)cc1]

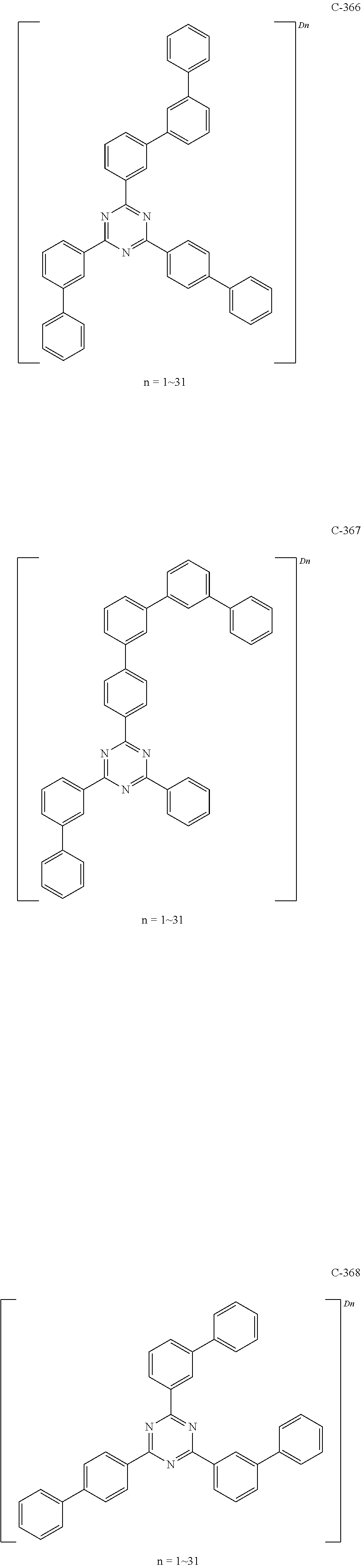

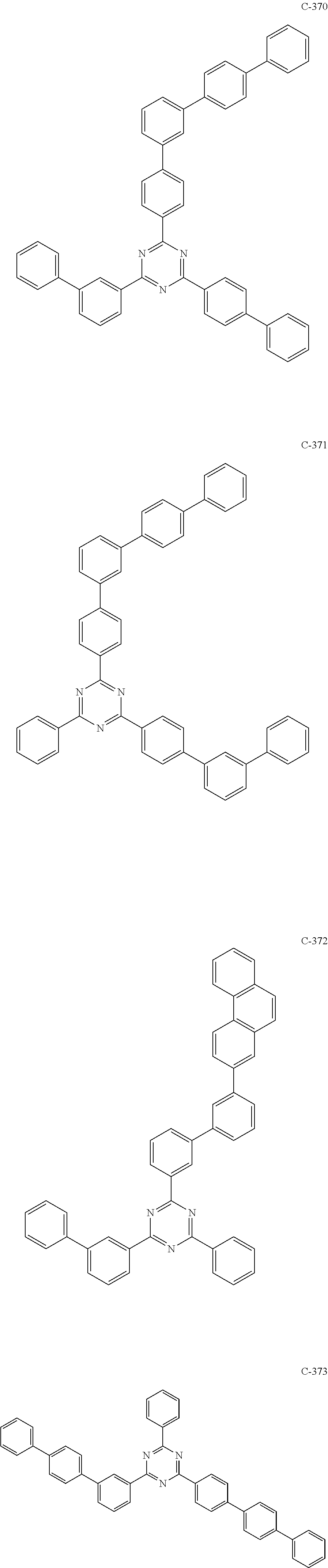 [SMILES: c1ccc(-c2ccc(-c3cccc(-c4ccc(-c5nc(-c6ccc(-c7ccccc7)cc6)nc(-c6cccc(-c7ccccc7)c6)n5)cc4)c3)cc2)cc1 ; c1ccc(-c2ccc(-c3cccc(-c4ccc(-c5nc(-c6ccccc6)nc(-c6ccc(-c7cccc(-c8ccccc8)c7)cc6)n5)cc4)c3)cc2)cc1 ; c1ccc(-c2cccc(-c3nc(-c4ccccc4)nc(-c4cccc(-c5cccc(-c6ccc7c(ccc8ccccc87)c6)c5)c4)n3)c2)cc1 ; c1ccc(-c2ccc(-c3ccc(-c4nc(-c5ccccc5)nc(-c5cccc(-c6ccc(-c7ccccc7)cc6)c5)n4)cc3)cc2)cc1]

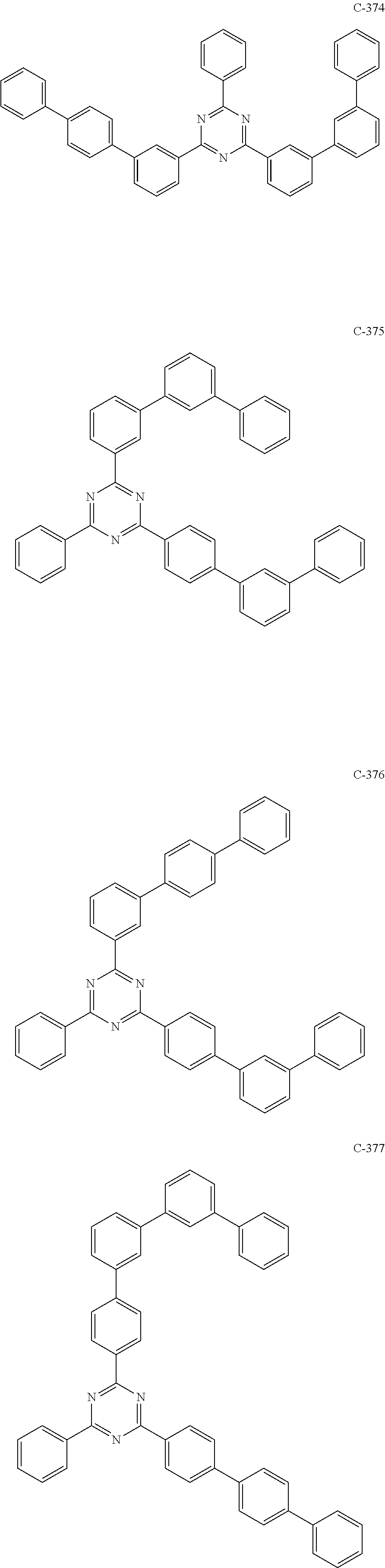 [SMILES: c1ccc(-c2ccc(-c3cccc(-c4nc(-c5ccccc5)nc(-c5cccc(-c6cccc(-c7ccccc7)c6)c5)n4)c3)cc2)cc1 ; c1ccc(-c2cccc(-c3ccc(-c4nc(-c5ccccc5)nc(-c5cccc(-c6cccc(-c7ccccc7)c6)c5)n4)cc3)c2)cc1 ; c1ccc(-c2ccc(-c3cccc(-c4nc(-c5ccccc5)nc(-c5ccc(-c6cccc(-c7ccccc7)c6)cc5)n4)c3)cc2)cc1 ; c1ccc(-c2ccc(-c3ccc(-c4nc(-c5ccccc5)nc(-c5ccc(-c6cccc(-c7cccc(-c8ccccc8)c7)c6)cc5)n4)cc3)cc2)cc1]

In the above compounds, Dn means that n number of hydrogen atoms are replaced with deuterium.

The present disclosure provides an organic electroluminescent material comprising the organic electroluminescent compound represented by formula 1-1 and an organic electroluminescent device comprising the same.

The organic electroluminescent material may consist of the organic electroluminescent compound according to the present disclosure only, or may further comprise a conventional material included in an organic electroluminescent material.

The organic electroluminescent compound of formula 1-1 according to the present disclosure may be included in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer, preferably as a host material of a light-emitting layer.

A plurality of host materials according to the present disclosure comprise a first host material and a second host material, wherein the first host material comprises at least one compound represented by formula 1, and the second host material comprises at least one compound represented by formula 2, wherein at least one of the first host compound represented by formula 1 and the second host compound represented by formula 2 contains deuterium.

In formula 1, X₁ to X₃ each independently represent N or CRₐ; with the proviso that at least two of X₁ to X₃ are N. According to one embodiment of the present disclosure, all of X₁ to X₃ may represent N.

In formula 1, Rₐ represents hydrogen or deuterium.

In formula 1, Ar₁ to Ar₃ each independently represent a (C6-C30)aryl unsubstituted or substituted with at least one of deuterium and a (C6-C30)aryl(s). According to one embodiment of the present disclosure, Ar₁ to Ar₃ each independently represent a (C6-C28)aryl, wherein the (C6-C28)aryl may be unsubstituted or substituted with at least one of deuterium and a (C6-C25)aryl(s) unsubstituted or substituted with a (C6-C12)aryl(s). According to another embodiment of the present disclosure, Ar₁ to Ar₃ each independently represent a (C6-C25)aryl, wherein the (C6-C25)aryl may be unsubstituted or substituted with at least one of deuterium and a (C6-C20)aryl(s) unsubstituted or substituted with a (C6-C10)aryl(s). For example, Ar₁ to Ar₃ may each independently be a phenyl unsubstituted or substituted with a triphenylenyl(s), a phenanthrenyl(s), or a phenylphenanthrenyl(s); a biphenyl; a terphenyl; a quaterphenyl, etc, which may be further substituted with deuterium.

According to another embodiment of the present disclosure, Ar₁ to Ar₃ may each independently represent a phenyl unsubstituted or substituted with deuterium, a biphenyl unsubstituted or substituted with deuterium, a terphenyl unsubstituted or substituted with deuterium, a quaterphenyl unsubstituted or substituted with deuterium, a naphthyl unsubstituted or substituted with deuterium, a phenylnaphthyl unsubstituted or substituted with deuterium, a naphthylphenyl unsubstituted or substituted with deuterium, a triphenylenyl unsubstituted or substituted with deuterium, a phenanthrenyl unsubstituted or substituted with deuterium, or a combination thereof.

According to another embodiment of the present disclosure, Ar₁ to Ar₃ may be all different from each other.

According to one embodiment of the present disclosure, when the compound of formula 1 contains deuterium, the deuterium substitution rate thereof may be about 30% to about 100%, preferably about 50% to about 100%, and more preferably about 70% to about 100%. The compound of formula 1 having said deuterium substitution rate may increase the bond dissociation energy due to deuteration to increase the stability of the compound, and an organic electroluminescent device comprising the compound may exhibit improved lifespan properties.

Hereinafter, the compound represented by formula 2 will be described in more detail.

In formula 2, A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl. According to one embodiment of the present disclosure, A₁ and A₂ each independently represent a (C6-C25)aryl unsubstituted or substituted with at least one of deuterium, a (C6-C30)aryl(s), and a (6- to 25-membered)heteroaryl(s); a dibenzofuranyl unsubstituted or substituted with at least one of deuterium and a (C6-C20)aryl(s); a dibenzothiophenyl unsubstituted or substituted with at least one of deuterium and a (C6-C20)aryl(s); or a carbazolyl unsubstituted or substituted with at least one of deuterium and a (C6-C25)aryl(s). According to another embodiment of the present disclosure, A₁ and A₂ may each independently represent a (C6-C20)aryl unsubstituted or substituted with at least one of deuterium, a (C6-C20)aryl(s), and a (6- to 15-membered)heteroaryl(s); a dibenzofuranyl unsubstituted or substituted with at least one of deuterium and a (C6-C10)aryl(s); a dibenzothiophenyl unsubstituted or substituted with at least one of deuterium and a (C6-C10)aryl(s); or a carbazolyl unsubstituted or substituted with at least one of deuterium and a (C6-C15)aryl(s). For example, A₁ and A₂ may each independently be a phenyl unsubstituted or substituted with at least one of a naphthyl(s), a triphenylenyl(s), a dibenzofuranyl(s), and a dibenzothiophenyl(s); a biphenyl; a terphenyl; a naphthyl unsubstituted or substituted with a phenyl(s); a triphenylenyl; a dibenzofuranyl unsubstituted or substituted with a phenyl(s); a dibenzothiophenyl unsubstituted or substituted with a phenyl(s); a carbazolyl unsubstituted or substituted with a phenyl(s) or naphthyl(s), etc., which may be further substituted with deuterium.

According to another embodiment of the present disclosure, A₁ and A₂ may each independently represent a phenyl unsubstituted or substituted with deuterium, a biphenyl unsubstituted or substituted with deuterium, a terphenyl unsubstituted or substituted with deuterium, a naphthyl unsubstituted or substituted with deuterium, a fluorenyl unsubstituted or substituted with at least one of deuterium, a (C1-C30)alkyl(s), and a (C6-C30)aryl(s), a benzofluorenyl unsubstituted or substituted with at least one of deuterium, a (C1-C30)alkyl(s), and a (C6-C30)aryl(s), a triphenylenyl unsubstituted or substituted with deuterium, a fluoranthenyl unsubstituted or substituted with deuterium, a phenanthrenyl unsubstituted or substituted with deuterium, a dibenzofuranyl unsubstituted or substituted with deuterium, a carbazolyl unsubstituted or substituted with deuterium, a dibenzothiophenyl unsubstituted or substituted with deuterium, or a combination thereof.

In formula 2, any one of X₁₅ to X₁₃ and any one of X₁₉ to X₂₂ are linked to each other to form a single bond; and X₁₁ to X₁₄, X₂₃ to X₂₆, and X₁₅ to X₂₂ which do not form a single bond each independently represent hydrogen, deuterium, a substituted or unsubstituted (C6-C30)aryl, or a substituted or unsubstituted (3- to 30-membered)heteroaryl; or may be linked to an adjacent substituent to form a ring(s). For example, any one of X₁₅ to X₁₈ and any one of X₁₉ to X₂₂ may be linked to each other to form a single bond; and X₁₁ to X₁₄, X₂₃ to X₂₆, and X₁₅ to X₂₂ which do not form a single bond may each independently be hydrogen, deuterium, etc.

According to another embodiment of the present disclosure, at least one, preferably at least two, more preferably at least three, and even more preferably all of of X₁₁, X₁₈, X₁₉, and X₂₆ may be deuterium.

According to one embodiment of the present disclosure, X₁₁ to X₂₆ may have a deuterium substitution rate of about 25% to about 100%, preferably about 35% to about 100%, more preferably about 45% to about 100%, and even more preferably about 55% to about 100%.

According to one embodiment of the present disclosure, formula 2 may be represented by at least one of the following formulas 2-1 to 2-8.

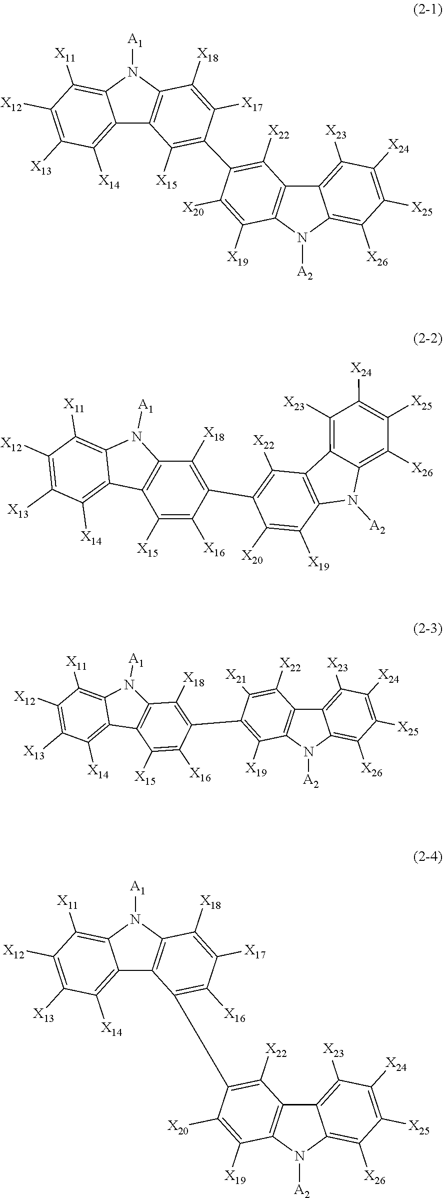

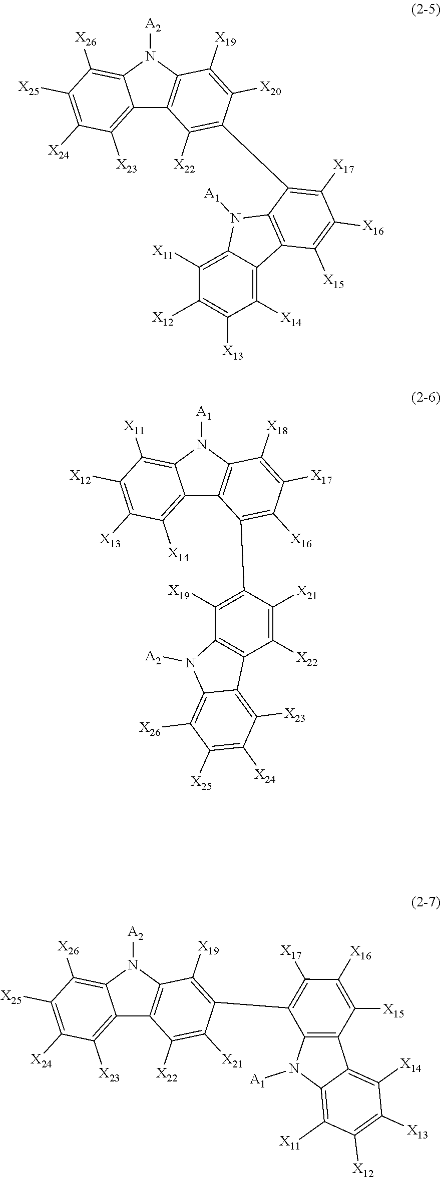

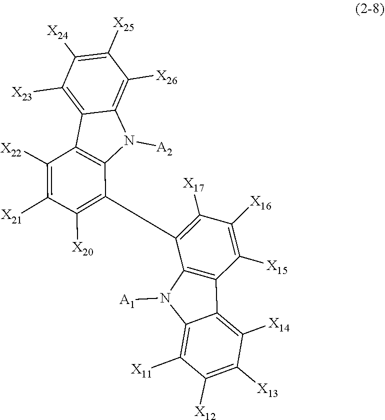

In formulas 2-1 to 2-8, A₁, A₂, and X₁₁ to X₂₆ are as defined in formula 2.

According to one embodiment of the present disclosure, when the compound of formula 2 contains deuterium, the deuterium substitution rate thereof may be about 40% to about 100%, preferably about 50% to about 100%, more preferably about 60% to about 100%, and even more preferably about 70% to about 100%. The compound of formula 2 having said deuterium substitution rate may increase the bond dissociation energy due to deuteration to increase the stability of the compound, and an organic electroluminescent device comprising the compound may exhibit improved lifespan properties.

According to one embodiment of the present disclosure, formula 1 may not contain deuterium, and formula 2 may contain deuterium.

The compound represented by formula 1 may be at least one selected from the following compounds, but is not limited thereto.

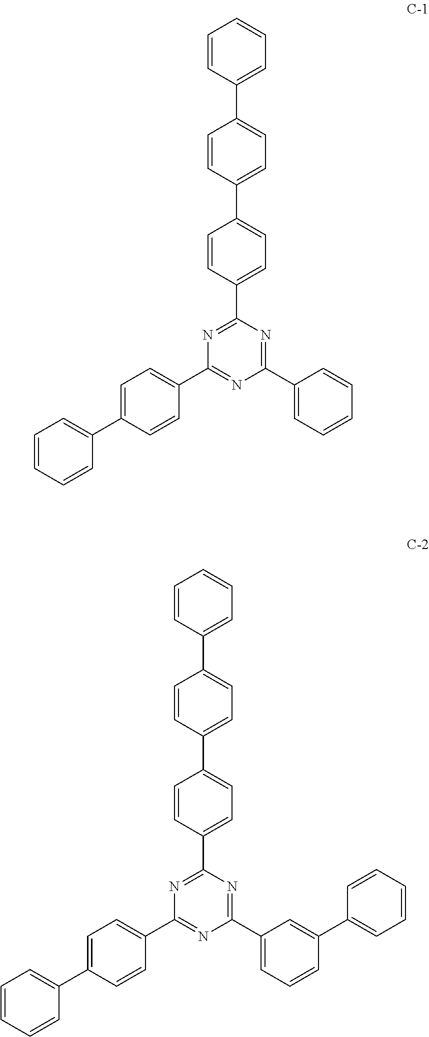 [SMILES: c1ccc(-c2ccc(-c3ccc(-c4nc(-c5ccccc5)nc(-c5ccc(-c6ccccc6)cc5)n4)cc3)cc2)cc1 ; c1ccc(-c2ccc(-c3ccc(-c4nc(-c5ccc(-c6ccccc6)cc5)nc(-c5cccc(-c6ccccc6)c5)n4)cc3)cc2)cc1]

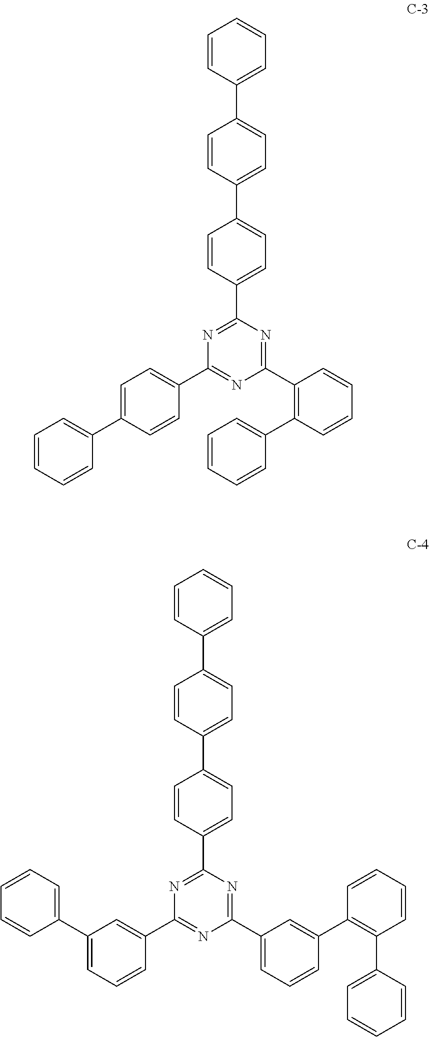 [SMILES: c1ccc(-c2ccc(-c3ccc(-c4nc(-c5ccc(-c6ccccc6)cc5)nc(-c5ccccc5-c5ccccc5)n4)cc3)cc2)cc1 ; c1ccc(-c2ccc(-c3ccc(-c4nc(-c5cccc(-c6ccccc6)c5)nc(-c5cccc(-c6ccccc6-c6ccccc6)c5)n4)cc3)cc2)cc1]

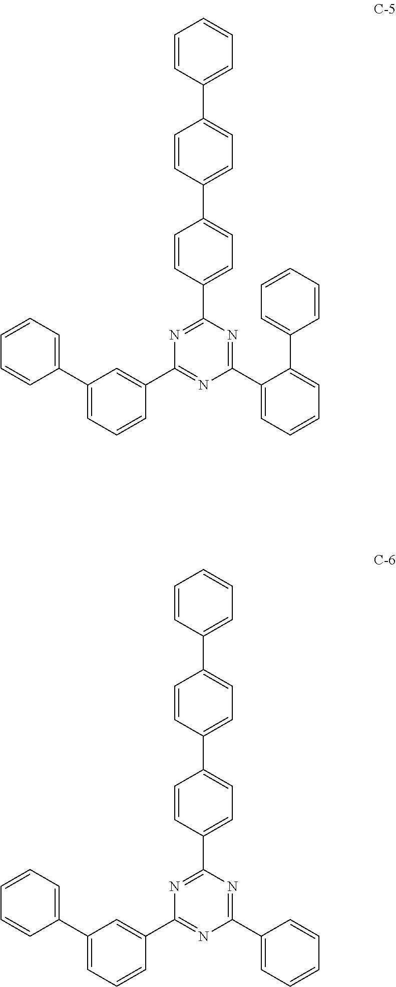 [SMILES: c1ccc(-c2ccc(-c3ccc(-c4nc(-c5cccc(-c6ccccc6)c5)nc(-c5ccccc5-c5ccccc5)n4)cc3)cc2)cc1 ; c1ccc(-c2ccc(-c3ccc(-c4nc(-c5ccccc5)nc(-c5cccc(-c6ccccc6)c5)n4)cc3)cc2)cc1]

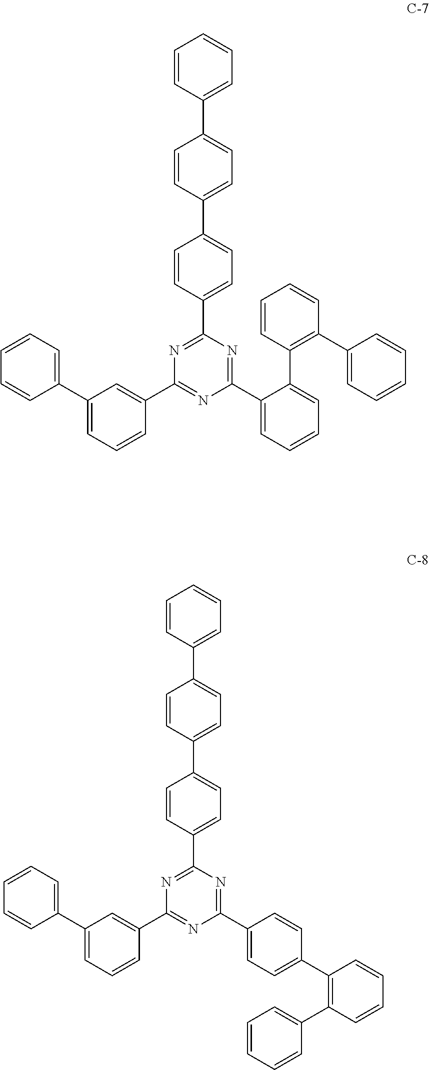 [SMILES: c1ccc(-c2ccc(-c3ccc(-c4nc(-c5cccc(-c6ccccc6)c5)nc(-c5ccccc5-c5ccccc5-c5ccccc5)n4)cc3)cc2)cc1 ; c1ccc(-c2ccc(-c3ccc(-c4nc(-c5ccc(-c6ccccc6-c6ccccc6)cc5)nc(-c5cccc(-c6ccccc6)c5)n4)cc3)cc2)cc1]

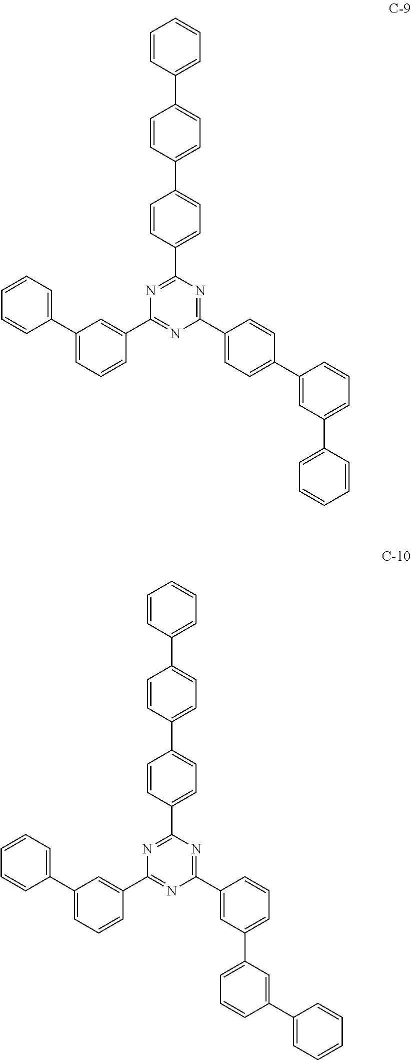 [SMILES: c1ccc(-c2ccc(-c3ccc(-c4nc(-c5ccc(-c6cccc(-c7ccccc7)c6)cc5)nc(-c5cccc(-c6ccccc6)c5)n4)cc3)cc2)cc1 ; c1ccc(-c2ccc(-c3ccc(-c4nc(-c5cccc(-c6ccccc6)c5)nc(-c5cccc(-c6cccc(-c7ccccc7)c6)c5)n4)cc3)cc2)cc1]

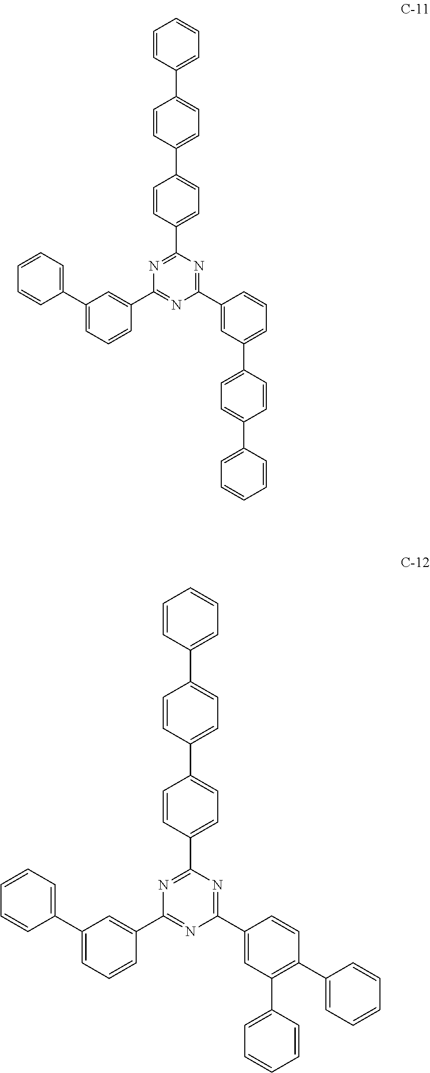 [SMILES: c1ccc(-c2ccc(-c3ccc(-c4nc(-c5cccc(-c6ccccc6)c5)nc(-c5cccc(-c6ccc(-c7ccccc7)cc6)c5)n4)cc3)cc2)cc1 ; c1ccc(-c2ccc(-c3ccc(-c4nc(-c5cccc(-c6ccccc6)c5)nc(-c5ccc(-c6ccccc6)c(-c6ccccc6)c5)n4)cc3)cc2)cc1]

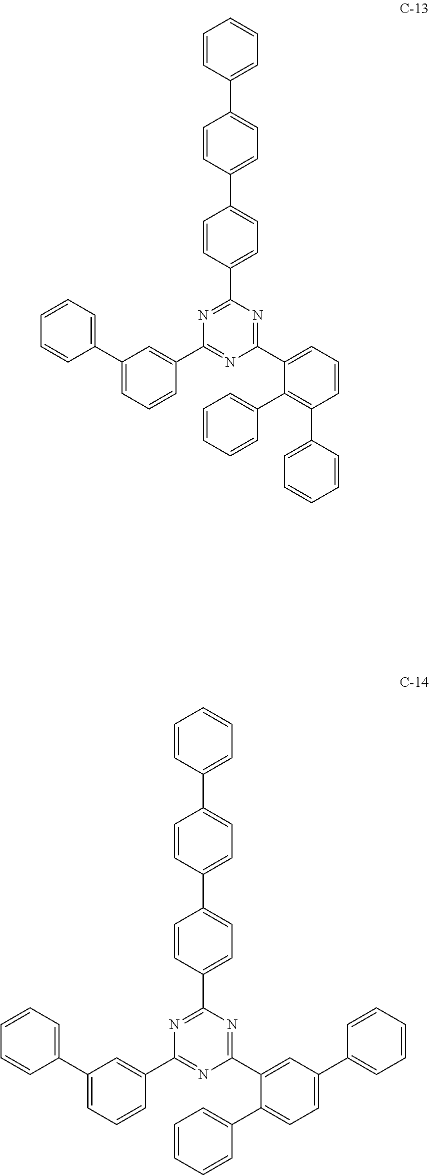 [SMILES: c1ccc(-c2ccc(-c3ccc(-c4nc(-c5cccc(-c6ccccc6)c5)nc(-c5cccc(-c6ccccc6)c5-c5ccccc5)n4)cc3)cc2)cc1 ; c1ccc(-c2ccc(-c3ccc(-c4nc(-c5cccc(-c6ccccc6)c5)nc(-c5cc(-c6ccccc6)ccc5-c5ccccc5)n4)cc3)cc2)cc1]

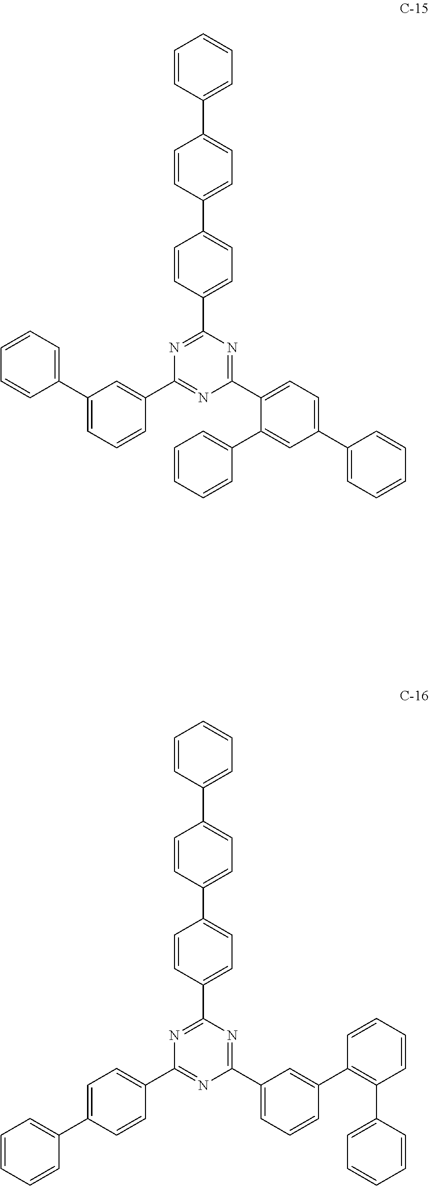 [SMILES: c1ccc(-c2ccc(-c3ccc(-c4nc(-c5cccc(-c6ccccc6)c5)nc(-c5ccc(-c6ccccc6)cc5-c5ccccc5)n4)cc3)cc2)cc1 ; c1ccc(-c2ccc(-c3ccc(-c4nc(-c5ccc(-c6ccccc6)cc5)nc(-c5cccc(-c6ccccc6-c6ccccc6)c5)n4)cc3)cc2)cc1]

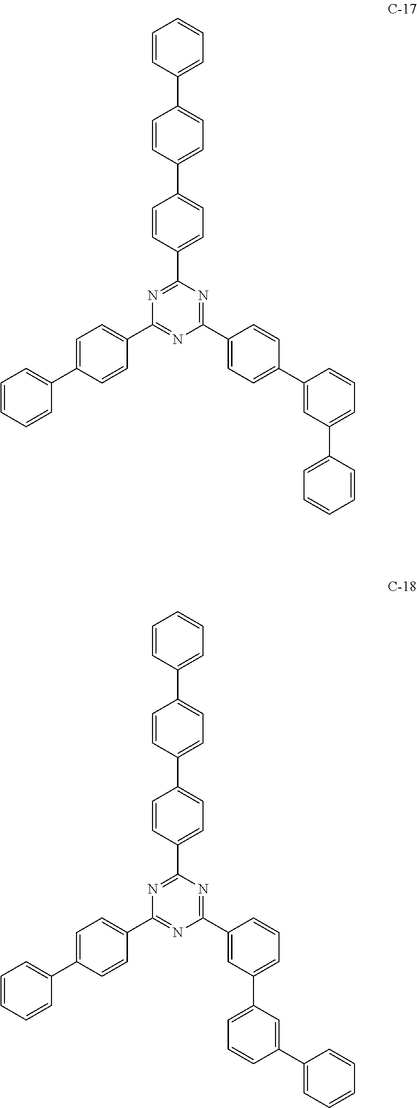 [SMILES: c1ccc(-c2ccc(-c3ccc(-c4nc(-c5ccc(-c6ccccc6)cc5)nc(-c5ccc(-c6cccc(-c7ccccc7)c6)cc5)n4)cc3)cc2)cc1 ; c1ccc(-c2ccc(-c3ccc(-c4nc(-c5ccc(-c6ccccc6)cc5)nc(-c5cccc(-c6cccc(-c7ccccc7)c6)c5)n4)cc3)cc2)cc1]

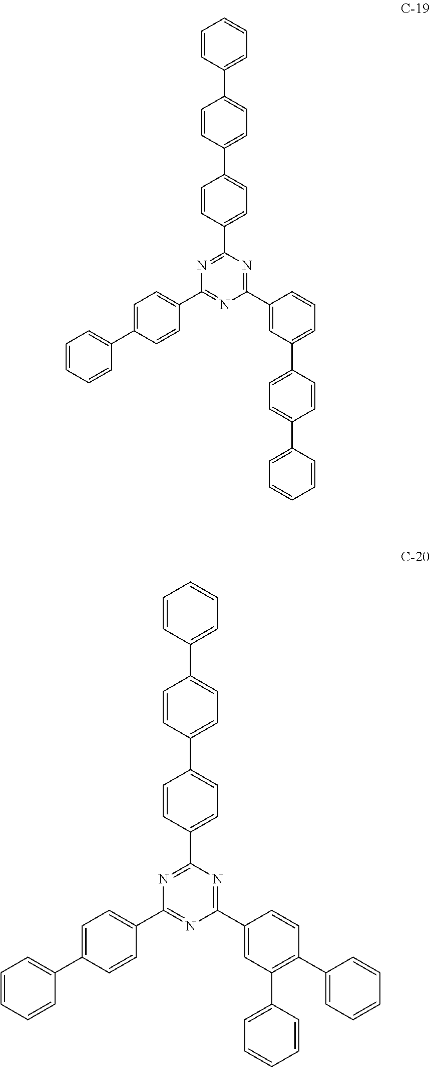 [SMILES: c1ccc(-c2ccc(-c3ccc(-c4nc(-c5ccc(-c6ccccc6)cc5)nc(-c5cccc(-c6ccc(-c7ccccc7)cc6)c5)n4)cc3)cc2)cc1 ; c1ccc(-c2ccc(-c3ccc(-c4nc(-c5ccc(-c6ccccc6)cc5)nc(-c5ccc(-c6ccccc6)c(-c6ccccc6)c5)n4)cc3)cc2)cc1]

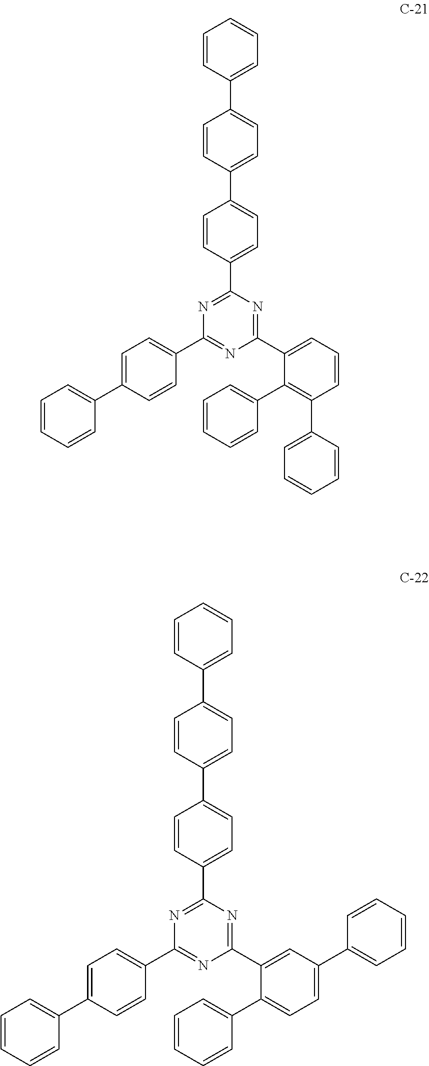 [SMILES: c1ccc(-c2ccc(-c3ccc(-c4nc(-c5ccc(-c6ccccc6)cc5)nc(-c5cccc(-c6ccccc6)c5-c5ccccc5)n4)cc3)cc2)cc1 ; c1ccc(-c2ccc(-c3ccc(-c4nc(-c5ccc(-c6ccccc6)cc5)nc(-c5cc(-c6ccccc6)ccc5-c5ccccc5)n4)cc3)cc2)cc1]

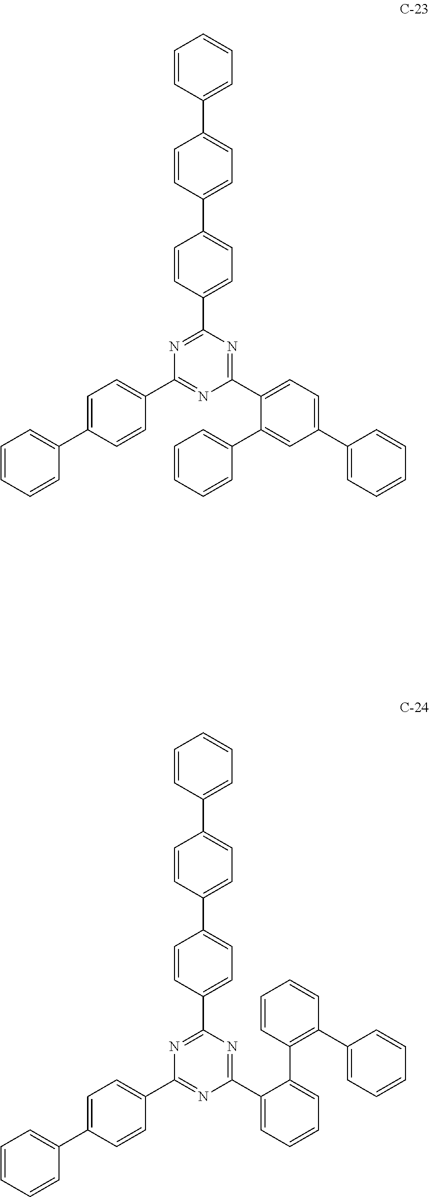 [SMILES: c1ccc(-c2ccc(-c3ccc(-c4nc(-c5ccc(-c6ccccc6)cc5)nc(-c5ccc(-c6ccccc6)cc5-c5ccccc5)n4)cc3)cc2)cc1 ; c1ccc(-c2ccc(-c3ccc(-c4nc(-c5ccc(-c6ccccc6)cc5)nc(-c5ccccc5-c5ccccc5-c5ccccc5)n4)cc3)cc2)cc1]

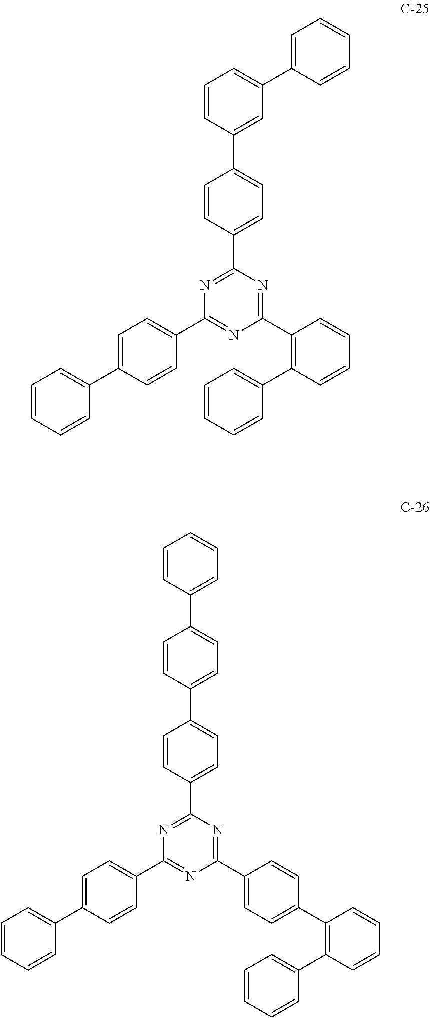 [SMILES: c1ccc(-c2ccc(-c3nc(-c4ccc(-c5cccc(-c6ccccc6)c5)cc4)nc(-c4ccccc4-c4ccccc4)n3)cc2)cc1 ; c1ccc(-c2ccc(-c3ccc(-c4nc(-c5ccc(-c6ccccc6)cc5)nc(-c5ccc(-c6ccccc6-c6ccccc6)cc5)n4)cc3)cc2)cc1]

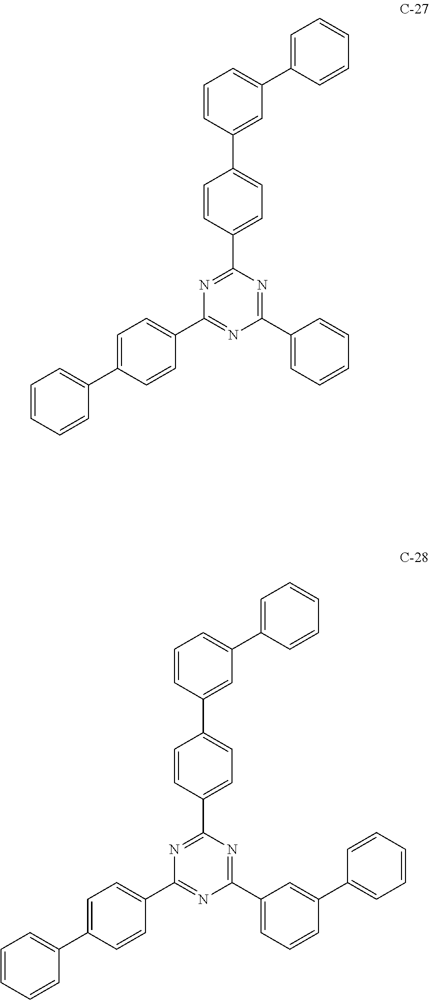 [SMILES: c1ccc(-c2ccc(-c3nc(-c4ccccc4)nc(-c4ccc(-c5cccc(-c6ccccc6)c5)cc4)n3)cc2)cc1 ; c1ccc(-c2ccc(-c3nc(-c4ccc(-c5cccc(-c6ccccc6)c5)cc4)nc(-c4cccc(-c5ccccc5)c4)n3)cc2)cc1]

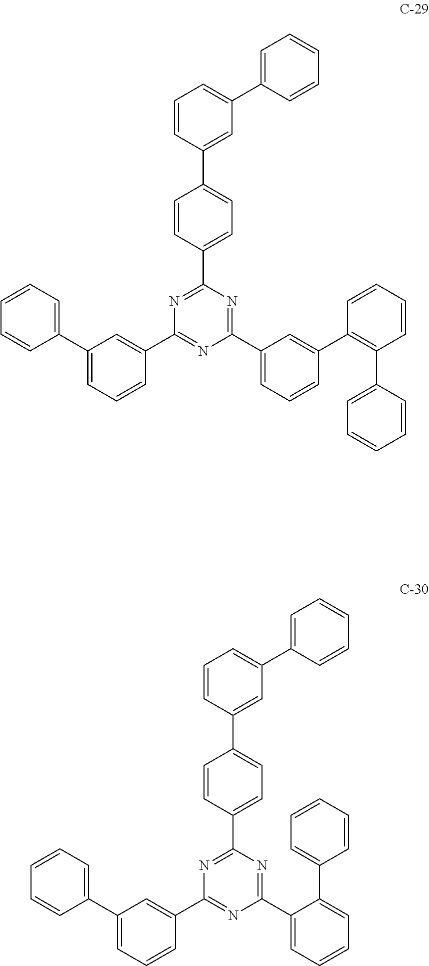 [SMILES: c1ccc(-c2cccc(-c3ccc(-c4nc(-c5cccc(-c6ccccc6)c5)nc(-c5cccc(-c6ccccc6-c6ccccc6)c5)n4)cc3)c2)cc1 ; c1ccc(-c2cccc(-c3ccc(-c4nc(-c5cccc(-c6ccccc6)c5)nc(-c5ccccc5-c5ccccc5)n4)cc3)c2)cc1]

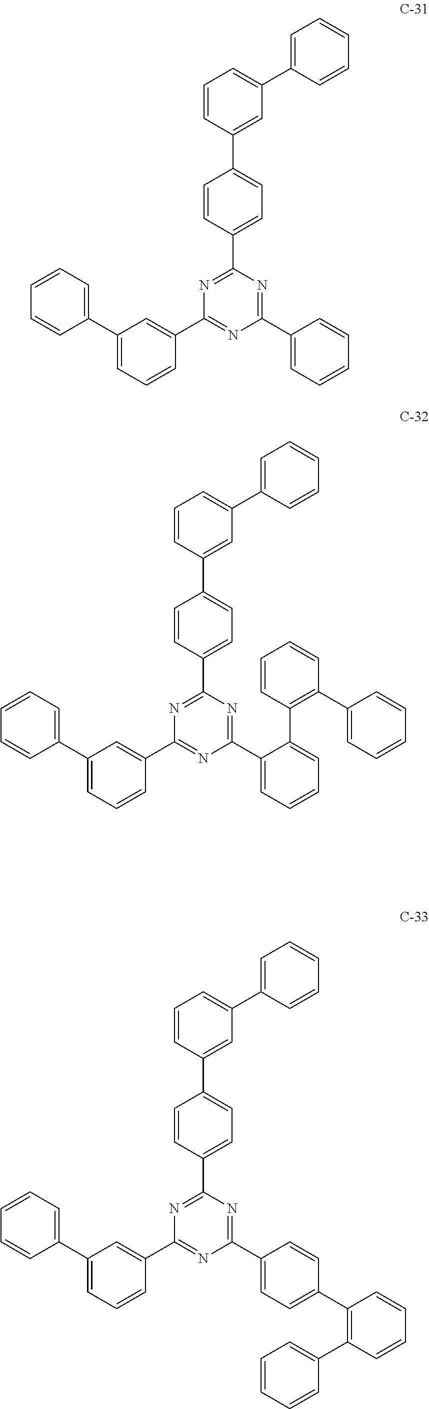 [SMILES: c1ccc(-c2cccc(-c3ccc(-c4nc(-c5ccccc5)nc(-c5cccc(-c6ccccc6)c5)n4)cc3)c2)cc1 ; c1ccc(-c2cccc(-c3ccc(-c4nc(-c5cccc(-c6ccccc6)c5)nc(-c5ccccc5-c5ccccc5-c5ccccc5)n4)cc3)c2)cc1 ; c1ccc(-c2cccc(-c3ccc(-c4nc(-c5ccc(-c6ccccc6-c6ccccc6)cc5)nc(-c5cccc(-c6ccccc6)c5)n4)cc3)c2)cc1]

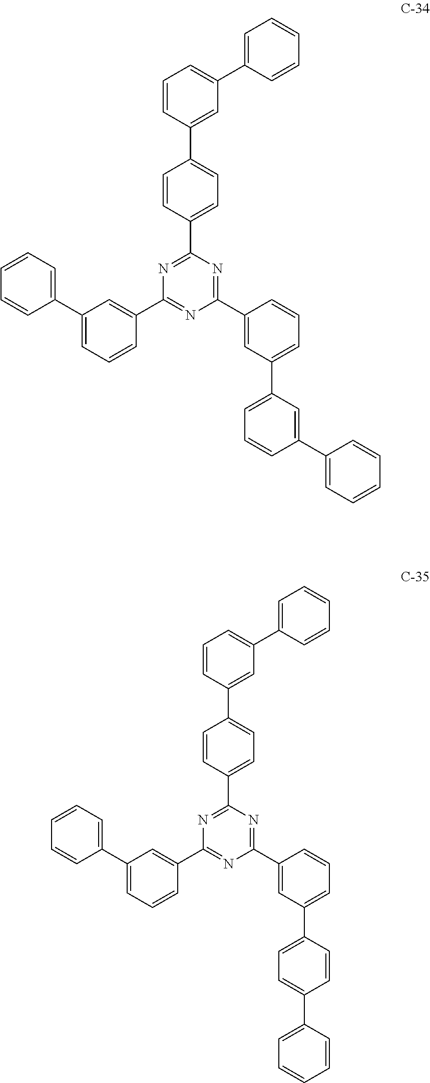 [SMILES: c1ccc(-c2cccc(-c3ccc(-c4nc(-c5cccc(-c6ccccc6)c5)nc(-c5cccc(-c6cccc(-c7ccccc7)c6)c5)n4)cc3)c2)cc1 ; c1ccc(-c2ccc(-c3cccc(-c4nc(-c5ccc(-c6cccc(-c7ccccc7)c6)cc5)nc(-c5cccc(-c6ccccc6)c5)n4)c3)cc2)cc1]

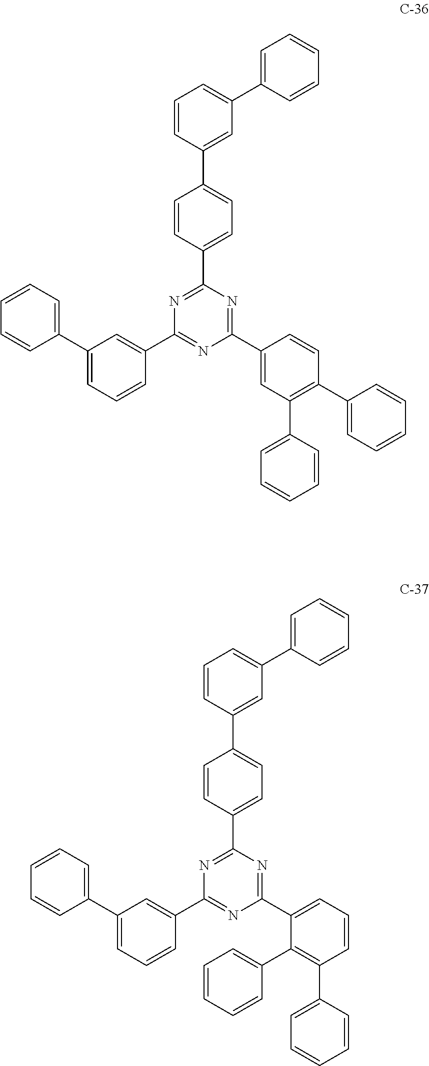 [SMILES: c1ccc(-c2cccc(-c3ccc(-c4nc(-c5cccc(-c6ccccc6)c5)nc(-c5ccc(-c6ccccc6)c(-c6ccccc6)c5)n4)cc3)c2)cc1 ; c1ccc(-c2cccc(-c3ccc(-c4nc(-c5cccc(-c6ccccc6)c5)nc(-c5cccc(-c6ccccc6)c5-c5ccccc5)n4)cc3)c2)cc1]

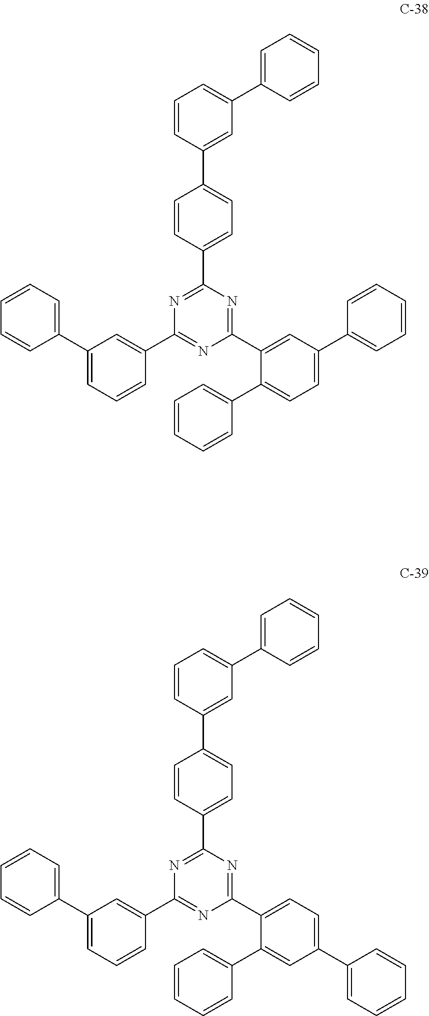 [SMILES: c1ccc(-c2cccc(-c3ccc(-c4nc(-c5cccc(-c6ccccc6)c5)nc(-c5cc(-c6ccccc6)ccc5-c5ccccc5)n4)cc3)c2)cc1 ; c1ccc(-c2cccc(-c3ccc(-c4nc(-c5cccc(-c6ccccc6)c5)nc(-c5ccc(-c6ccccc6)cc5-c5ccccc5)n4)cc3)c2)cc1]

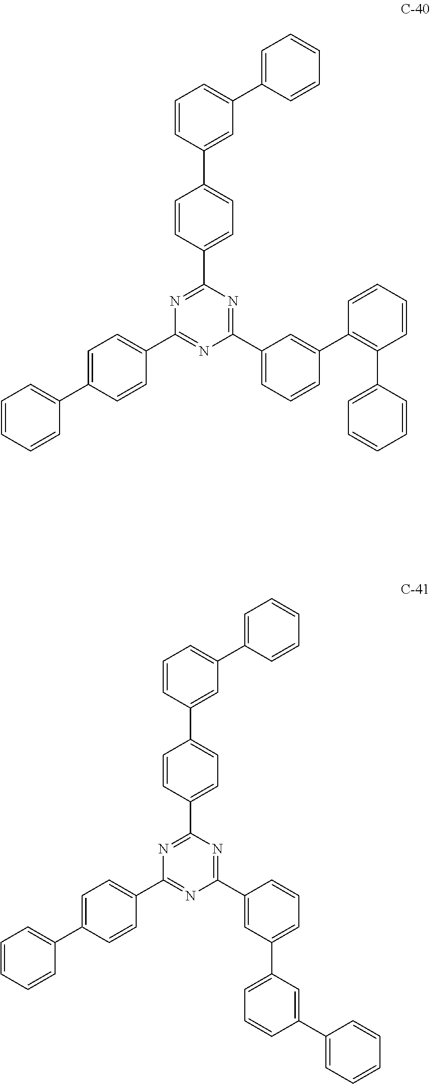 [SMILES: c1ccc(-c2ccc(-c3nc(-c4ccc(-c5cccc(-c6ccccc6)c5)cc4)nc(-c4cccc(-c5ccccc5-c5ccccc5)c4)n3)cc2)cc1 ; c1ccc(-c2ccc(-c3nc(-c4ccc(-c5cccc(-c6ccccc6)c5)cc4)nc(-c4cccc(-c5cccc(-c6ccccc6)c5)c4)n3)cc2)cc1]

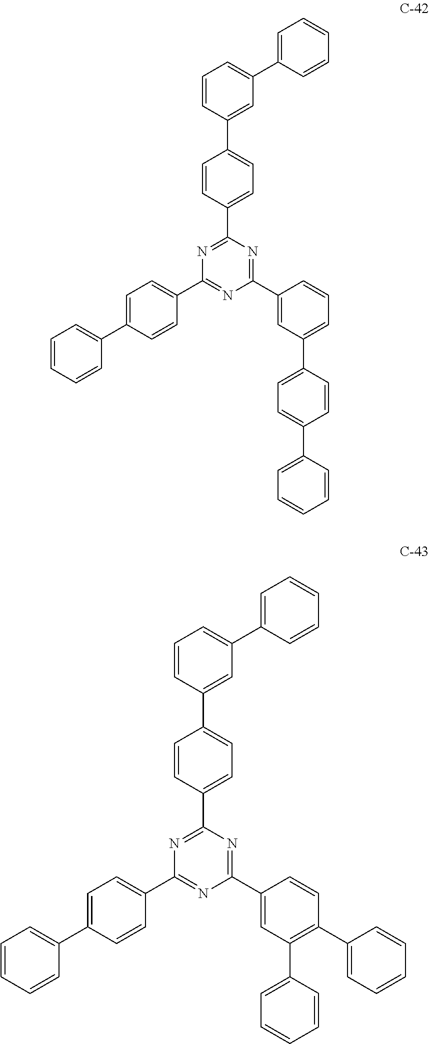 [SMILES: c1ccc(-c2ccc(-c3cccc(-c4nc(-c5ccc(-c6ccccc6)cc5)nc(-c5ccc(-c6cccc(-c7ccccc7)c6)cc5)n4)c3)cc2)cc1 ; c1ccc(-c2ccc(-c3nc(-c4ccc(-c5cccc(-c6ccccc6)c5)cc4)nc(-c4ccc(-c5ccccc5)c(-c5ccccc5)c4)n3)cc2)cc1]

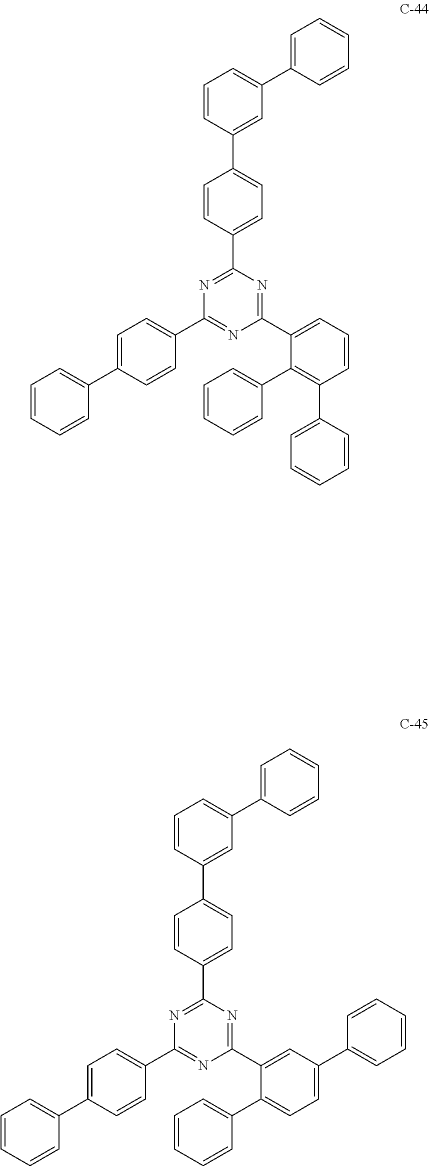 [SMILES: c1ccc(-c2ccc(-c3nc(-c4ccc(-c5cccc(-c6ccccc6)c5)cc4)nc(-c4cccc(-c5ccccc5)c4-c4ccccc4)n3)cc2)cc1 ; c1ccc(-c2ccc(-c3nc(-c4ccc(-c5cccc(-c6ccccc6)c5)cc4)nc(-c4cc(-c5ccccc5)ccc4-c4ccccc4)n3)cc2)cc1]

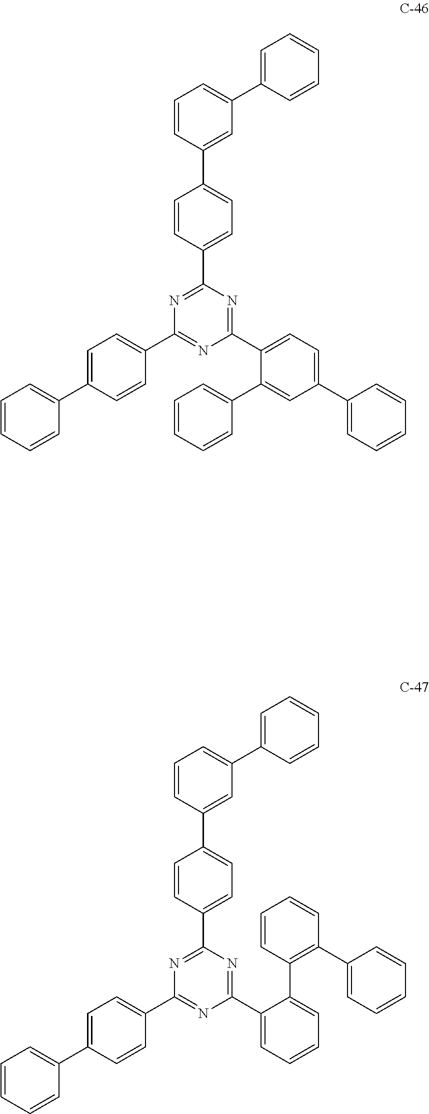 [SMILES: c1ccc(-c2ccc(-c3nc(-c4ccc(-c5cccc(-c6ccccc6)c5)cc4)nc(-c4ccc(-c5ccccc5)cc4-c4ccccc4)n3)cc2)cc1 ; c1ccc(-c2ccc(-c3nc(-c4ccc(-c5cccc(-c6ccccc6)c5)cc4)nc(-c4ccccc4-c4ccccc4-c4ccccc4)n3)cc2)cc1]

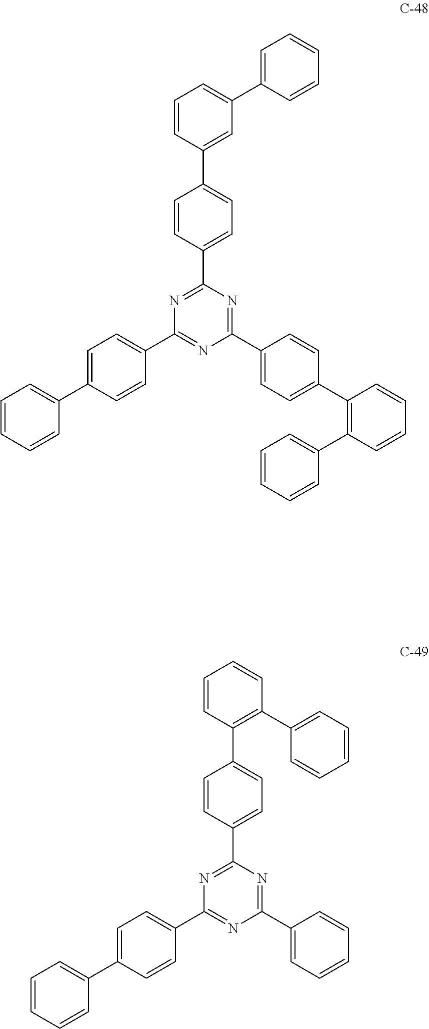 [SMILES: c1ccc(-c2ccc(-c3nc(-c4ccc(-c5cccc(-c6ccccc6)c5)cc4)nc(-c4ccc(-c5ccccc5-c5ccccc5)cc4)n3)cc2)cc1 ; c1ccc(-c2ccc(-c3nc(-c4ccccc4)nc(-c4ccc(-c5ccccc5-c5ccccc5)cc4)n3)cc2)cc1]

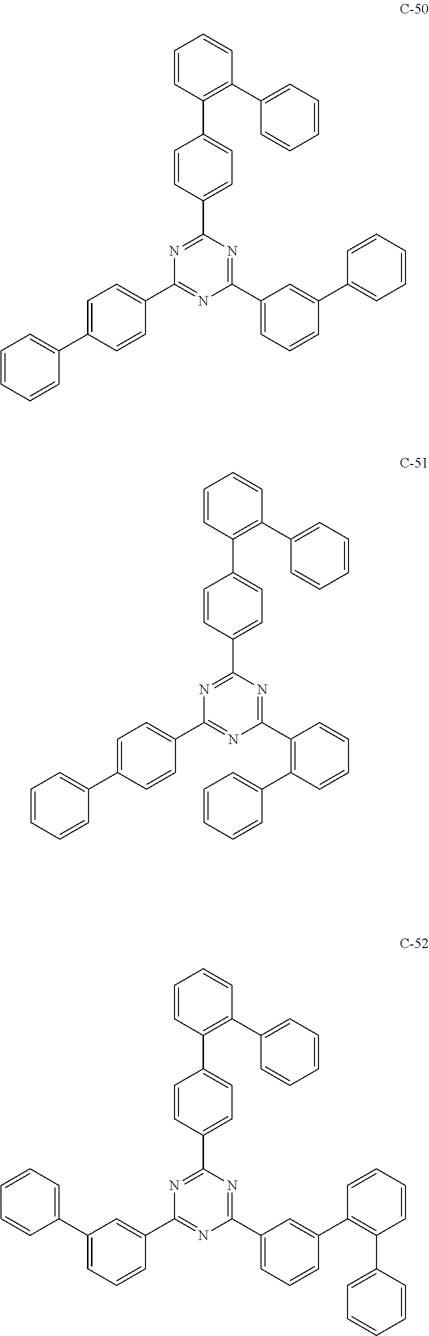 [SMILES: c1ccc(-c2ccc(-c3nc(-c4ccc(-c5ccccc5-c5ccccc5)cc4)nc(-c4cccc(-c5ccccc5)c4)n3)cc2)cc1 ; c1ccc(-c2ccc(-c3nc(-c4ccc(-c5ccccc5-c5ccccc5)cc4)nc(-c4ccccc4-c4ccccc4)n3)cc2)cc1 ; c1ccc(-c2cccc(-c3nc(-c4ccc(-c5ccccc5-c5ccccc5)cc4)nc(-c4cccc(-c5ccccc5-c5ccccc5)c4)n3)c2)cc1]

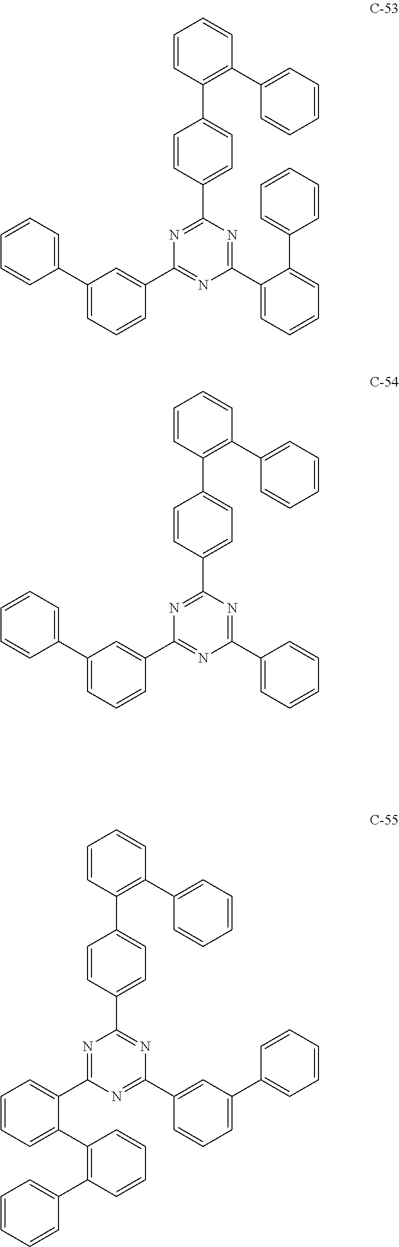 [SMILES: c1ccc(-c2cccc(-c3nc(-c4ccc(-c5ccccc5-c5ccccc5)cc4)nc(-c4ccccc4-c4ccccc4)n3)c2)cc1 ; c1ccc(-c2cccc(-c3nc(-c4ccccc4)nc(-c4ccc(-c5ccccc5-c5ccccc5)cc4)n3)c2)cc1 ; c1ccc(-c2cccc(-c3nc(-c4ccc(-c5ccccc5-c5ccccc5)cc4)nc(-c4ccccc4-c4ccccc4-c4ccccc4)n3)c2)cc1]

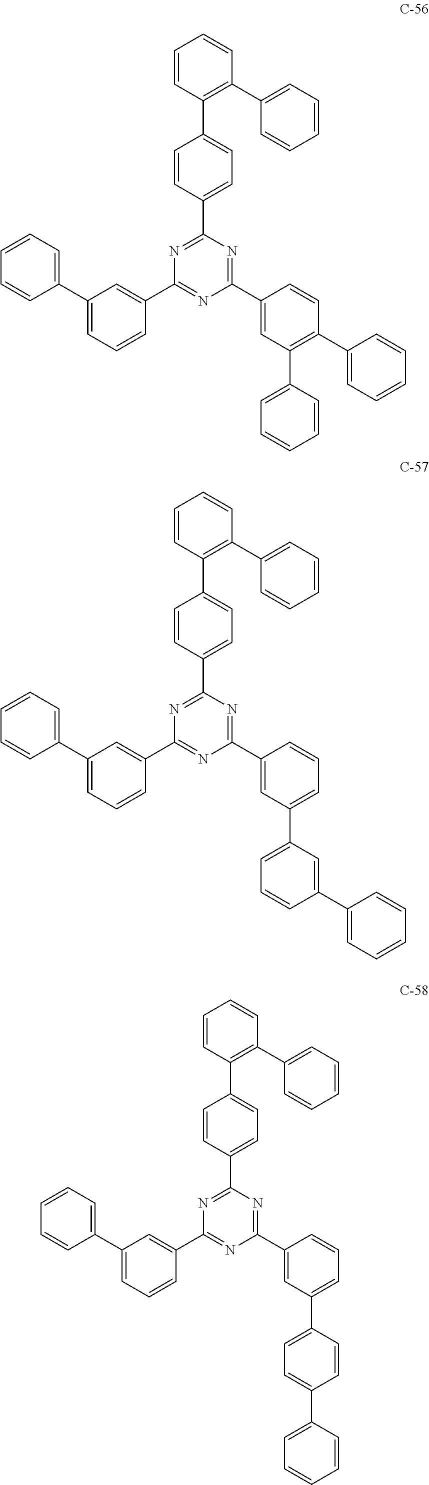 [SMILES: c1ccc(-c2cccc(-c3nc(-c4ccc(-c5ccccc5-c5ccccc5)cc4)nc(-c4ccc(-c5ccccc5)c(-c5ccccc5)c4)n3)c2)cc1 ; c1ccc(-c2cccc(-c3cccc(-c4nc(-c5ccc(-c6ccccc6-c6ccccc6)cc5)nc(-c5cccc(-c6ccccc6)c5)n4)c3)c2)cc1 ; c1ccc(-c2ccc(-c3cccc(-c4nc(-c5ccc(-c6ccccc6-c6ccccc6)cc5)nc(-c5cccc(-c6ccccc6)c5)n4)c3)cc2)cc1]

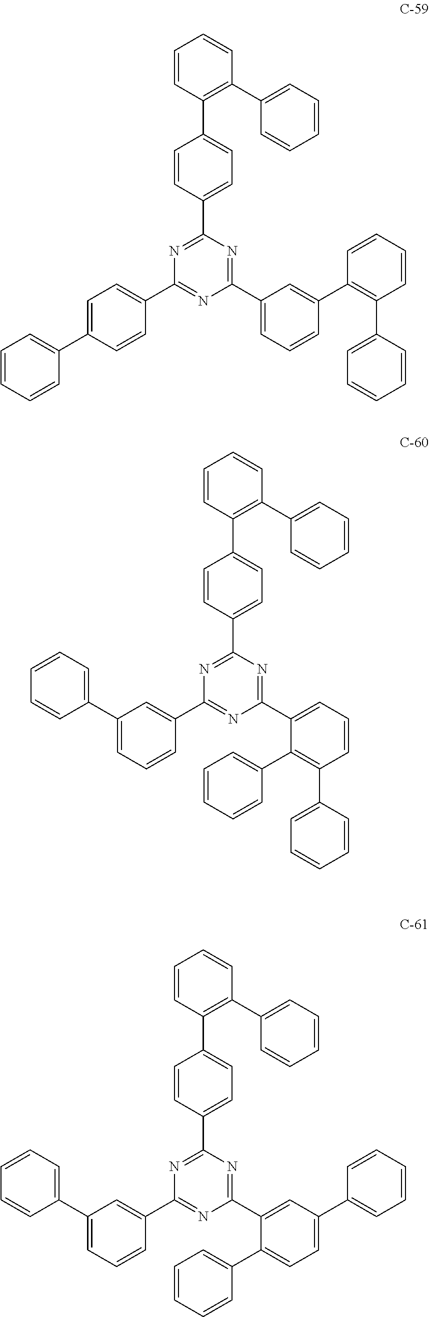 [SMILES: c1ccc(-c2ccc(-c3nc(-c4ccc(-c5ccccc5-c5ccccc5)cc4)nc(-c4cccc(-c5ccccc5-c5ccccc5)c4)n3)cc2)cc1 ; c1ccc(-c2cccc(-c3nc(-c4ccc(-c5ccccc5-c5ccccc5)cc4)nc(-c4cccc(-c5ccccc5)c4-c4ccccc4)n3)c2)cc1 ; c1ccc(-c2cccc(-c3nc(-c4ccc(-c5ccccc5-c5ccccc5)cc4)nc(-c4cc(-c5ccccc5)ccc4-c4ccccc4)n3)c2)cc1]

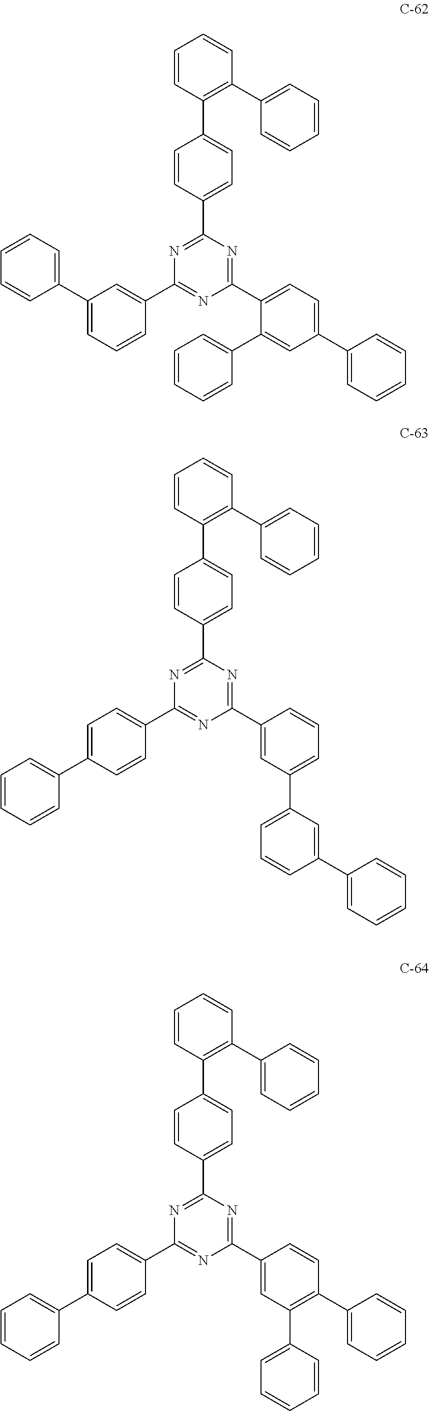 [SMILES: c1ccc(-c2cccc(-c3nc(-c4ccc(-c5ccccc5-c5ccccc5)cc4)nc(-c4ccc(-c5ccccc5)cc4-c4ccccc4)n3)c2)cc1 ; c1ccc(-c2ccc(-c3nc(-c4ccc(-c5ccccc5-c5ccccc5)cc4)nc(-c4cccc(-c5cccc(-c6ccccc6)c5)c4)n3)cc2)cc1 ; c1ccc(-c2ccc(-c3nc(-c4ccc(-c5ccccc5-c5ccccc5)cc4)nc(-c4ccc(-c5ccccc5)c(-c5ccccc5)c4)n3)cc2)cc1]

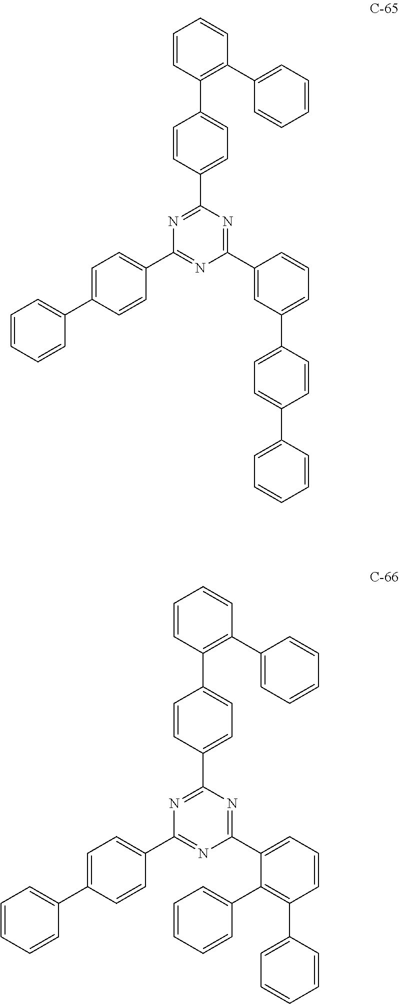 [SMILES: c1ccc(-c2ccc(-c3cccc(-c4nc(-c5ccc(-c6ccccc6)cc5)nc(-c5ccc(-c6ccccc6-c6ccccc6)cc5)n4)c3)cc2)cc1 ; c1ccc(-c2ccc(-c3nc(-c4ccc(-c5ccccc5-c5ccccc5)cc4)nc(-c4cccc(-c5ccccc5)c4-c4ccccc4)n3)cc2)cc1]

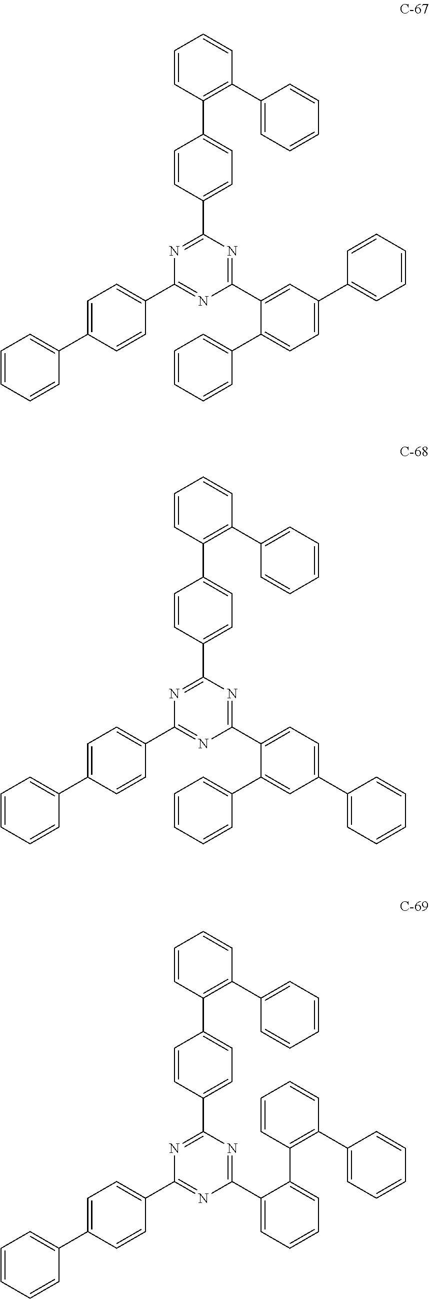 [SMILES: c1ccc(-c2ccc(-c3nc(-c4ccc(-c5ccccc5-c5ccccc5)cc4)nc(-c4cc(-c5ccccc5)ccc4-c4ccccc4)n3)cc2)cc1 ; c1ccc(-c2ccc(-c3nc(-c4ccc(-c5ccccc5-c5ccccc5)cc4)nc(-c4ccc(-c5ccccc5)cc4-c4ccccc4)n3)cc2)cc1 ; c1ccc(-c2ccc(-c3nc(-c4ccc(-c5ccccc5-c5ccccc5)cc4)nc(-c4ccccc4-c4ccccc4-c4ccccc4)n3)cc2)cc1]

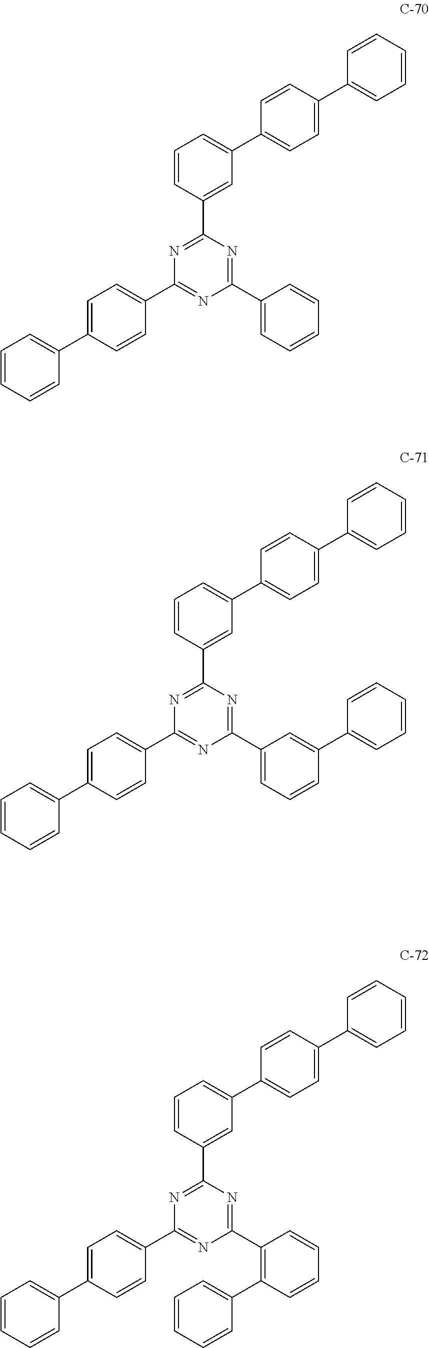 [SMILES: c1ccc(-c2ccc(-c3cccc(-c4nc(-c5ccccc5)nc(-c5ccc(-c6ccccc6)cc5)n4)c3)cc2)cc1 ; c1ccc(-c2ccc(-c3cccc(-c4nc(-c5ccc(-c6ccccc6)cc5)nc(-c5cccc(-c6ccccc6)c5)n4)c3)cc2)cc1 ; c1ccc(-c2ccc(-c3cccc(-c4nc(-c5ccc(-c6ccccc6)cc5)nc(-c5ccccc5-c5ccccc5)n4)c3)cc2)cc1]

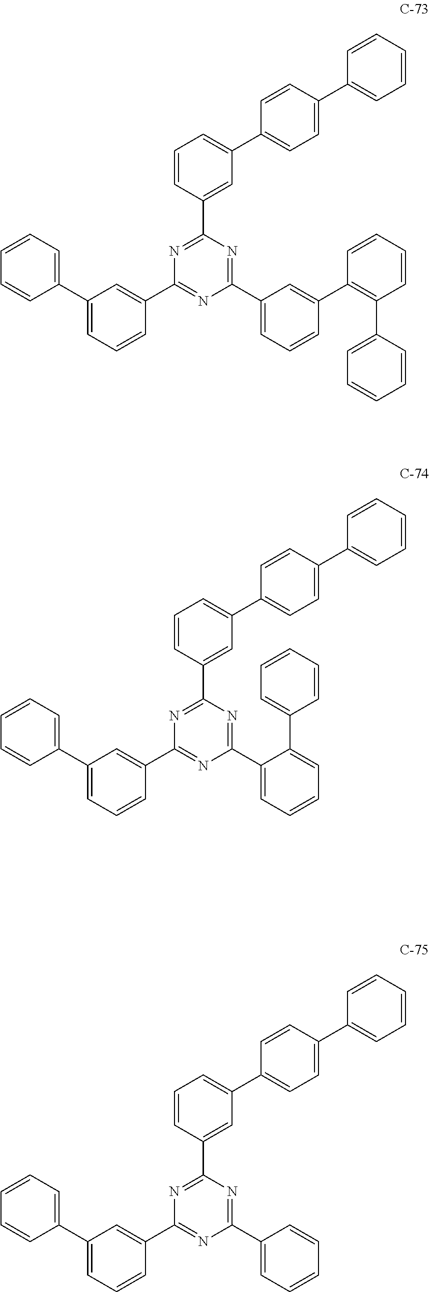 [SMILES: c1ccc(-c2ccc(-c3cccc(-c4nc(-c5cccc(-c6ccccc6)c5)nc(-c5cccc(-c6ccccc6-c6ccccc6)c5)n4)c3)cc2)cc1 ; c1ccc(-c2ccc(-c3cccc(-c4nc(-c5cccc(-c6ccccc6)c5)nc(-c5ccccc5-c5ccccc5)n4)c3)cc2)cc1 ; c1ccc(-c2ccc(-c3cccc(-c4nc(-c5ccccc5)nc(-c5cccc(-c6ccccc6)c5)n4)c3)cc2)cc1]

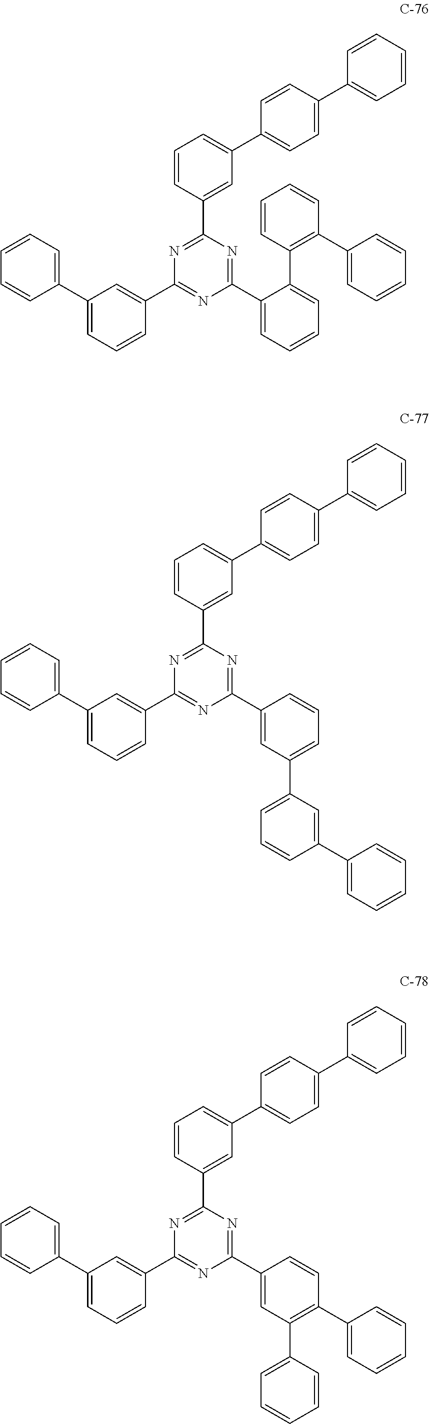 [SMILES: c1ccc(-c2ccc(-c3cccc(-c4nc(-c5cccc(-c6ccccc6)c5)nc(-c5ccccc5-c5ccccc5-c5ccccc5)n4)c3)cc2)cc1 ; c1ccc(-c2ccc(-c3cccc(-c4nc(-c5cccc(-c6ccccc6)c5)nc(-c5cccc(-c6cccc(-c7ccccc7)c6)c5)n4)c3)cc2)cc1 ; c1ccc(-c2ccc(-c3cccc(-c4nc(-c5cccc(-c6ccccc6)c5)nc(-c5ccc(-c6ccccc6)c(-c6ccccc6)c5)n4)c3)cc2)cc1]

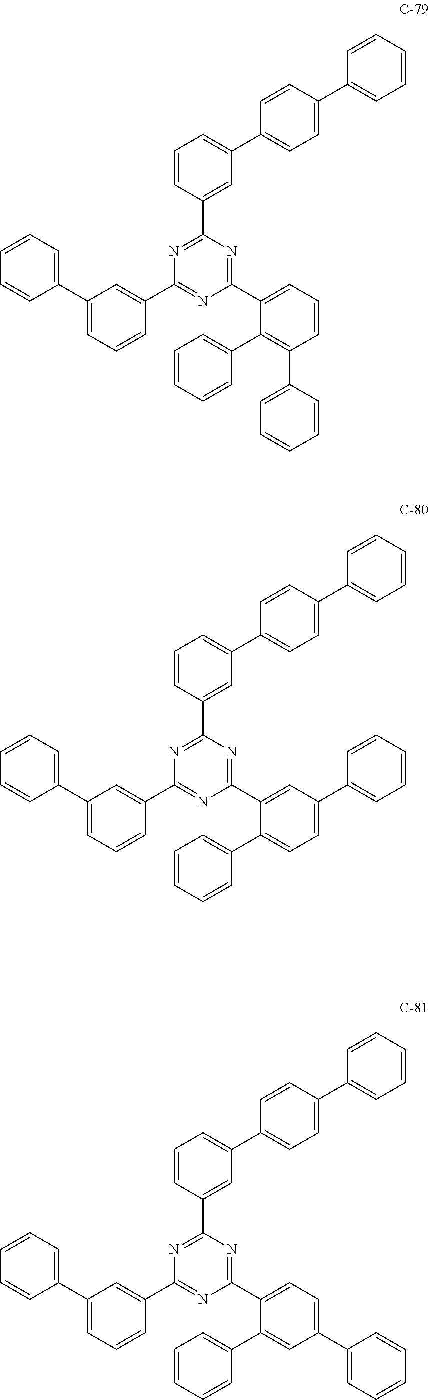 [SMILES: c1ccc(-c2ccc(-c3cccc(-c4nc(-c5cccc(-c6ccccc6)c5)nc(-c5cccc(-c6ccccc6)c5-c5ccccc5)n4)c3)cc2)cc1 ; c1ccc(-c2ccc(-c3cccc(-c4nc(-c5cccc(-c6ccccc6)c5)nc(-c5cc(-c6ccccc6)ccc5-c5ccccc5)n4)c3)cc2)cc1 ; c1ccc(-c2ccc(-c3cccc(-c4nc(-c5cccc(-c6ccccc6)c5)nc(-c5ccc(-c6ccccc6)cc5-c5ccccc5)n4)c3)cc2)cc1]

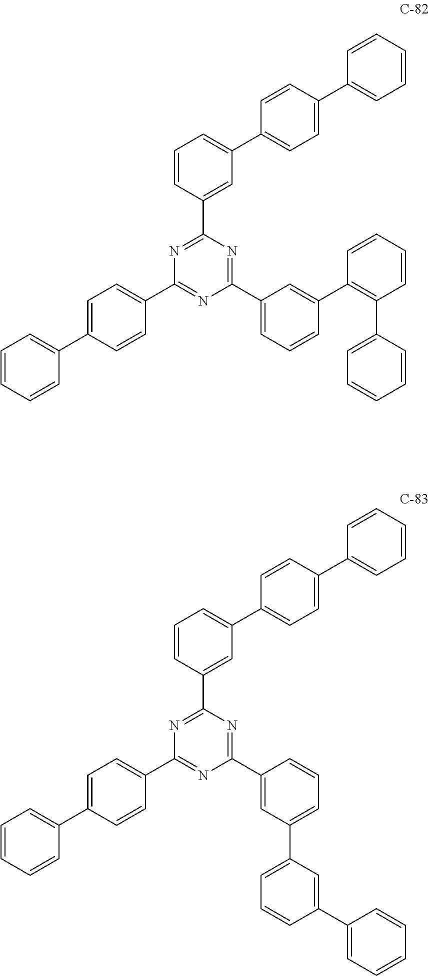 [SMILES: c1ccc(-c2ccc(-c3cccc(-c4nc(-c5ccc(-c6ccccc6)cc5)nc(-c5cccc(-c6ccccc6-c6ccccc6)c5)n4)c3)cc2)cc1 ; c1ccc(-c2ccc(-c3cccc(-c4nc(-c5ccc(-c6ccccc6)cc5)nc(-c5cccc(-c6cccc(-c7ccccc7)c6)c5)n4)c3)cc2)cc1]

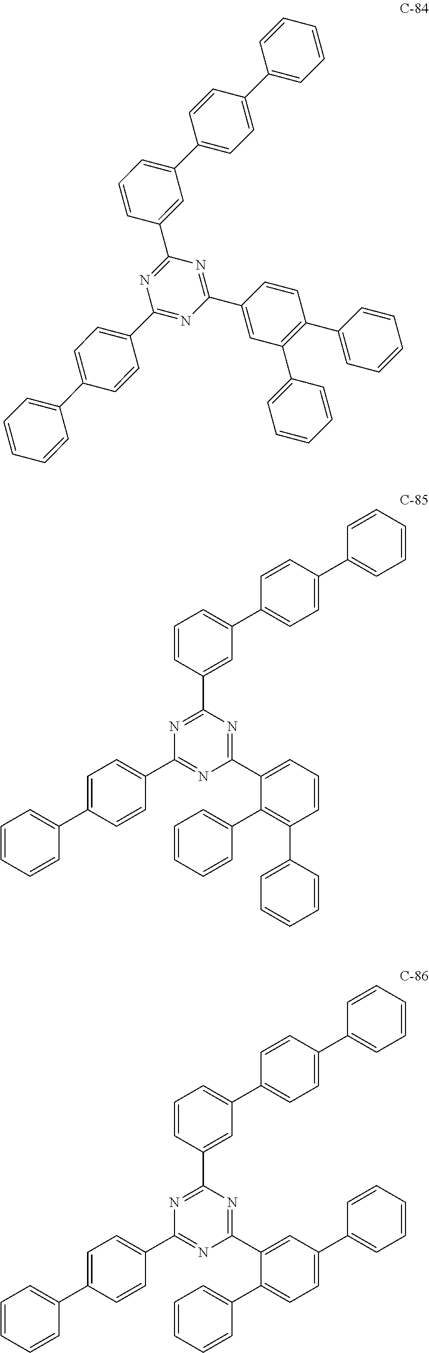 [SMILES: c1ccc(-c2ccc(-c3cccc(-c4nc(-c5ccc(-c6ccccc6)cc5)nc(-c5ccc(-c6ccccc6)c(-c6ccccc6)c5)n4)c3)cc2)cc1 ; c1ccc(-c2ccc(-c3cccc(-c4nc(-c5ccc(-c6ccccc6)cc5)nc(-c5cccc(-c6ccccc6)c5-c5ccccc5)n4)c3)cc2)cc1 ; c1ccc(-c2ccc(-c3cccc(-c4nc(-c5ccc(-c6ccccc6)cc5)nc(-c5cc(-c6ccccc6)ccc5-c5ccccc5)n4)c3)cc2)cc1]

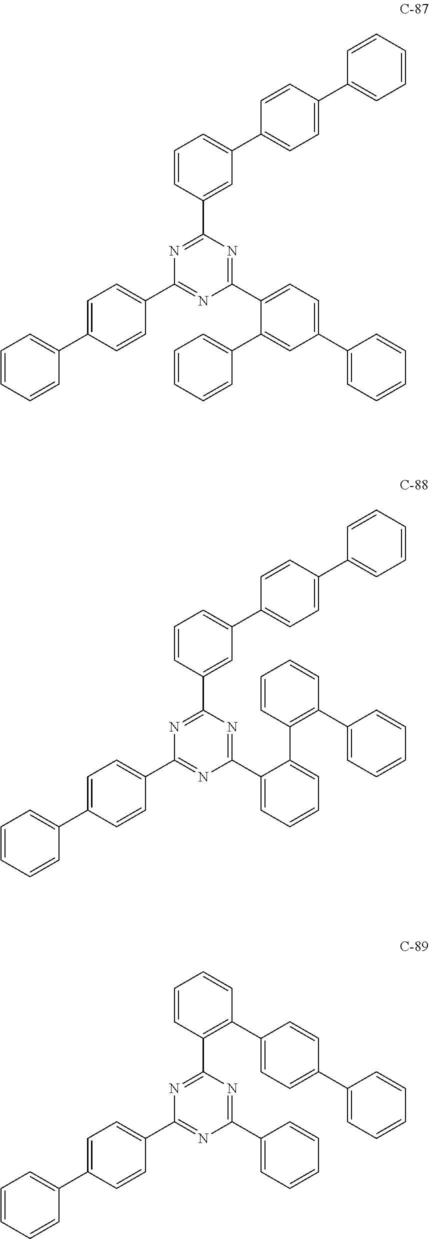 [SMILES: c1ccc(-c2ccc(-c3cccc(-c4nc(-c5ccc(-c6ccccc6)cc5)nc(-c5ccc(-c6ccccc6)cc5-c5ccccc5)n4)c3)cc2)cc1 ; c1ccc(-c2ccc(-c3cccc(-c4nc(-c5ccc(-c6ccccc6)cc5)nc(-c5ccccc5-c5ccccc5-c5ccccc5)n4)c3)cc2)cc1 ; c1ccc(-c2ccc(-c3nc(-c4ccccc4)nc(-c4ccccc4-c4ccc(-c5ccccc5)cc4)n3)cc2)cc1]

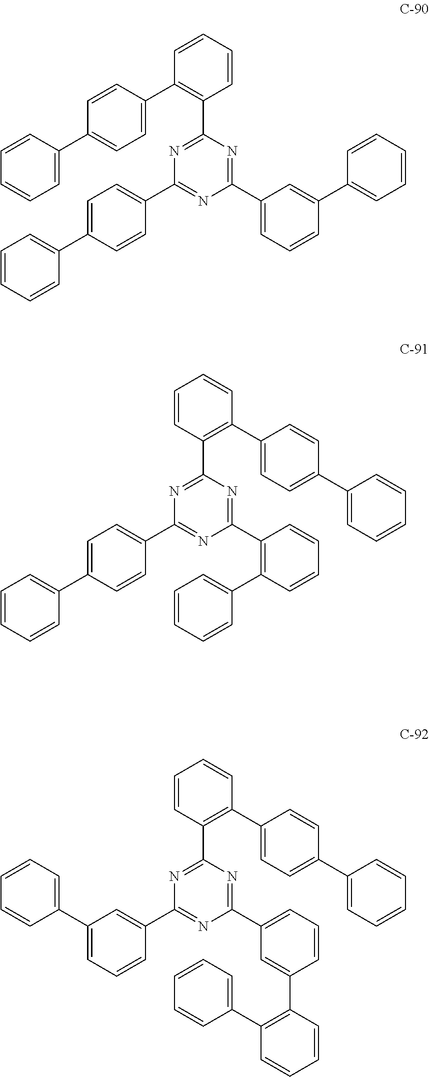 [SMILES: c1ccc(-c2ccc(-c3nc(-c4cccc(-c5ccccc5)c4)nc(-c4ccccc4-c4ccc(-c5ccccc5)cc4)n3)cc2)cc1 ; c1ccc(-c2ccc(-c3nc(-c4ccccc4-c4ccccc4)nc(-c4ccccc4-c4ccc(-c5ccccc5)cc4)n3)cc2)cc1 ; c1ccc(-c2ccc(-c3ccccc3-c3nc(-c4cccc(-c5ccccc5)c4)nc(-c4cccc(-c5ccccc5-c5ccccc5)c4)n3)cc2)cc1]

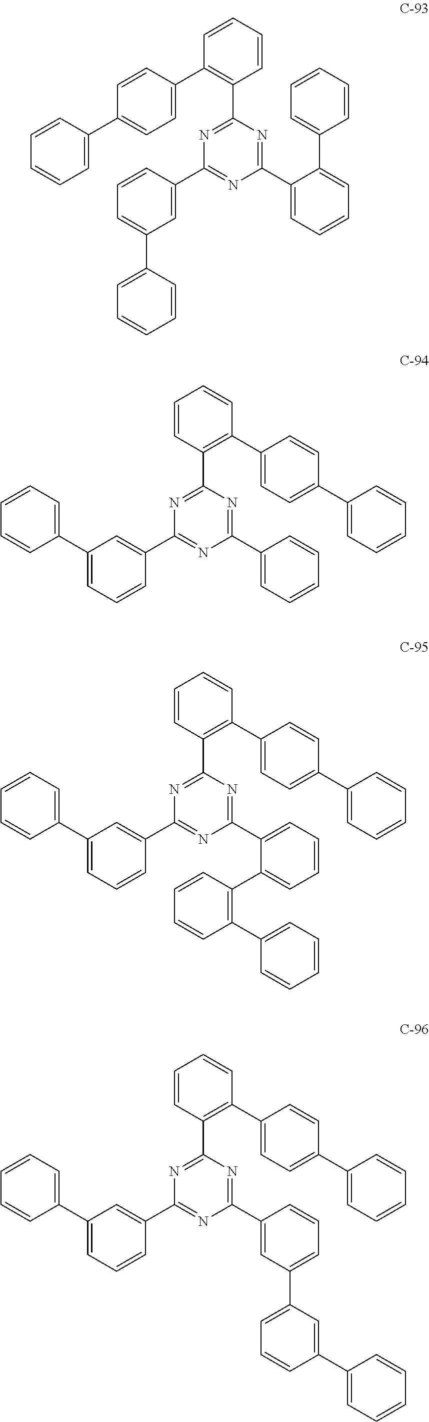 [SMILES: c1ccc(-c2ccc(-c3ccccc3-c3nc(-c4cccc(-c5ccccc5)c4)nc(-c4ccccc4-c4ccccc4)n3)cc2)cc1 ; c1ccc(-c2ccc(-c3ccccc3-c3nc(-c4ccccc4)nc(-c4cccc(-c5ccccc5)c4)n3)cc2)cc1 ; c1ccc(-c2ccc(-c3ccccc3-c3nc(-c4cccc(-c5ccccc5)c4)nc(-c4ccccc4-c4ccccc4-c4ccccc4)n3)cc2)cc1 ; c1ccc(-c2ccc(-c3ccccc3-c3nc(-c4cccc(-c5ccccc5)c4)nc(-c4cccc(-c5cccc(-c6ccccc6)c5)c4)n3)cc2)cc1]

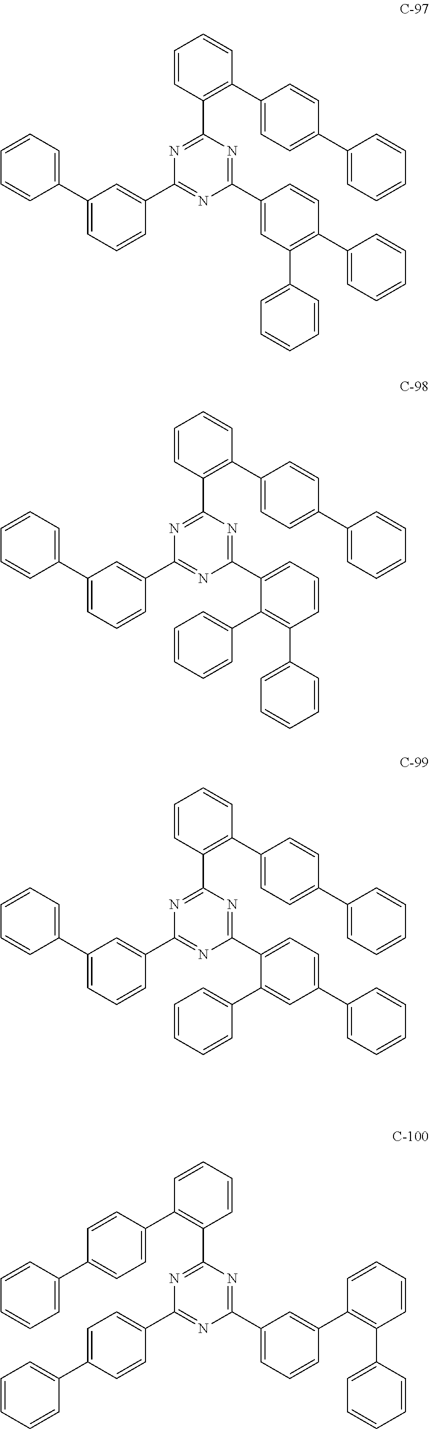 [SMILES: c1ccc(-c2ccc(-c3ccccc3-c3nc(-c4cccc(-c5ccccc5)c4)nc(-c4ccc(-c5ccccc5)c(-c5ccccc5)c4)n3)cc2)cc1 ; c1ccc(-c2ccc(-c3ccccc3-c3nc(-c4cccc(-c5ccccc5)c4)nc(-c4cccc(-c5ccccc5)c4-c4ccccc4)n3)cc2)cc1 ; c1ccc(-c2ccc(-c3ccccc3-c3nc(-c4cccc(-c5ccccc5)c4)nc(-c4ccc(-c5ccccc5)cc4-c4ccccc4)n3)cc2)cc1 ; c1ccc(-c2ccc(-c3nc(-c4cccc(-c5ccccc5-c5ccccc5)c4)nc(-c4ccccc4-c4ccc(-c5ccccc5)cc4)n3)cc2)cc1]

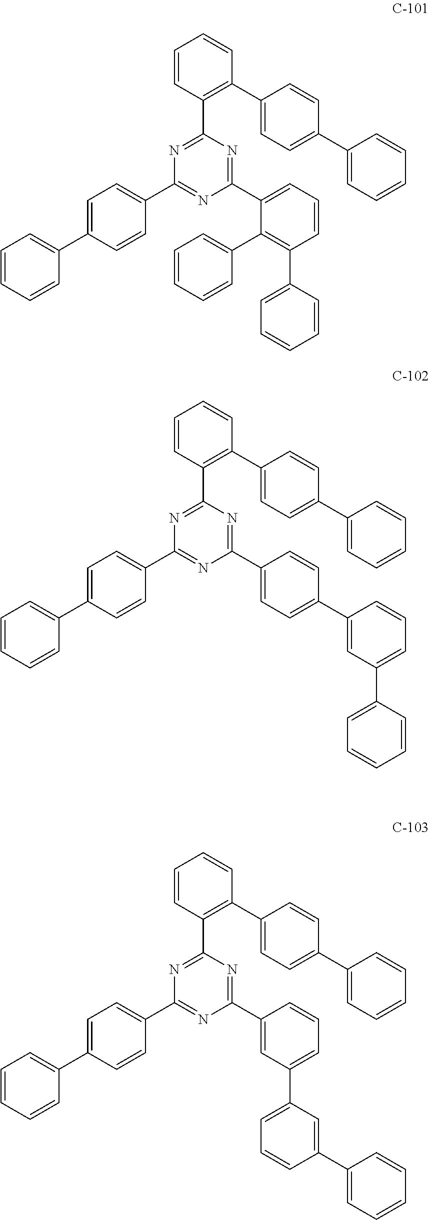 [SMILES: c1ccc(-c2ccc(-c3nc(-c4ccccc4-c4ccc(-c5ccccc5)cc4)nc(-c4cccc(-c5ccccc5)c4-c4ccccc4)n3)cc2)cc1 ; c1ccc(-c2ccc(-c3nc(-c4ccc(-c5cccc(-c6ccccc6)c5)cc4)nc(-c4ccccc4-c4ccc(-c5ccccc5)cc4)n3)cc2)cc1 ; c1ccc(-c2ccc(-c3nc(-c4cccc(-c5cccc(-c6ccccc6)c5)c4)nc(-c4ccccc4-c4ccc(-c5ccccc5)cc4)n3)cc2)cc1]

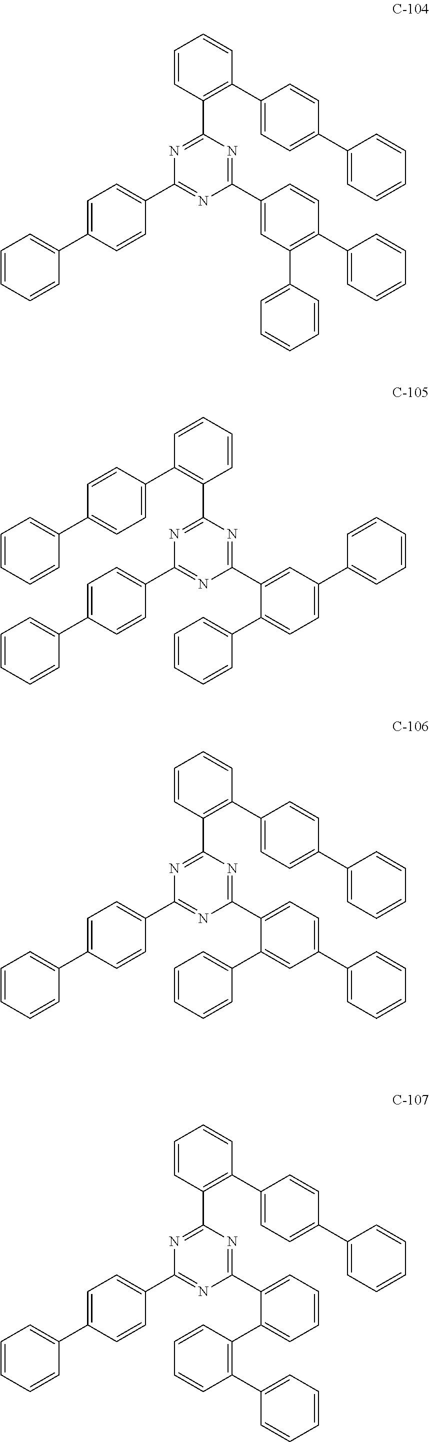 [SMILES: c1ccc(-c2ccc(-c3nc(-c4ccc(-c5ccccc5)c(-c5ccccc5)c4)nc(-c4ccccc4-c4ccc(-c5ccccc5)cc4)n3)cc2)cc1 ; c1ccc(-c2ccc(-c3nc(-c4ccccc4-c4ccc(-c5ccccc5)cc4)nc(-c4cc(-c5ccccc5)ccc4-c4ccccc4)n3)cc2)cc1 ; c1ccc(-c2ccc(-c3nc(-c4ccccc4-c4ccc(-c5ccccc5)cc4)nc(-c4ccc(-c5ccccc5)cc4-c4ccccc4)n3)cc2)cc1 ; c1ccc(-c2ccc(-c3nc(-c4ccccc4-c4ccc(-c5ccccc5)cc4)nc(-c4ccccc4-c4ccccc4-c4ccccc4)n3)cc2)cc1]

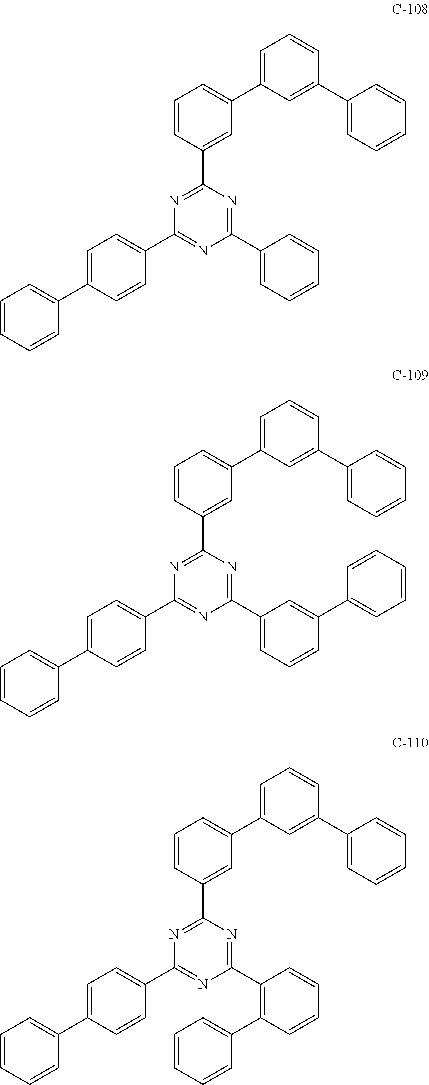 [SMILES: c1ccc(-c2ccc(-c3nc(-c4ccccc4)nc(-c4cccc(-c5cccc(-c6ccccc6)c5)c4)n3)cc2)cc1 ; c1ccc(-c2ccc(-c3nc(-c4cccc(-c5ccccc5)c4)nc(-c4cccc(-c5cccc(-c6ccccc6)c5)c4)n3)cc2)cc1 ; c1ccc(-c2ccc(-c3nc(-c4cccc(-c5cccc(-c6ccccc6)c5)c4)nc(-c4ccccc4-c4ccccc4)n3)cc2)cc1]

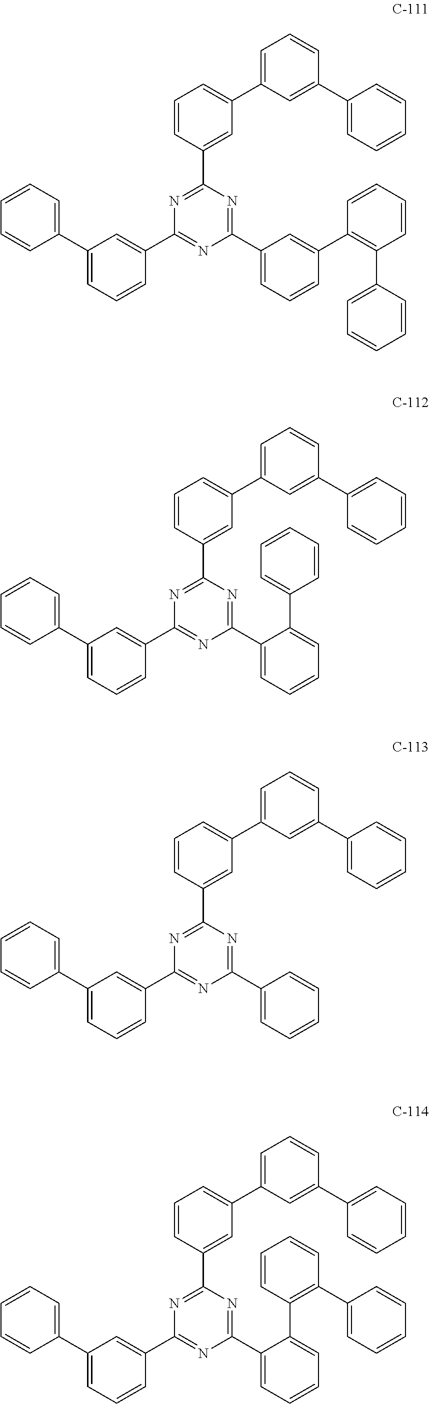 [SMILES: c1ccc(-c2cccc(-c3cccc(-c4nc(-c5cccc(-c6ccccc6)c5)nc(-c5cccc(-c6ccccc6-c6ccccc6)c5)n4)c3)c2)cc1 ; c1ccc(-c2cccc(-c3cccc(-c4nc(-c5cccc(-c6ccccc6)c5)nc(-c5ccccc5-c5ccccc5)n4)c3)c2)cc1 ; c1ccc(-c2cccc(-c3cccc(-c4nc(-c5ccccc5)nc(-c5cccc(-c6ccccc6)c5)n4)c3)c2)cc1 ; c1ccc(-c2cccc(-c3cccc(-c4nc(-c5cccc(-c6ccccc6)c5)nc(-c5ccccc5-c5ccccc5-c5ccccc5)n4)c3)c2)cc1]

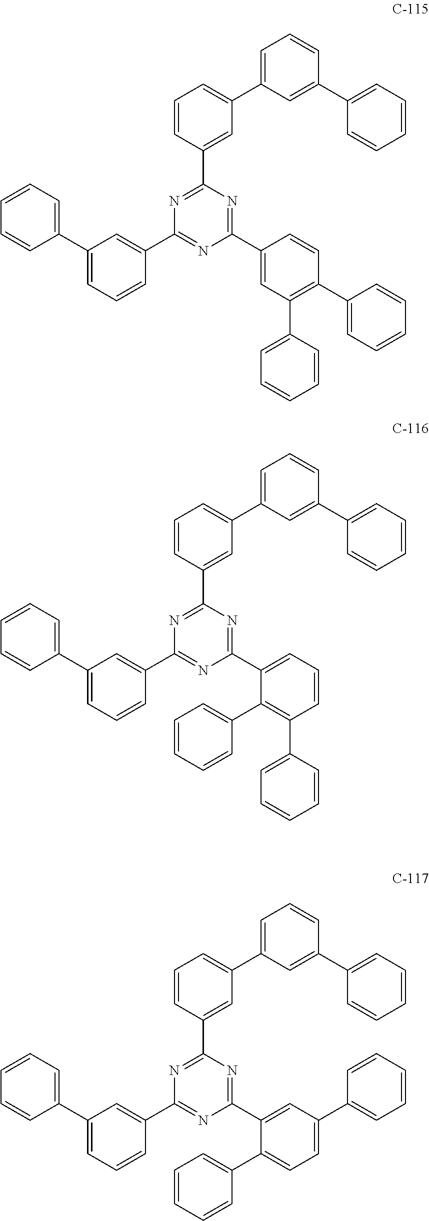 [SMILES: c1ccc(-c2cccc(-c3cccc(-c4nc(-c5cccc(-c6ccccc6)c5)nc(-c5ccc(-c6ccccc6)c(-c6ccccc6)c5)n4)c3)c2)cc1 ; c1ccc(-c2cccc(-c3cccc(-c4nc(-c5cccc(-c6ccccc6)c5)nc(-c5cccc(-c6ccccc6)c5-c5ccccc5)n4)c3)c2)cc1 ; c1ccc(-c2cccc(-c3cccc(-c4nc(-c5cccc(-c6ccccc6)c5)nc(-c5cc(-c6ccccc6)ccc5-c5ccccc5)n4)c3)c2)cc1]

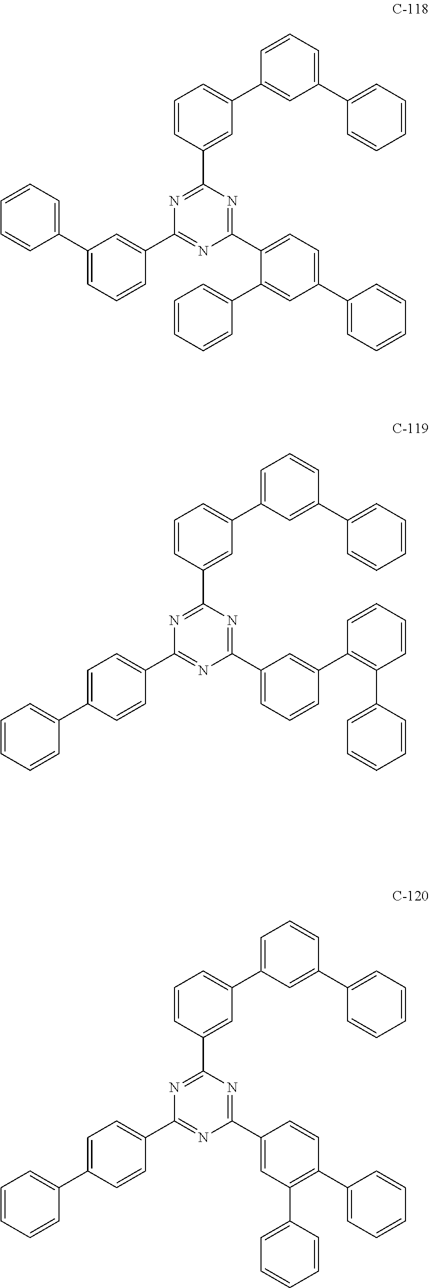 [SMILES: c1ccc(-c2cccc(-c3cccc(-c4nc(-c5cccc(-c6ccccc6)c5)nc(-c5ccc(-c6ccccc6)cc5-c5ccccc5)n4)c3)c2)cc1 ; c1ccc(-c2ccc(-c3nc(-c4cccc(-c5cccc(-c6ccccc6)c5)c4)nc(-c4cccc(-c5ccccc5-c5ccccc5)c4)n3)cc2)cc1 ; c1ccc(-c2ccc(-c3nc(-c4cccc(-c5cccc(-c6ccccc6)c5)c4)nc(-c4ccc(-c5ccccc5)c(-c5ccccc5)c4)n3)cc2)cc1]

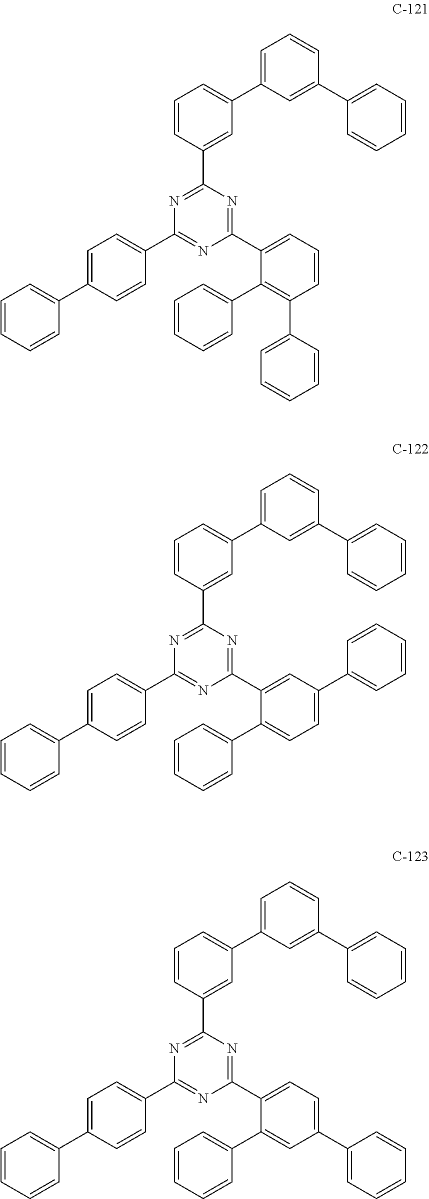 [SMILES: c1ccc(-c2ccc(-c3nc(-c4cccc(-c5cccc(-c6ccccc6)c5)c4)nc(-c4cccc(-c5ccccc5)c4-c4ccccc4)n3)cc2)cc1 ; c1ccc(-c2ccc(-c3nc(-c4cccc(-c5cccc(-c6ccccc6)c5)c4)nc(-c4cc(-c5ccccc5)ccc4-c4ccccc4)n3)cc2)cc1 ; c1ccc(-c2ccc(-c3nc(-c4cccc(-c5cccc(-c6ccccc6)c5)c4)nc(-c4ccc(-c5ccccc5)cc4-c4ccccc4)n3)cc2)cc1]

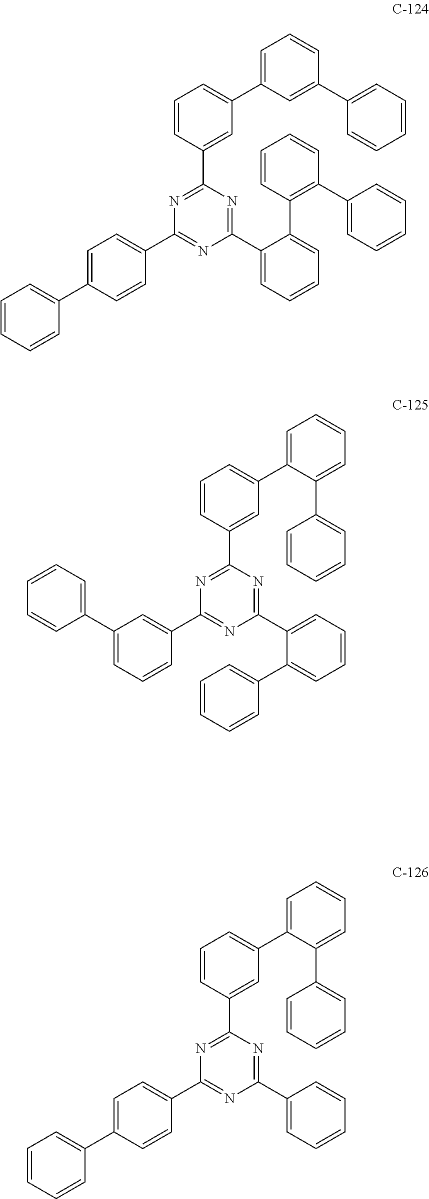 [SMILES: c1ccc(-c2ccc(-c3nc(-c4cccc(-c5cccc(-c6ccccc6)c5)c4)nc(-c4ccccc4-c4ccccc4-c4ccccc4)n3)cc2)cc1 ; c1ccc(-c2cccc(-c3nc(-c4cccc(-c5ccccc5-c5ccccc5)c4)nc(-c4ccccc4-c4ccccc4)n3)c2)cc1 ; c1ccc(-c2ccc(-c3nc(-c4ccccc4)nc(-c4cccc(-c5ccccc5-c5ccccc5)c4)n3)cc2)cc1]

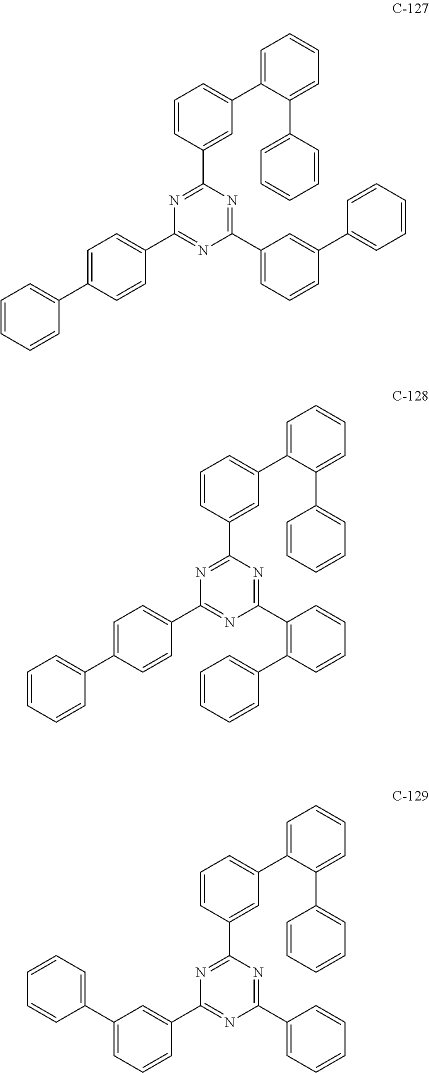 [SMILES: c1ccc(-c2ccc(-c3nc(-c4cccc(-c5ccccc5)c4)nc(-c4cccc(-c5ccccc5-c5ccccc5)c4)n3)cc2)cc1 ; c1ccc(-c2ccc(-c3nc(-c4cccc(-c5ccccc5-c5ccccc5)c4)nc(-c4ccccc4-c4ccccc4)n3)cc2)cc1 ; c1ccc(-c2cccc(-c3nc(-c4ccccc4)nc(-c4cccc(-c5ccccc5-c5ccccc5)c4)n3)c2)cc1]

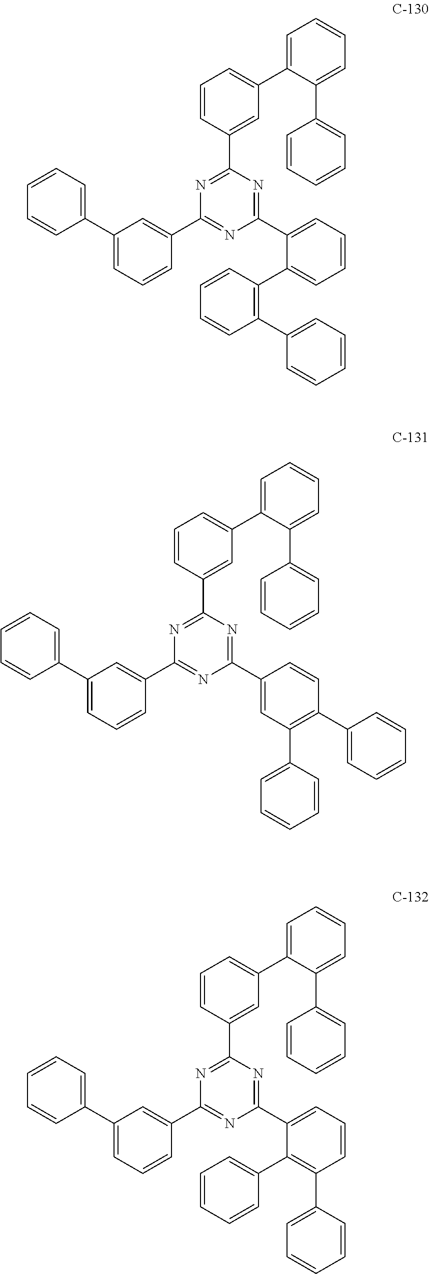 [SMILES: c1ccc(-c2cccc(-c3nc(-c4cccc(-c5ccccc5-c5ccccc5)c4)nc(-c4ccccc4-c4ccccc4-c4ccccc4)n3)c2)cc1 ; c1ccc(-c2cccc(-c3nc(-c4cccc(-c5ccccc5-c5ccccc5)c4)nc(-c4ccc(-c5ccccc5)c(-c5ccccc5)c4)n3)c2)cc1 ; c1ccc(-c2cccc(-c3nc(-c4cccc(-c5ccccc5-c5ccccc5)c4)nc(-c4cccc(-c5ccccc5)c4-c4ccccc4)n3)c2)cc1]

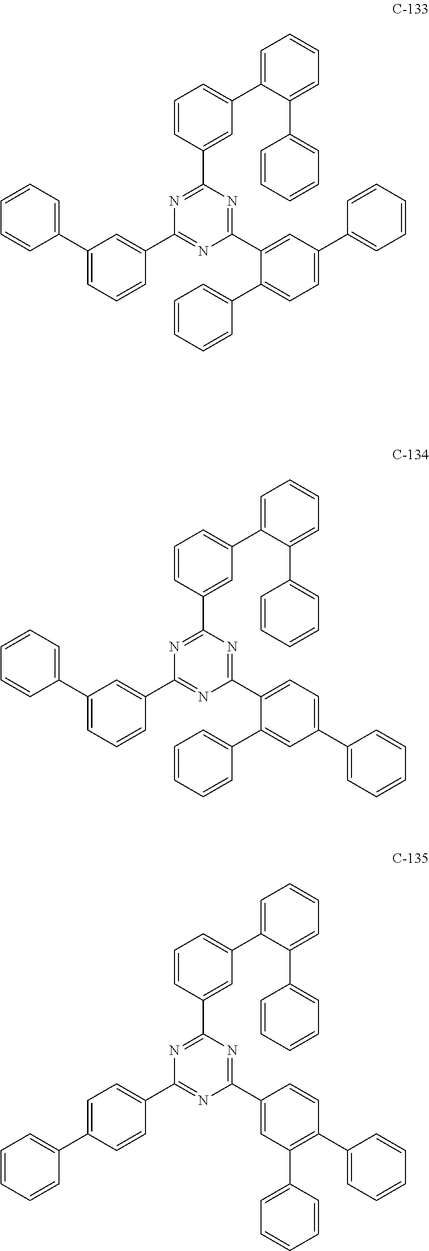 [SMILES: c1ccc(-c2cccc(-c3nc(-c4cccc(-c5ccccc5-c5ccccc5)c4)nc(-c4cc(-c5ccccc5)ccc4-c4ccccc4)n3)c2)cc1 ; c1ccc(-c2cccc(-c3nc(-c4cccc(-c5ccccc5-c5ccccc5)c4)nc(-c4ccc(-c5ccccc5)cc4-c4ccccc4)n3)c2)cc1 ; c1ccc(-c2ccc(-c3nc(-c4cccc(-c5ccccc5-c5ccccc5)c4)nc(-c4ccc(-c5ccccc5)c(-c5ccccc5)c4)n3)cc2)cc1]

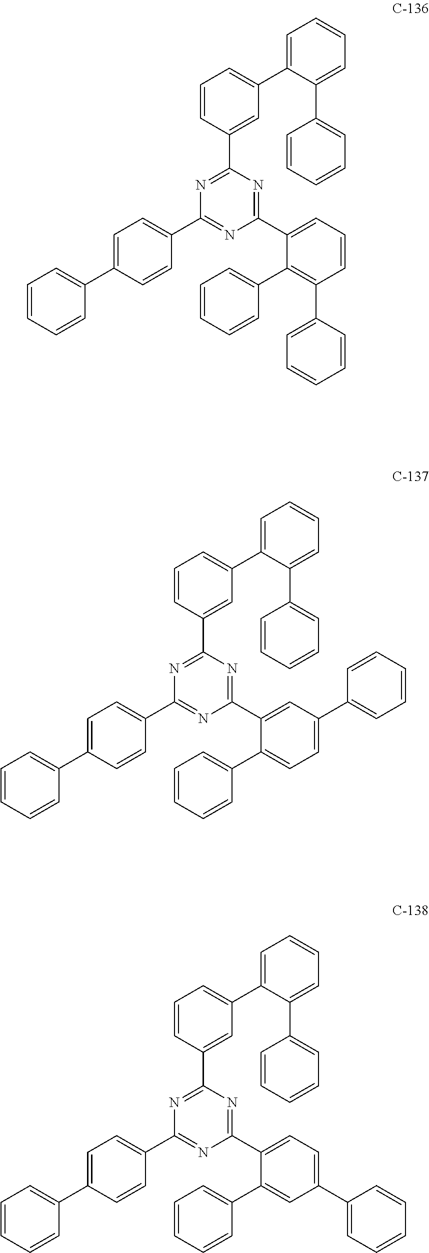 [SMILES: c1ccc(-c2ccc(-c3nc(-c4cccc(-c5ccccc5-c5ccccc5)c4)nc(-c4cccc(-c5ccccc5)c4-c4ccccc4)n3)cc2)cc1 ; c1ccc(-c2ccc(-c3nc(-c4cccc(-c5ccccc5-c5ccccc5)c4)nc(-c4cc(-c5ccccc5)ccc4-c4ccccc4)n3)cc2)cc1 ; c1ccc(-c2ccc(-c3nc(-c4cccc(-c5ccccc5-c5ccccc5)c4)nc(-c4ccc(-c5ccccc5)cc4-c4ccccc4)n3)cc2)cc1]

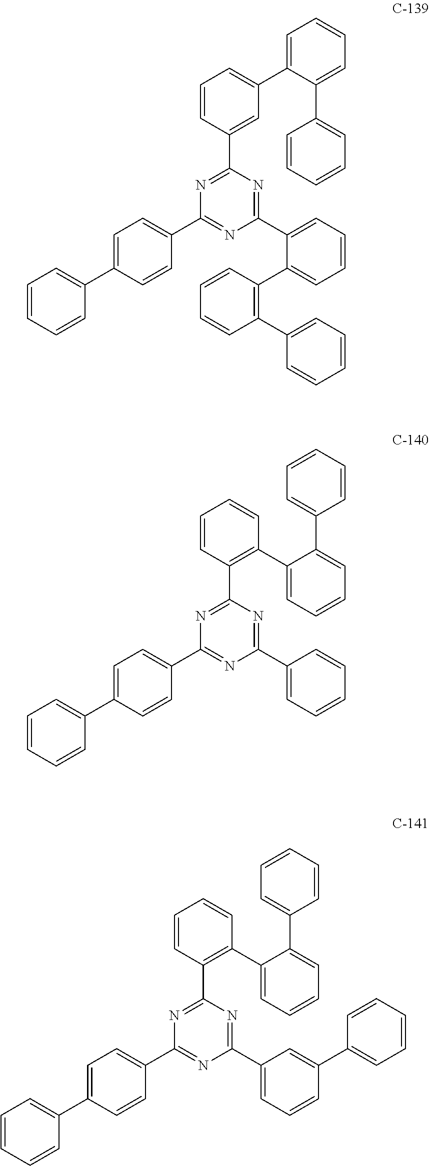 [SMILES: c1ccc(-c2ccc(-c3nc(-c4cccc(-c5ccccc5-c5ccccc5)c4)nc(-c4ccccc4-c4ccccc4-c4ccccc4)n3)cc2)cc1 ; c1ccc(-c2ccc(-c3nc(-c4ccccc4)nc(-c4ccccc4-c4ccccc4-c4ccccc4)n3)cc2)cc1 ; c1ccc(-c2ccc(-c3nc(-c4cccc(-c5ccccc5)c4)nc(-c4ccccc4-c4ccccc4-c4ccccc4)n3)cc2)cc1]

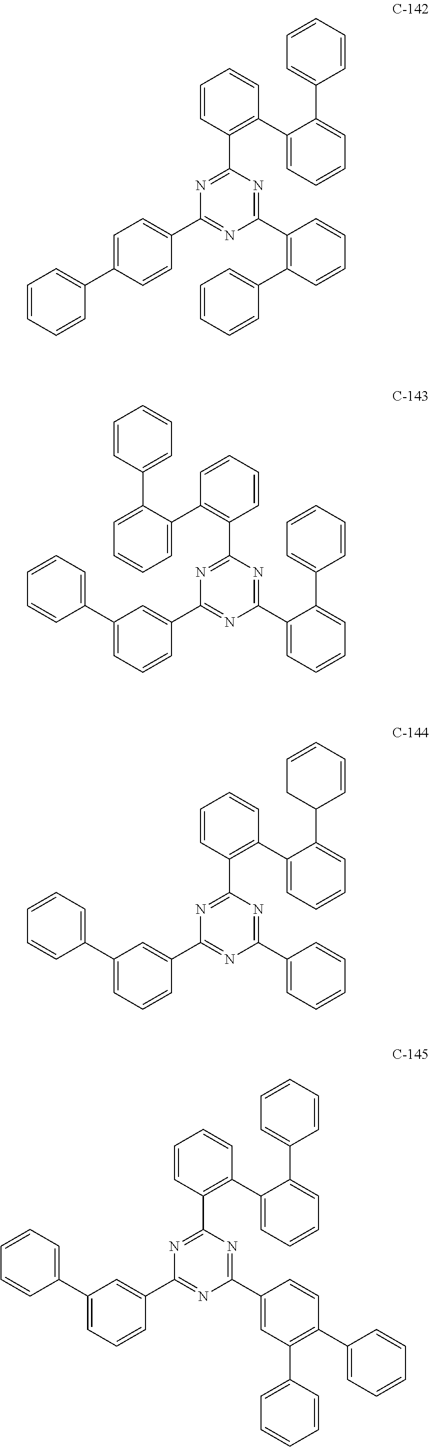 [SMILES: c1ccc(-c2ccc(-c3nc(-c4ccccc4-c4ccccc4)nc(-c4ccccc4-c4ccccc4-c4ccccc4)n3)cc2)cc1 ; c1ccc(-c2cccc(-c3nc(-c4ccccc4-c4ccccc4)nc(-c4ccccc4-c4ccccc4-c4ccccc4)n3)c2)cc1 ; C1=CCC(c2ccccc2-c2ccccc2-c2nc(-c3ccccc3)nc(-c3cccc(-c4ccccc4)c3)n2)C=C1 ; c1ccc(-c2cccc(-c3nc(-c4ccc(-c5ccccc5)c(-c5ccccc5)c4)nc(-c4ccccc4-c4ccccc4-c4ccccc4)n3)c2)cc1]

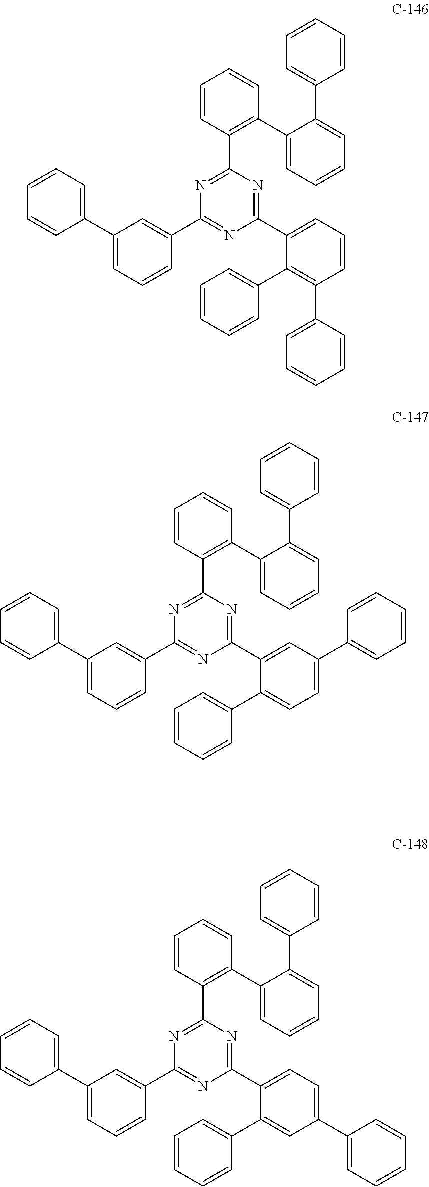 [SMILES: c1ccc(-c2cccc(-c3nc(-c4ccccc4-c4ccccc4-c4ccccc4)nc(-c4cccc(-c5ccccc5)c4-c4ccccc4)n3)c2)cc1 ; c1ccc(-c2cccc(-c3nc(-c4cc(-c5ccccc5)ccc4-c4ccccc4)nc(-c4ccccc4-c4ccccc4-c4ccccc4)n3)c2)cc1 ; c1ccc(-c2cccc(-c3nc(-c4ccc(-c5ccccc5)cc4-c4ccccc4)nc(-c4ccccc4-c4ccccc4-c4ccccc4)n3)c2)cc1]

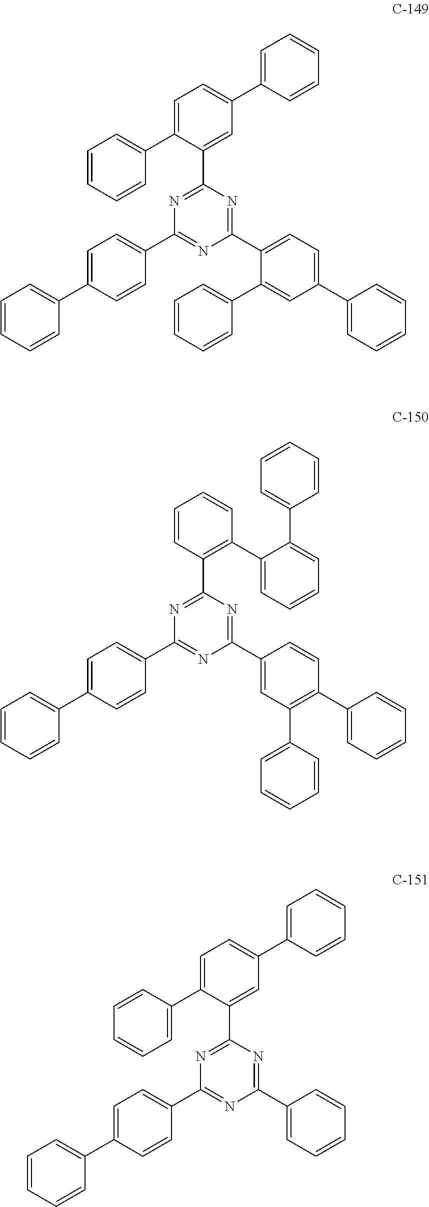 [SMILES: c1ccc(-c2ccc(-c3nc(-c4ccc(-c5ccccc5)cc4-c4ccccc4)nc(-c4cc(-c5ccccc5)ccc4-c4ccccc4)n3)cc2)cc1 ; c1ccc(-c2ccc(-c3nc(-c4ccc(-c5ccccc5)c(-c5ccccc5)c4)nc(-c4ccccc4-c4ccccc4-c4ccccc4)n3)cc2)cc1 ; c1ccc(-c2ccc(-c3nc(-c4ccccc4)nc(-c4cc(-c5ccccc5)ccc4-c4ccccc4)n3)cc2)cc1]

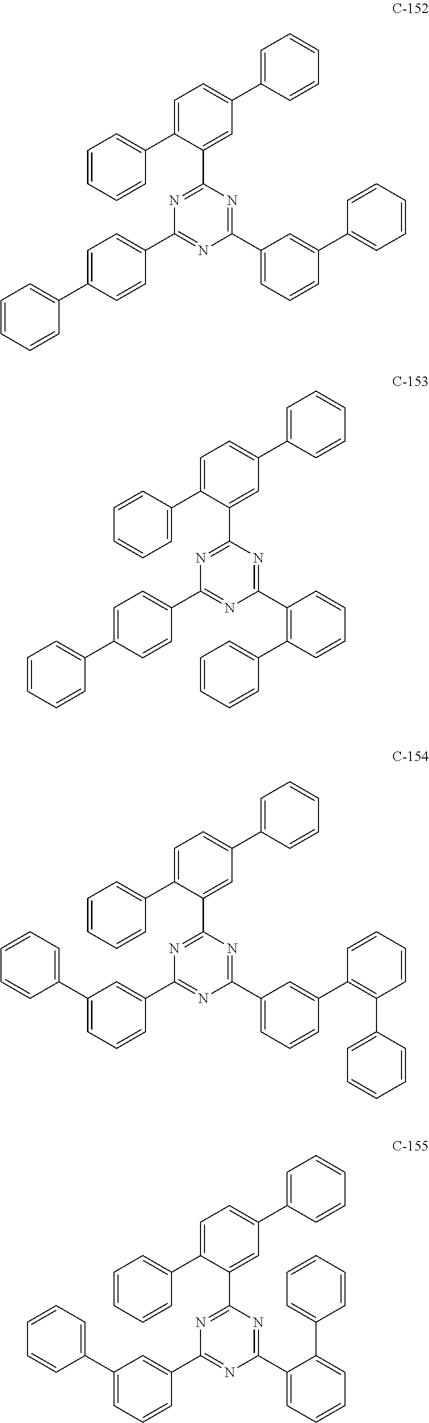 [SMILES: c1ccc(-c2ccc(-c3nc(-c4cccc(-c5ccccc5)c4)nc(-c4cc(-c5ccccc5)ccc4-c4ccccc4)n3)cc2)cc1 ; c1ccc(-c2ccc(-c3nc(-c4ccccc4-c4ccccc4)nc(-c4cc(-c5ccccc5)ccc4-c4ccccc4)n3)cc2)cc1 ; c1ccc(-c2cccc(-c3nc(-c4cccc(-c5ccccc5-c5ccccc5)c4)nc(-c4cc(-c5ccccc5)ccc4-c4ccccc4)n3)c2)cc1 ; c1ccc(-c2cccc(-c3nc(-c4ccccc4-c4ccccc4)nc(-c4cc(-c5ccccc5)ccc4-c4ccccc4)n3)c2)cc1]

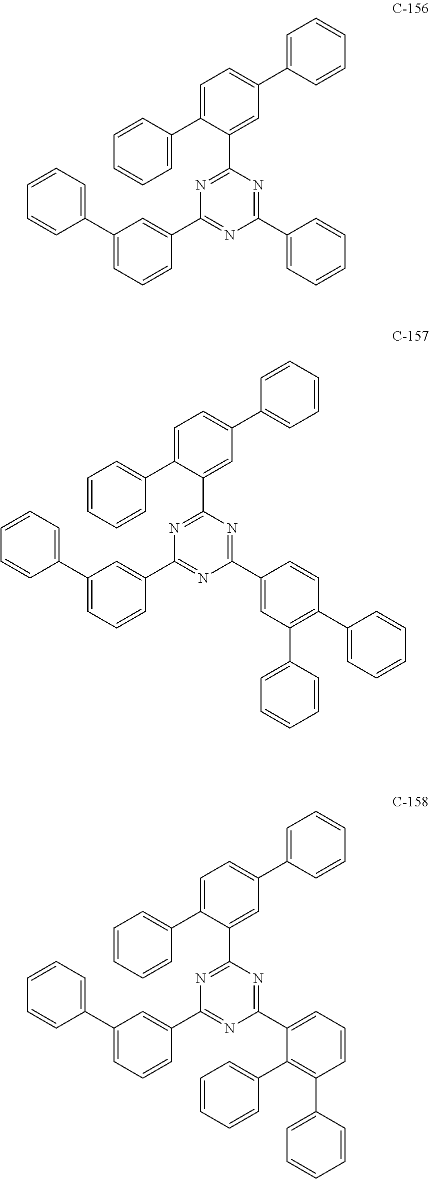 [SMILES: c1ccc(-c2cccc(-c3nc(-c4ccccc4)nc(-c4cc(-c5ccccc5)ccc4-c4ccccc4)n3)c2)cc1 ; c1ccc(-c2cccc(-c3nc(-c4ccc(-c5ccccc5)c(-c5ccccc5)c4)nc(-c4cc(-c5ccccc5)ccc4-c4ccccc4)n3)c2)cc1 ; c1ccc(-c2cccc(-c3nc(-c4cc(-c5ccccc5)ccc4-c4ccccc4)nc(-c4cccc(-c5ccccc5)c4-c4ccccc4)n3)c2)cc1]

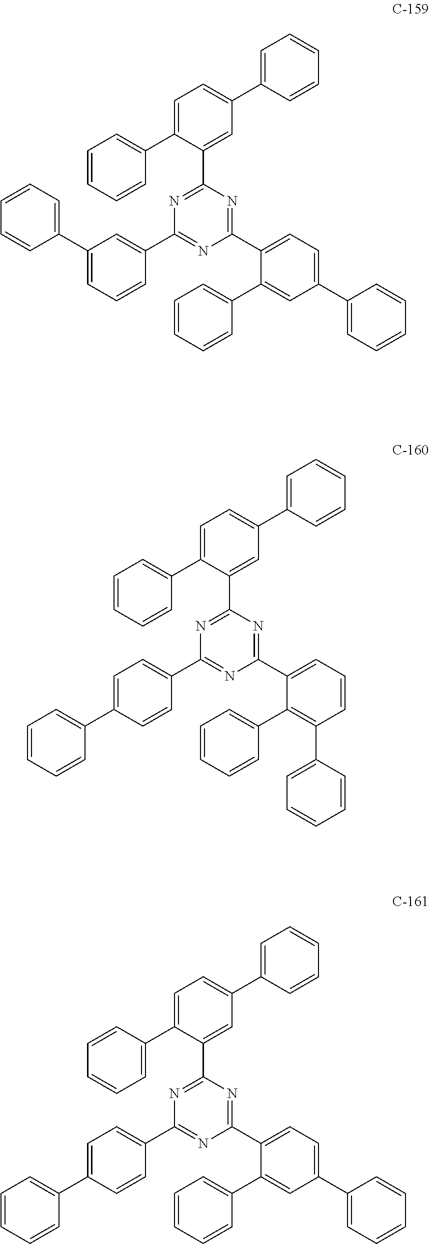 [SMILES: c1ccc(-c2cccc(-c3nc(-c4ccc(-c5ccccc5)cc4-c4ccccc4)nc(-c4cc(-c5ccccc5)ccc4-c4ccccc4)n3)c2)cc1 ; c1ccc(-c2ccc(-c3nc(-c4cc(-c5ccccc5)ccc4-c4ccccc4)nc(-c4cccc(-c5ccccc5)c4-c4ccccc4)n3)cc2)cc1 ; c1ccc(-c2ccc(-c3nc(-c4ccc(-c5ccccc5)cc4-c4ccccc4)nc(-c4cc(-c5ccccc5)ccc4-c4ccccc4)n3)cc2)cc1]

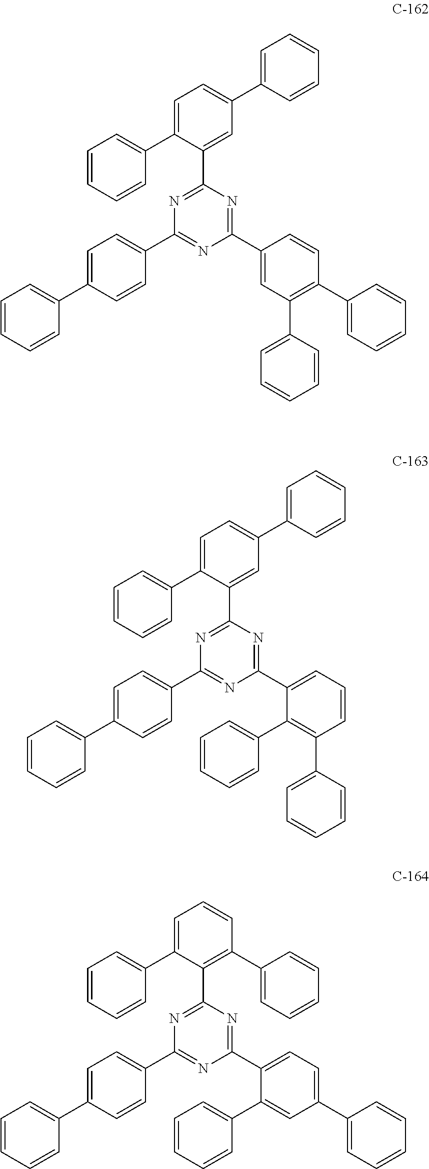 [SMILES: c1ccc(-c2ccc(-c3nc(-c4ccc(-c5ccccc5)c(-c5ccccc5)c4)nc(-c4cc(-c5ccccc5)ccc4-c4ccccc4)n3)cc2)cc1 ; c1ccc(-c2ccc(-c3nc(-c4cc(-c5ccccc5)ccc4-c4ccccc4)nc(-c4cccc(-c5ccccc5)c4-c4ccccc4)n3)cc2)cc1 ; c1ccc(-c2ccc(-c3nc(-c4ccc(-c5ccccc5)cc4-c4ccccc4)nc(-c4c(-c5ccccc5)cccc4-c4ccccc4)n3)cc2)cc1]

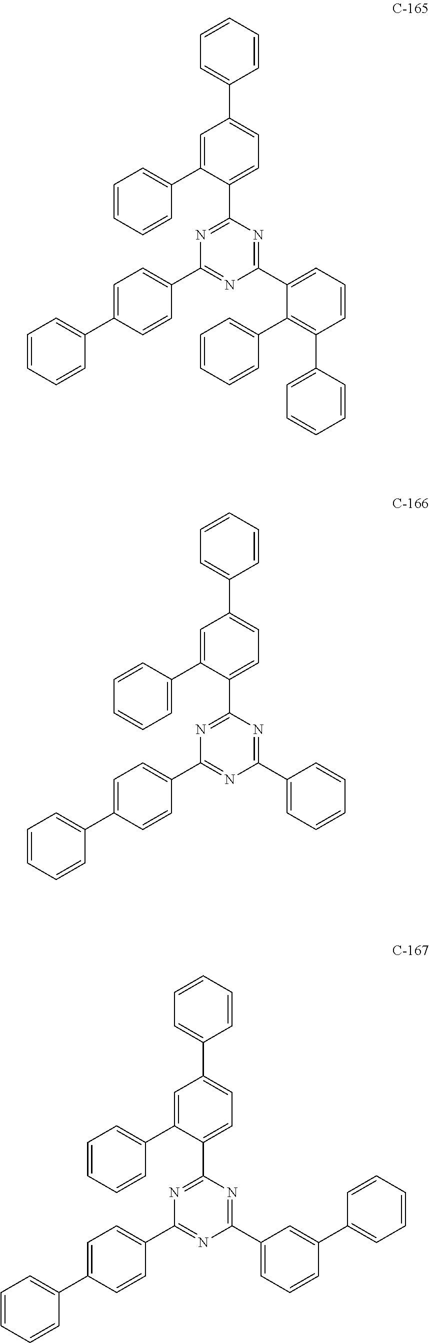 [SMILES: c1ccc(-c2ccc(-c3nc(-c4ccc(-c5ccccc5)cc4-c4ccccc4)nc(-c4cccc(-c5ccccc5)c4-c4ccccc4)n3)cc2)cc1 ; c1ccc(-c2ccc(-c3nc(-c4ccccc4)nc(-c4ccc(-c5ccccc5)cc4-c4ccccc4)n3)cc2)cc1 ; c1ccc(-c2ccc(-c3nc(-c4cccc(-c5ccccc5)c4)nc(-c4ccc(-c5ccccc5)cc4-c4ccccc4)n3)cc2)cc1]

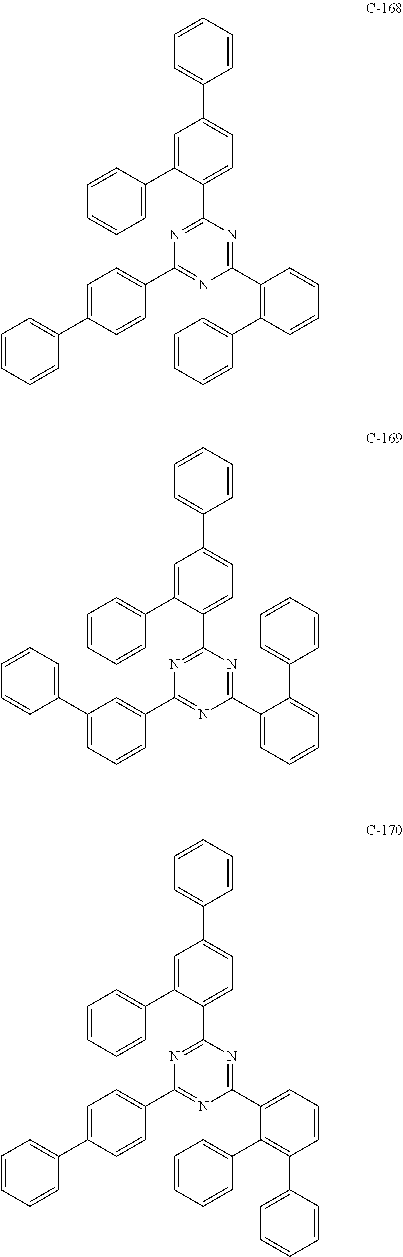 [SMILES: c1ccc(-c2ccc(-c3nc(-c4ccccc4-c4ccccc4)nc(-c4ccc(-c5ccccc5)cc4-c4ccccc4)n3)cc2)cc1 ; c1ccc(-c2cccc(-c3nc(-c4ccccc4-c4ccccc4)nc(-c4ccc(-c5ccccc5)cc4-c4ccccc4)n3)c2)cc1 ; c1ccc(-c2ccc(-c3nc(-c4ccc(-c5ccccc5)cc4-c4ccccc4)nc(-c4cccc(-c5ccccc5)c4-c4ccccc4)n3)cc2)cc1]

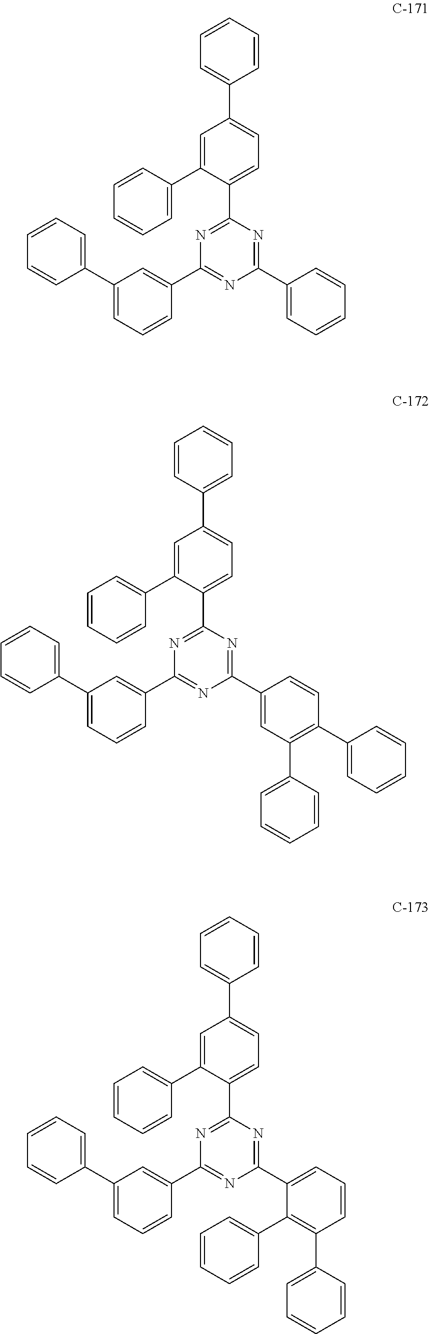 [SMILES: c1ccc(-c2cccc(-c3nc(-c4ccccc4)nc(-c4ccc(-c5ccccc5)cc4-c4ccccc4)n3)c2)cc1 ; c1ccc(-c2cccc(-c3nc(-c4ccc(-c5ccccc5)c(-c5ccccc5)c4)nc(-c4ccc(-c5ccccc5)cc4-c4ccccc4)n3)c2)cc1 ; c1ccc(-c2cccc(-c3nc(-c4ccc(-c5ccccc5)cc4-c4ccccc4)nc(-c4cccc(-c5ccccc5)c4-c4ccccc4)n3)c2)cc1]

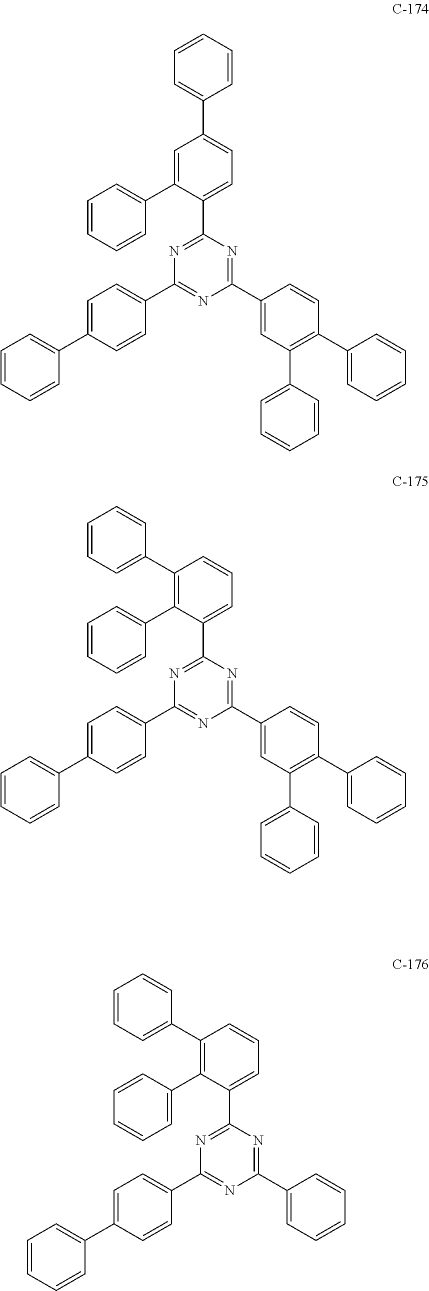 [SMILES: c1ccc(-c2ccc(-c3nc(-c4ccc(-c5ccccc5)c(-c5ccccc5)c4)nc(-c4ccc(-c5ccccc5)cc4-c4ccccc4)n3)cc2)cc1 ; c1ccc(-c2ccc(-c3nc(-c4ccc(-c5ccccc5)c(-c5ccccc5)c4)nc(-c4cccc(-c5ccccc5)c4-c4ccccc4)n3)cc2)cc1 ; c1ccc(-c2ccc(-c3nc(-c4ccccc4)nc(-c4cccc(-c5ccccc5)c4-c4ccccc4)n3)cc2)cc1]

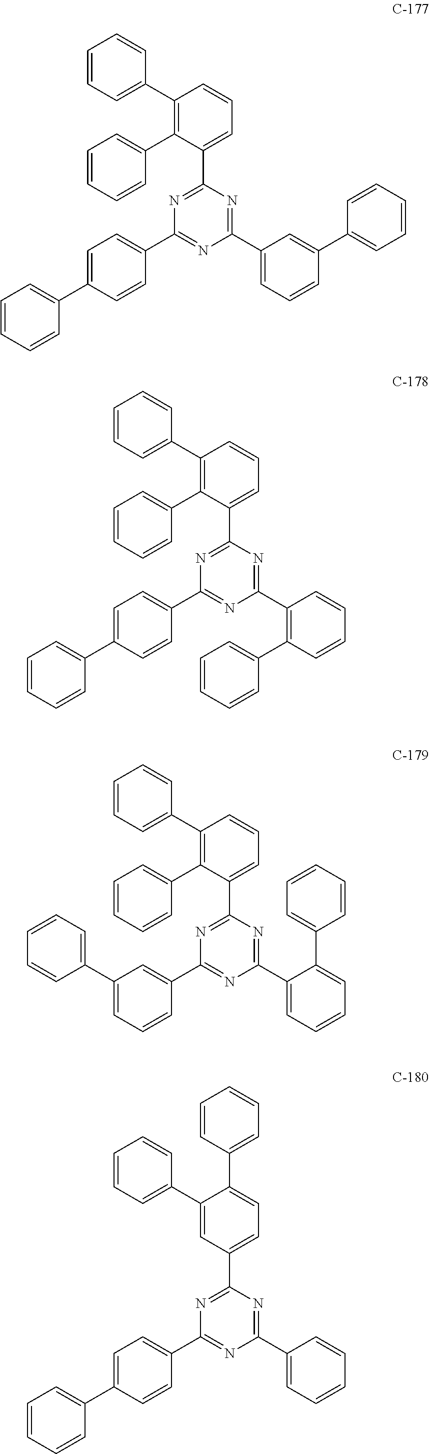 [SMILES: c1ccc(-c2ccc(-c3nc(-c4cccc(-c5ccccc5)c4)nc(-c4cccc(-c5ccccc5)c4-c4ccccc4)n3)cc2)cc1 ; c1ccc(-c2ccc(-c3nc(-c4ccccc4-c4ccccc4)nc(-c4cccc(-c5ccccc5)c4-c4ccccc4)n3)cc2)cc1 ; c1ccc(-c2cccc(-c3nc(-c4ccccc4-c4ccccc4)nc(-c4cccc(-c5ccccc5)c4-c4ccccc4)n3)c2)cc1 ; c1ccc(-c2ccc(-c3nc(-c4ccccc4)nc(-c4ccc(-c5ccccc5)c(-c5ccccc5)c4)n3)cc2)cc1]

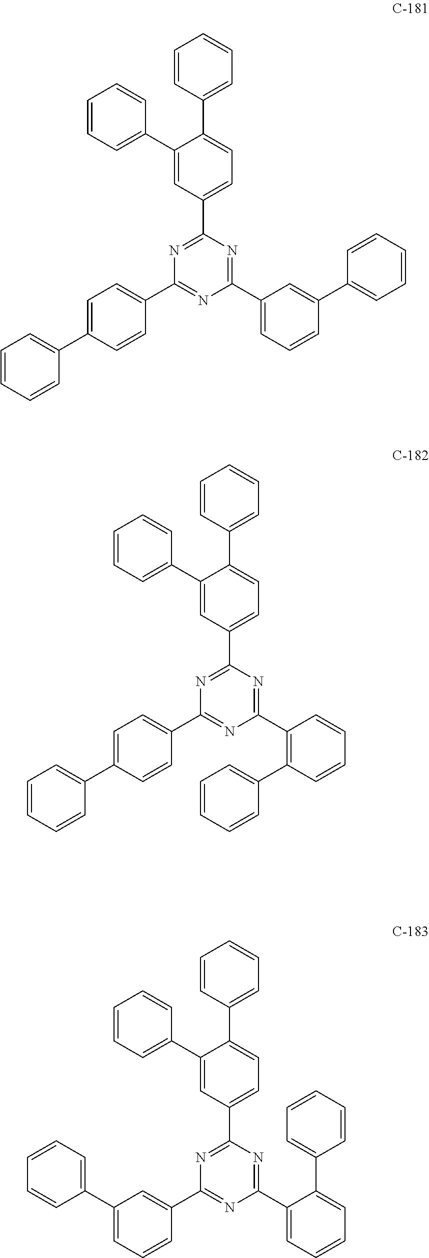 [SMILES: c1ccc(-c2ccc(-c3nc(-c4cccc(-c5ccccc5)c4)nc(-c4ccc(-c5ccccc5)c(-c5ccccc5)c4)n3)cc2)cc1 ; c1ccc(-c2ccc(-c3nc(-c4ccc(-c5ccccc5)c(-c5ccccc5)c4)nc(-c4ccccc4-c4ccccc4)n3)cc2)cc1 ; c1ccc(-c2cccc(-c3nc(-c4ccc(-c5ccccc5)c(-c5ccccc5)c4)nc(-c4ccccc4-c4ccccc4)n3)c2)cc1]

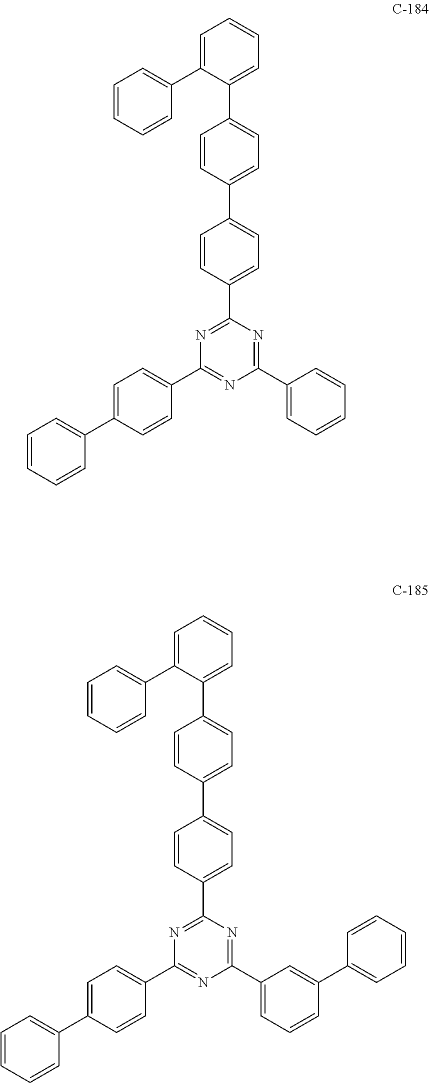 [SMILES: c1ccc(-c2ccc(-c3nc(-c4ccccc4)nc(-c4ccc(-c5ccc(-c6ccccc6-c6ccccc6)cc5)cc4)n3)cc2)cc1 ; c1ccc(-c2ccc(-c3nc(-c4ccc(-c5ccc(-c6ccccc6-c6ccccc6)cc5)cc4)nc(-c4cccc(-c5ccccc5)c4)n3)cc2)cc1]

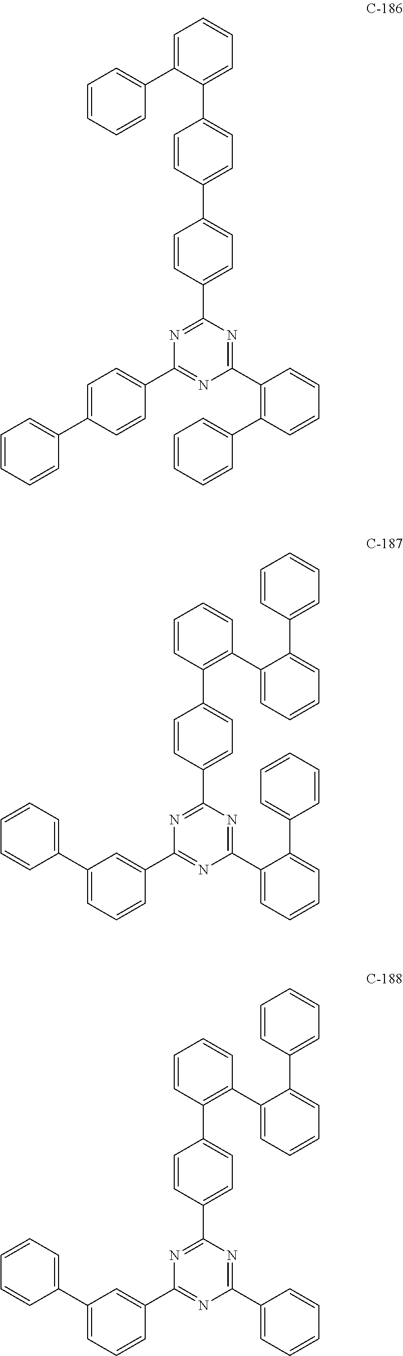 [SMILES: c1ccc(-c2ccc(-c3nc(-c4ccc(-c5ccc(-c6ccccc6-c6ccccc6)cc5)cc4)nc(-c4ccccc4-c4ccccc4)n3)cc2)cc1 ; c1ccc(-c2cccc(-c3nc(-c4ccc(-c5ccccc5-c5ccccc5-c5ccccc5)cc4)nc(-c4ccccc4-c4ccccc4)n3)c2)cc1 ; c1ccc(-c2cccc(-c3nc(-c4ccccc4)nc(-c4ccc(-c5ccccc5-c5ccccc5-c5ccccc5)cc4)n3)c2)cc1]

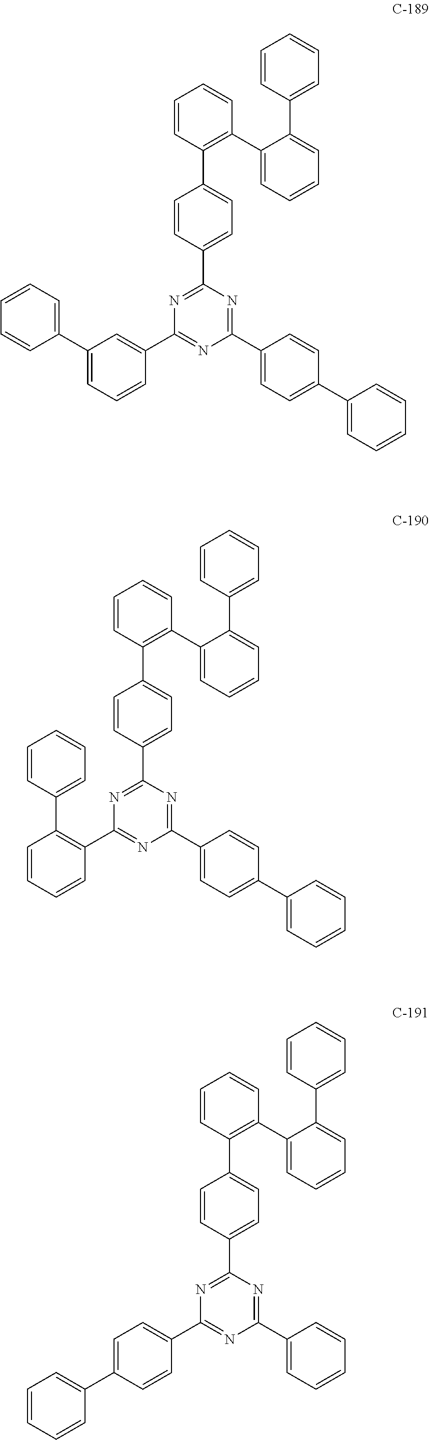 [SMILES: c1ccc(-c2ccc(-c3nc(-c4ccc(-c5ccccc5-c5ccccc5-c5ccccc5)cc4)nc(-c4cccc(-c5ccccc5)c4)n3)cc2)cc1 ; c1ccc(-c2ccc(-c3nc(-c4ccc(-c5ccccc5-c5ccccc5-c5ccccc5)cc4)nc(-c4ccccc4-c4ccccc4)n3)cc2)cc1 ; c1ccc(-c2ccc(-c3nc(-c4ccccc4)nc(-c4ccc(-c5ccccc5-c5ccccc5-c5ccccc5)cc4)n3)cc2)cc1]

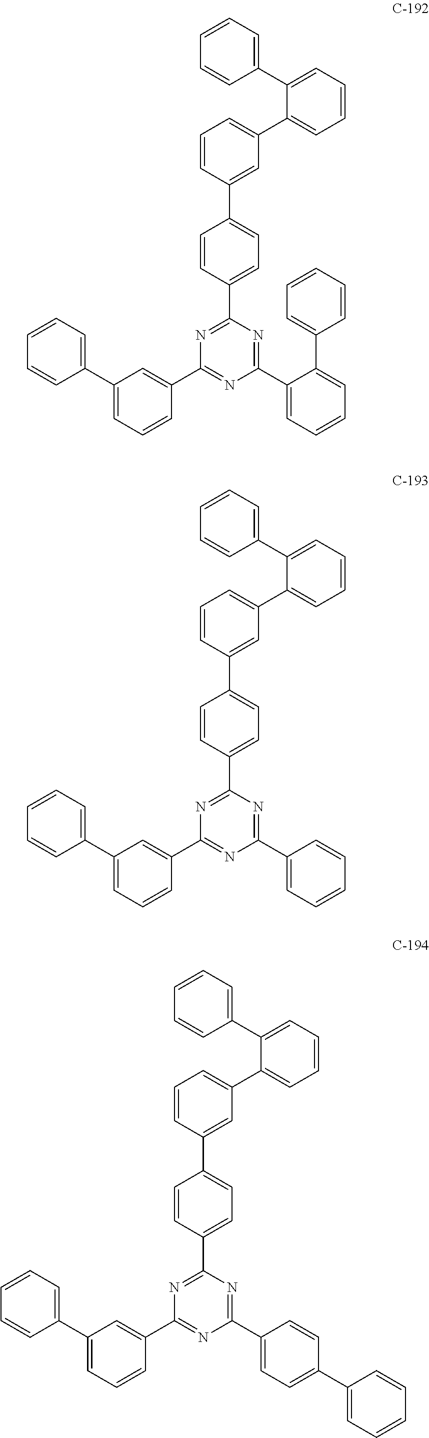 [SMILES: c1ccc(-c2cccc(-c3nc(-c4ccc(-c5cccc(-c6ccccc6-c6ccccc6)c5)cc4)nc(-c4ccccc4-c4ccccc4)n3)c2)cc1 ; c1ccc(-c2cccc(-c3nc(-c4ccccc4)nc(-c4ccc(-c5cccc(-c6ccccc6-c6ccccc6)c5)cc4)n3)c2)cc1 ; c1ccc(-c2ccc(-c3nc(-c4ccc(-c5cccc(-c6ccccc6-c6ccccc6)c5)cc4)nc(-c4cccc(-c5ccccc5)c4)n3)cc2)cc1]

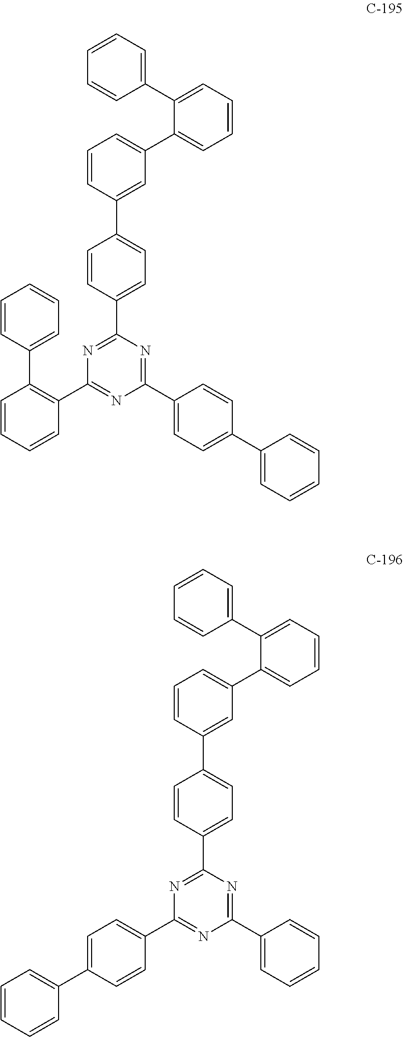 [SMILES: c1ccc(-c2ccc(-c3nc(-c4ccc(-c5cccc(-c6ccccc6-c6ccccc6)c5)cc4)nc(-c4ccccc4-c4ccccc4)n3)cc2)cc1 ; c1ccc(-c2ccc(-c3nc(-c4ccccc4)nc(-c4ccc(-c5cccc(-c6ccccc6-c6ccccc6)c5)cc4)n3)cc2)cc1]

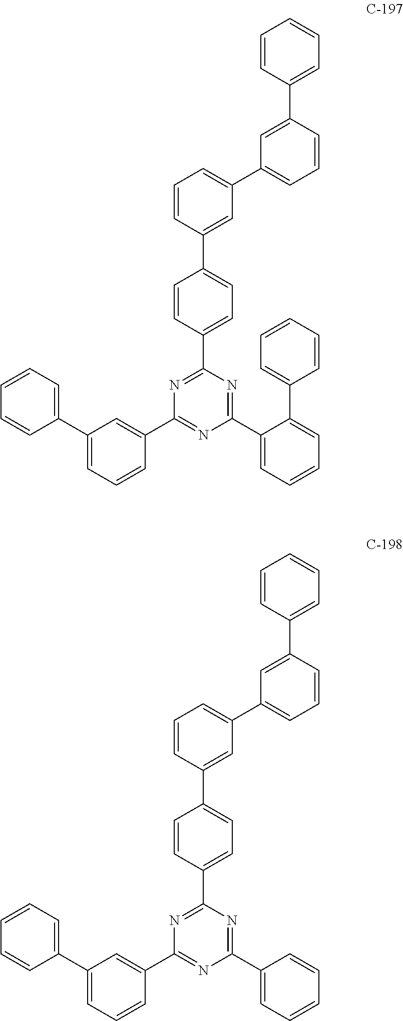 [SMILES: c1ccc(-c2cccc(-c3cccc(-c4ccc(-c5nc(-c6cccc(-c7ccccc7)c6)nc(-c6ccccc6-c6ccccc6)n5)cc4)c3)c2)cc1 ; c1ccc(-c2cccc(-c3cccc(-c4ccc(-c5nc(-c6ccccc6)nc(-c6cccc(-c7ccccc7)c6)n5)cc4)c3)c2)cc1]

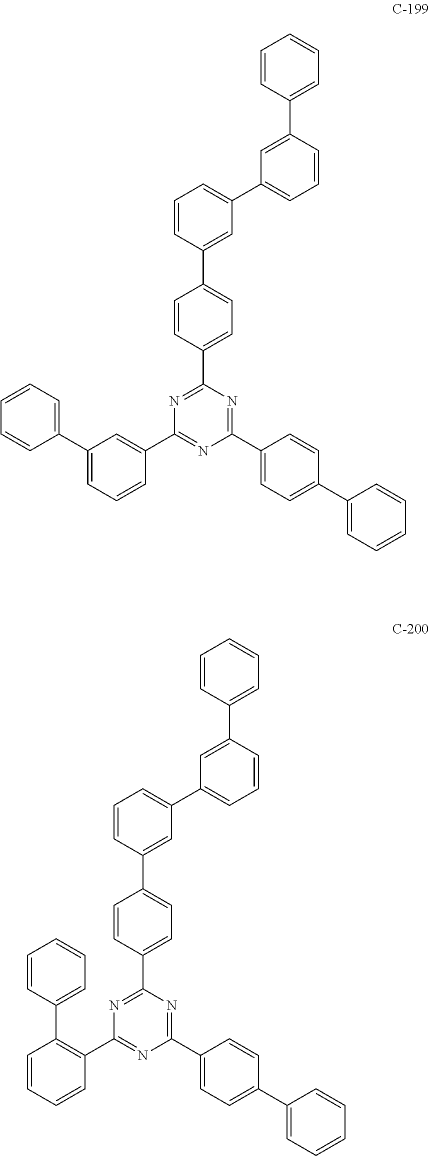 [SMILES: c1ccc(-c2ccc(-c3nc(-c4ccc(-c5cccc(-c6cccc(-c7ccccc7)c6)c5)cc4)nc(-c4cccc(-c5ccccc5)c4)n3)cc2)cc1 ; c1ccc(-c2ccc(-c3nc(-c4ccc(-c5cccc(-c6cccc(-c7ccccc7)c6)c5)cc4)nc(-c4ccccc4-c4ccccc4)n3)cc2)cc1]

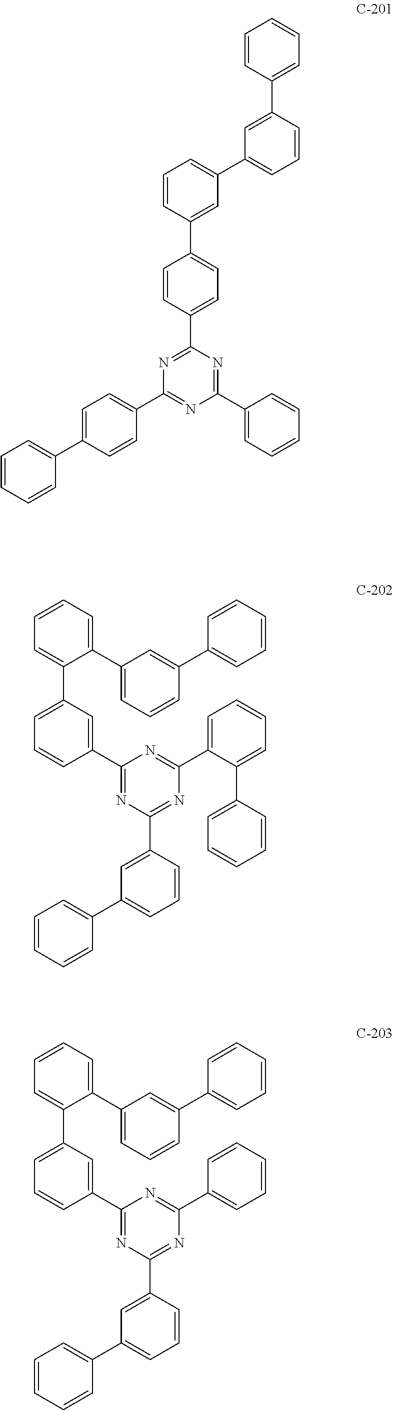 [SMILES: c1ccc(-c2ccc(-c3nc(-c4ccccc4)nc(-c4ccc(-c5cccc(-c6cccc(-c7ccccc7)c6)c5)cc4)n3)cc2)cc1 ; c1ccc(-c2cccc(-c3nc(-c4cccc(-c5ccccc5-c5cccc(-c6ccccc6)c5)c4)nc(-c4ccccc4-c4ccccc4)n3)c2)cc1 ; c1ccc(-c2cccc(-c3nc(-c4ccccc4)nc(-c4cccc(-c5ccccc5-c5cccc(-c6ccccc6)c5)c4)n3)c2)cc1]

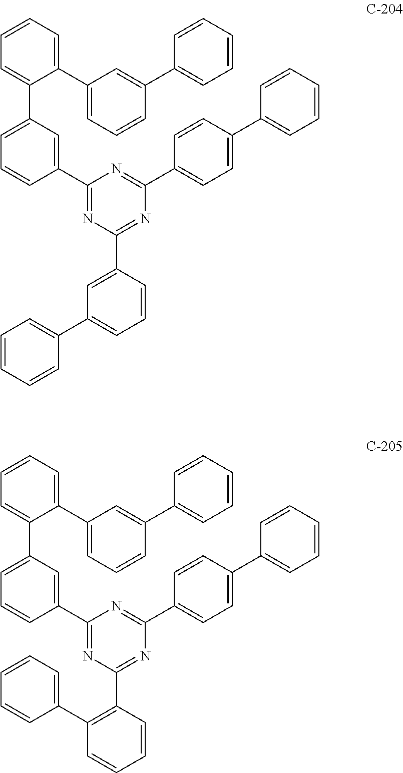 [SMILES: c1ccc(-c2ccc(-c3nc(-c4cccc(-c5ccccc5)c4)nc(-c4cccc(-c5ccccc5-c5cccc(-c6ccccc6)c5)c4)n3)cc2)cc1 ; c1ccc(-c2ccc(-c3nc(-c4cccc(-c5ccccc5-c5cccc(-c6ccccc6)c5)c4)nc(-c4ccccc4-c4ccccc4)n3)cc2)cc1]

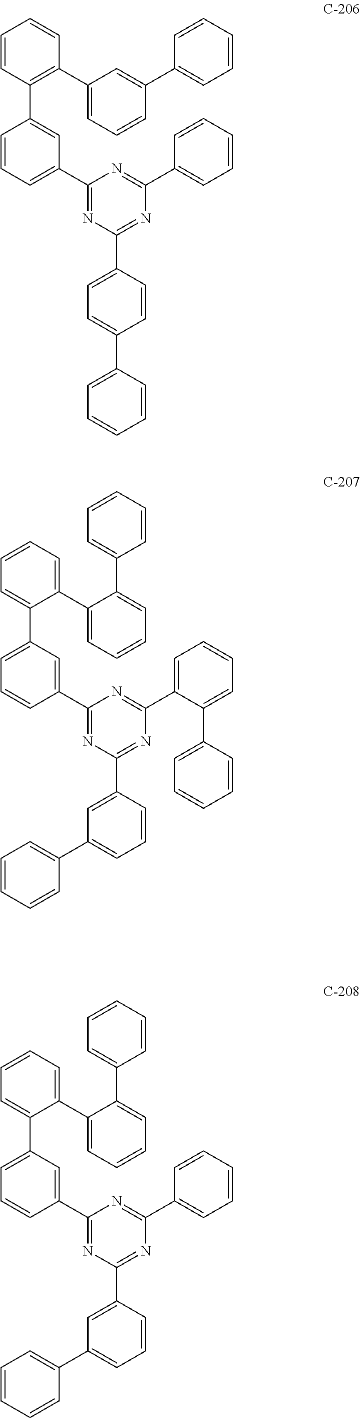 [SMILES: c1ccc(-c2ccc(-c3nc(-c4ccccc4)nc(-c4cccc(-c5ccccc5-c5cccc(-c6ccccc6)c5)c4)n3)cc2)cc1 ; c1ccc(-c2cccc(-c3nc(-c4cccc(-c5ccccc5-c5ccccc5-c5ccccc5)c4)nc(-c4ccccc4-c4ccccc4)n3)c2)cc1 ; c1ccc(-c2cccc(-c3nc(-c4ccccc4)nc(-c4cccc(-c5ccccc5-c5ccccc5-c5ccccc5)c4)n3)c2)cc1]

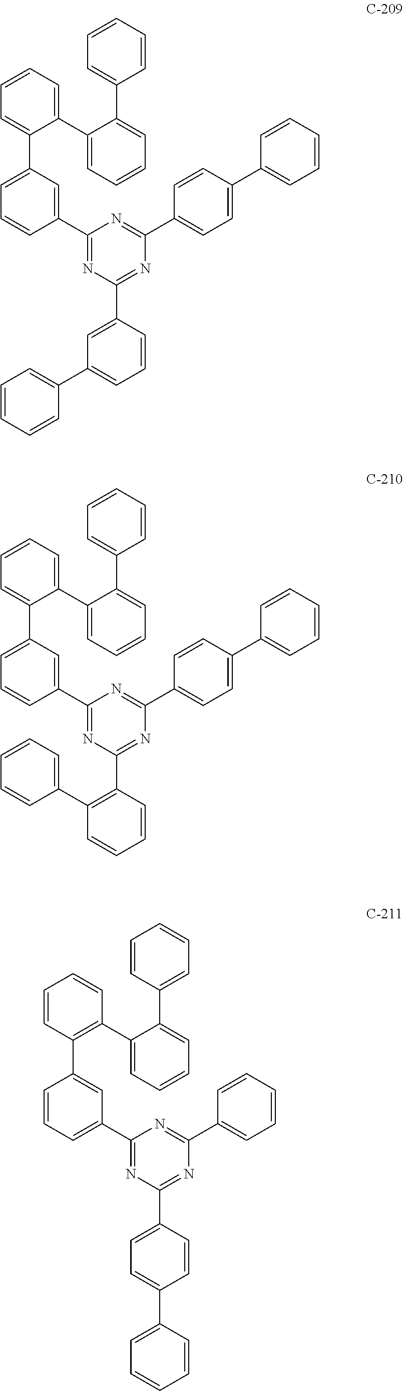 [SMILES: c1ccc(-c2ccc(-c3nc(-c4cccc(-c5ccccc5)c4)nc(-c4cccc(-c5ccccc5-c5ccccc5-c5ccccc5)c4)n3)cc2)cc1 ; c1ccc(-c2ccc(-c3nc(-c4cccc(-c5ccccc5-c5ccccc5-c5ccccc5)c4)nc(-c4ccccc4-c4ccccc4)n3)cc2)cc1 ; c1ccc(-c2ccc(-c3nc(-c4ccccc4)nc(-c4cccc(-c5ccccc5-c5ccccc5-c5ccccc5)c4)n3)cc2)cc1]

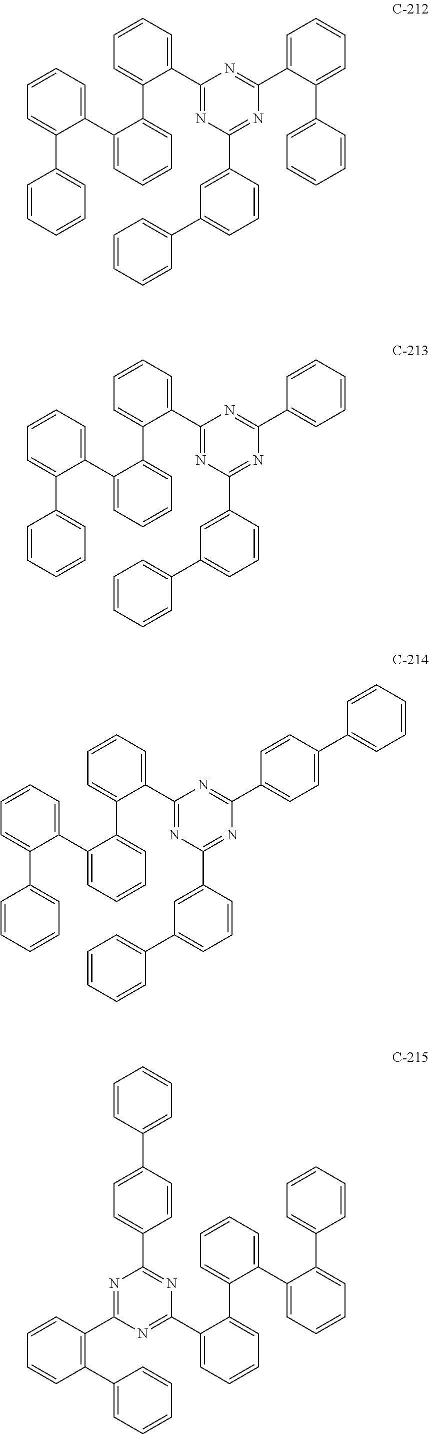 [SMILES: c1ccc(-c2cccc(-c3nc(-c4ccccc4-c4ccccc4)nc(-c4ccccc4-c4ccccc4-c4ccccc4-c4ccccc4)n3)c2)cc1 ; c1ccc(-c2cccc(-c3nc(-c4ccccc4)nc(-c4ccccc4-c4ccccc4-c4ccccc4-c4ccccc4)n3)c2)cc1 ; c1ccc(-c2ccc(-c3nc(-c4cccc(-c5ccccc5)c4)nc(-c4ccccc4-c4ccccc4-c4ccccc4-c4ccccc4)n3)cc2)cc1 ; c1ccc(-c2ccc(-c3nc(-c4ccccc4-c4ccccc4)nc(-c4ccccc4-c4ccccc4-c4ccccc4-c4ccccc4)n3)cc2)cc1]

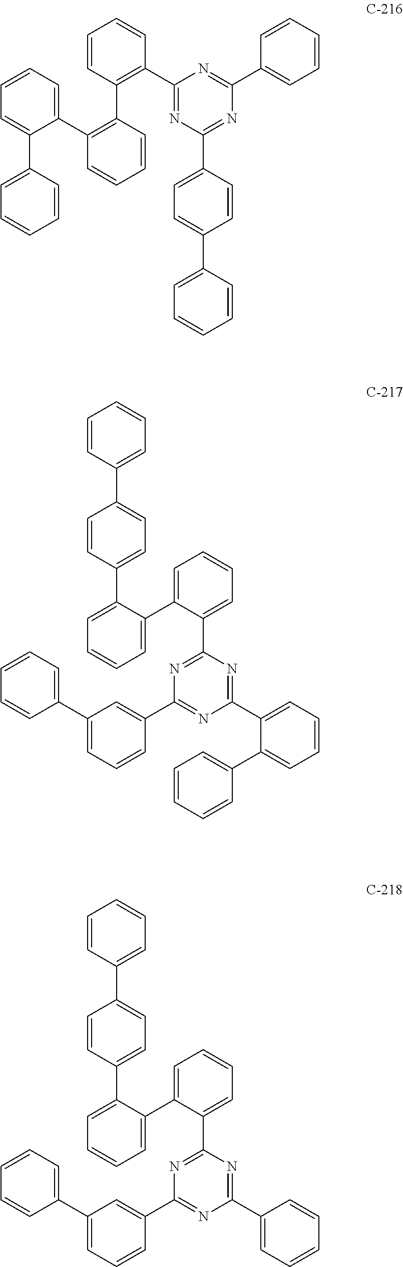 [SMILES: c1ccc(-c2ccc(-c3nc(-c4ccccc4)nc(-c4ccccc4-c4ccccc4-c4ccccc4-c4ccccc4)n3)cc2)cc1 ; c1ccc(-c2ccc(-c3ccccc3-c3ccccc3-c3nc(-c4cccc(-c5ccccc5)c4)nc(-c4ccccc4-c4ccccc4)n3)cc2)cc1 ; c1ccc(-c2ccc(-c3ccccc3-c3ccccc3-c3nc(-c4ccccc4)nc(-c4cccc(-c5ccccc5)c4)n3)cc2)cc1]

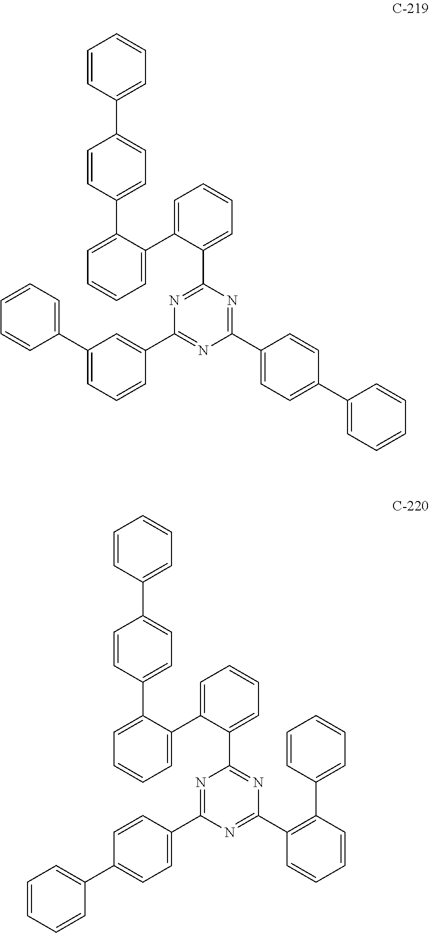 [SMILES: c1ccc(-c2ccc(-c3nc(-c4cccc(-c5ccccc5)c4)nc(-c4ccccc4-c4ccccc4-c4ccc(-c5ccccc5)cc4)n3)cc2)cc1 ; c1ccc(-c2ccc(-c3nc(-c4ccccc4-c4ccccc4)nc(-c4ccccc4-c4ccccc4-c4ccc(-c5ccccc5)cc4)n3)cc2)cc1]

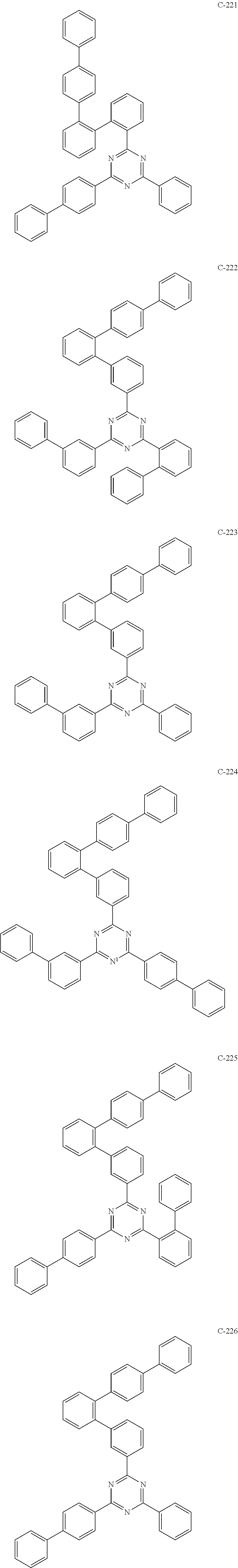

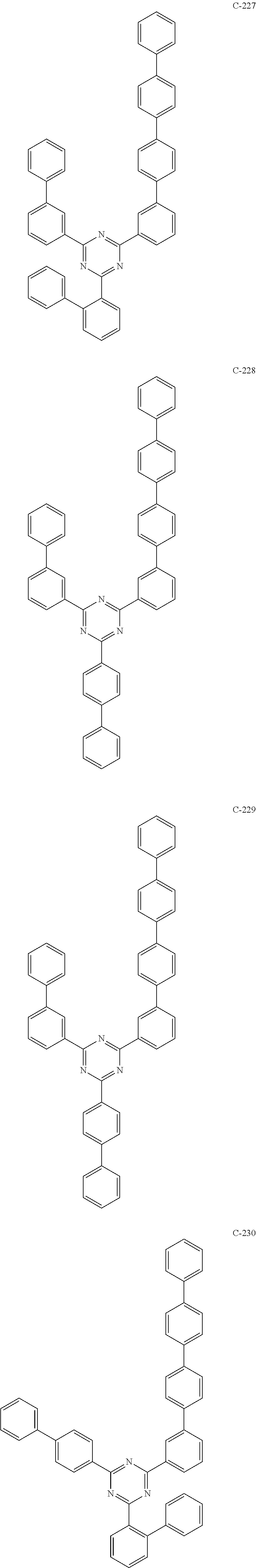 [SMILES: c1ccc(-c2ccc(-c3ccc(-c4cccc(-c5nc(-c6cccc(-c7ccccc7)c6)nc(-c6ccccc6-c6ccccc6)n5)c4)cc3)cc2)cc1 ; c1ccc(-c2ccc(-c3ccc(-c4cccc(-c5nc(-c6ccc(-c7ccccc7)cc6)nc(-c6cccc(-c7ccccc7)c6)n5)c4)cc3)cc2)cc1 ; c1ccc(-c2ccc(-c3ccc(-c4cccc(-c5nc(-c6ccc(-c7ccccc7)cc6)nc(-c6cccc(-c7ccccc7)c6)n5)c4)cc3)cc2)cc1 ; c1ccc(-c2ccc(-c3ccc(-c4cccc(-c5nc(-c6ccc(-c7ccccc7)cc6)nc(-c6ccccc6-c6ccccc6)n5)c4)cc3)cc2)cc1]

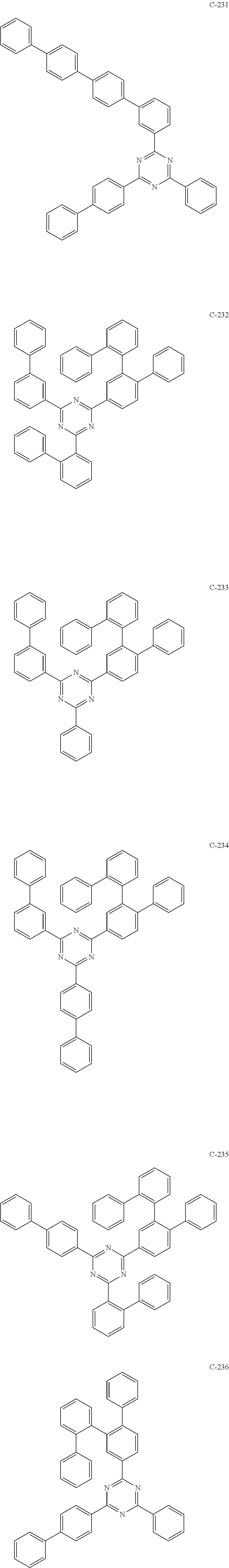

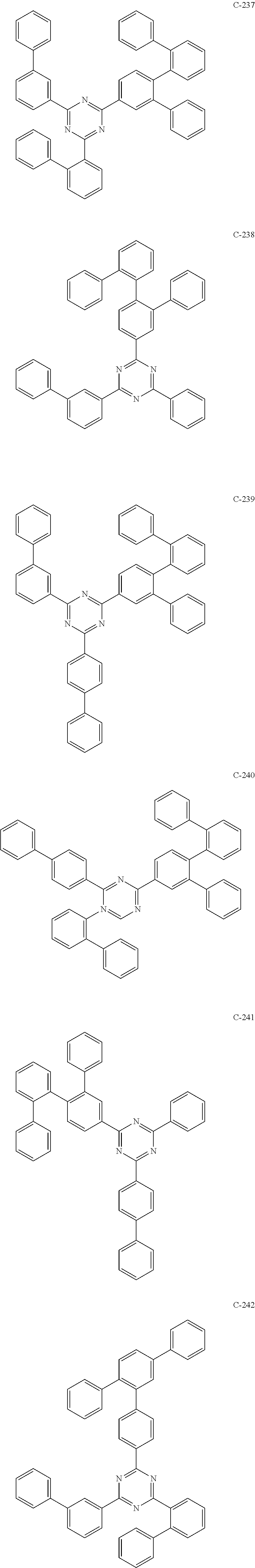

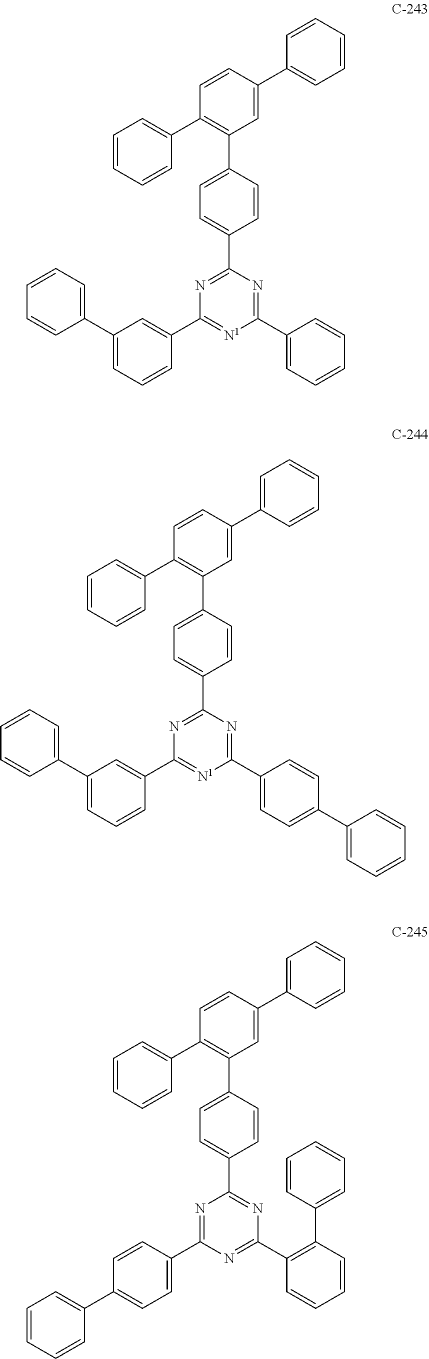

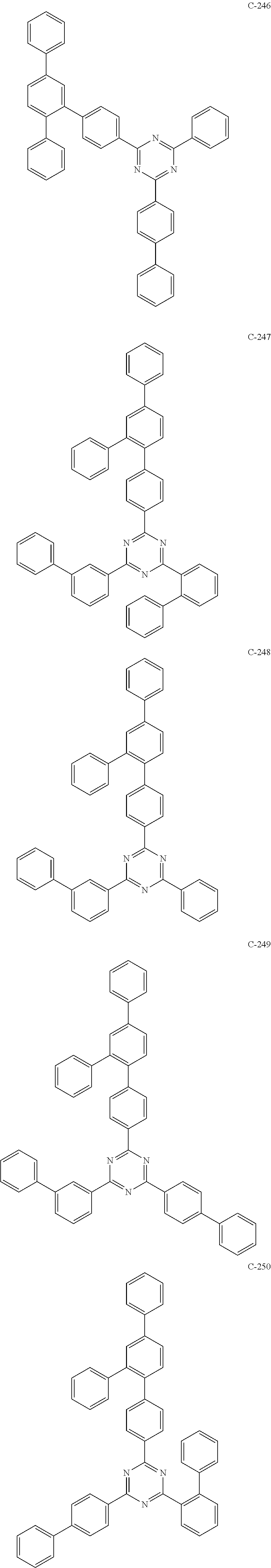

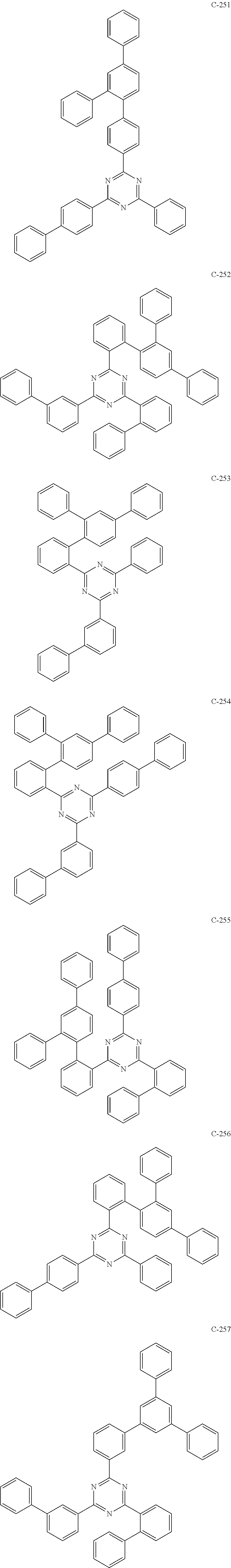

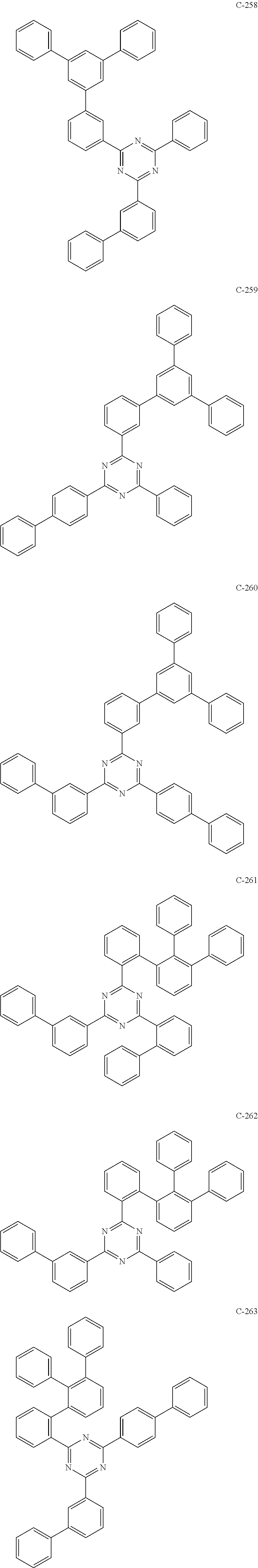

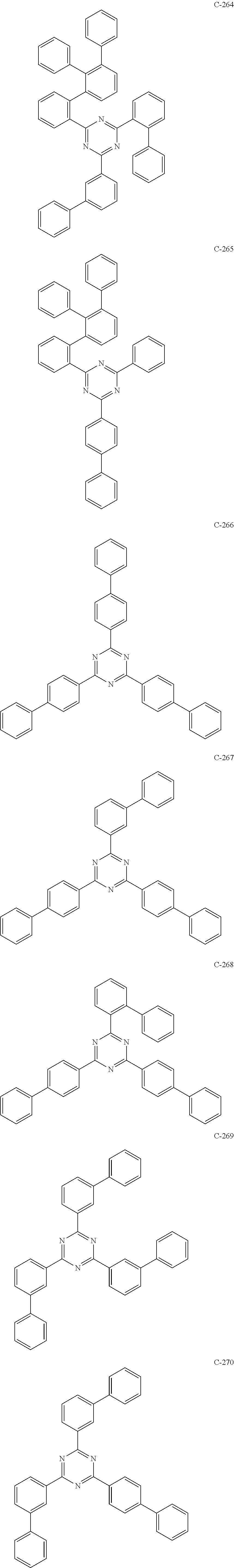

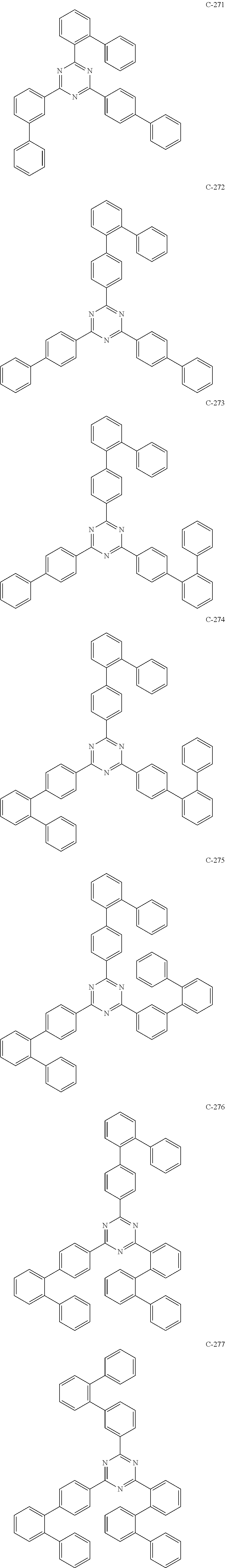

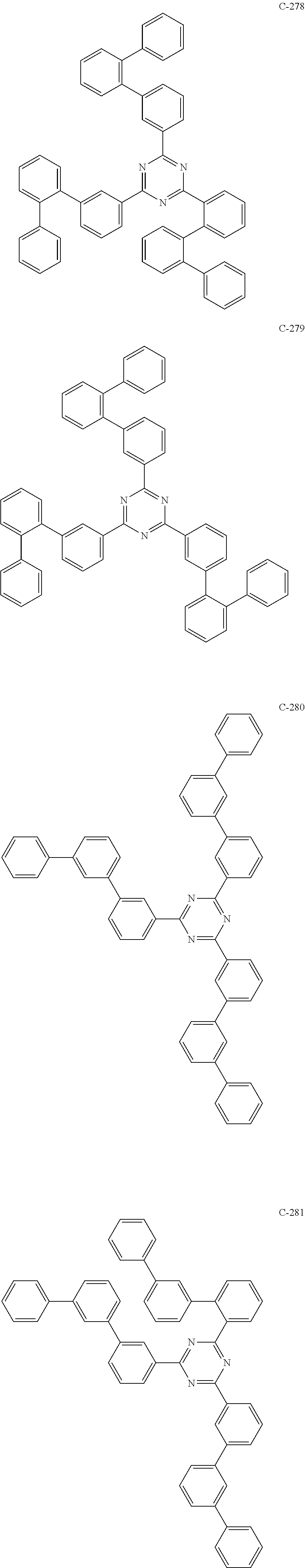

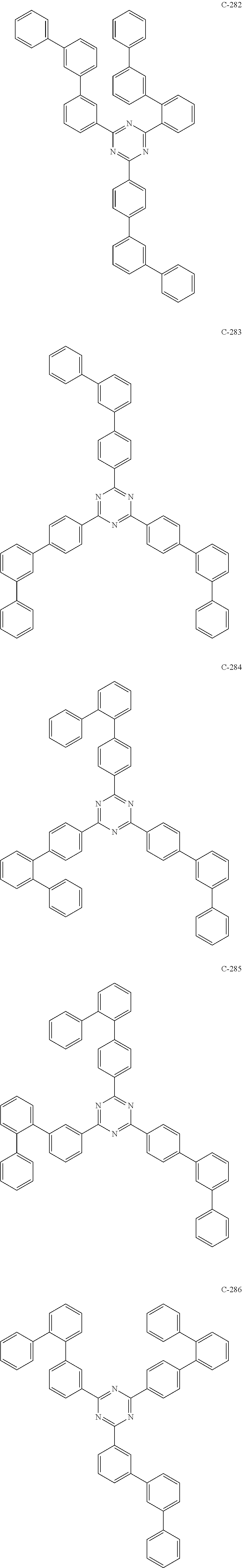

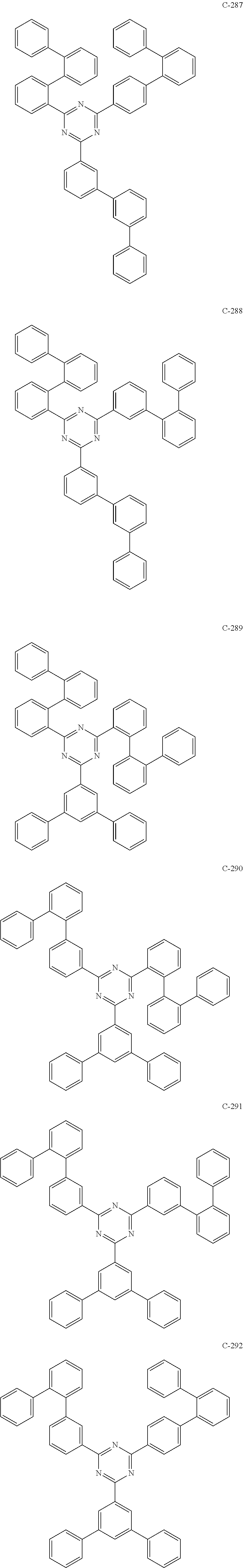

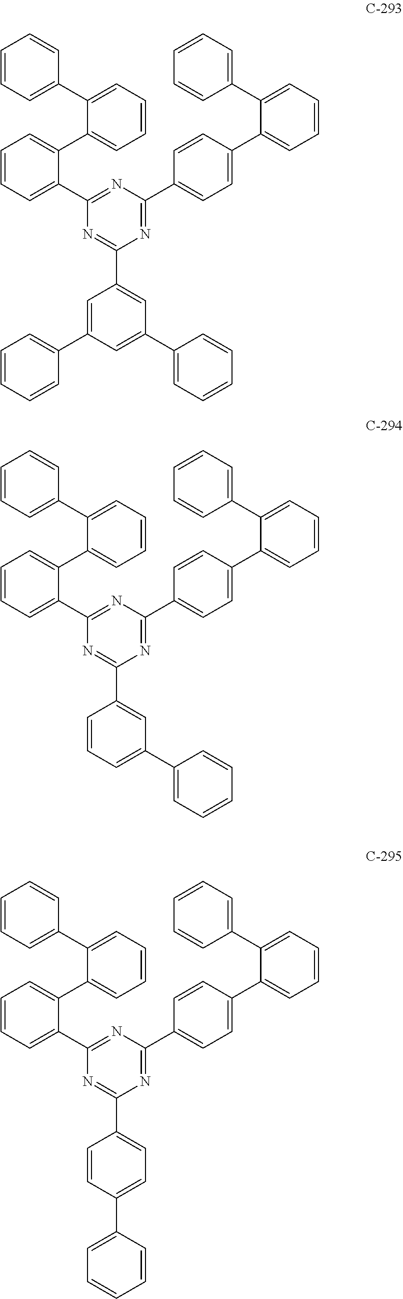 [SMILES: c1ccc(-c2cc(-c3ccccc3)cc(-c3nc(-c4ccc(-c5ccccc5-c5ccccc5)cc4)nc(-c4ccccc4-c4ccccc4-c4ccccc4)n3)c2)cc1 ; c1ccc(-c2cccc(-c3nc(-c4ccc(-c5ccccc5-c5ccccc5)cc4)nc(-c4ccccc4-c4ccccc4-c4ccccc4)n3)c2)cc1 ; c1ccc(-c2ccc(-c3nc(-c4ccc(-c5ccccc5-c5ccccc5)cc4)nc(-c4ccccc4-c4ccccc4-c4ccccc4)n3)cc2)cc1]

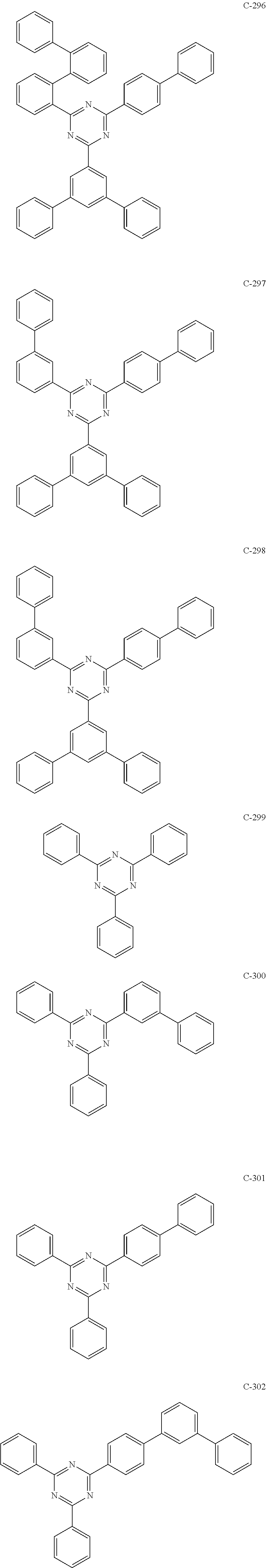

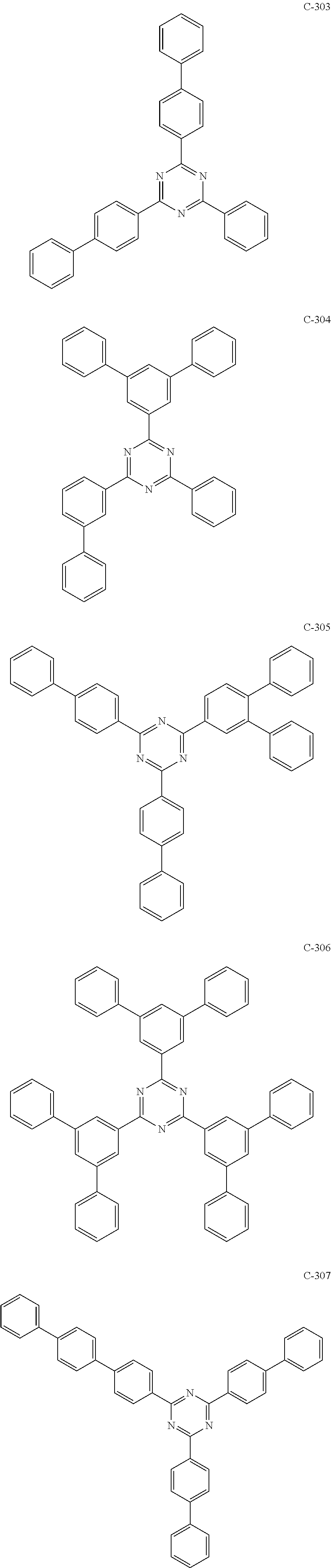 [SMILES: c1ccc(-c2ccc(-c3nc(-c4ccccc4)nc(-c4ccc(-c5ccccc5)cc4)n3)cc2)cc1 ; c1ccc(-c2cccc(-c3nc(-c4ccccc4)nc(-c4cc(-c5ccccc5)cc(-c5ccccc5)c4)n3)c2)cc1 ; c1ccc(-c2ccc(-c3nc(-c4ccc(-c5ccccc5)cc4)nc(-c4ccc(-c5ccccc5)c(-c5ccccc5)c4)n3)cc2)cc1 ; c1ccc(-c2cc(-c3ccccc3)cc(-c3nc(-c4cc(-c5ccccc5)cc(-c5ccccc5)c4)nc(-c4cc(-c5ccccc5)cc(-c5ccccc5)c4)n3)c2)cc1 ; c1ccc(-c2ccc(-c3ccc(-c4nc(-c5ccc(-c6ccccc6)cc5)nc(-c5ccc(-c6ccccc6)cc5)n4)cc3)cc2)cc1]

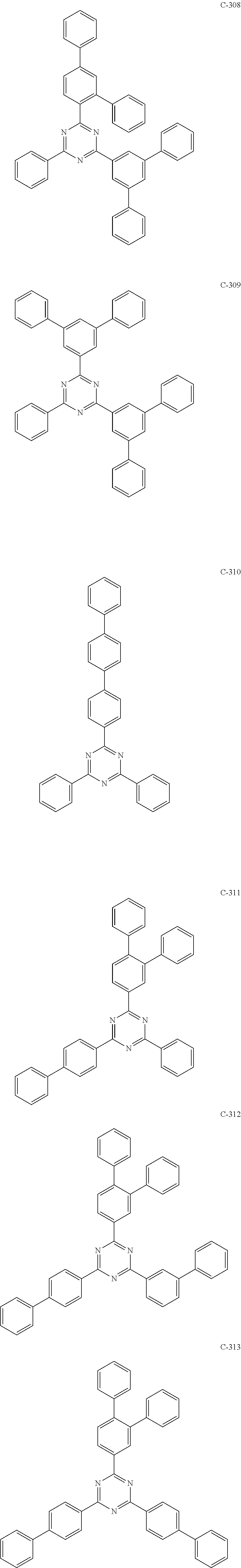

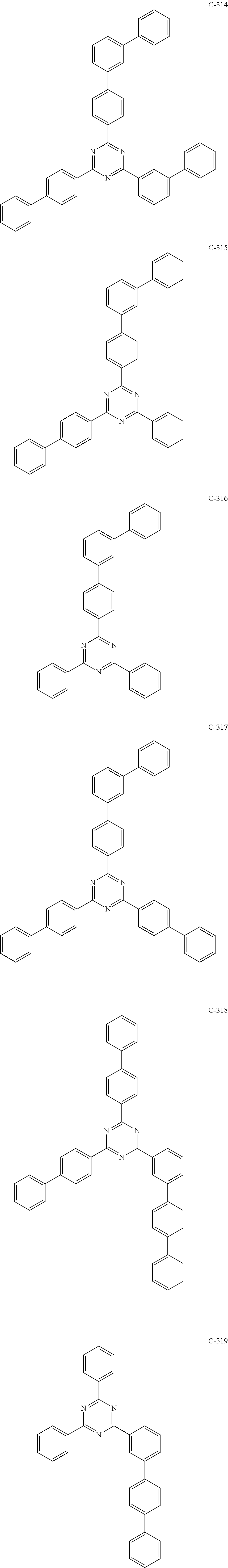 [SMILES: c1ccc(-c2ccc(-c3nc(-c4ccc(-c5cccc(-c6ccccc6)c5)cc4)nc(-c4cccc(-c5ccccc5)c4)n3)cc2)cc1 ; c1ccc(-c2ccc(-c3nc(-c4ccccc4)nc(-c4ccc(-c5cccc(-c6ccccc6)c5)cc4)n3)cc2)cc1 ; c1ccc(-c2cccc(-c3ccc(-c4nc(-c5ccccc5)nc(-c5ccccc5)n4)cc3)c2)cc1 ; c1ccc(-c2ccc(-c3nc(-c4ccc(-c5ccccc5)cc4)nc(-c4ccc(-c5cccc(-c6ccccc6)c5)cc4)n3)cc2)cc1 ; c1ccc(-c2ccc(-c3cccc(-c4nc(-c5ccc(-c6ccccc6)cc5)nc(-c5ccc(-c6ccccc6)cc5)n4)c3)cc2)cc1 ; c1ccc(-c2ccc(-c3cccc(-c4nc(-c5ccccc5)nc(-c5ccccc5)n4)c3)cc2)cc1]

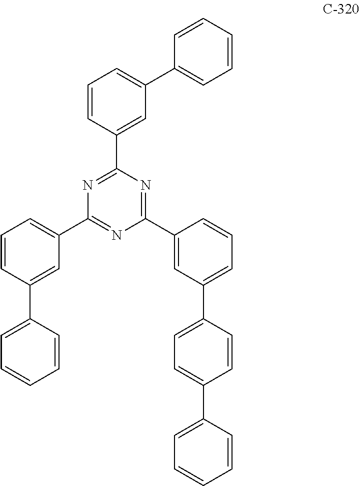 [SMILES: c1ccc(-c2ccc(-c3cccc(-c4nc(-c5cccc(-c6ccccc6)c5)nc(-c5cccc(-c6ccccc6)c5)n4)c3)cc2)cc1]

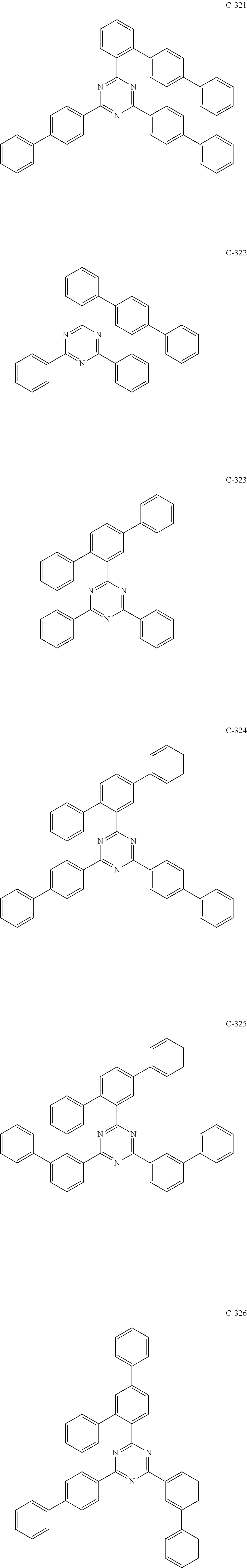 [SMILES: c1ccc(-c2ccc(-c3nc(-c4ccc(-c5ccccc5)cc4)nc(-c4ccccc4-c4ccc(-c5ccccc5)cc4)n3)cc2)cc1 ; c1ccc(-c2ccc(-c3ccccc3-c3nc(-c4ccccc4)nc(-c4ccccc4)n3)cc2)cc1 ; c1ccc(-c2ccc(-c3ccccc3)c(-c3nc(-c4ccccc4)nc(-c4ccccc4)n3)c2)cc1 ; c1ccc(-c2ccc(-c3nc(-c4ccc(-c5ccccc5)cc4)nc(-c4cc(-c5ccccc5)ccc4-c4ccccc4)n3)cc2)cc1 ; c1ccc(-c2cccc(-c3nc(-c4cccc(-c5ccccc5)c4)nc(-c4cc(-c5ccccc5)ccc4-c4ccccc4)n3)c2)cc1 ; c1ccc(-c2ccc(-c3nc(-c4cccc(-c5ccccc5)c4)nc(-c4ccc(-c5ccccc5)cc4-c4ccccc4)n3)cc2)cc1]

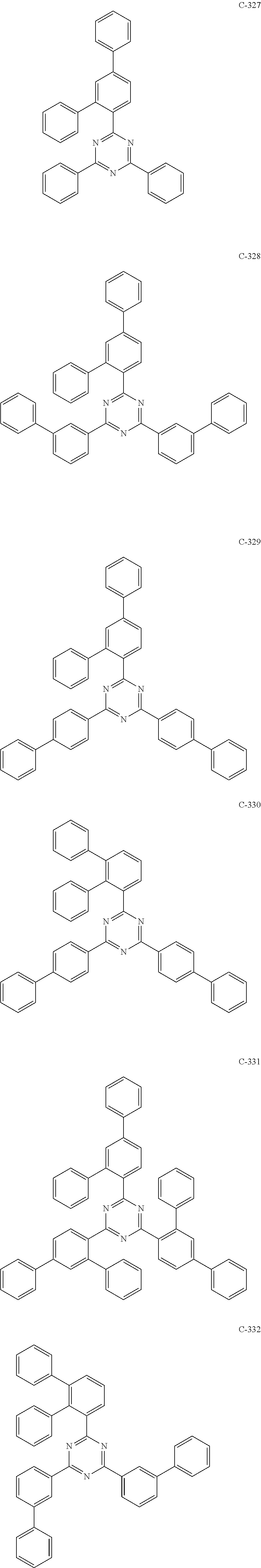

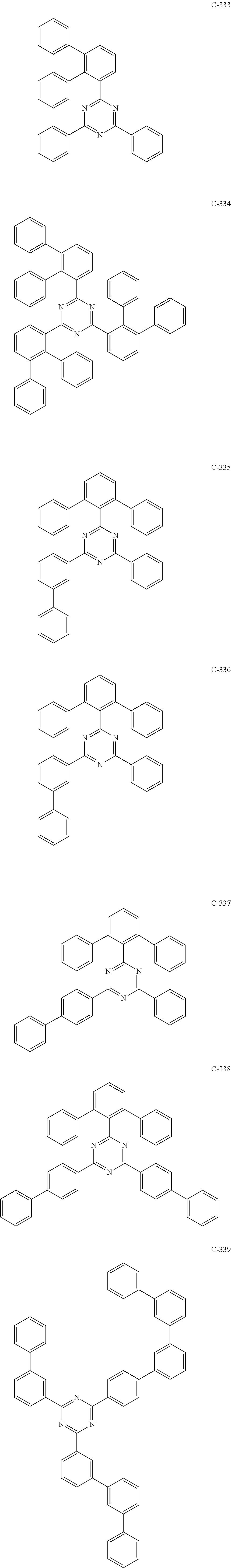

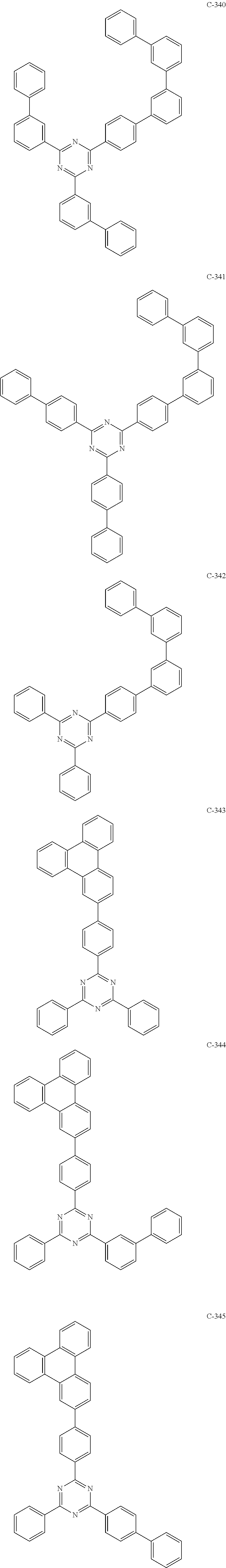

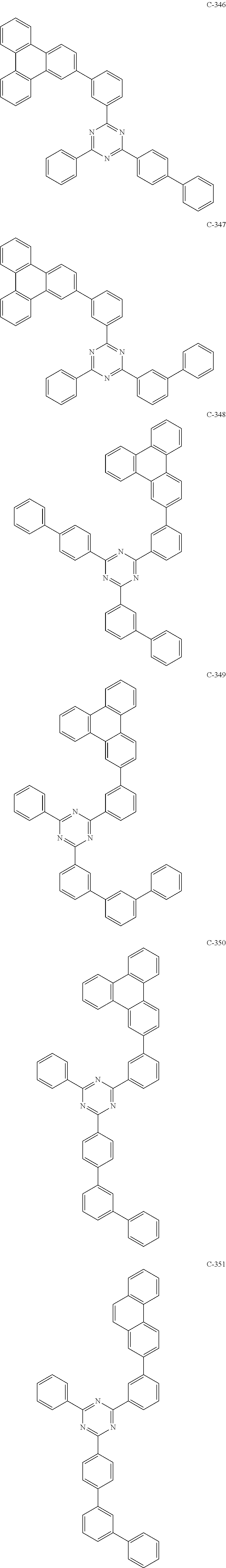

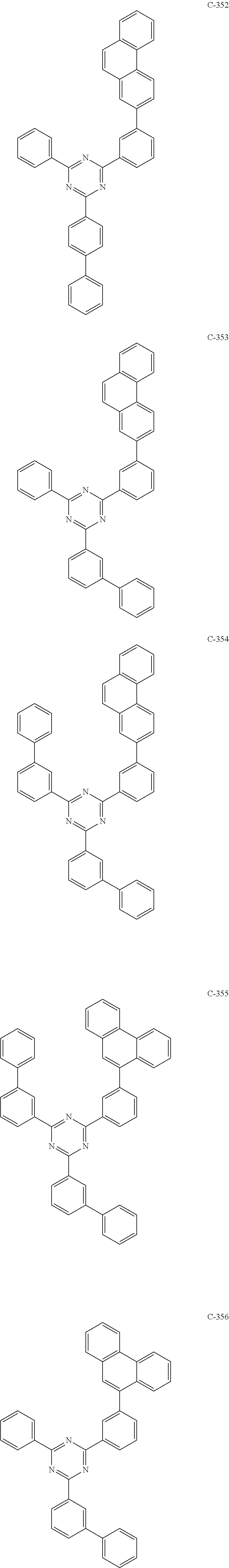

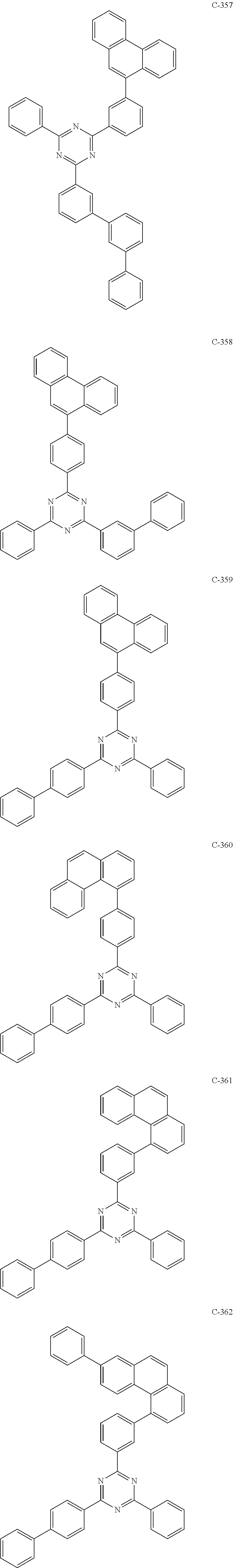 [SMILES: c1ccc(-c2cccc(-c3cccc(-c4nc(-c5ccccc5)nc(-c5cccc(-c6cc7ccccc7c7ccccc67)c5)n4)c3)c2)cc1 ; c1ccc(-c2cccc(-c3nc(-c4ccccc4)nc(-c4ccc(-c5cc6ccccc6c6ccccc56)cc4)n3)c2)cc1 ; c1ccc(-c2ccc(-c3nc(-c4ccccc4)nc(-c4ccc(-c5cc6ccccc6c6ccccc56)cc4)n3)cc2)cc1 ; c1ccc(-c2ccc(-c3nc(-c4ccccc4)nc(-c4ccc(-c5cccc6ccc7ccccc7c56)cc4)n3)cc2)cc1 ; c1ccc(-c2ccc(-c3nc(-c4ccccc4)nc(-c4cccc(-c5cccc6ccc7ccccc7c56)c4)n3)cc2)cc1 ; c1ccc(-c2ccc(-c3nc(-c4ccccc4)nc(-c4cccc(-c5cccc6ccc7cc(-c8ccccc8)ccc7c56)c4)n3)cc2)cc1]

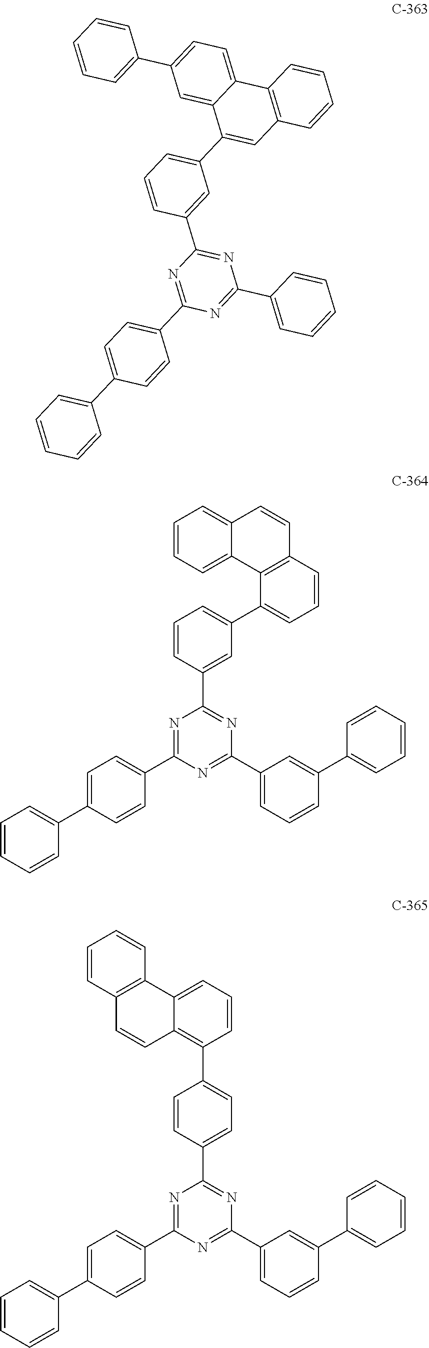 [SMILES: c1ccc(-c2ccc(-c3nc(-c4ccccc4)nc(-c4cccc(-c5cc6ccccc6c6ccc(-c7ccccc7)cc56)c4)n3)cc2)cc1 ; c1ccc(-c2ccc(-c3nc(-c4cccc(-c5ccccc5)c4)nc(-c4cccc(-c5cccc6ccc7ccccc7c56)c4)n3)cc2)cc1 ; c1ccc(-c2ccc(-c3nc(-c4ccc(-c5cccc6c5ccc5ccccc56)cc4)nc(-c4cccc(-c5ccccc5)c4)n3)cc2)cc1]

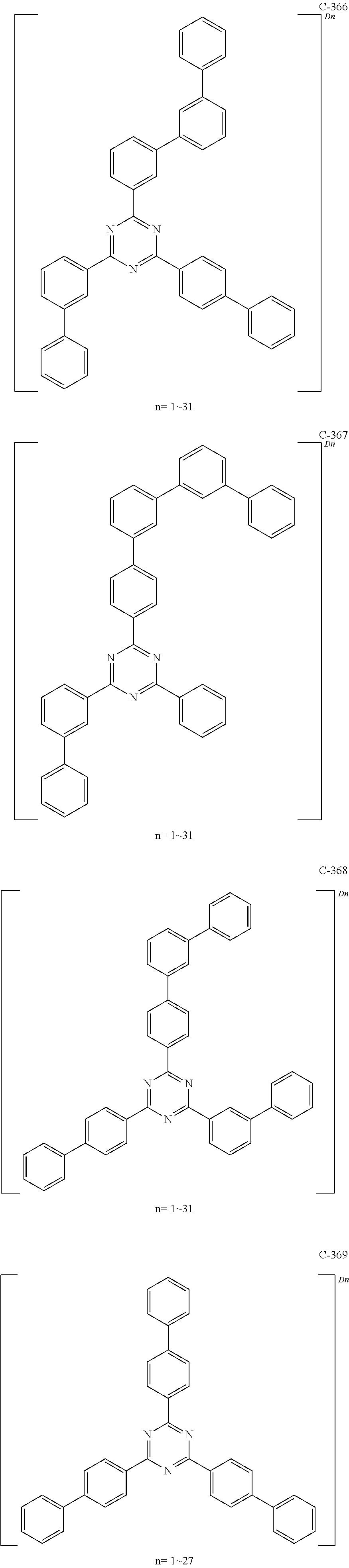

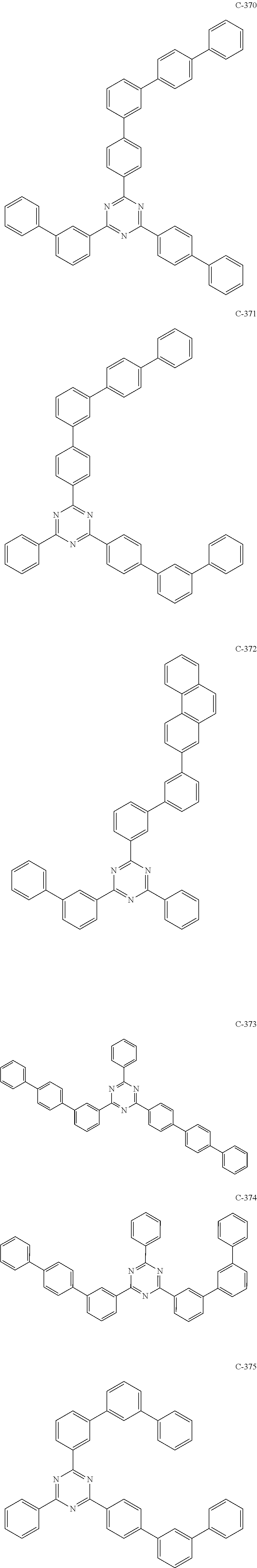

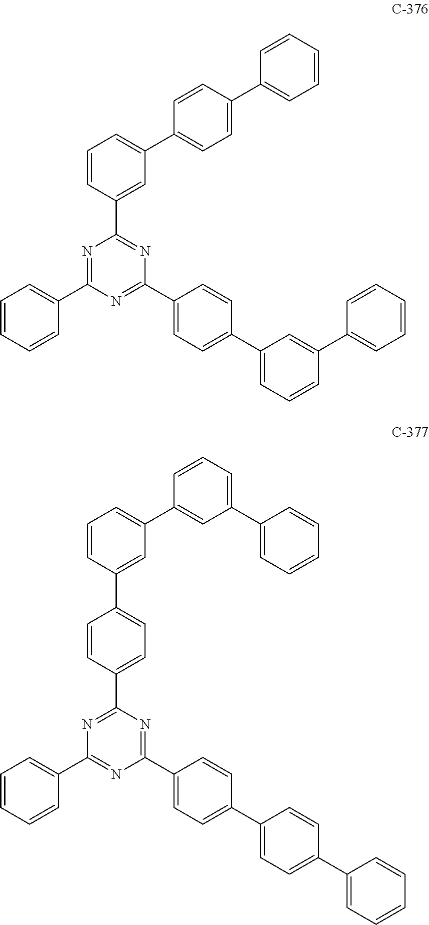 [SMILES: c1ccc(-c2ccc(-c3cccc(-c4nc(-c5ccccc5)nc(-c5ccc(-c6cccc(-c7ccccc7)c6)cc5)n4)c3)cc2)cc1 ; c1ccc(-c2ccc(-c3ccc(-c4nc(-c5ccccc5)nc(-c5ccc(-c6cccc(-c7cccc(-c8ccccc8)c7)c6)cc5)n4)cc3)cc2)cc1]

In the above compounds, Dn means that n number of hydrogen atoms are replaced with deuterium.

The compound represented by formula 2 may be at least one selected from the following compounds, but is not limited thereto.

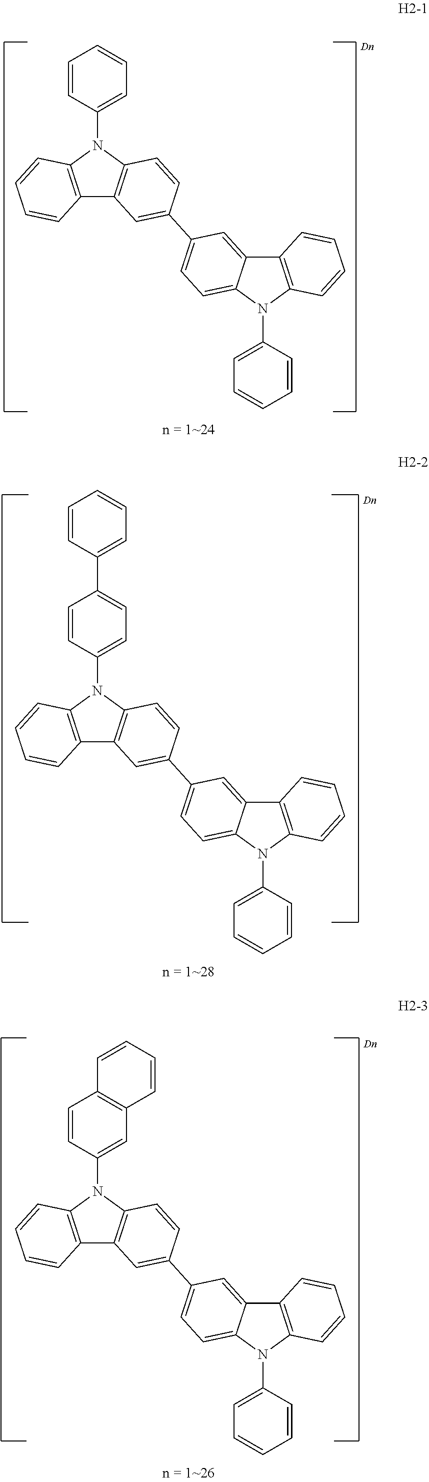

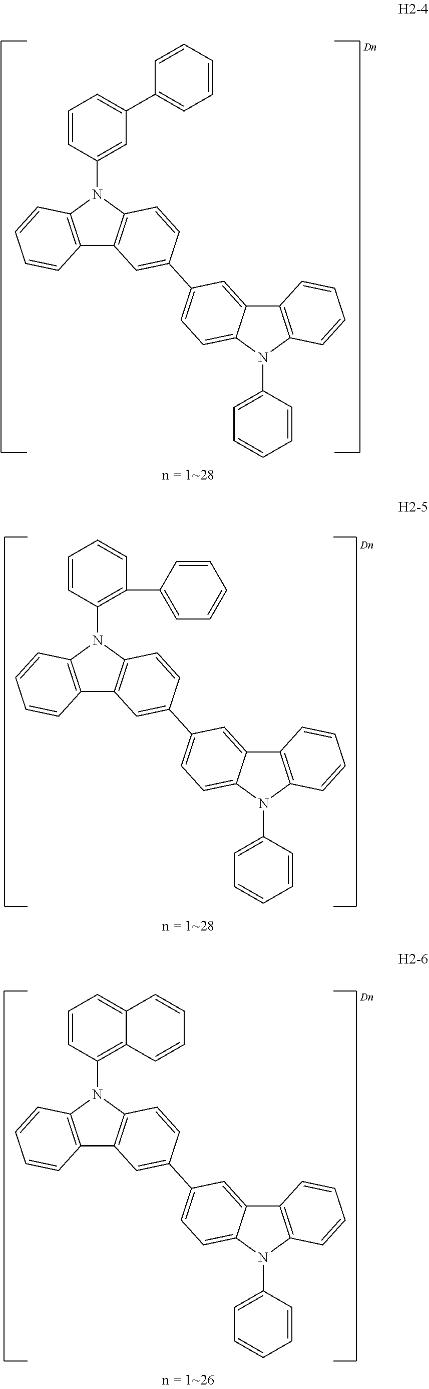

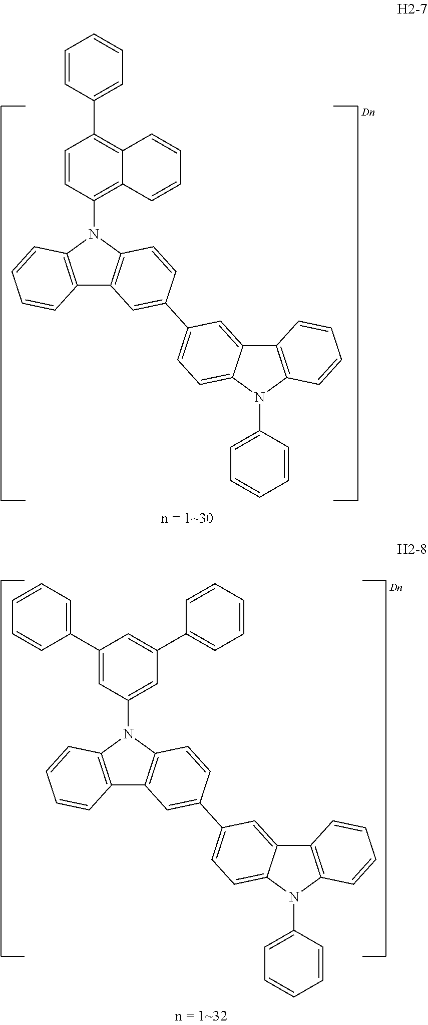

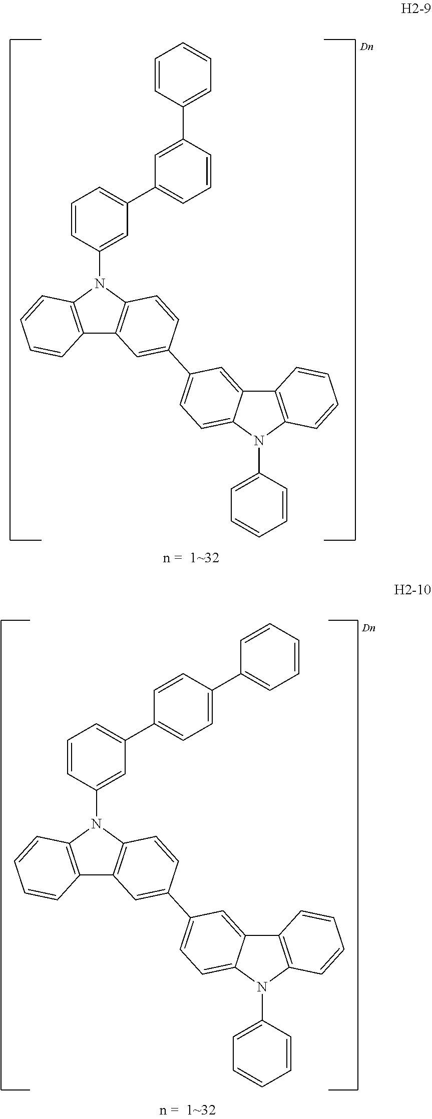

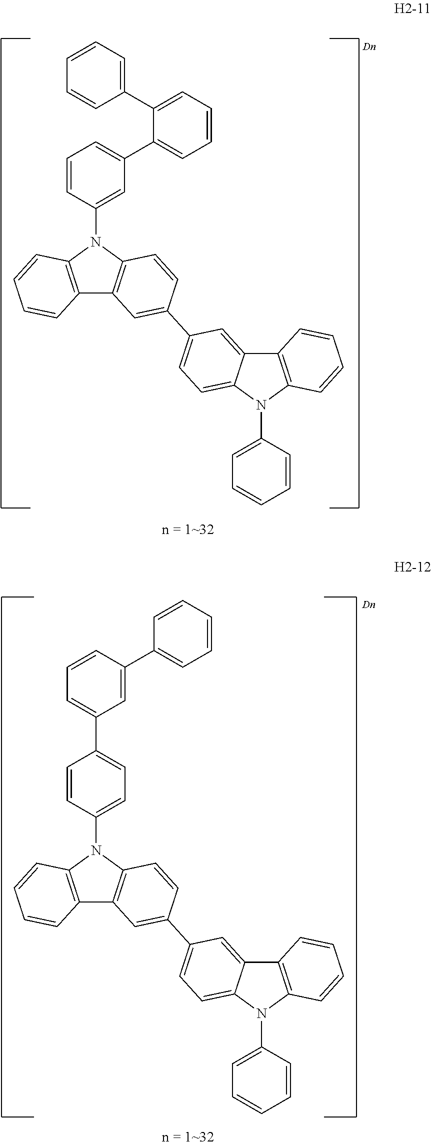

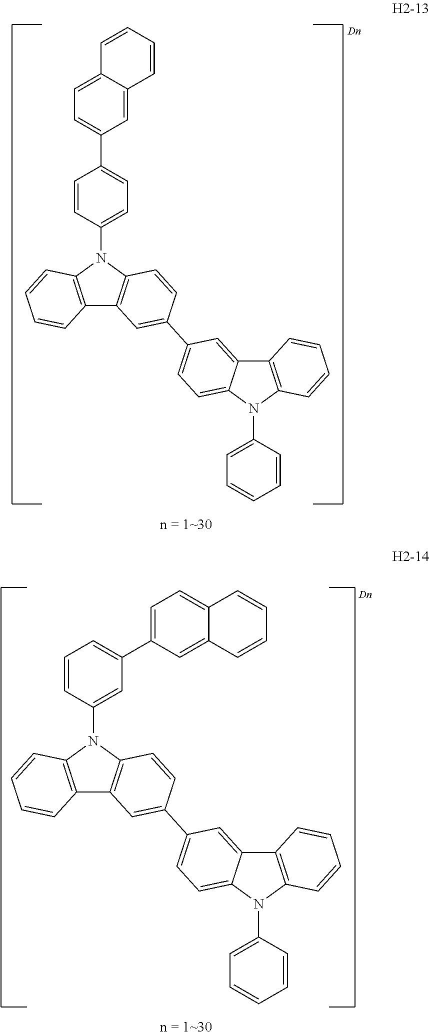

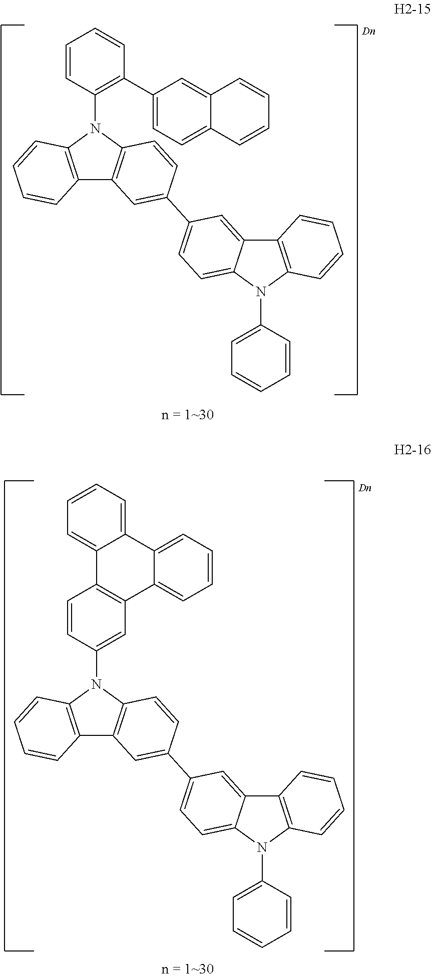

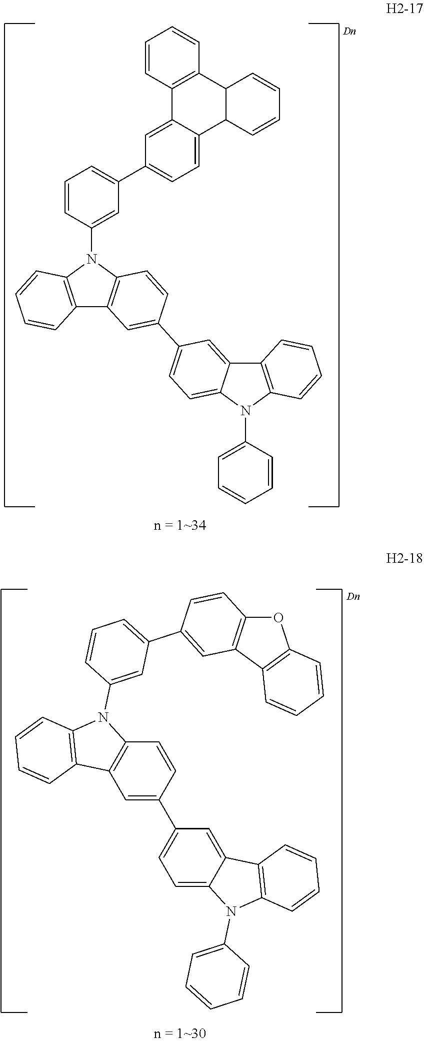

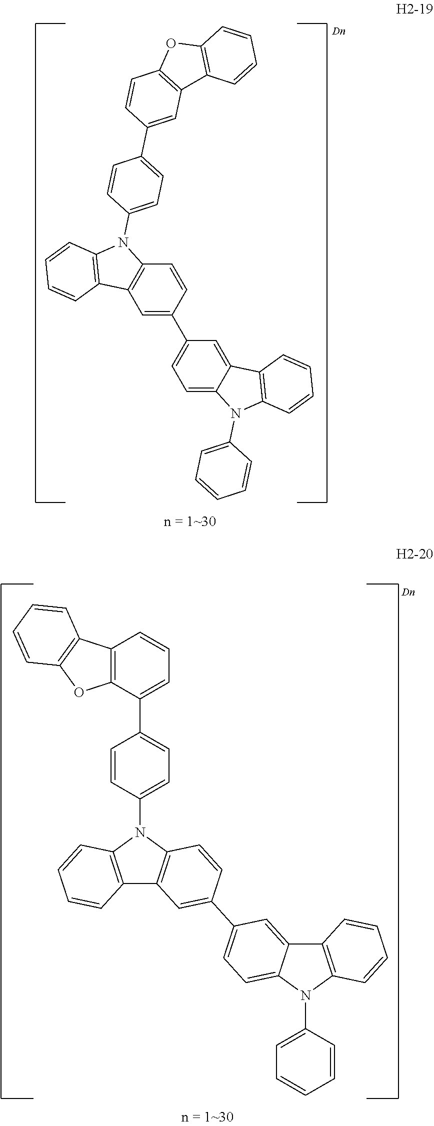

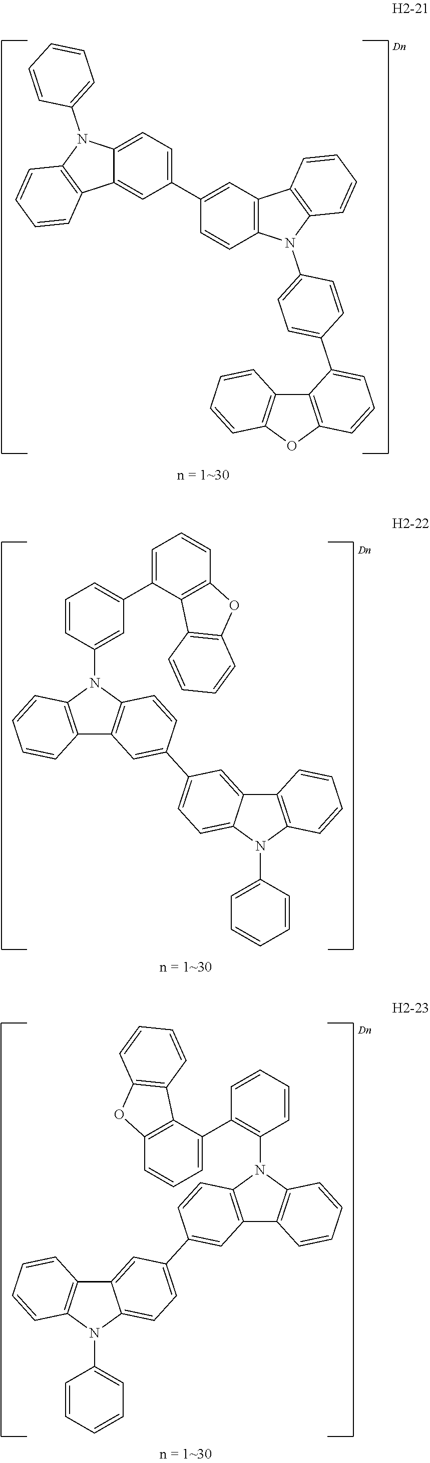

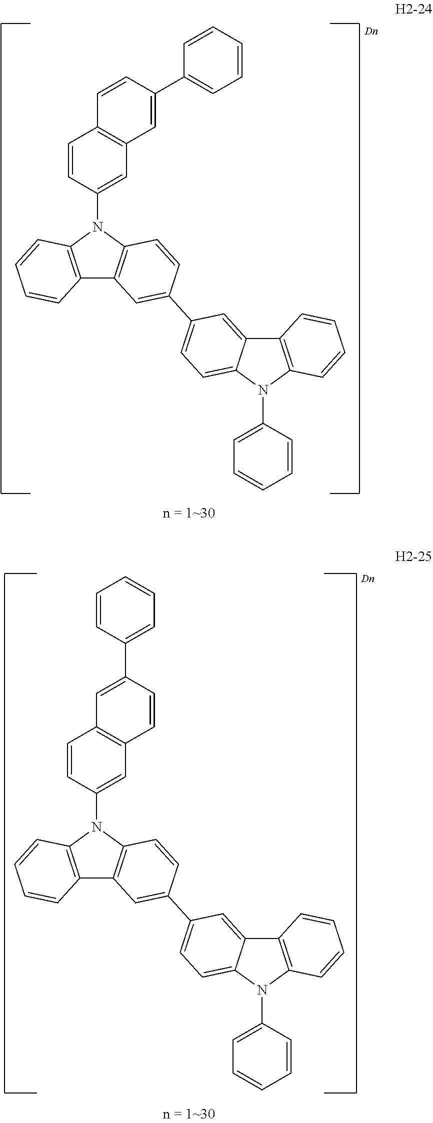

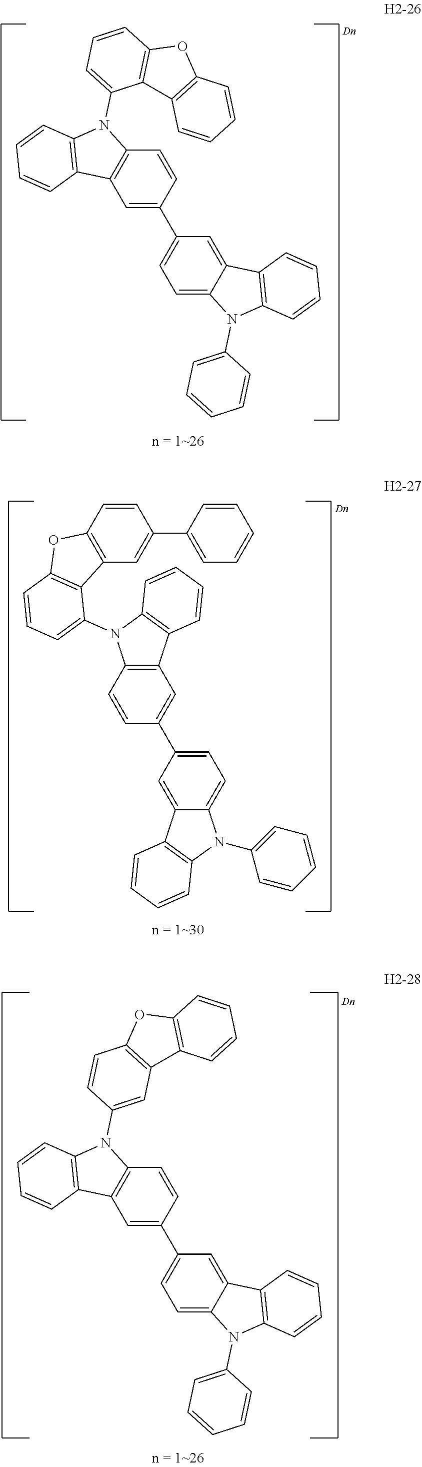

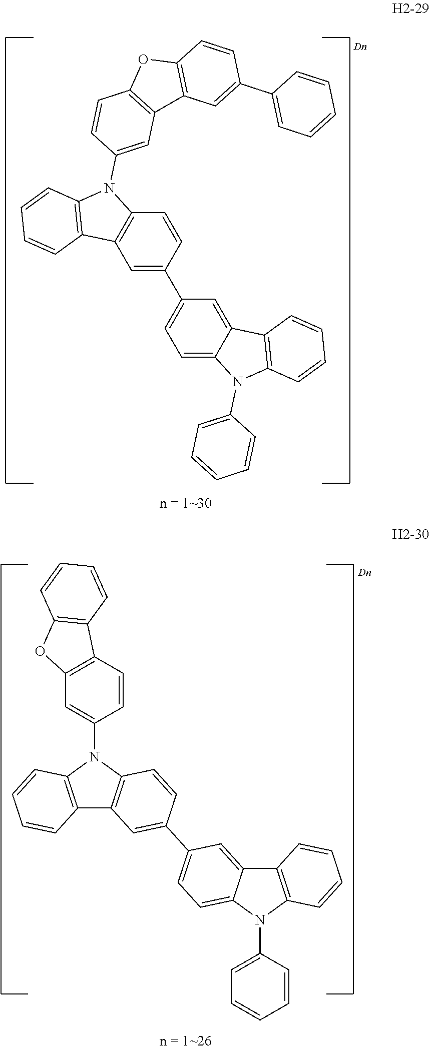

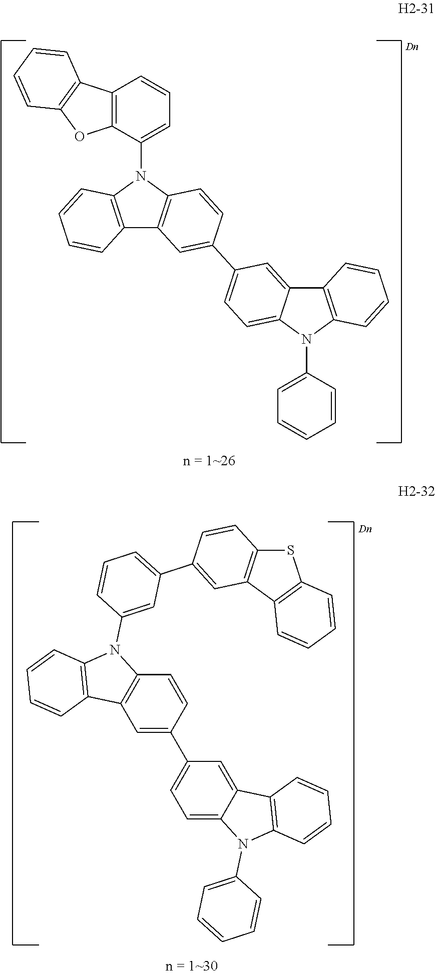

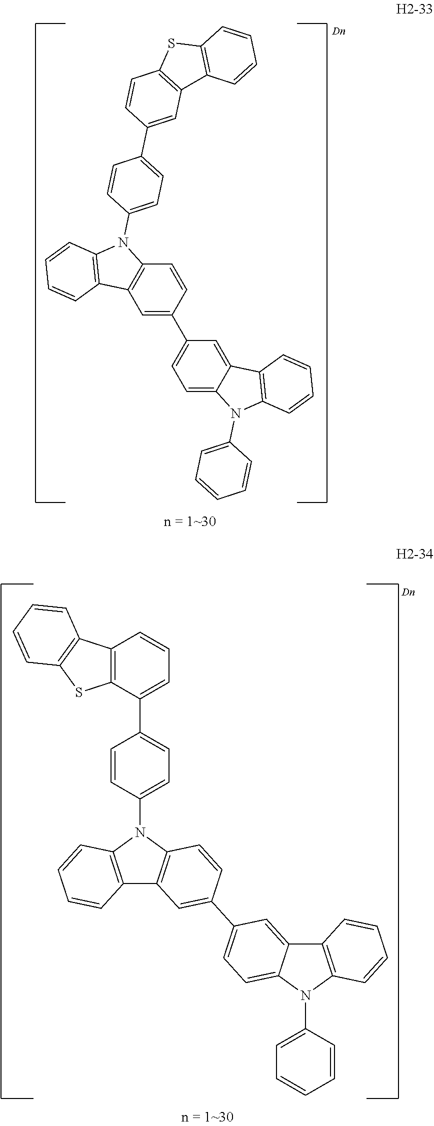

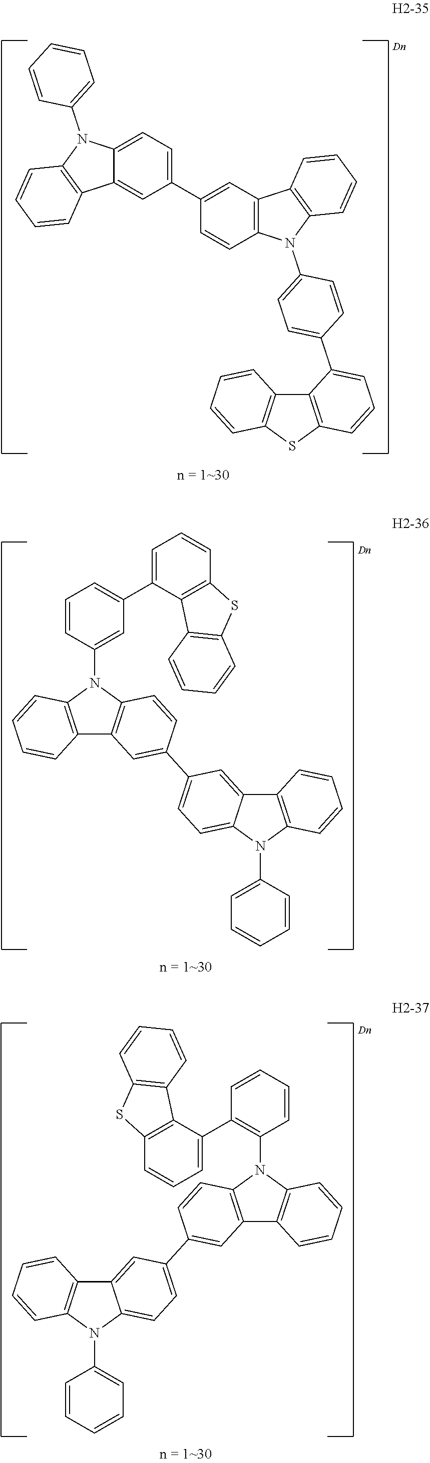

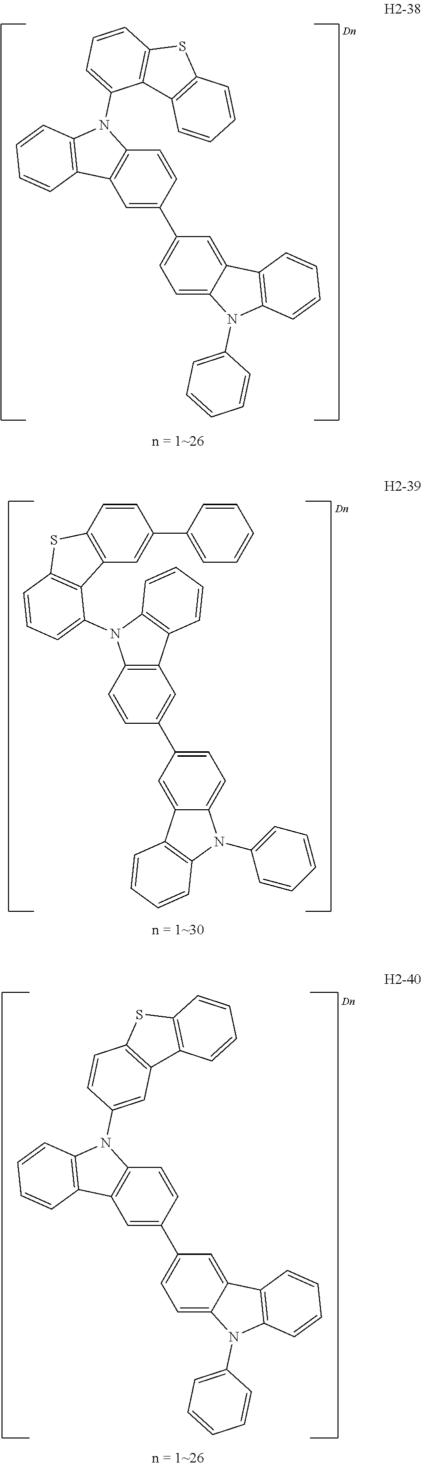

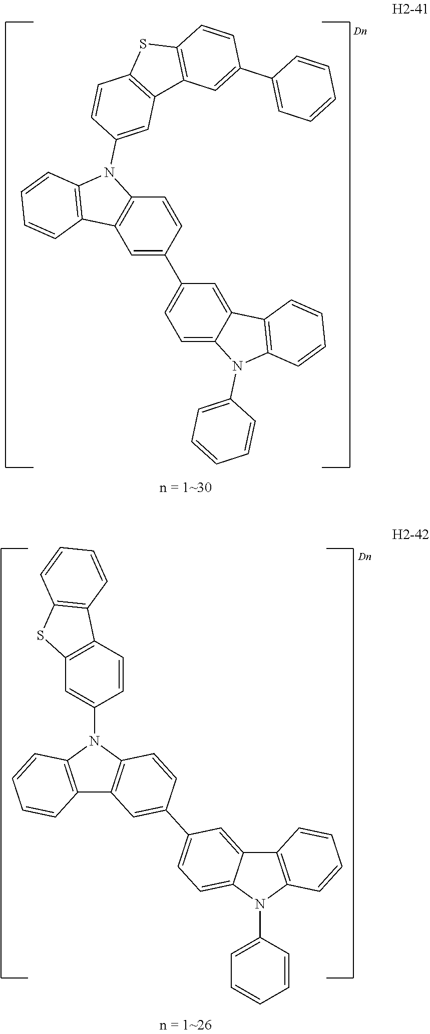

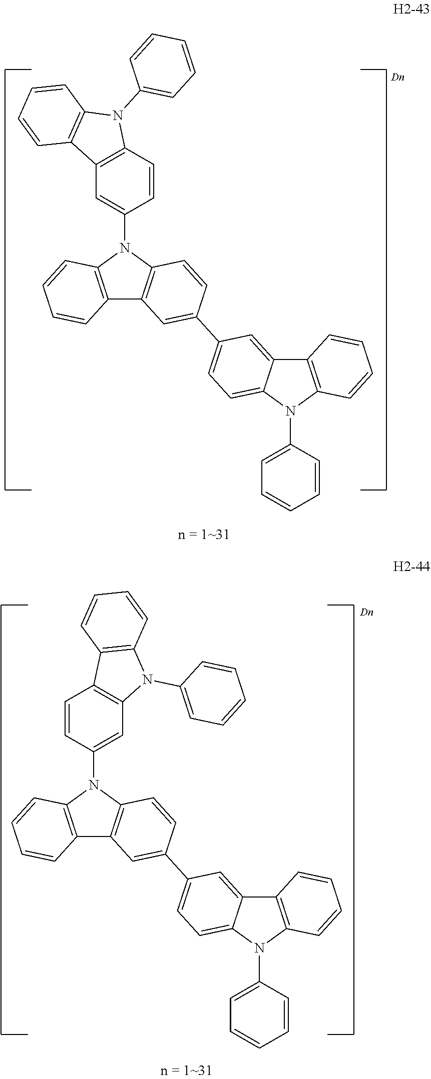

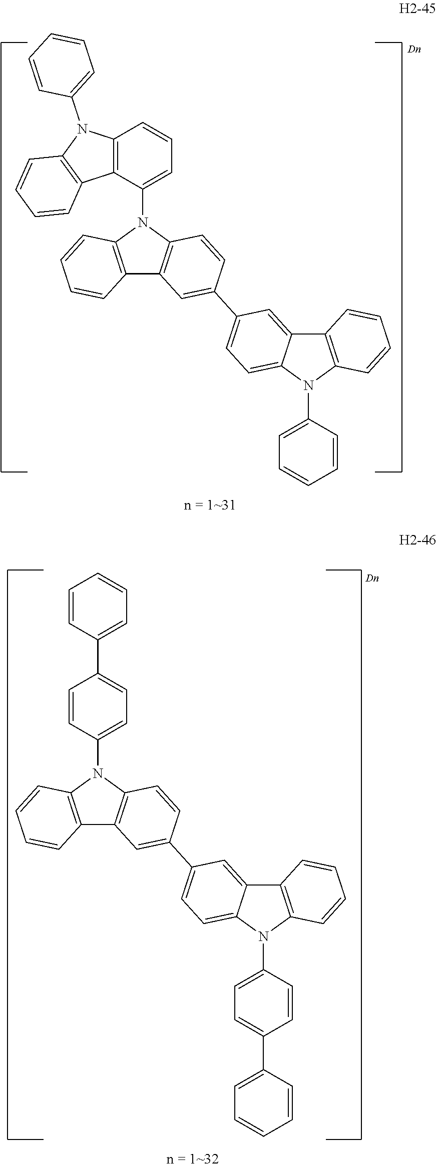

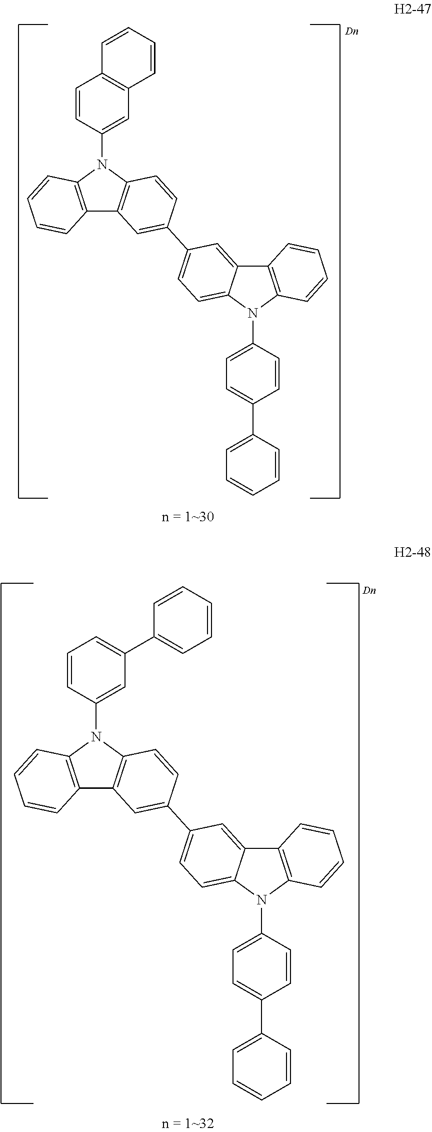

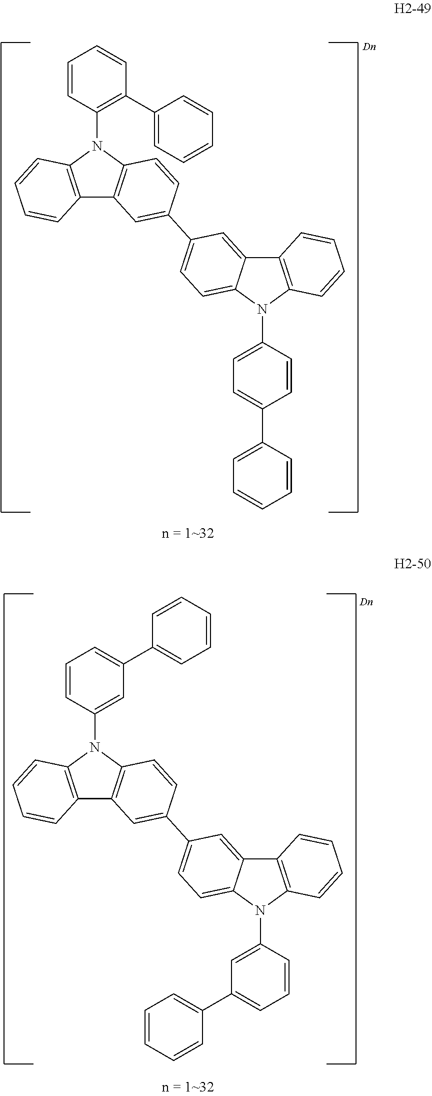

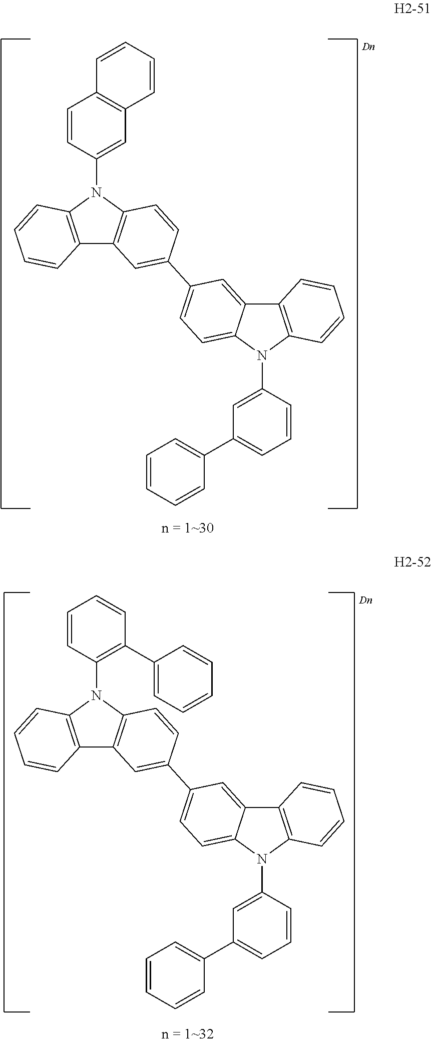

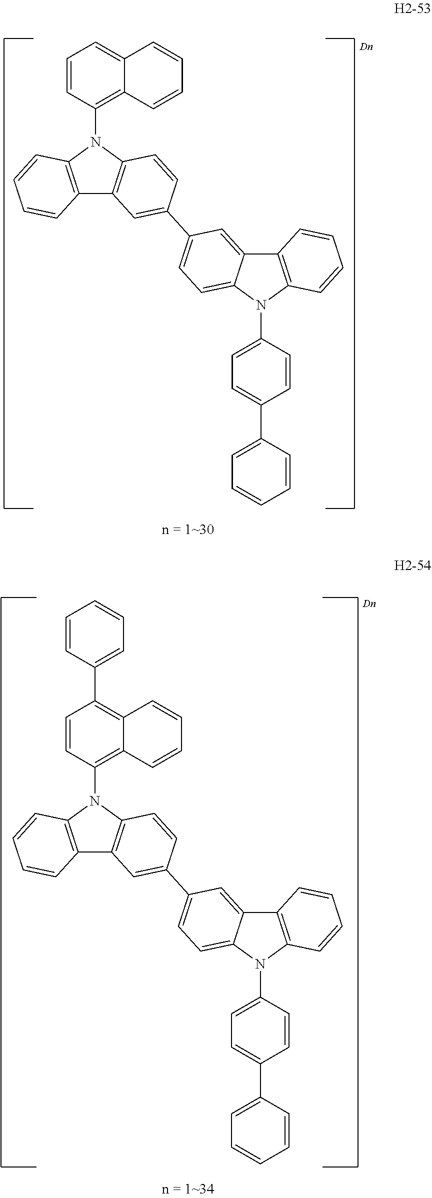

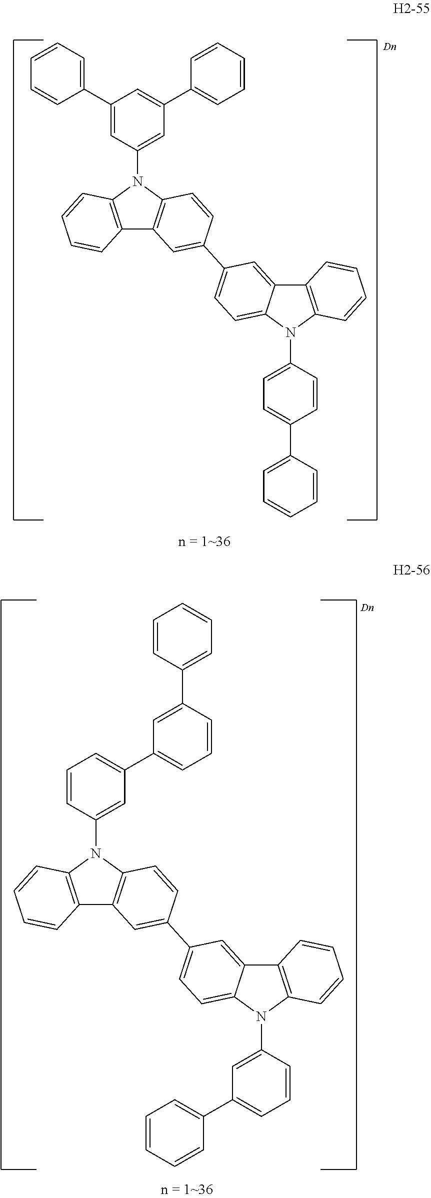

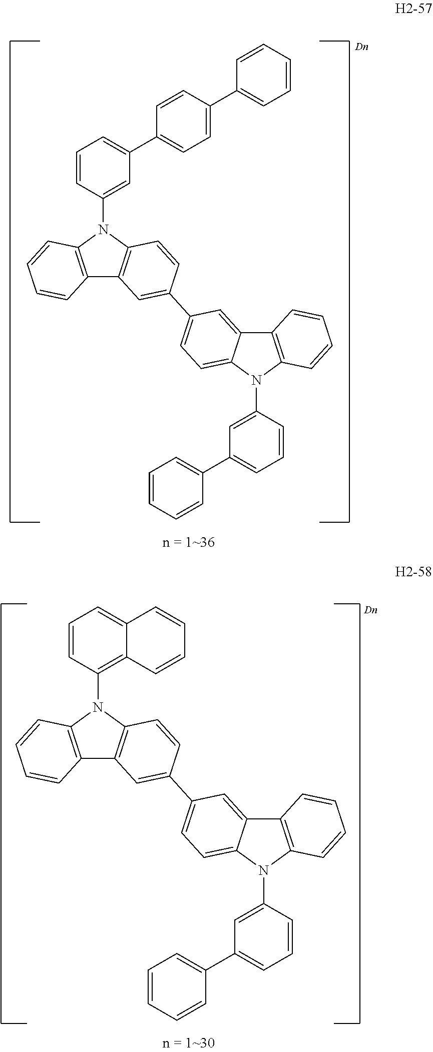

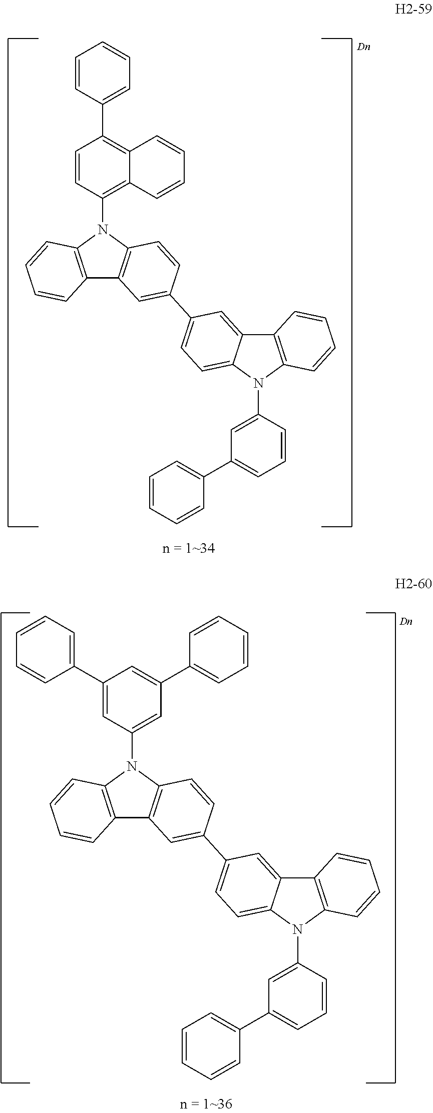

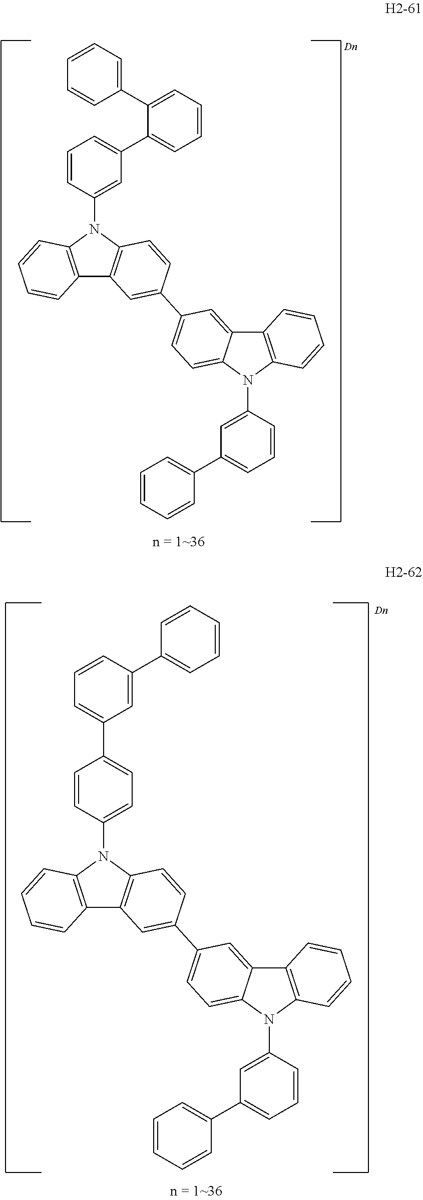

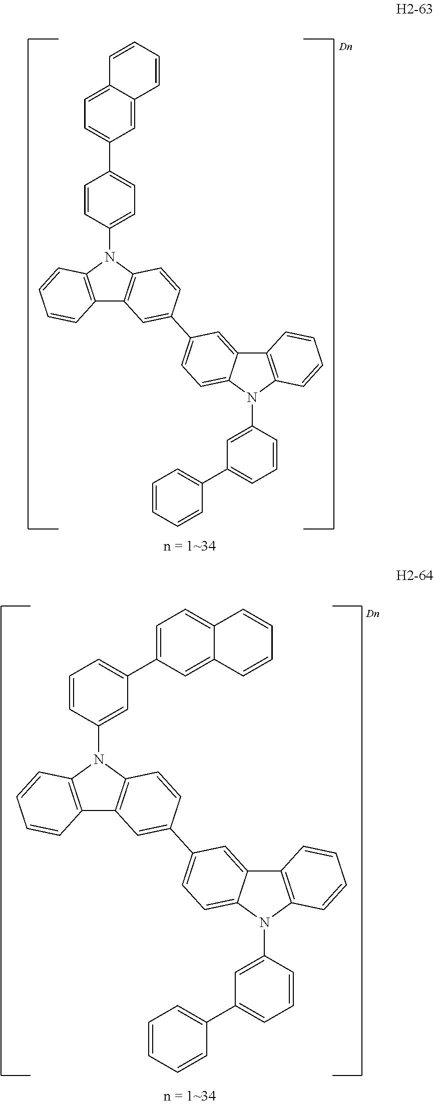

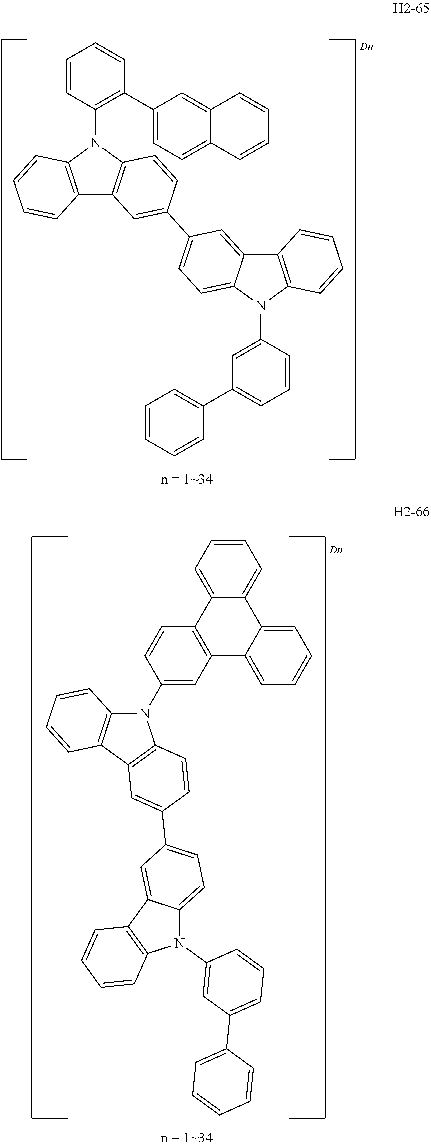

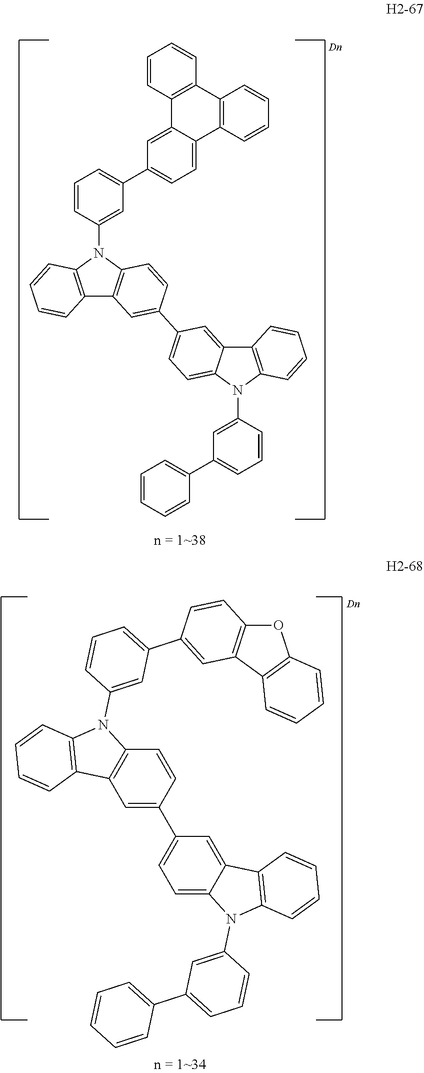

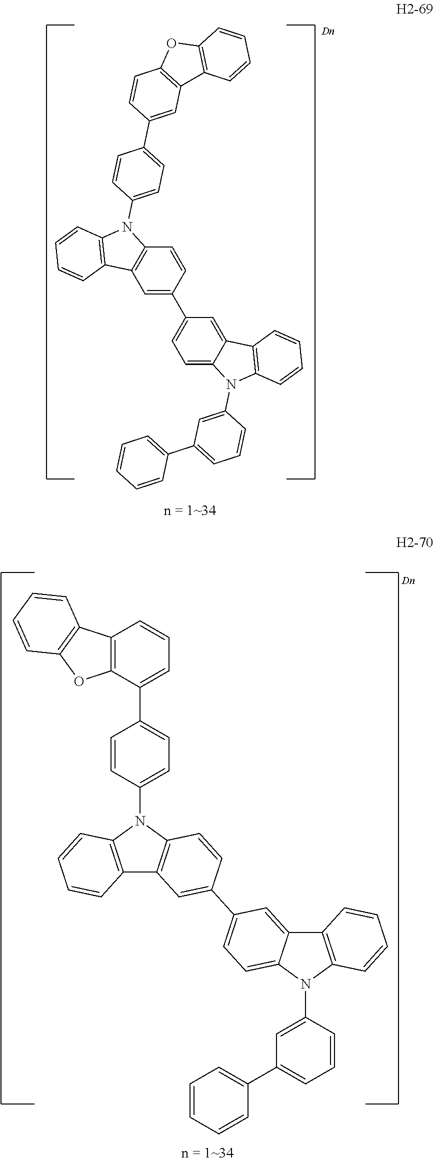

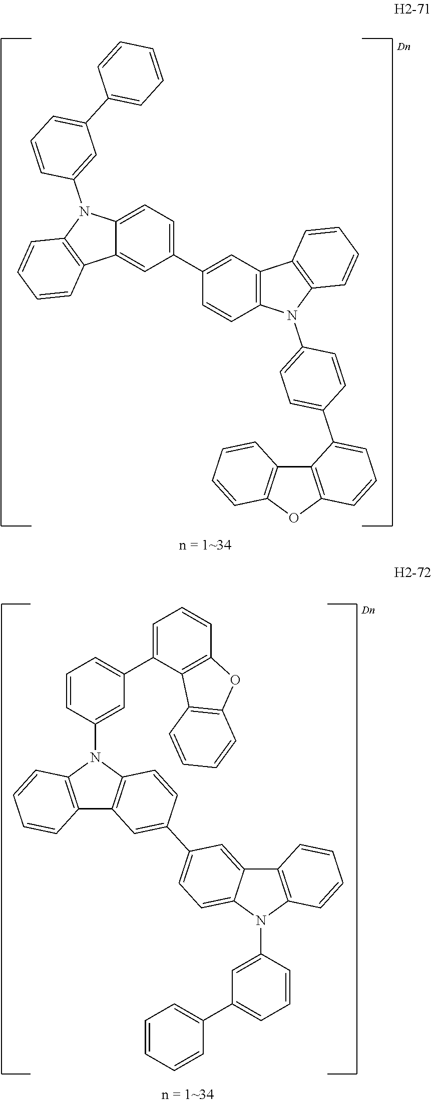

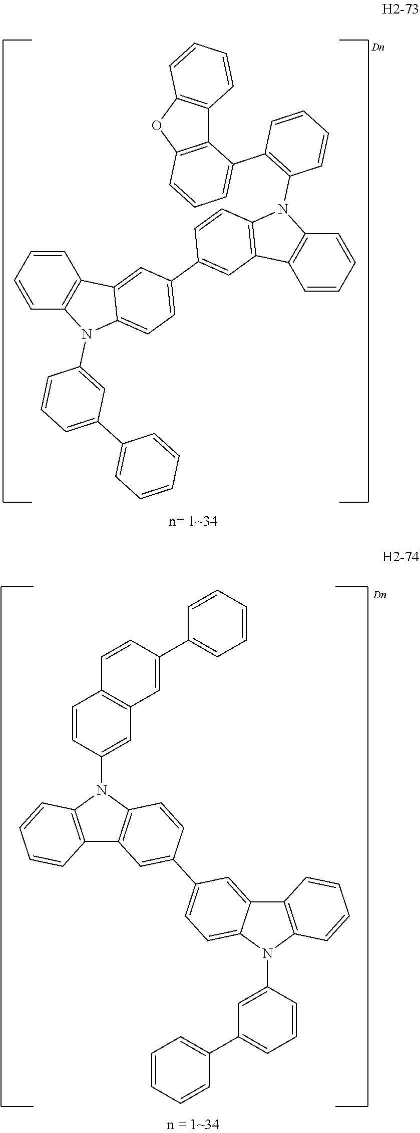

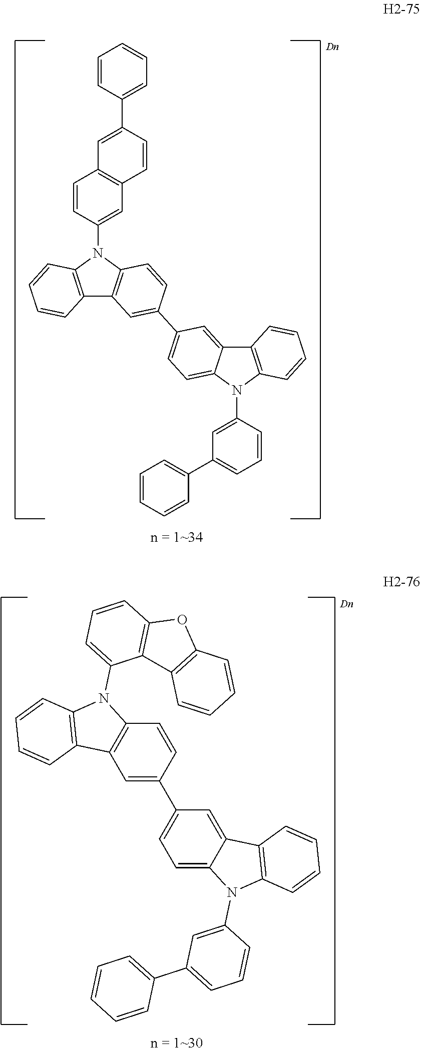

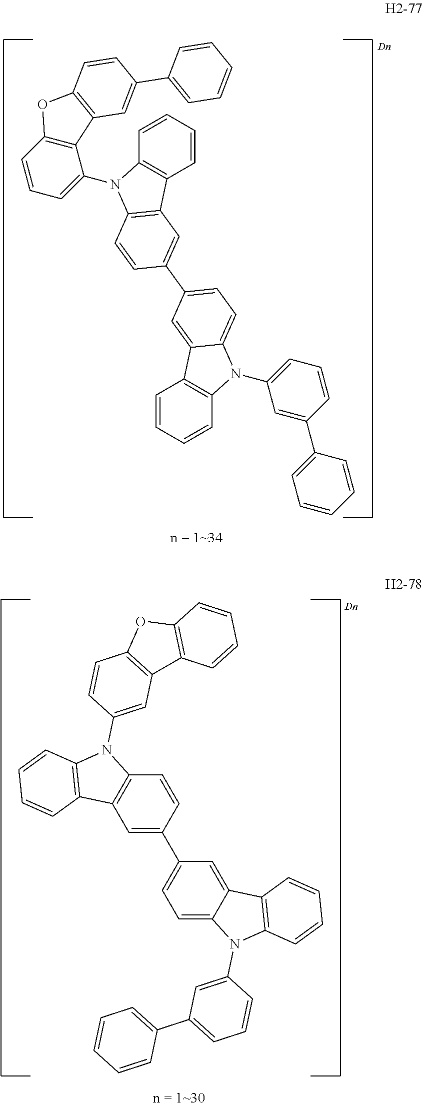

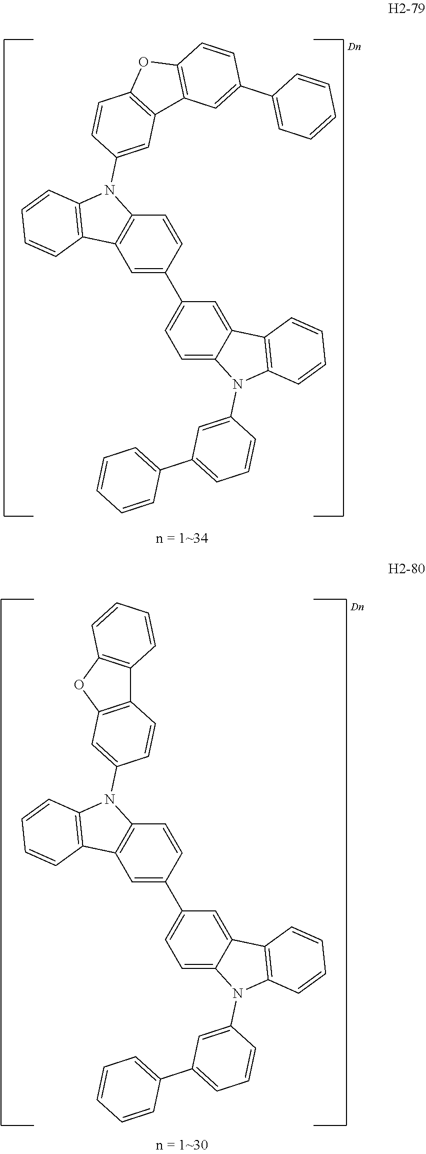

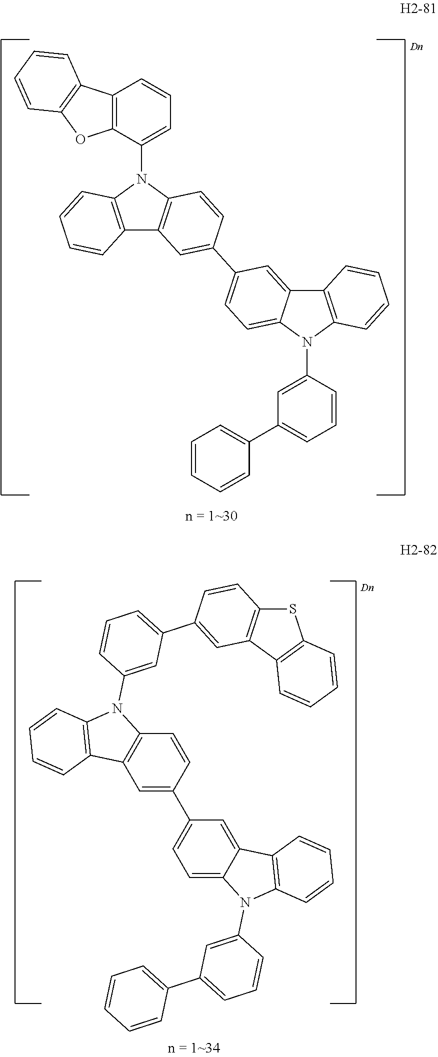

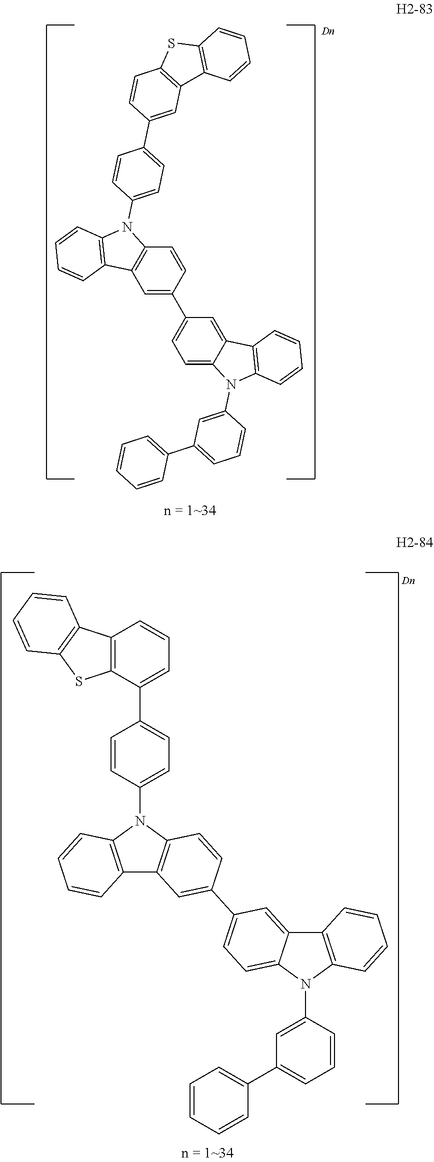

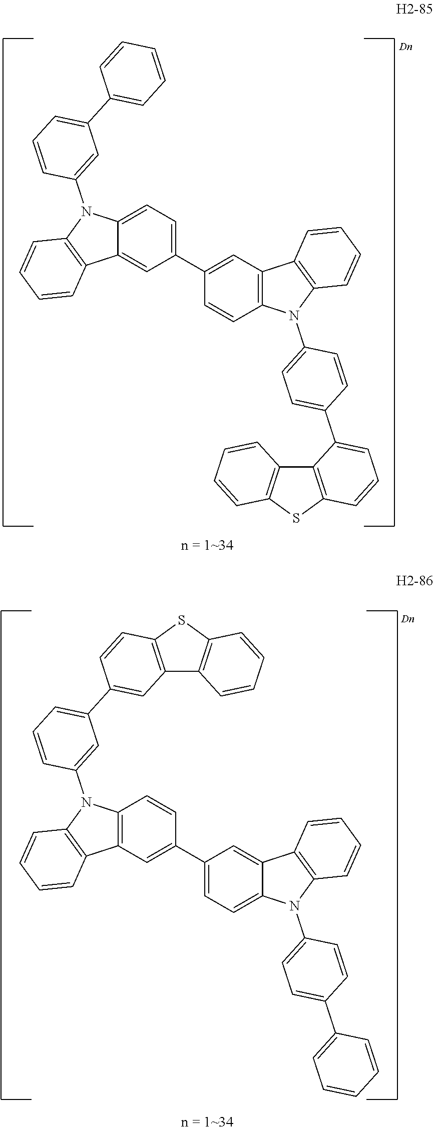

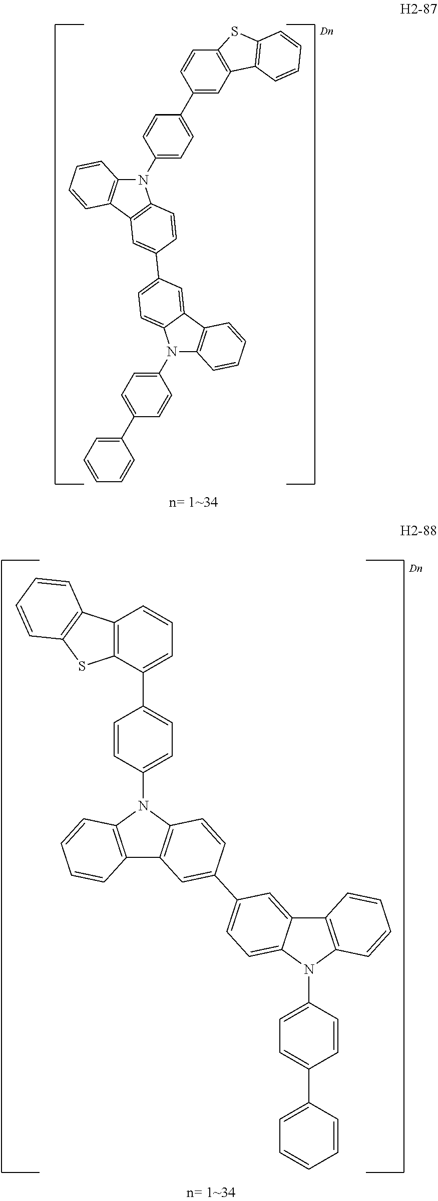

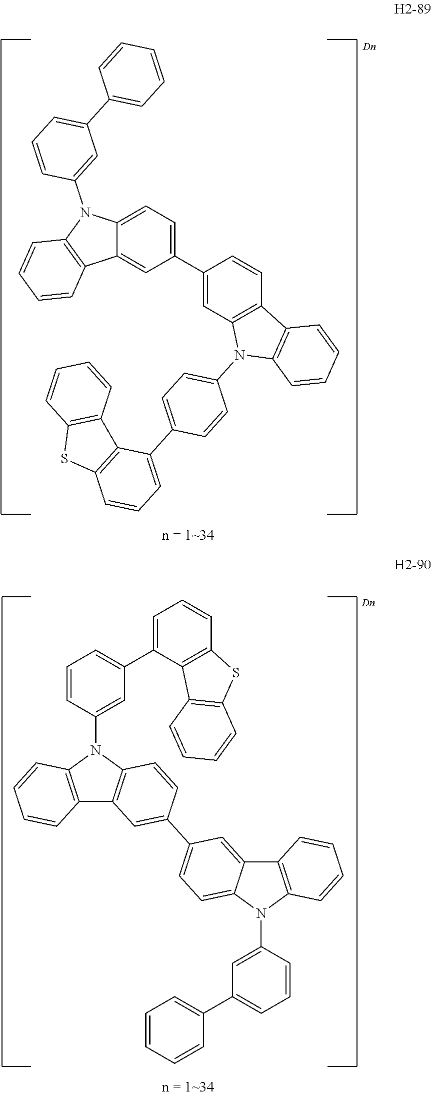

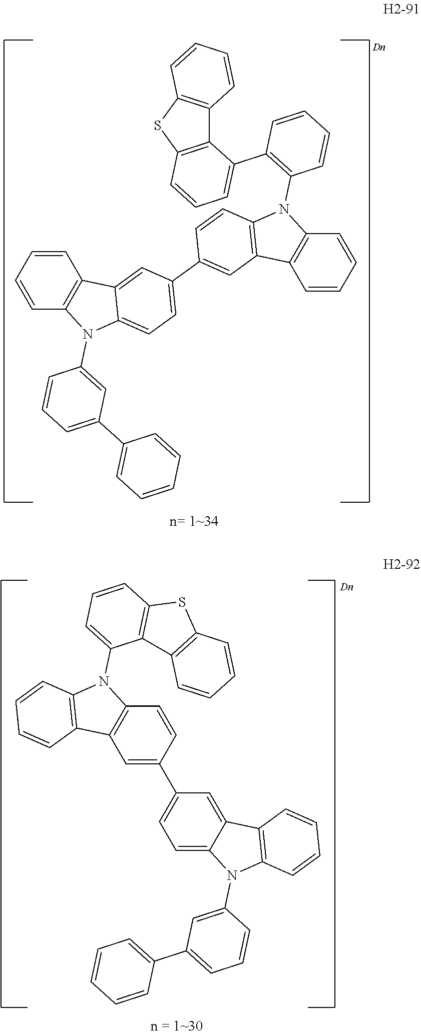

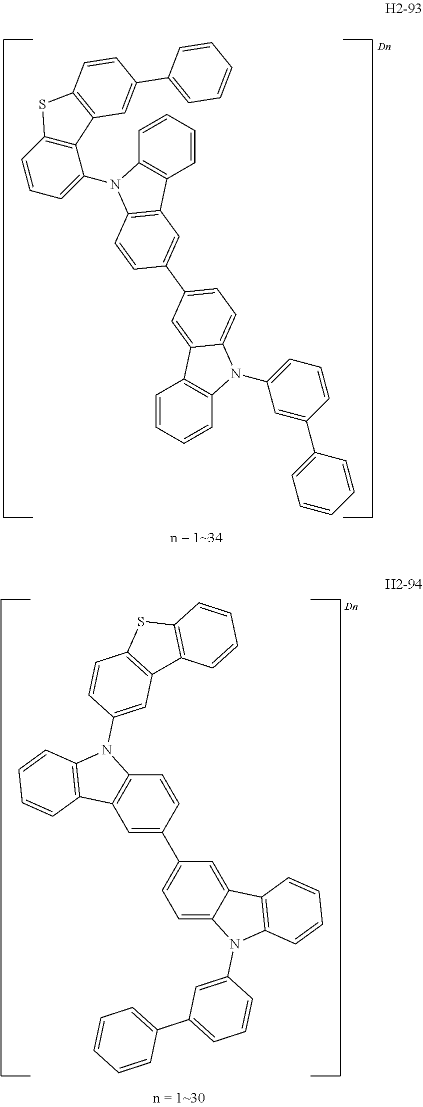

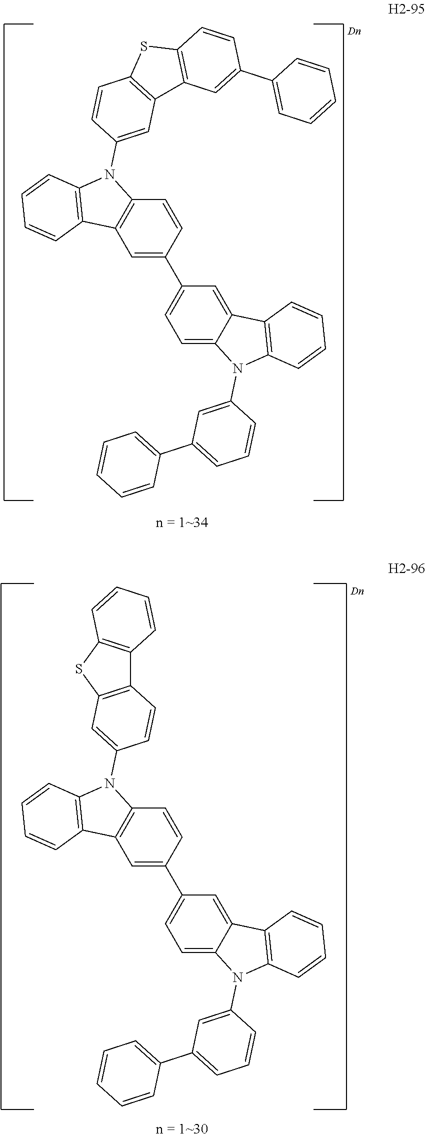

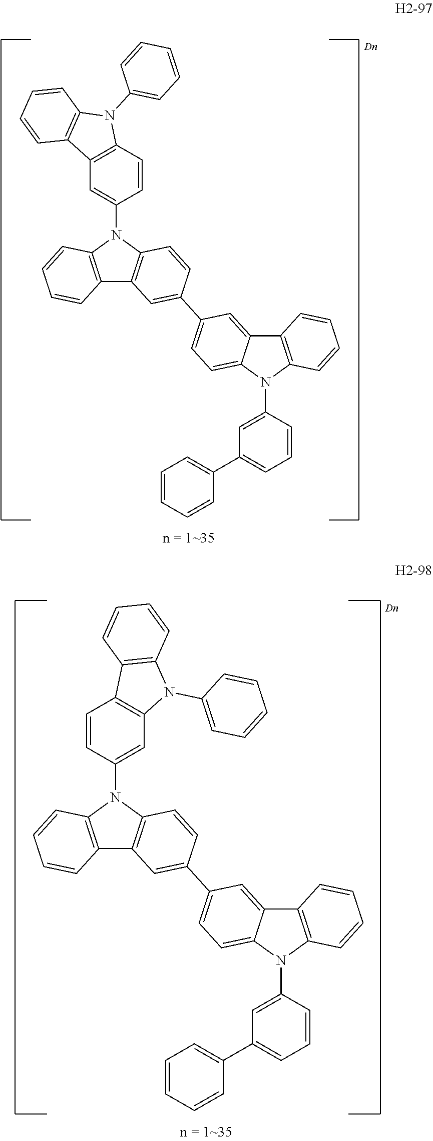

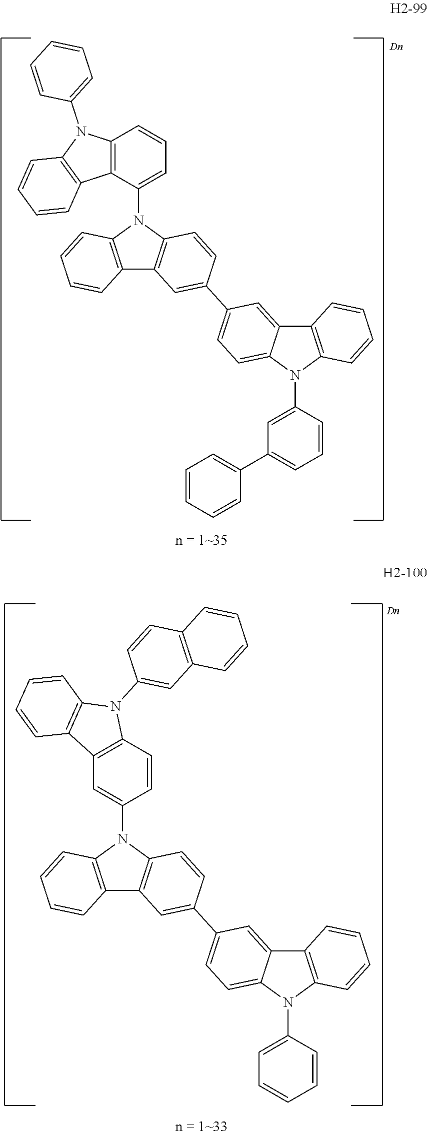

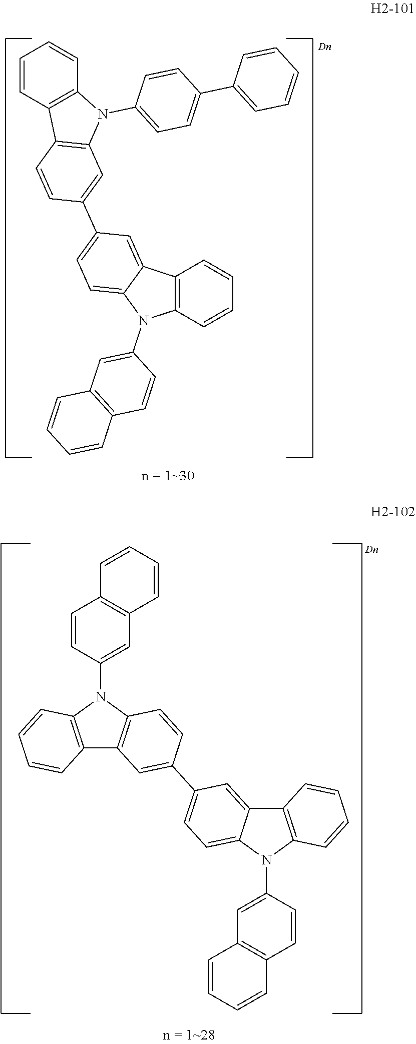

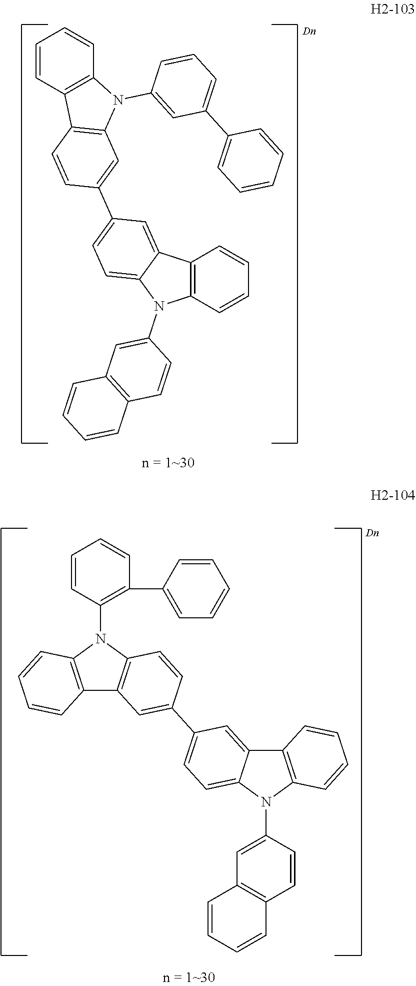

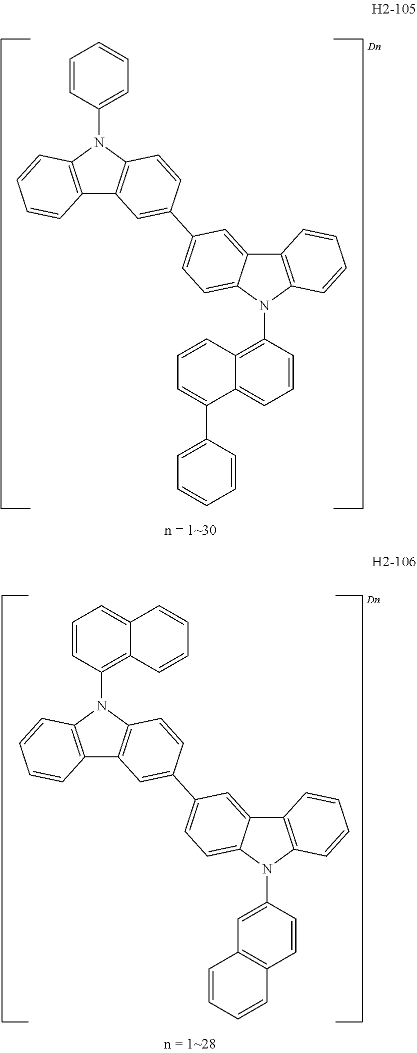

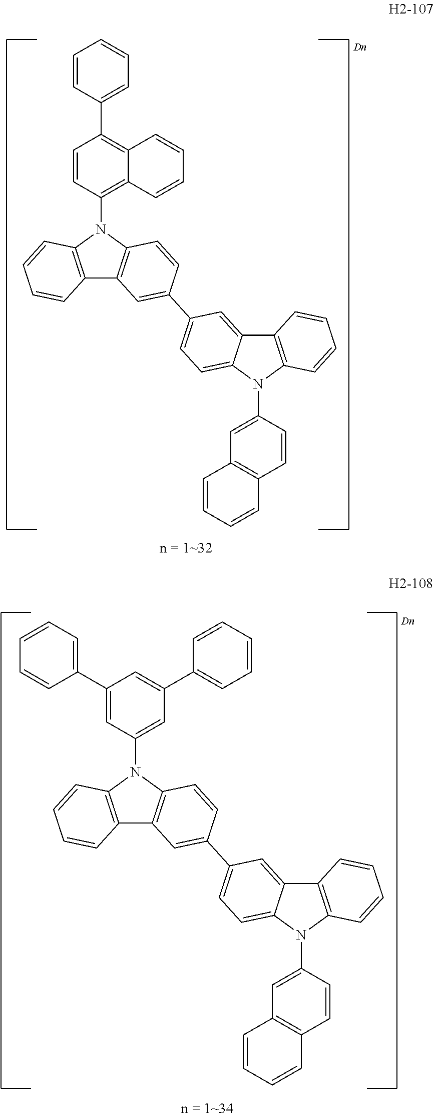

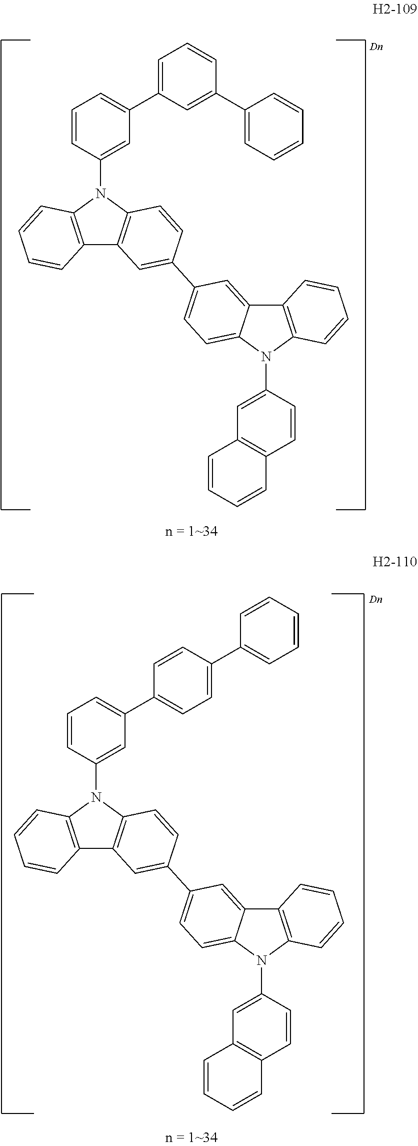

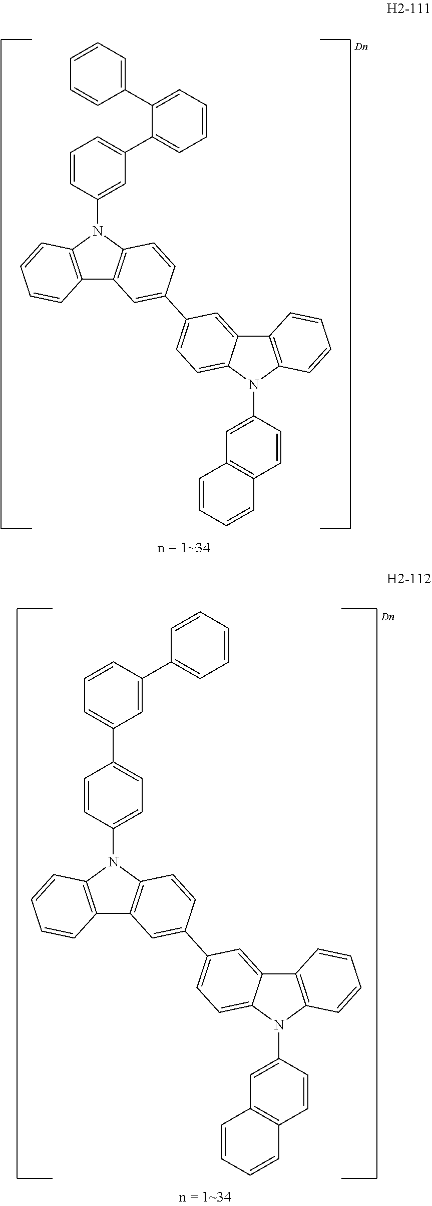

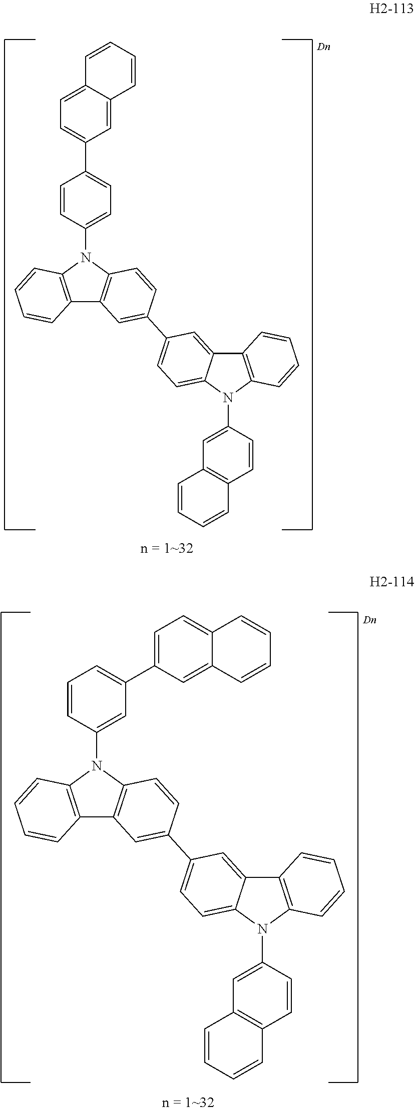

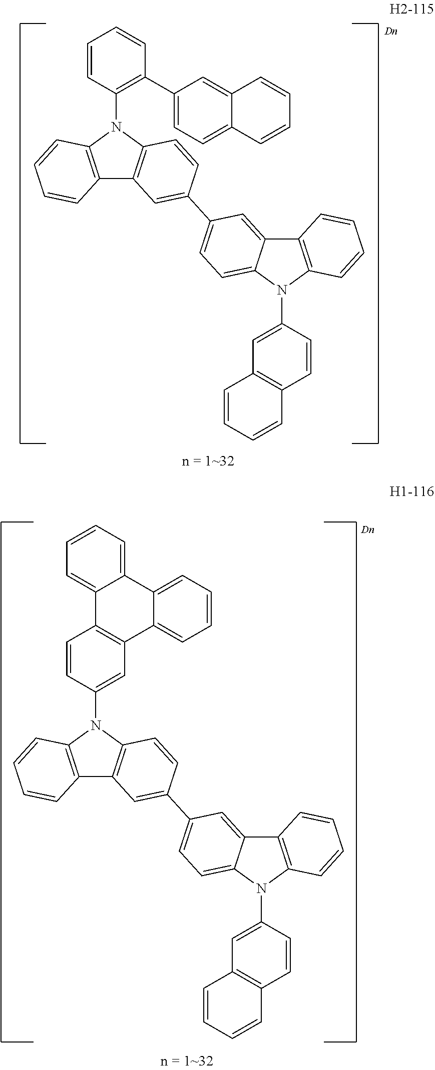

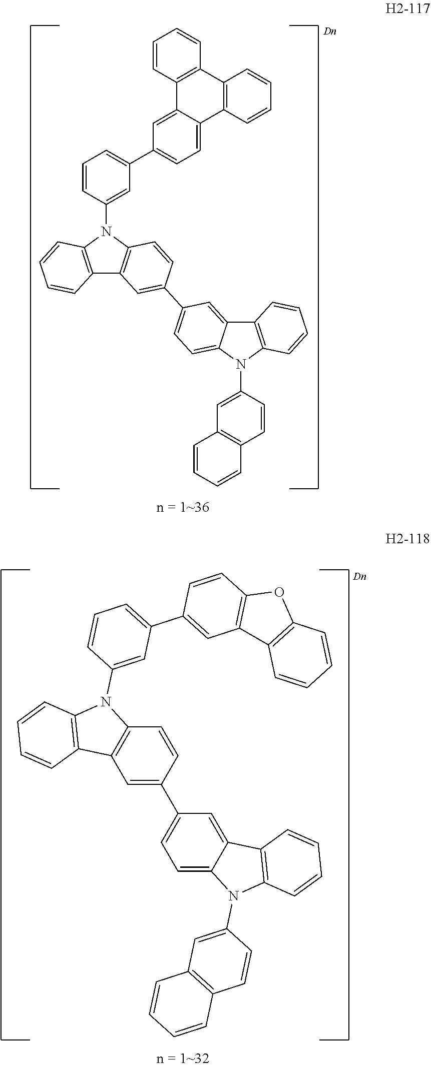

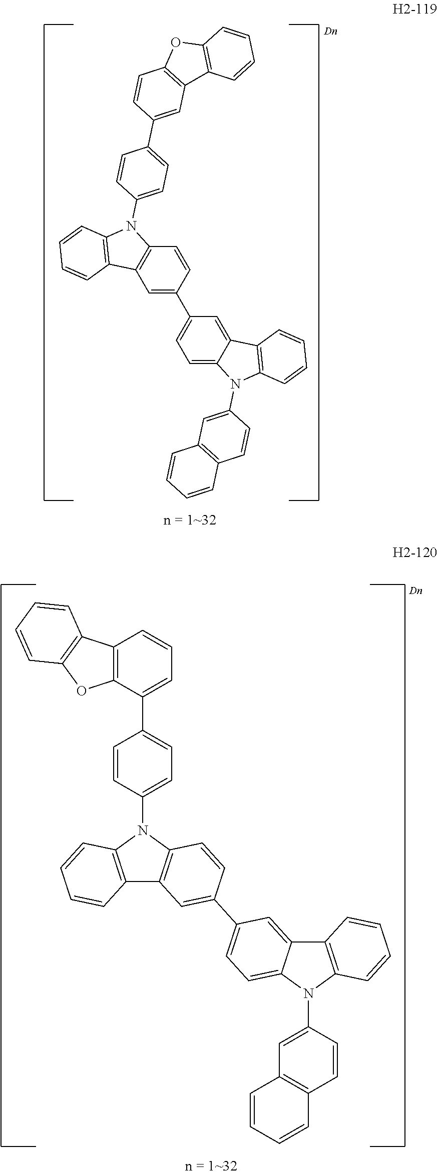

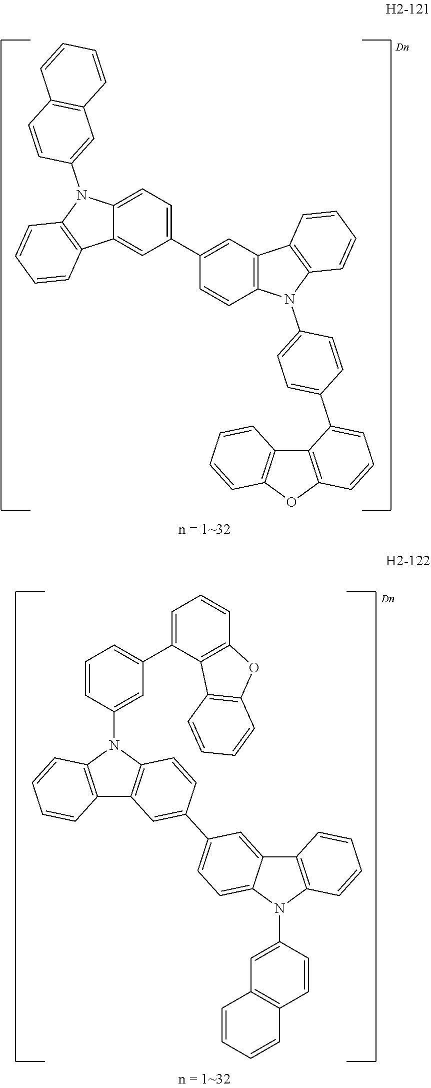

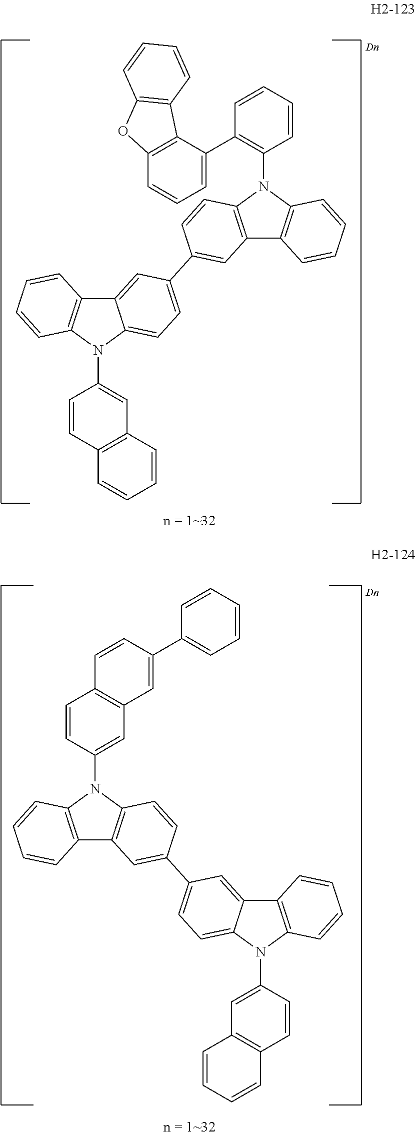

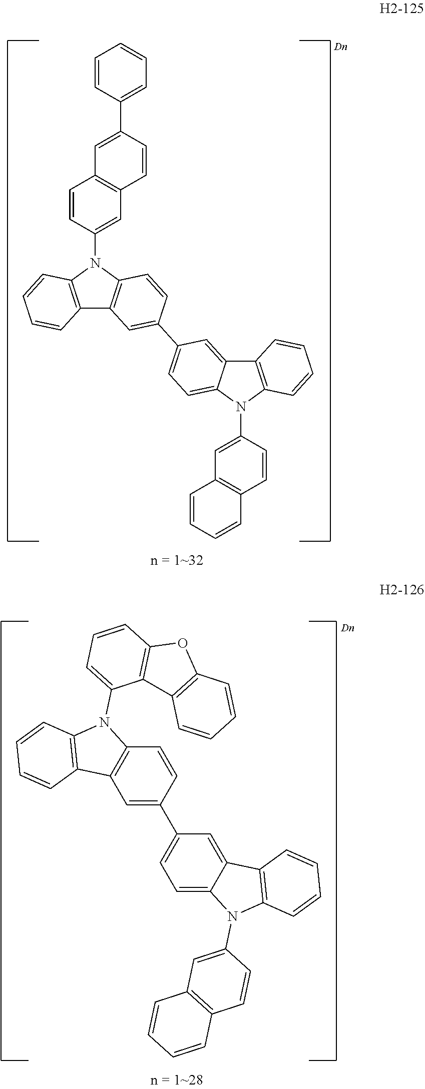

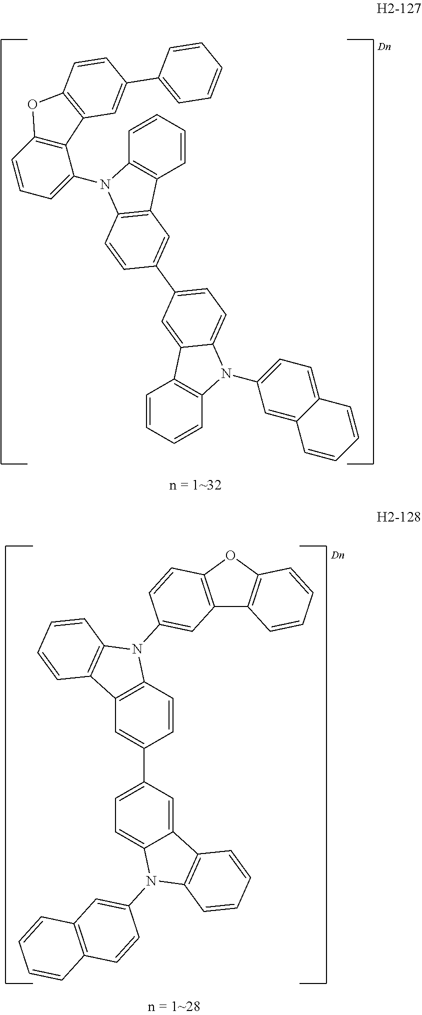

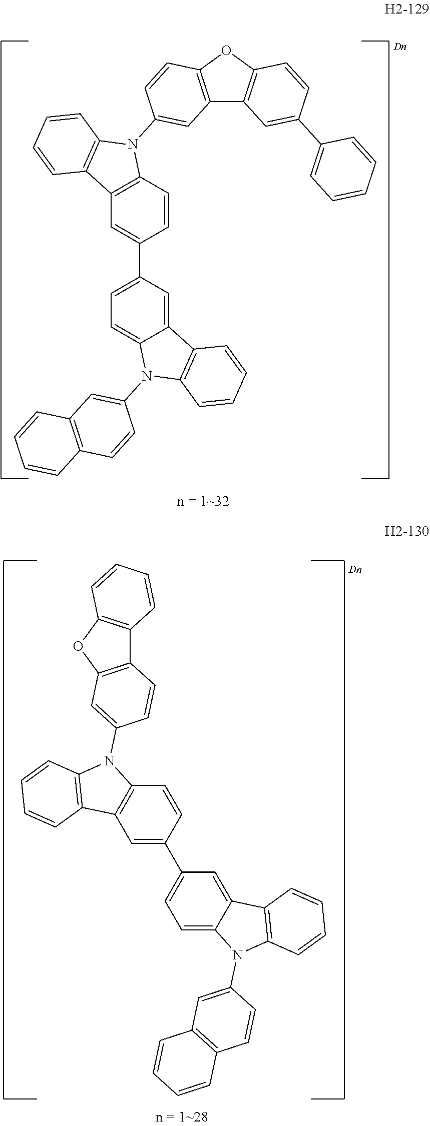

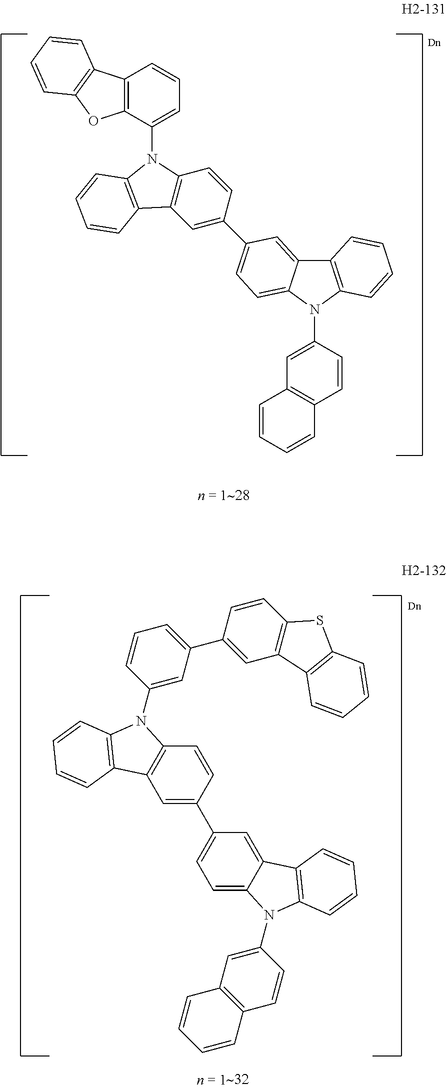

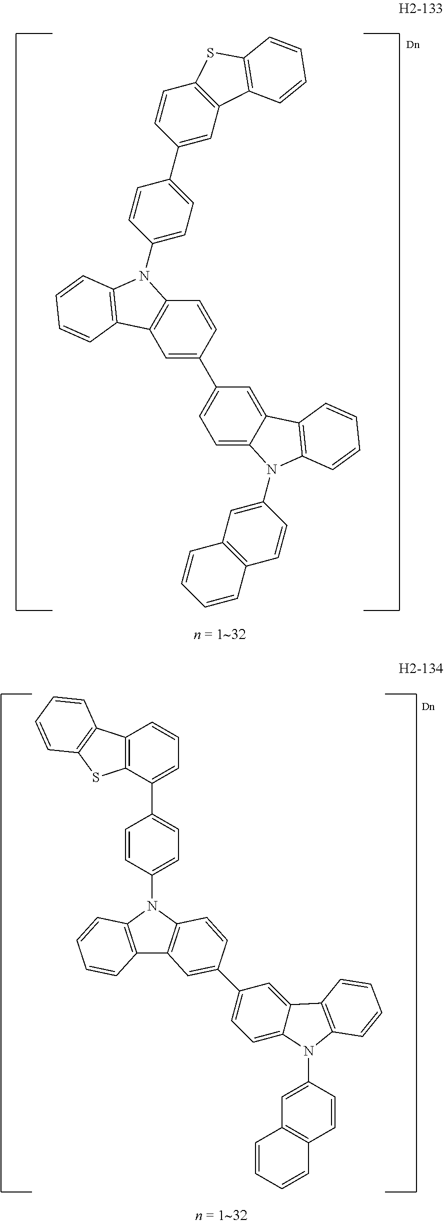

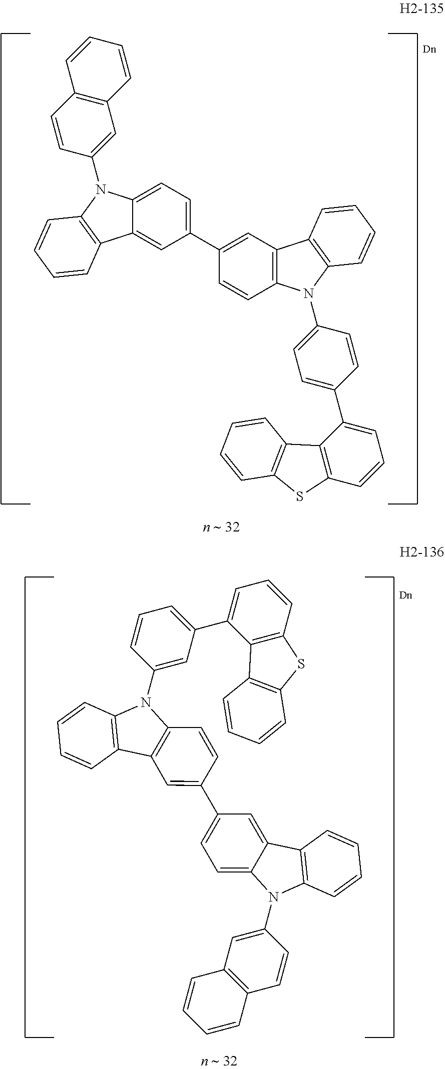

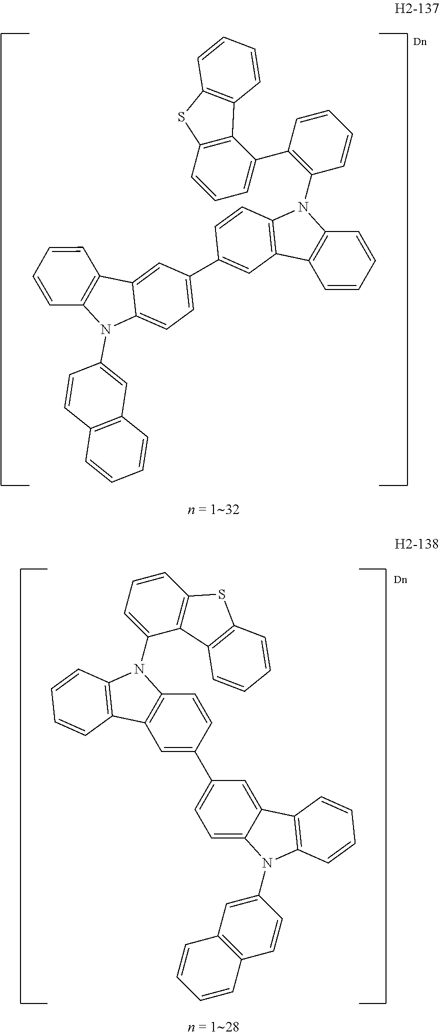

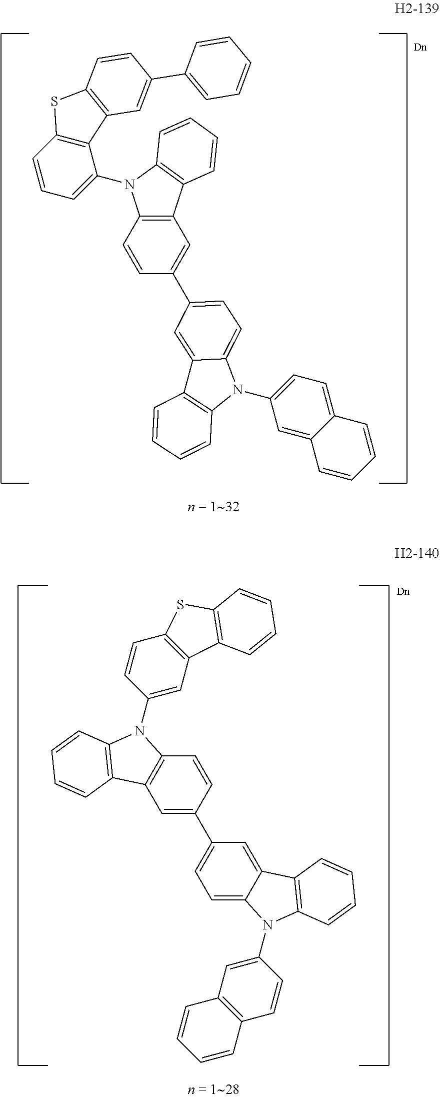

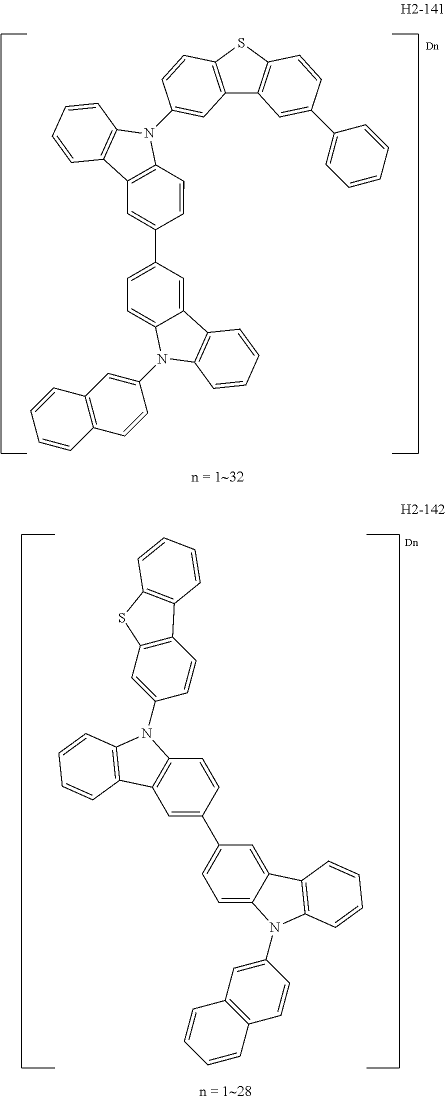

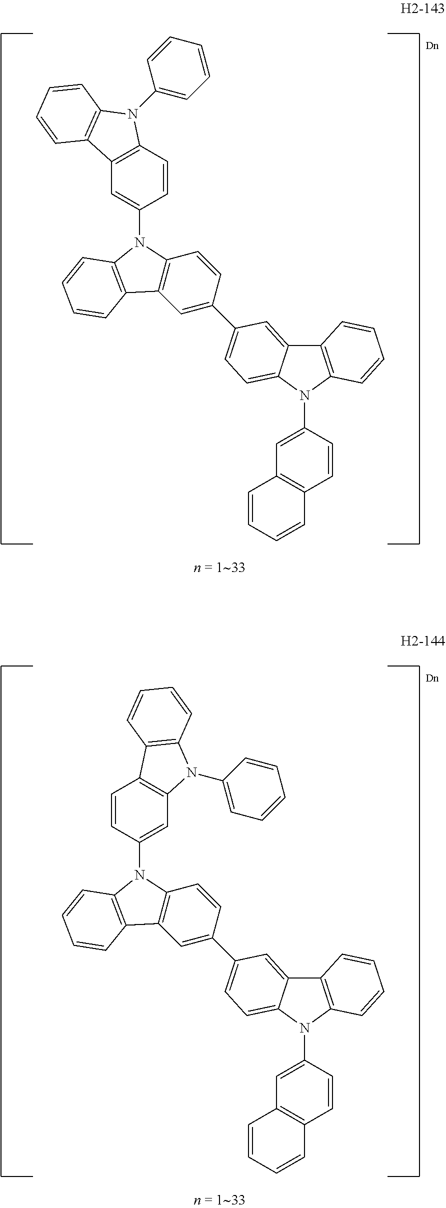

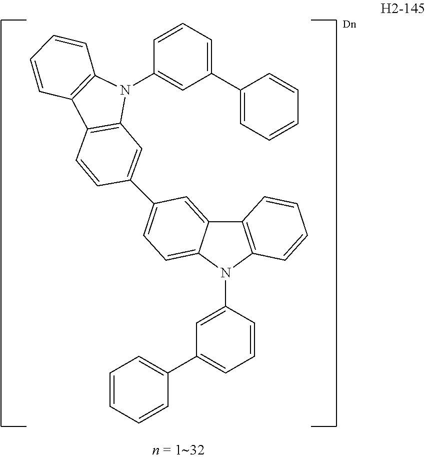

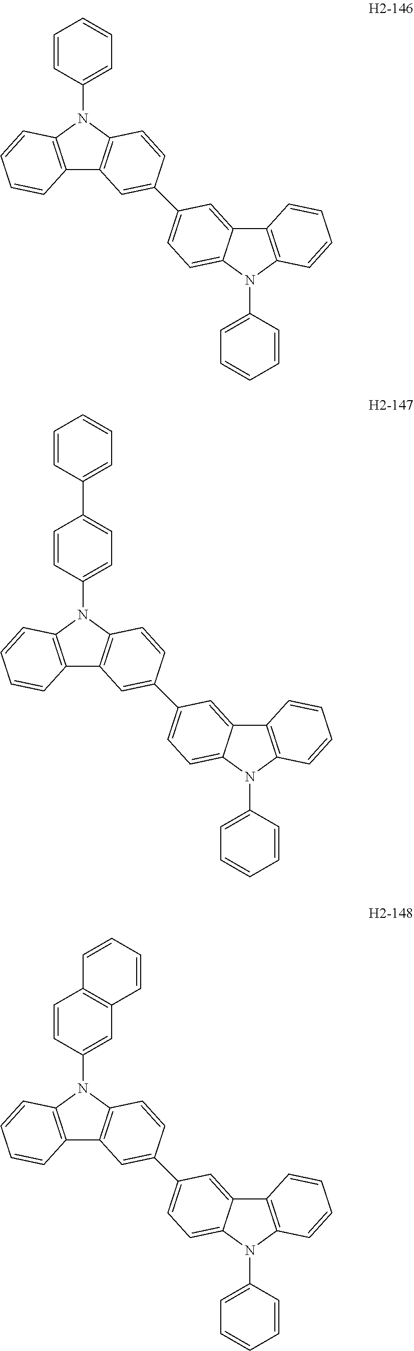 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5ccccc5c4)ccc32)cc1]

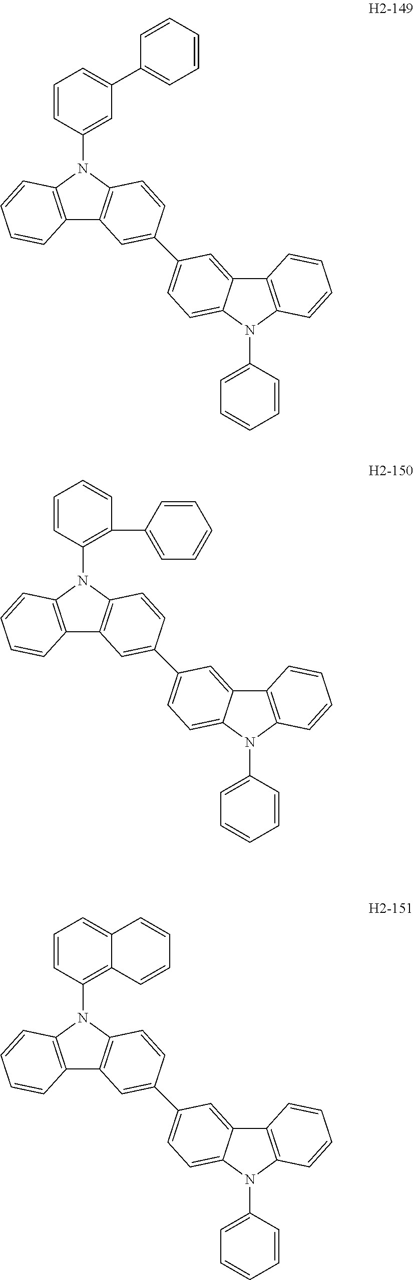 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

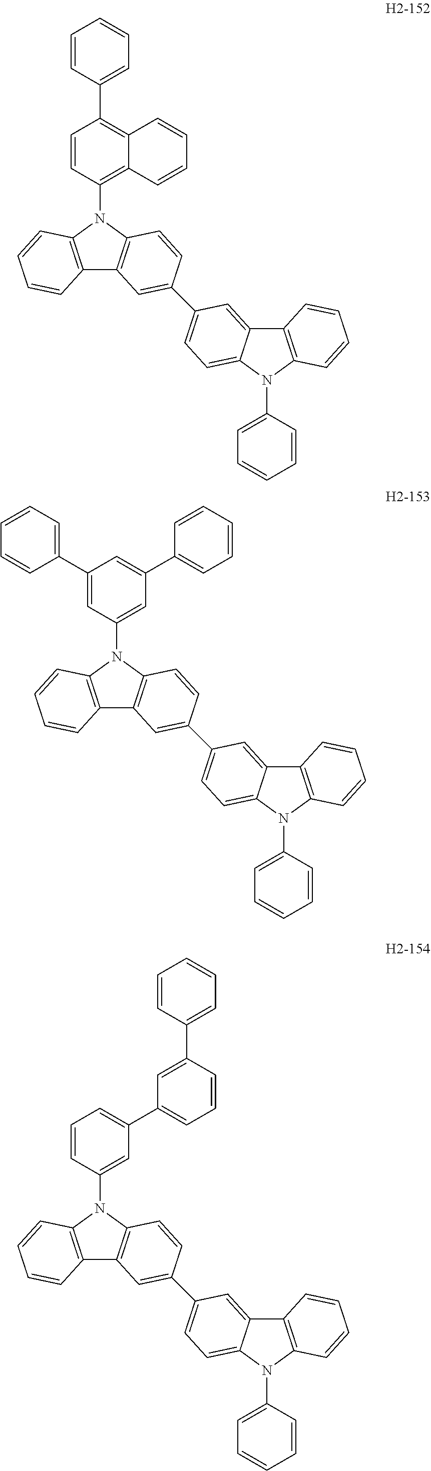 [SMILES: c1ccc(-c2ccc(-n3c4ccccc4c4cc(-c5ccc6c(c5)c5ccccc5n6-c5ccccc5)ccc43)c3ccccc23)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1]

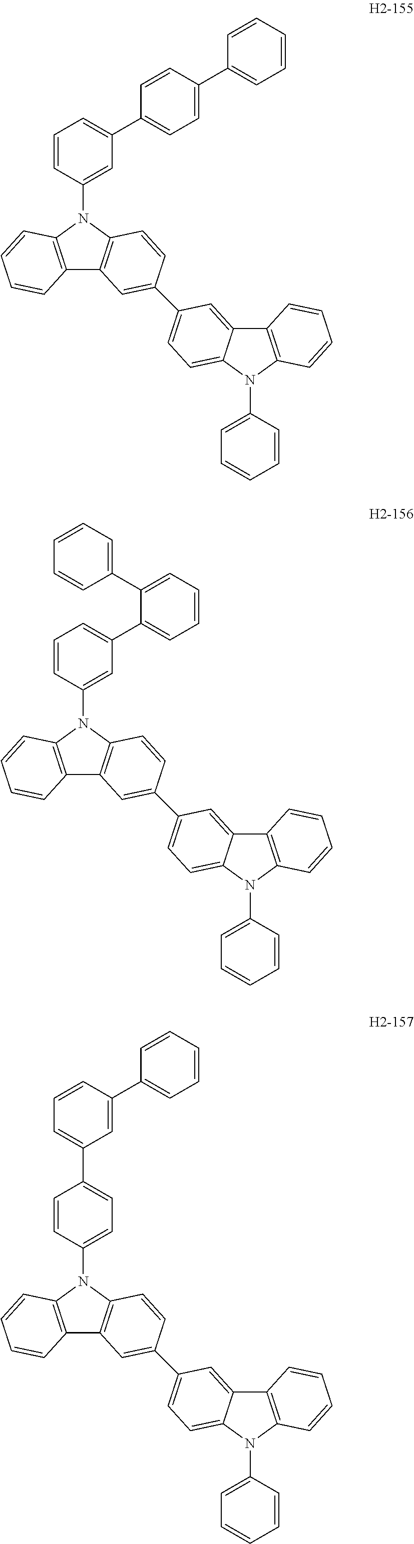 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccccc2-c2cccc(-n3c4ccccc4c4cc(-c5ccc6c(c5)c5ccccc5n6-c5ccccc5)ccc43)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

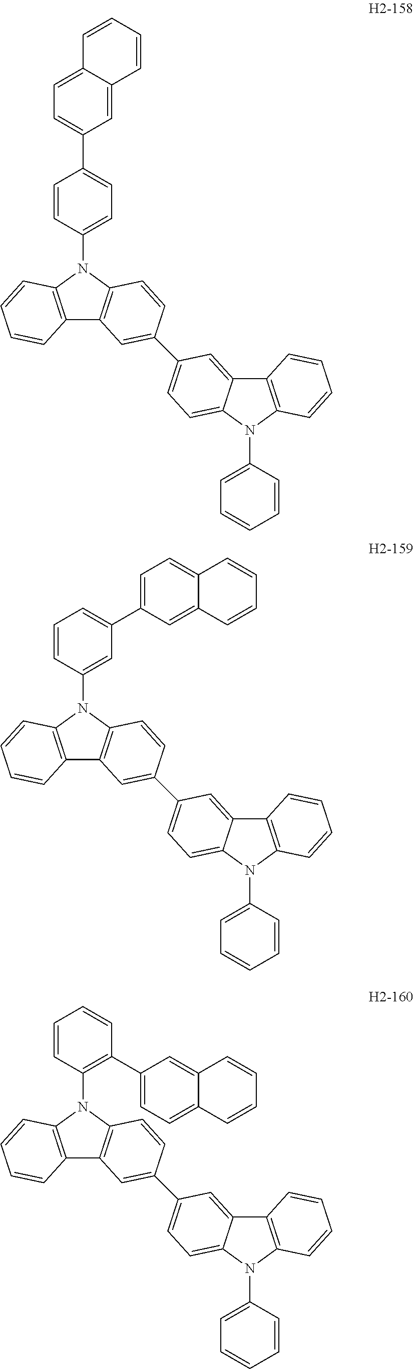

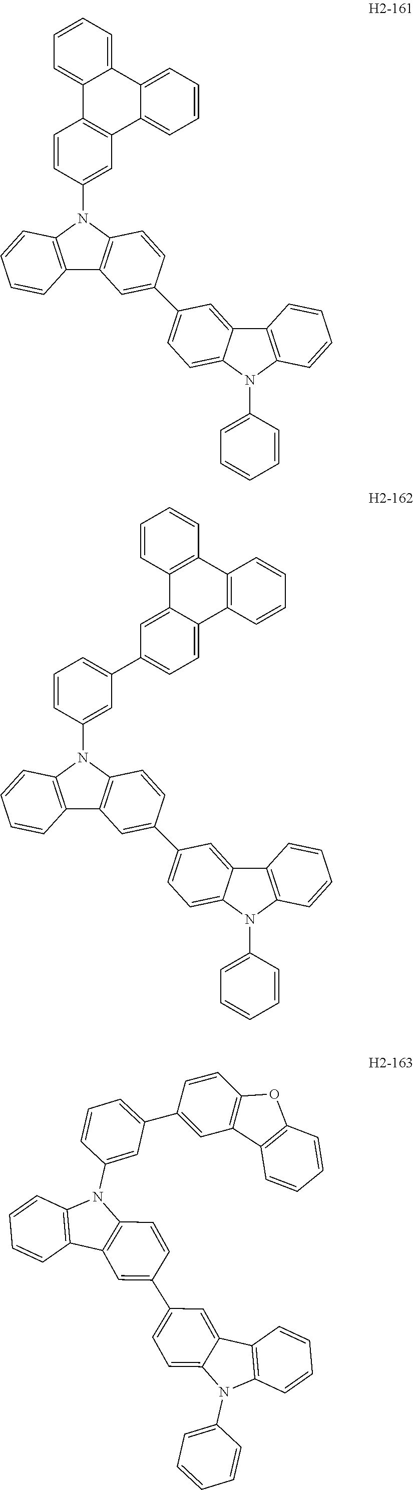 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3cc(-c4ccc5c(c4)c4ccccc4n5-c4cccc(-c5ccc6oc7ccccc7c6c5)c4)ccc32)cc1]

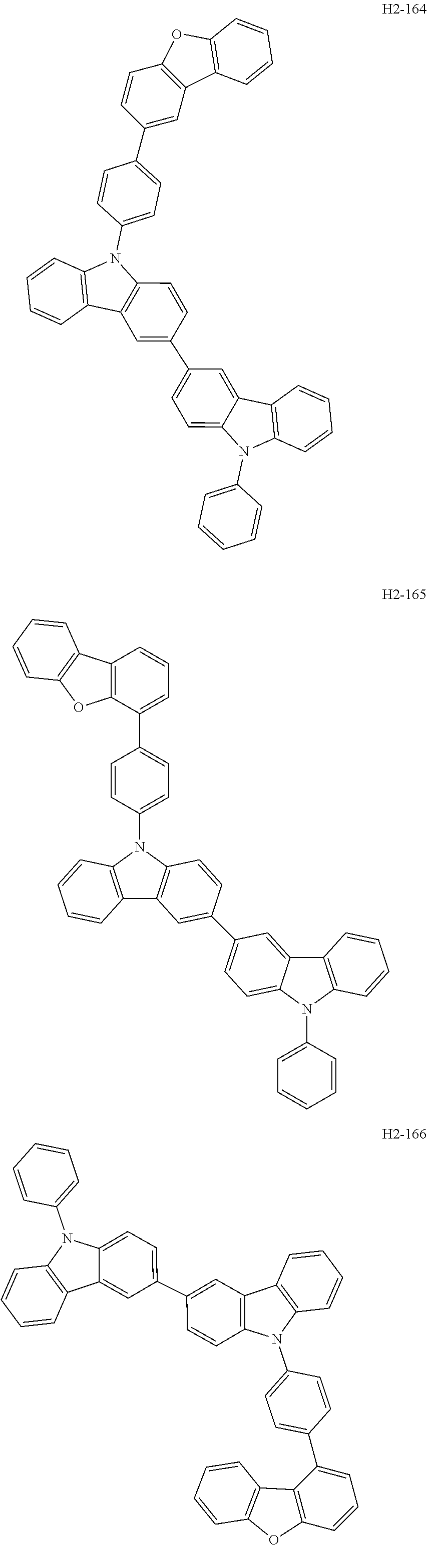 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1]

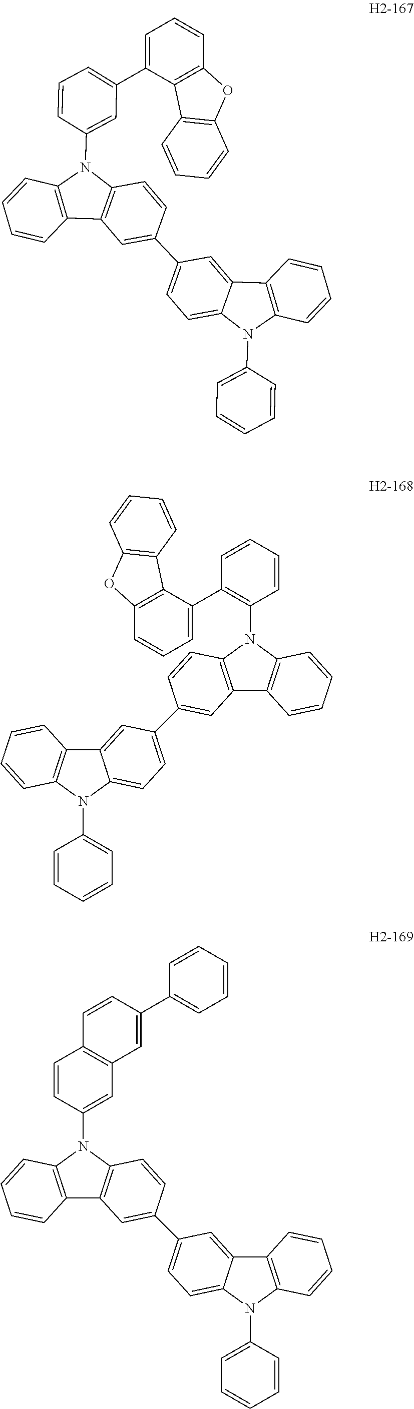 [SMILES: c1ccc(-n2c3ccccc3c3cc(-c4ccc5c(c4)c4ccccc4n5-c4cccc(-c5cccc6oc7ccccc7c56)c4)ccc32)cc1 ; c1ccc(-n2c3ccccc3c3cc(-c4ccc5c(c4)c4ccccc4n5-c4ccccc4-c4cccc5oc6ccccc6c45)ccc32)cc1 ; c1ccc(-c2ccc3ccc(-n4c5ccccc5c5cc(-c6ccc7c(c6)c6ccccc6n7-c6ccccc6)ccc54)cc3c2)cc1]

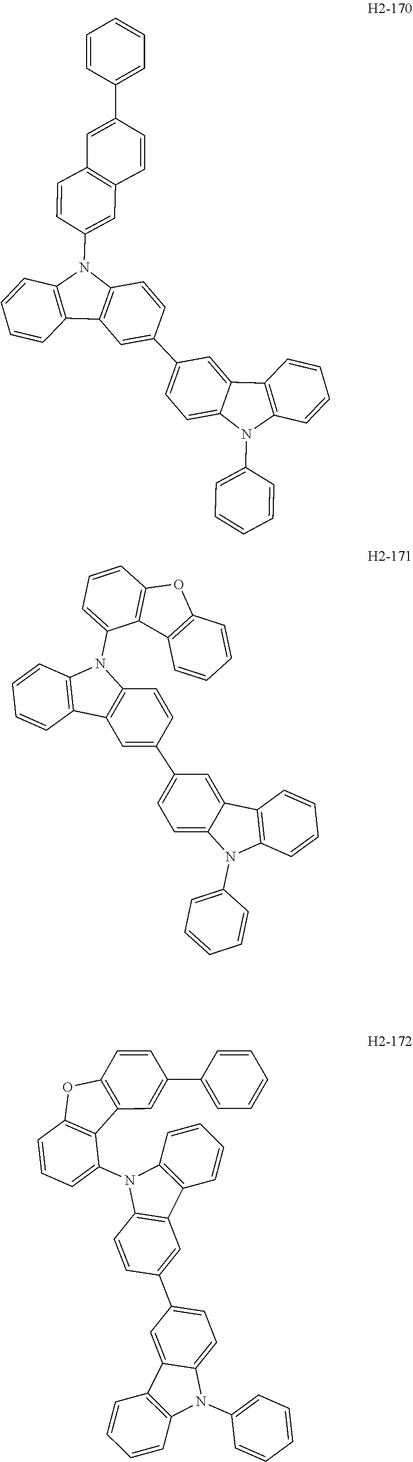 [SMILES: c1ccc(-c2ccc3cc(-n4c5ccccc5c5cc(-c6ccc7c(c6)c6ccccc6n7-c6ccccc6)ccc54)ccc3c2)cc1 ; c1ccc(-n2c3ccccc3c3cc(-c4ccc5c(c4)c4ccccc4n5-c4cccc5oc6ccccc6c45)ccc32)cc1 ; c1ccc(-c2ccc3oc4cccc(-n5c6ccccc6c6cc(-c7ccc8c(c7)c7ccccc7n8-c7ccccc7)ccc65)c4c3c2)cc1]

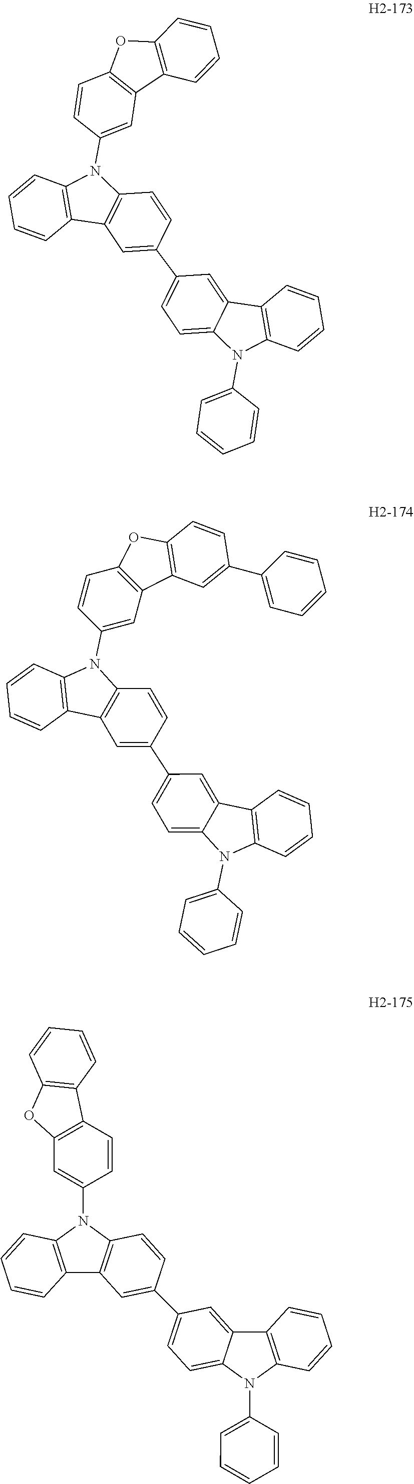 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc3oc4ccc(-n5c6ccccc6c6cc(-c7ccc8c(c7)c7ccccc7n8-c7ccccc7)ccc65)cc4c3c2)cc1 ; c1ccc(-n2c3ccccc3c3cc(-c4ccc5c(c4)c4ccccc4n5-c4ccc5c(c4)oc4ccccc45)ccc32)cc1]

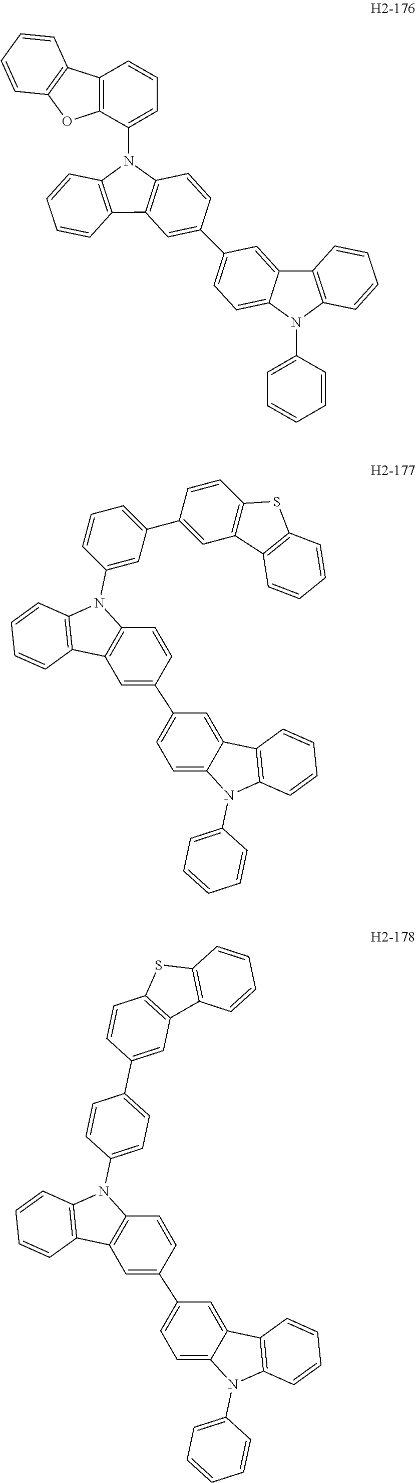 [SMILES: c1ccc(-n2c3ccccc3c3cc(-c4ccc5c(c4)c4ccccc4n5-c4cccc5c4oc4ccccc45)ccc32)cc1 ; c1ccc(-n2c3ccccc3c3cc(-c4ccc5c(c4)c4ccccc4n5-c4cccc(-c5ccc6sc7ccccc7c6c5)c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

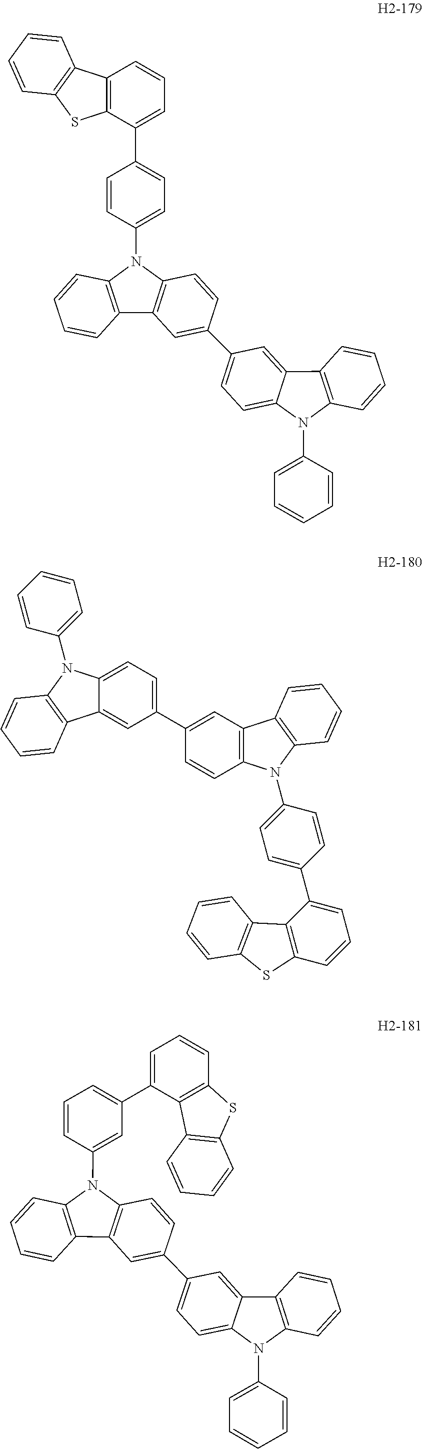 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc(-c5cccc6sc7ccccc7c56)cc4)ccc32)cc1 ; c1ccc(-n2c3ccccc3c3cc(-c4ccc5c(c4)c4ccccc4n5-c4cccc(-c5cccc6sc7ccccc7c56)c4)ccc32)cc1]

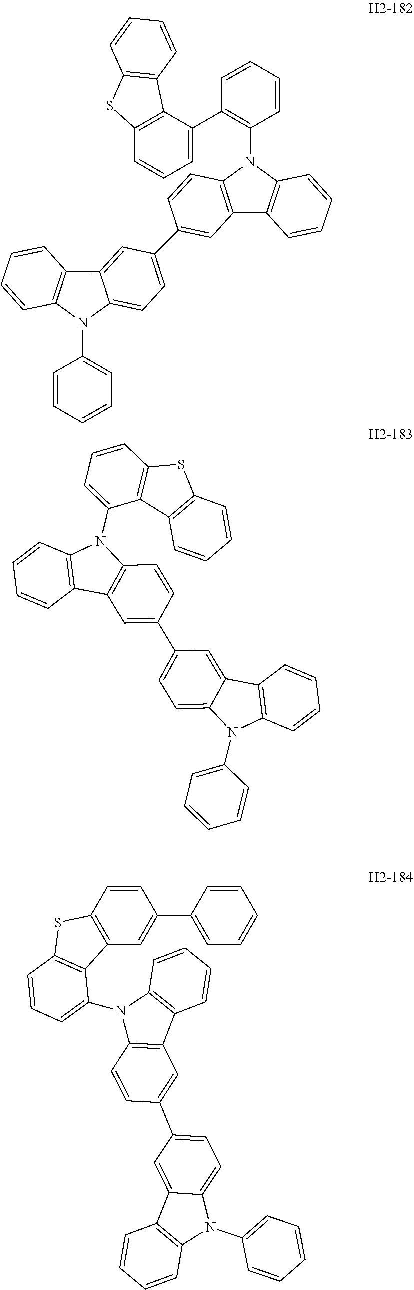

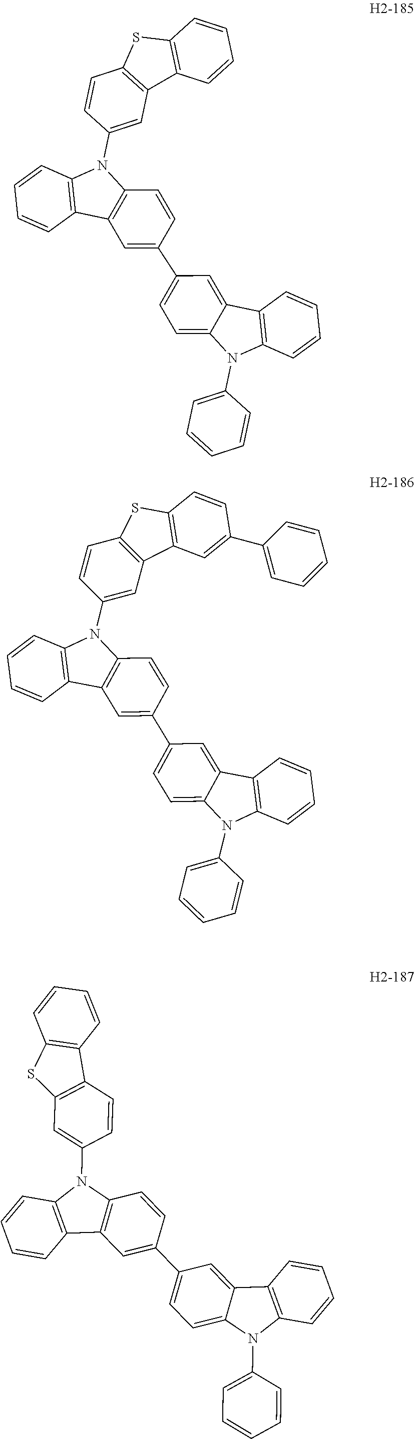

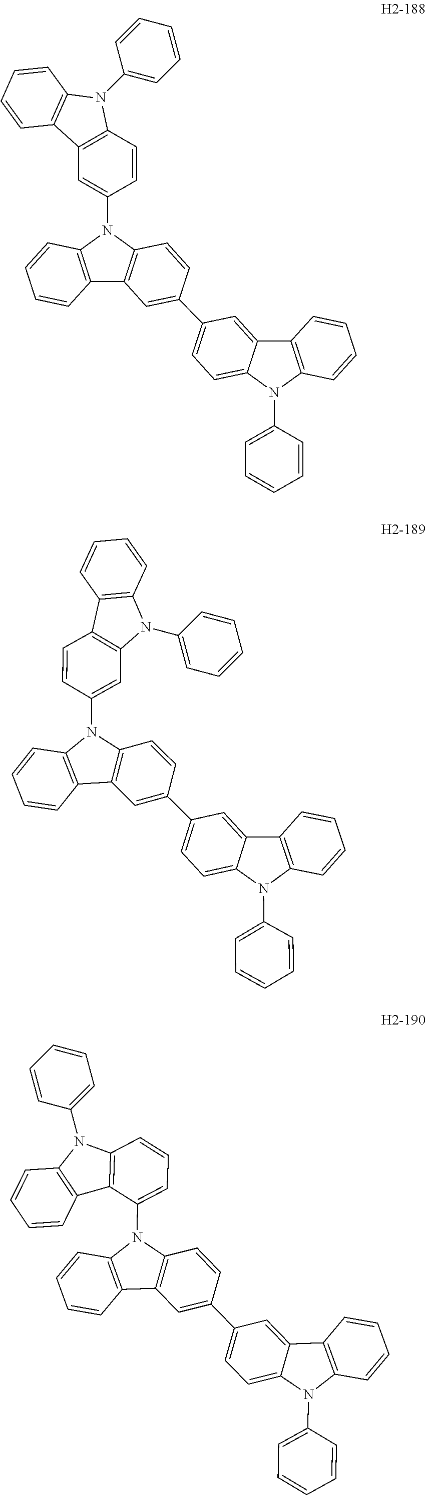 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4cccc5c4c4ccccc4n5-c4ccccc4)ccc32)cc1]

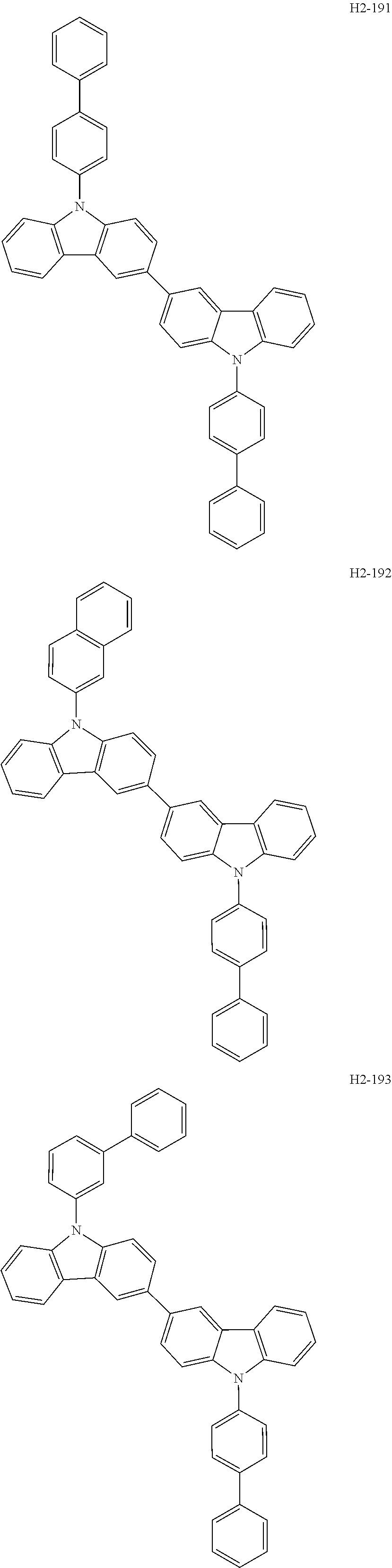 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

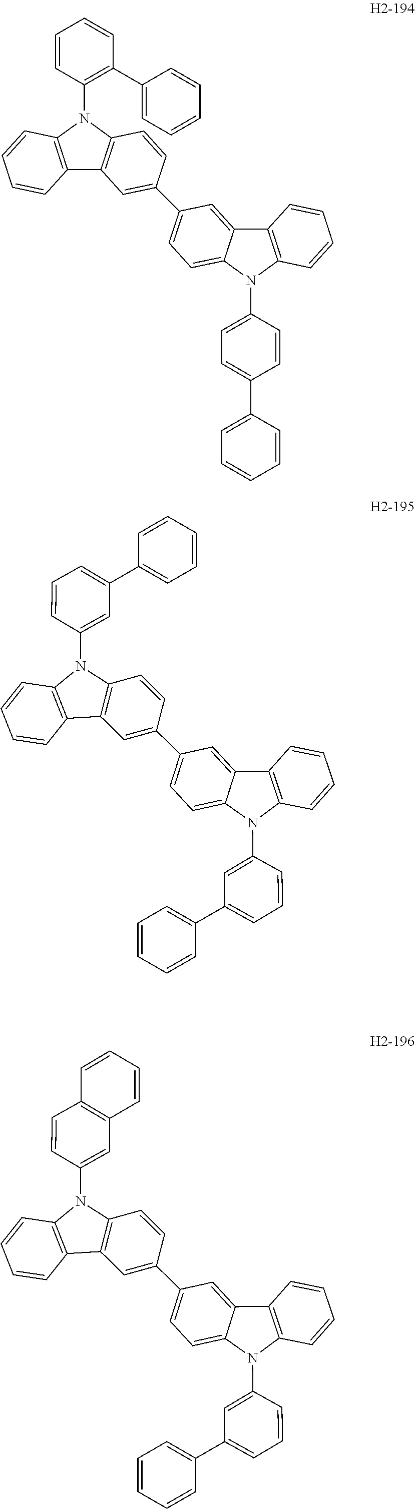 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1]

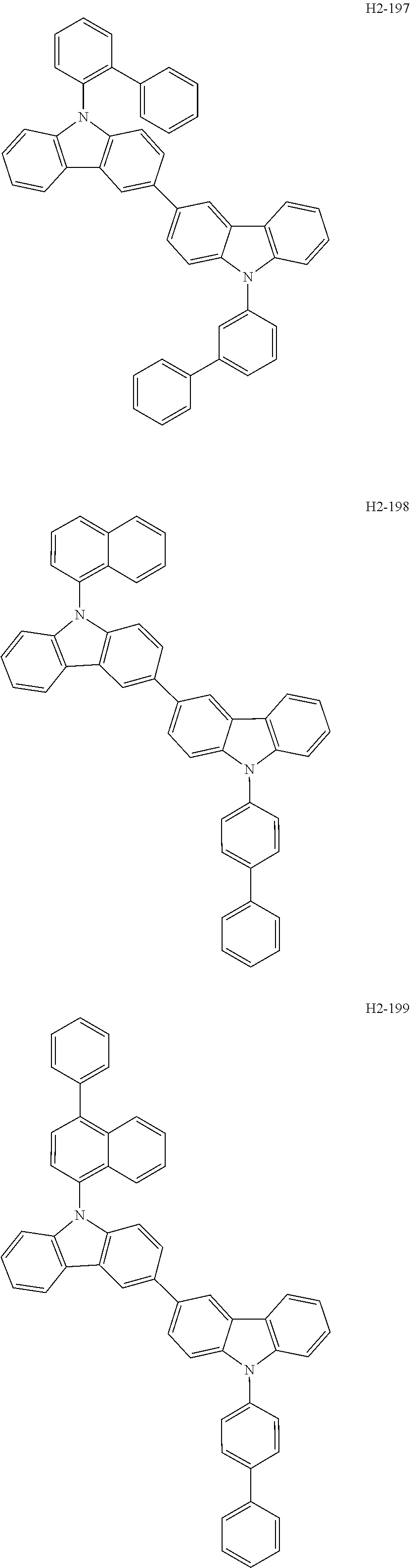 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccccc4c4cc(-c5ccc6c(c5)c5ccccc5n6-c5ccc(-c6ccccc6)c6ccccc56)ccc43)cc2)cc1]

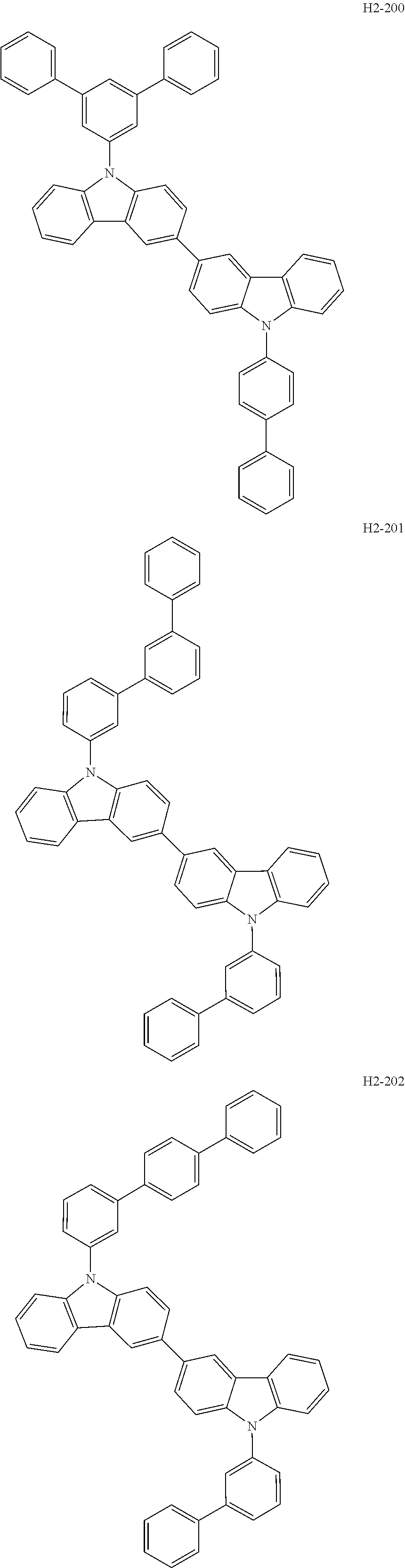 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1]

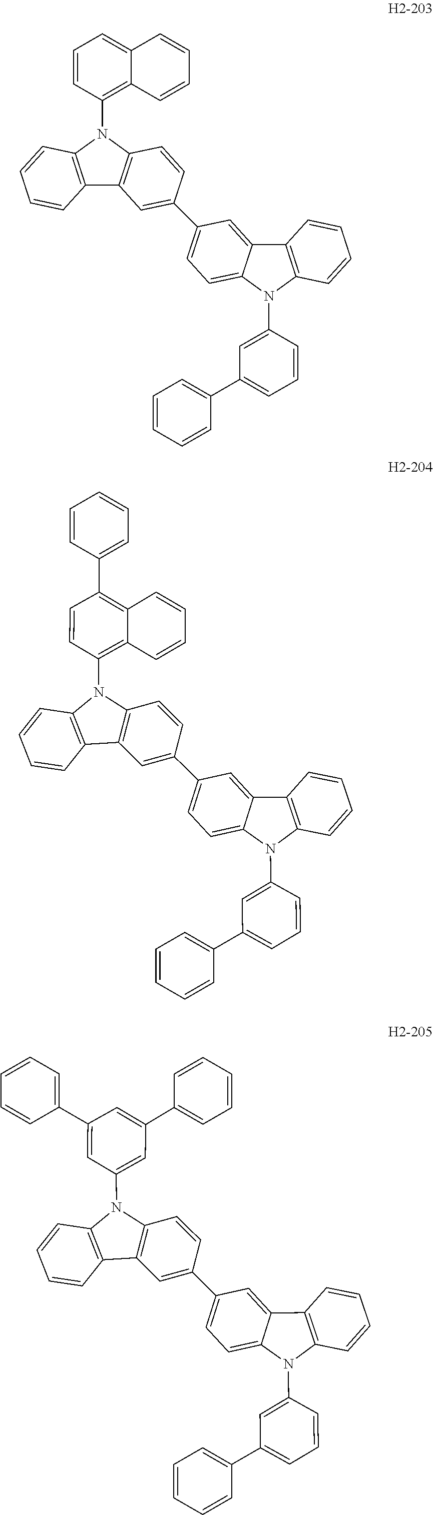 [SMILES: c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc(-c6ccccc6)c6ccccc56)ccc43)c2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

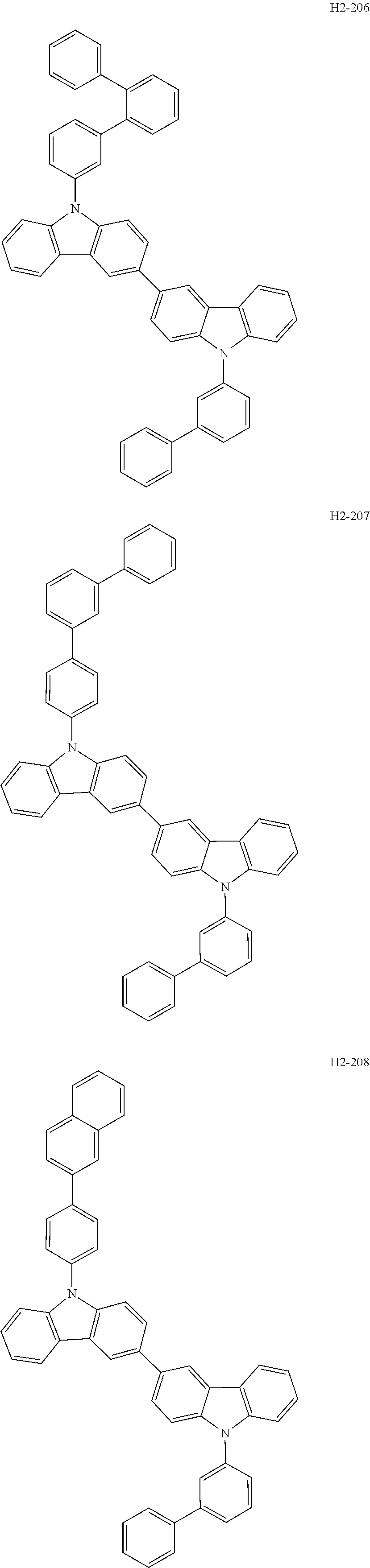

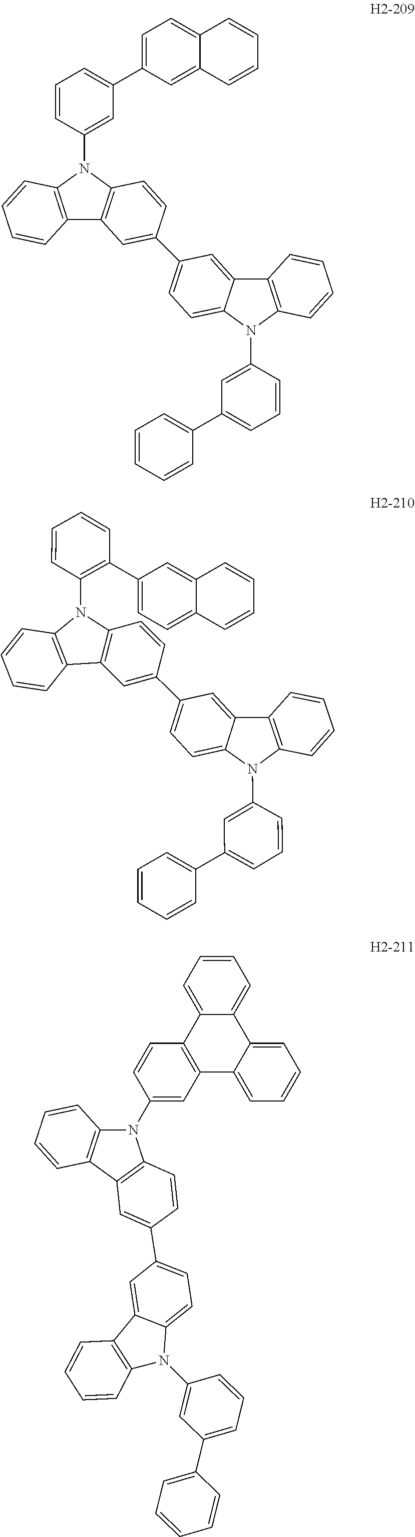 [SMILES: c1ccc(-c2cccc(-n3c4ccccc4c4cc(-c5ccc6c(c5)c5ccccc5n6-c5cccc(-c6ccc7ccccc7c6)c5)ccc43)c2)cc1 ; c1ccc(-c2cccc(-n3c4ccccc4c4cc(-c5ccc6c(c5)c5ccccc5n6-c5ccccc5-c5ccc6ccccc6c5)ccc43)c2)cc1 ; c1ccc(-c2cccc(-n3c4ccccc4c4cc(-c5ccc6c(c5)c5ccccc5n6-c5ccc6c7ccccc7c7ccccc7c6c5)ccc43)c2)cc1]

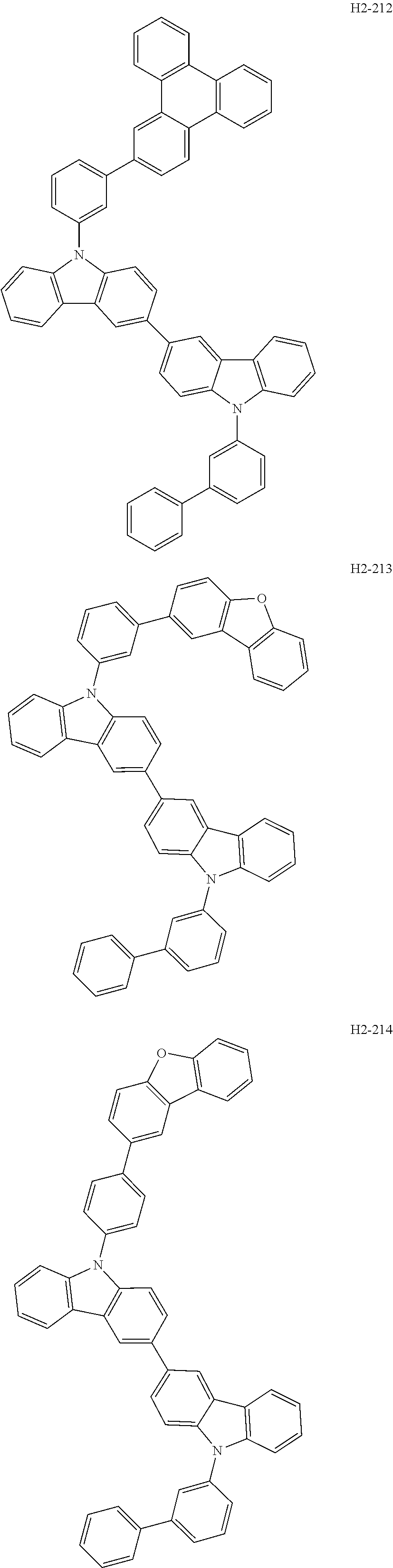

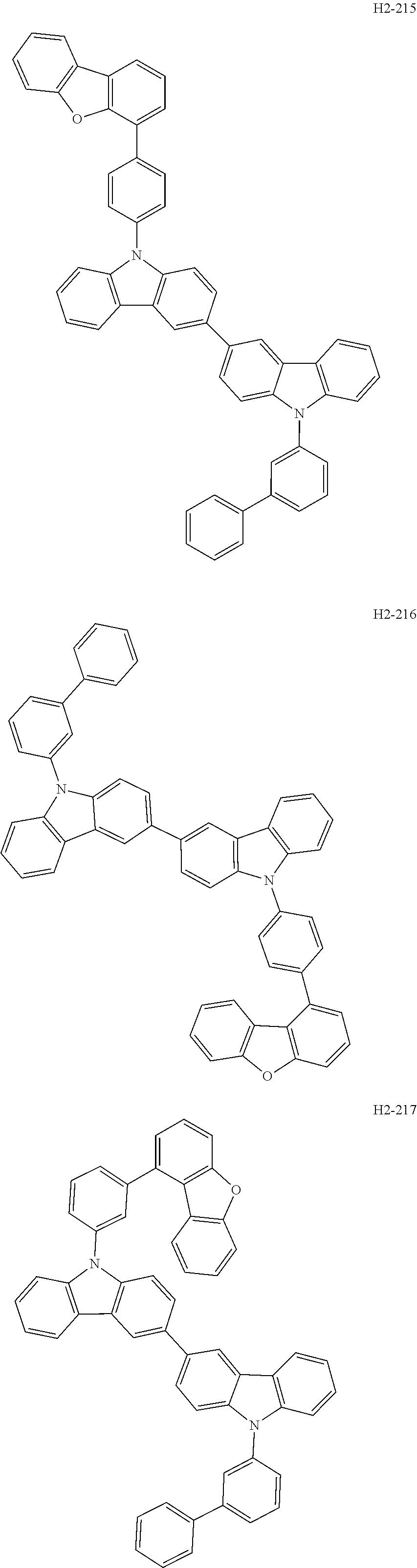 [SMILES: c1ccc(-c2cccc(-n3c4ccccc4c4cc(-c5ccc6c(c5)c5ccccc5n6-c5ccc(-c6cccc7c6oc6ccccc67)cc5)ccc43)c2)cc1 ; c1ccc(-c2cccc(-n3c4ccccc4c4cc(-c5ccc6c(c5)c5ccccc5n6-c5ccc(-c6cccc7oc8ccccc8c67)cc5)ccc43)c2)cc1 ; c1ccc(-c2cccc(-n3c4ccccc4c4cc(-c5ccc6c(c5)c5ccccc5n6-c5cccc(-c6cccc7oc8ccccc8c67)c5)ccc43)c2)cc1]

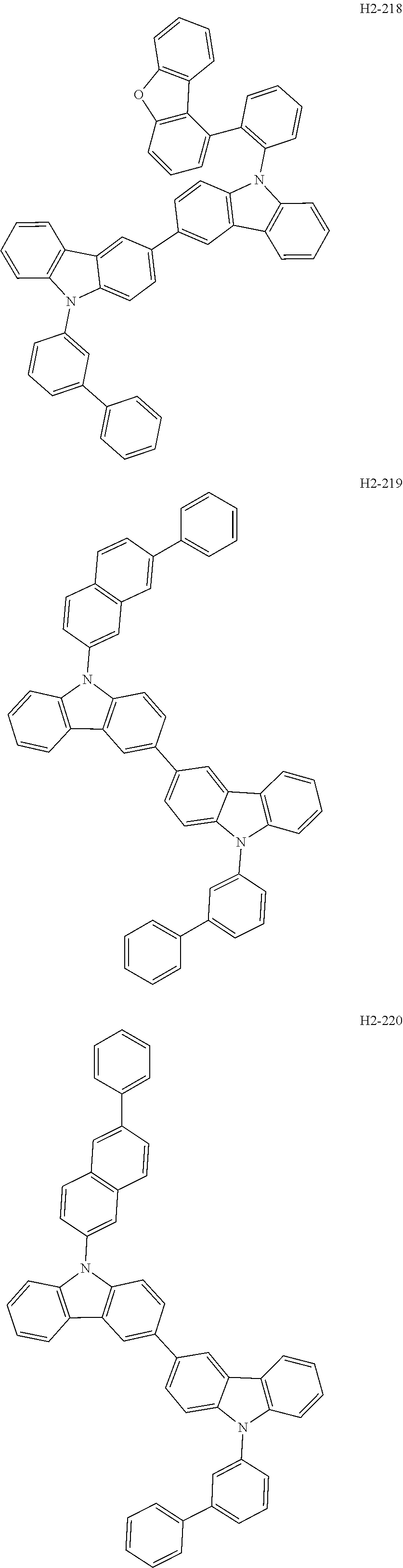 [SMILES: c1ccc(-c2cccc(-n3c4ccccc4c4cc(-c5ccc6c(c5)c5ccccc5n6-c5ccccc5-c5cccc6oc7ccccc7c56)ccc43)c2)cc1 ; c1ccc(-c2cccc(-n3c4ccccc4c4cc(-c5ccc6c(c5)c5ccccc5n6-c5ccc6ccc(-c7ccccc7)cc6c5)ccc43)c2)cc1 ; c1ccc(-c2cccc(-n3c4ccccc4c4cc(-c5ccc6c(c5)c5ccccc5n6-c5ccc6cc(-c7ccccc7)ccc6c5)ccc43)c2)cc1]

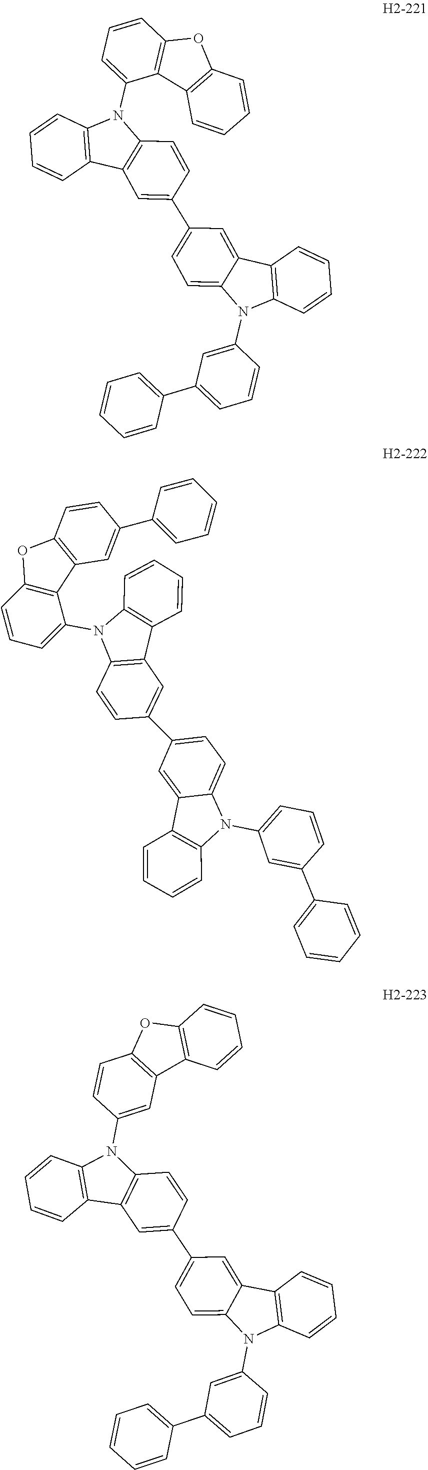 [SMILES: c1ccc(-c2cccc(-n3c4ccccc4c4cc(-c5ccc6c(c5)c5ccccc5n6-c5cccc6oc7ccccc7c56)ccc43)c2)cc1 ; c1ccc(-c2cccc(-n3c4ccccc4c4cc(-c5ccc6c(c5)c5ccccc5n6-c5cccc6oc7ccc(-c8ccccc8)cc7c56)ccc43)c2)cc1 ; c1ccc(-c2cccc(-n3c4ccccc4c4cc(-c5ccc6c(c5)c5ccccc5n6-c5ccc6oc7ccccc7c6c5)ccc43)c2)cc1]

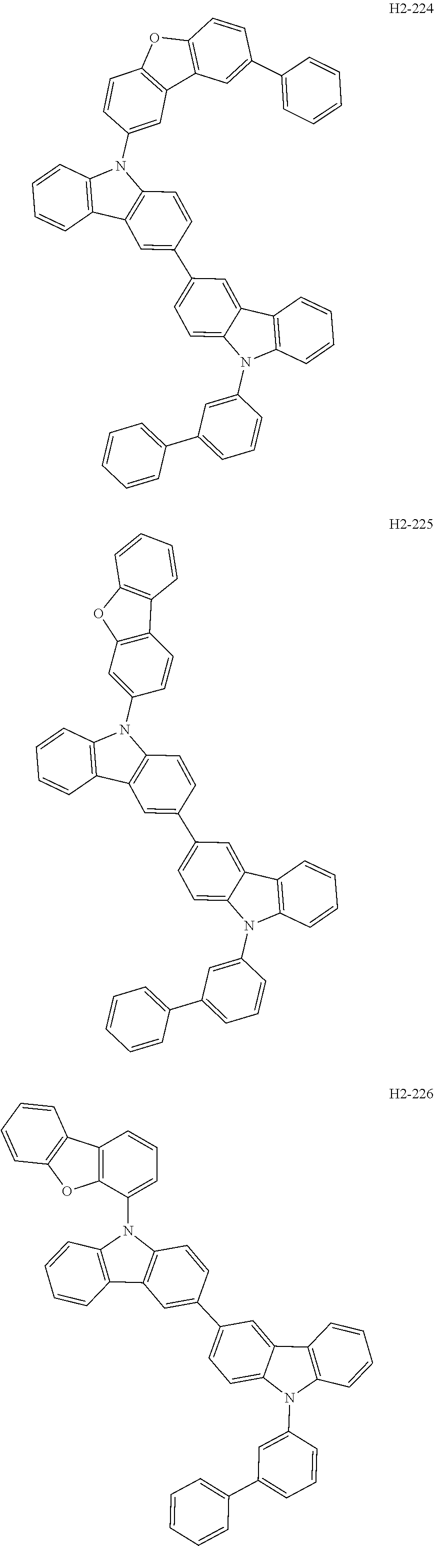 [SMILES: c1ccc(-c2cccc(-n3c4ccccc4c4cc(-c5ccc6c(c5)c5ccccc5n6-c5ccc6oc7ccc(-c8ccccc8)cc7c6c5)ccc43)c2)cc1 ; c1ccc(-c2cccc(-n3c4ccccc4c4cc(-c5ccc6c(c5)c5ccccc5n6-c5ccc6c(c5)oc5ccccc56)ccc43)c2)cc1 ; c1ccc(-c2cccc(-n3c4ccccc4c4cc(-c5ccc6c(c5)c5ccccc5n6-c5cccc6c5oc5ccccc56)ccc43)c2)cc1]

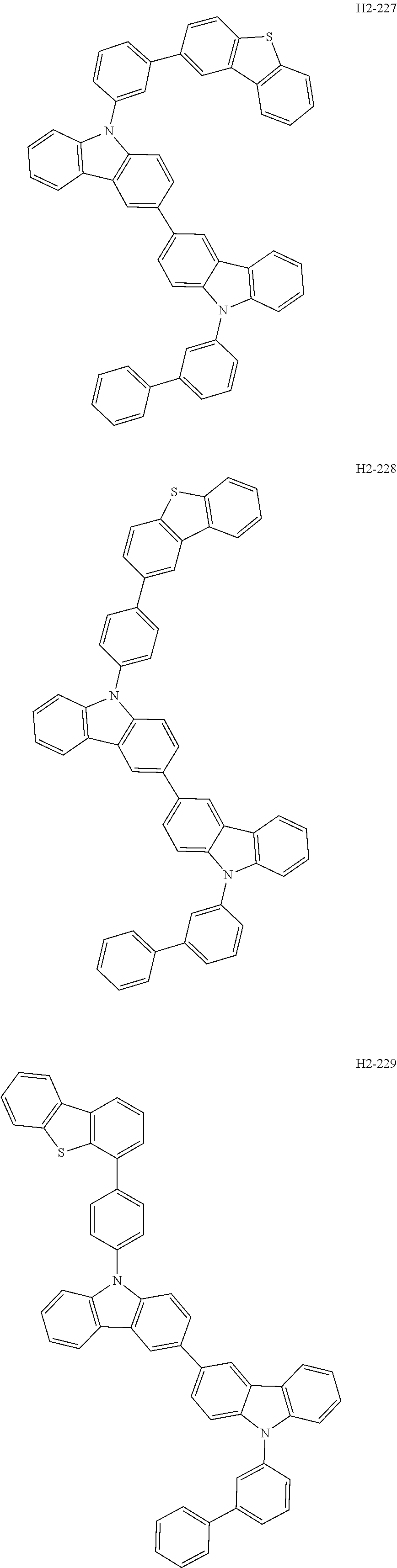 [SMILES: c1ccc(-c2cccc(-n3c4ccccc4c4cc(-c5ccc6c(c5)c5ccccc5n6-c5cccc(-c6ccc7sc8ccccc8c7c6)c5)ccc43)c2)cc1 ; c1ccc(-c2cccc(-n3c4ccccc4c4cc(-c5ccc6c(c5)c5ccccc5n6-c5ccc(-c6ccc7sc8ccccc8c7c6)cc5)ccc43)c2)cc1 ; c1ccc(-c2cccc(-n3c4ccccc4c4cc(-c5ccc6c(c5)c5ccccc5n6-c5ccc(-c6cccc7c6sc6ccccc67)cc5)ccc43)c2)cc1]

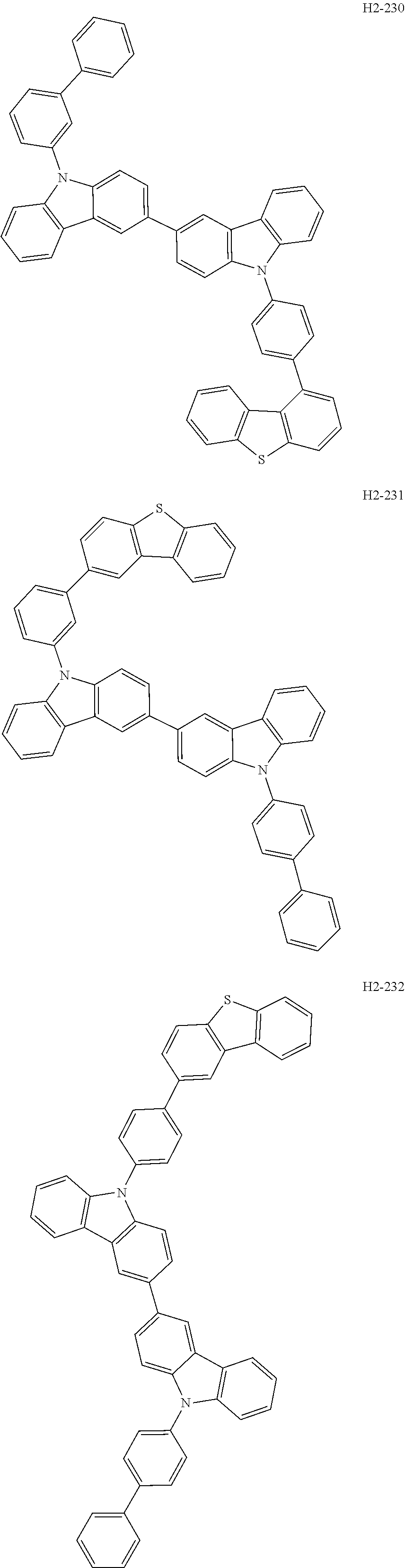 [SMILES: c1ccc(-c2cccc(-n3c4ccccc4c4cc(-c5ccc6c(c5)c5ccccc5n6-c5ccc(-c6cccc7sc8ccccc8c67)cc5)ccc43)c2)cc1 ; c1ccc(-c2ccc(-n3c4ccccc4c4cc(-c5ccc6c(c5)c5ccccc5n6-c5cccc(-c6ccc7sc8ccccc8c7c6)c5)ccc43)cc2)cc1 ; c1ccc(-c2ccc(-n3c4ccccc4c4cc(-c5ccc6c(c5)c5ccccc5n6-c5ccc(-c6ccc7sc8ccccc8c7c6)cc5)ccc43)cc2)cc1]

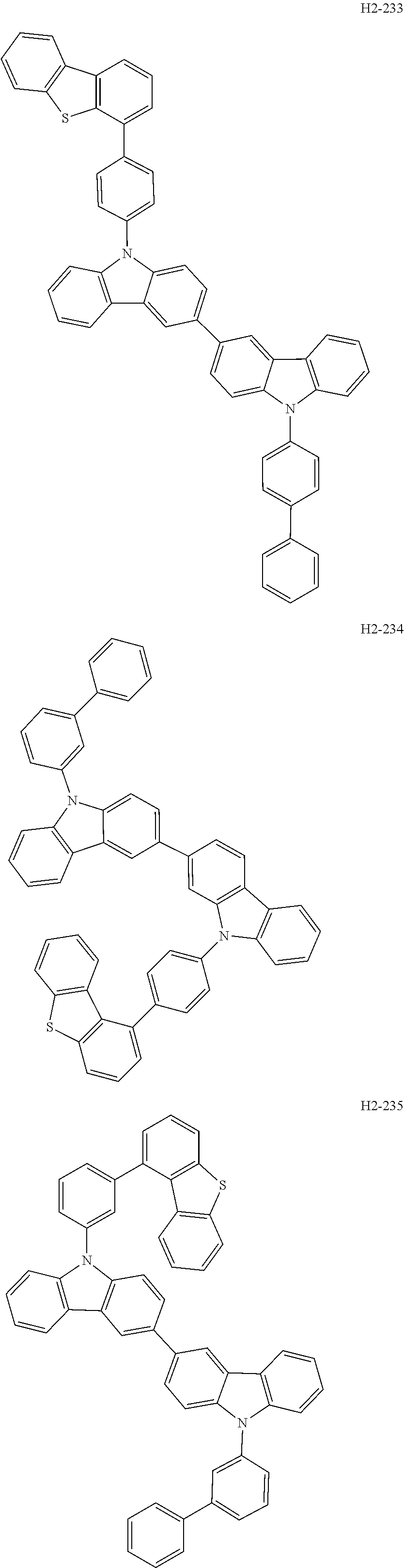 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2cccc(-n3c4ccccc4c4cc(-c5ccc6c7ccccc7n(-c7ccc(-c8cccc9sc%10ccccc%10c89)cc7)c6c5)ccc43)c2)cc1 ; c1ccc(-c2cccc(-n3c4ccccc4c4cc(-c5ccc6c(c5)c5ccccc5n6-c5cccc(-c6cccc7sc8ccccc8c67)c5)ccc43)c2)cc1]

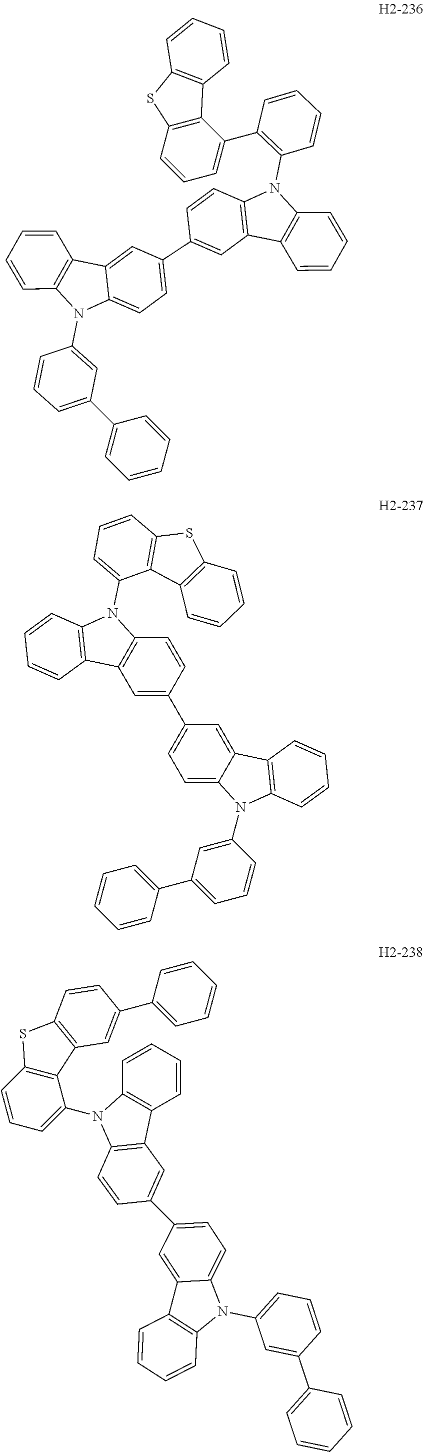

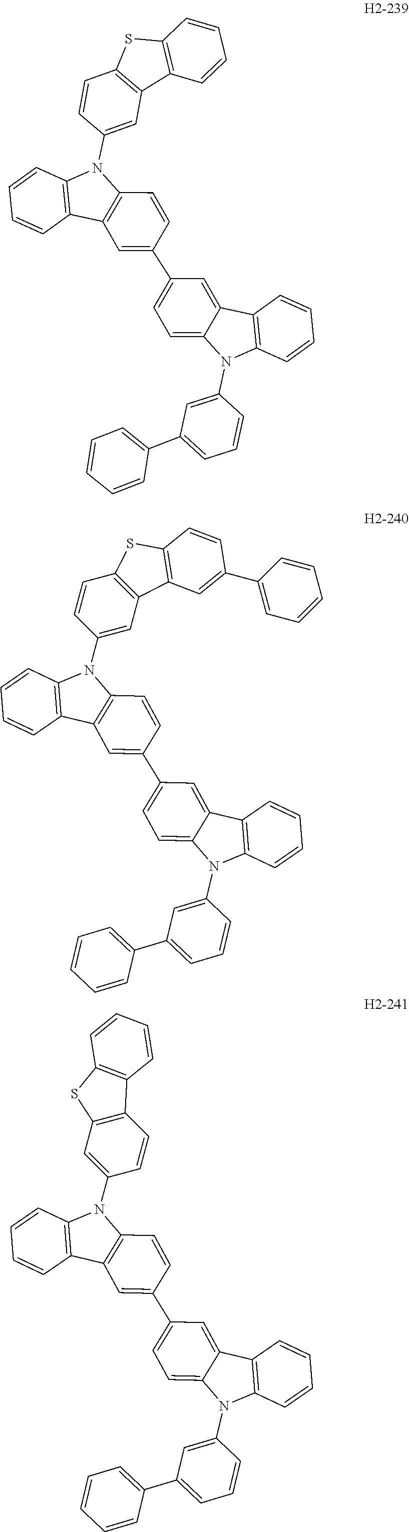

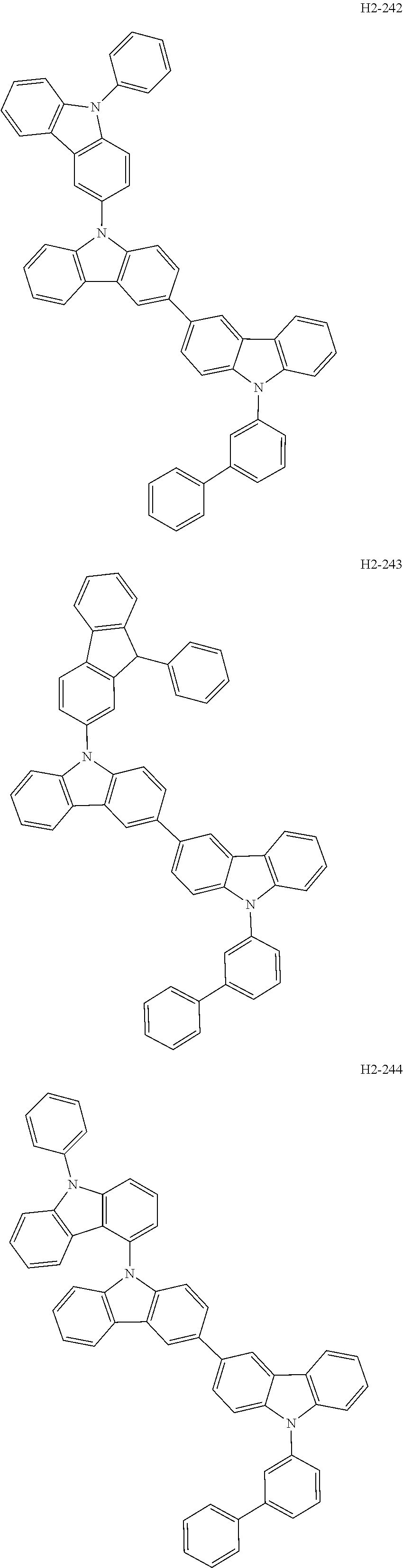 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; c1ccc(-c2cccc(-n3c4ccccc4c4cc(-c5ccc6c(c5)c5ccccc5n6-c5ccc6c(c5)C(c5ccccc5)c5ccccc5-6)ccc43)c2)cc1 ; c1ccc(-c2cccc(-n3c4ccccc4c4cc(-c5ccc6c(c5)c5ccccc5n6-c5cccc6c5c5ccccc5n6-c5ccccc5)ccc43)c2)cc1]

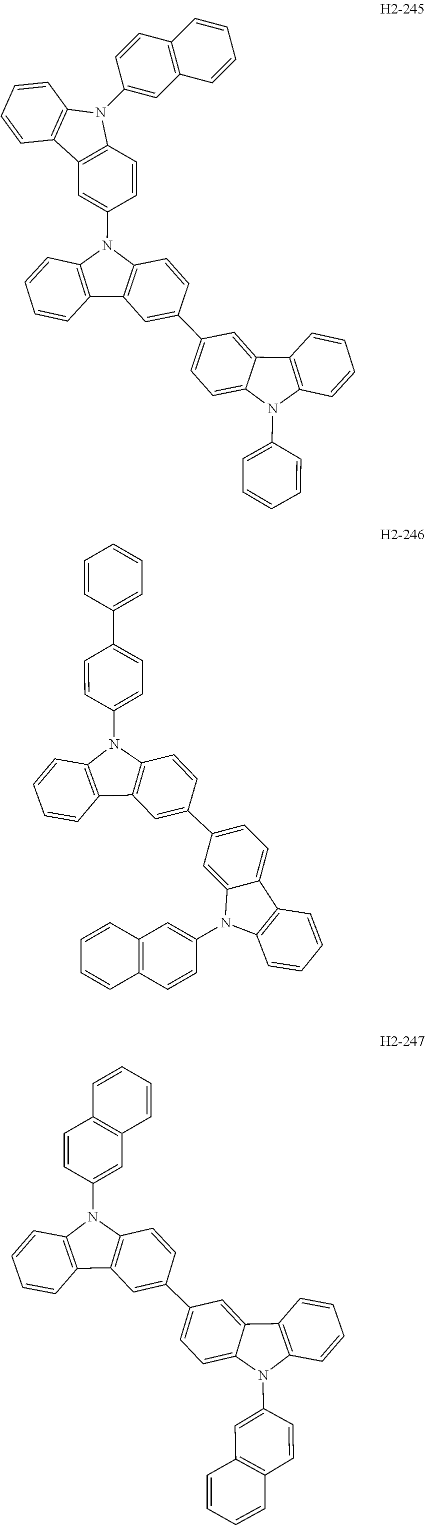 [SMILES: c1ccc(-n2c3ccccc3c3cc(-c4ccc5c(c4)c4ccccc4n5-c4ccc5c(c4)c4ccccc4n5-c4ccc5ccccc5c4)ccc32)cc1 ; c1ccc(-c2ccc(-n3c4ccccc4c4cc(-c5ccc6c7ccccc7n(-c7ccc8ccccc8c7)c6c5)ccc43)cc2)cc1 ; c1ccc2cc(-n3c4ccccc4c4cc(-c5ccc6c(c5)c5ccccc5n6-c5ccc6ccccc6c5)ccc43)ccc2c1]

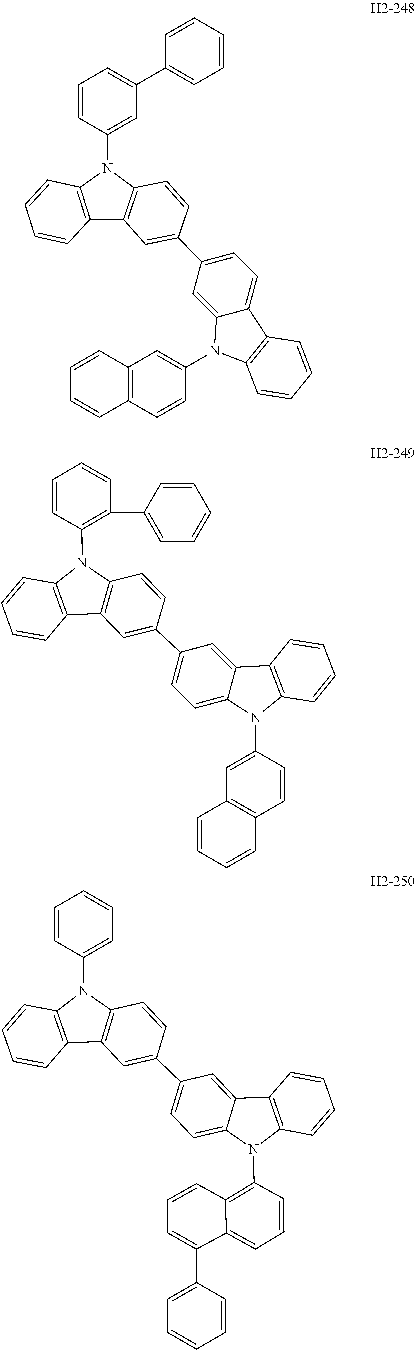 [SMILES: c1ccc(-c2cccc(-n3c4ccccc4c4cc(-c5ccc6c7ccccc7n(-c7ccc8ccccc8c7)c6c5)ccc43)c2)cc1 ; c1ccc(-c2ccccc2-n2c3ccccc3c3cc(-c4ccc5c(c4)c4ccccc4n5-c4ccc5ccccc5c4)ccc32)cc1 ; c1ccc(-c2cccc3c(-n4c5ccccc5c5cc(-c6ccc7c(c6)c6ccccc6n7-c6ccccc6)ccc54)cccc23)cc1]

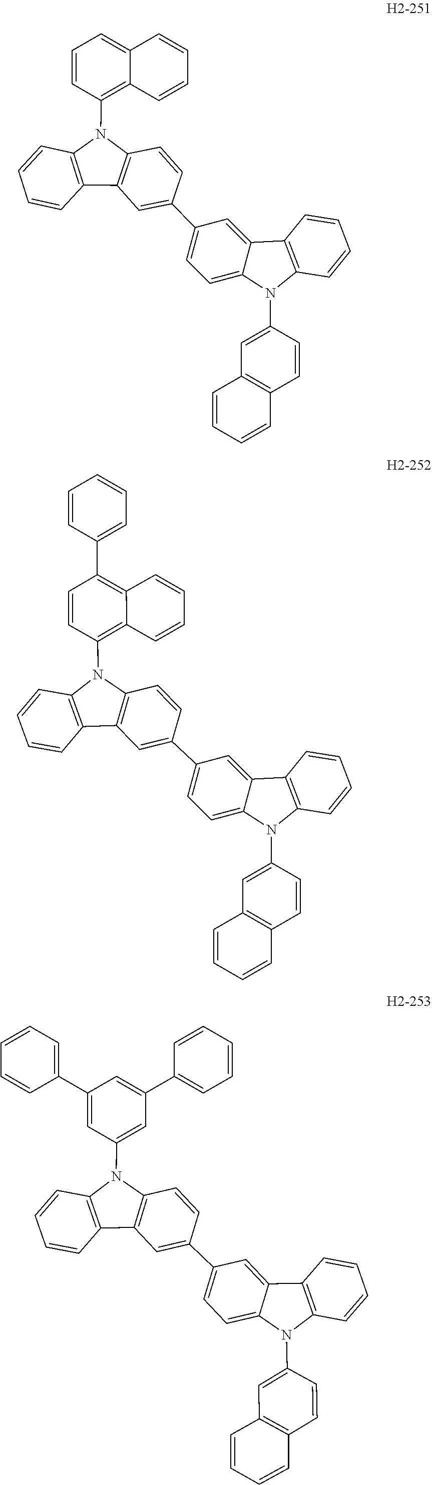 [SMILES: c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5ccc6ccccc6c5)ccc43)c3ccccc23)cc1 ; c1ccc(-c2cc(-c3ccccc3)cc(-n3c4ccccc4c4cc(-c5ccc6c(c5)c5ccccc5n6-c5ccc6ccccc6c5)ccc43)c2)cc1]

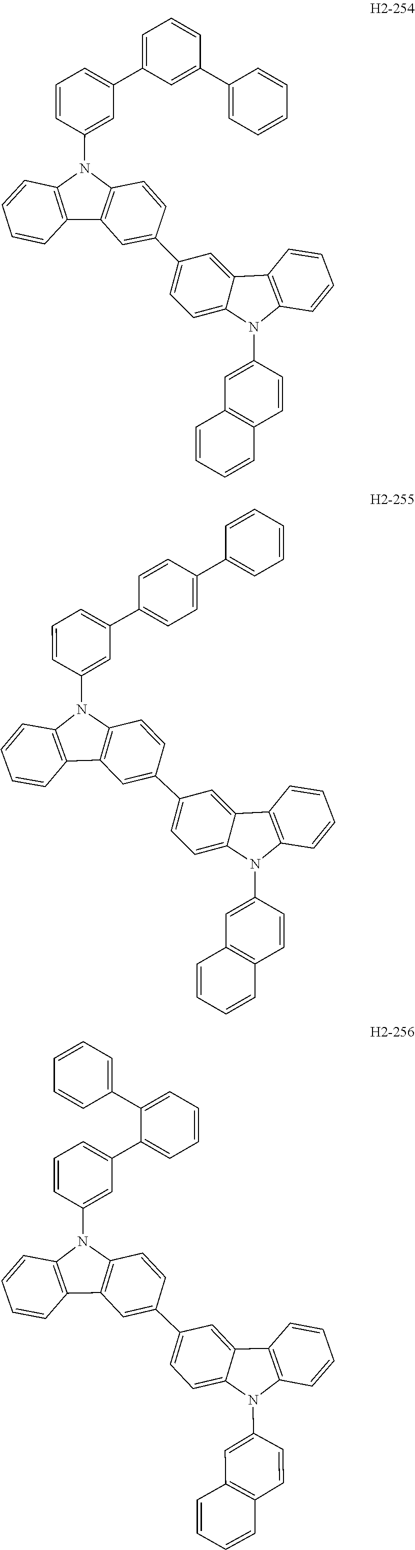

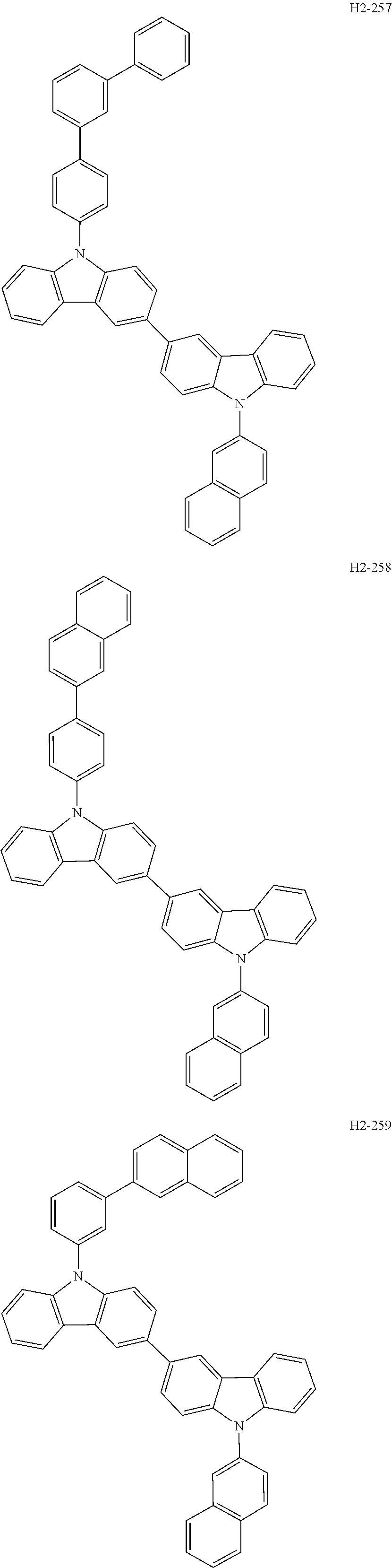

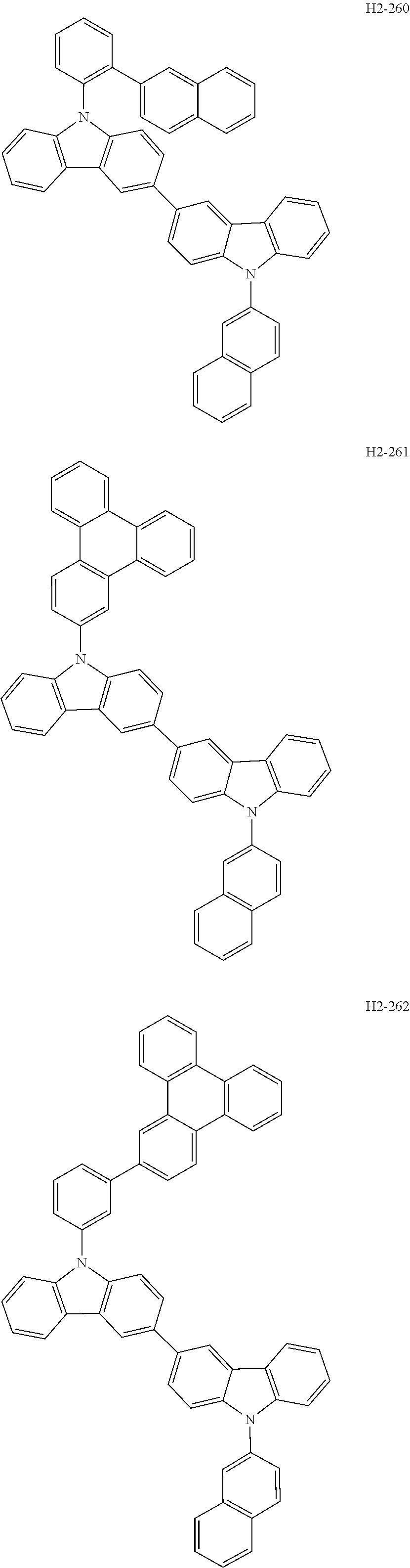 [SMILES: c1ccc(-n2c3ccccc3c3cc(-c4ccc5c(c4)c4ccccc4n5-c4ccc5ccccc5c4)ccc32)c(-c2ccc3ccccc3c2)c1 ; c1ccc2cc(-n3c4ccccc4c4cc(-c5ccc6c(c5)c5ccccc5n6-c5ccc6c7ccccc7c7ccccc7c6c5)ccc43)ccc2c1 ; c1cc(-c2ccc3c4ccccc4c4ccccc4c3c2)cc(-n2c3ccccc3c3cc(-c4ccc5c(c4)c4ccccc4n5-c4ccc5ccccc5c4)ccc32)c1]

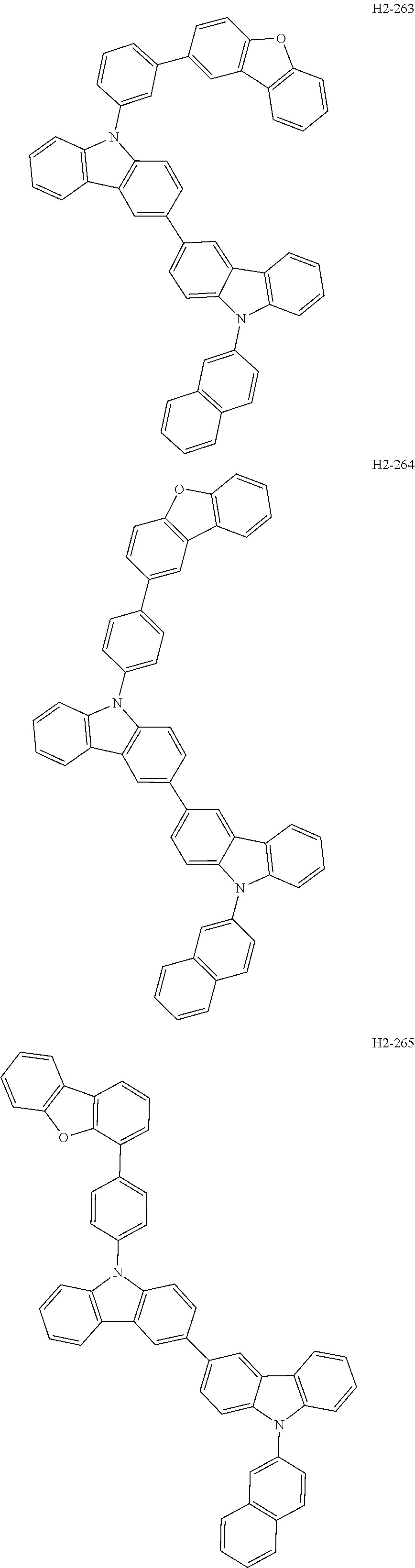

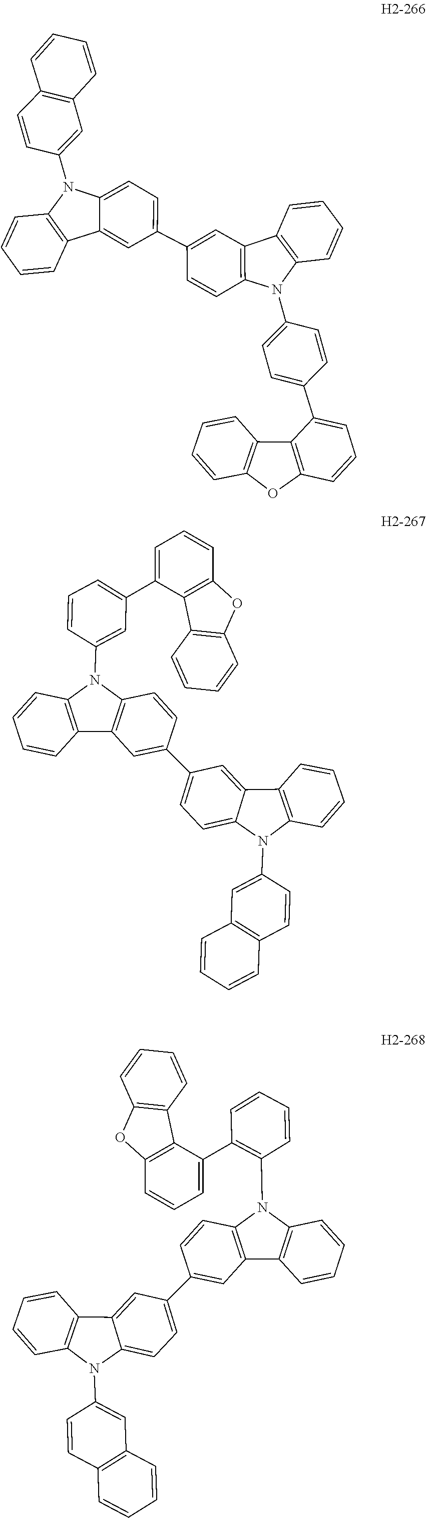 [SMILES: c1ccc2cc(-n3c4ccccc4c4cc(-c5ccc6c(c5)c5ccccc5n6-c5ccc(-c6cccc7oc8ccccc8c67)cc5)ccc43)ccc2c1 ; c1cc(-c2cccc3oc4ccccc4c23)cc(-n2c3ccccc3c3cc(-c4ccc5c(c4)c4ccccc4n5-c4ccc5ccccc5c4)ccc32)c1 ; c1ccc(-n2c3ccccc3c3cc(-c4ccc5c(c4)c4ccccc4n5-c4ccc5ccccc5c4)ccc32)c(-c2cccc3oc4ccccc4c23)c1]

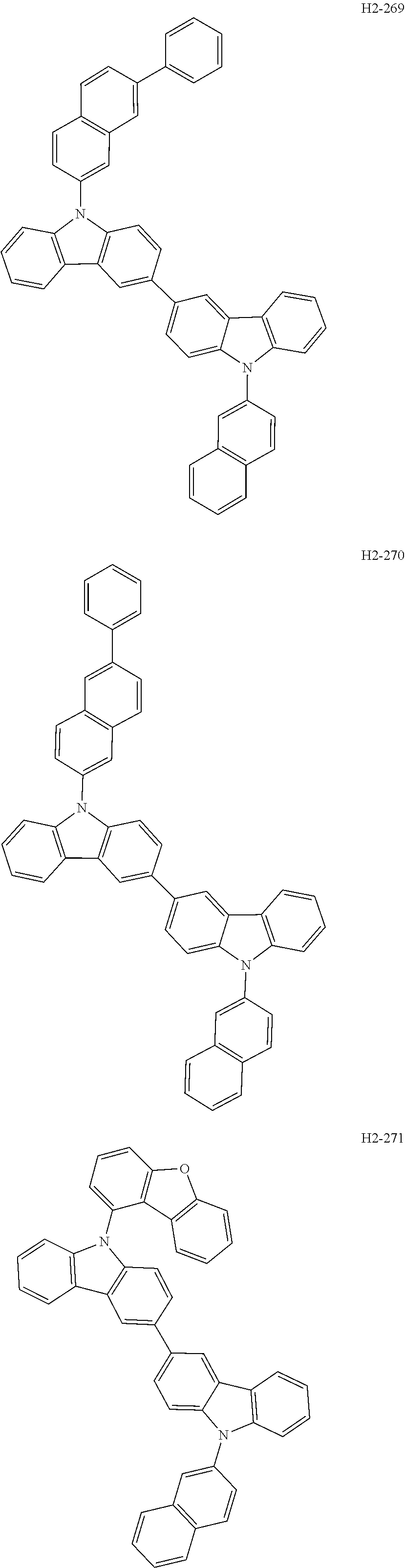 [SMILES: c1ccc(-c2ccc3ccc(-n4c5ccccc5c5cc(-c6ccc7c(c6)c6ccccc6n7-c6ccc7ccccc7c6)ccc54)cc3c2)cc1 ; c1ccc(-c2ccc3cc(-n4c5ccccc5c5cc(-c6ccc7c(c6)c6ccccc6n7-c6ccc7ccccc7c6)ccc54)ccc3c2)cc1 ; c1ccc2cc(-n3c4ccccc4c4cc(-c5ccc6c(c5)c5ccccc5n6-c5cccc6oc7ccccc7c56)ccc43)ccc2c1]

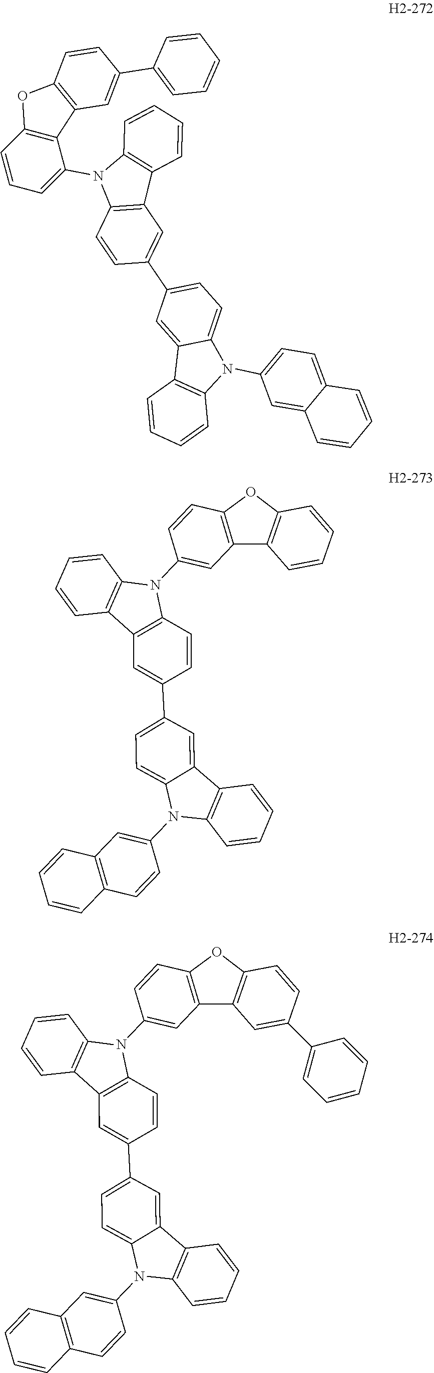 [SMILES: c1ccc(-c2ccc3oc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6oc7ccccc7c6c5)ccc43)ccc2c1 ; c1ccc(-c2ccc3oc4ccc(-n5c6ccccc6c6cc(-c7ccc8c(c7)c7ccccc7n8-c7ccc8ccccc8c7)ccc65)cc4c3c2)cc1]

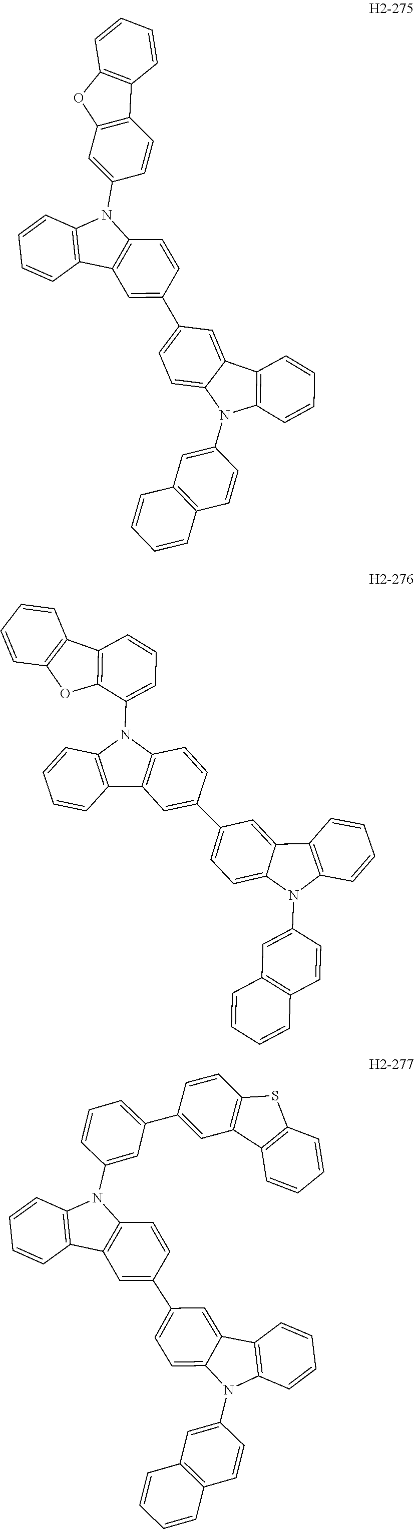 [SMILES: c1ccc2cc(-n3c4ccccc4c4cc(-c5ccc6c(c5)c5ccccc5n6-c5ccc6c(c5)oc5ccccc56)ccc43)ccc2c1 ; c1ccc2cc(-n3c4ccccc4c4cc(-c5ccc6c(c5)c5ccccc5n6-c5cccc6c5oc5ccccc56)ccc43)ccc2c1 ; c1cc(-c2ccc3sc4ccccc4c3c2)cc(-n2c3ccccc3c3cc(-c4ccc5c(c4)c4ccccc4n5-c4ccc5ccccc5c4)ccc32)c1]

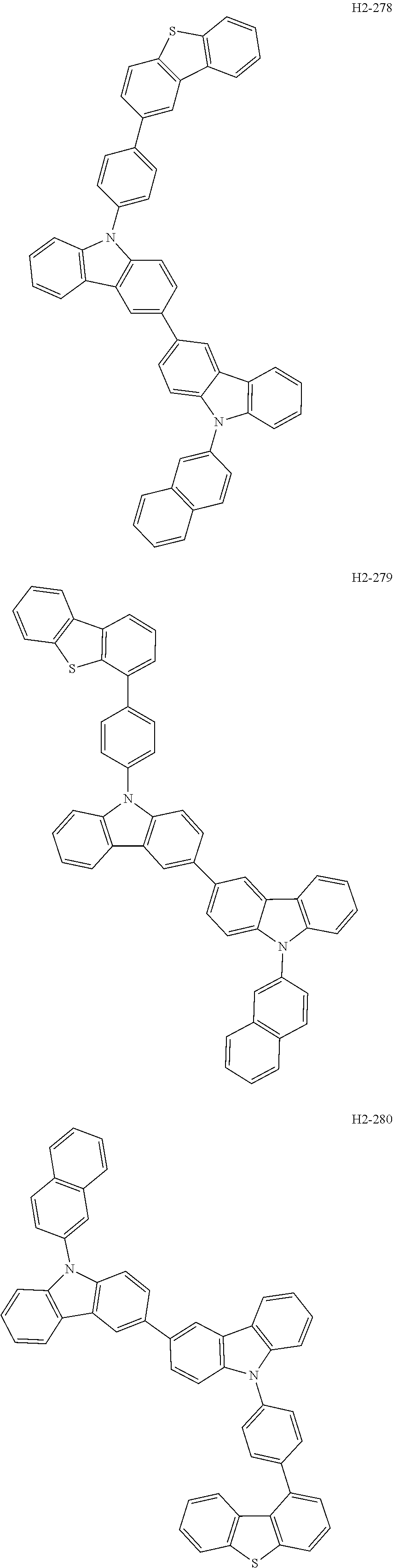 [SMILES: c1ccc2cc(-n3c4ccccc4c4cc(-c5ccc6c(c5)c5ccccc5n6-c5ccc(-c6ccc7sc8ccccc8c7c6)cc5)ccc43)ccc2c1 ; c1ccc2cc(-n3c4ccccc4c4cc(-c5ccc6c(c5)c5ccccc5n6-c5ccc(-c6cccc7c6sc6ccccc67)cc5)ccc43)ccc2c1 ; c1ccc2cc(-n3c4ccccc4c4cc(-c5ccc6c(c5)c5ccccc5n6-c5ccc(-c6cccc7sc8ccccc8c67)cc5)ccc43)ccc2c1]

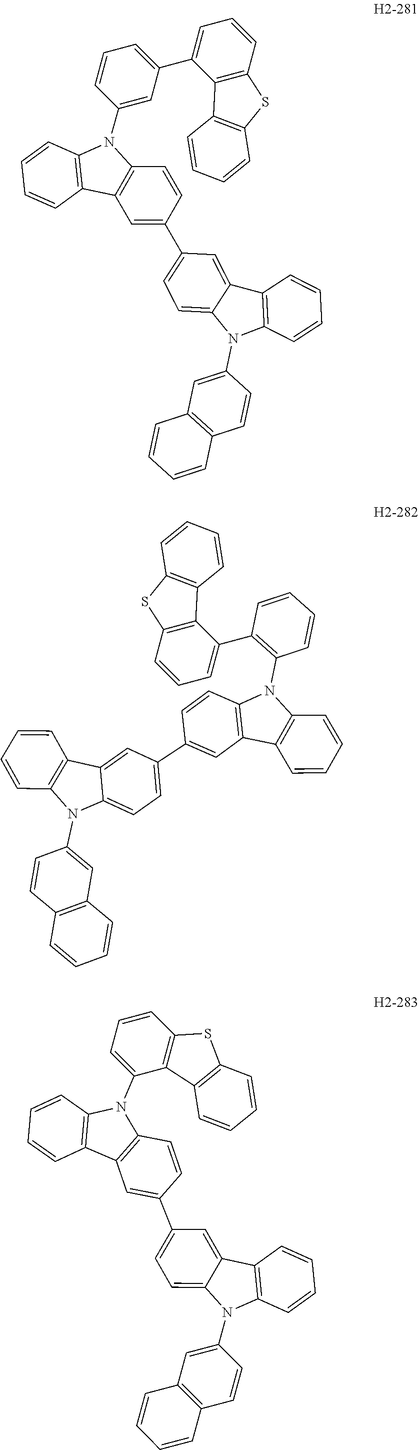

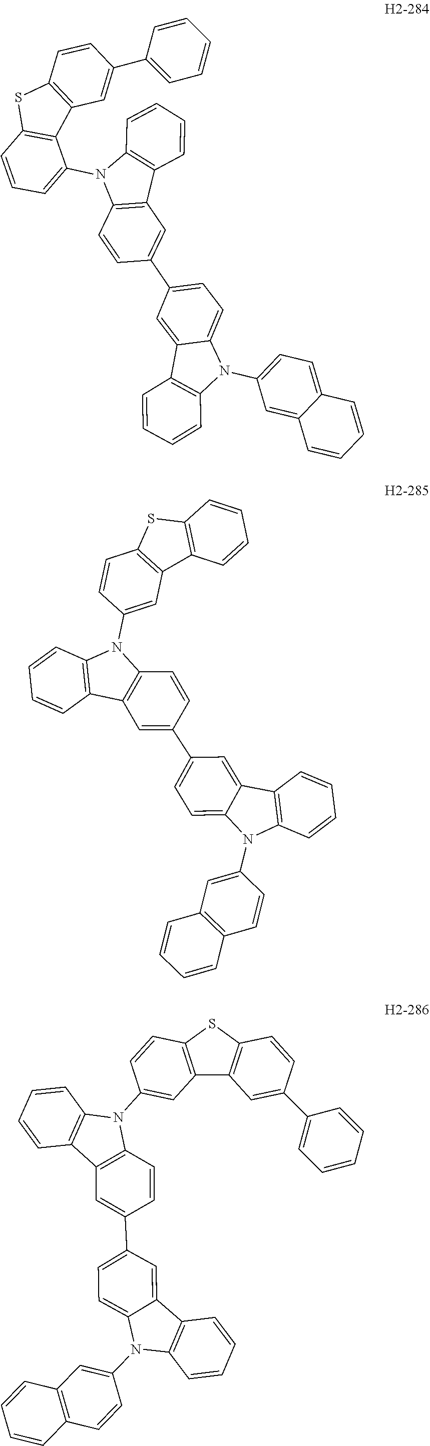 [SMILES: c1ccc(-c2ccc3sc4cccc(-n5c6ccccc6c6cc(-c7ccc8c(c7)c7ccccc7n8-c7ccc8ccccc8c7)ccc65)c4c3c2)cc1 ; c1ccc2cc(-n3c4ccccc4c4cc(-c5ccc6c(c5)c5ccccc5n6-c5ccc6sc7ccccc7c6c5)ccc43)ccc2c1 ; c1ccc(-c2ccc3sc4ccc(-n5c6ccccc6c6cc(-c7ccc8c(c7)c7ccccc7n8-c7ccc8ccccc8c7)ccc65)cc4c3c2)cc1]

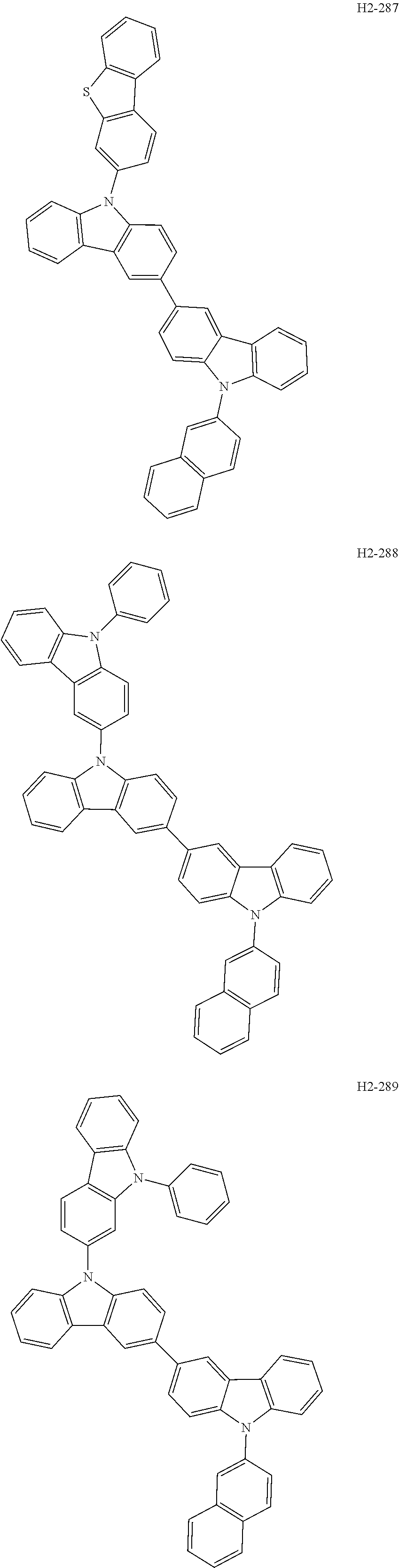 [SMILES: c1ccc2cc(-n3c4ccccc4c4cc(-c5ccc6c(c5)c5ccccc5n6-c5ccc6c(c5)sc5ccccc56)ccc43)ccc2c1 ; c1ccc(-n2c3ccccc3c3cc(-n4c5ccccc5c5cc(-c6ccc7c(c6)c6ccccc6n7-c6ccc7ccccc7c6)ccc54)ccc32)cc1 ; c1ccc(-n2c3ccccc3c3ccc(-n4c5ccccc5c5cc(-c6ccc7c(c6)c6ccccc6n7-c6ccc7ccccc7c6)ccc54)cc32)cc1]

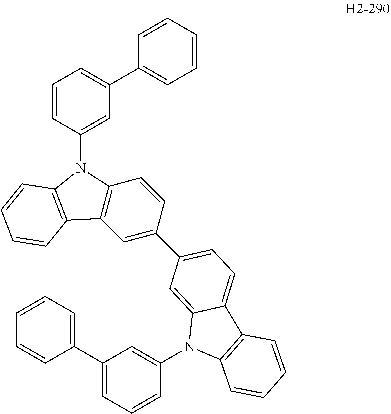 [SMILES: c1ccc(-c2cccc(-n3c4ccccc4c4cc(-c5ccc6c7ccccc7n(-c7cccc(-c8ccccc8)c7)c6c5)ccc43)c2)cc1]

In the above compounds, Dn means that n number of hydrogen atoms are replaced with deuterium.

The combination of at least one of compounds C-1 to C-377 and at least one of compounds H2-1 to H2-290 may be used in an organic electroluminescent device.

The compounds represented by formulas 1-1 and 1 according to the present disclosure may be prepared by a synthetic method known to one skilled in the art, for example, by referring to the following reaction scheme 1, but is not limited thereto.

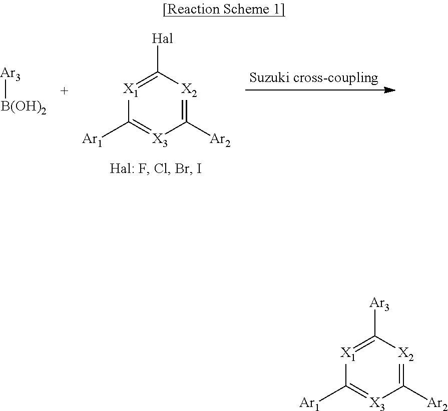

In reaction scheme 1, X₁ to X₃ and Ar₁ to Ar₃ are as defined in formula 1, respectively.

The compound represented by formula 2 according to the present disclosure may be prepared by a synthetic method known to one skilled in the art, for example, by referring to the following reaction scheme 2, but is not limited thereto.

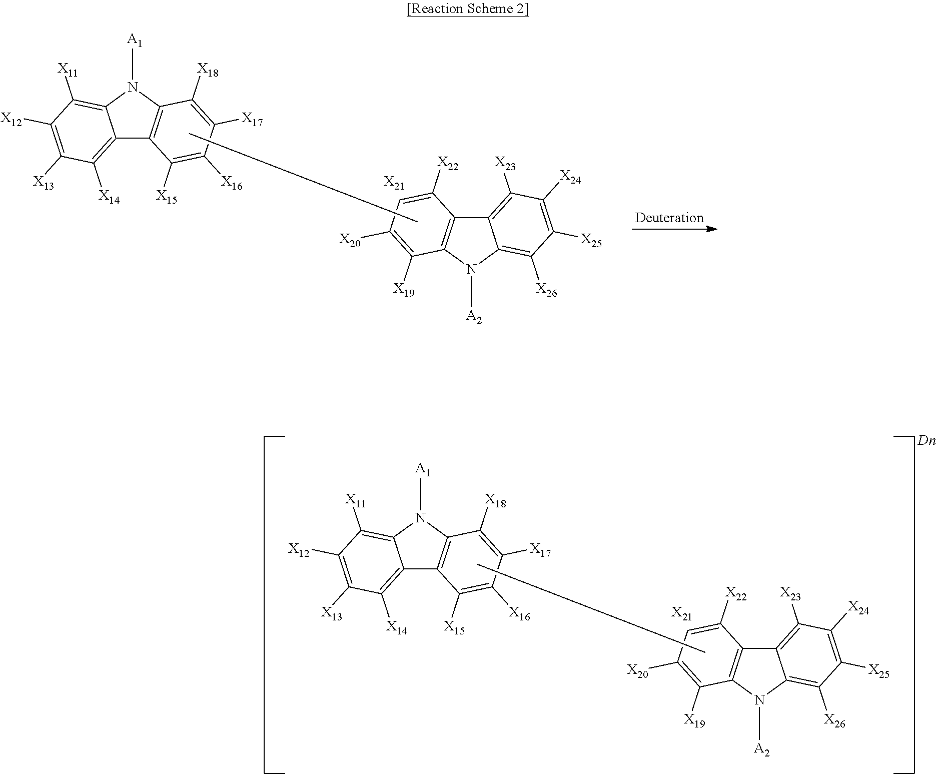

In reaction scheme 2, A₁, A₂, X₁₁ to X₂₆, and n are as defined in formula 2, and Dn means that n number of hydrogen atoms are replaced with deuterium.

Although illustrative synthesis examples of the compound represented by formulas 1-1, 1, and 2 are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formulas 1-1, 1, and 2 but are not specified in the specific synthesis examples, are bonded.

In addition, the deuterated compounds of formulas 1-1, 1, and 2 can be prepared in a similar manner using deuterated precursor materials, or more generally by treating a non-deuterated compound with a deuterated solvent, D6-benzene, in the presence of Lewis acid H/D exchange catalysts such as aluminum trichloride or ethylaluminum chloride. In addition, the degree of deuteration can be controlled by varying reaction conditions such as reaction temperature. For example, the number of deuterium atoms in formulas 1-1, 1, and 2 may be controlled by adjusting the reaction temperature and time, equivalent weight of acid, and the like.

The present disclosure provides an organic electroluminescent device comprising an anode; a cathode; and at least one light-emitting layer between the anode and the cathode, wherein the at least one light-emitting layer comprises the plurality of host materials according to the present disclosure. The first host material and the second host material according to the present disclosure may be included in one light-emitting layer or in respective different light-emitting layers among a plurality of light-emitting layers. The plurality of host materials according to the present disclosure may comprise the compound represented by formula 1 and the compound represented by formula 2 in a ratio of about 1:99 to about 99:1, preferably about 10:90 to about 90:10, and more preferably about 30:70 to about 70:30. In addition, the compound represented by formula 1 and the compound represented by formula 2 may be combined in desired ratios by mixing after putting them in a shaker, by putting them in a glass tube and dissolving them by applying heat and then collecting them, or by dissolving them in a solvent, etc.

According to one embodiment of the present disclosure, the doping concentration of a dopant compound with respect to a host compound in a light-emitting layer may be less than 20 wt %. The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be selected from metallated complex compounds of iridium (Ir), osmium (Os), copper (Cu), and platinum (Pt), preferably selected from ortho-metallated complex compounds of iridium (Ir), osmium (Os), copper (Cu), and platinum (Pt), and more preferably an ortho-metallated iridium complex compound.

The dopant comprised in the organic electroluminescent device of the present disclosure may be a compound represented by the following formula 101, but is not limited thereto.

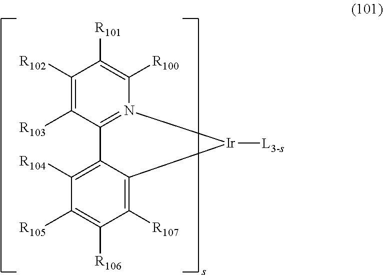

In formula 101,
L is any one selected from the following structures 1 to 3:

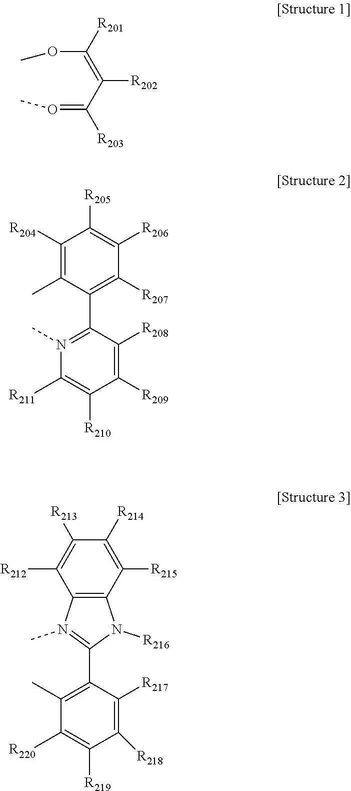

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

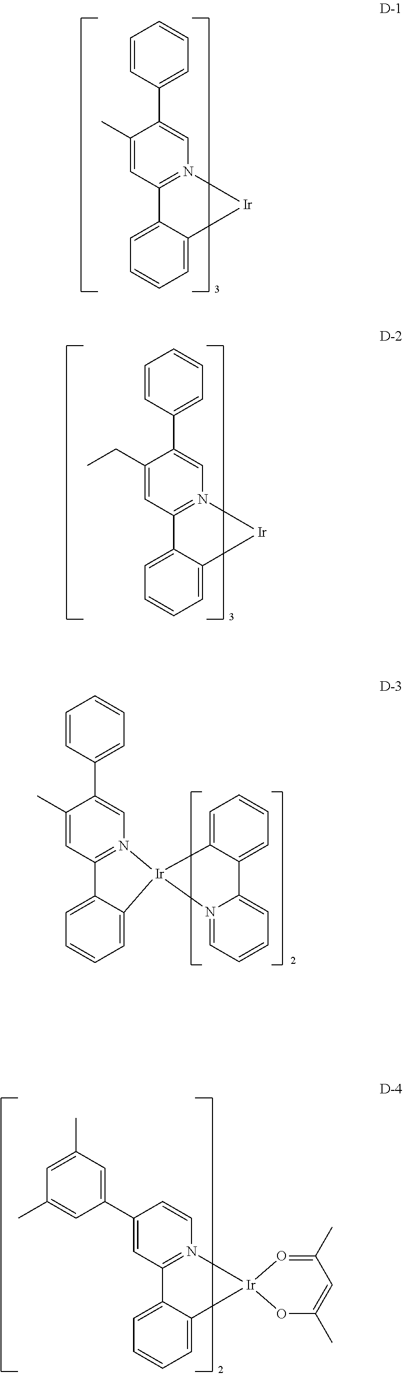

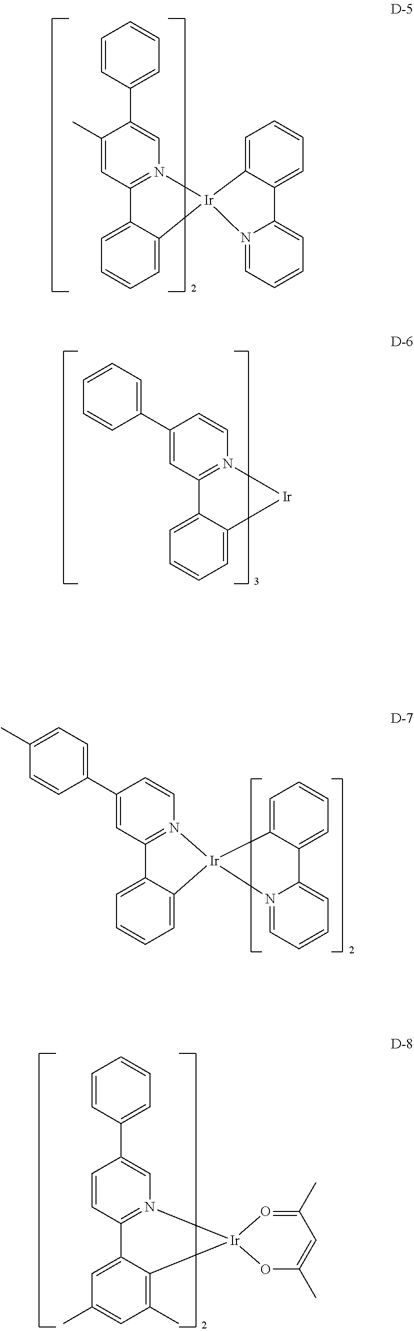

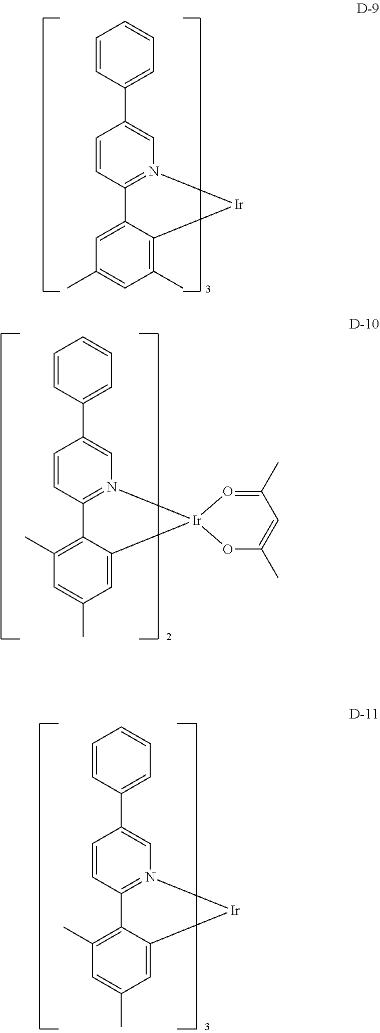

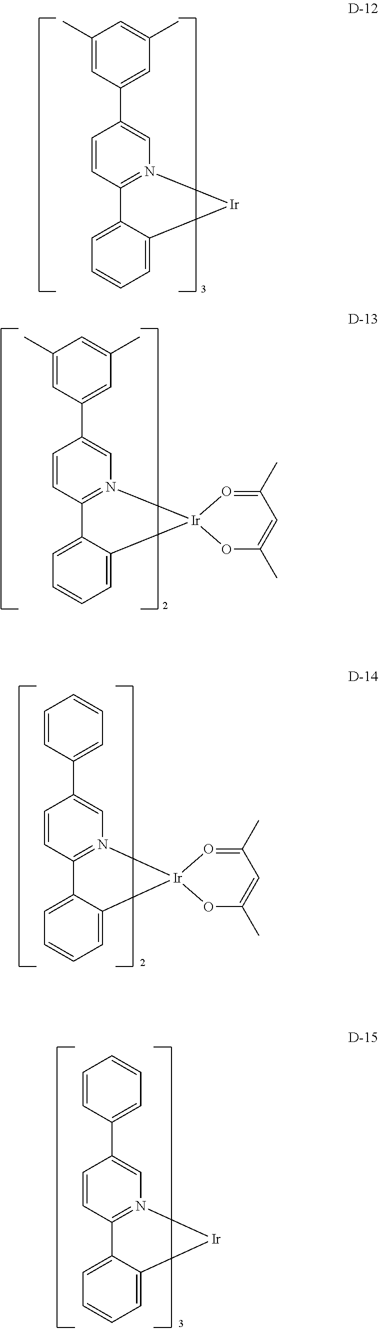

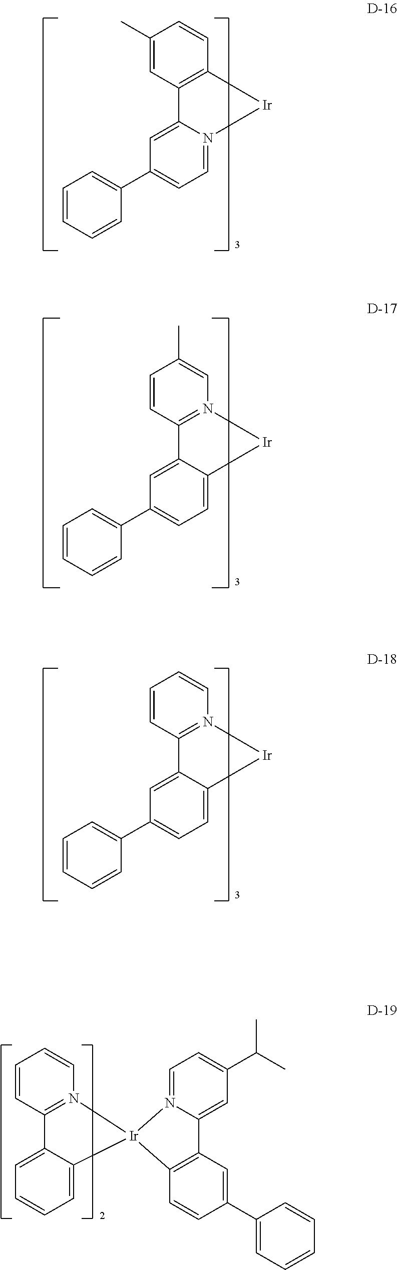

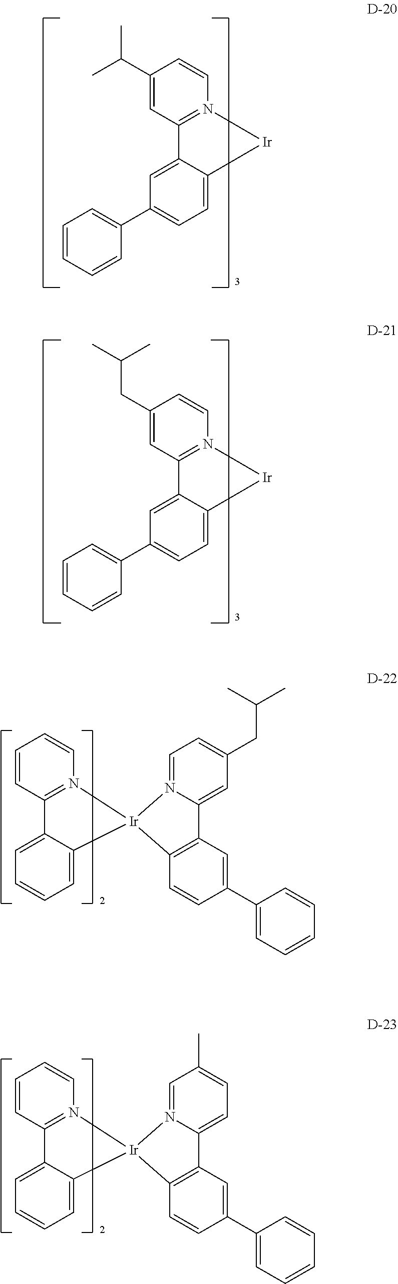

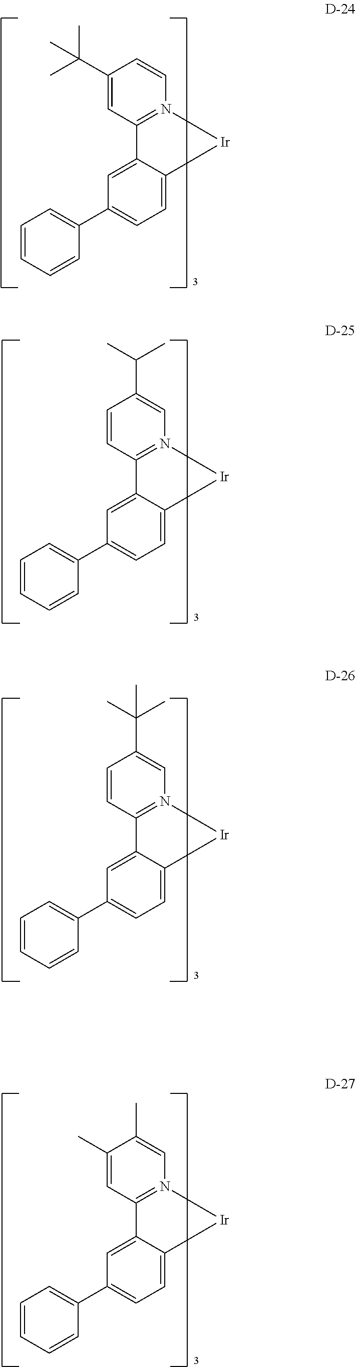

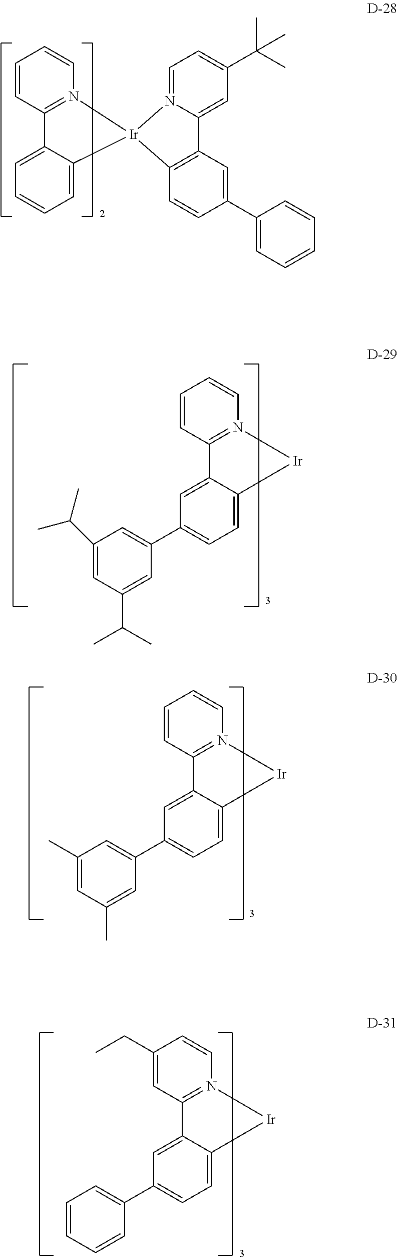

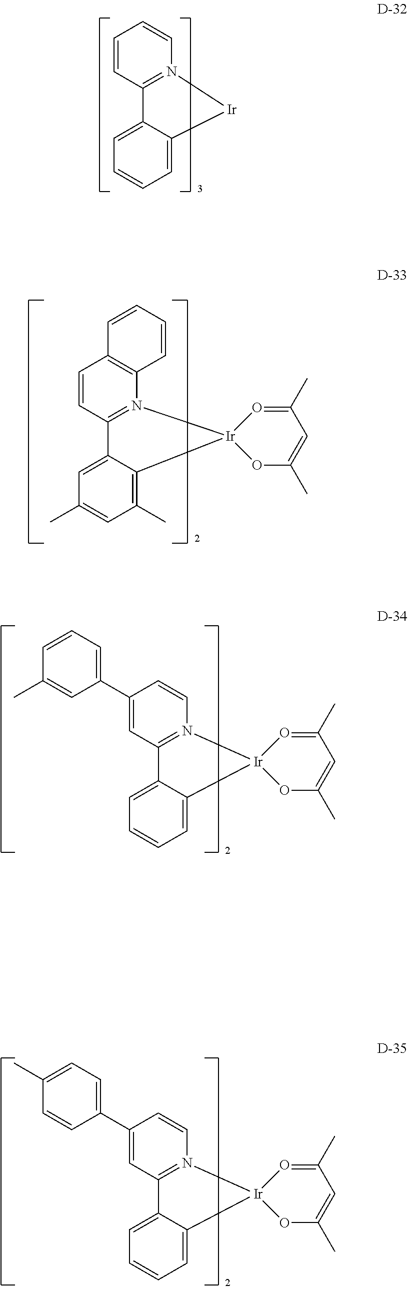

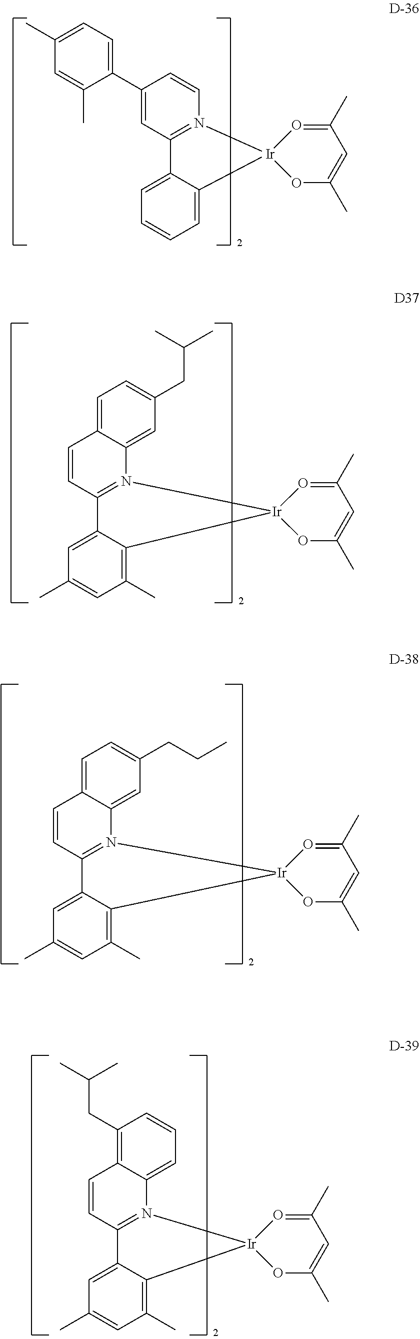

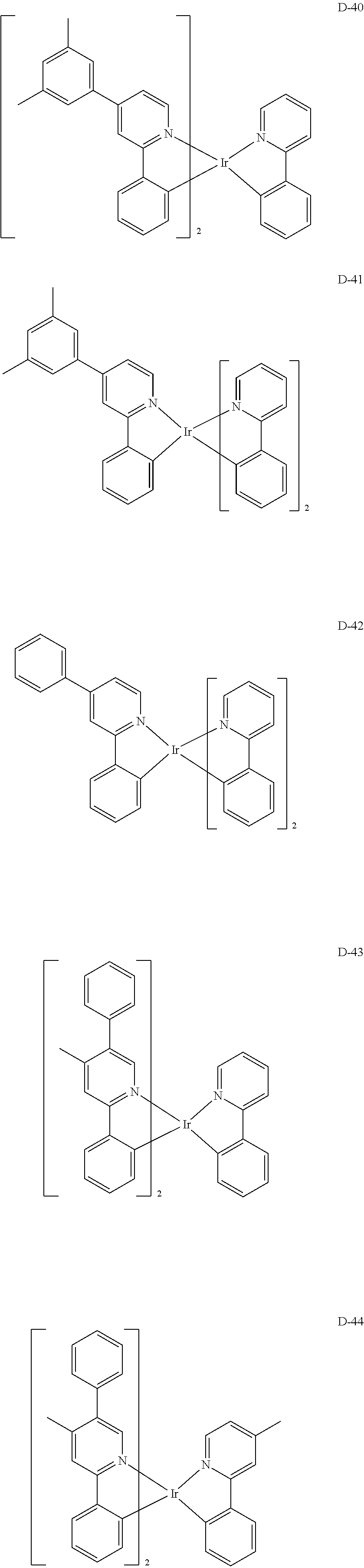

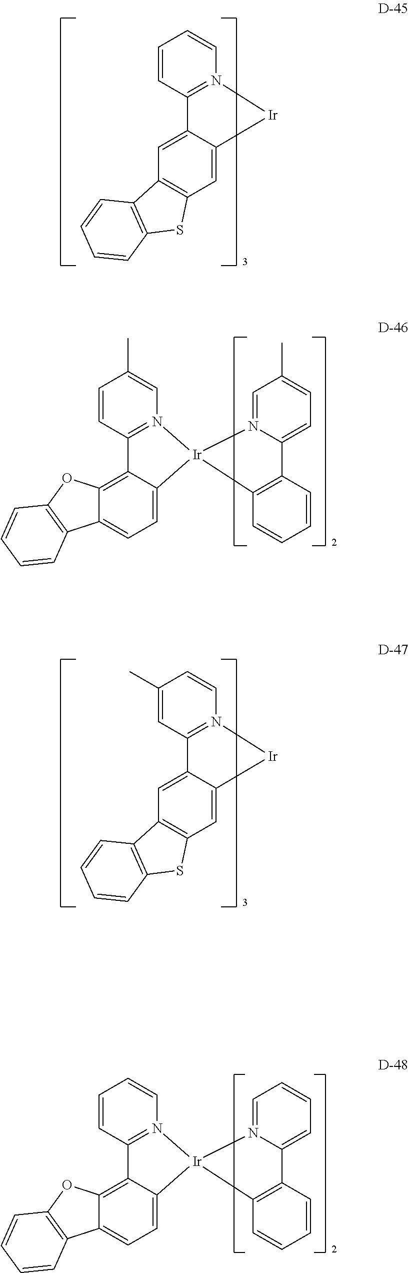

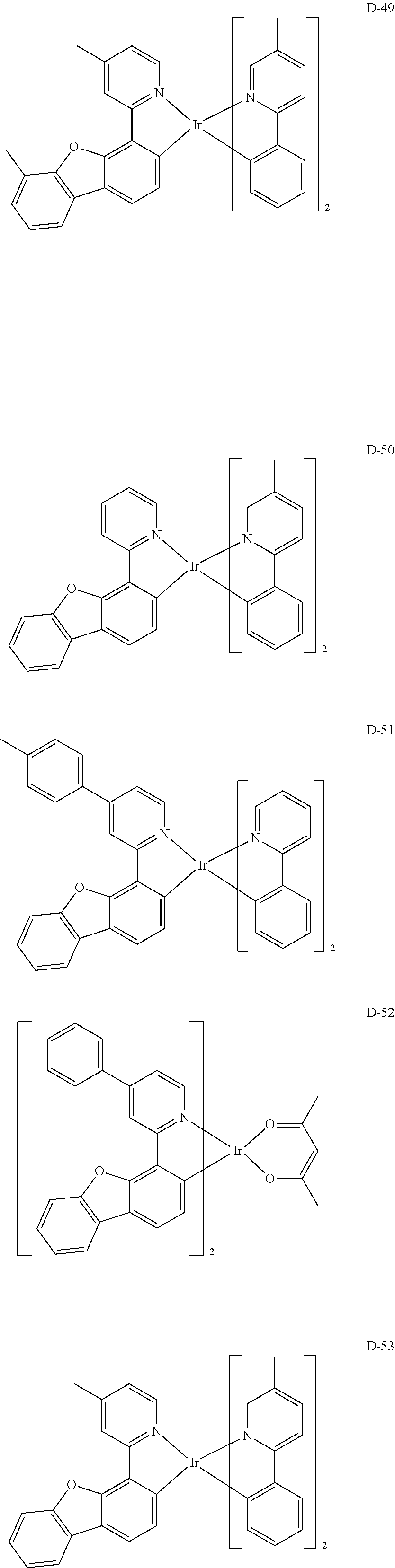

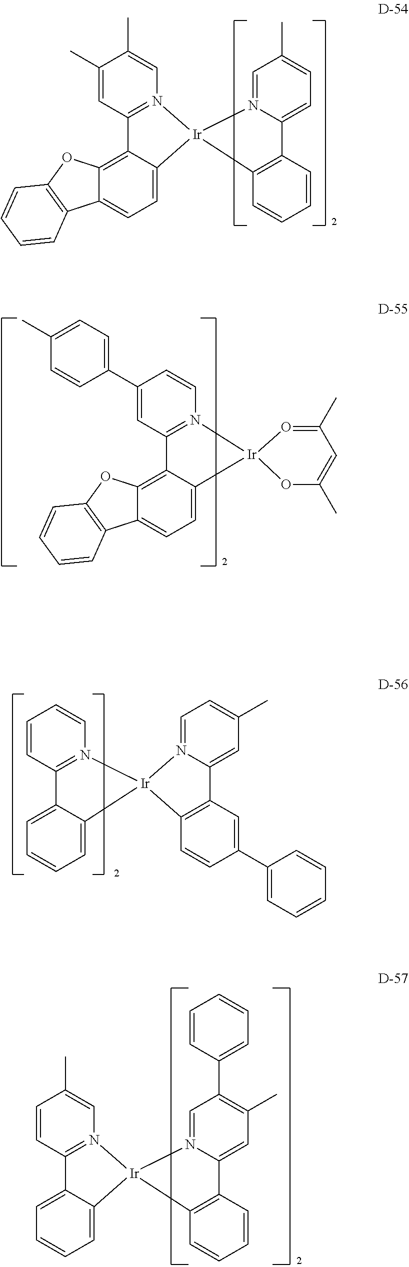

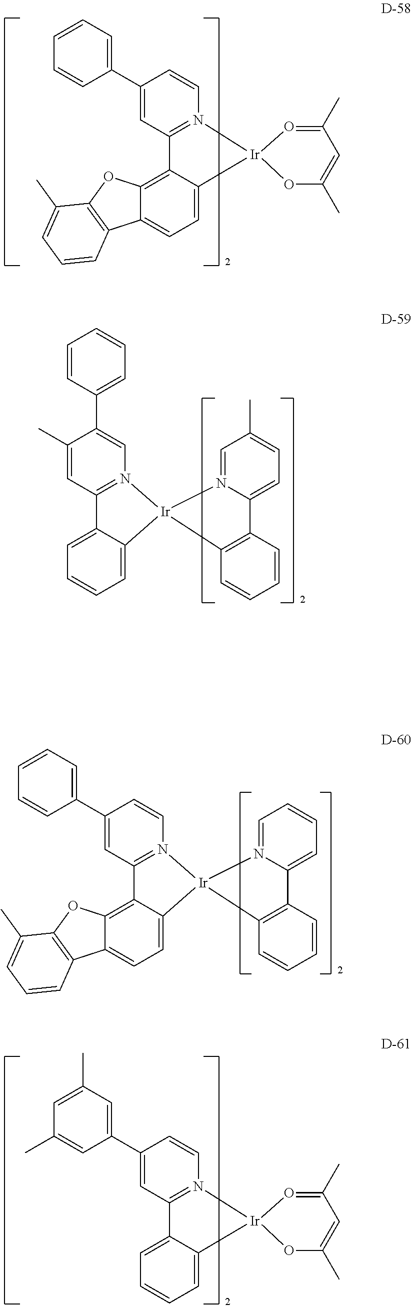

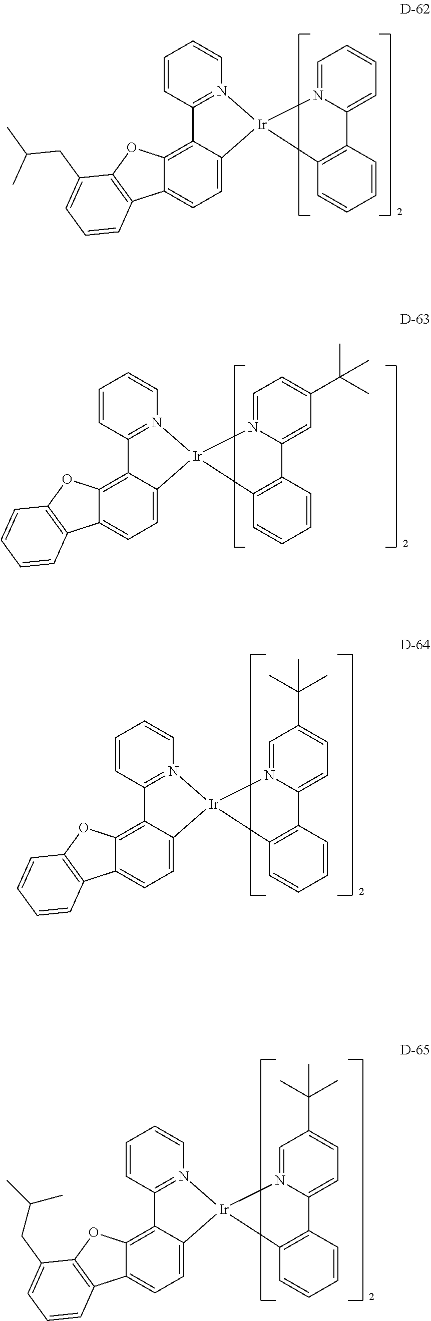

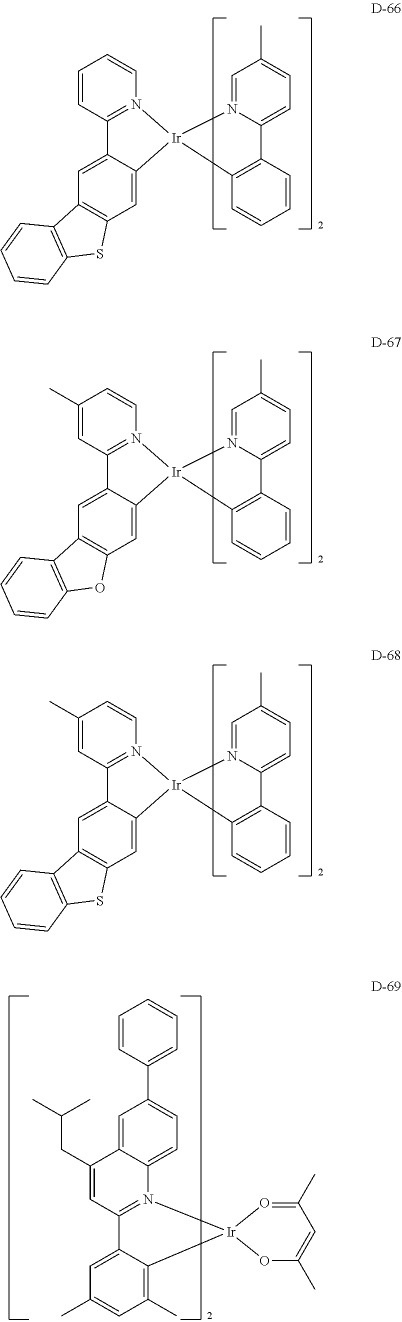

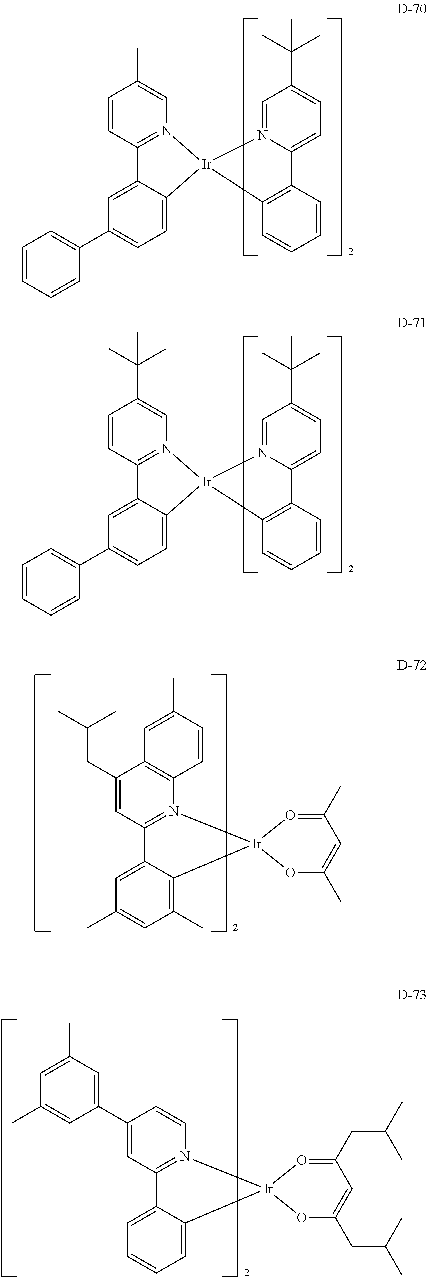

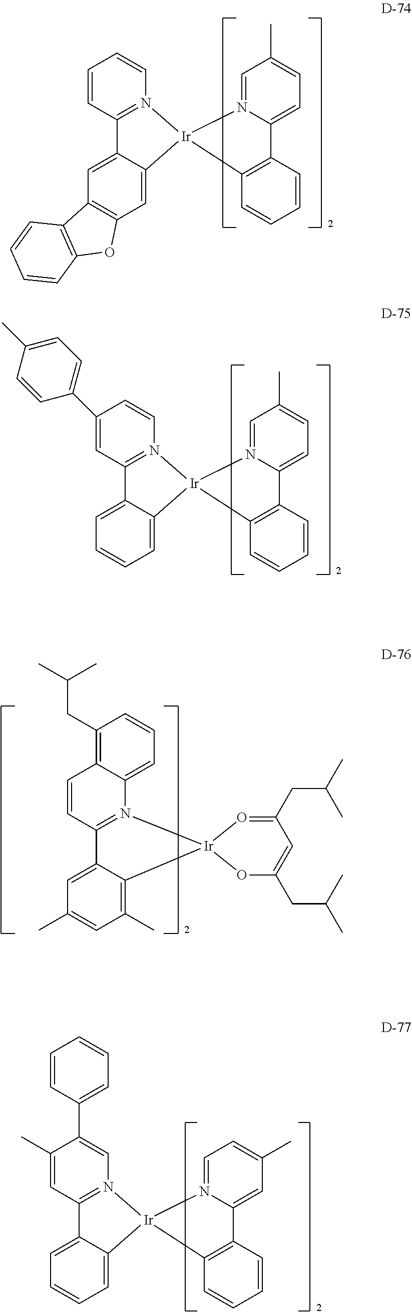

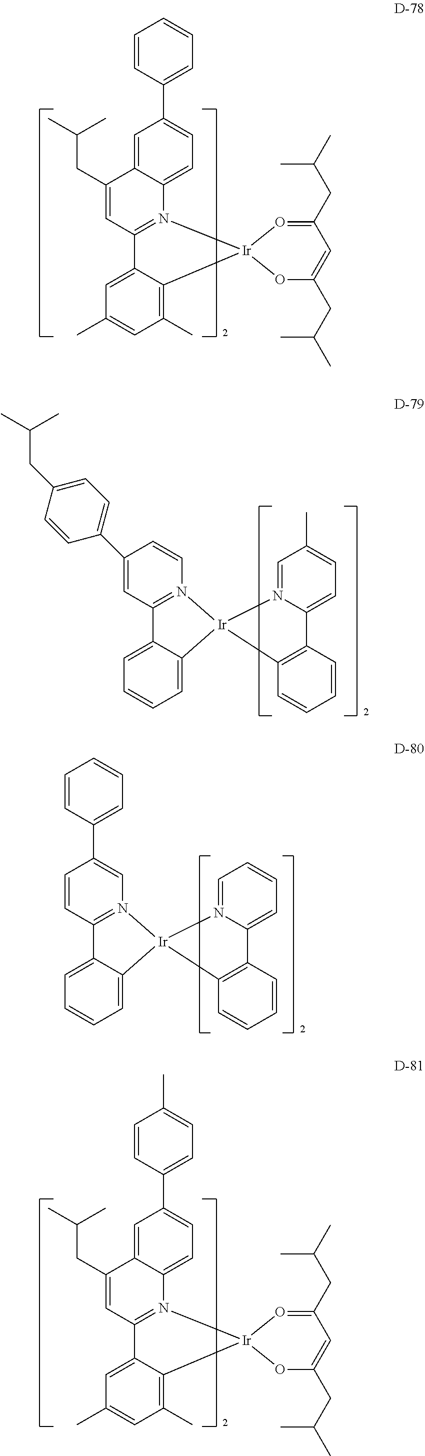

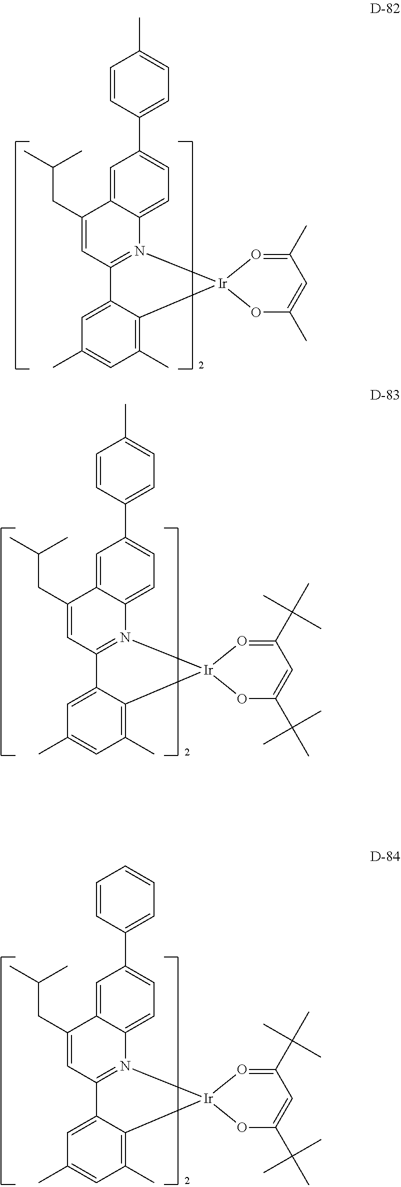

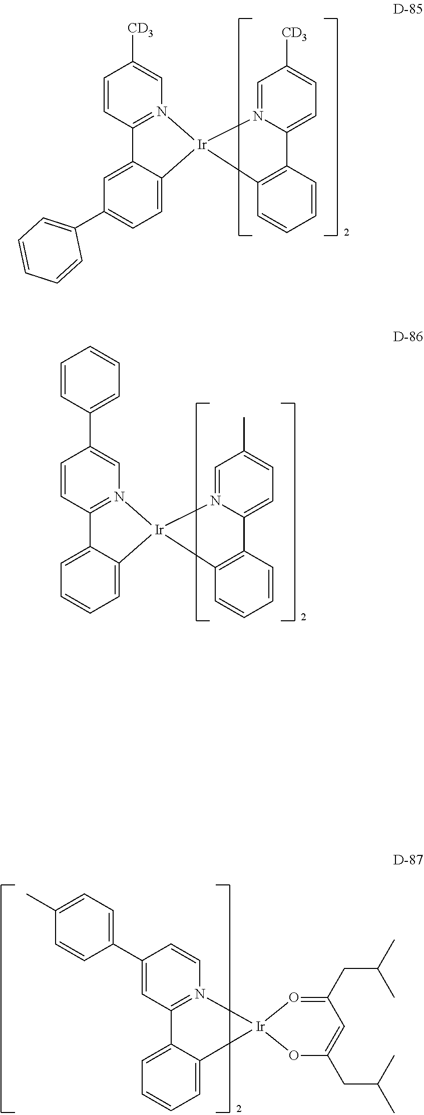

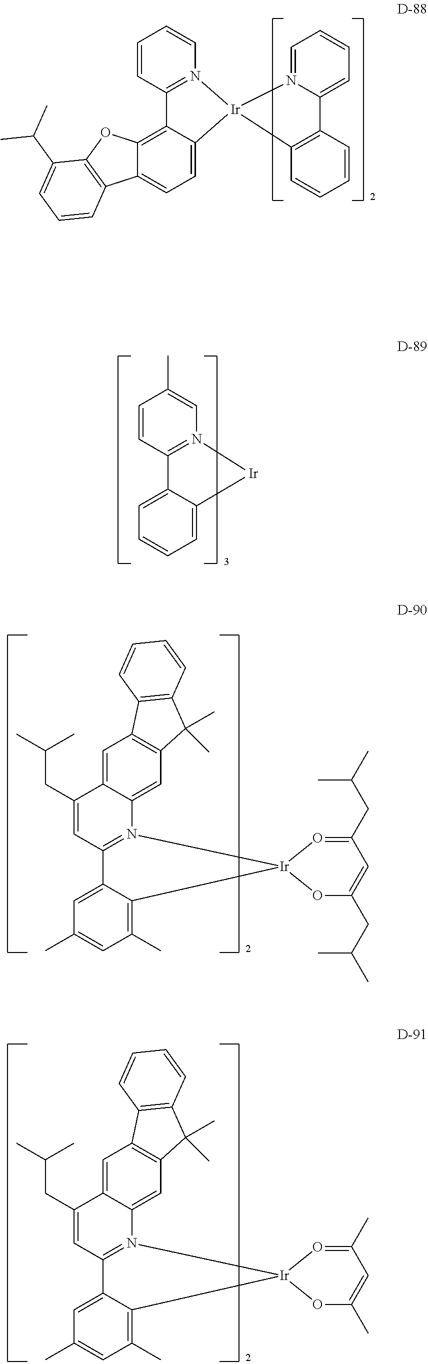

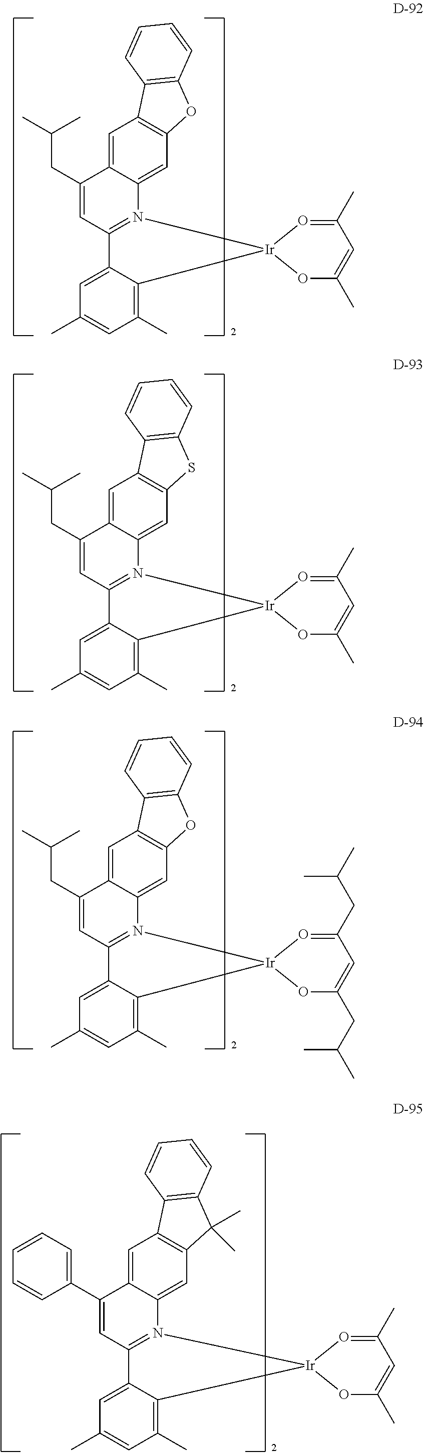

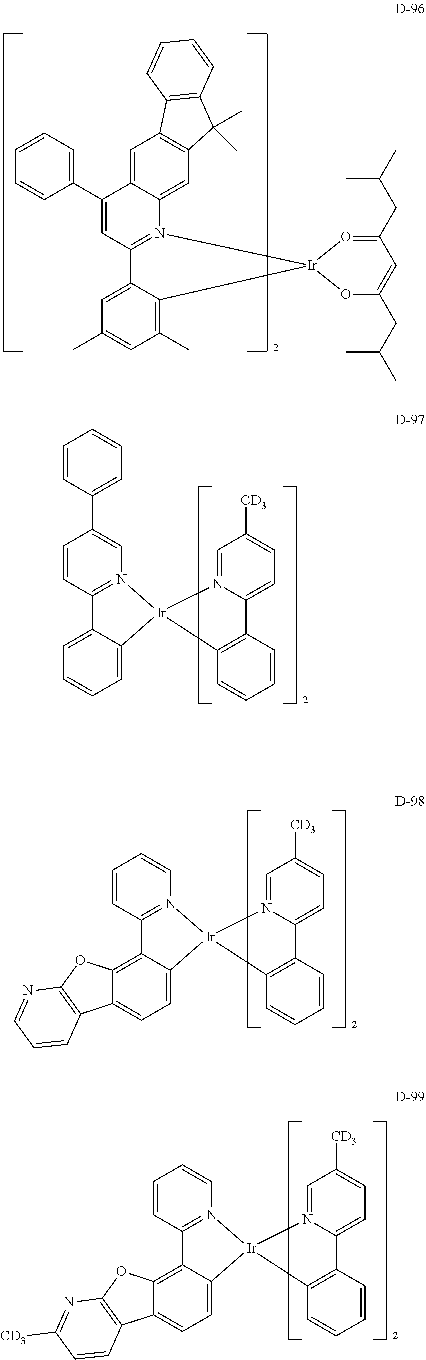

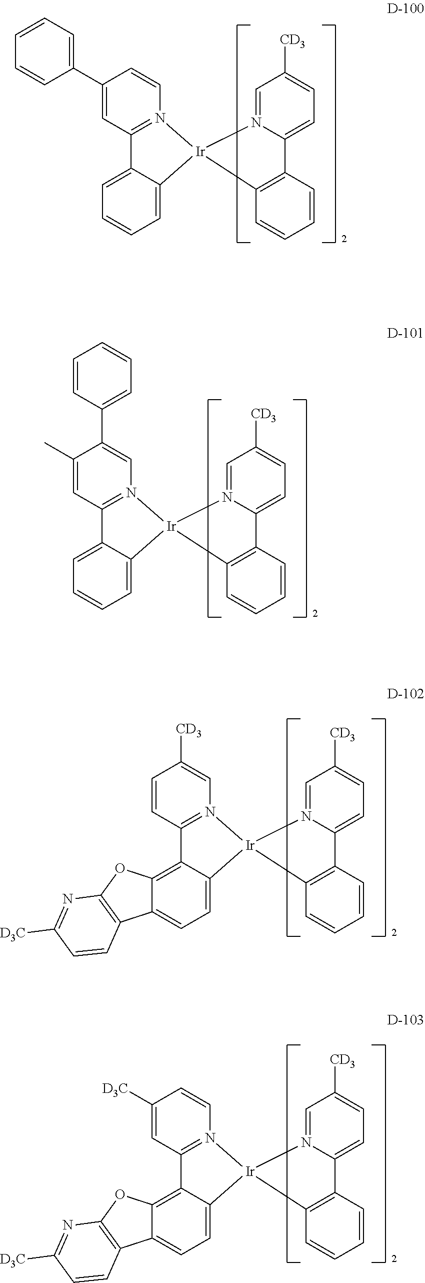

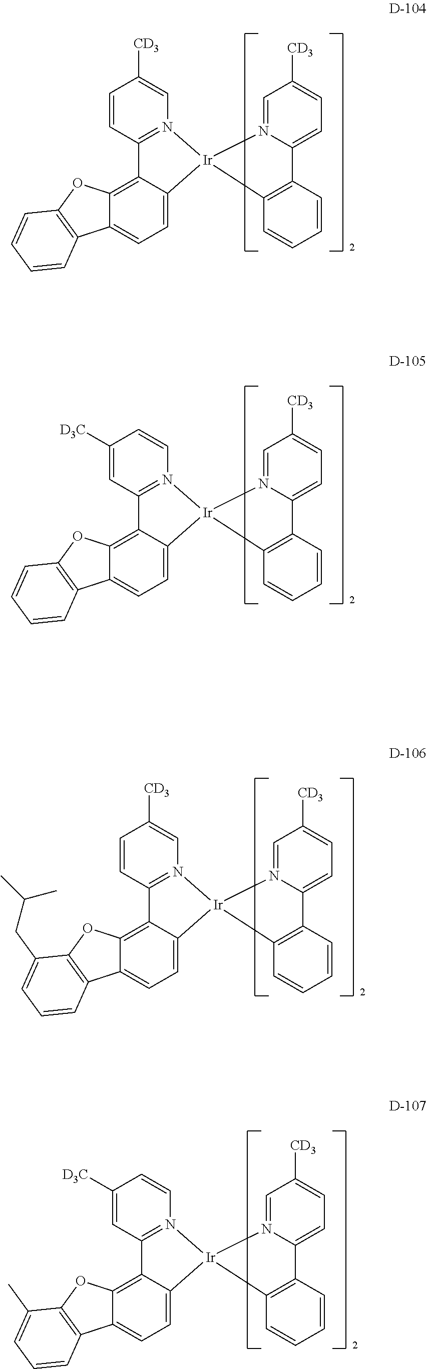

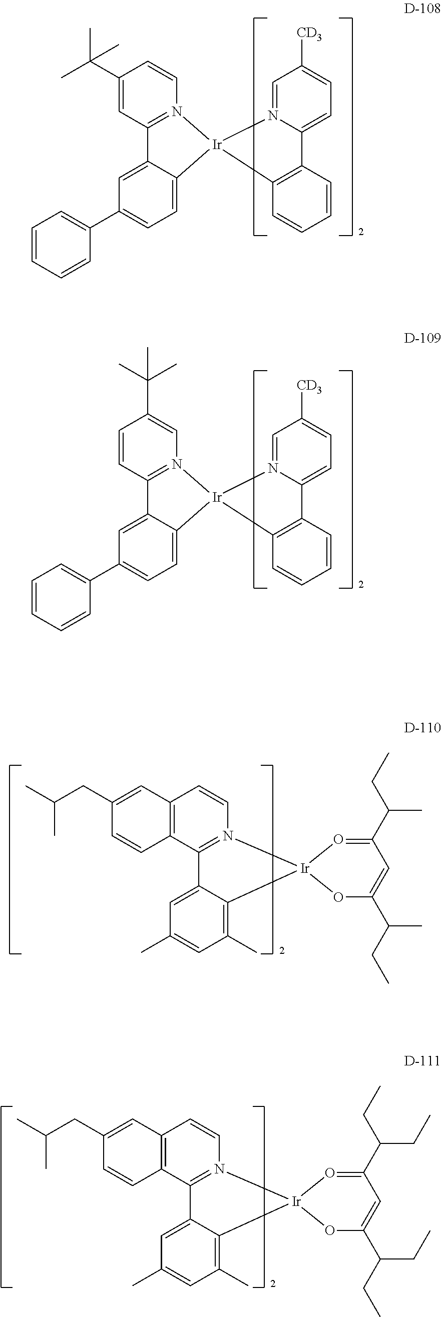

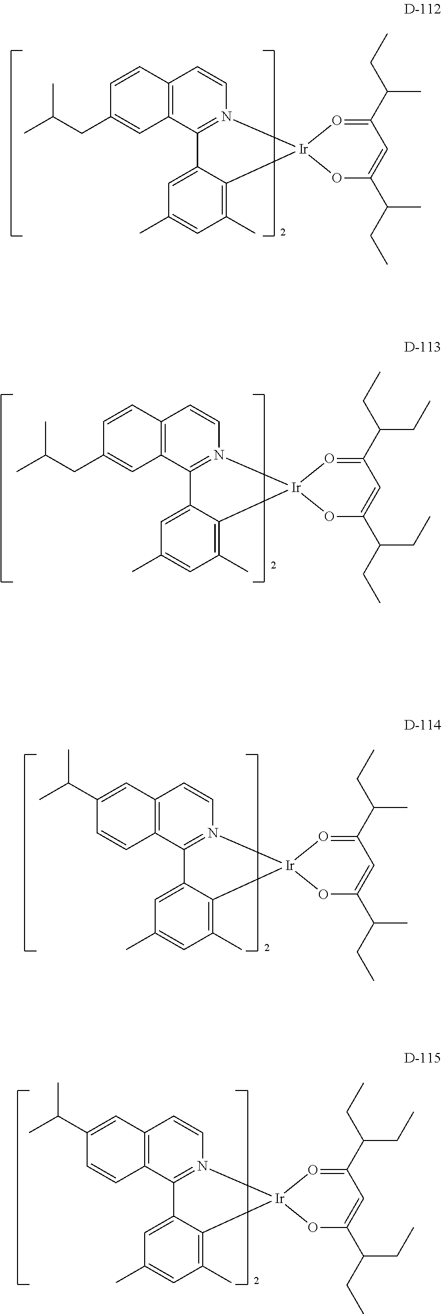

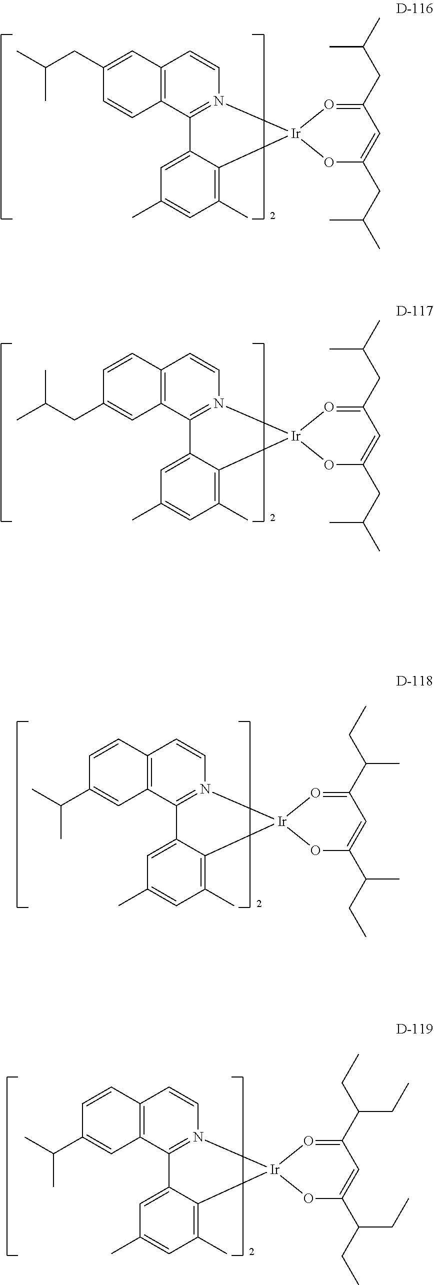

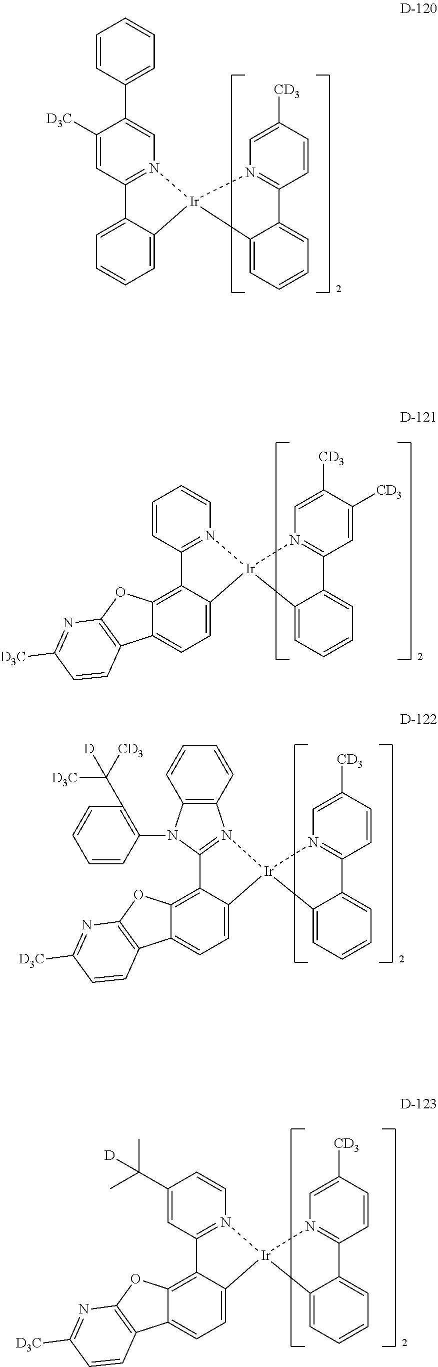

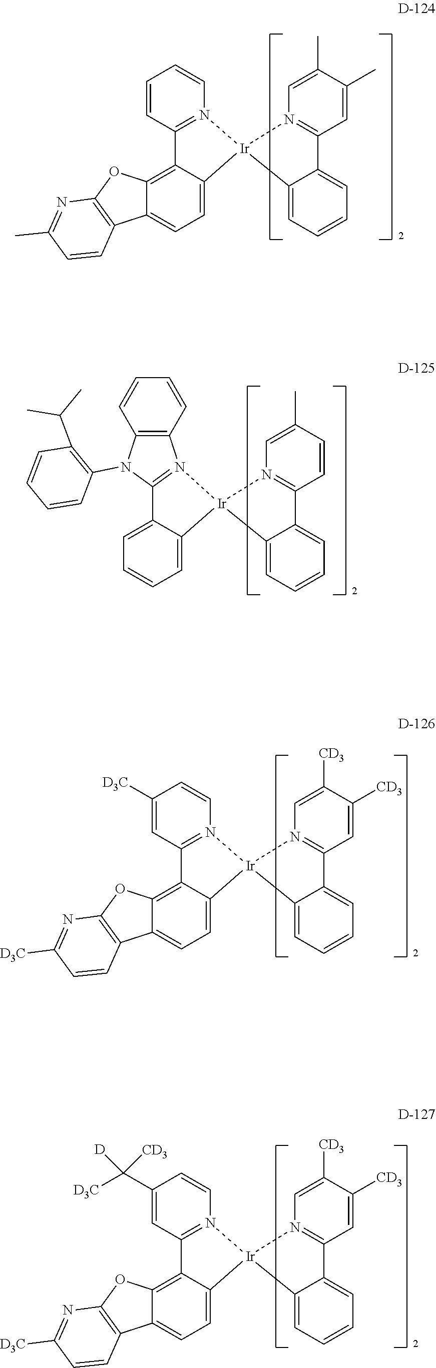

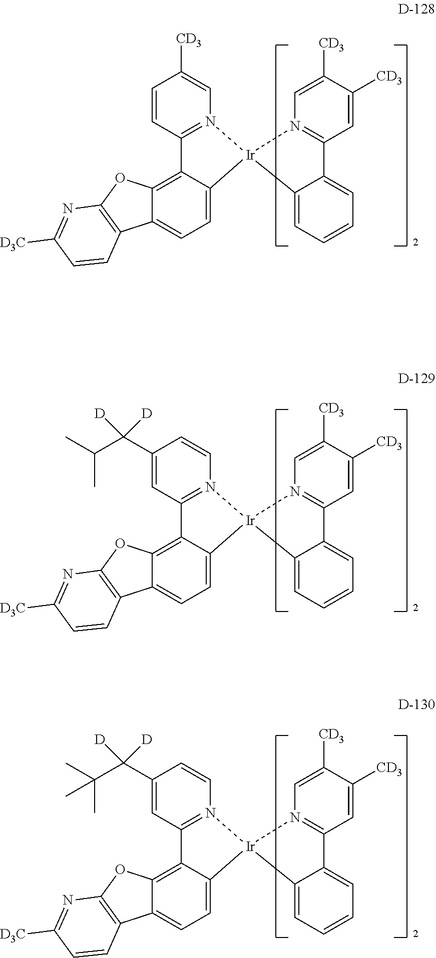

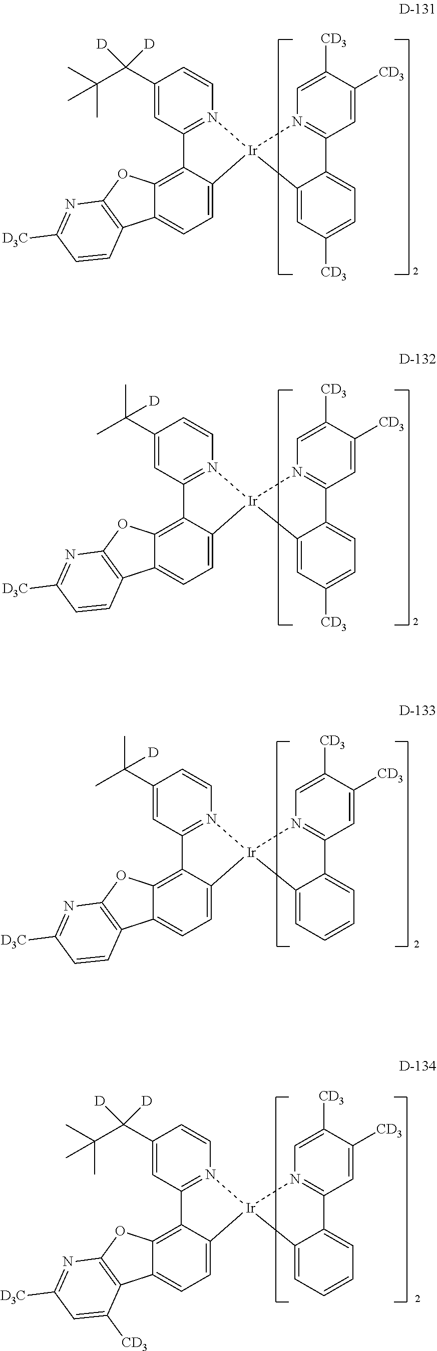

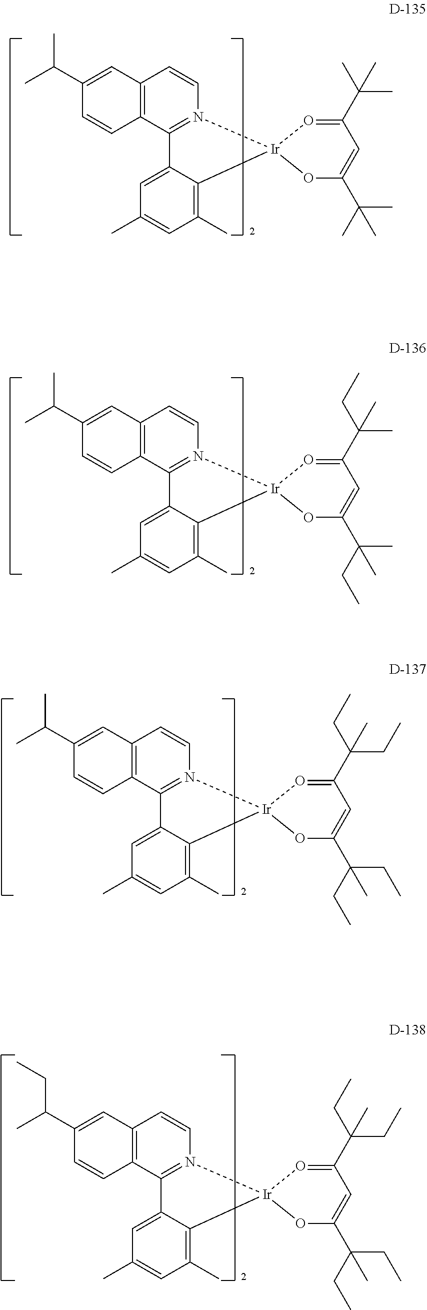

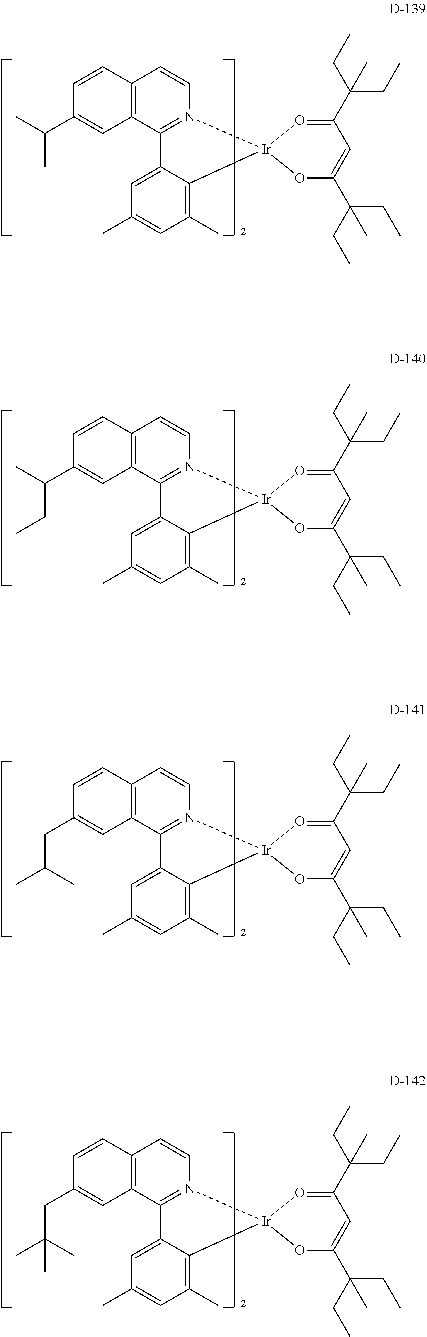

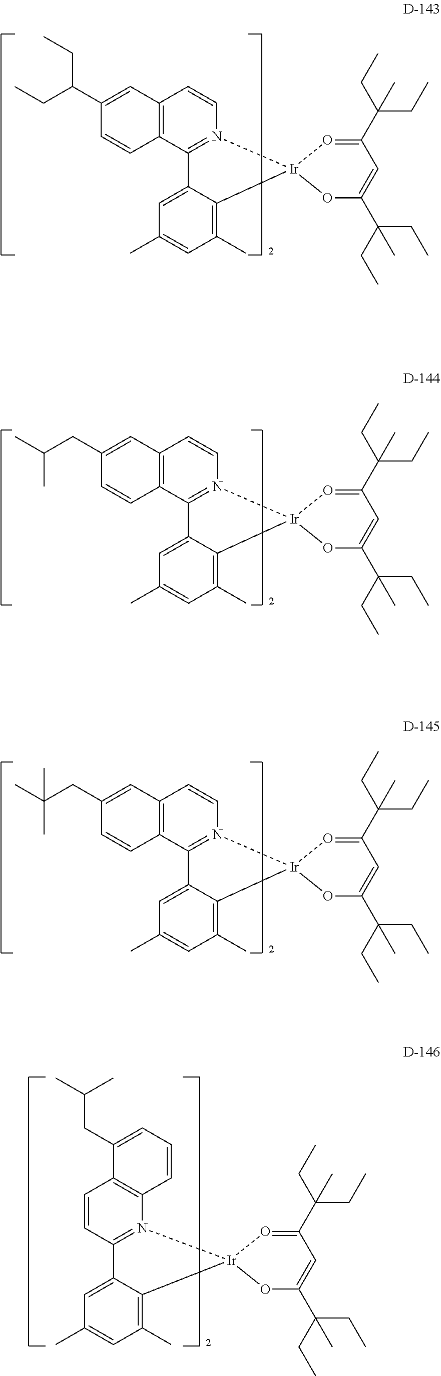

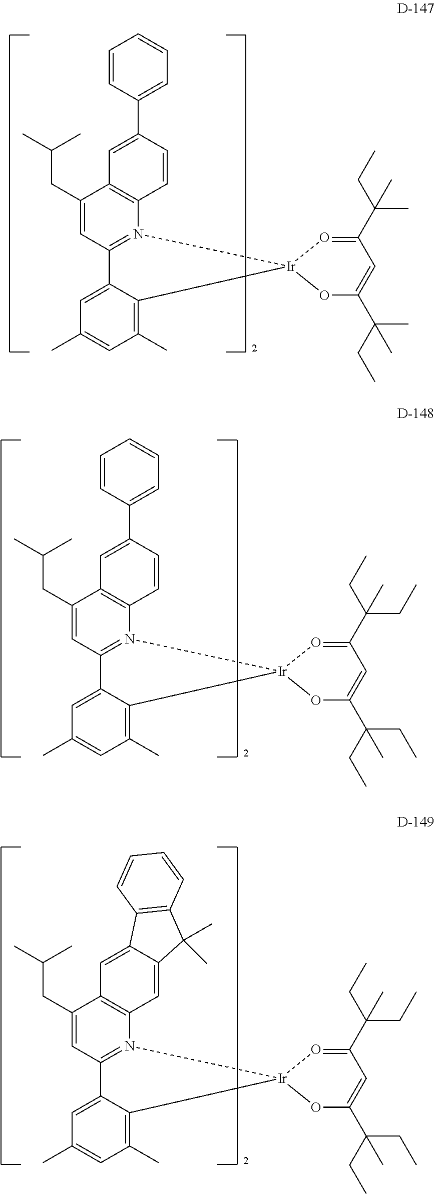

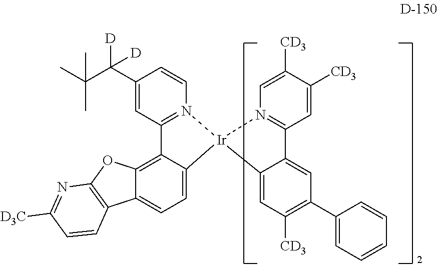

An organic electroluminescent device according to the present disclosure comprises an anode; a cathode; and at least one organic layer interposed between the anode and the cathode. The organic layer comprises a light-emitting layer and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as several layers.

Each of the anode and the cathode may be formed of a transparent conductive material or a transflective or reflective conductive material. Depending on the type of material forming the anode and the cathode, the organic electroluminescent device may be a top emission type, a bottom emission type, or a both-sides emission type. In addition, the hole injection layer may be further doped with a p-dopant, and the electron injection layer may be further doped with an n-dopant.

The organic layer may further comprise at least one compound selected from the group consisting of an arylamine-based compound and a styrylarylamine-based compound. In addition, the organic layer may further comprise at least one metals selected from the group consisting of metals of Group 1, metals of Group 2, transition metals of the 4^{th} period, transition metals of the 5^{th} period, lanthanides, and organic metals of d-transition elements, or at least one complex compound comprising such metal.

In addition, the organic electroluminescent device of the present disclosure may emit white light by further including at least one light-emitting layer including a blue, red, or green light-emitting compound known in the art, besides the compound of the present disclosure. In addition, if necessary, a yellow or orange light-emitting layer may be further included.

In the organic electroluminescent device of the present disclosure, at least one layer selected from a chalcogenide layer, a metal halide layer, and a metal oxide layer (hereinafter, “a surface layer”) may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. The surface layer may provide operation stability for the organic electroluminescent device. Preferably, the chalcogenide includes SiO_{X} (1≤X≤2), AlO_{X} (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer, a hole transport layer, an electron blocking layer, or a combination thereof may be used between an anode and a light-emitting layer. The hole injection layer may be multilayers in order to lower the hole injection barrier (or hole injection voltage) from the anode to the hole transport layer or the electron blocking layer, wherein two compounds may be simultaneously used in each of the multilayers. The hole transport layer or the electron blocking layer may also be multilayers.

An electron buffer layer, a hole blocking layer, an electron transport layer, an electron injection layer, or a combination thereof may be used between a light-emitting layer and a cathode. The electron buffer layer may be multilayers in order to control electron injection and improve interfacial properties between the light-emitting layer and the electron injection layer, wherein two compounds may be simultaneously used in each of the multilayers. The hole blocking layer or the electron transport layer may also be multilayers, wherein a plurality of compounds may be used in each of the multilayers.

The light-emitting auxiliary layer is a layer placed between an anode and a light-emitting layer, or between a cathode and a light-emitting layer. When the light-emitting auxiliary layer is placed between the anode and the light-emitting layer, it may be used to facilitate injection and/or transport of holes or to prevent the overflow of electrons. When the light-emitting auxiliary layer is placed between the cathode and the light-emitting layer, it may be used to facilitate injection and/or transport of electrons or to prevent the overflow of holes. In addition, the hole auxiliary layer may be placed between a hole transport layer (or a hole injection layer) and a light-emitting layer to exhibit an effect of facilitating or blocking transport rate (or injection rate) of holes, thereby enabling the charge balance to be controlled. The electron blocking layer may be placed between the hole transport layer (or hole injection layer) and the light-emitting layer, and may block overflow of electrons from the light-emitting layer and confine the excitons in the light-emitting layer to prevent light leakage. When the organic electroluminescent device comprises two or more hole transport layers, the hole transport layer, which is further comprised, may be used as a hole auxiliary layer or an electron blocking layer. The light-emitting auxiliary layer, the hole auxiliary layer, or the electron blocking layer has an effect of improving the efficiency and/or lifespan of the organic electroluminescent device.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to prepare an organic electroluminescent device having two or more light-emitting layers, which emits white light.

The organic electroluminescent materials according to one embodiment of the present disclosure may be used as light-emitting materials for a white organic light-emitting device. The white organic light-emitting device has been suggested in various structures such as a side-by-side structure or a stacking structure depending on the arrangement of R (Red), G (Green) or YG (Yellowish Green), and B (Blue) light-emitting parts, or a color conversion material (CCM) method, etc. In addition, the organic electroluminescent materials according to the present disclosure may also be used in the organic electroluminescent device comprising a quantum dot (QD).

Each layer of the organic electroluminescent device of the present disclosure may be formed by any one method of dry film-forming methods such as vacuum evaporation, sputtering, plasma, ion plating, etc., or wet film-forming methods such as ink jet printing, nozzle printing, slot coating, spin coating, dip coating, flow coating, etc. The first and the second host compounds according to the present disclosure may be film-formed by a co-evaporation process or a mixture-evaporation process.

When using a wet film-forming method, a thin film can be formed by dissolving or diffusing materials forming each layer into any suitable solvent such as ethanol, chloroform, tetrahydrofuran, dioxane, etc. The solvent can be any one where the material forming each layer is soluble or dispersible and where there are no problems in film formation capability.

In addition, it is possible to produce a display system, e.g., a display system for smartphones, tablets, notebooks, PCs, TVs, or cars, or a lighting system, e.g., an outdoor or indoor lighting system by using an organic electroluminescent device of the present disclosure.

Hereinafter, the preparation method of the compound of the present disclosure and physical properties thereof, and the driving voltage and luminous efficiency of the organic electroluminescent device (OLED) comprising a plurality of host materials according to the present disclosure will be explained in detail with reference to the representative compounds of the present disclosure. However, the following examples only explain the properties of the compound according to the present disclosure and the OLED comprising the same, and the present disclosure is not limited to the following examples.

### Example 1: Preparation of Compound C-109

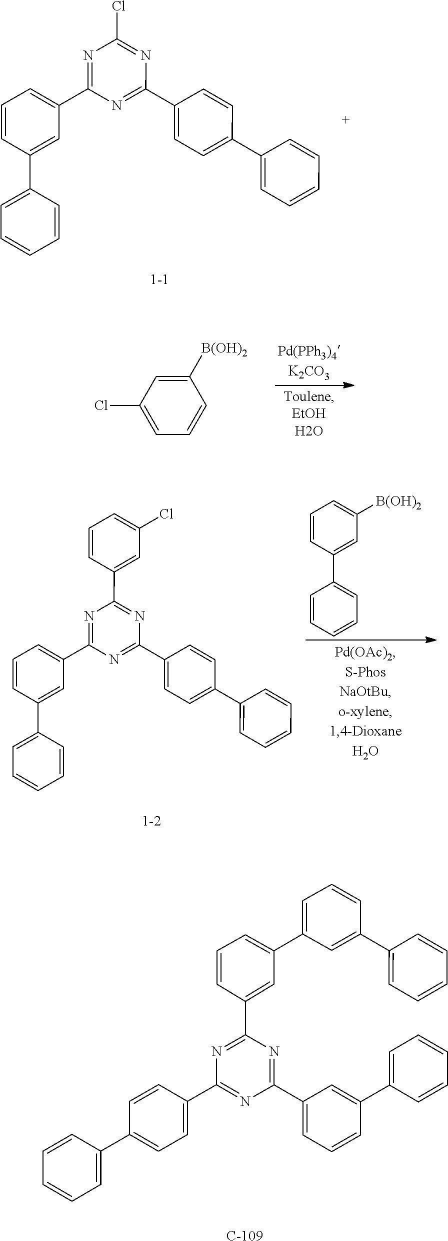 [SMILES: Clc1nc(-c2ccc(-c3ccccc3)cc2)nc(-c2cccc(-c3ccccc3)c2)n1 ; OB(O)c1cccc(Cl)c1 ; Clc1cccc(-c2nc(-c3ccc(-c4ccccc4)cc3)nc(-c3cccc(-c4ccccc4)c3)n2)c1 ; OB(O)c1cccc(-c2ccccc2)c1 ; c1ccc(-c2ccc(-c3nc(-c4cccc(-c5ccccc5)c4)nc(-c4cccc(-c5cccc(-c6ccccc6)c5)c4)n3)cc2)cc1 ; "C-109"]

### Synthesis of Compound 1-2

(3-chlorophenyl)boronic acid (2.6 g, 16.67 mmol), compound 1-1 (7 g, 16.67 mmol), tetrakis(triphenylphosphine)palladium (5.8 mg, 0.5 mmol), and potassium carbonate (5.8 g, 41.7 mmol) were dissolved in 83 mL of toluene, 21 mL of ethanol, and 21 mL of water in a reaction vessel and stirred under reflux for 4 hours. After cooling to room temperature, water was added to the reactant in which a solid was formed and stirred for 30 minutes, followed by filtration. The filtrate was separated by column chromatography to obtain compound 1-2 (5.3 g, yield: 64%).

### Synthesis of Compound C-109

Compound 1-2 (5.3 g, 10.7 mmol), [1,1′-biphenyl]-3-yl boronic acid (2.54 g, 12.82 mmol), palladium acetate (0.25 g, 1.07 mmol), S-Phos (0.88 g, 2.14 mmol), and sodium tert-butoxide (3.1 g, 32.0 mmol) were dissolved in 53 mL of o-xylene, 13 mL of 1,4-dioxane, and 13 mL of water in a reaction vessel and stirred under reflux for 4 hours. After cooling to room temperature, water was added to the reactant in which a solid was formed and stirred for 30 minutes, followed by filtration. The filtrate was separated by column chromatography to obtain compound C-109 (2.9 g, yield: 44%).

| Compound | MW | M.P. |
|---|---|---|
| C-109 | 612.8 | 199 |

### Example 2: Preparation of Compound C-198

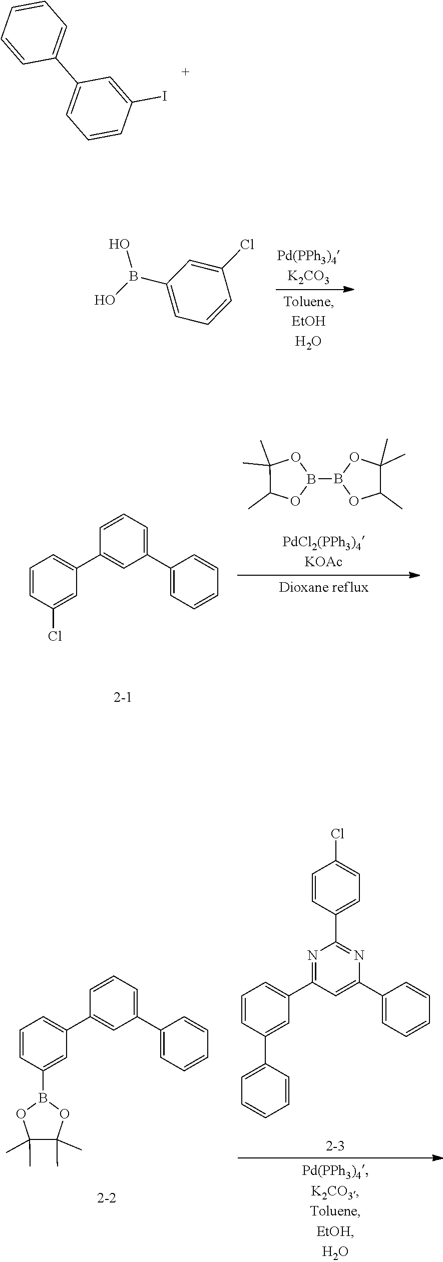

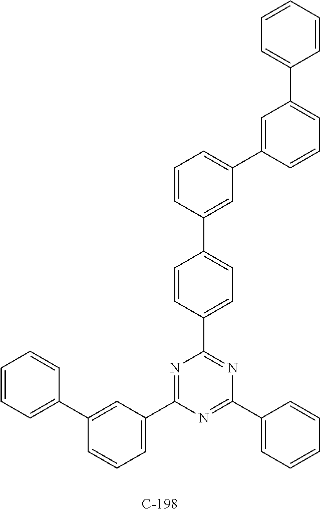 [SMILES: c1ccc(-c2cccc(-c3cccc(-c4ccc(-c5nc(-c6ccccc6)nc(-c6cccc(-c7ccccc7)c6)n5)cc4)c3)c2)cc1]

### Synthesis of Compound 2-1

3-chloro-1,1′-biphenyl (18 g, 63.9 mmol), (3-chlorophenyl)boronic acid (10 g, 63.9 mmol), tetrakis(triphenylphosphine)palladium (3.7 mg, 3.2 mmol), and potassium carbonate (22 g, 191 mmol) were dissolved in 320 mL of toluene, 160 mL of ethanol, and 160 mL of water in a reaction vessel and stirred under reflux for 4 hours. After cooling to room temperature, water was added to the reactant in which a solid was formed and stirred for 30 minutes, followed by filtration. The filtrate was separated by column chromatography to obtain compound 2-1 (15 g, yield: 89%).

### Synthesis of Compound 2-2

Compound 2-1 (6 g, 32.7 mmol), bis(pinacolato)diboron (11.5 g, 45.4 mmol), tris(dibenzylideneacetone)dipalladium(0) (2.1 g, 2.27 mmol), S-Phos (2.7 g, 6.54 mmol), and potassium acetate (6.7 g, 68.1 mmol) were dissolved in 110 mL of 1,4-dioxane in a reaction vessel and stirred under reflux for 3 hours. After the reaction was completed, the mixture was washed with distilled water and the organic layer was extracted with ethyl acetate. The organic layer was dried with magnesium sulfate and the solvent was removed using a rotary evaporator, followed by purification by column chromatography to obtain compound 2-2 (10 g, yield: 87%).

### Synthesis of Compound C-198

Compound 2-2 (8 g, 22.4 mmol), compound 2-3 (7.8 g, 18.7 mmol), palladium acetate (420 mg, 1.8 mmol), S-Phos (1.5 g, 3.7 mmol), and cesium carbonate (18 g, 56.1 mmol) were dissolved in 93 mL of toluene, 23 mL of ethanol, and 23 mL of water in a reaction vessel and stirred under reflux for 4 hours. After cooling to room temperature, water was added to the reactant in which a solid was formed and stirred for 30 minutes, followed by filtration. The filtrate was separated by column chromatography to obtain compound C-198 (3.3 g, yield: 30%).

| Compound | MW | M.P. |
|---|---|---|
| C-198 | 613.2 | 185.4 |

### Example 3: Preparation of Compound C-28

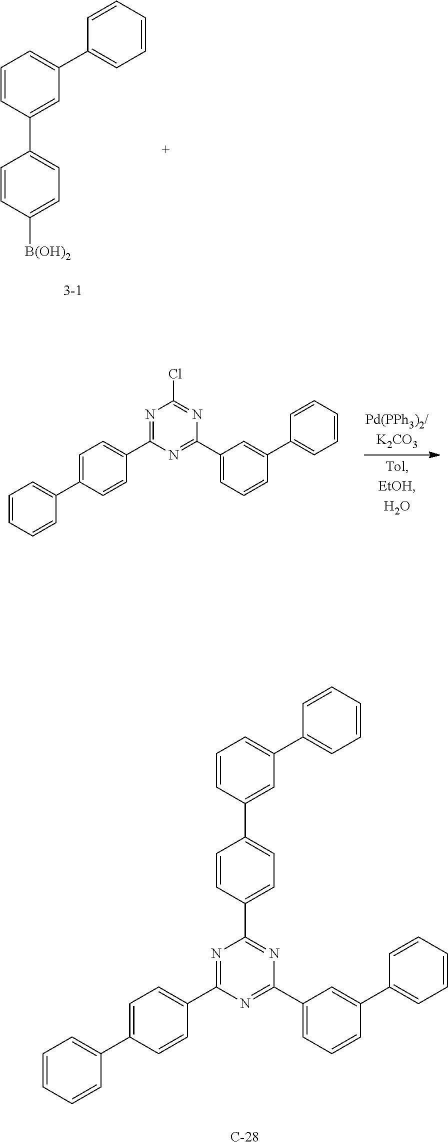

Compound 3-1 (6.0 g, 21.89 mmol), 2-([1,1′-biphenyl]-3-yl)-4-([1,1′-biphenyl]-4-yl)-6-chloro-1,3,5-triazine (7.6 g, 18.24 mmol), tetrakis(triphenylphosphine)palladium (0.7 g, 0.55 mmol), and potassium carbonate (6.3 g, 45.60 mmol) were dissolved in 91 mL of toluene, 23 mL of ethanol, and 23 mL of distilled water in a flask, and stirred under reflux for 4 hours. After the reaction was completed, the organic layer was extracted with ethyl acetate and separated by column chromatography to obtain compound C-28 (3.3 g, yield: 30%).

| Compound | MW | M.P. |
|---|---|---|
| C-28 | 613.75 | 220° C. |

### Example 4: Preparation of Compound C-266

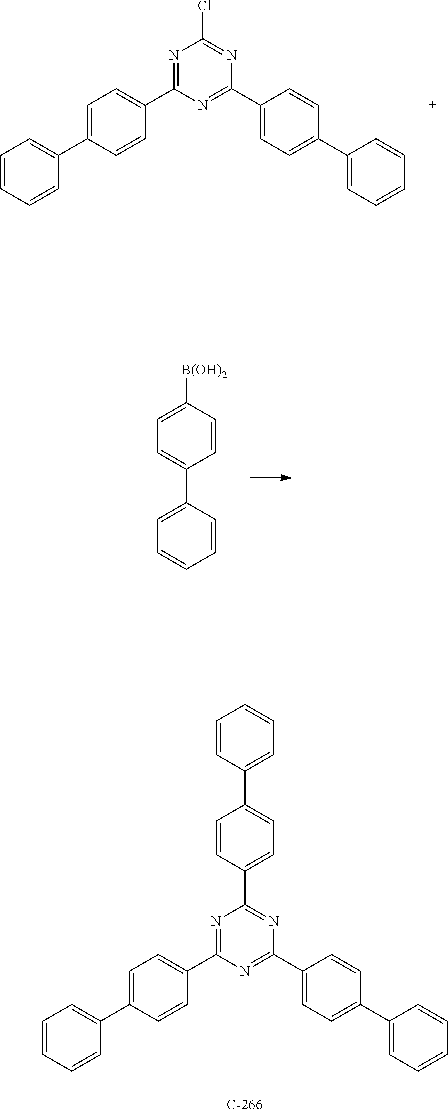 [SMILES: Clc1nc(-c2ccc(-c3ccccc3)cc2)nc(-c2ccc(-c3ccccc3)cc2)n1 ; OB(O)c1ccc(-c2ccccc2)cc1 ; c1ccc(-c2ccc(-c3nc(-c4ccc(-c5ccccc5)cc4)nc(-c4ccc(-c5ccccc5)cc4)n3)cc2)cc1 ; "C-266"]

2,4-di([1,1′-biphenyl]-4-yl)-6-chloro-1,3,5-triazine (5 g, 11.91 mmol), [1,1′-biphenyl]-4-yl boronic acid (2.6 g, 13.09 mmol), tetrakis(triphenylphosphine)palladium (0.688 g, 0.595 mmol), and sodium carbonate (4.1 g, 29.77 mmol) were dissolved in 100 mL of toluene, 50 mL of ethanol, and 50 mL of distilled water in a flask, and stirred under reflux for 4 hours. After the reaction was completed, the organic layer was extracted with ethyl acetate and separated by column chromatography to obtain compound C-266 (5.5 g, yield: 86%).

| Compound | MW | M.P. |
|---|---|---|
| C-266 | 537.67 | 265.9° C. |

### Example 5: Preparation of Compound C-199

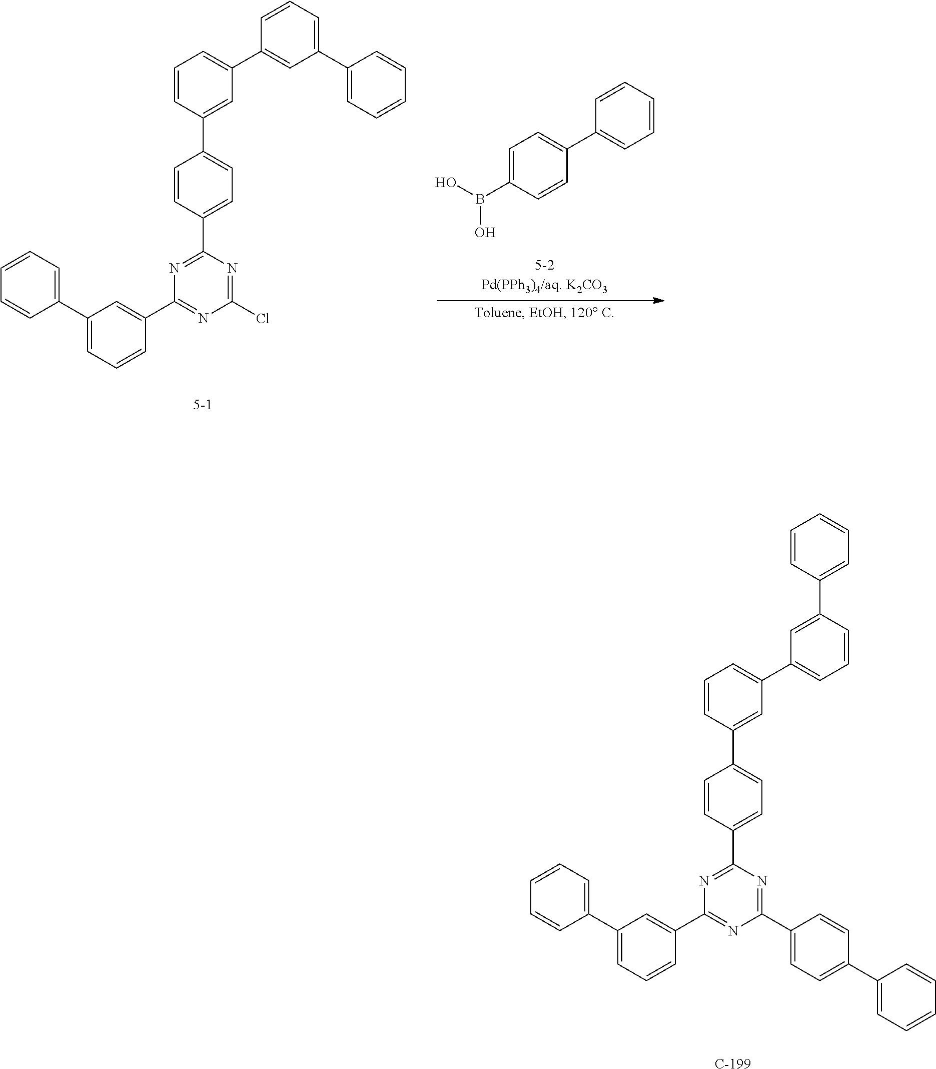 [SMILES: Clc1nc(-c2ccc(-c3cccc(-c4cccc(-c5ccccc5)c4)c3)cc2)nc(-c2cccc(-c3ccccc3)c2)n1 ; OB(O)c1ccc(-c2ccccc2)cc1 ; "5-1" ; c1ccc(-c2ccc(-c3nc(-c4ccc(-c5cccc(-c6cccc(-c7ccccc7)c6)c5)cc4)nc(-c4cccc(-c5ccccc5)c4)n3)cc2)cc1 ; "C-199"]

Compound 5-1 (3.4 g, 5.95 mmol), compound 5-2 (1.2 g, 5.95 mmol), tetrakis(triphenylphosphine)palladium (343 mg, 0.29 mmol), potassium carbonate (2.5 g, 17.85 mmol), 30 mL of toluene, 15 mL of ethanol, and 15 mL of distilled water were added to a reaction vessel and stirred at 120° C. for 3 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-199 (1.9 g, yield: 46%).

| Compound | MW | M.P. |
|---|---|---|
| C-199 | 689.86 | 232° C. |

### Example 6: Preparation of Compound C-370

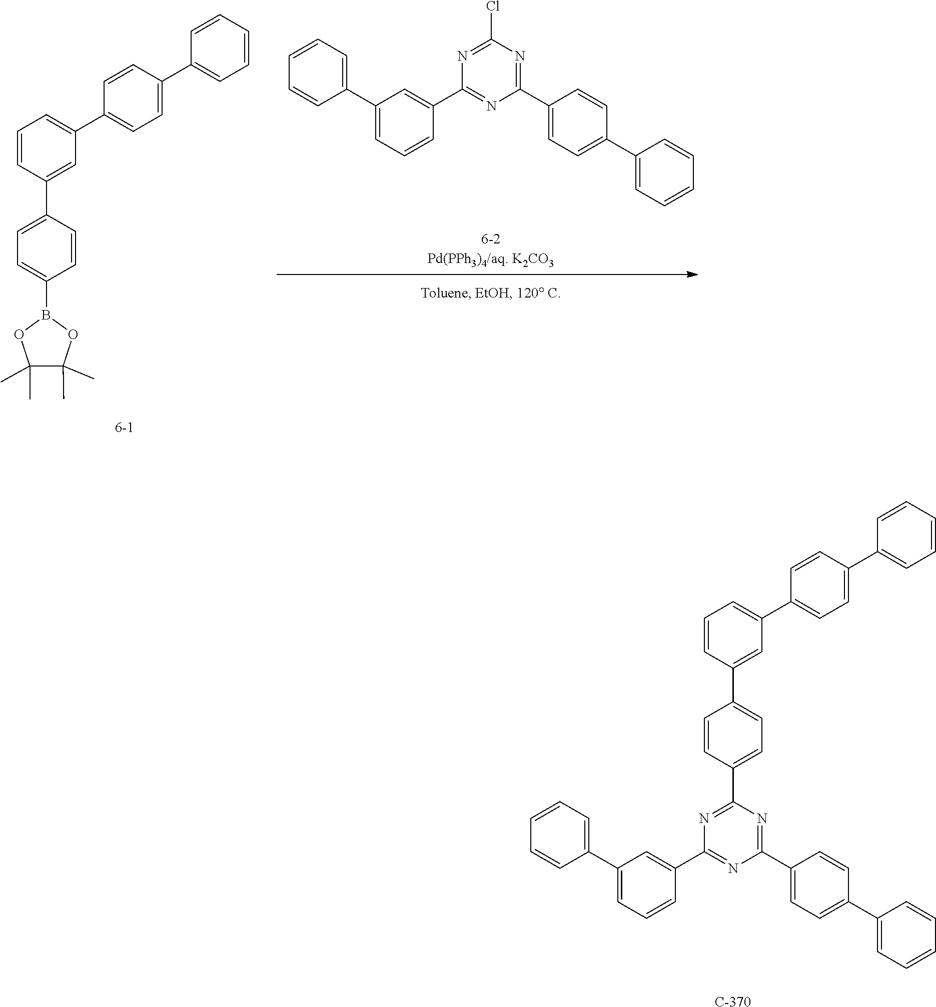

Compound 6-1 (5 g, 11.56 mmol), compound 6-2 (5.3 g, 12.72 mmol), tetrakis(triphenylphosphine)palladium (400 mg, 0.35 mmol), potassium carbonate (4 g, 28.91 mmol), 57 mL of toluene, 14 mL of ethanol, and 14 mL of distilled water were added to a reaction vessel and stirred at 120° C. for 3 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-370 (4.9 g, yield: 61%).

| Compound | MW | M.P. |
|---|---|---|
| C-370 | 689.86 | 302.7° C. |

### Example 7: Preparation of Compound C-17

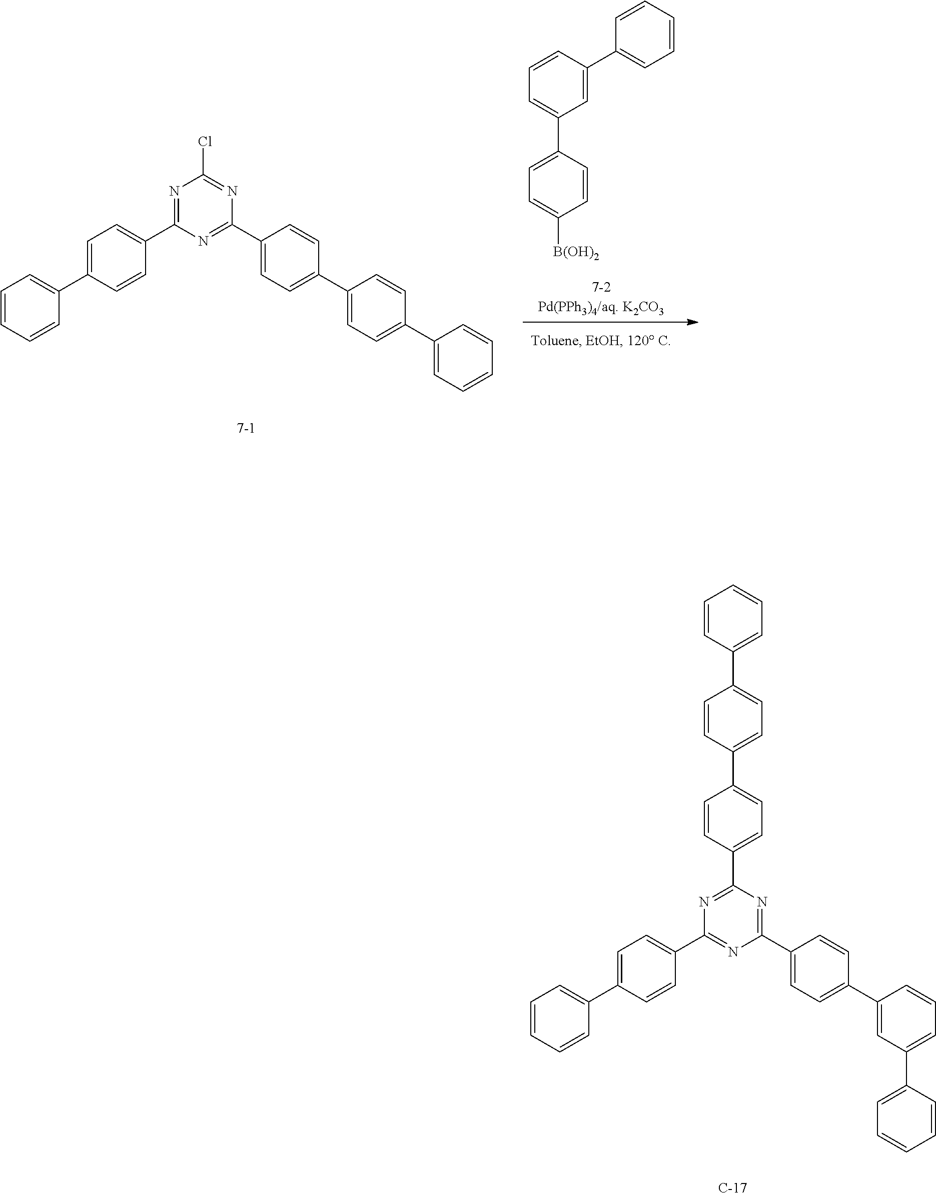

Compound 7-1 (5 g, 10.08 mmol), compound 7-2 (3.3 g, 12.10 mmol), tetrakis(triphenylphosphine)palladium (350 mg, 0.30 mmol), potassium carbonate (3.5 g, 25.20 mmol), 50 mL of toluene, 13 mL of ethanol, and 13 mL of distilled water were added to a reaction vessel and stirred at 120° C. for 3 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-17 (3.4 g, yield: 49%).

| Compound | MW | M.P. |
|---|---|---|
| C-17 | 689.86 | 243° C. |

### Example 8: Preparation of Compound C-371

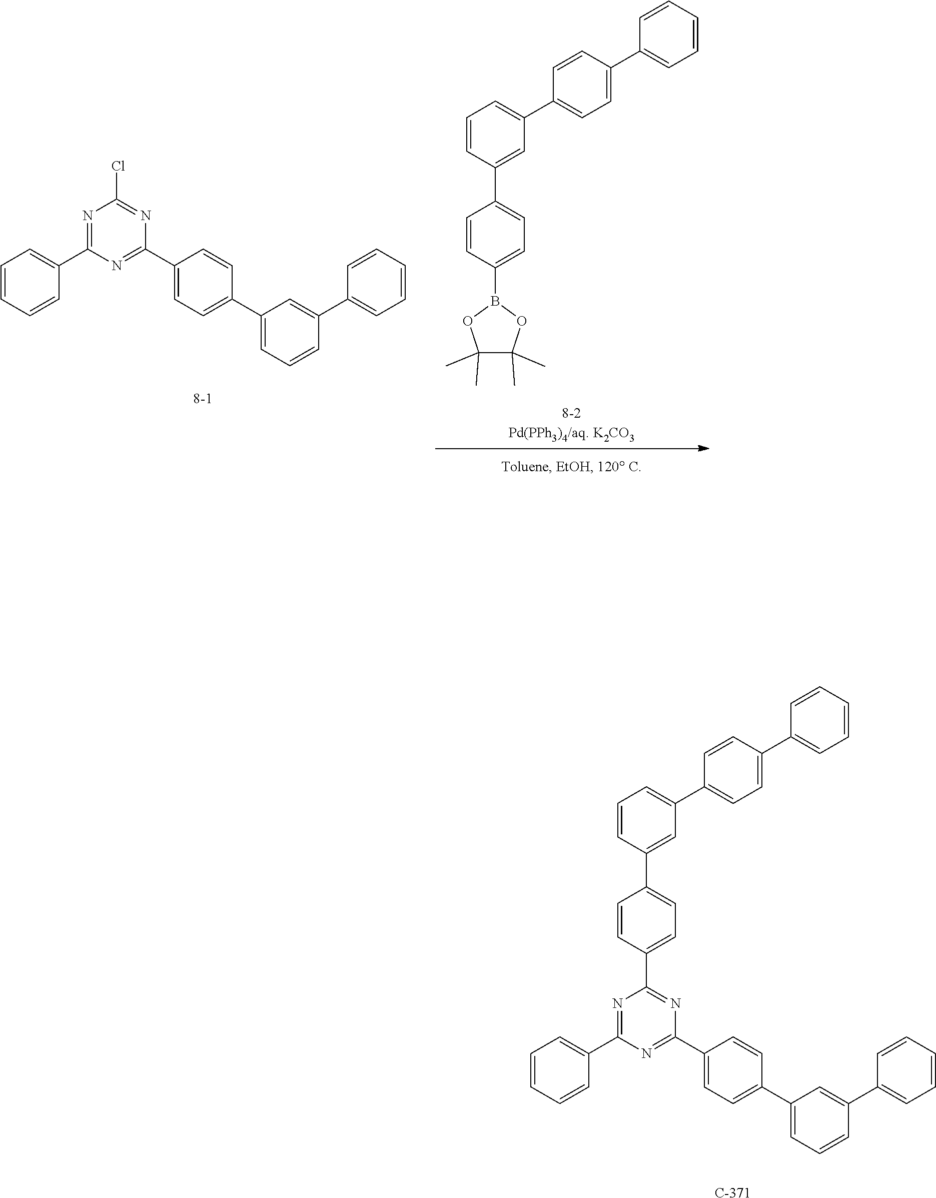 [SMILES: Clc1nc(-c2ccccc2)nc(-c2ccc(-c3cccc(-c4ccccc4)c3)cc2)n1 ; CC1(C)OB(c2ccc(-c3cccc(-c4ccc(-c5ccccc5)cc4)c3)cc2)OC1(C)C ; "8-1" ; c1ccc(-c2ccc(-c3cccc(-c4ccc(-c5nc(-c6ccccc6)nc(-c6ccc(-c7cccc(-c8ccccc8)c7)cc6)n5)cc4)c3)cc2)cc1 ; "C-371"]

Compound 8-1 (5 g, 11.91 mmol), compound 8-2 (5.2 g, 11.91 mmol), tetrakis(triphenylphosphine)palladium (410 mg, 0.30 mmol), potassium carbonate (4.1 g, 29.77 mmol), 60 mL of toluene, 15 mL of ethanol, and 15 mL of distilled water were added to a reaction vessel and stirred at 120° C. for 3 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-371 (5.5 g, yield: 67%).

| Compound | MW | M.P. |
|---|---|---|
| C-371 | 689.86 | 242° C. |

### Example 9: Preparation of Compound C-372

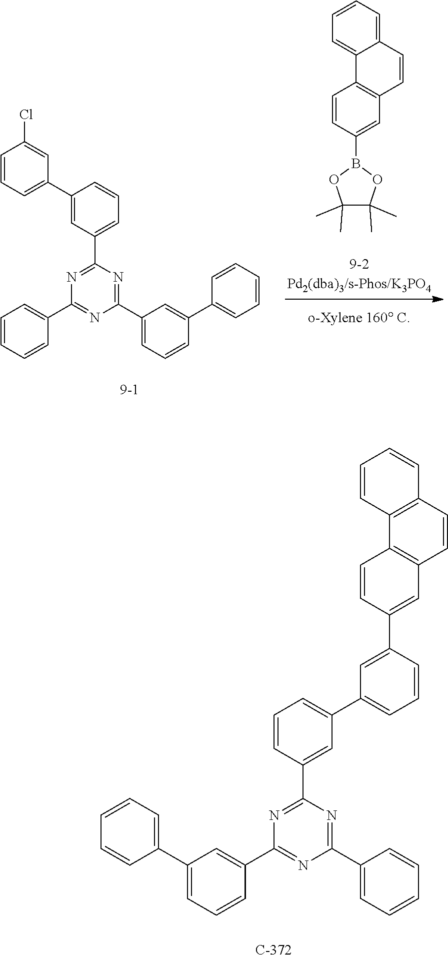 [SMILES: Clc1cccc(-c2cccc(-c3nc(-c4ccccc4)nc(-c4cccc(-c5ccccc5)c4)n3)c2)c1 ; CC1(C)OB(c2ccc3c(ccc4ccccc43)c2)OC1(C)C ; "9-1" ; c1ccc(-c2cccc(-c3nc(-c4ccccc4)nc(-c4cccc(-c5cccc(-c6ccc7c(ccc8ccccc87)c6)c5)c4)n3)c2)cc1 ; "C-372"]

Compound 9-1 (7.1 g, 14.30 mmol), compound 9-2 (6.5 g, 21.30 mmol), tris(dibenzylideneacetone)dipalladium (651 mg, 0.7 mmol), S-Phos (587 mg, 1.4 mmol), and potassium phosphate (9.1 g, 42.8 mmol) were dissolved in 100 mL of xylene in a reaction vessel and stirred at 160° C. for 24 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-372 (2.1 g, yield: 23%).

| Compound | MW | M.P. |
|---|---|---|
| C-372 | 637.79 | 215° C. |

### Example 10: Preparation of Compound C-373

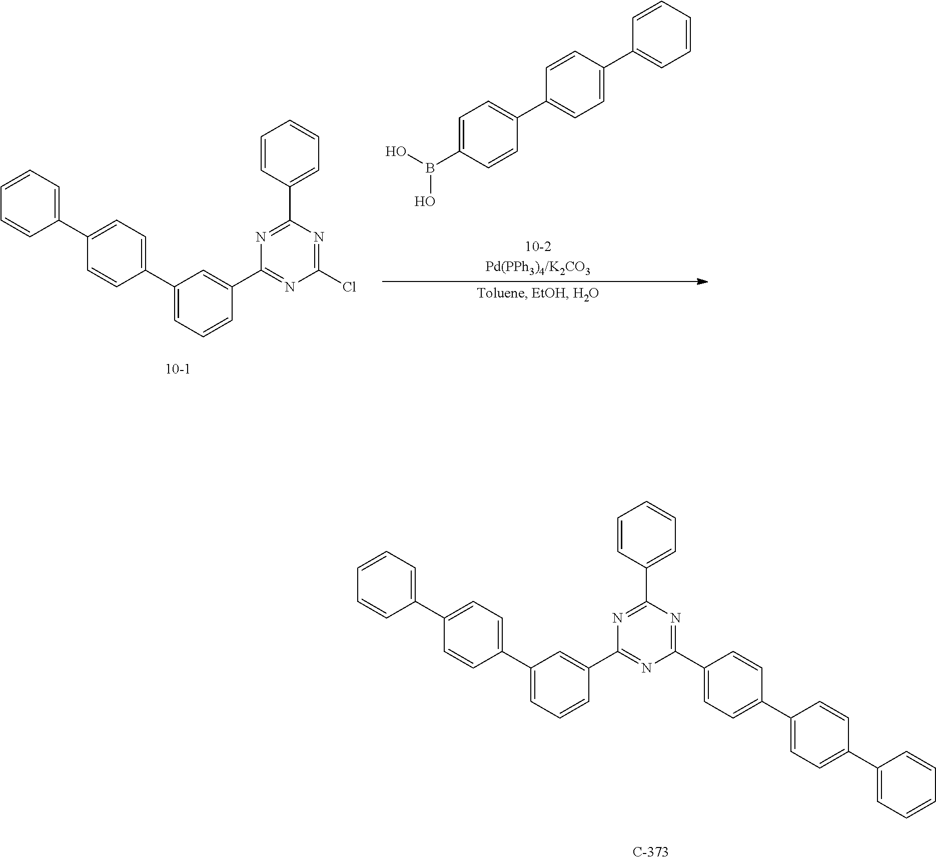 [SMILES: Clc1nc(-c2ccccc2)nc(-c2cccc(-c3ccc(-c4ccccc4)cc3)c2)n1 ; OB(O)c1ccc(-c2ccc(-c3ccccc3)cc2)cc1 ; c1ccc(-c2ccc(-c3ccc(-c4nc(-c5ccccc5)nc(-c5cccc(-c6ccc(-c7ccccc7)cc6)c5)n4)cc3)cc2)cc1]

Compound 10-1 (7.66 g, 18.2 mmol), compound 10-2 (5.00 g, 18.2 mmol), tetrakis(triphenylphosphine)palladium (632 mg, 0.547 mmol), potassium carbonate (7.56 g, 54.7 mmol), 500 mL of toluene, 100 mL of ethanol, and 100 mL of distilled water were added to a reaction vessel and stirred at 120° C. for 2 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-373 (6.7 g, yield: 60%).

| Compound | MW | M.P. |
|---|---|---|
| C-373 | 613.75 | 358° C. |

### Example 11: Preparation of Compound C-374

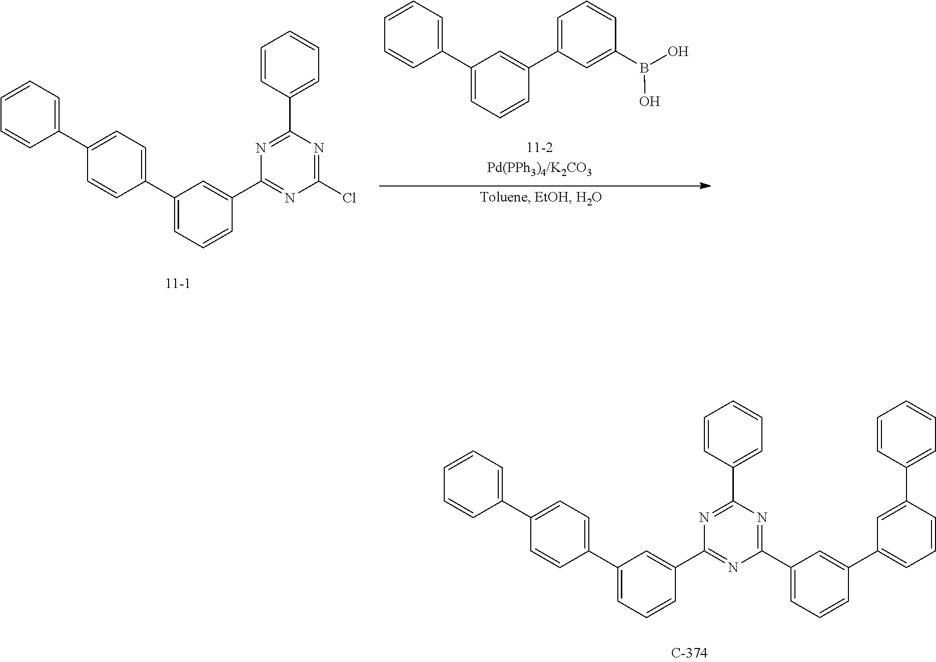 [SMILES: Clc1nc(-c2ccccc2)nc(-c2cccc(-c3ccc(-c4ccccc4)cc3)c2)n1 ; OB(O)c1cccc(-c2cccc(-c3ccccc3)c2)c1 ; c1ccc(-c2ccc(-c3cccc(-c4nc(-c5ccccc5)nc(-c5cccc(-c6cccc(-c7ccccc7)c6)c5)n4)c3)cc2)cc1]

Compound 11-1 (7.66 g, 18.2 mmol), compound 11-2 (5.00 g, 18.2 mmol), tetrakis(triphenylphosphine)palladium (632 mg, 0.547 mmol), potassium carbonate (7.56 g, 54.7 mmol), 500 mL of toluene, 100 mL of ethanol, and 100 mL of distilled water were added to a reaction vessel and stirred at 120° C. for 2 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-374 (6.7 g, yield: 14%).

| Compound | MW | M.P |
|---|---|---|
| C-374 | 613.75 | 232° C. |

### Example 12: Preparation of Compound C-375

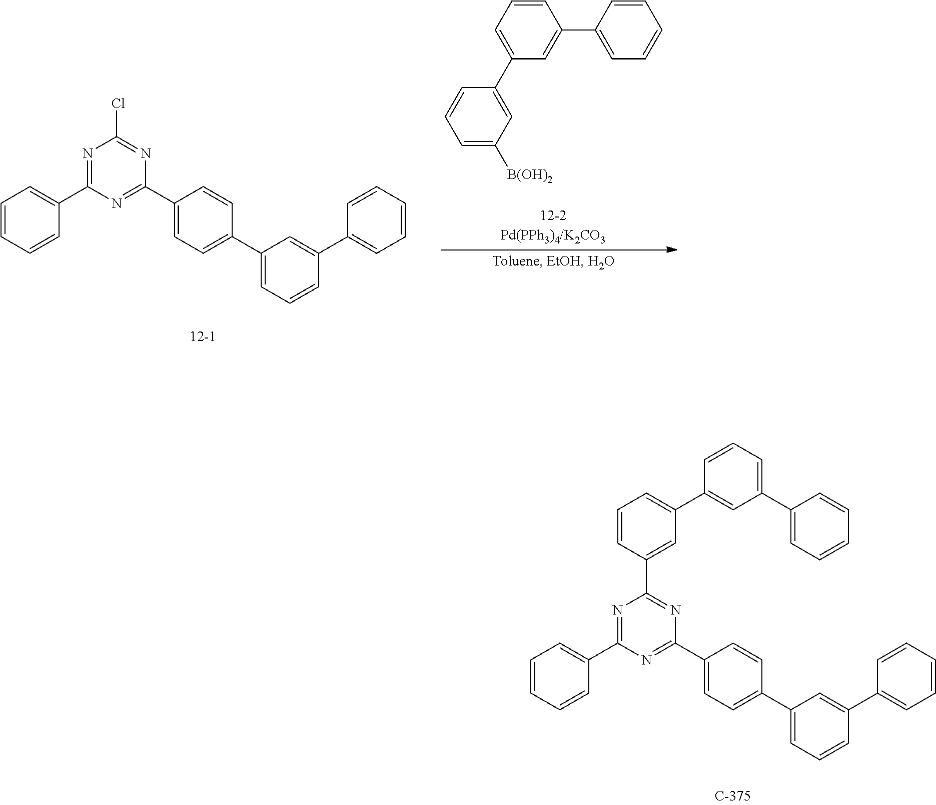 [SMILES: Clc1nc(-c2ccccc2)nc(-c2ccc(-c3cccc(-c4ccccc4)c3)cc2)n1 ; OB(O)c1cccc(-c2cccc(-c3ccccc3)c2)c1 ; c1ccc(-c2cccc(-c3ccc(-c4nc(-c5ccccc5)nc(-c5cccc(-c6cccc(-c7ccccc7)c6)c5)n4)cc3)c2)cc1]

Compound 12-1 (7.66 g, 18.2 mmol), compound 12-2 (5.00 g, 18.2 mmol), tetrakis(triphenylphosphine)palladium (632 mg, 0.547 mmol), potassium carbonate (7.56 g, 54.7 mmol), 500 mL of toluene, 100 mL of ethanol, and 100 mL of distilled water were added to a reaction vessel and stirred at 120° C. for 6 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-375 (3.7 g, yield: 33%).

| Compound | MW | M.P. |
|---|---|---|
| C-375 | 613.75 | 192.8° C. |

### Example 13: Preparation of Compound C-376

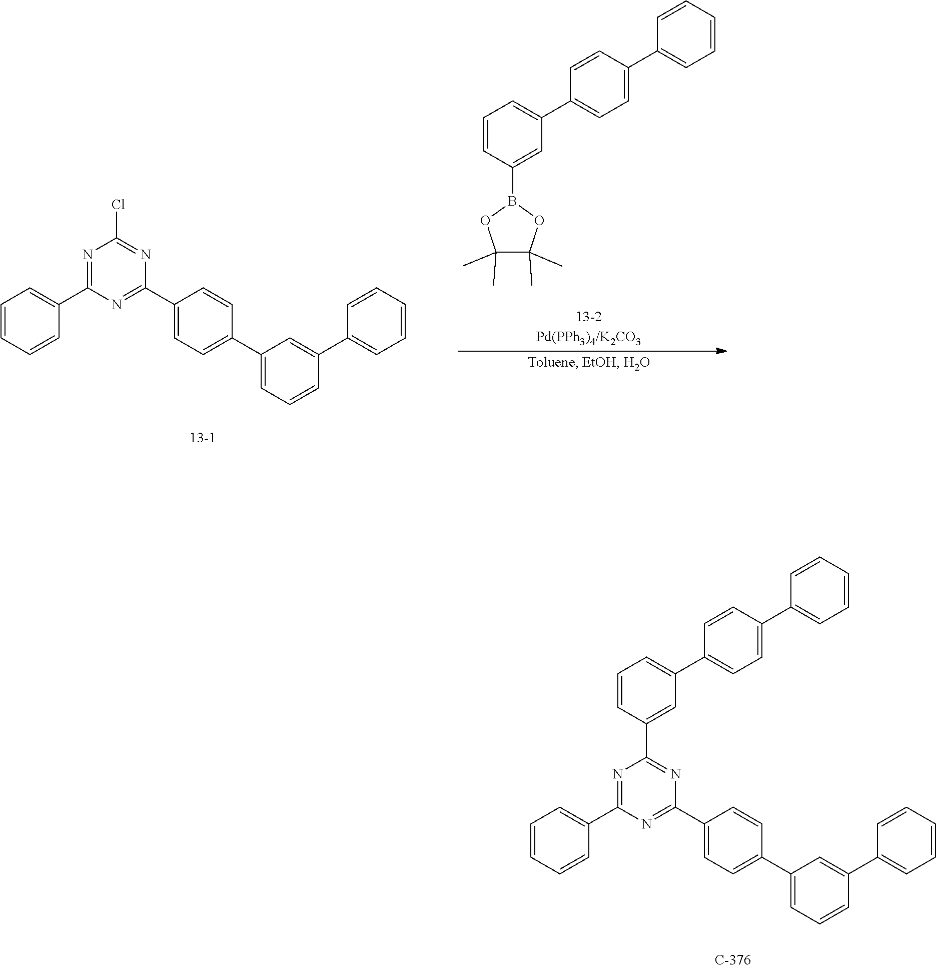 [SMILES: Clc1nc(-c2ccccc2)nc(-c2ccc(-c3cccc(-c4ccccc4)c3)cc2)n1 ; CC1(C)OB(c2cccc(-c3ccc(-c4ccccc4)cc3)c2)OC1(C)C ; c1ccc(-c2ccc(-c3cccc(-c4nc(-c5ccccc5)nc(-c5ccc(-c6cccc(-c7ccccc7)c6)cc5)n4)c3)cc2)cc1]

Compound 13-1 (7.66 g, 18.2 mmol), compound 13-2 (5.00 g, 18.2 mmol), tetrakis(triphenylphosphine)palladium (632 mg, 0.547 mmol), potassium carbonate (7.56 g, 54.7 mmol), 500 mL of toluene, 100 mL of ethanol, and 100 mL of distilled water were added to a reaction vessel and stirred at 120° C. for 6 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-376 (3.2 g, yield: 29%).

| Compound | MW | M.P. |
|---|---|---|
| C-376 | 613.75 | 245.9° C. |

### Example 14: Preparation of Compound C-377

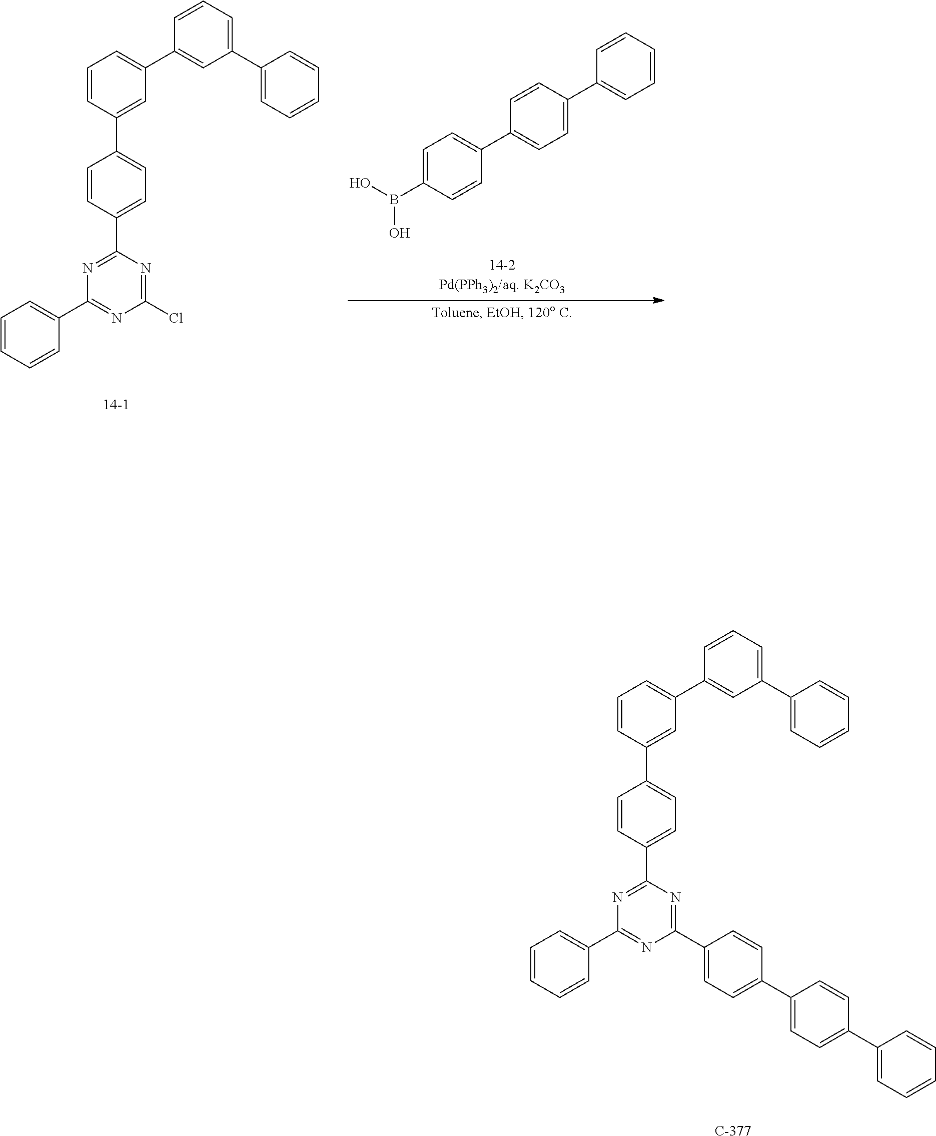 [SMILES: Clc1nc(-c2ccccc2)nc(-c2ccc(-c3cccc(-c4cccc(-c5ccccc5)c4)c3)cc2)n1 ; OB(O)c1ccc(-c2ccc(-c3ccccc3)cc2)cc1 ; "14-1" ; c1ccc(-c2ccc(-c3ccc(-c4nc(-c5ccccc5)nc(-c5ccc(-c6cccc(-c7cccc(-c8ccccc8)c7)c6)cc5)n4)cc3)cc2)cc1 ; "C-377"]

Compound 14-1 (3.4 g, 6.854 mmol), compound 14-2 (1.88 g, 6.854 mmol), tetrakis(triphenylphosphine)palladium (400 mg, 0.342 mmol), potassium carbonate (2.8 g, 20.56 mmol), 40 mL of toluene, 10 mL of ethanol, and 10 mL of distilled water were added to a reaction vessel and stirred at 120° C. for 2 hours. After the reaction was completed, the mixture was cooled to room temperature and the organic layer was extracted with ethyl acetate. The organic layer was dried with magnesium sulfate and the solvent was removed by a rotary evaporator, followed by purification by column chromatography to obtain compound C-377 (3.3 g, yield: 69%).

| Compound | MW | M.P |
|---|---|---|
| C-377 | 689.8 | 235° C. |

### Device Examples 1 to 4: Producing an OLED Comprising a Plurality of Host Materials According to the Present Disclosure

An OLED according to the present disclosure was produced. A transparent electrode indium tin oxide (ITO) thin film (10Ω/sq) on a glass substrate for an OLED (GEOMATEC CO., LTD., Japan) was subjected to an ultrasonic washing with acetone, and isopropyl alcohol, sequentially, and then was stored in isopropyl alcohol. The ITO substrate was mounted on a substrate holder of a vacuum vapor deposition apparatus. Compound HI-1 of Table 3 was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a hole injection layer with a thickness of 10 nm. Subsequently, compound HT-1 was deposited on the hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 30 nm on the first hole transport layer. After forming the hole injection layer and the hole transport layers, a light-emitting layer was deposited thereon as follows: A first host compound and a second host compound shown in Table 1 below were introduced into two cells of the vacuum vapor deposition apparatus as hosts and compound D-130 was introduced into another cell as a dopant. The two host materials were evaporated at a rate of 1:2 (a first host: a second host) and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Compound ETL-1 and compound EIL-1 as electron transport materials were evaporated in a weight ratio of 40:60 to deposit an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. Each compound for each material was purified by vacuum sublimation under 10⁻⁶ torr before use.

### Device Examples 5 to 13: Producing an OLED Comprising a Plurality of Host Materials According to the Present Disclosure

OLEDs were produced in the same manner as in Device Example 1, except that the first host compound shown in Table 1 below as a host of a light-emitting layer and compound D-150 as a dopant were used.

### Comparative Example 1: Producing an OLED Comprising a Conventional Compound

An OLED was produced in the same manner as in Device Example 1, except that the first host compound and the second host compound shown in Table 1 below were used as hosts of a light-emitting layer.

The driving voltage, luminous efficiency, and light-emitting color at a luminance of 1,000 nit, and the time taken for reduction of luminance from 100% to 80% (lifespan: T80) at a luminance of 60,000 nit of the OLEDs produced in Device Examples 1 to 13 and Comparative Example 1 are provided in Table 1 below.

TABLE 1

| | | | Driv- | Lumi- | | |
|---|---|---|---|---|---|---|
| | | | ing | nous | Light- | Life- |
| | | | Volt- | Effi- | Emitt- | span |
| | First Host | Second Host | age | ciency | ing | (T80, |
| | Compound | Compound | (V) | (cd/A) | Color | hr) |
| Device Exam- ple 1 | 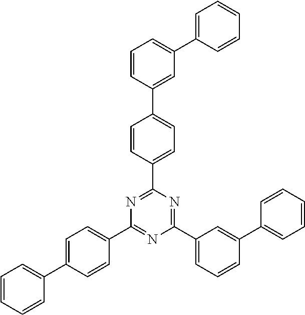 [SMILES: c1ccc(-c2ccc(-c3nc(-c4ccc(-c5cccc(-c6ccccc6)c5)cc4)nc(-c4cccc(-c5ccccc5)c4)n3)cc2)cc1] C-28 | 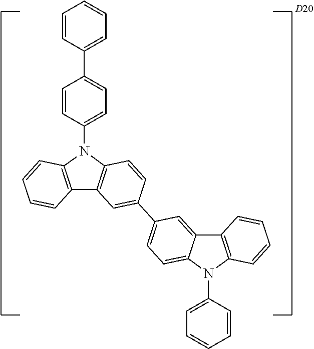 H2-2-D20 | 2.8 | 104.5 | Green | 155 |
| Device Exam- ple 2 | 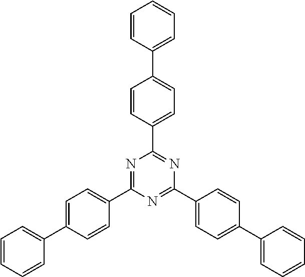 [SMILES: c1ccc(-c2ccc(-c3nc(-c4ccc(-c5ccccc5)cc4)nc(-c4ccc(-c5ccccc5)cc4)n3)cc2)cc1] C-266 | 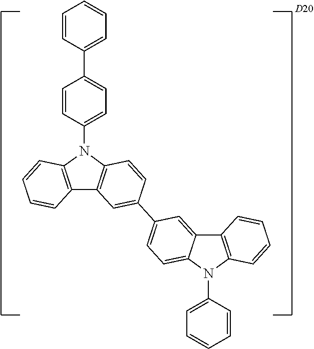 H2-2-D20 | 2.7 | 102.4 | Green | 80 |
| Device Exam- ple 3 | 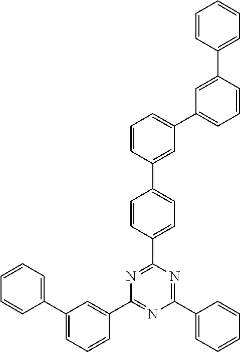 [SMILES: c1ccc(-c2cccc(-c3cccc(-c4ccc(-c5nc(-c6ccccc6)nc(-c6cccc(-c7ccccc7)c6)n5)cc4)c3)c2)cc1] C-198 | 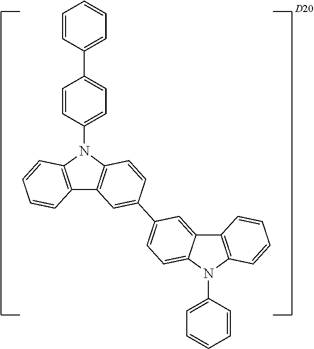 H2-2-D20 | 2.9 | 107.6 | Green | 199 |
| Device Exam- ple 4 | 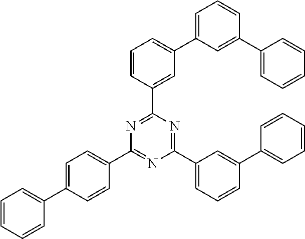 [SMILES: c1ccc(-c2ccc(-c3nc(-c4cccc(-c5ccccc5)c4)nc(-c4cccc(-c5cccc(-c6ccccc6)c5)c4)n3)cc2)cc1] C-109 | 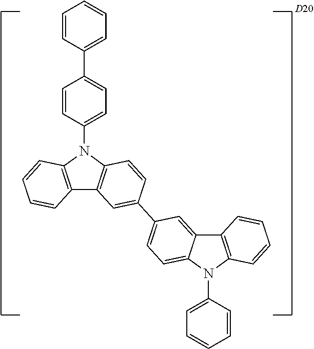 H2-2-D20 | 2.9 | 108.5 | Green | 221 |
| Device Exam- ple 5 | 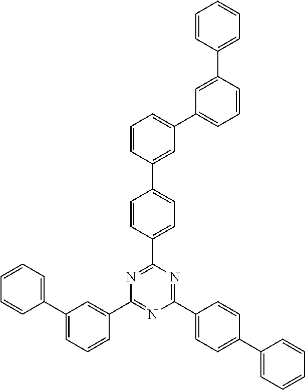 [SMILES: c1ccc(-c2ccc(-c3nc(-c4ccc(-c5cccc(-c6cccc(-c7ccccc7)c6)c5)cc4)nc(-c4cccc(-c5ccccc5)c4)n3)cc2)cc1] C-199 | 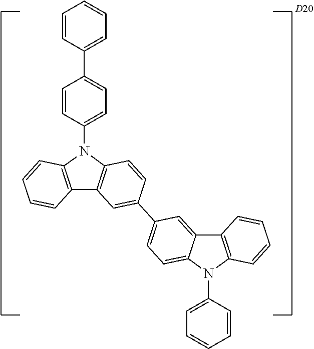 H2-2-D20 | 2.9 | 114.8 | Green | 130.4 |
| Device Exam- ple 6 | 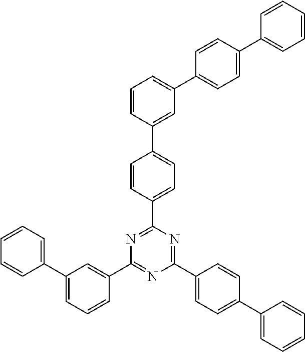 [SMILES: c1ccc(-c2ccc(-c3cccc(-c4ccc(-c5nc(-c6ccc(-c7ccccc7)cc6)nc(-c6cccc(-c7ccccc7)c6)n5)cc4)c3)cc2)cc1] C-370 | 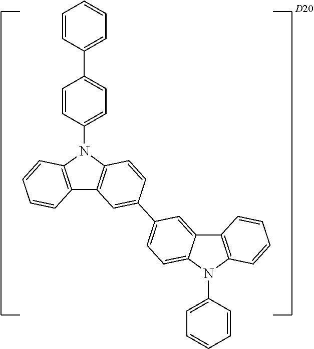 H2-2-D20 | 2.9 | 114.5 | Green | 136.3 |
| Device Exam- ple 7 | 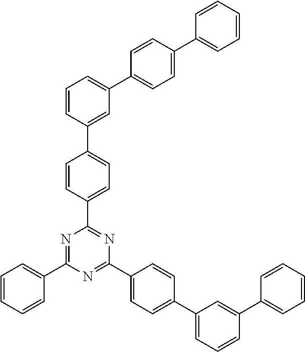 [SMILES: c1ccc(-c2ccc(-c3cccc(-c4ccc(-c5nc(-c6ccccc6)nc(-c6ccc(-c7cccc(-c8ccccc8)c7)cc6)n5)cc4)c3)cc2)cc1] C-371 | 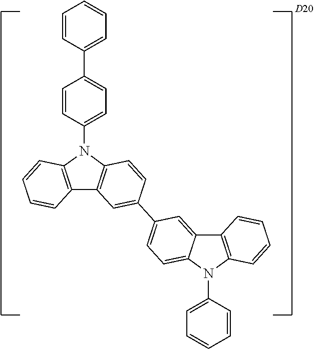 H2-2-D20 | 2.9 | 115.3 | Green | 130.4 |
| Device Exam- ple 8 | 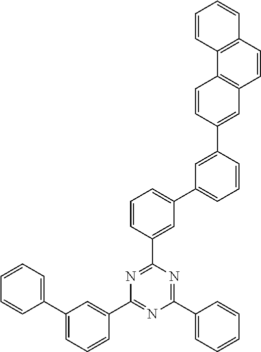 [SMILES: c1ccc(-c2cccc(-c3nc(-c4ccccc4)nc(-c4cccc(-c5cccc(-c6ccc7c(ccc8ccccc87)c6)c5)c4)n3)c2)cc1] C-372 | 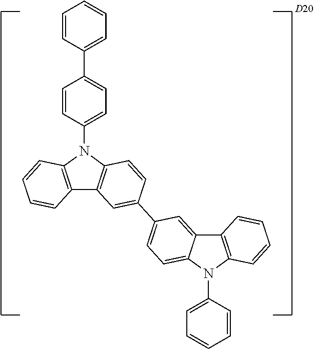 H2-2-D20 | 3.2 | 112.5 | Green | 120.9 |
| Device Exam- ple 9 | 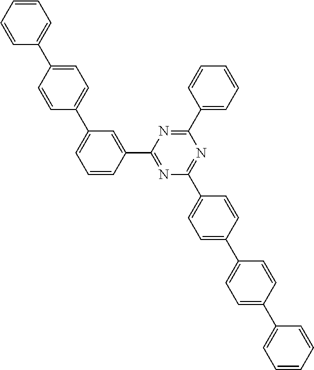 [SMILES: c1ccc(-c2ccc(-c3ccc(-c4nc(-c5ccccc5)nc(-c5cccc(-c6ccc(-c7ccccc7)cc6)c5)n4)cc3)cc2)cc1] C-373 | 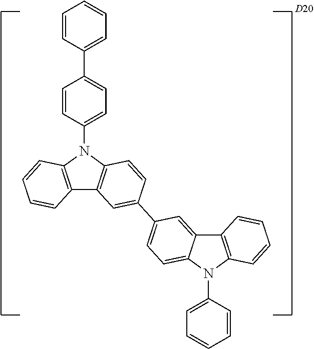 H2-2-D20 | 2.9 | 113.1 | Green | 81.5 |
| Device Exam- ple 10 | 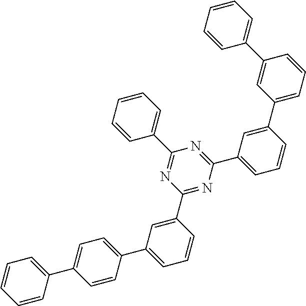 [SMILES: c1ccc(-c2ccc(-c3cccc(-c4nc(-c5ccccc5)nc(-c5cccc(-c6cccc(-c7ccccc7)c6)c5)n4)c3)cc2)cc1] C-374 | 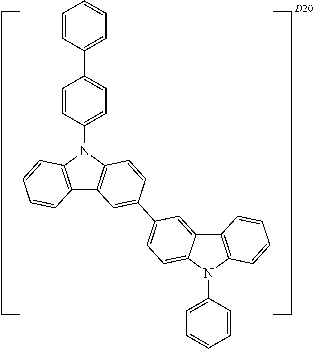 H2-2-D20 | 3.2 | 115.0 | Green | 123.0 |
| Device Exam- ple 11 | 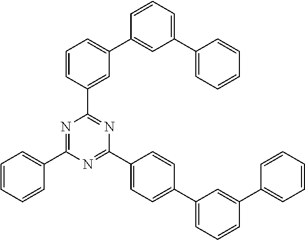 [SMILES: c1ccc(-c2cccc(-c3ccc(-c4nc(-c5ccccc5)nc(-c5cccc(-c6cccc(-c7ccccc7)c6)c5)n4)cc3)c2)cc1] C-375 | 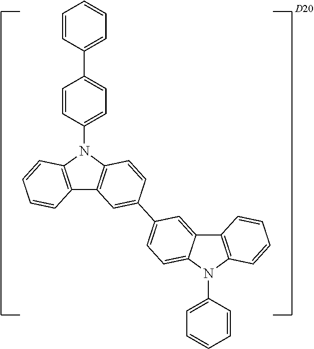 H2-2-D20 | 3.0 | 117.7 | Green | 147.5 |
| Device Exam- ple 12 | 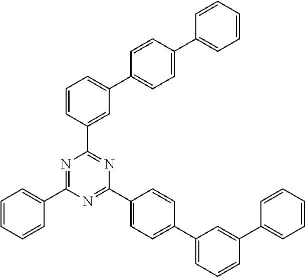 [SMILES: c1ccc(-c2ccc(-c3cccc(-c4nc(-c5ccccc5)nc(-c5ccc(-c6cccc(-c7ccccc7)c6)cc5)n4)c3)cc2)cc1] C-376 | 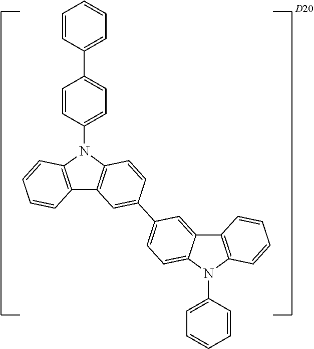 H2-2-D20 | 3.0 | 117.0 | Green | 156.7 |
| Device Exam- ple 13 | 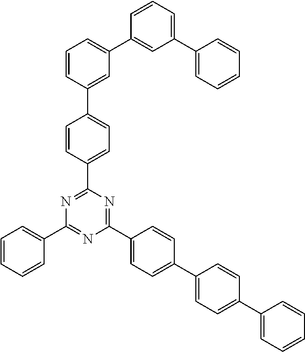 [SMILES: c1ccc(-c2ccc(-c3ccc(-c4nc(-c5ccccc5)nc(-c5ccc(-c6cccc(-c7cccc(-c8ccccc8)c7)c6)cc5)n4)cc3)cc2)cc1] C-377 | 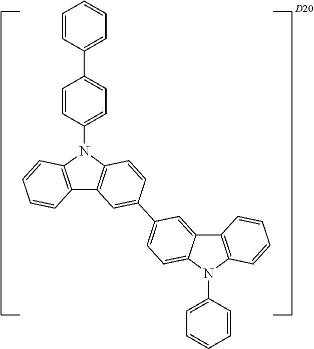 H2-2-D20 | 2.9 | 113.7 | Green | 95.0 |
| Com- parative Exam- ple 1 | 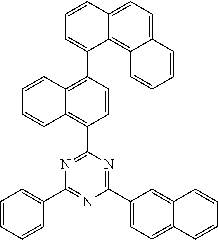 [SMILES: c1ccc(-c2nc(-c3ccc4ccccc4c3)nc(-c3ccc(-c4cccc5ccc6ccccc6c45)c4ccccc34)n2)cc1] C-ref | 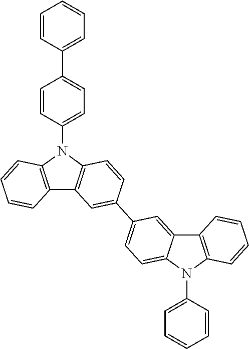 [SMILES: c1ccc(-c2ccc(-n3c4ccccc4c4cc(-c5ccc6c(c5)c5ccccc5n6-c5ccccc5)ccc43)cc2)cc1] H2-147 | 3.9 | 7.6 | Green | — |

From Table 1 above, it can be confirmed that the OLEDs comprising a plurality of host materials according to the present disclosure (Device Examples 1 to 13) exhibit excellent lifespan properties as well as lower driving voltage and higher luminous efficiency compared to the OLED comprising a conventional combination of hosts (Comparative Example 1). It was not possible to measure the lifespan of the OLED according to Comparative Example 1 due to its low efficiency.

The lifespan of a green light-emitting OLED is generally shorter than that of a red light-emitting OLED. In order to improve lifespan properties in green light-emitting OLEDs, the present disclosure used compounds into which deuterated moieties are introduced. Although not limited by theory, when an organic electroluminescent compound is substituted with deuterium, the zero point vibration energy of the compound is lowered, thereby increasing the bond dissociation energy (BDE) of the compound and thus increasing the stability of the compound.

### Device Examples 14 to 16: Producing an OLED Comprising a Compound According to the Present Disclosure as a Single Host Material

OLEDs were produced in the same manner as in Device Example 1, except that the compound shown in Table 2 below was used alone as a single host of a light-emitting layer.

### Device Examples 17 to 25: Producing an OLED Comprising a Compound According to the Present Disclosure as a Single Host Material

OLEDs were produced in the same manner as in Device Example 1, except that the compound shown in Table 2 below alone as a single host of a light-emitting layer and compound D-150 as a dopant were used.

### Comparative Examples 2 to 4: Producing an OLED Comprising a Conventional Compound as a Single Host Material

OLEDs were produced in the same manner as in Device Example 1, except that the compound shown in Table 2 below was used alone as a host of a light-emitting layer.

The driving voltage and light-emitting color at a luminance of 1,000 nit, and the time taken for reduction of luminance from 100% to 80% (lifespan: T80) at a luminance of 60,000 nit of the OLEDs produced in Device Examples 14 to 25 and Comparative Examples 2 to 4 are provided in Table 2 below.

TABLE 2

| | | Driving | | |
|---|---|---|---|---|
| | | Voltage | Light-Emitting | Lifespan |
| | Host Compound | (V) | Color | (T80, hr) |
| Device Example 14 | 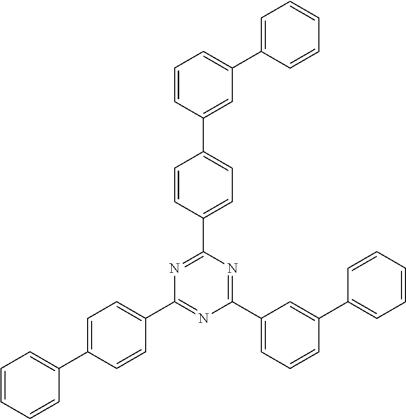 [SMILES: c1ccc(-c2ccc(-c3nc(-c4ccc(-c5cccc(-c6ccccc6)c5)cc4)nc(-c4cccc(-c5ccccc5)c4)n3)cc2)cc1] C-28 | 2.7 | Green | 16.7 |
| Device Example 15 | 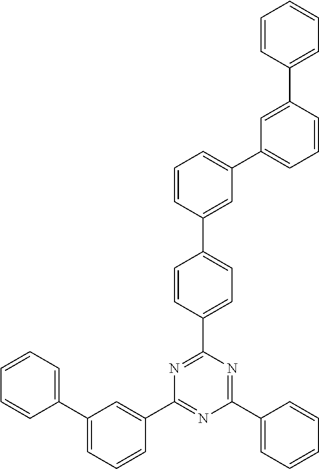 [SMILES: c1ccc(-c2cccc(-c3cccc(-c4ccc(-c5nc(-c6ccccc6)nc(-c6cccc(-c7ccccc7)c6)n5)cc4)c3)c2)cc1] C-198 | 2.8 | Green | 24.6 |
| Device Example 16 | 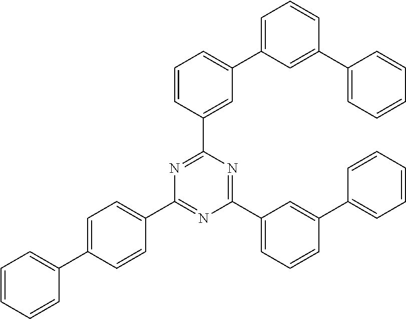 [SMILES: c1ccc(-c2ccc(-c3nc(-c4cccc(-c5ccccc5)c4)nc(-c4cccc(-c5cccc(-c6ccccc6)c5)c4)n3)cc2)cc1] C-109 | 2.7 | Green | 26.0 |
| Device Example 17 | 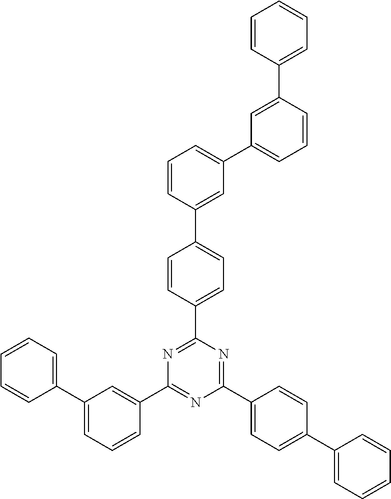 [SMILES: c1ccc(-c2ccc(-c3nc(-c4ccc(-c5cccc(-c6cccc(-c7ccccc7)c6)c5)cc4)nc(-c4cccc(-c5ccccc5)c4)n3)cc2)cc1] C-199 | 2.7 | Green | 130.9 |
| Device Example 18 | 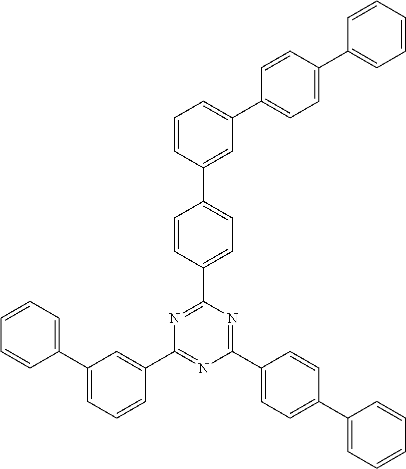 [SMILES: c1ccc(-c2ccc(-c3cccc(-c4ccc(-c5nc(-c6ccc(-c7ccccc7)cc6)nc(-c6cccc(-c7ccccc7)c6)n5)cc4)c3)cc2)cc1] C-370 | 2.6 | Green | 103.6 |
| Device Example 19 | 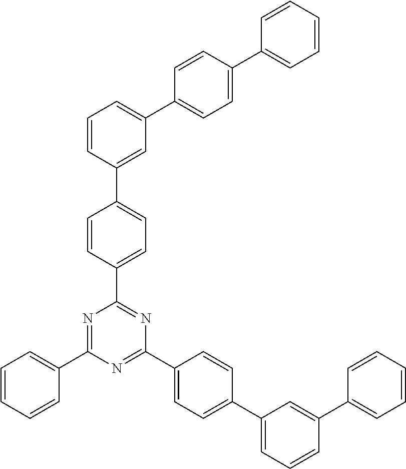 [SMILES: c1ccc(-c2ccc(-c3cccc(-c4ccc(-c5nc(-c6ccccc6)nc(-c6ccc(-c7cccc(-c8ccccc8)c7)cc6)n5)cc4)c3)cc2)cc1] C-371 | 2.7 | Green | 92.0 |
| Device Example 20 | 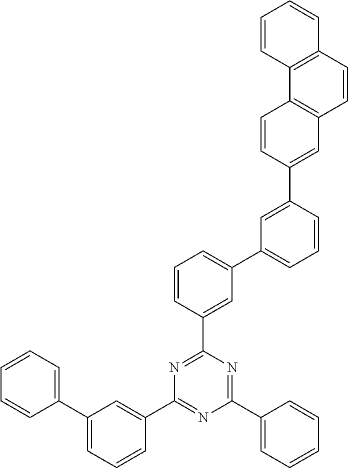 [SMILES: c1ccc(-c2cccc(-c3nc(-c4ccccc4)nc(-c4cccc(-c5cccc(-c6ccc7c(ccc8ccccc87)c6)c5)c4)n3)c2)cc1] C-372 | 2.8 | Green | 131.8 |
| Device Example 21 | 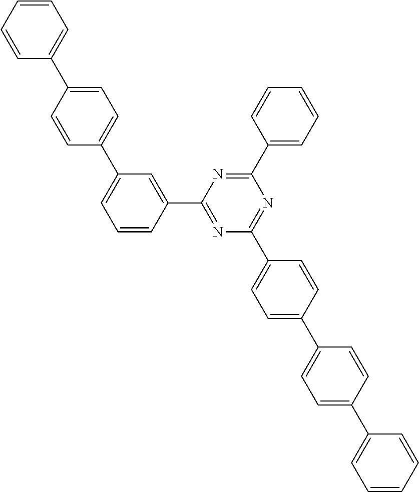 [SMILES: c1ccc(-c2ccc(-c3ccc(-c4nc(-c5ccccc5)nc(-c5cccc(-c6ccc(-c7ccccc7)cc6)c5)n4)cc3)cc2)cc1] C-373 | 3.0 | Green | 16.2 |
| Device Example 22 | 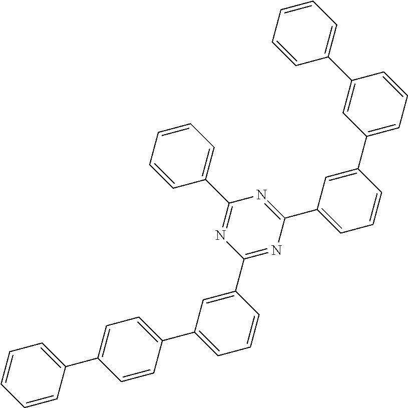 [SMILES: c1ccc(-c2ccc(-c3cccc(-c4nc(-c5ccccc5)nc(-c5cccc(-c6cccc(-c7ccccc7)c6)c5)n4)c3)cc2)cc1] C-374 | 3.0 | Green | 127.6 |
| Device Example 23 | 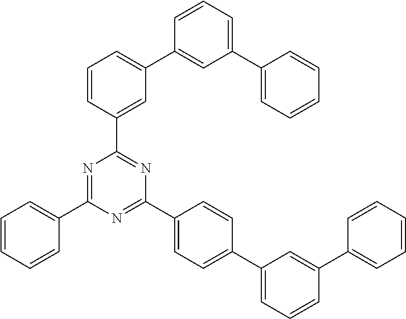 [SMILES: c1ccc(-c2cccc(-c3ccc(-c4nc(-c5ccccc5)nc(-c5cccc(-c6cccc(-c7ccccc7)c6)c5)n4)cc3)c2)cc1] C-375 | 2.9 | Green | 149.5 |
| Device Example 24 | 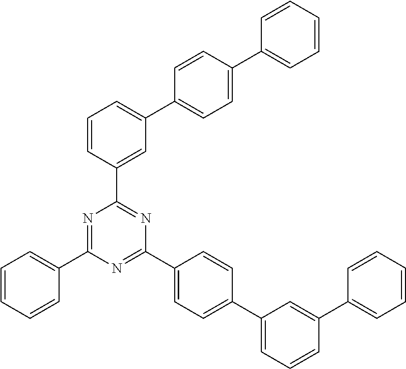 [SMILES: c1ccc(-c2ccc(-c3cccc(-c4nc(-c5ccccc5)nc(-c5ccc(-c6cccc(-c7ccccc7)c6)cc5)n4)c3)cc2)cc1] C-376 | 2.7 | Green | 132.8 |
| Device Example 25 | 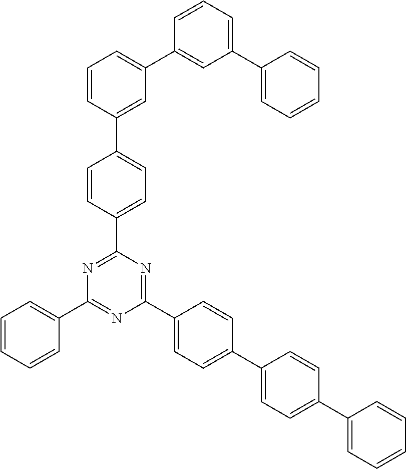 [SMILES: c1ccc(-c2ccc(-c3ccc(-c4nc(-c5ccccc5)nc(-c5ccc(-c6cccc(-c7cccc(-c8ccccc8)c7)c6)cc5)n4)cc3)cc2)cc1] C-377 | 2.6 | Green | 41.5 |
| Comparative Example 2 | 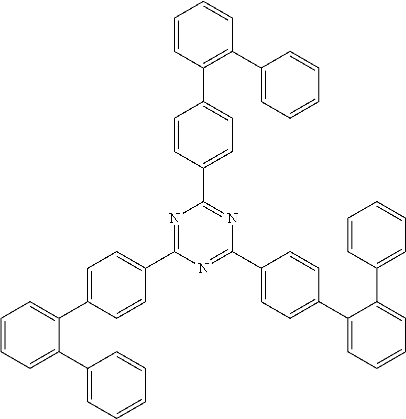 [SMILES: c1ccc(-c2ccccc2-c2ccc(-c3nc(-c4ccc(-c5ccccc5-c5ccccc5)cc4)nc(-c4ccc(-c5ccccc5-c5ccccc5)cc4)n3)cc2)cc1] C-274 | 2.6 | Green | 0.3 |
| Comparative Example 3 | 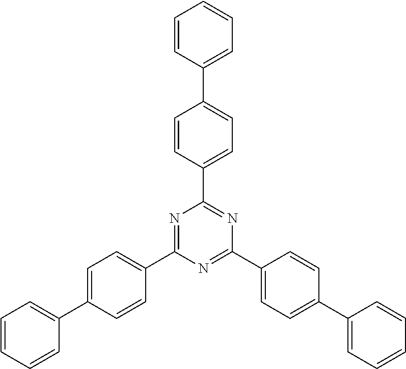 [SMILES: c1ccc(-c2ccc(-c3nc(-c4ccc(-c5ccccc5)cc4)nc(-c4ccc(-c5ccccc5)cc4)n3)cc2)cc1] C-266 | 2.7 | Green | 3.0 |
| Comparative Example 4 | 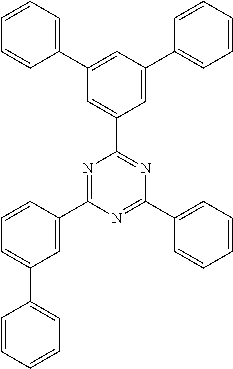 [SMILES: c1ccc(-c2cccc(-c3nc(-c4ccccc4)nc(-c4cc(-c5ccccc5)cc(-c5ccccc5)c4)n3)c2)cc1] C-304 | 2.9 | Green | 1.3 |

From Table 2 above, it can be confirmed that the OLEDs comprising a compound according to the present disclosure as a host material (Device Examples 14 to 25) exhibit excellent lifespan properties compared to the OLEDs comprising a conventional compound as a host material (Comparative Examples 2 to 4).

The compounds used in the Device Examples and the Comparative Examples are shown in Table 3 below.

TABLE 3

| | |
|---|---|
| Hole Injection Layer/Hole Transport Layer | 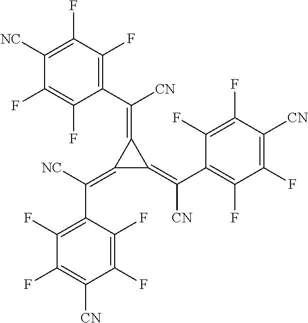 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] HI-1 |
| | 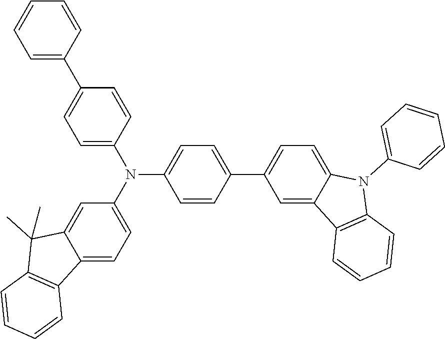 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] HT-1 |
| | 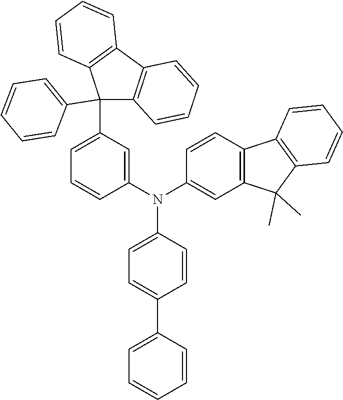 [SMILES: CC1(C)c2ccccc2-c2ccc(N(c3ccc(-c4ccccc4)cc3)c3cccc(C4(c5ccccc5)c5ccccc5-c5ccccc54)c3)cc21] HT-2 |
| Light-Emitting Layer | 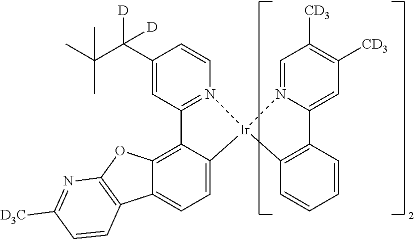 D-130 |
| | 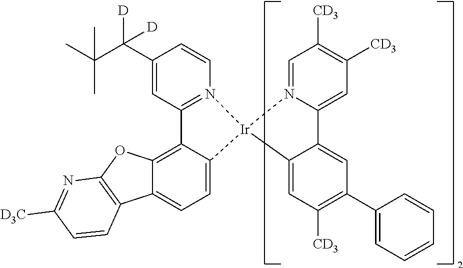 D-150 |
| Electron Transport Layer/ Electron Injection Layer | 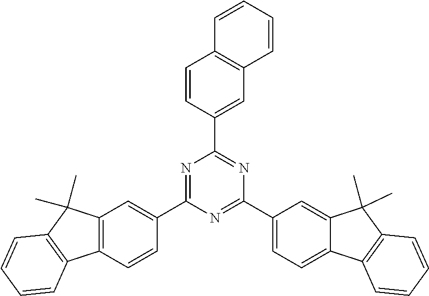 [SMILES: CC1(C)c2ccccc2-c2ccc(-c3nc(-c4ccc5c(c4)C(C)(C)c4ccccc4-5)nc(-c4ccc5ccccc5c4)n3)cc21] ETL-1 |
| | 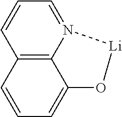 EIL-1 |

1. A plurality of host materials comprising at least one first host compound and at least one second host compound, wherein the first host compound is represented by the following formula 1, and the second host compound is represented by the following formula 2, and wherein at least one of the first host compound and the second host compound contains deuterium: 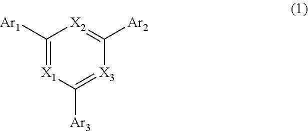
in formula 1,
X₁ to X₃ each independently represent N or CRₐ; with the proviso that at least two of X₁ to X₃ are N;
Rₐ represents hydrogen or deuterium; and
Ar₁ to Ar₃ each independently represent a (C6-C30)aryl unsubstituted or substituted with at least one of deuterium and a (C6-C30)aryl(s); 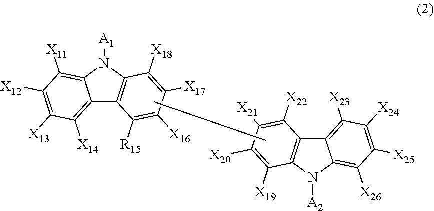
in formula 2,
A₁ and A₂ each independently represent a substituted or unsubstituted (C6-C30)aryl, a substituted or unsubstituted dibenzofuranyl, a substituted or unsubstituted dibenzothiophenyl, or a substituted or unsubstituted carbazolyl;
any one of X₁₅ to X₁₈ and any one of X₁₉ to X₂₂ are linked to each other to form a single bond; and
X₁₁ to X₁₄, X₂₃ to X₂₆, and X₁₅ to X₂₂ which do not form a single bond each independently represent hydrogen, deuterium, a substituted or unsubstituted (C6-C30)aryl, or a substituted or unsubstituted (3- to 30-membered)heteroaryl; or may be linked to an adjacent substituent to form a ring(s).

2. The plurality of host materials according to claim 1, wherein the substituents of the substituted aryl, the substituted heteroaryl, the substituted dibenzofuranyl, the substituted dibenzothiophenyl, and the substituted carbazolyl each independently are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of deuterium and a (C6-C30)aryl(s); a (C6-C30)aryl unsubstituted or substituted with at least one of deuterium and a (C6-C30)aryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30)aliphatic ring(s) and a (C6-C30)aromatic ring(s); an amino; a mono- or di-(C1-C30)alkylamino; a mono- or di-(C2-C30)alkenylamino; a (C1-C30)alkyl(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a (C6-C30)arylphosphine; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein Ar₁ to Ar₃ of formula 1 are each independently a phenyl unsubstituted or substituted with deuterium, a biphenyl unsubstituted or substituted with deuterium, a terphenyl unsubstituted or substituted with deuterium, a quaterphenyl unsubstituted or substituted with deuterium, a naphthyl unsubstituted or substituted with deuterium, a phenylnaphthyl unsubstituted or substituted with deuterium, a naphthylphenyl unsubstituted or substituted with deuterium, a triphenylenyl unsubstituted or substituted with deuterium, a phenanthrenyl unsubstituted or substituted with deuterium, or a combination thereof.

4. The plurality of host materials according to claim 1, wherein Ar₁ to Ar₃ of formula 1 are all different from each other.

5. The plurality of host materials according to claim 1, wherein formula 1 does not contain deuterium, and formula 2 contains deuterium.

6. The plurality of host materials according to claim 1, wherein the compound represented by formula 1 has a deuterium substitution rate of 30% to 100%.

7. The plurality of host materials according to claim 1, wherein at least one of X₁₁, X₁₈, X₁₉, and X₂₆ in formula 2 is deuterium,

8. The plurality of host materials according to claim 1, wherein the compound represented by formula 2 has a deuterium substitution rate of 40% to 100%.

9. The plurality of host materials according to claim 1, wherein the deuterium substitution rate of X₁₁ to X₂₆ in formula 2 is 25% to 100%.

10. The plurality of host materials according to claim 1, wherein formula 2 is represented by any one of the following formulas 2-1 to 2-8: 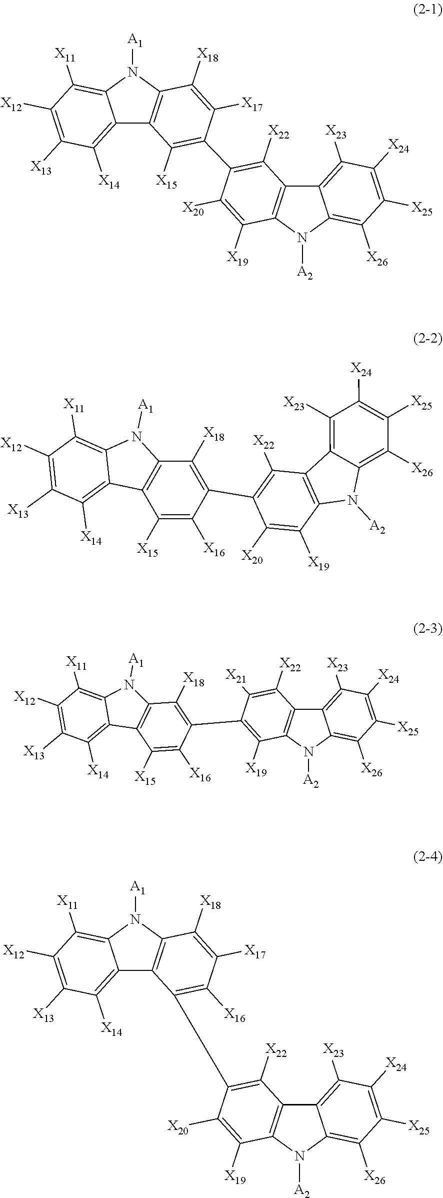 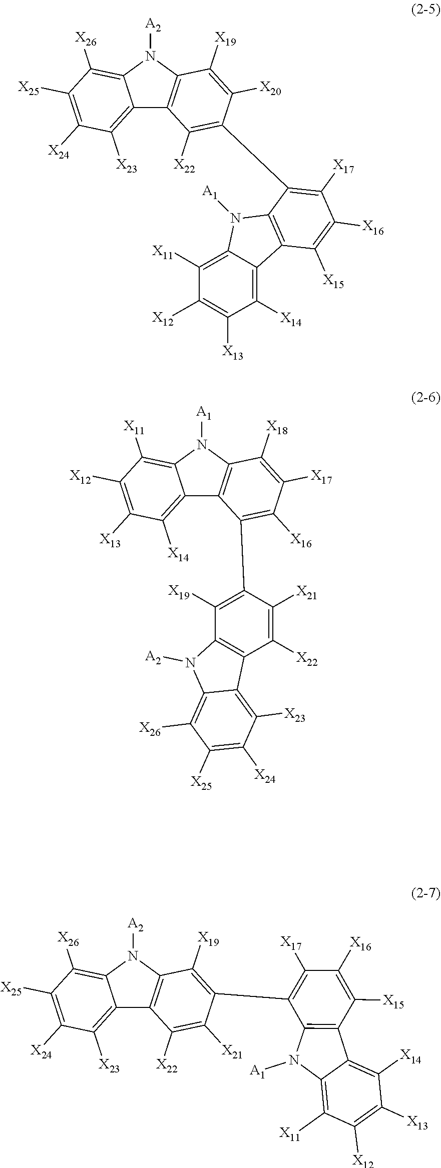 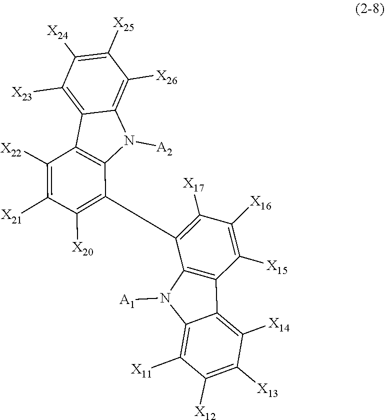
in formulas 2-1 to 2-8,
A₁, A₂, and X₁₁ to X₂₆ are as defined in claim 1.

11. The plurality of host materials according to claim 1, wherein A₁ and A₂ of formula 2 are each independently a phenyl unsubstituted or substituted with deuterium; a biphenyl unsubstituted or substituted with deuterium; a terphenyl unsubstituted or substituted with deuterium; a naphthyl unsubstituted or substituted with deuterium; a fluorenyl unsubstituted or substituted with at least one of deuterium, a (C1-C30)alkyl(s), and a (C6-C30)aryl(s); a benzofluorenyl unsubstituted or substituted with at least one of deuterium, a (C1-C30)alkyl(s), and a (C6-C30)aryl(s); a triphenylenyl unsubstituted or substituted with deuterium; a fluoranthenyl unsubstituted or substituted with deuterium; a phenanthrenyl unsubstituted or substituted with deuterium; a dibenzofuranyl unsubstituted or substituted with deuterium; a carbazolyl unsubstituted or substituted with deuterium; a dibenzothiophenyl unsubstituted or substituted with deuterium; or a combination thereof.

12. The plurality of host materials according to claim 1, wherein the compound represented by formula 1 is at least one selected from the following compounds: 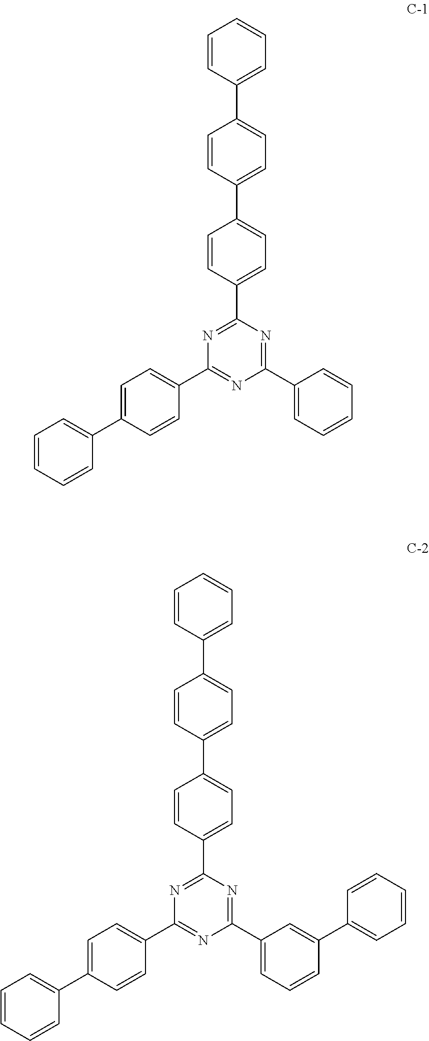 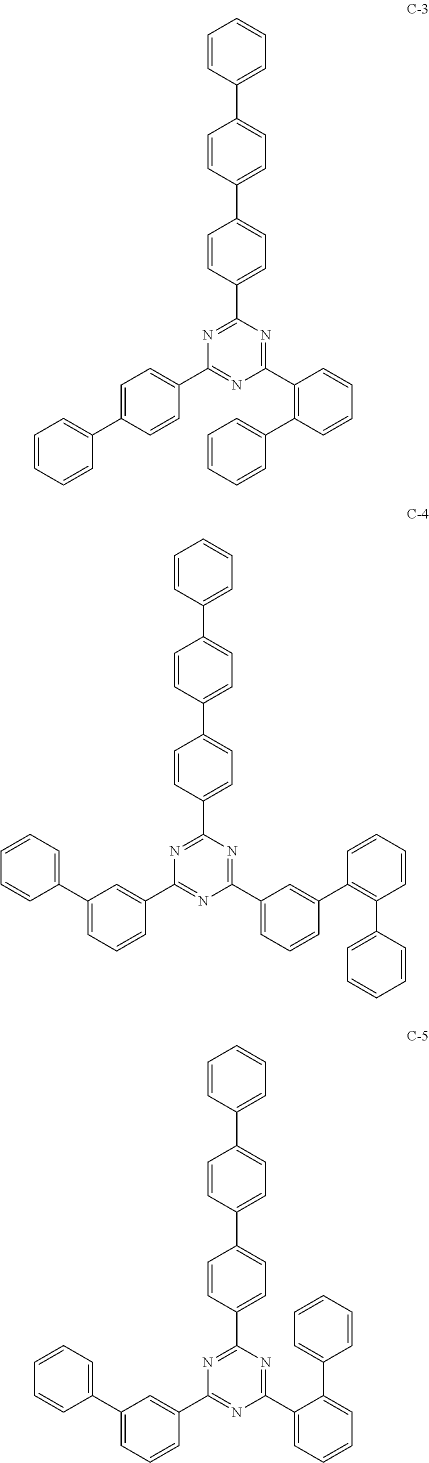 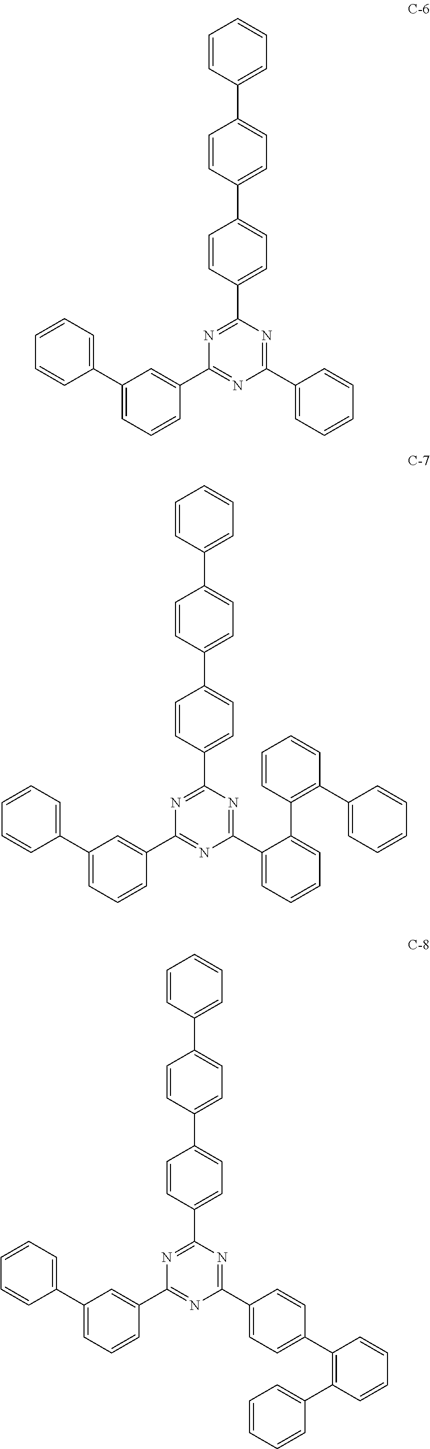 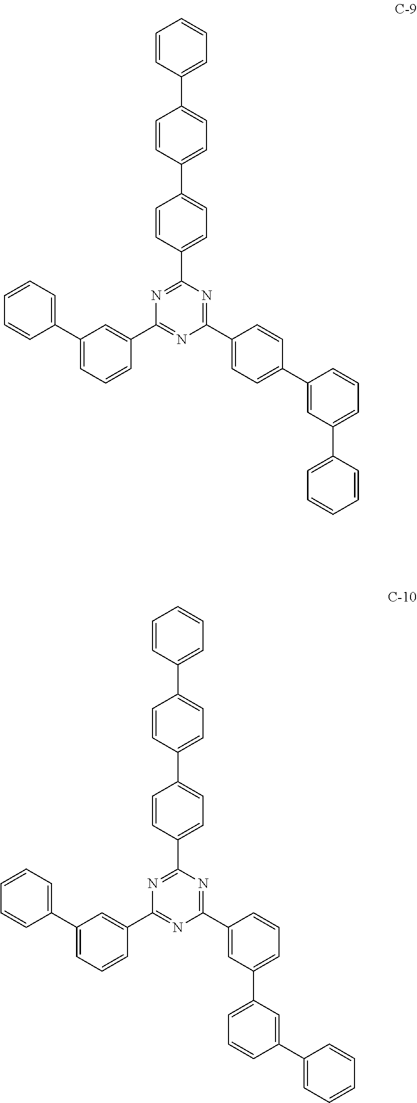 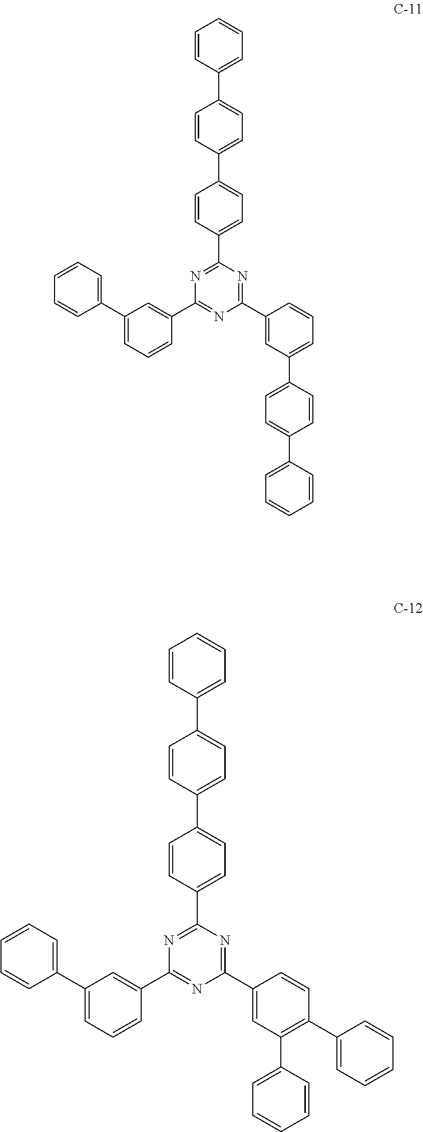 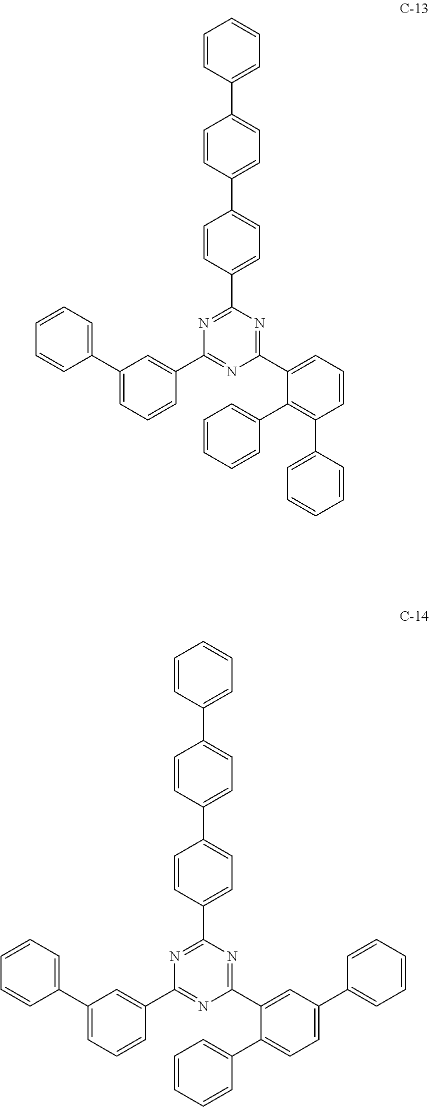 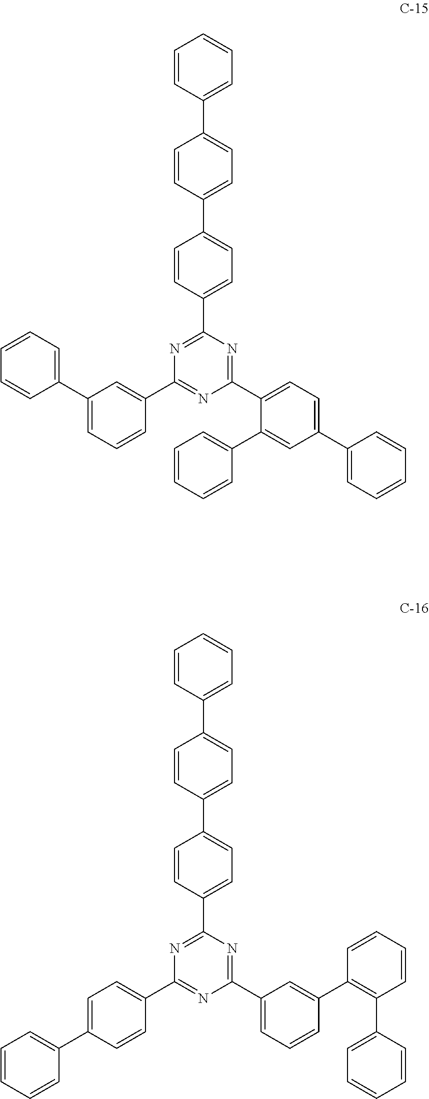 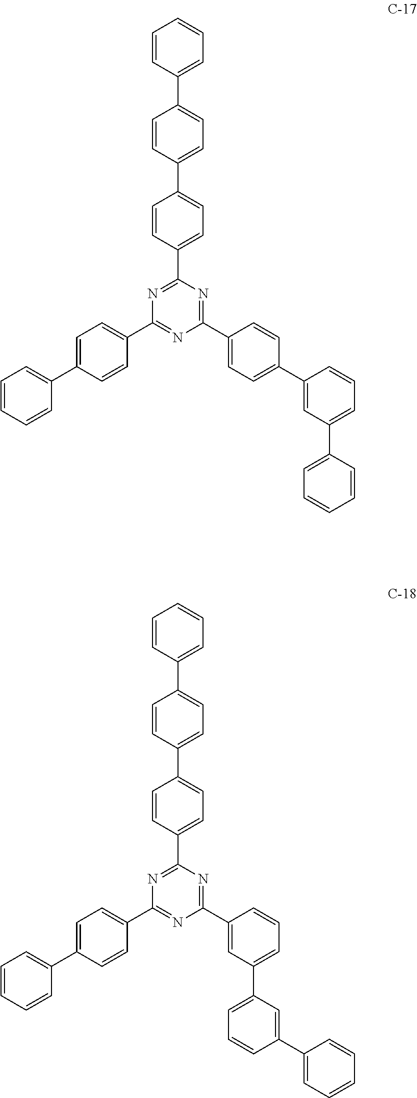 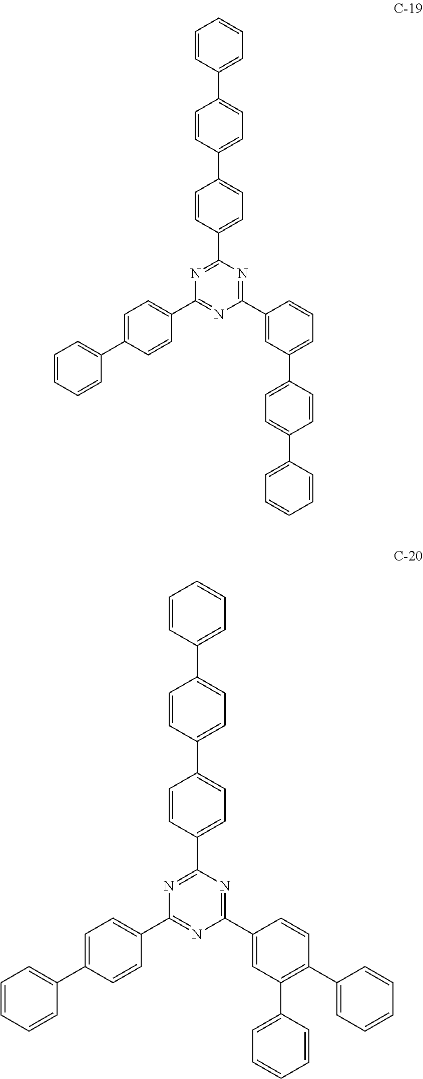 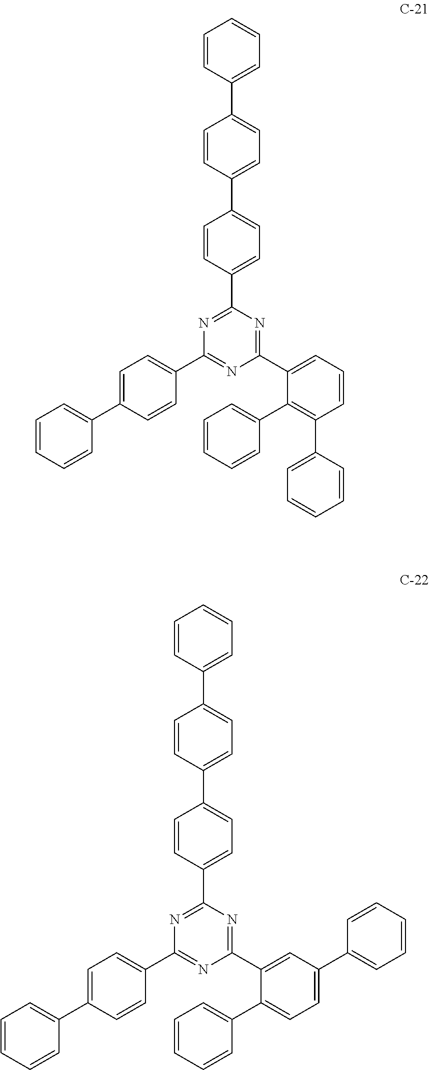 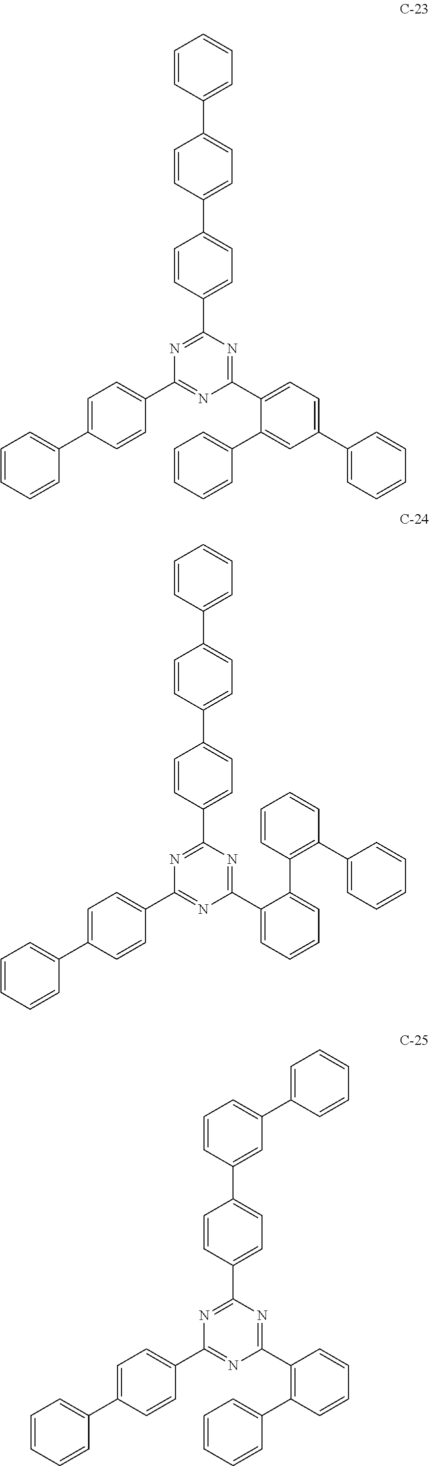 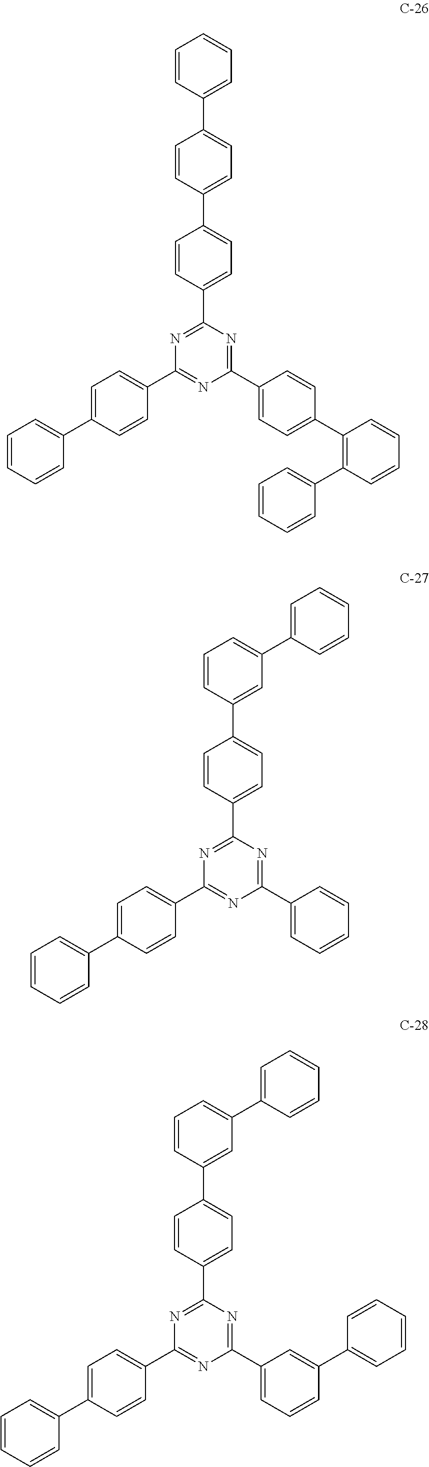 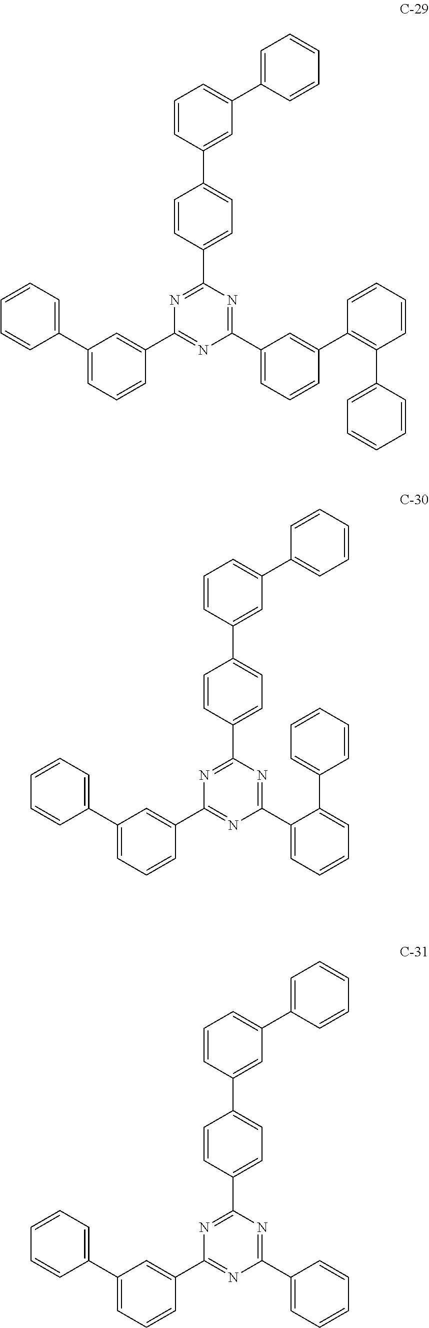 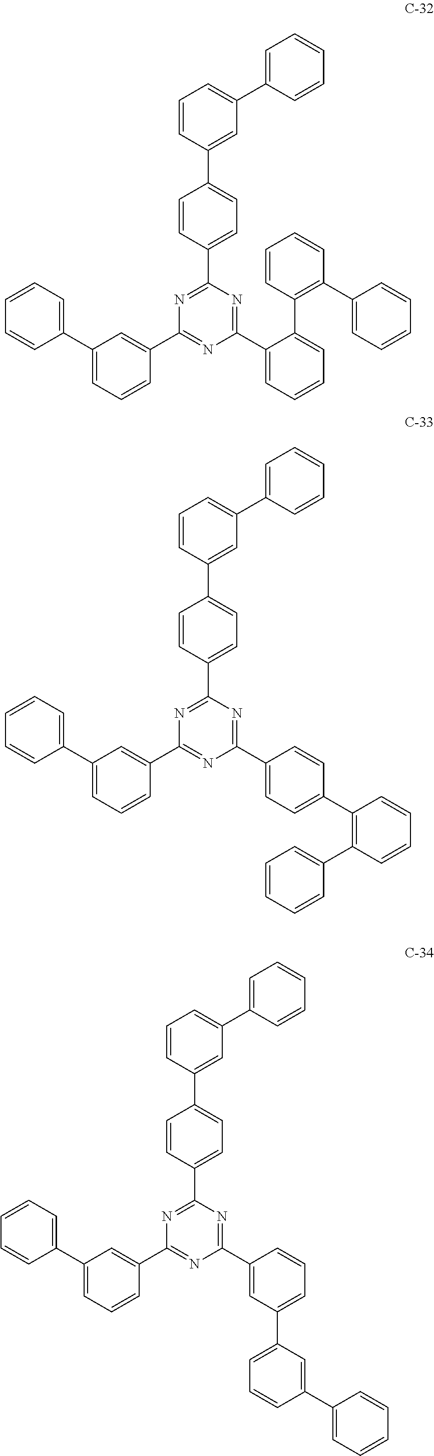 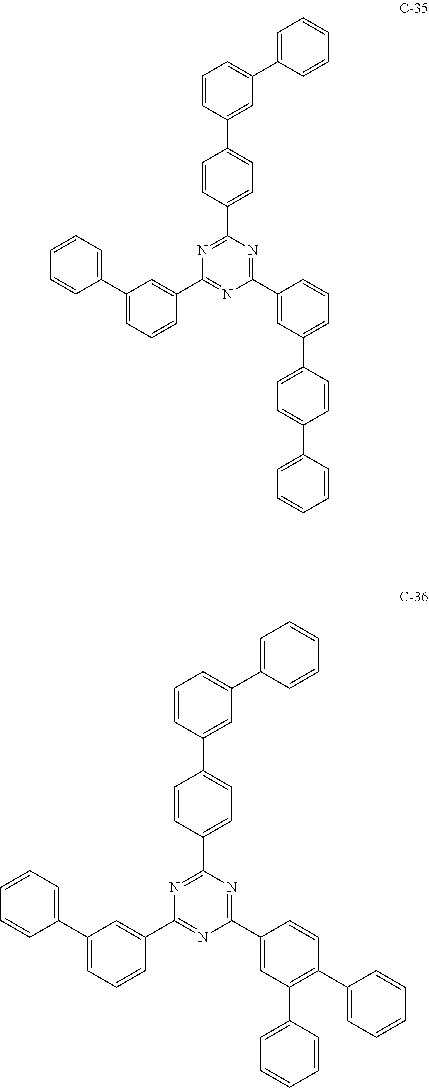 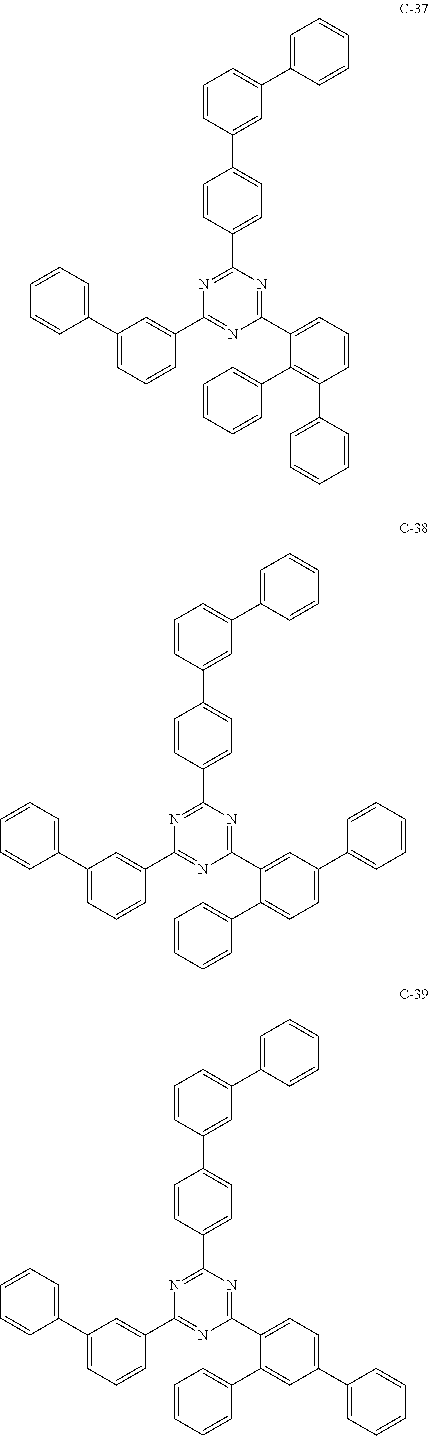 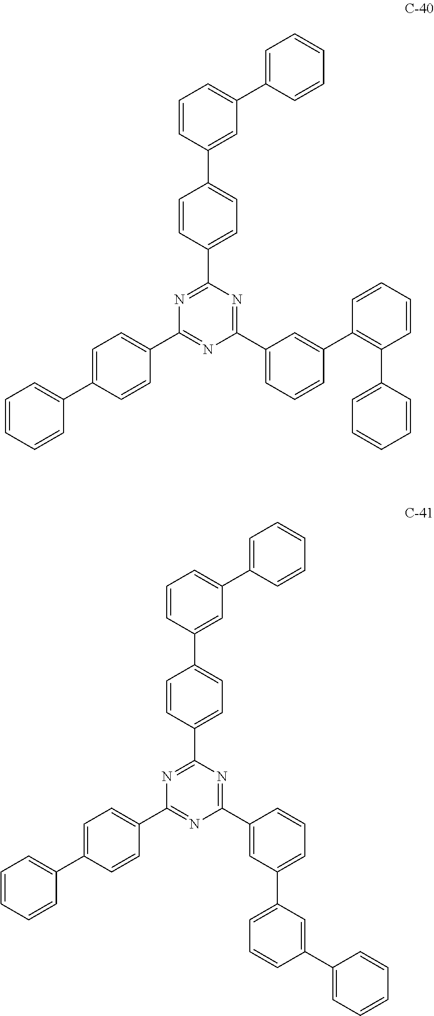 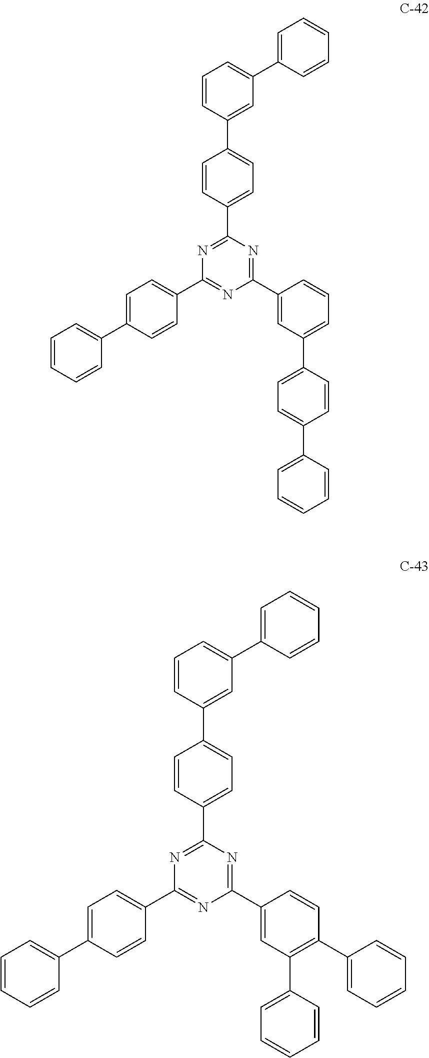 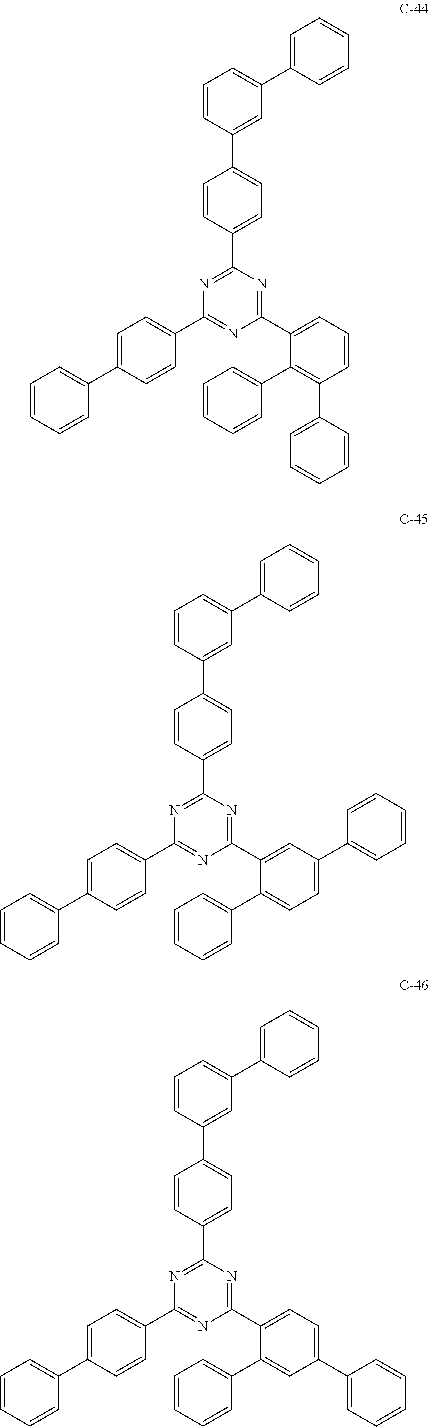 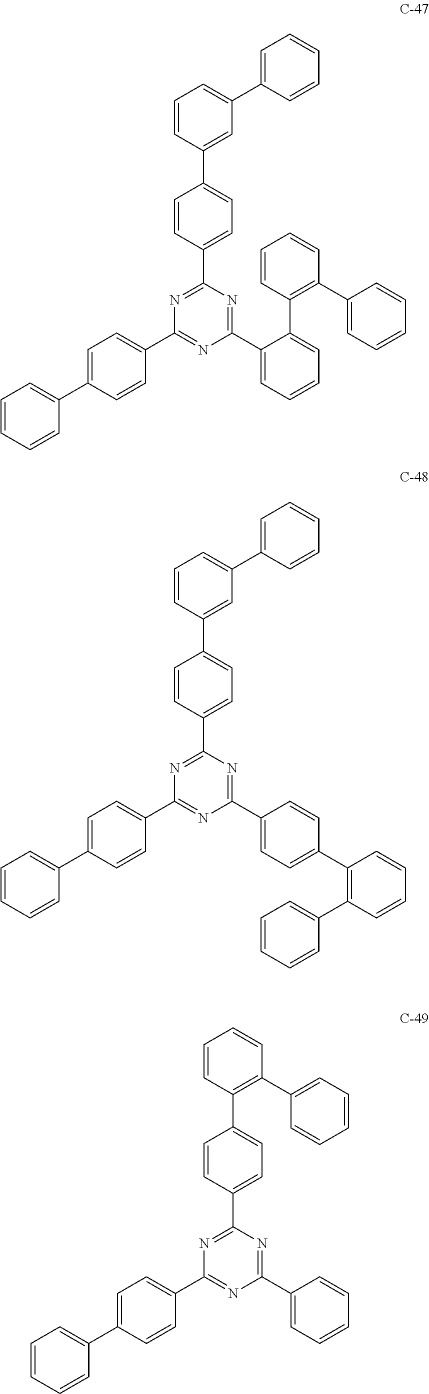 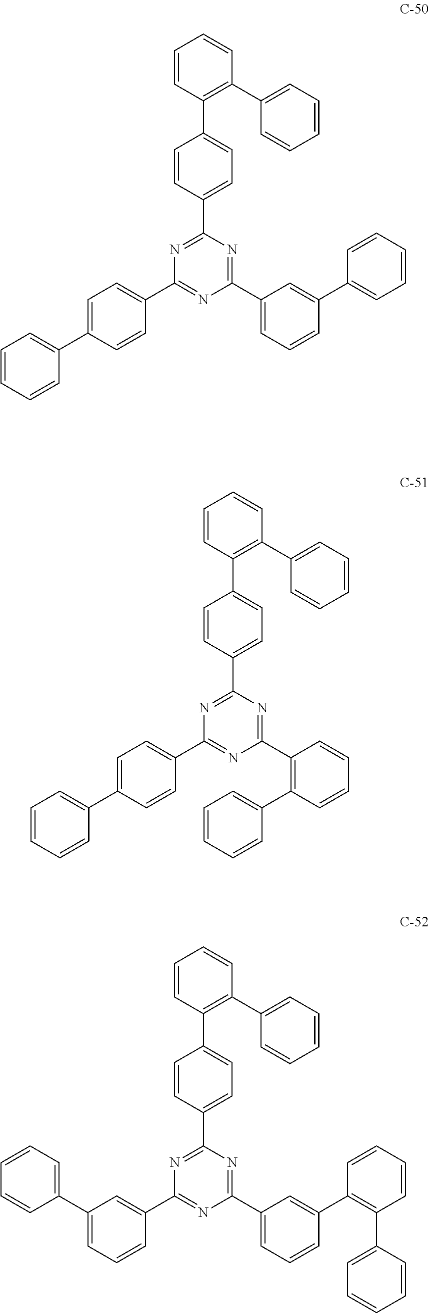 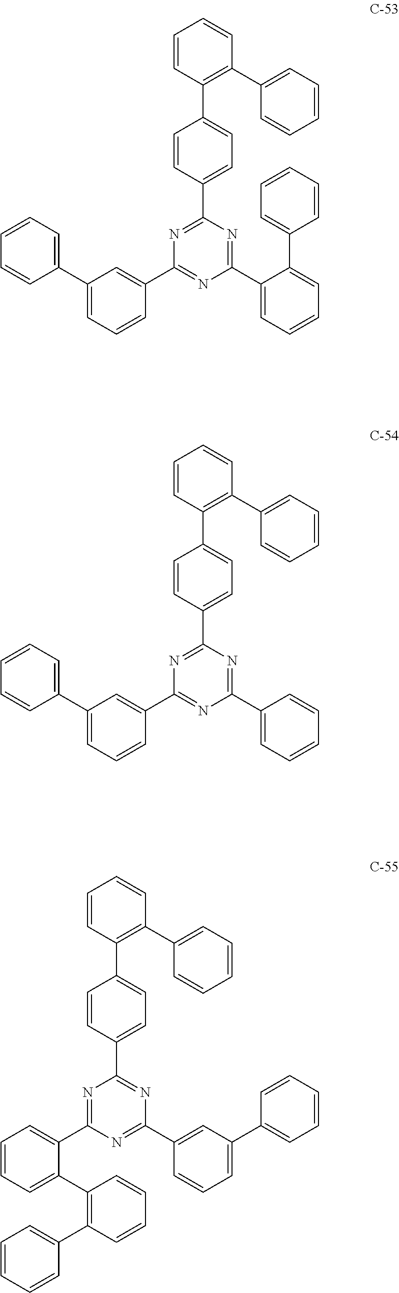 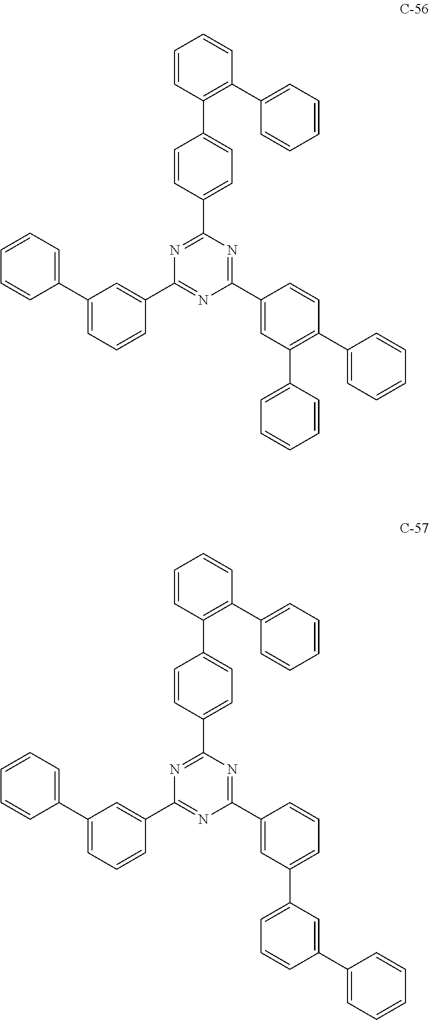 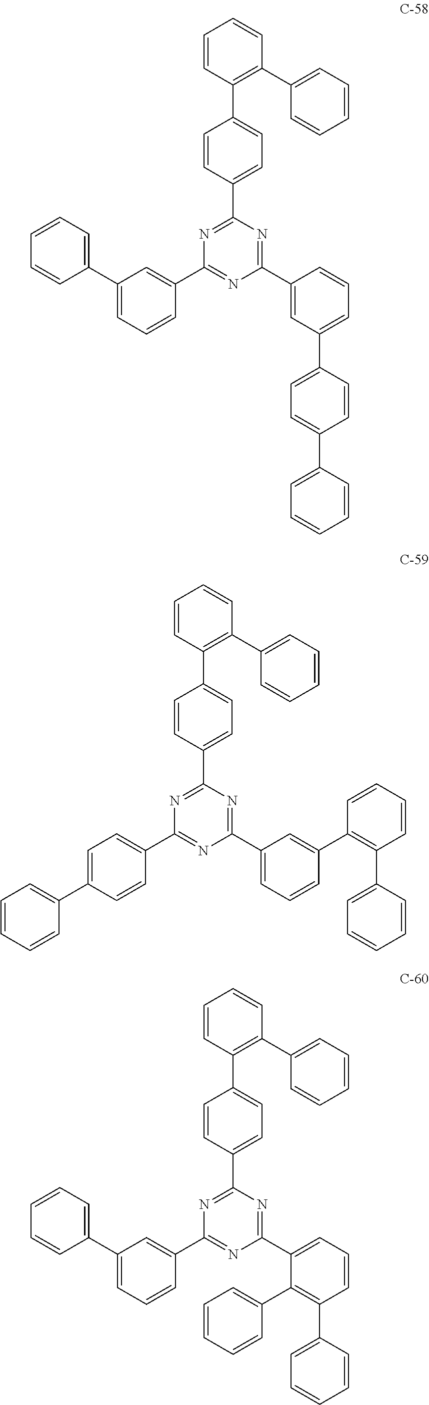 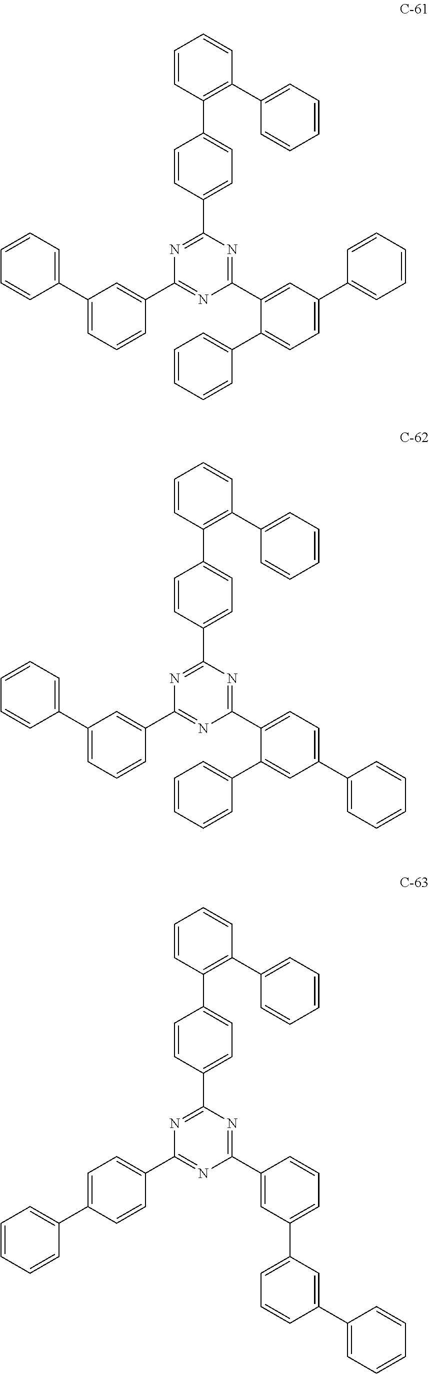 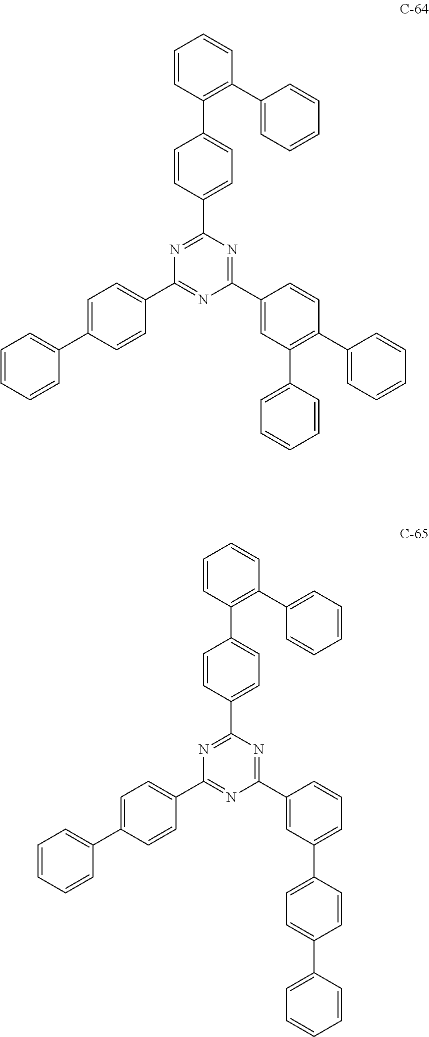 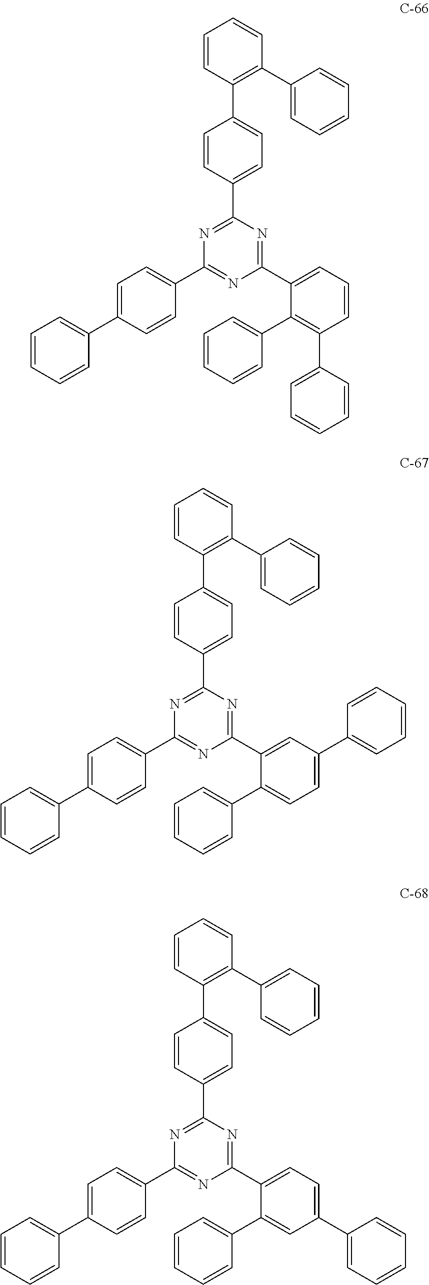 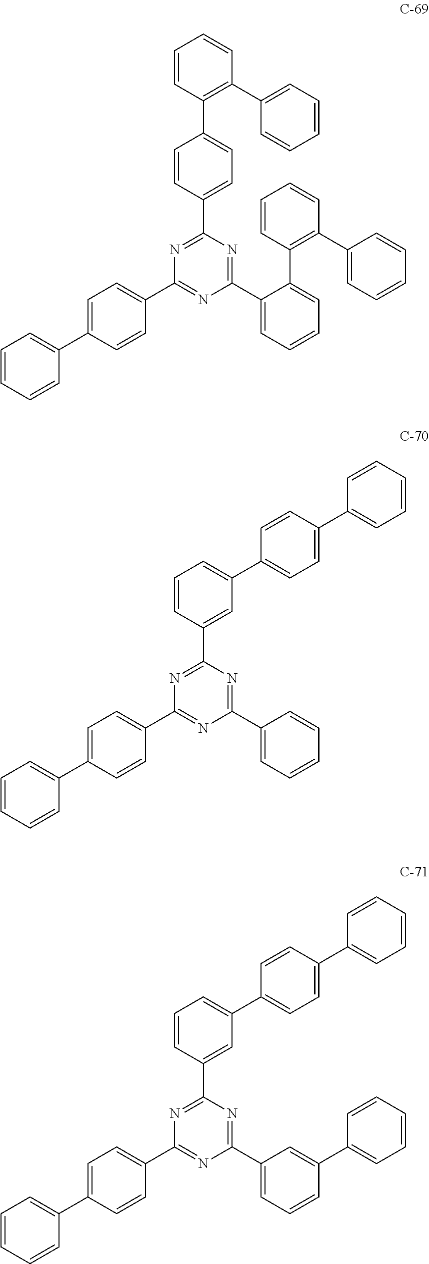 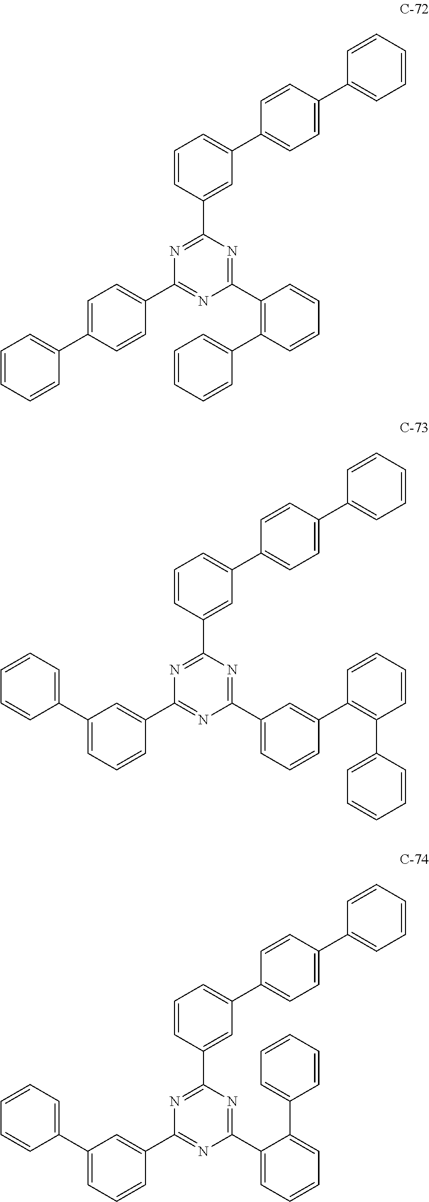 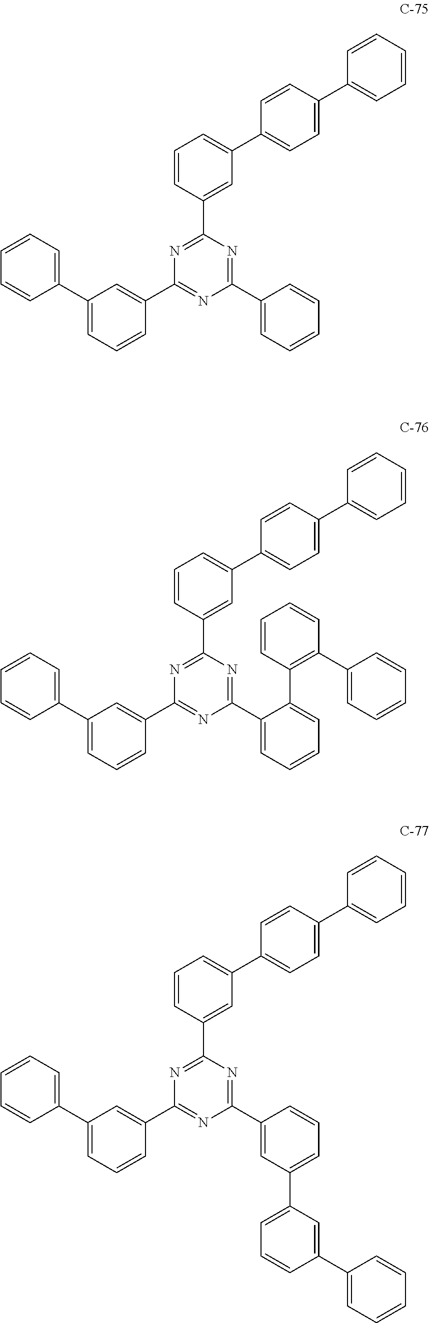 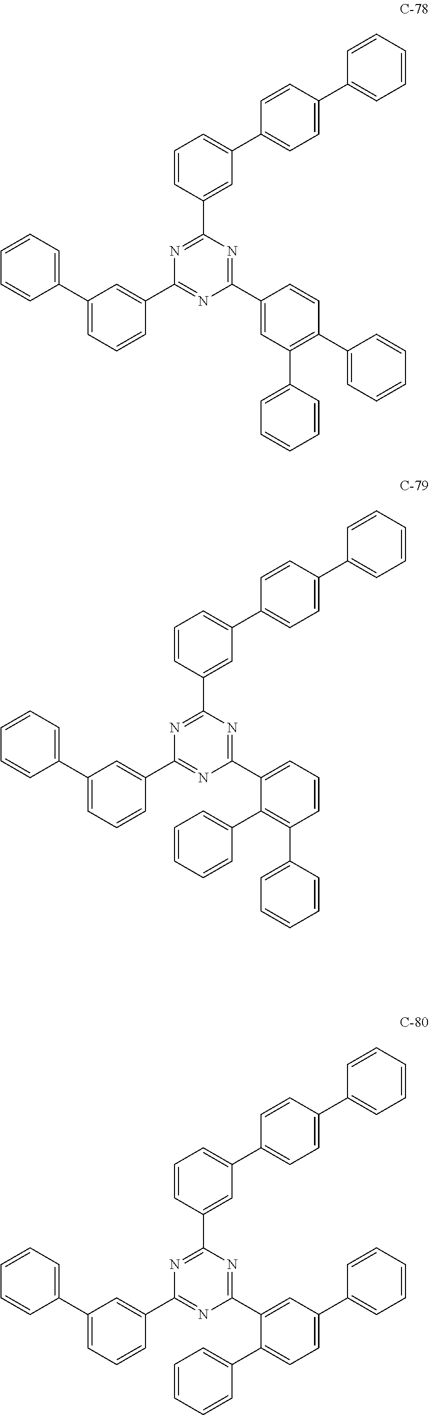 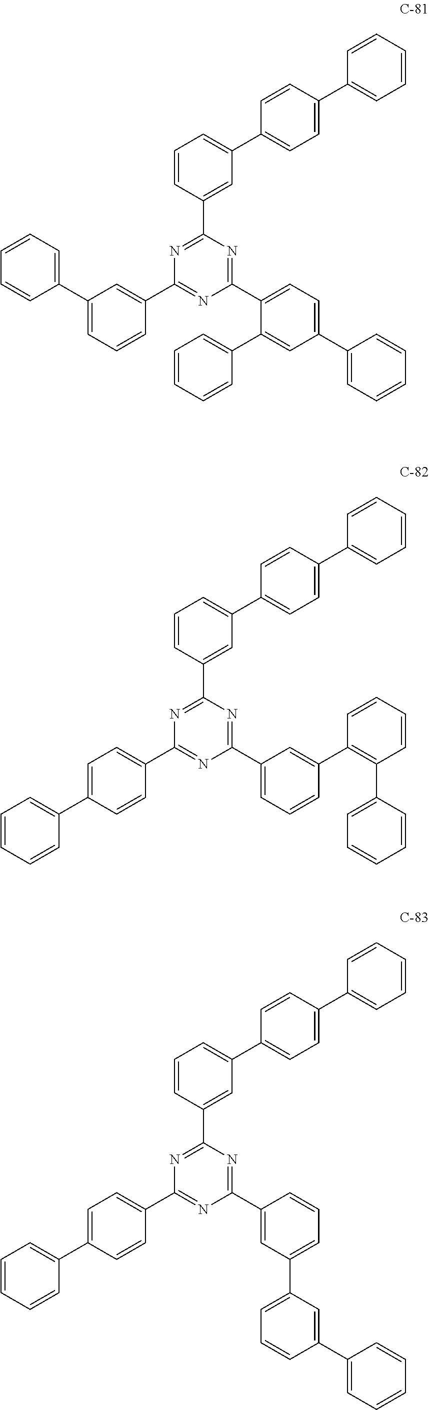 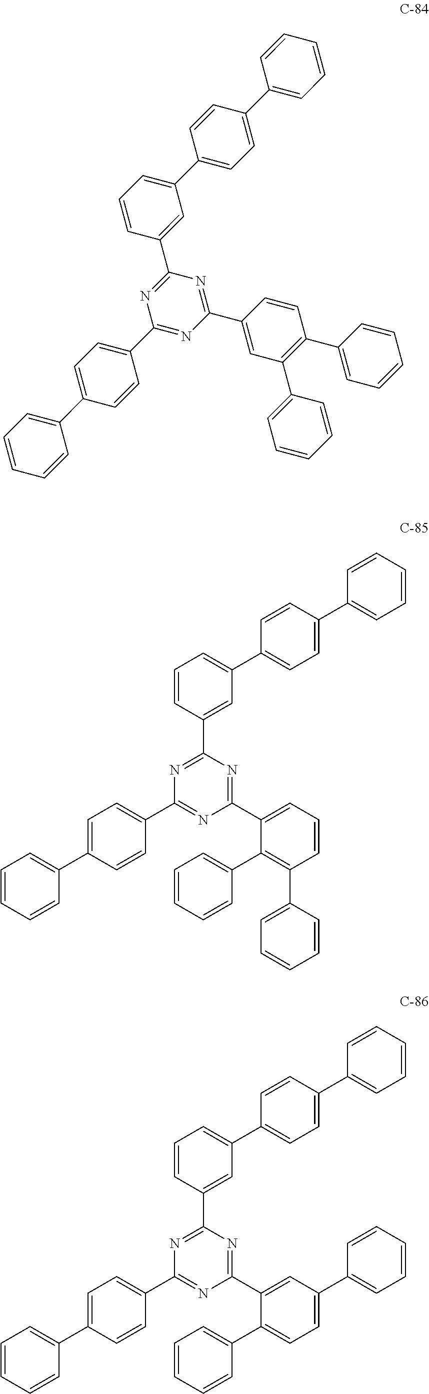 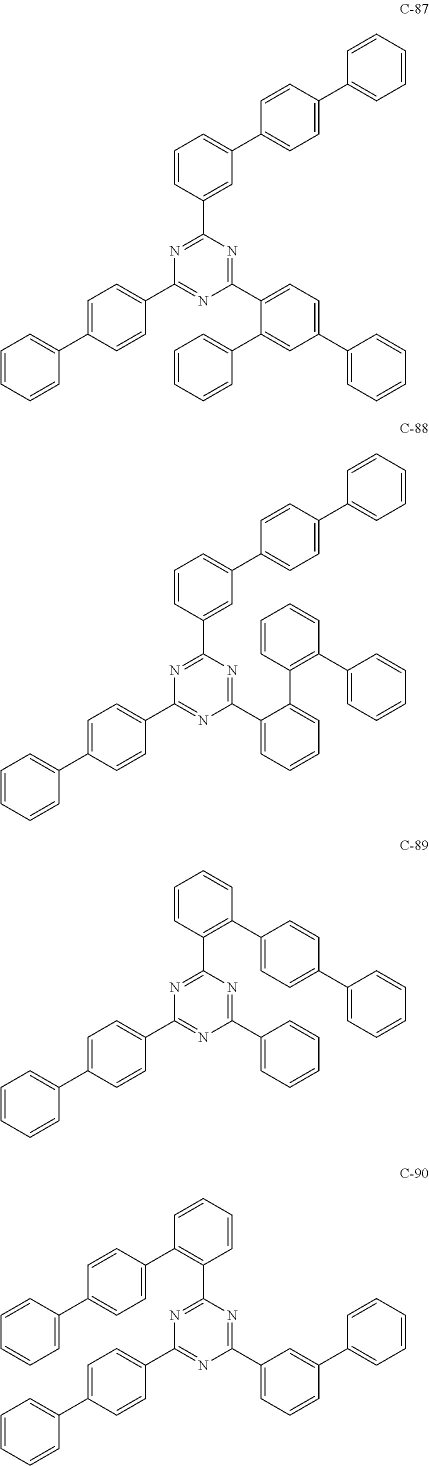 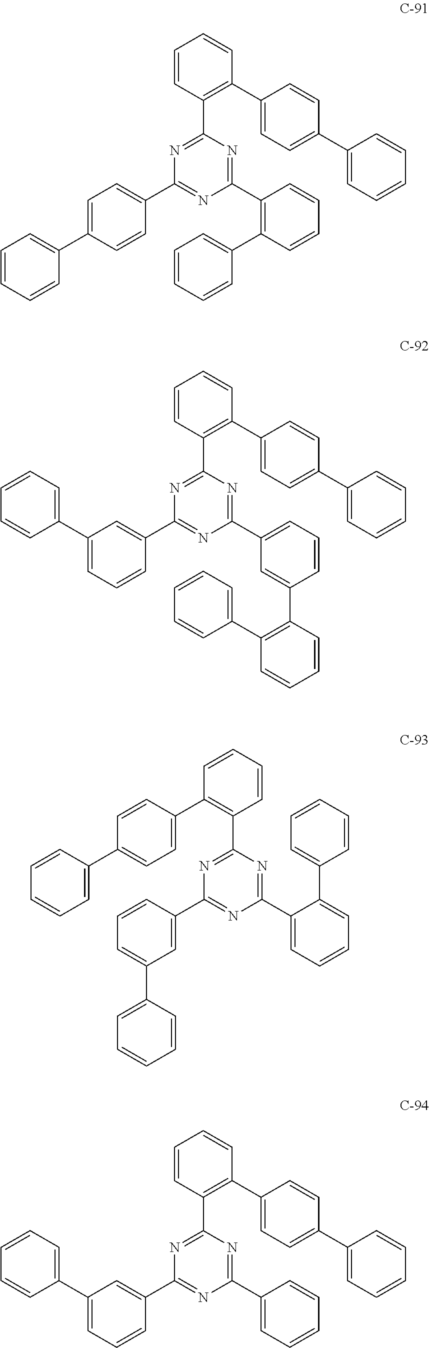 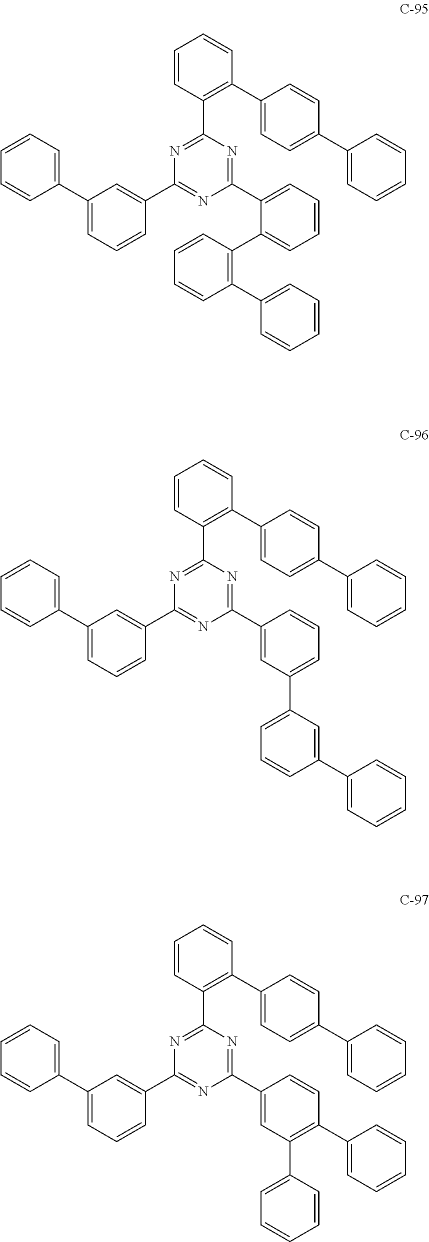 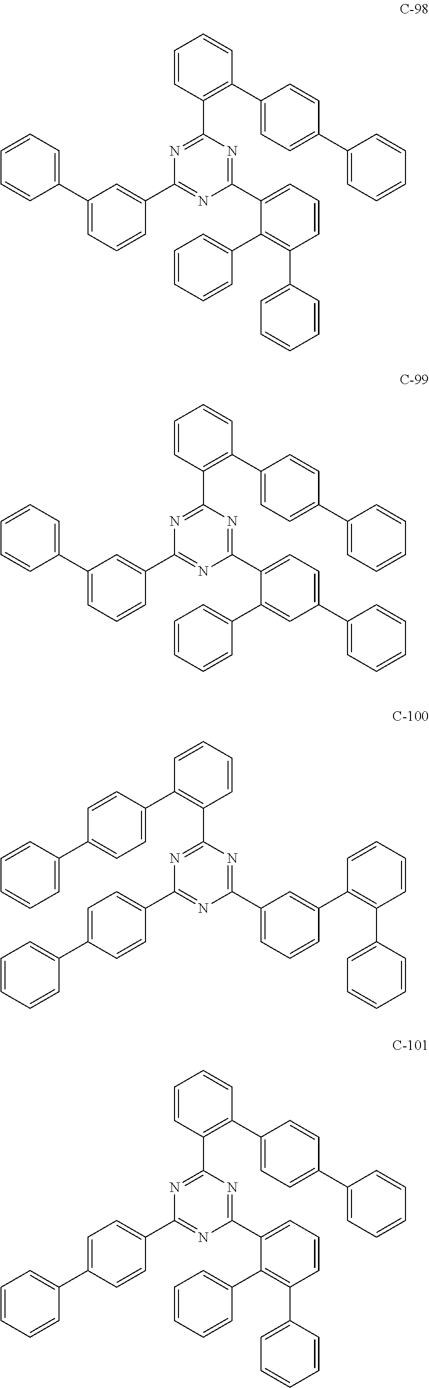 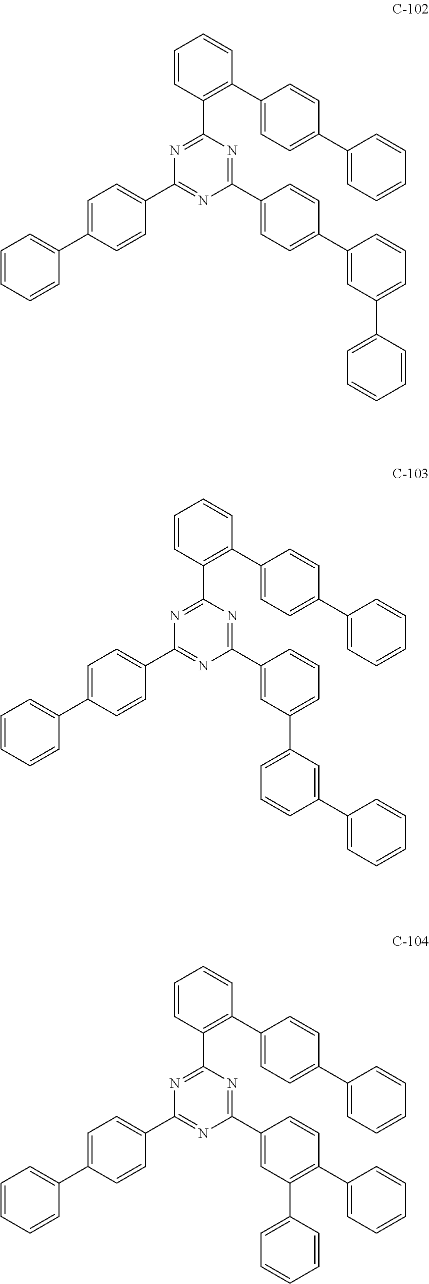 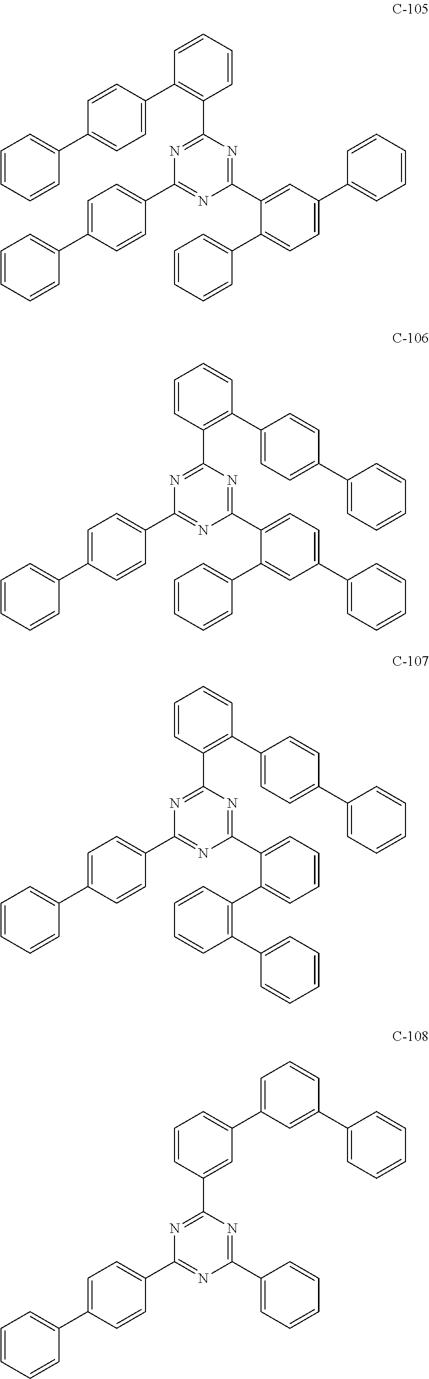 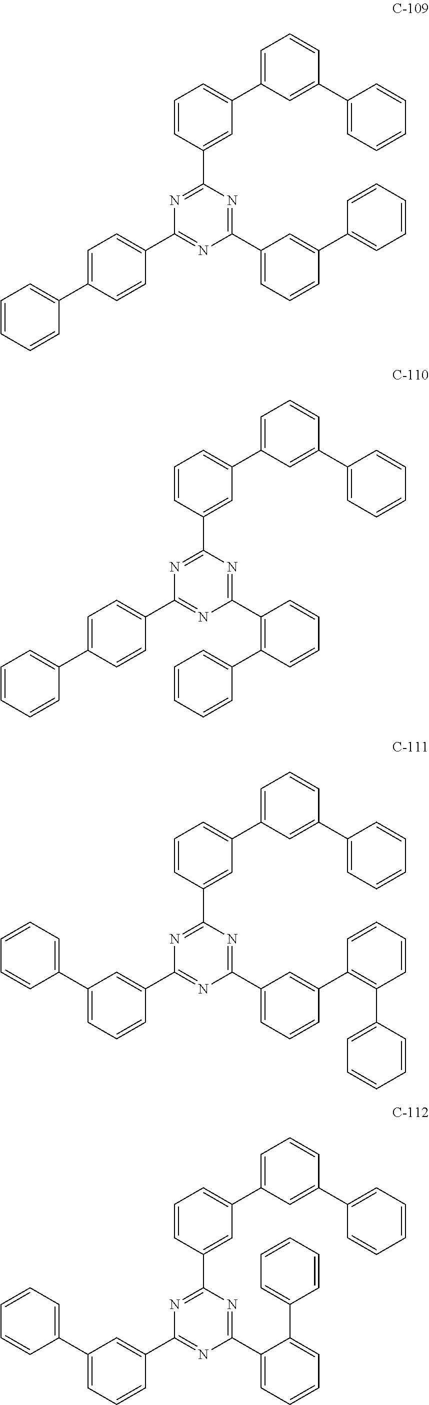 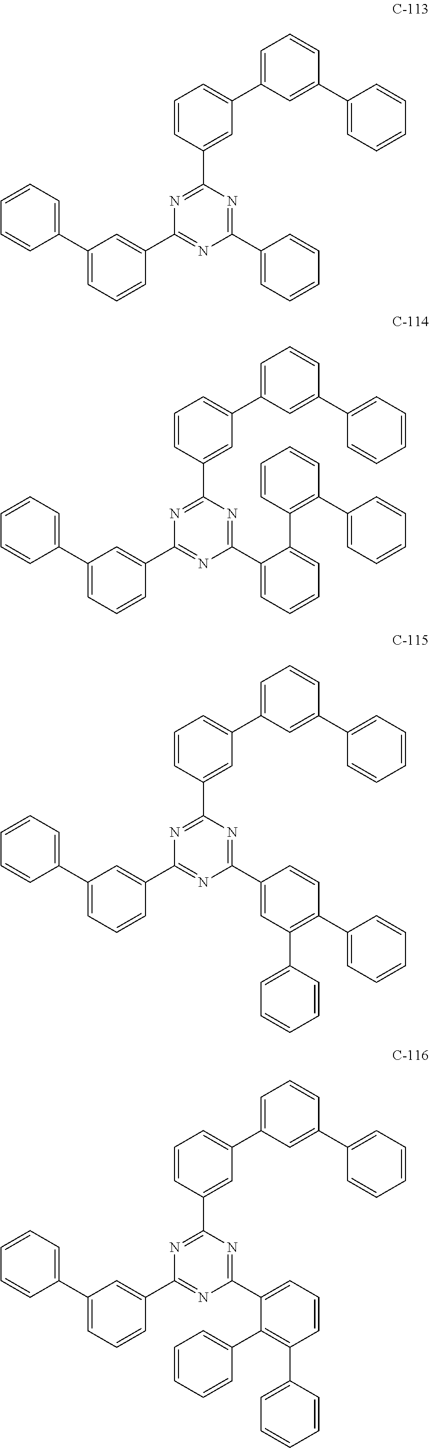 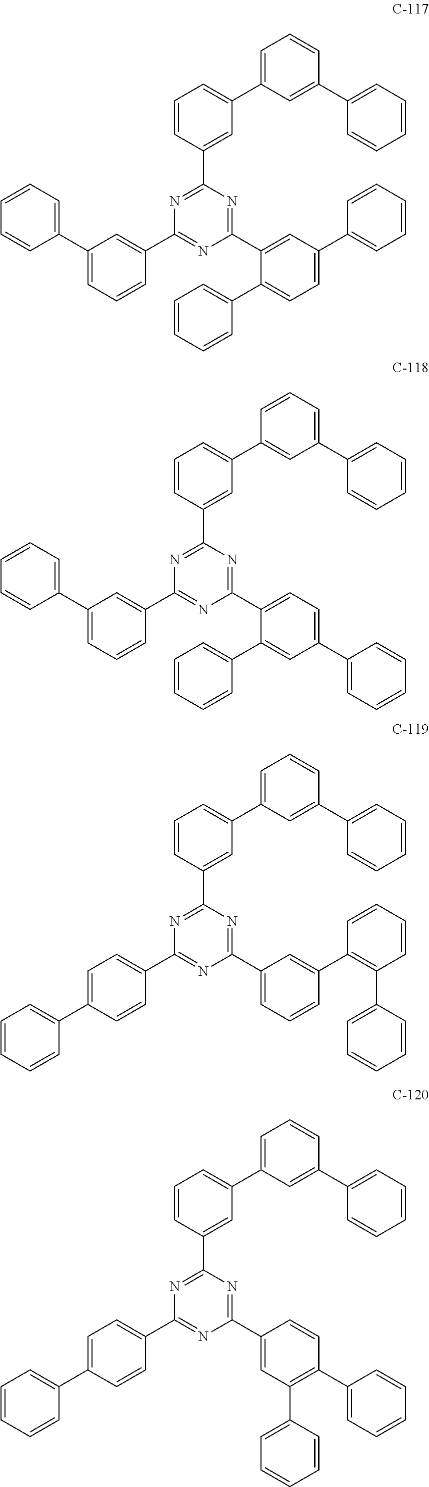 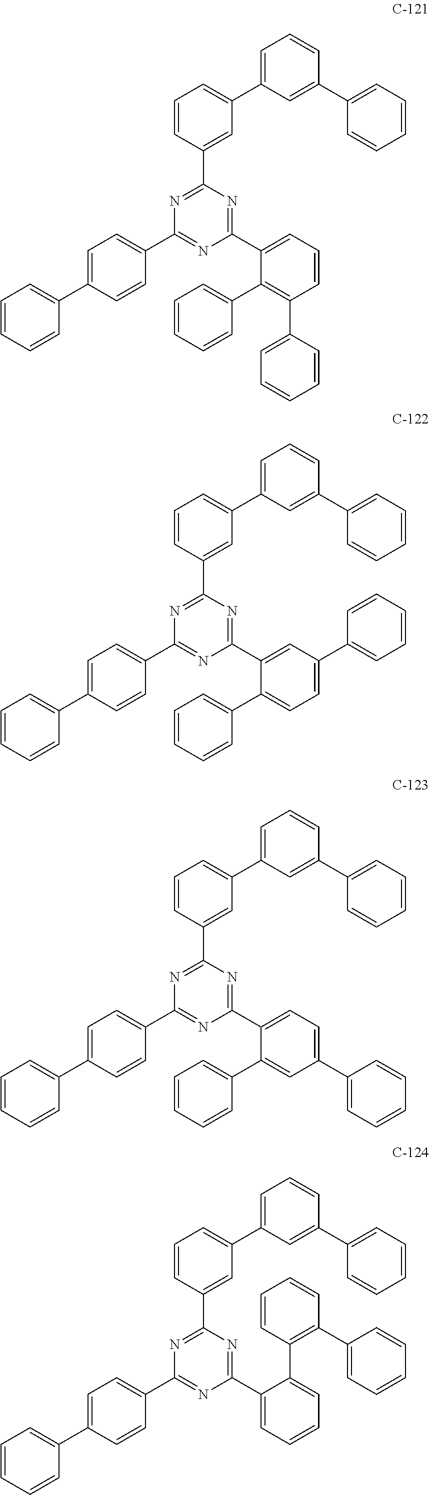 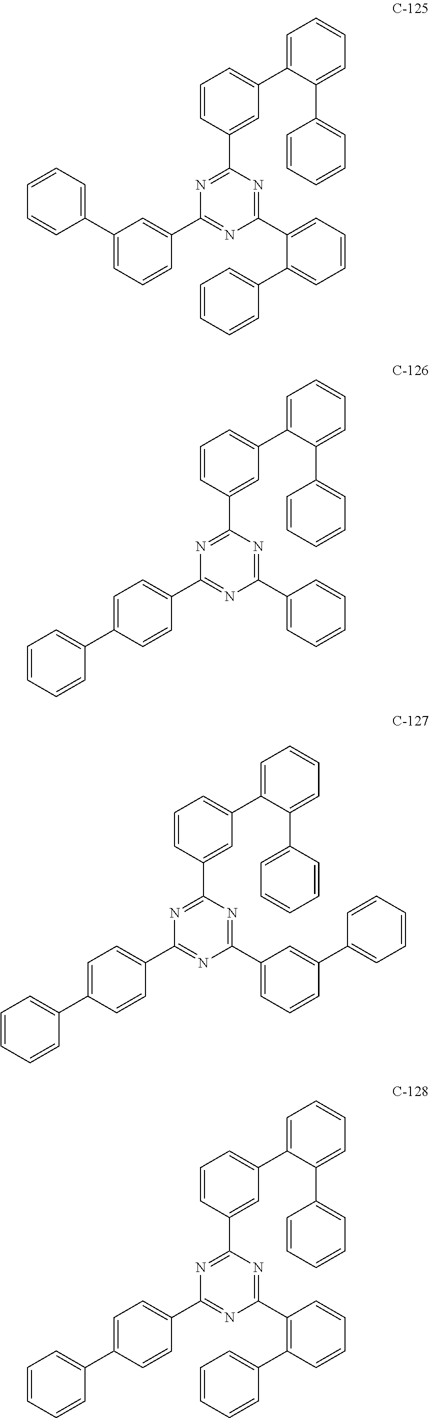 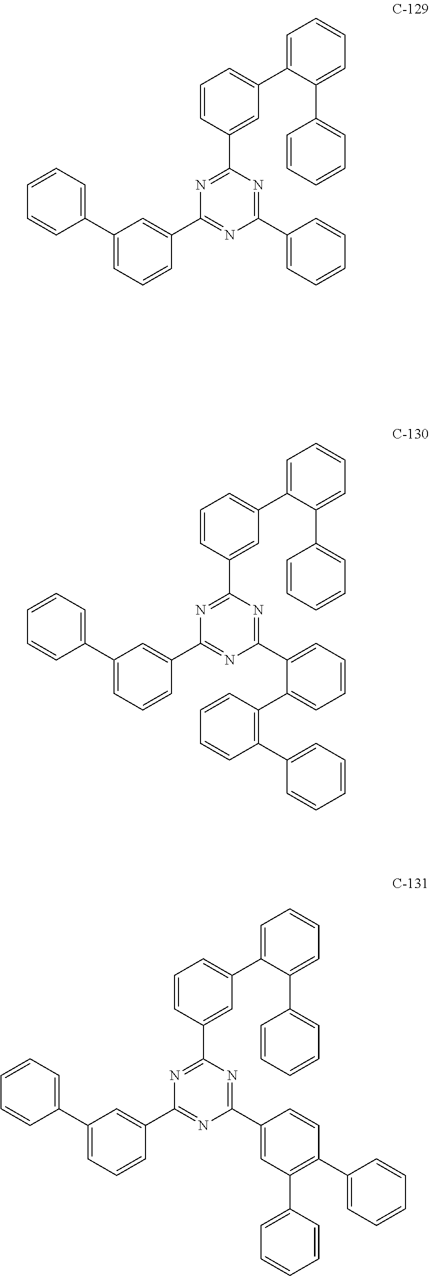 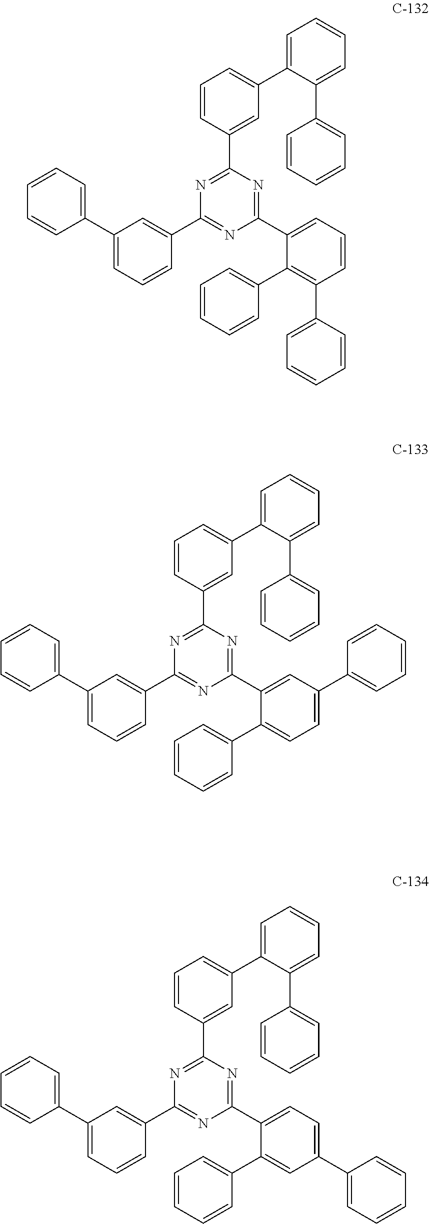 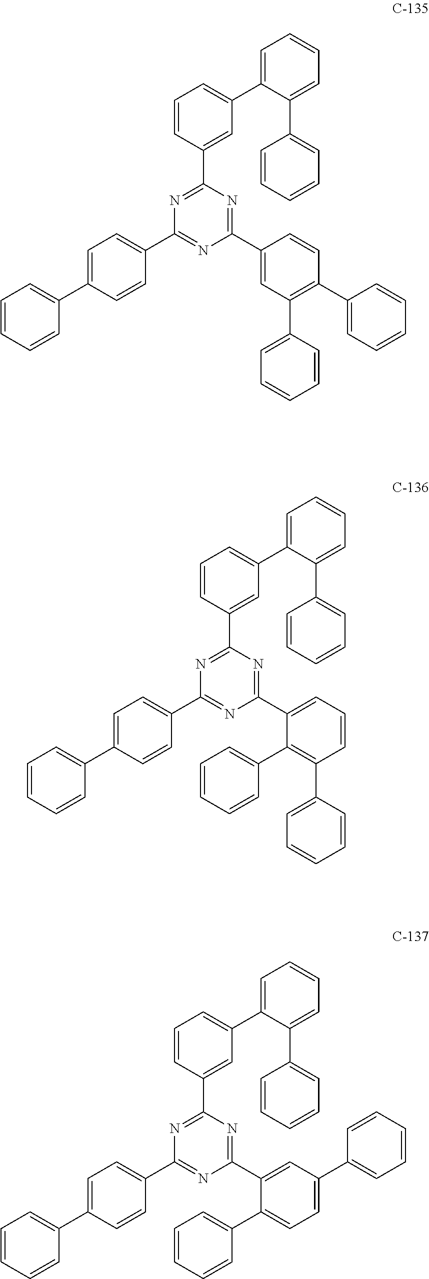 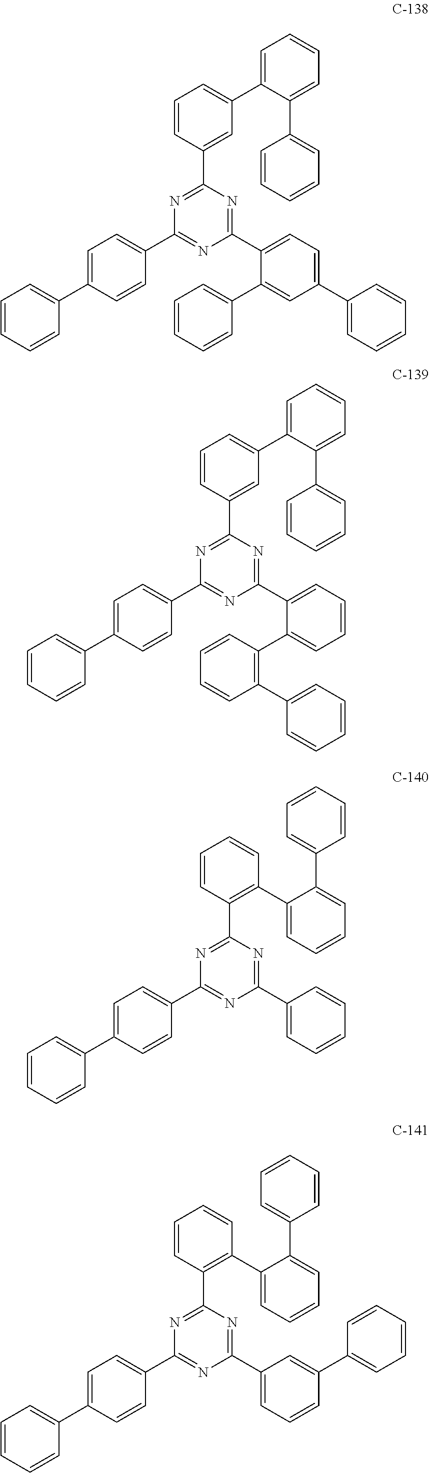 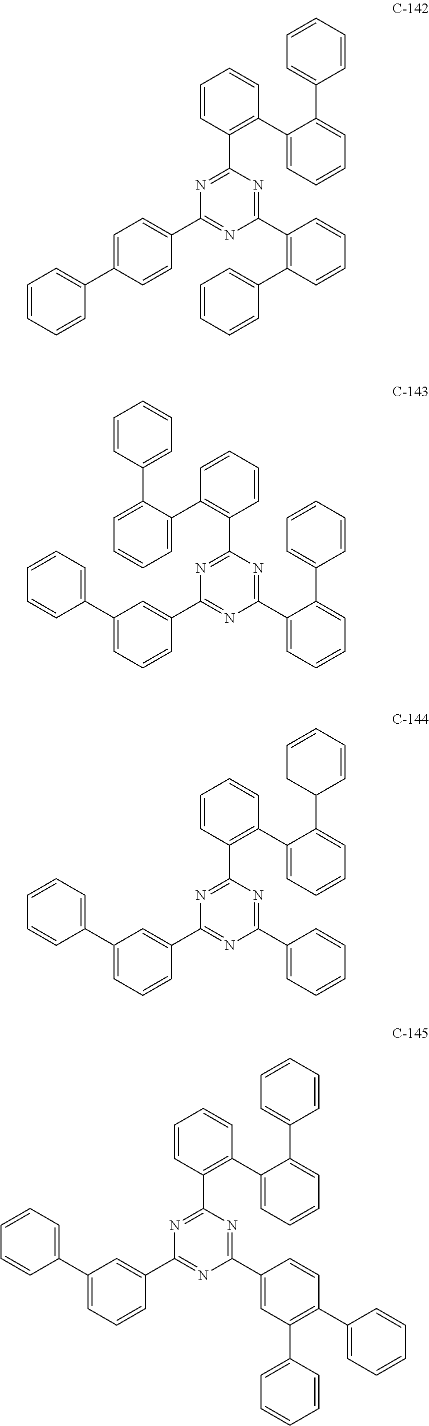 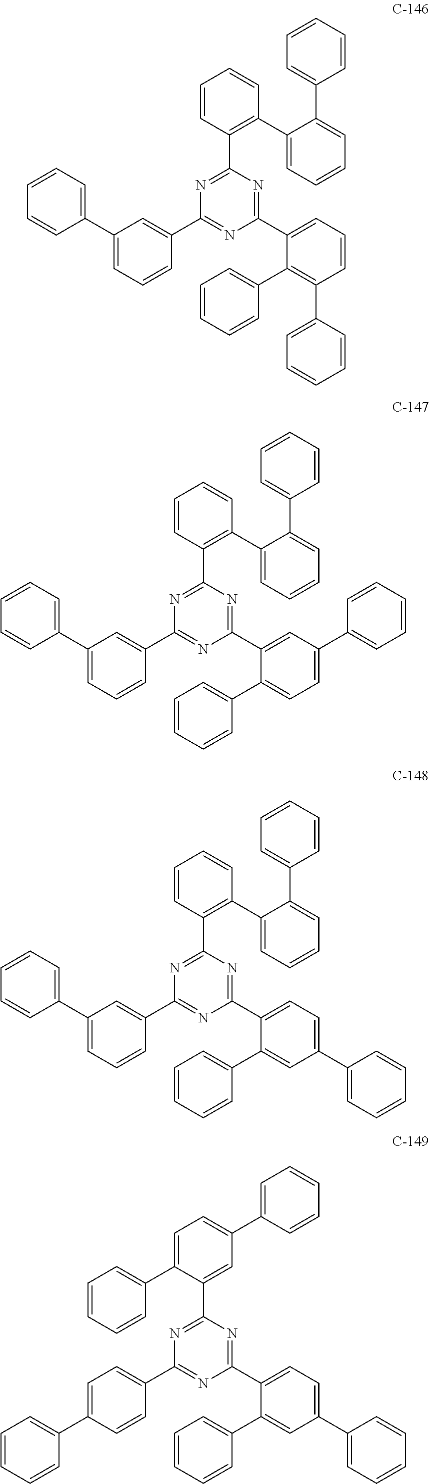 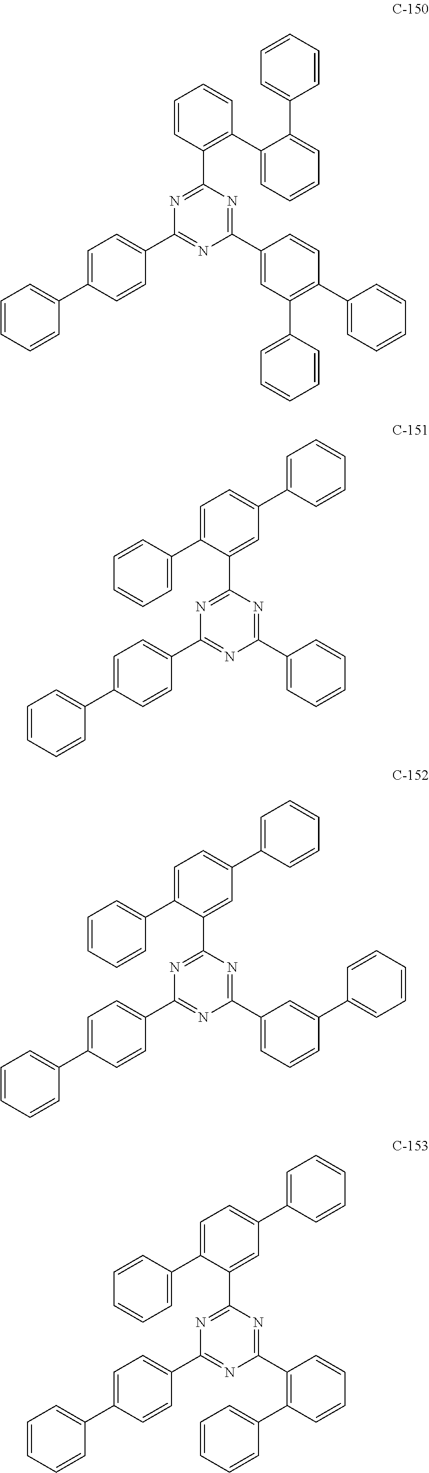 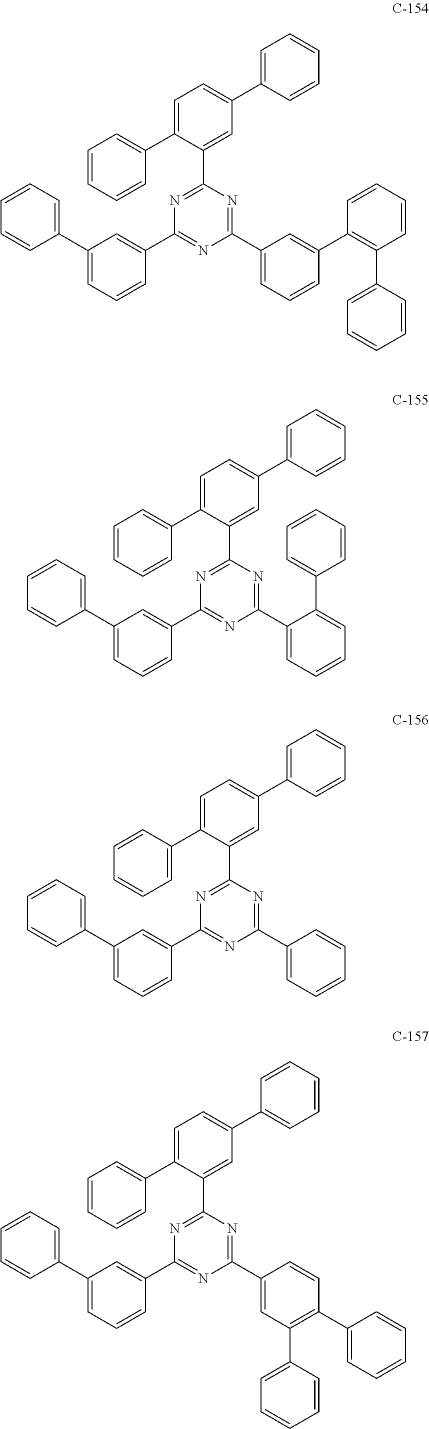 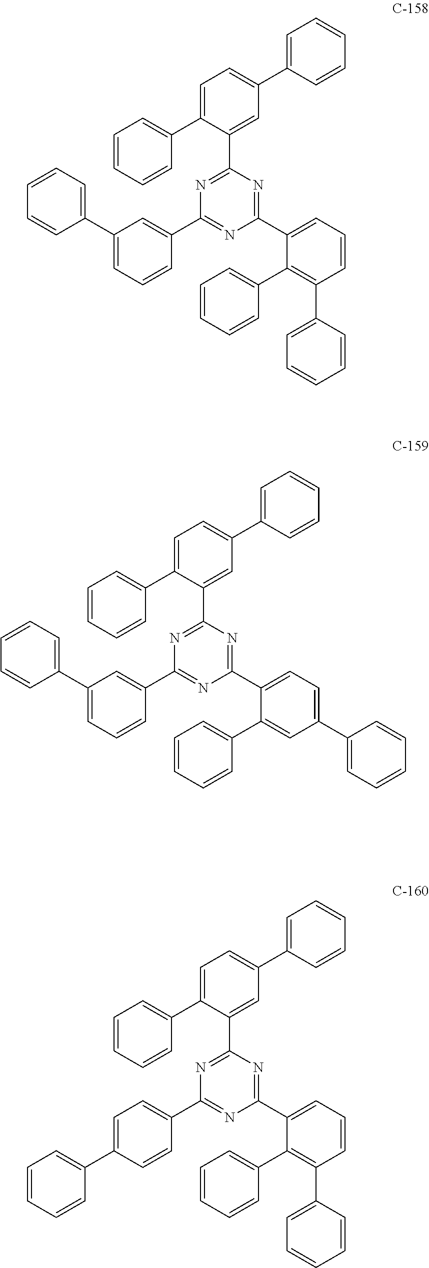 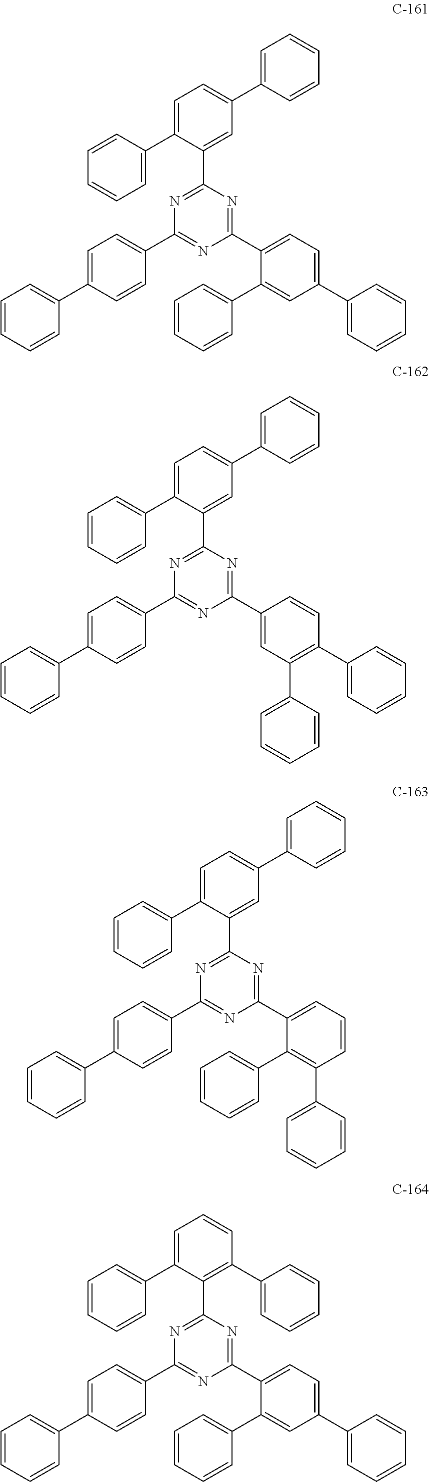 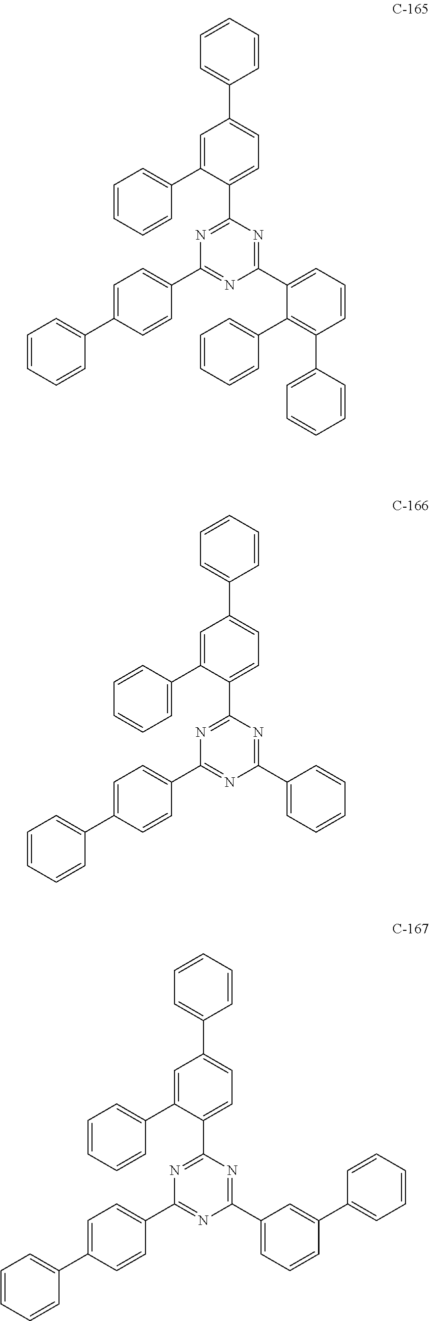 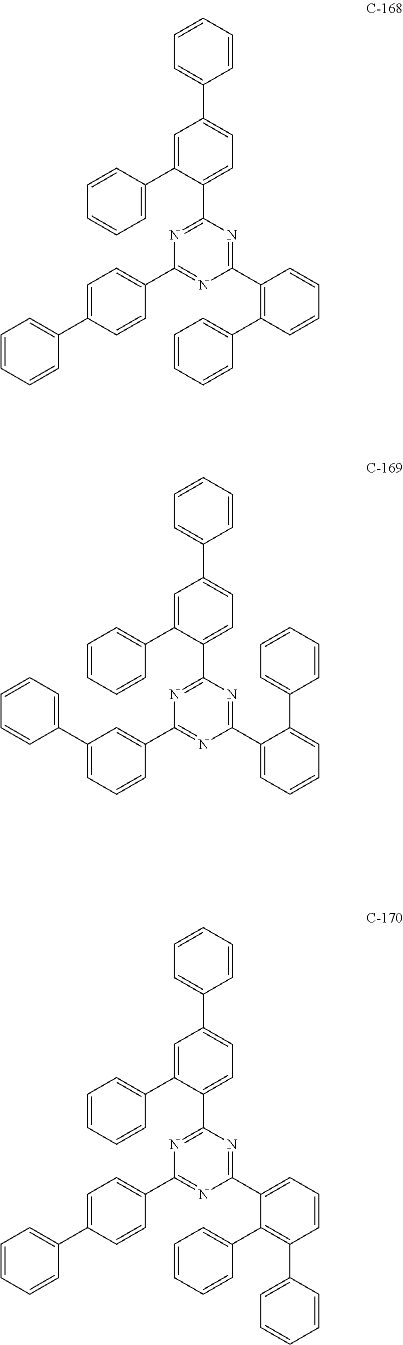 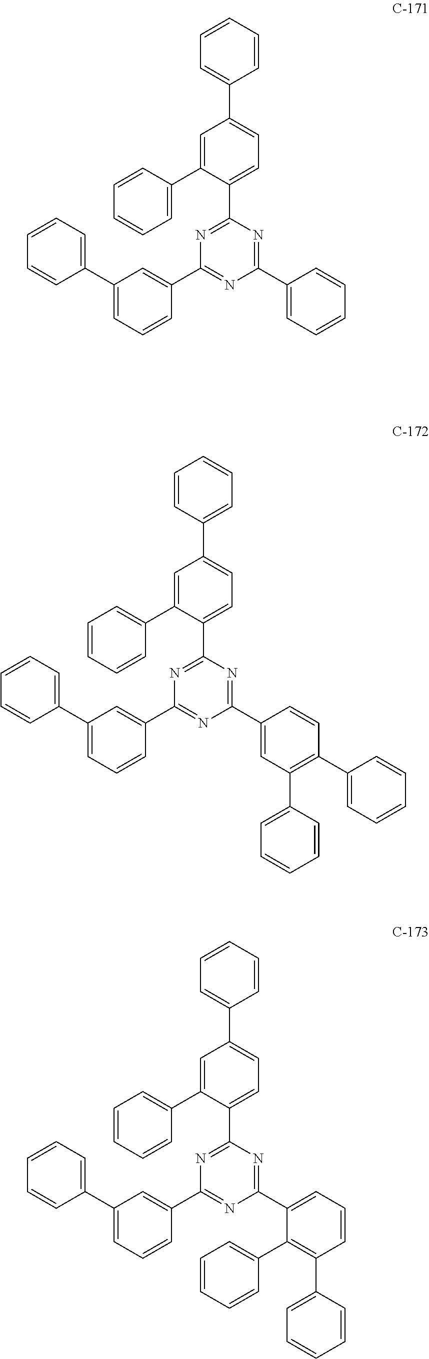 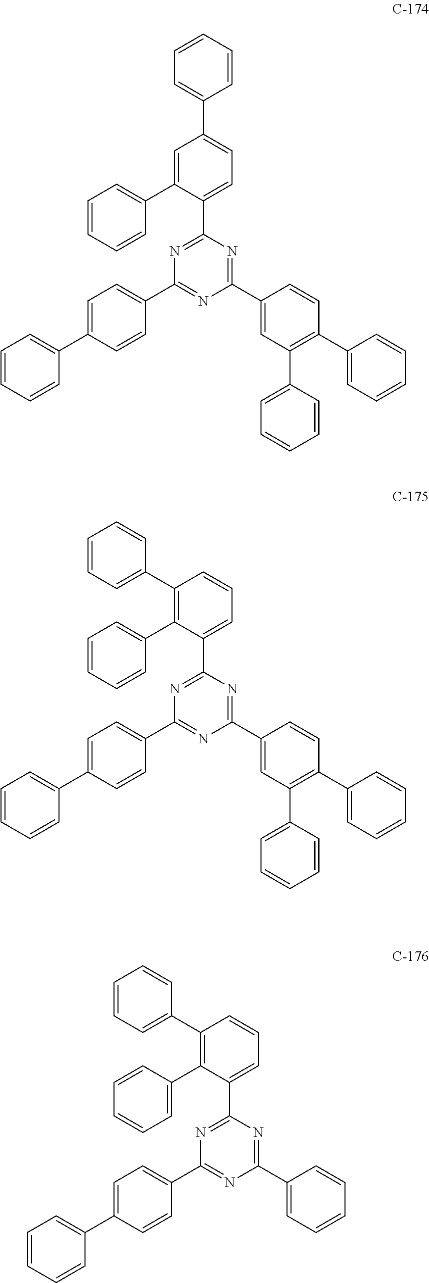 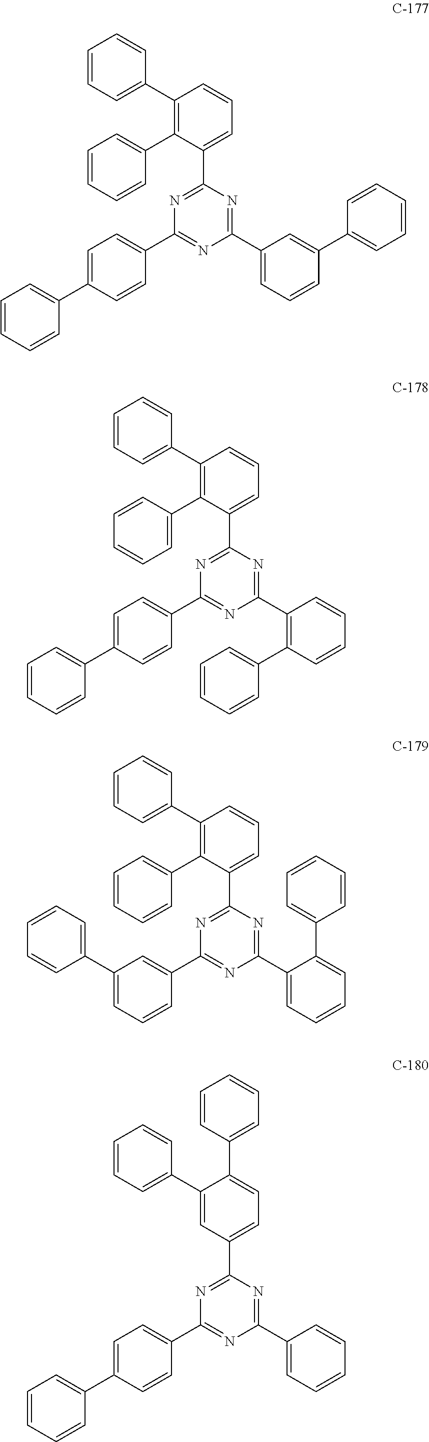 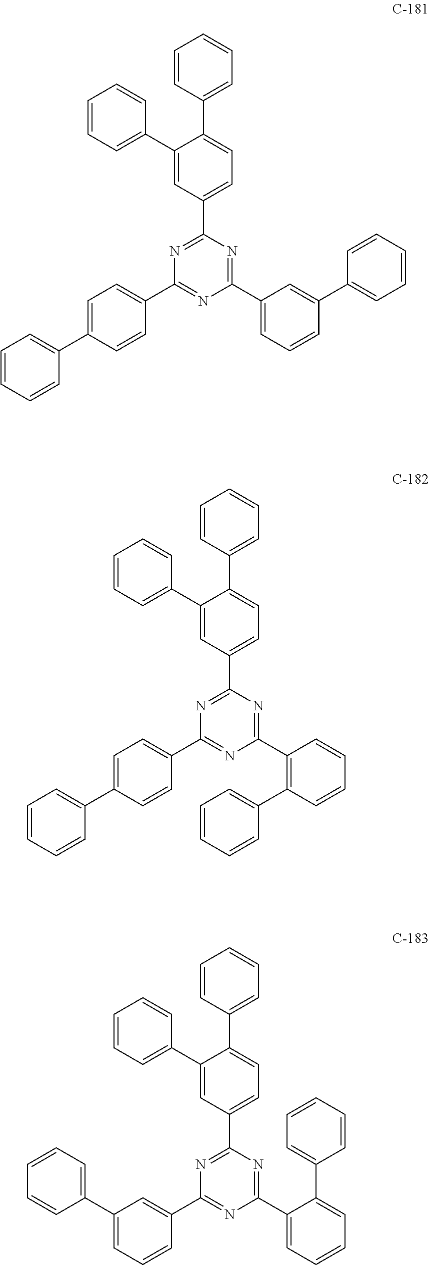 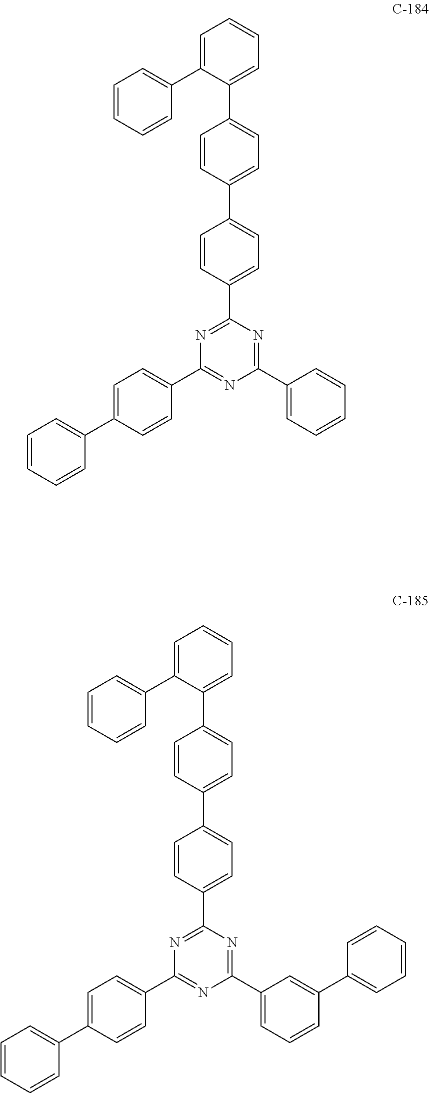 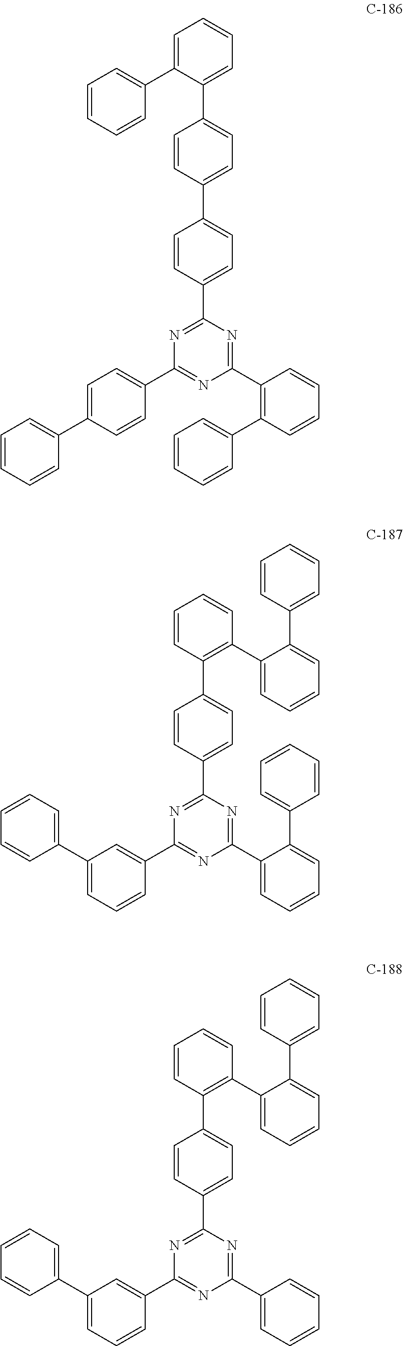 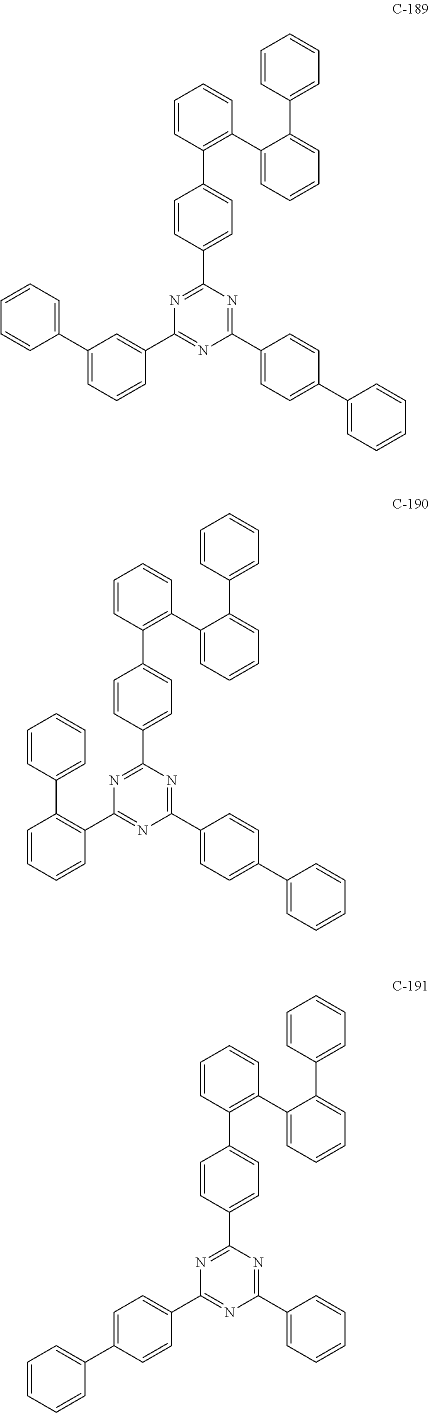 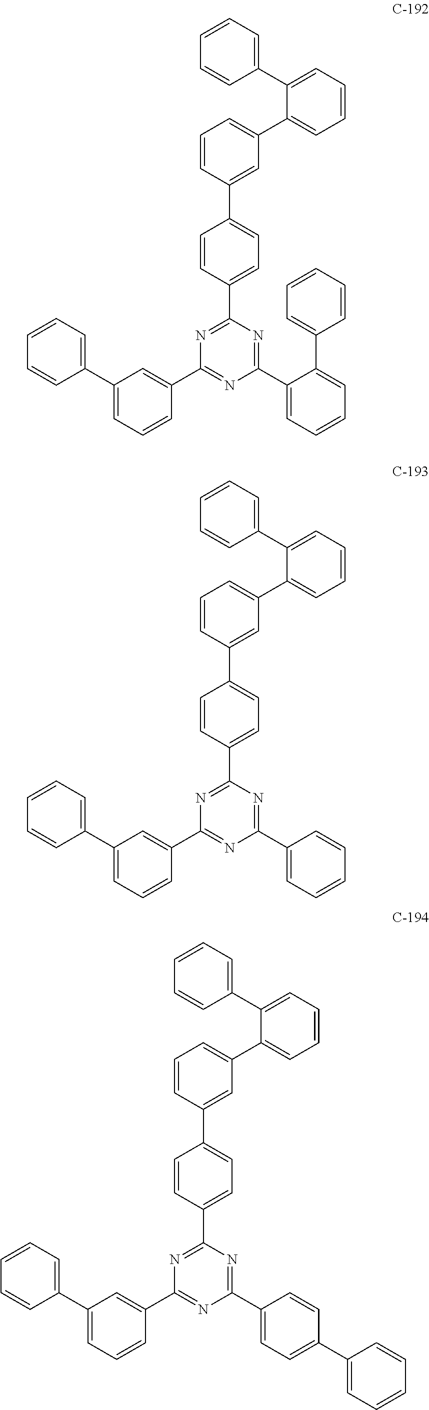 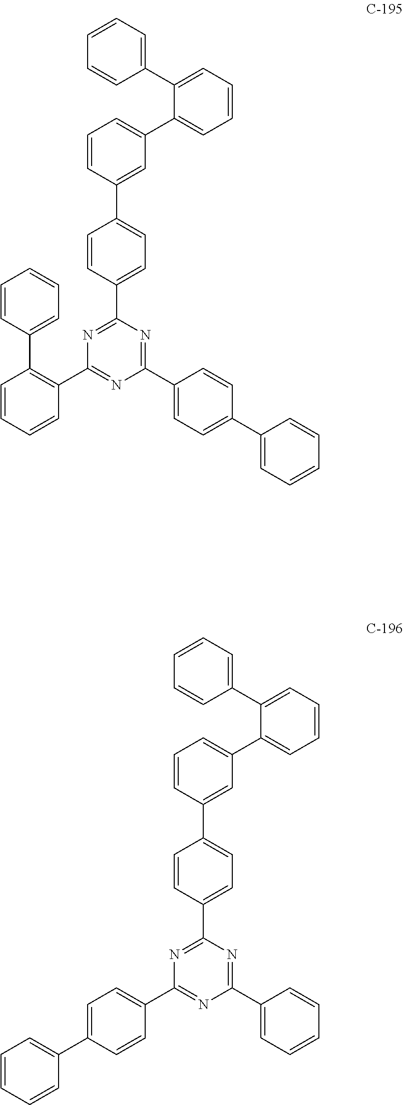 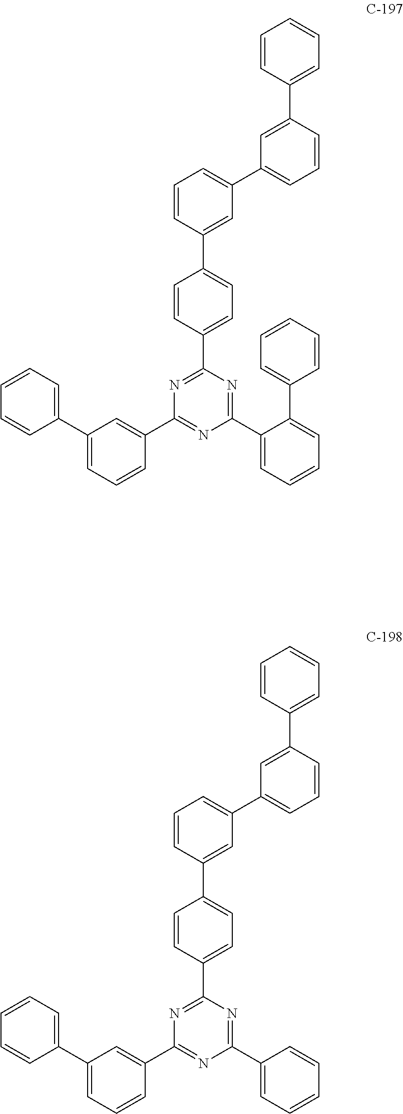 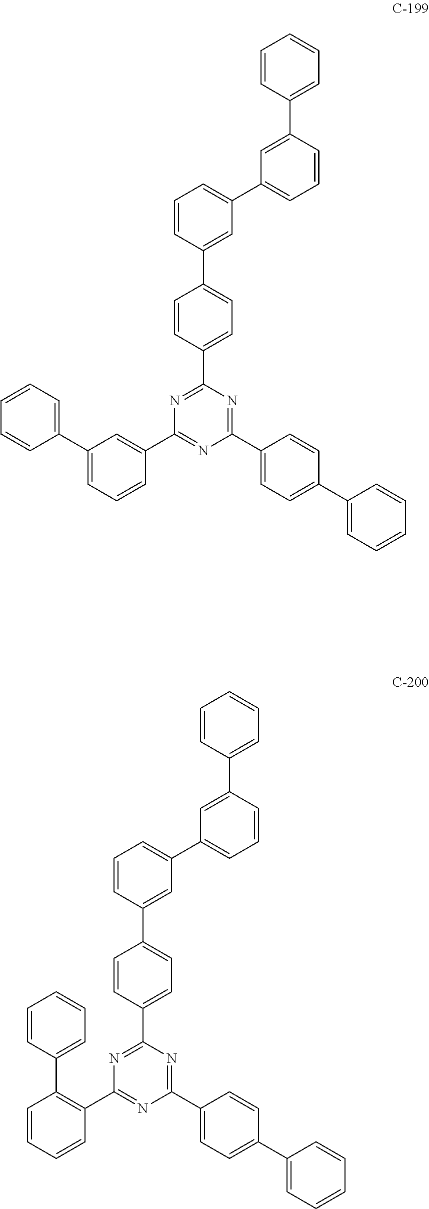 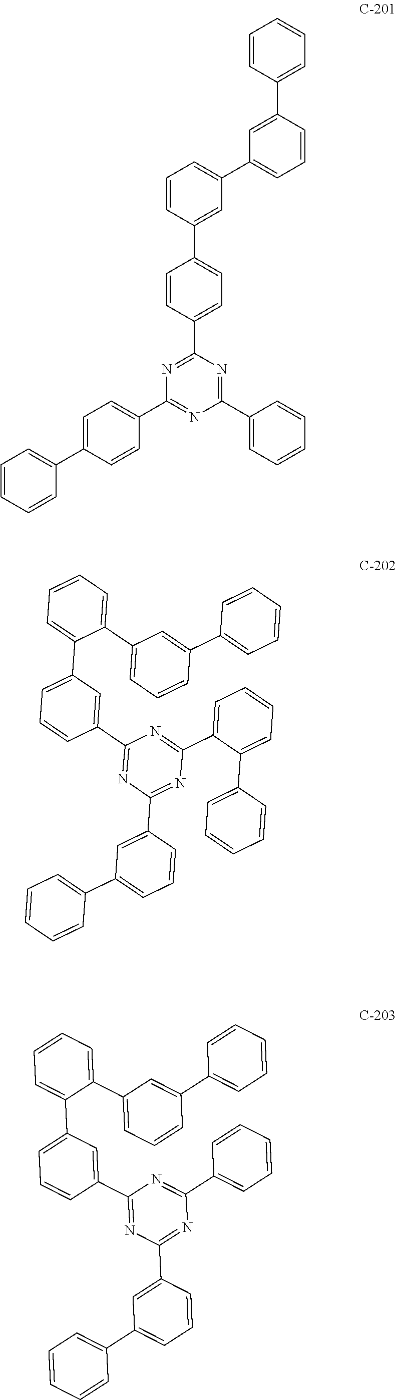 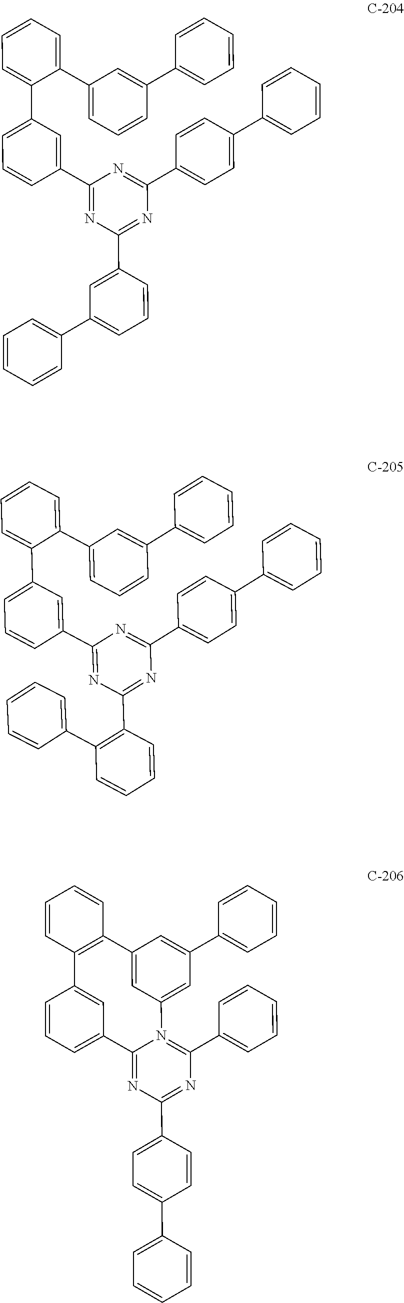 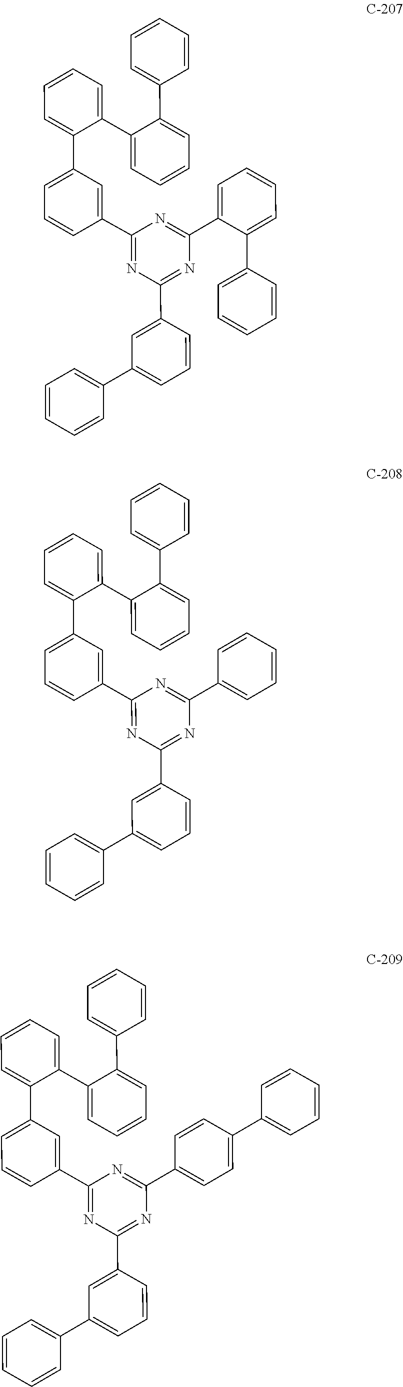 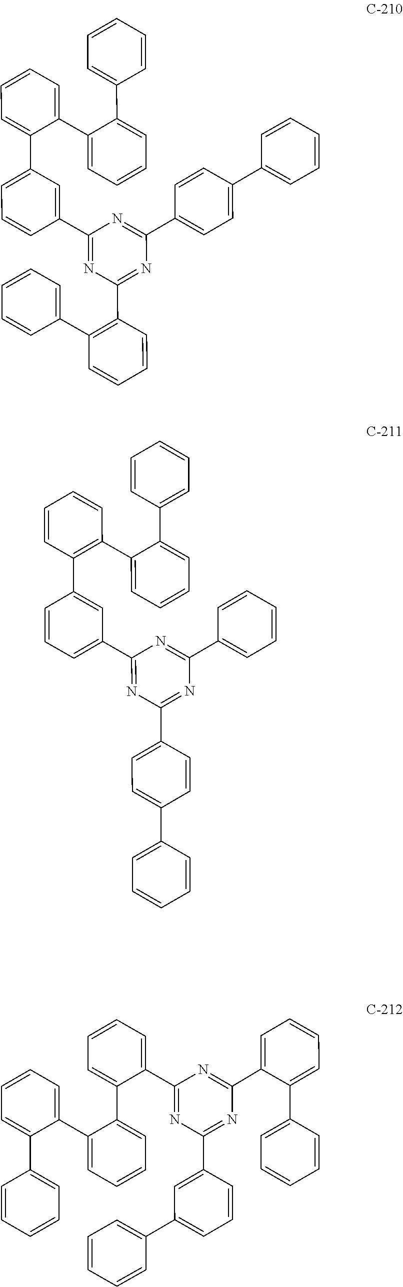 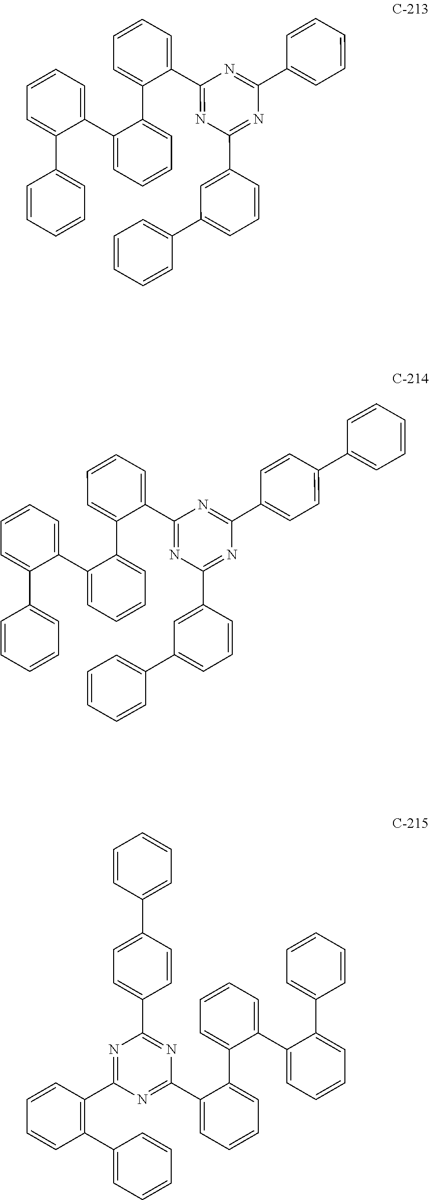 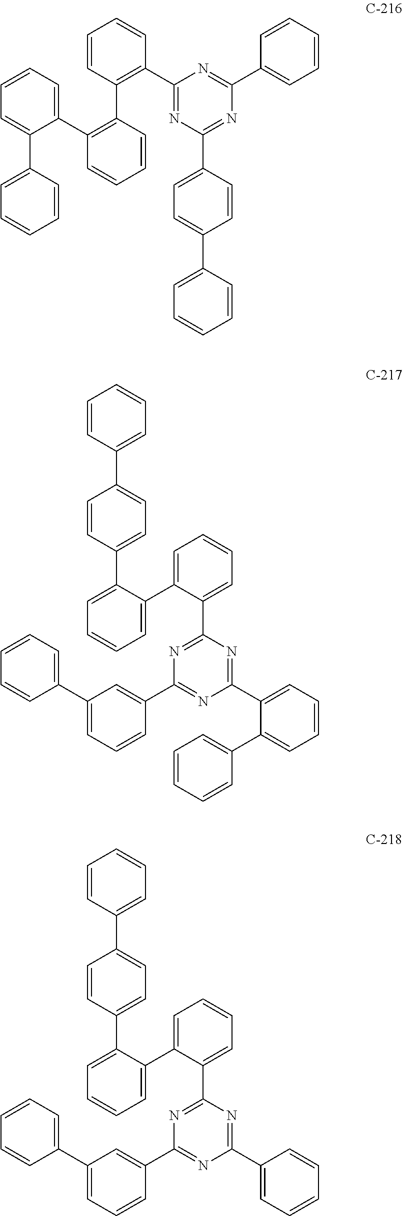 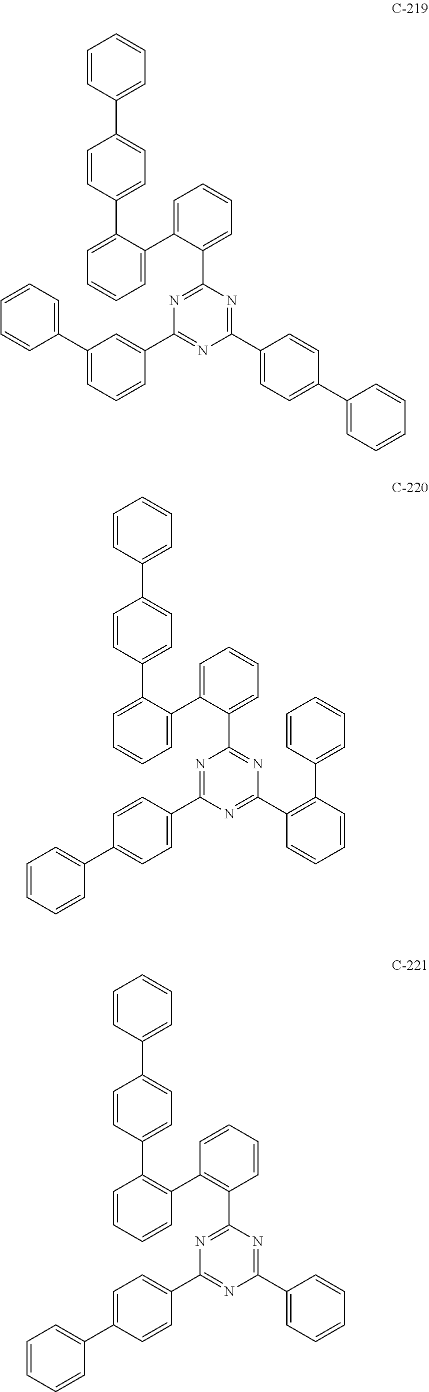 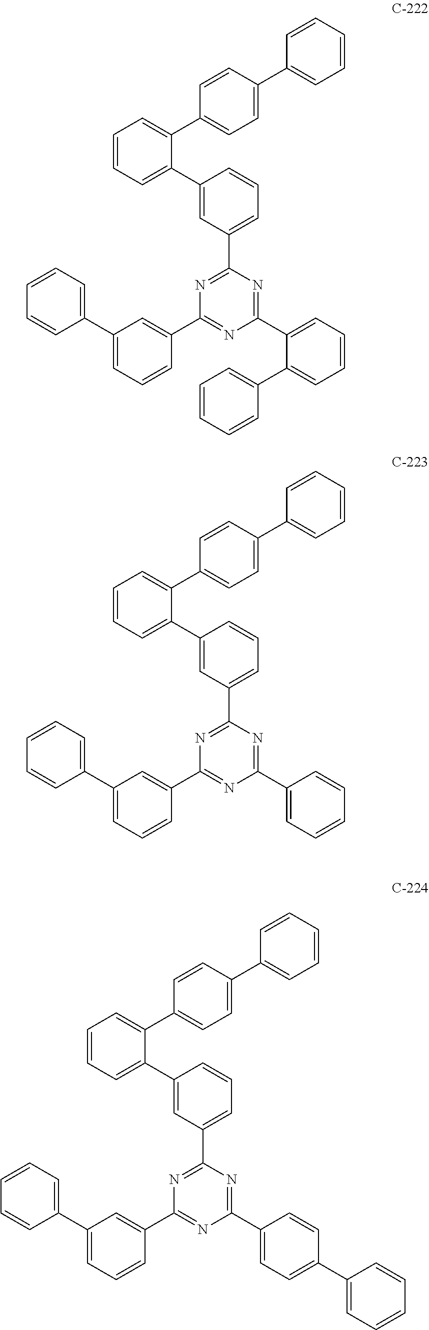 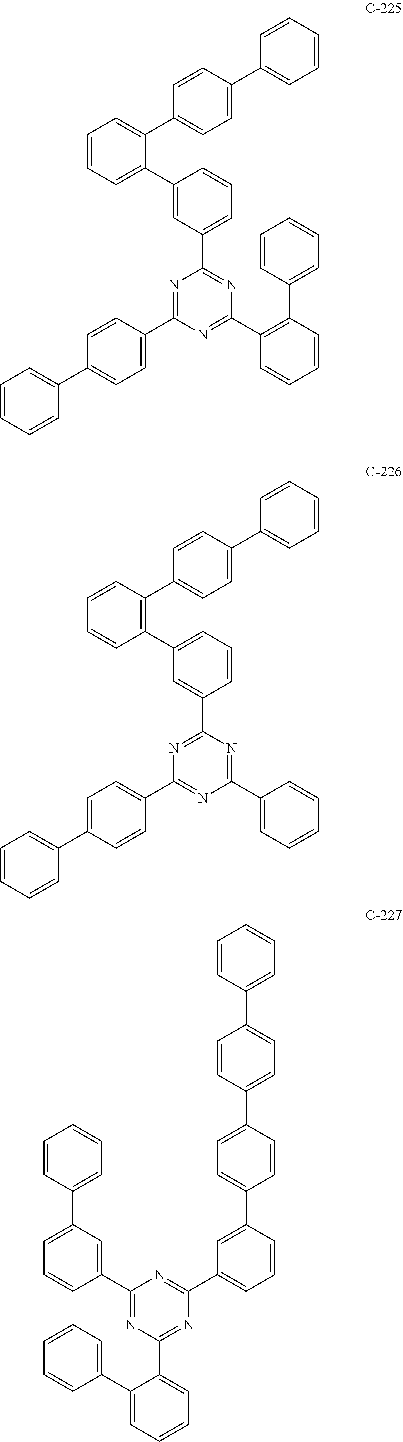 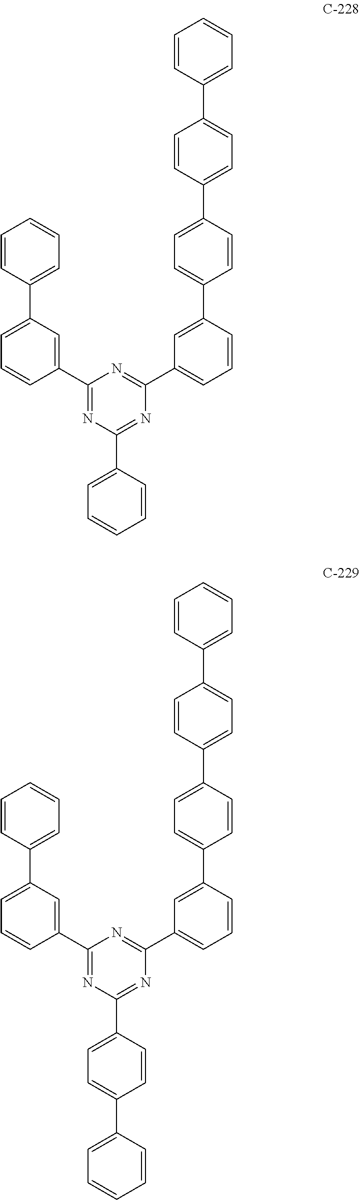 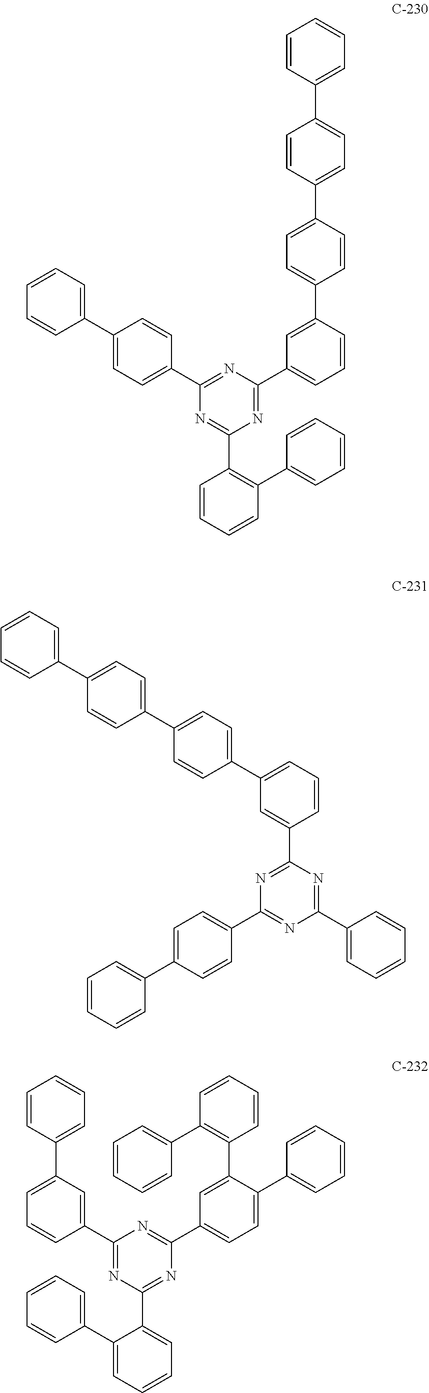 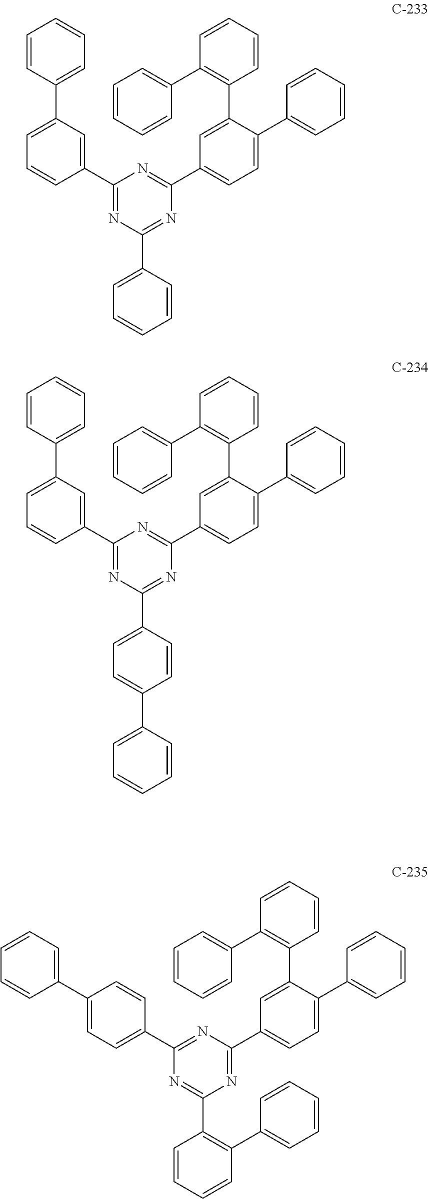 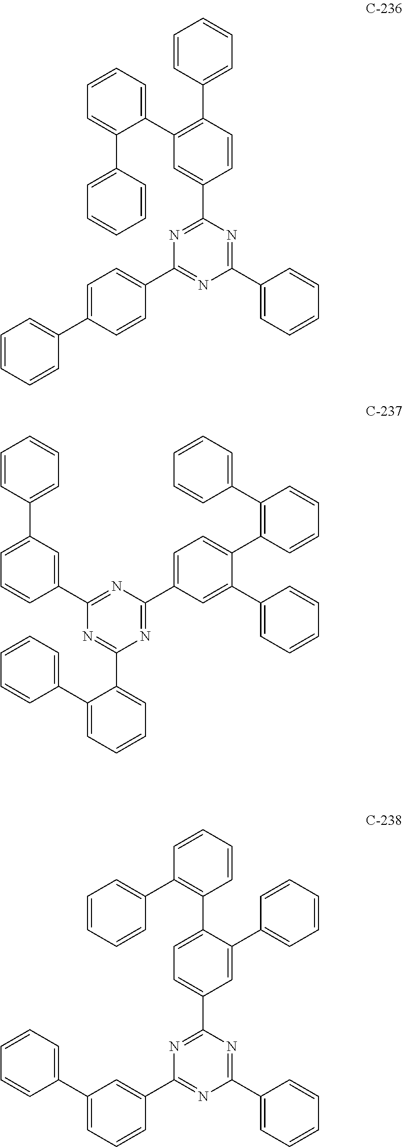 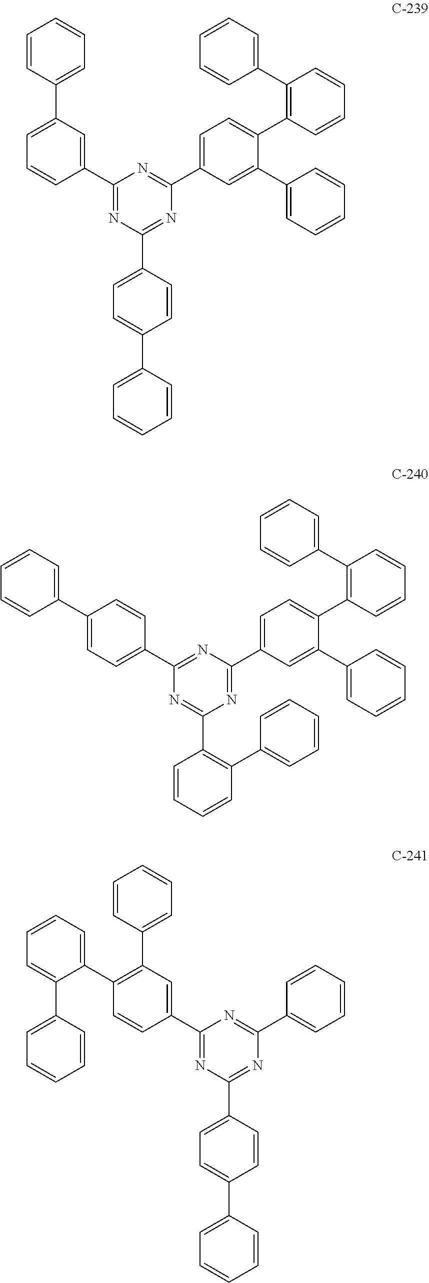 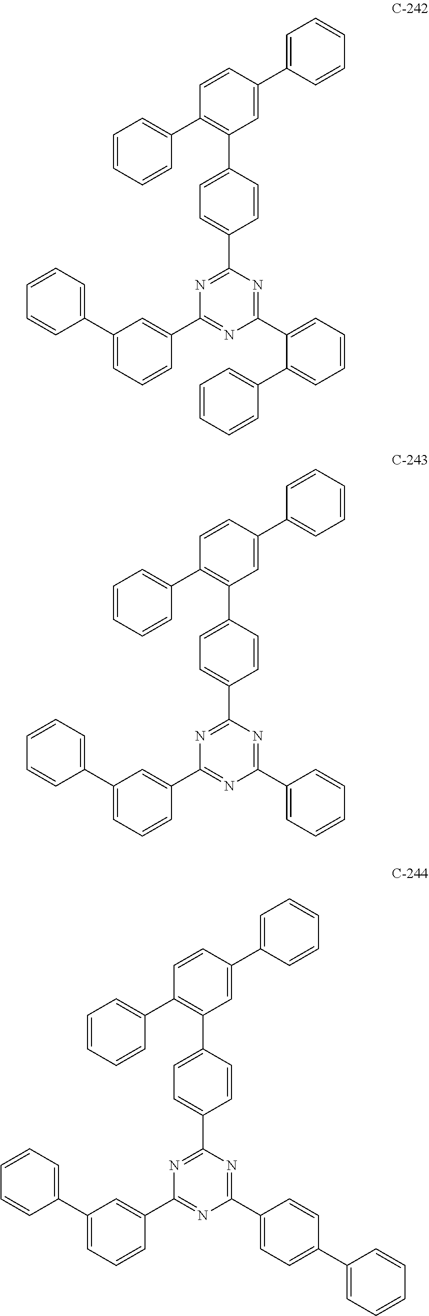 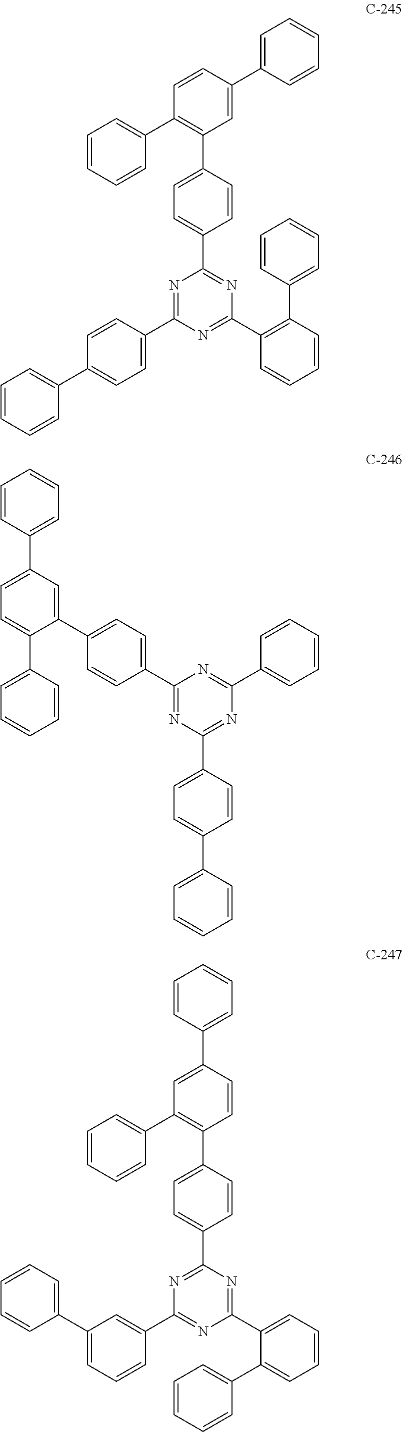 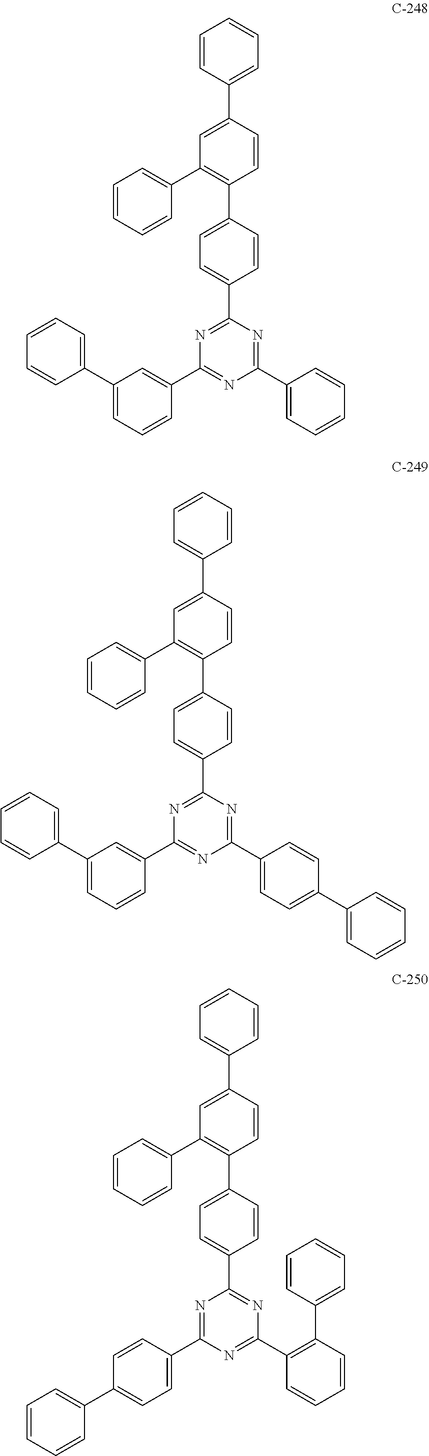 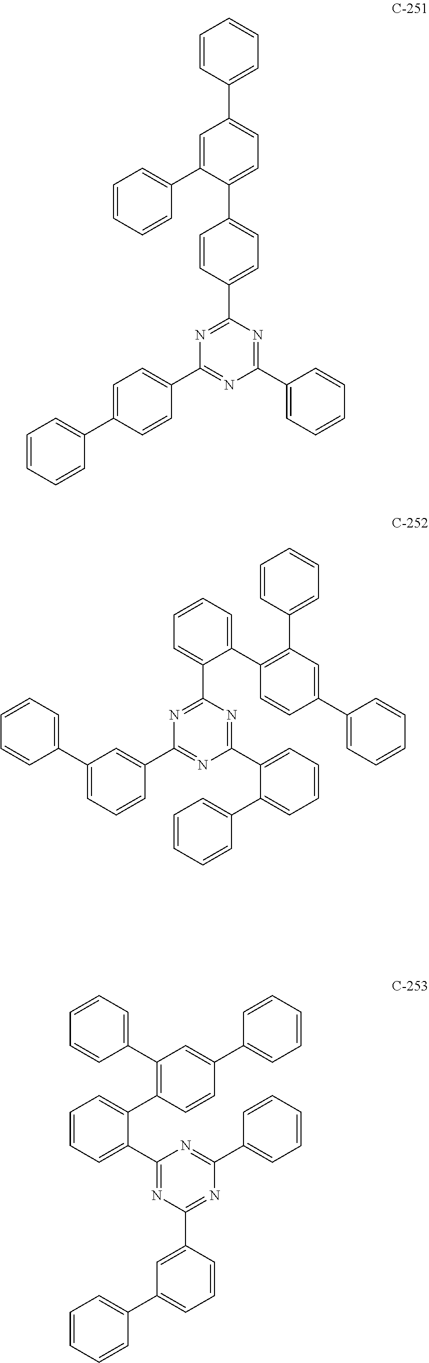 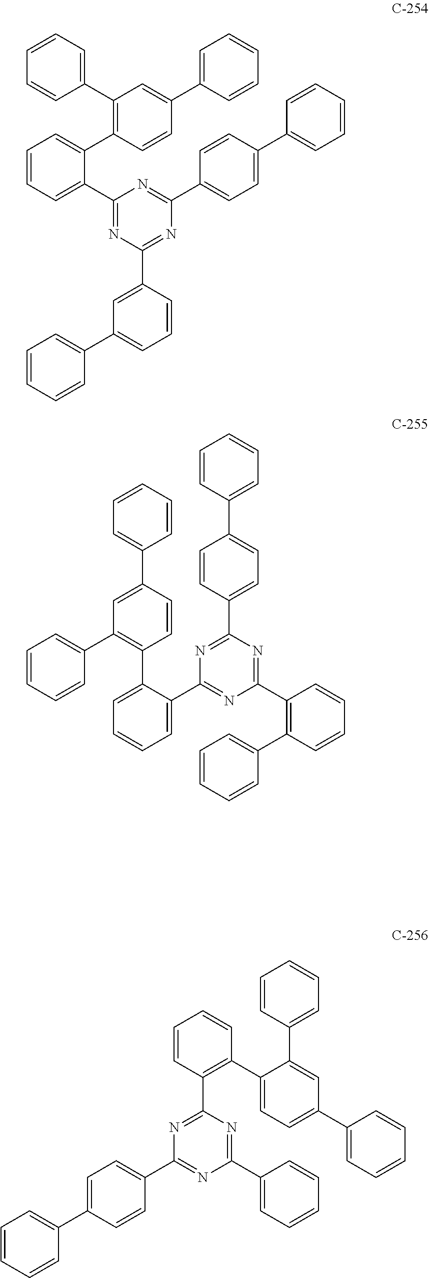 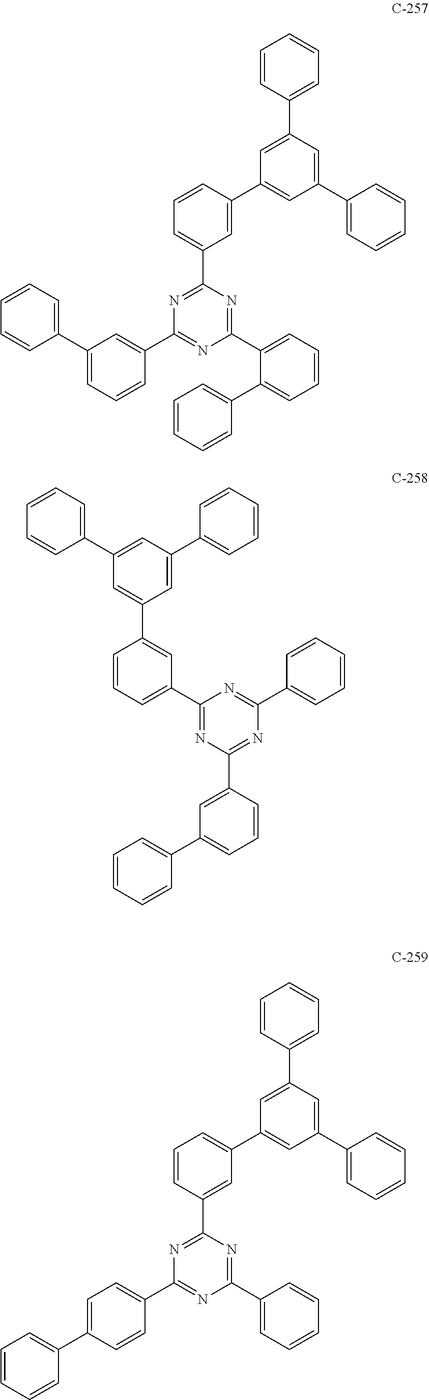 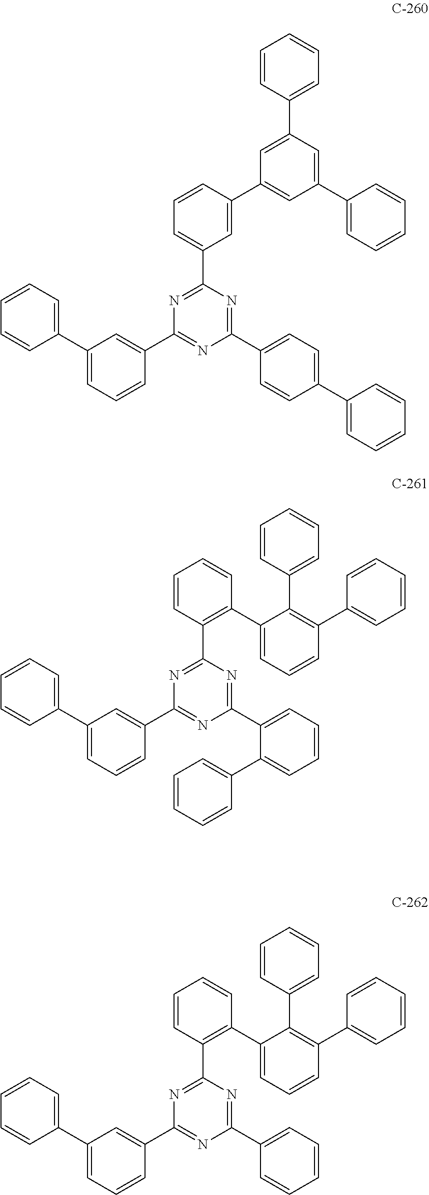 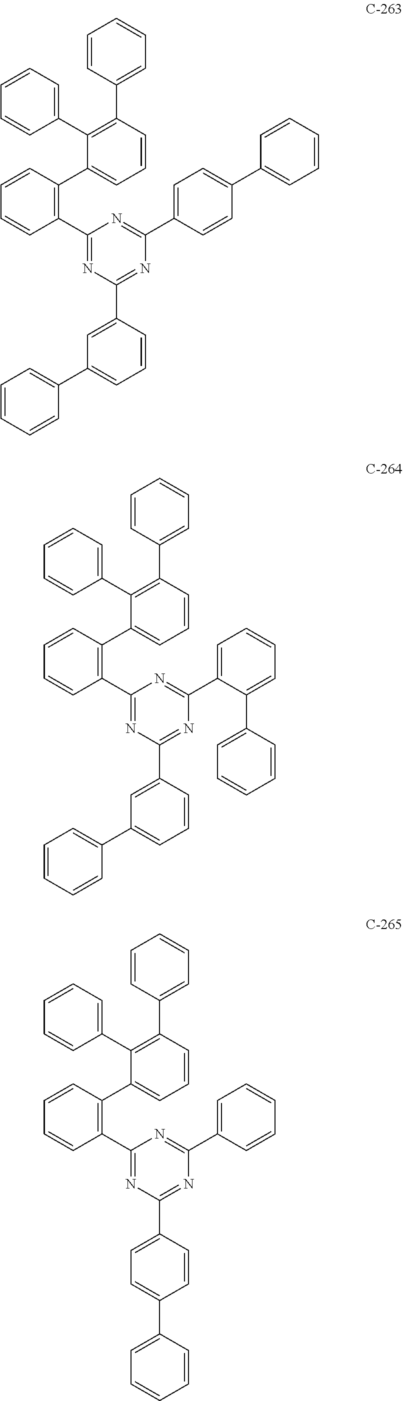 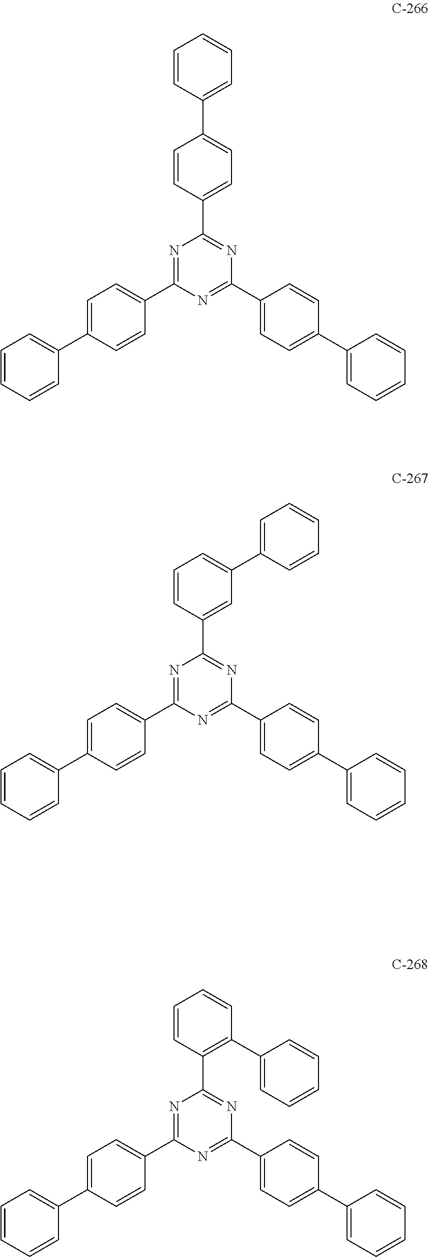 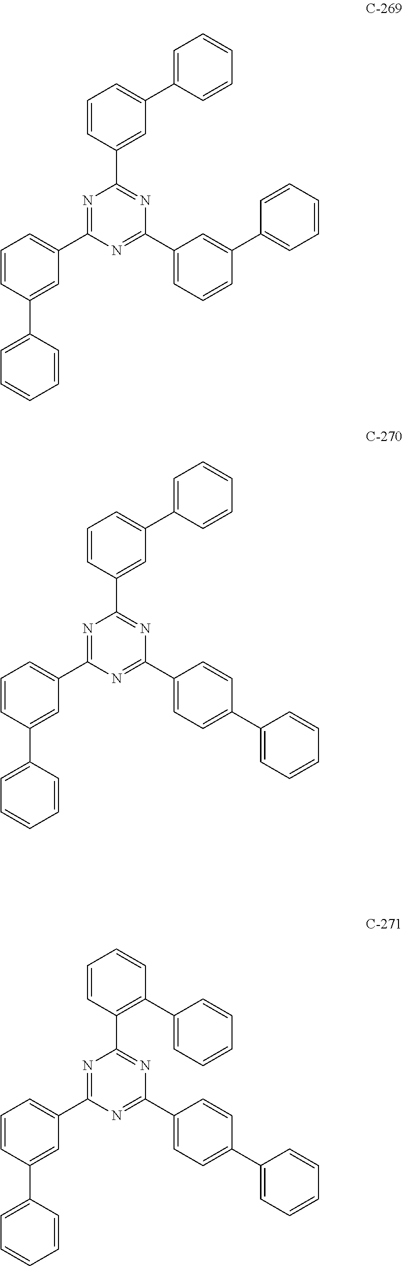 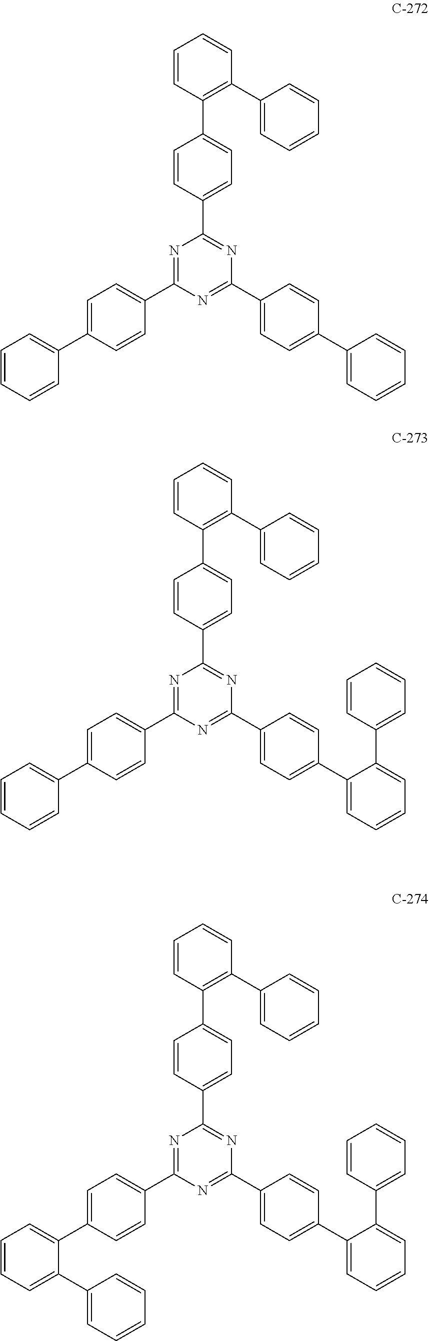 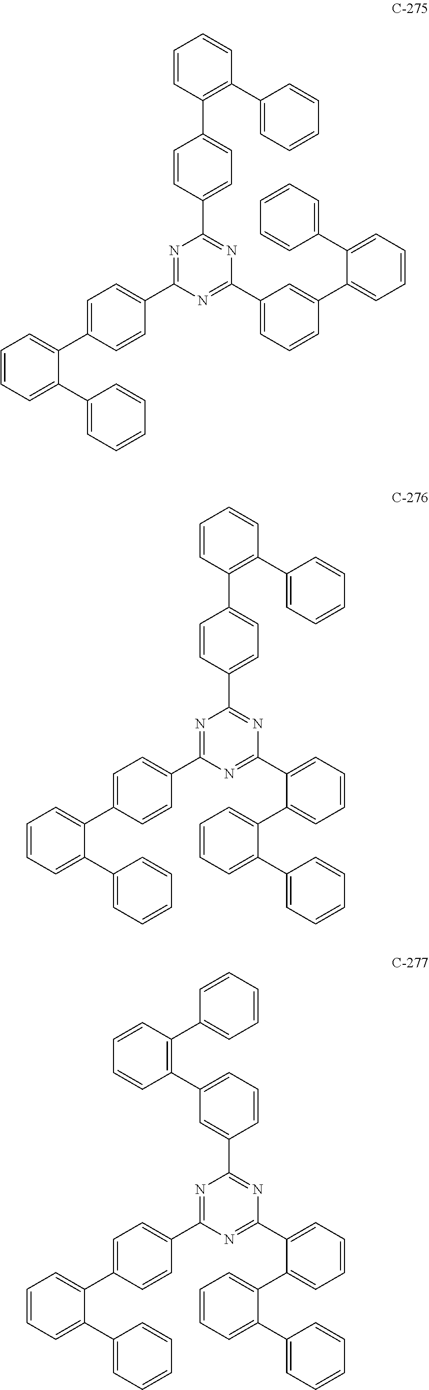 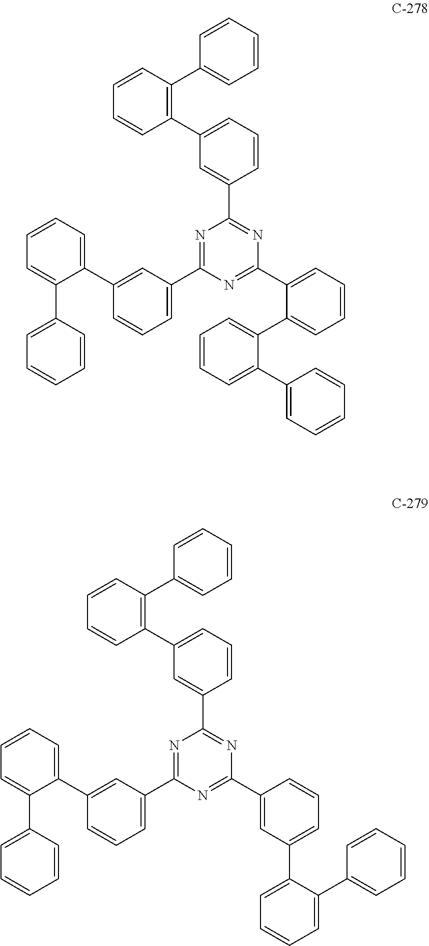 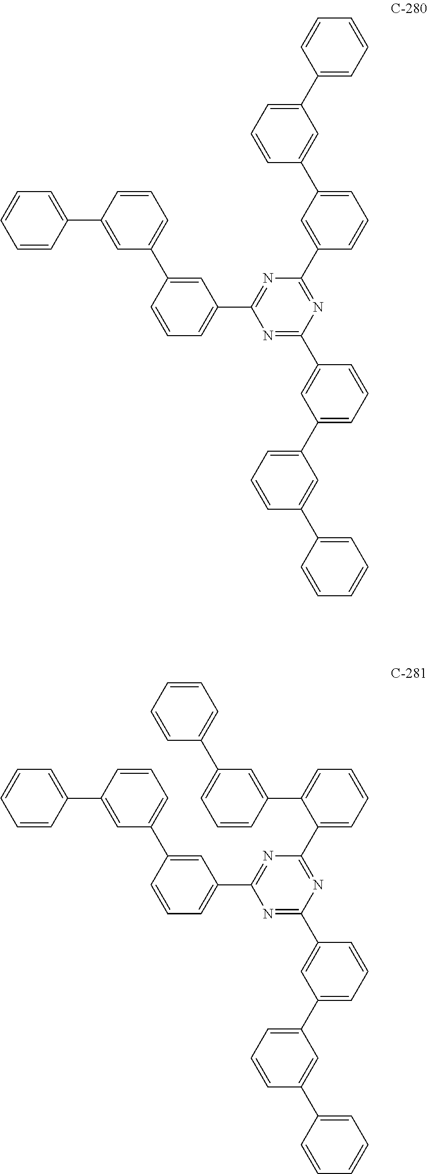 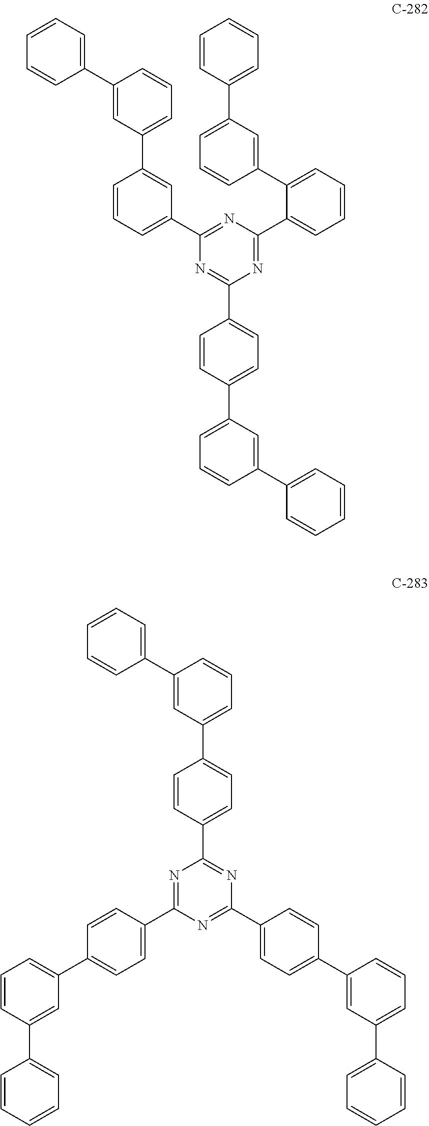 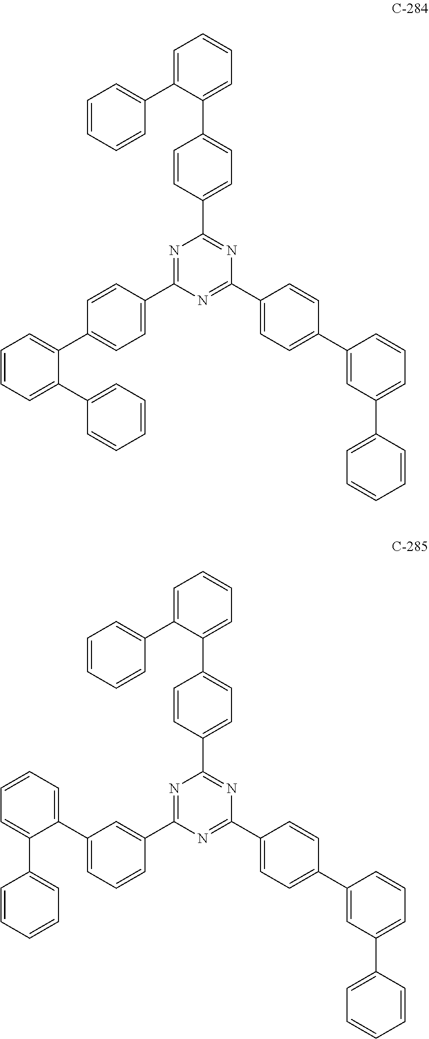 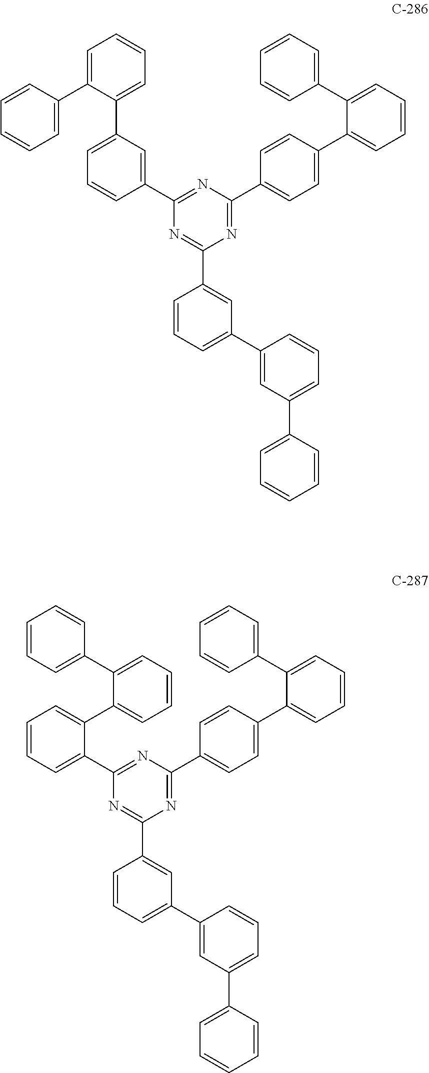 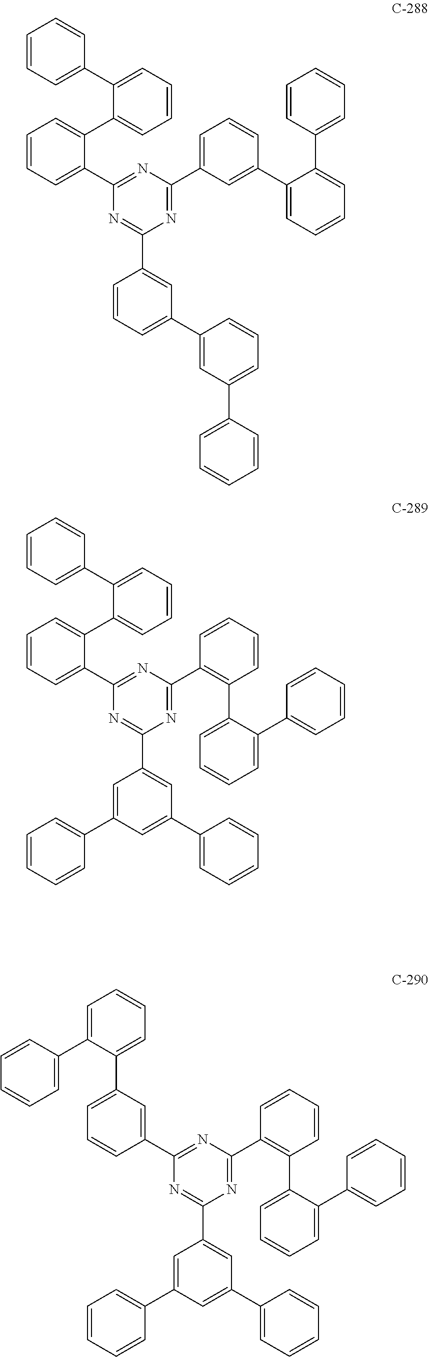 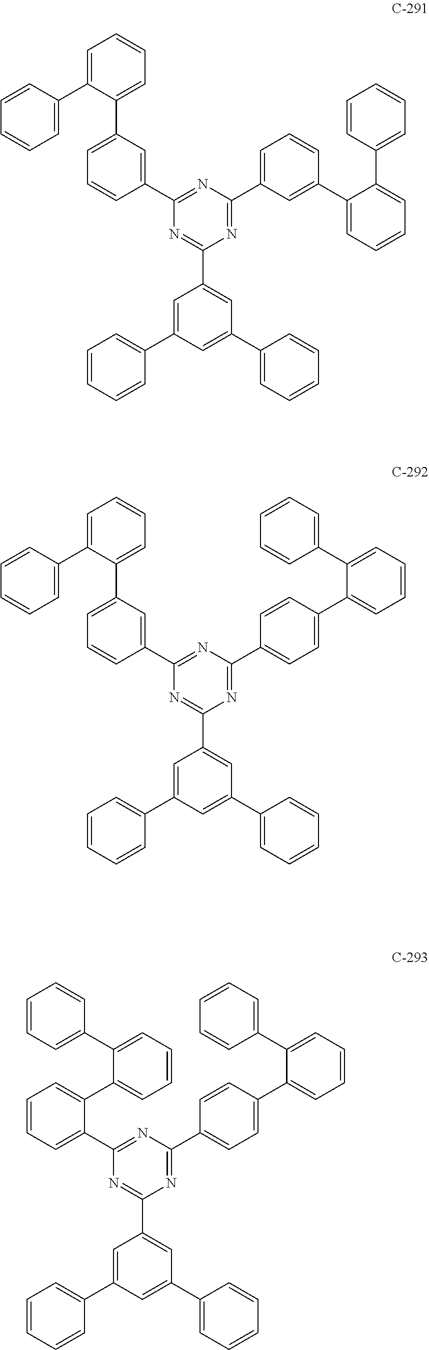 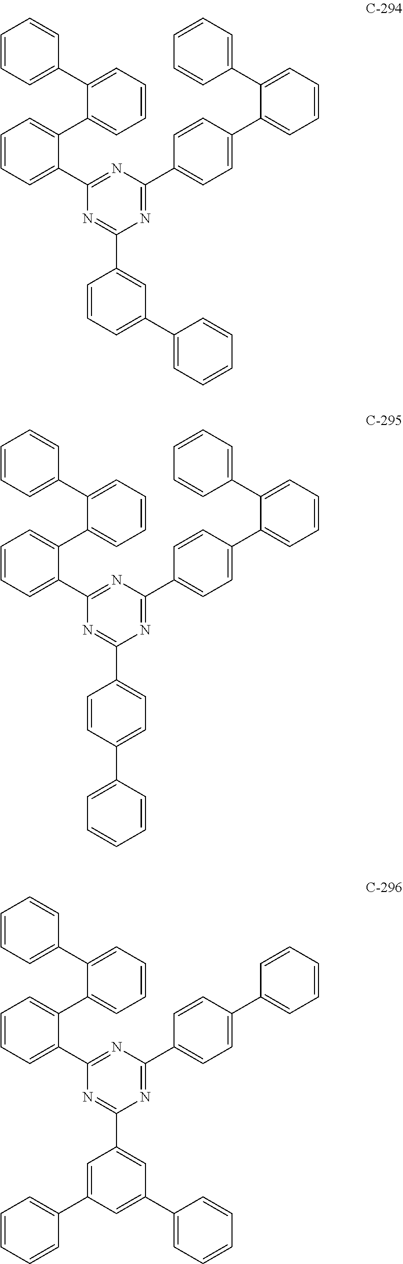 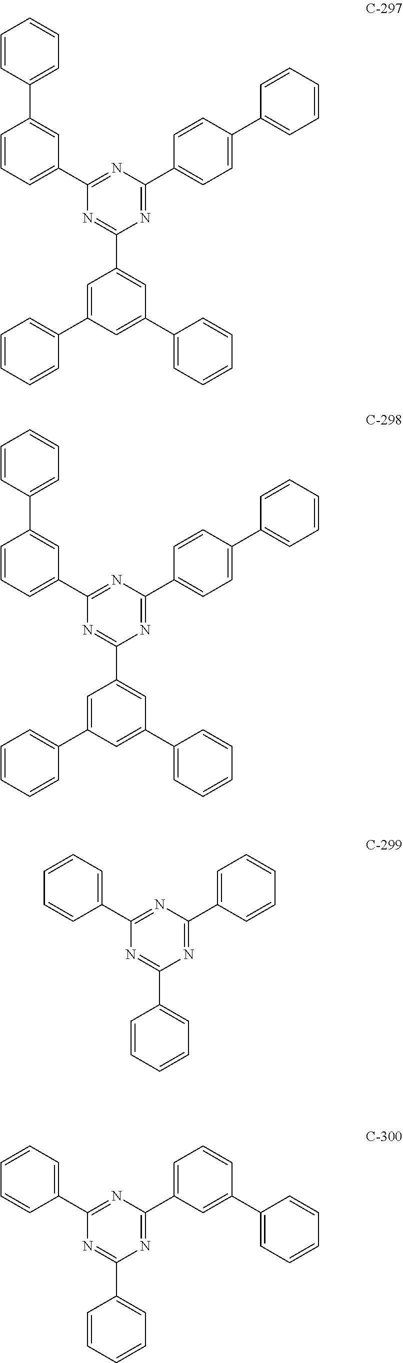 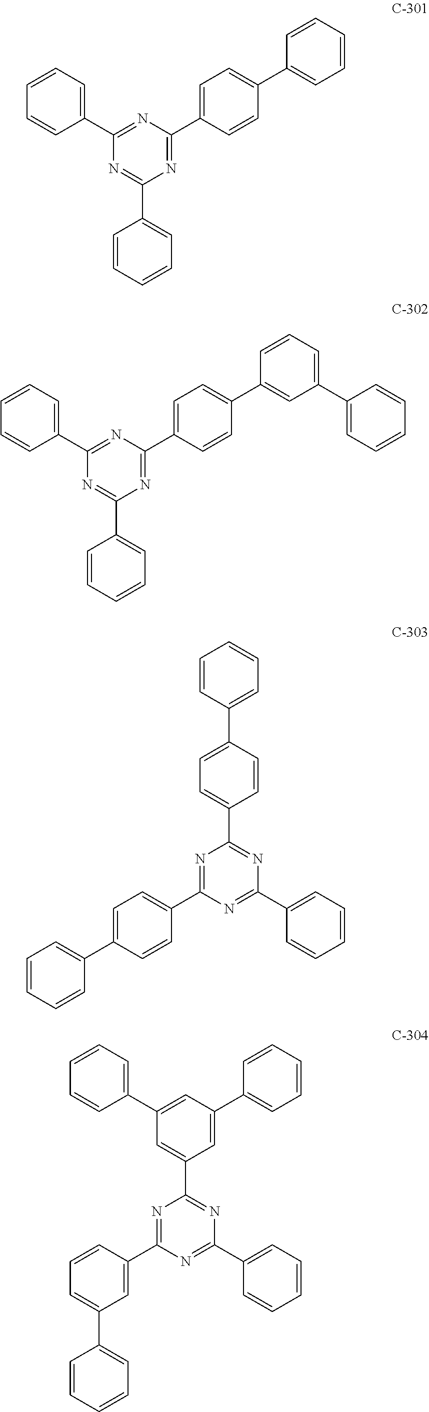 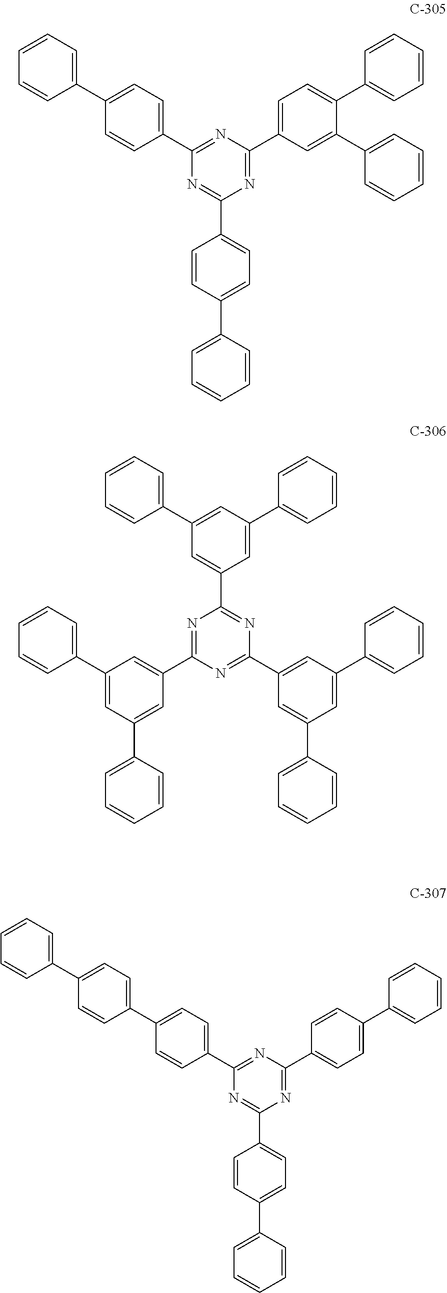 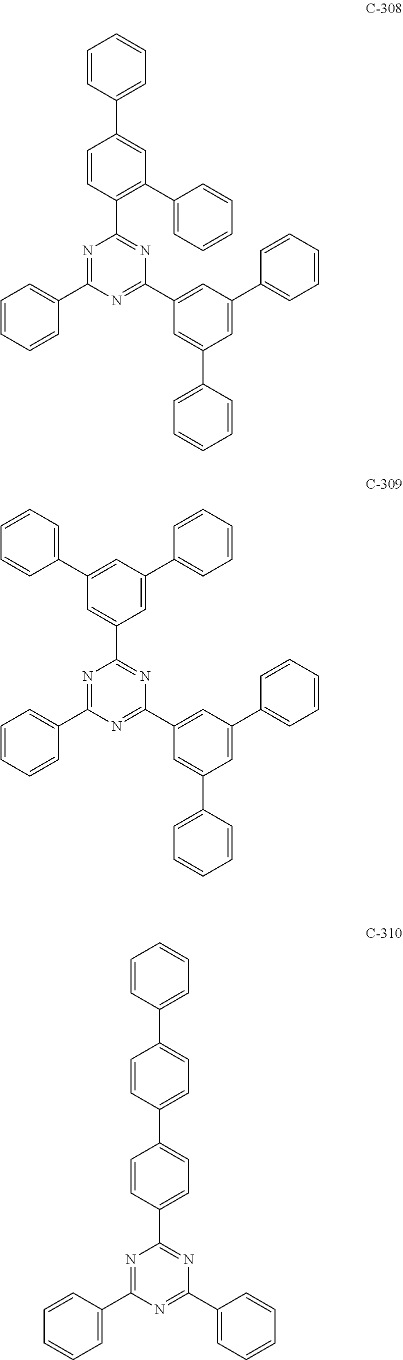 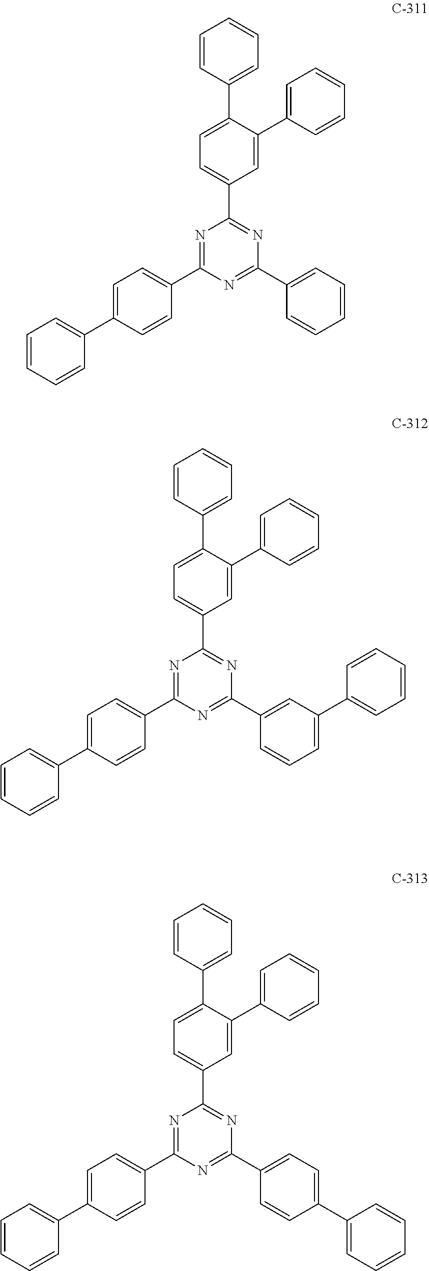 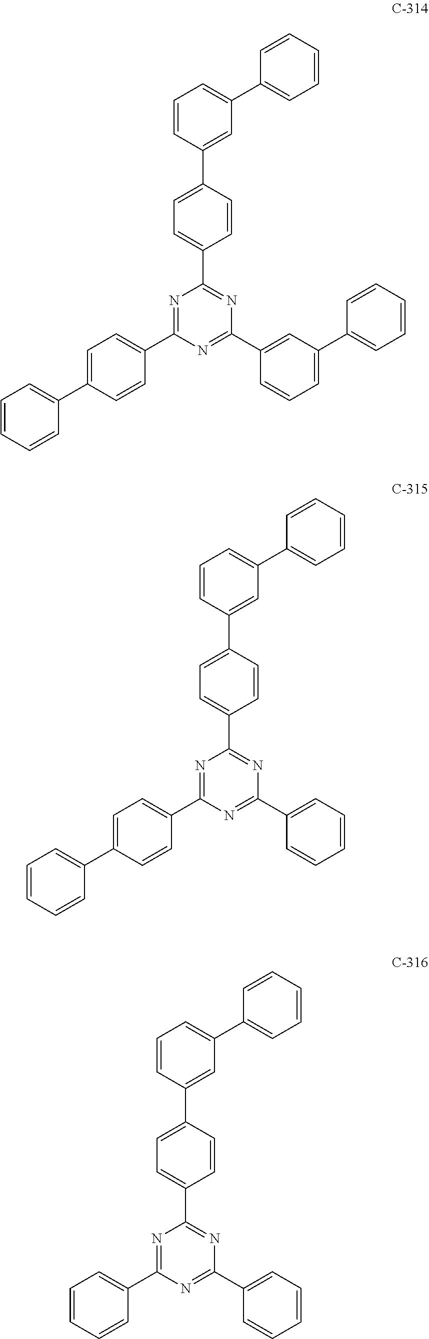 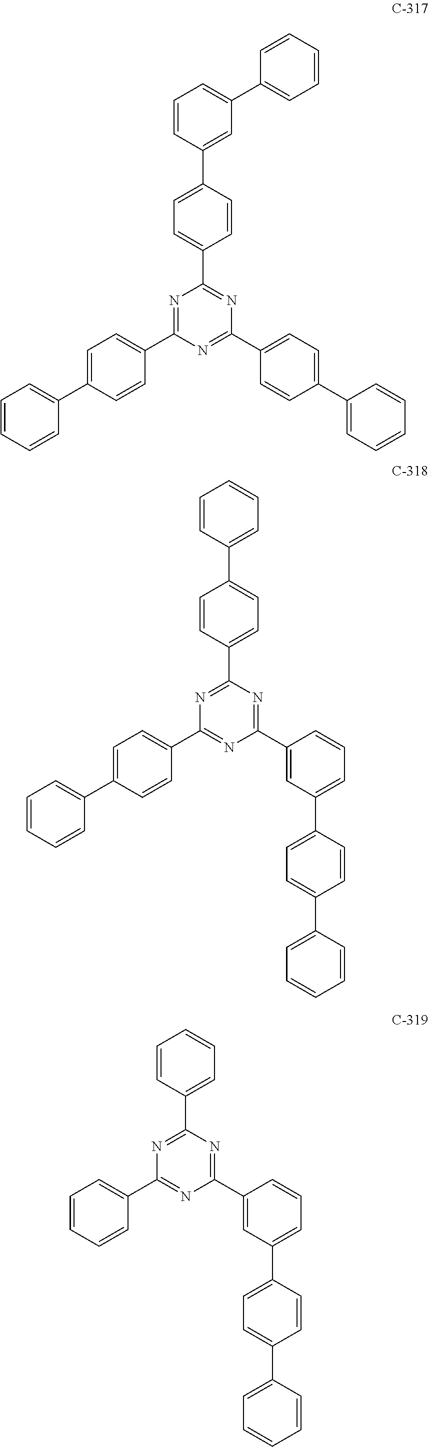 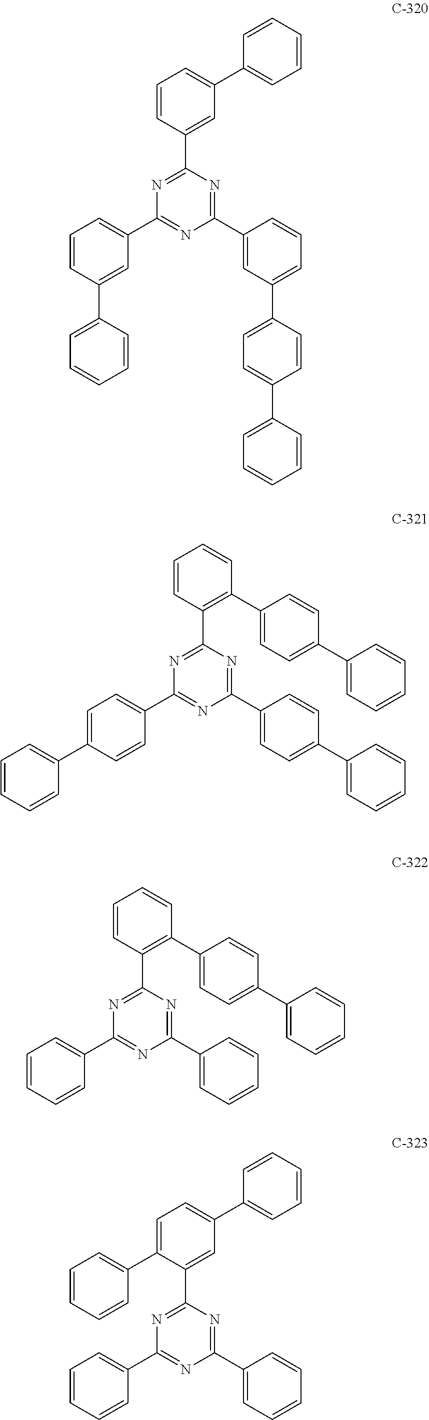 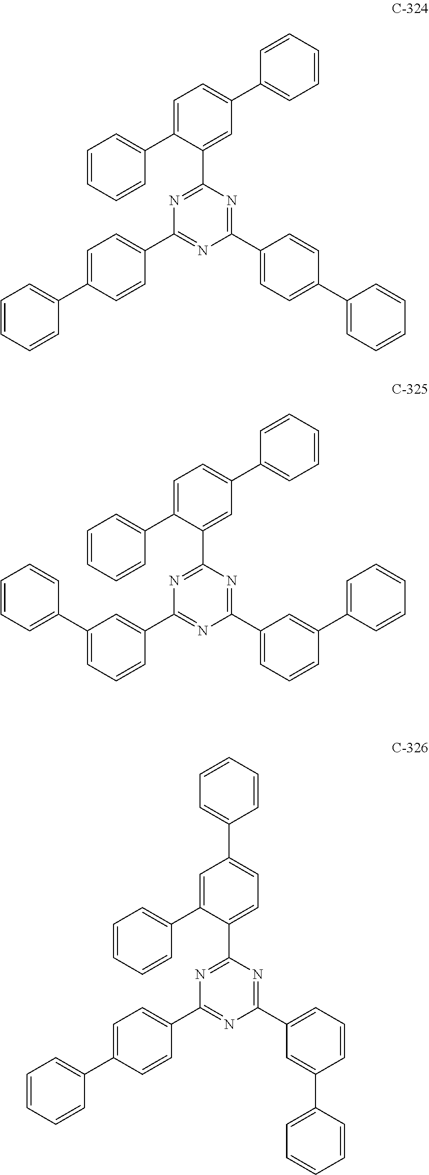 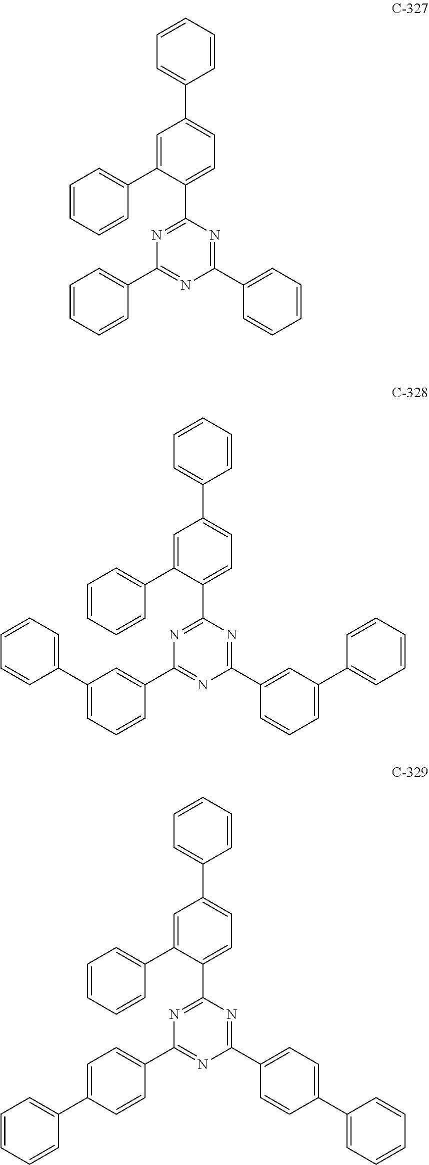 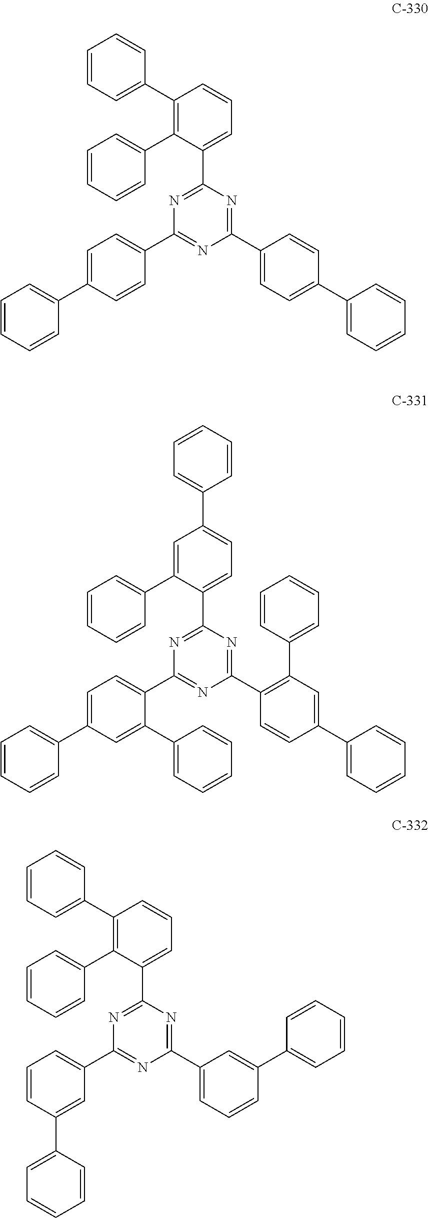 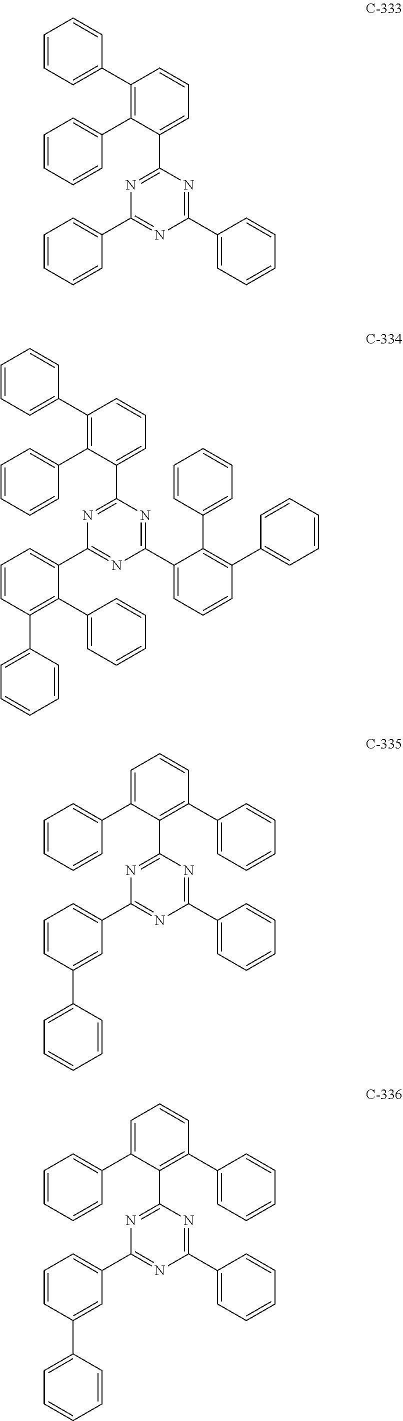 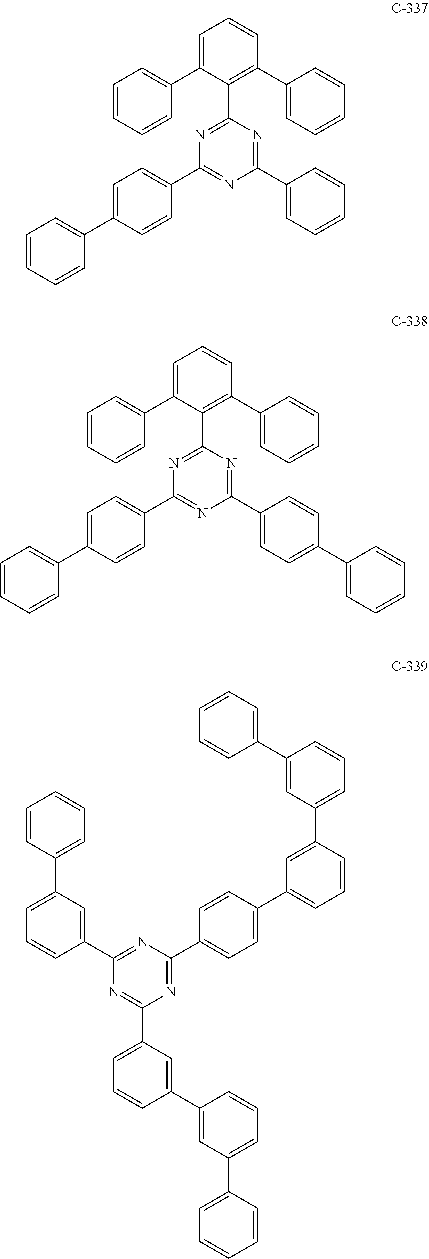 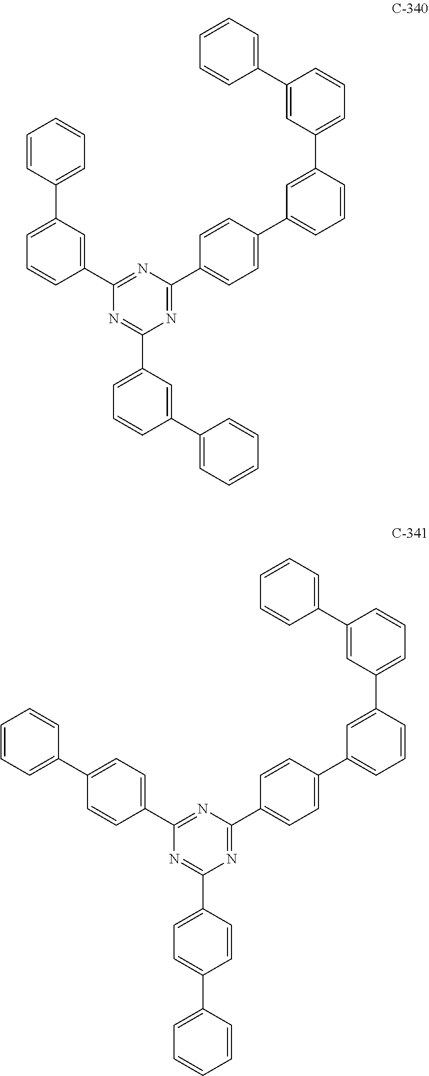 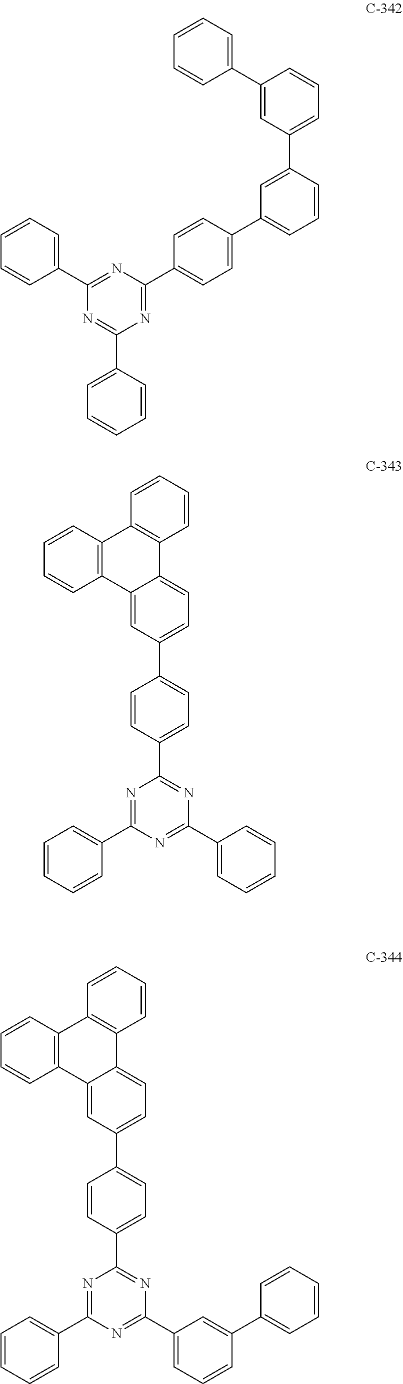 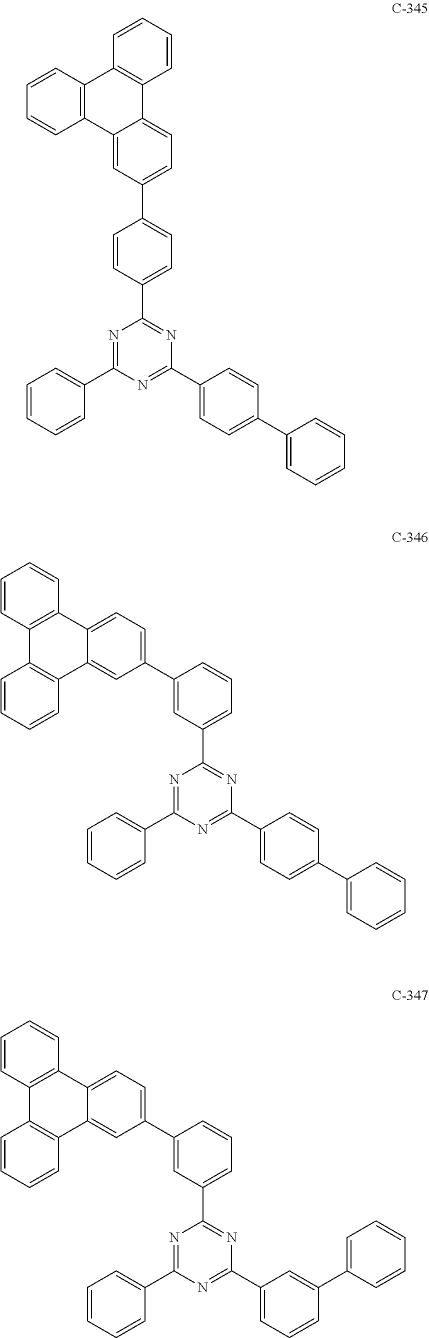 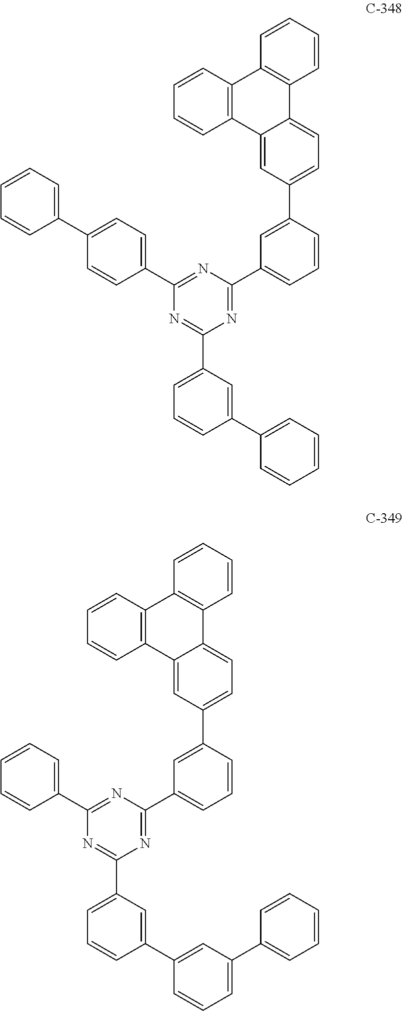 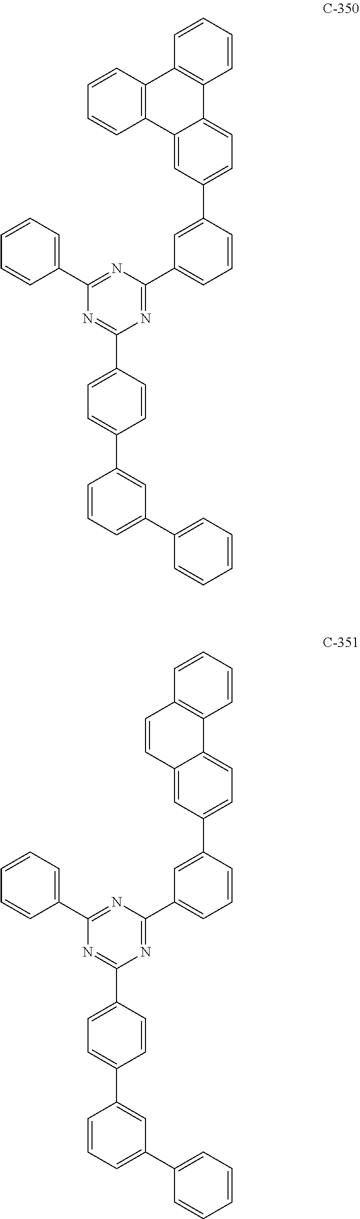 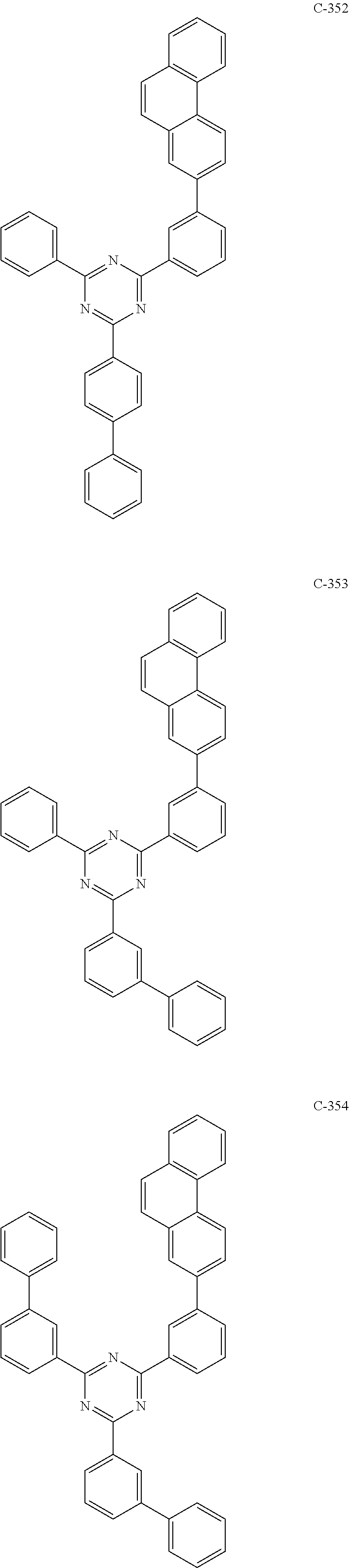 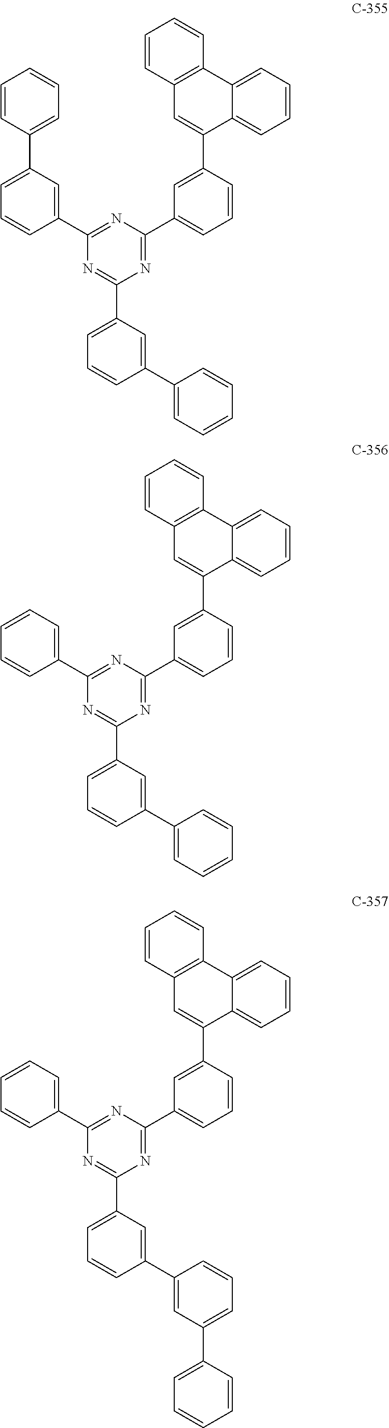 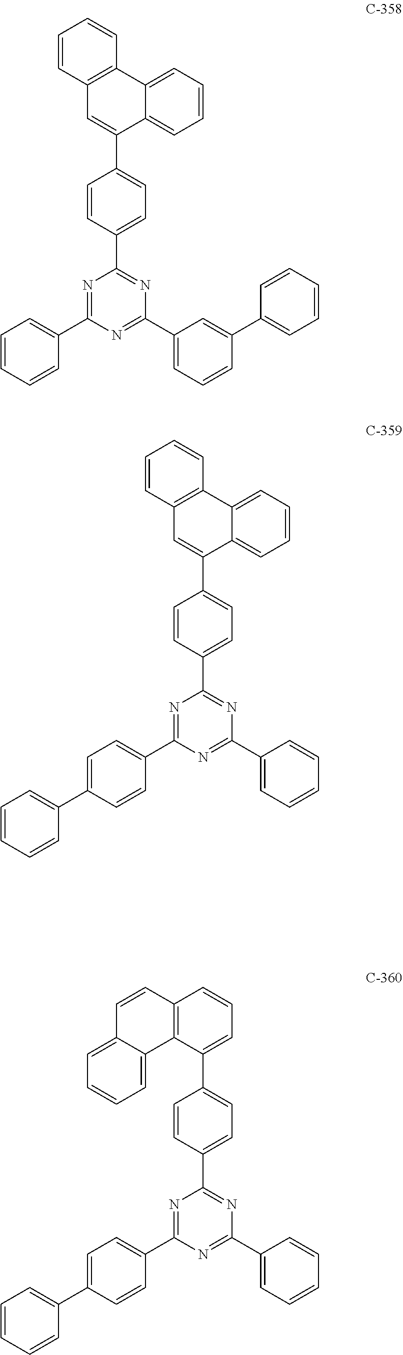 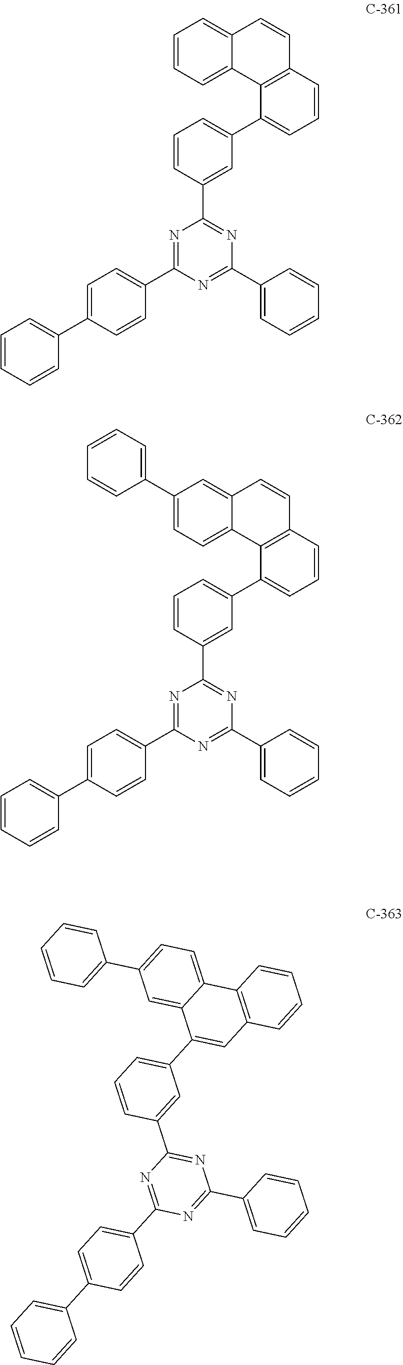 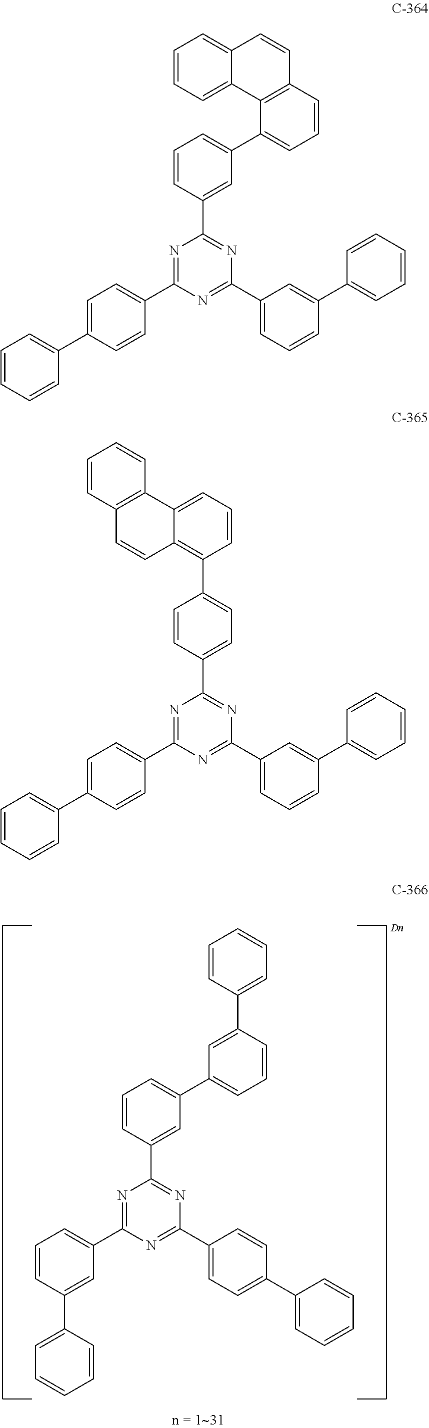 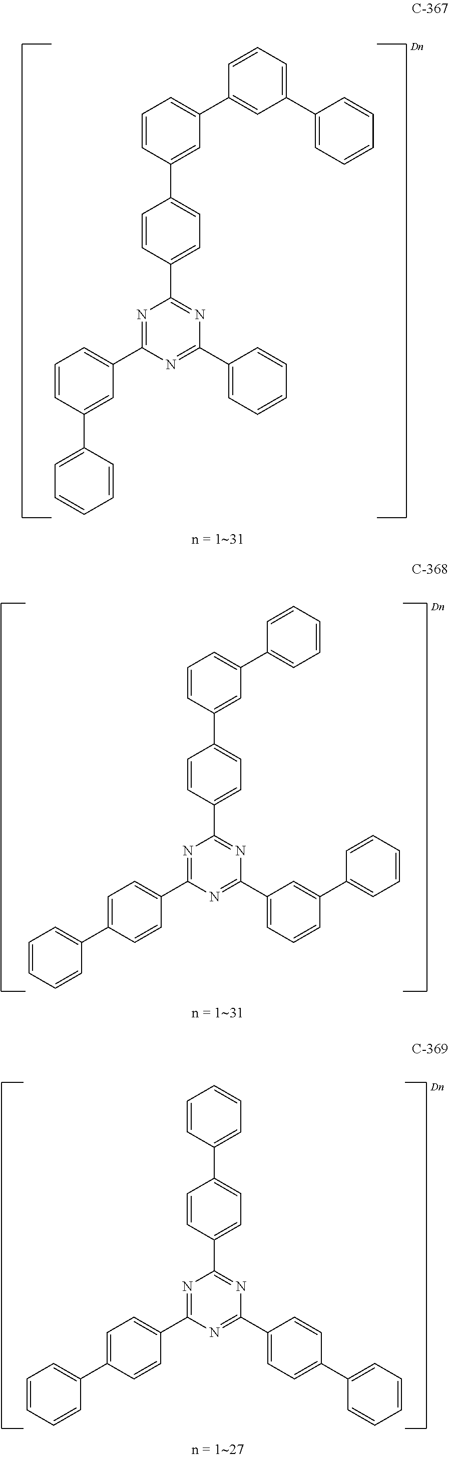 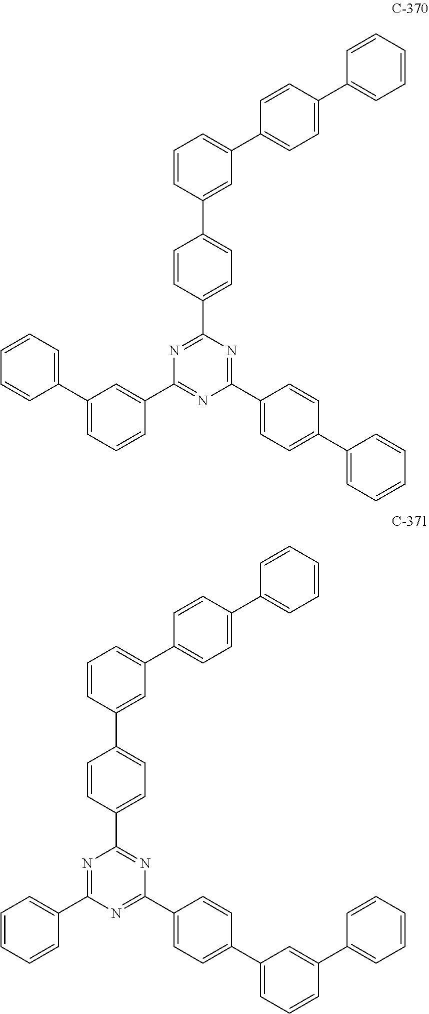 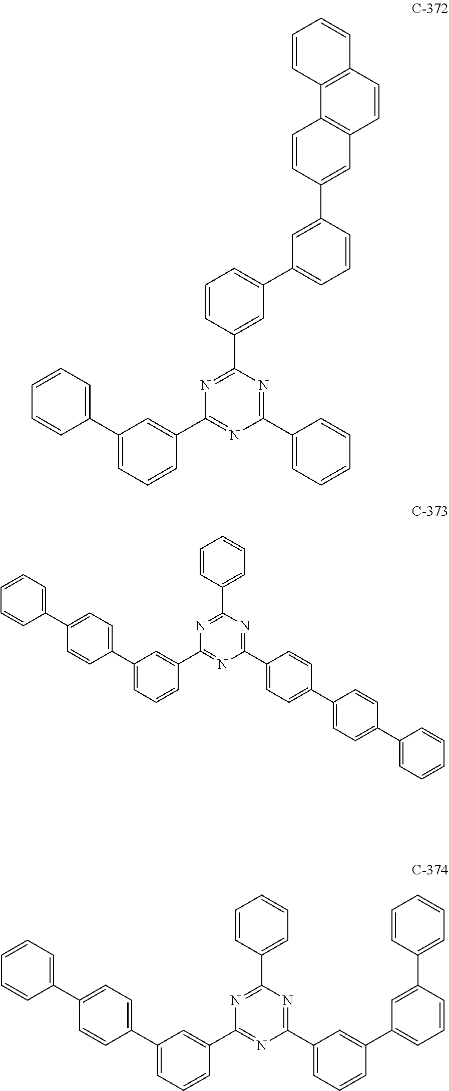 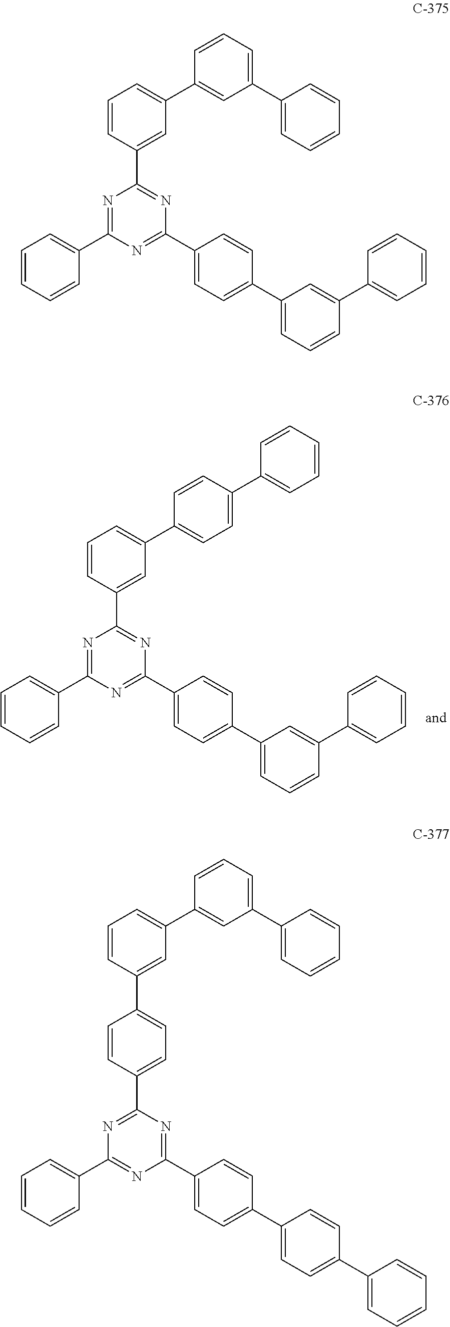
wherein Dn means that n number of hydrogen atoms are replaced with deuterium.

13. The plurality of host materials according to claim 1, wherein the compound represented by formula 2 is at least one selected from the following compounds: 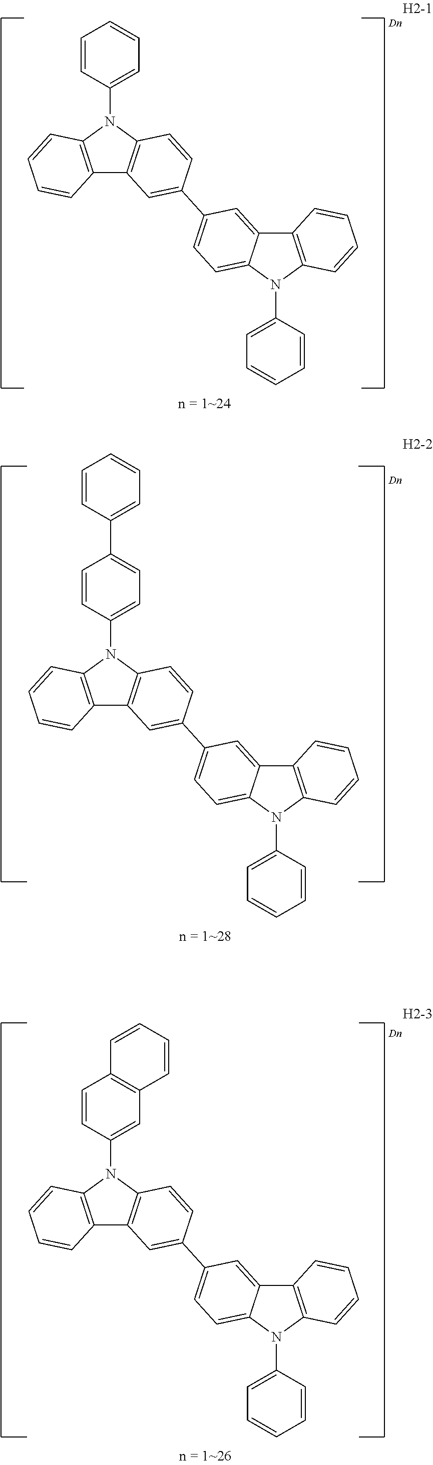 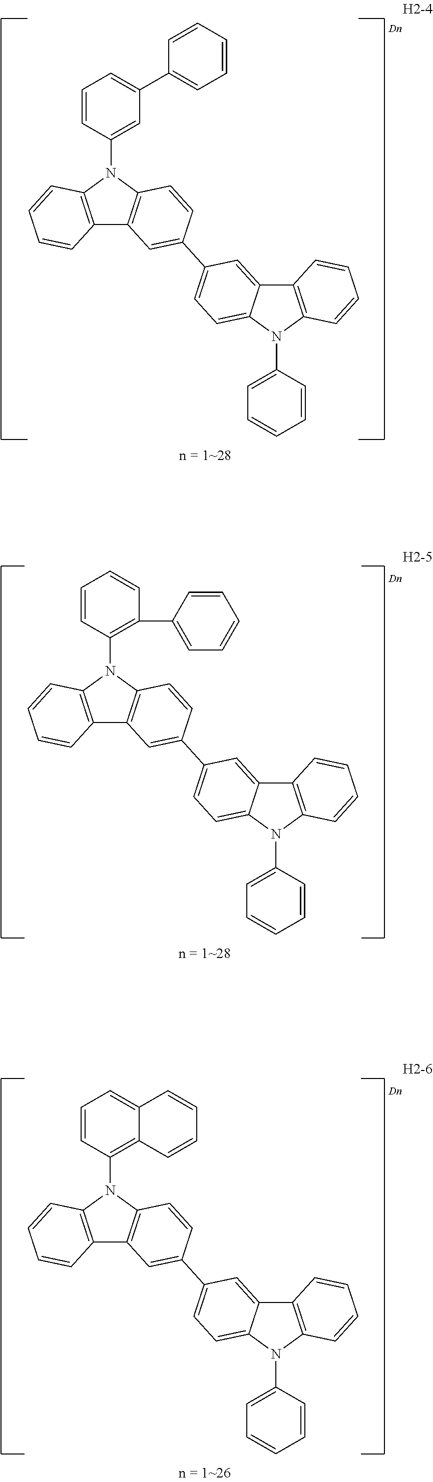 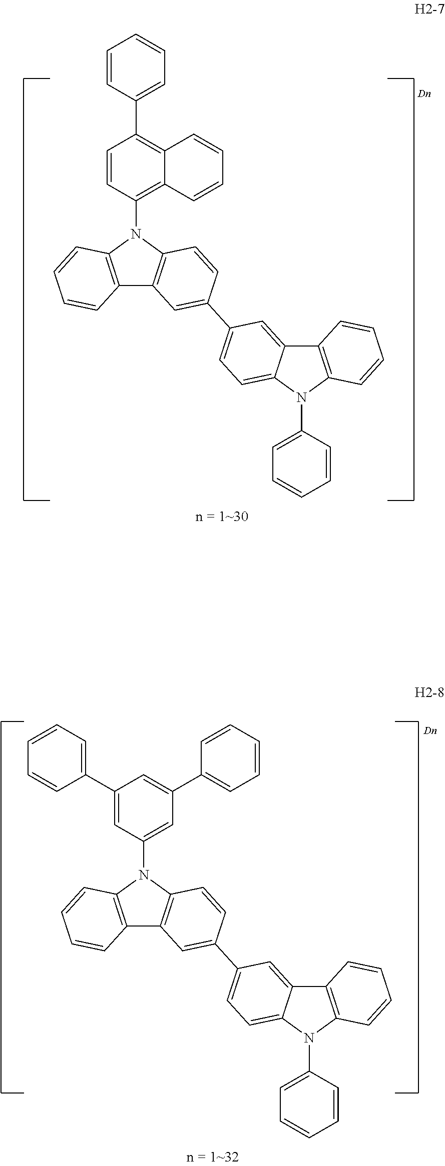 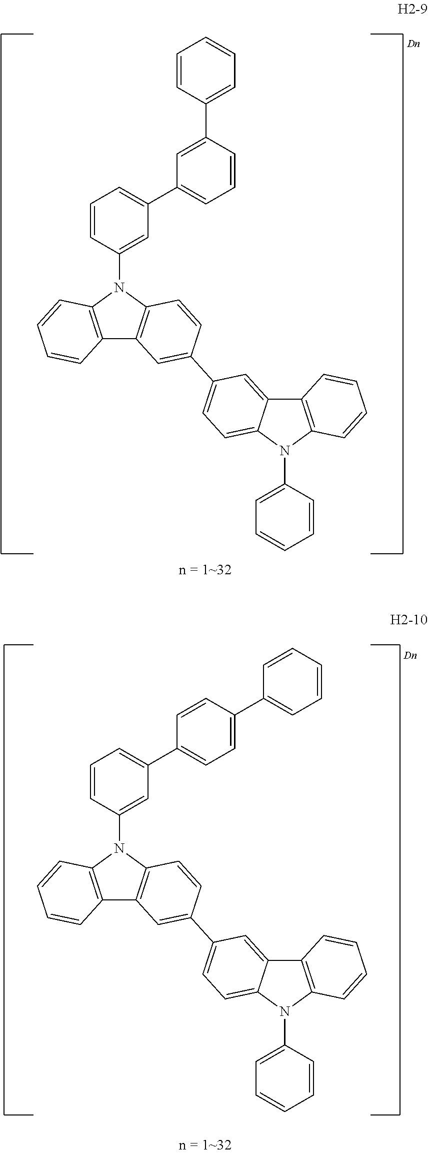 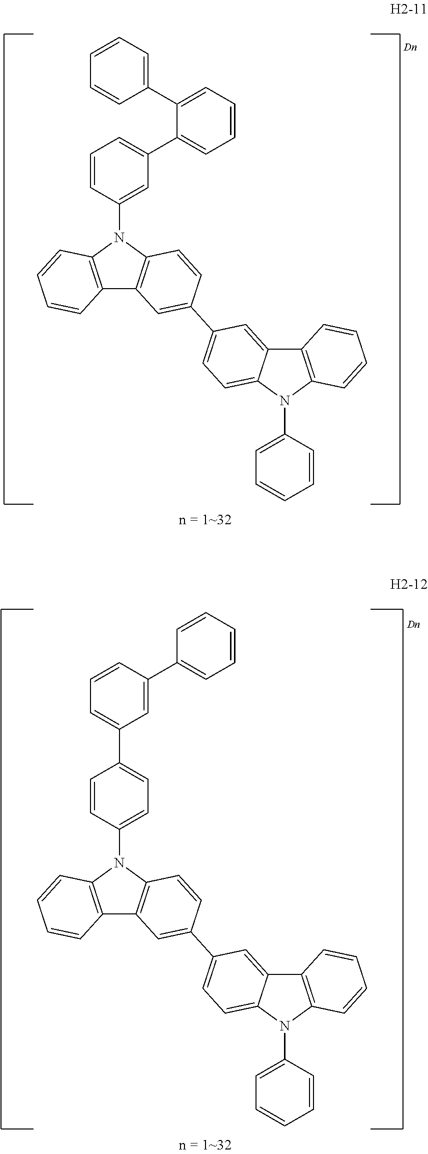 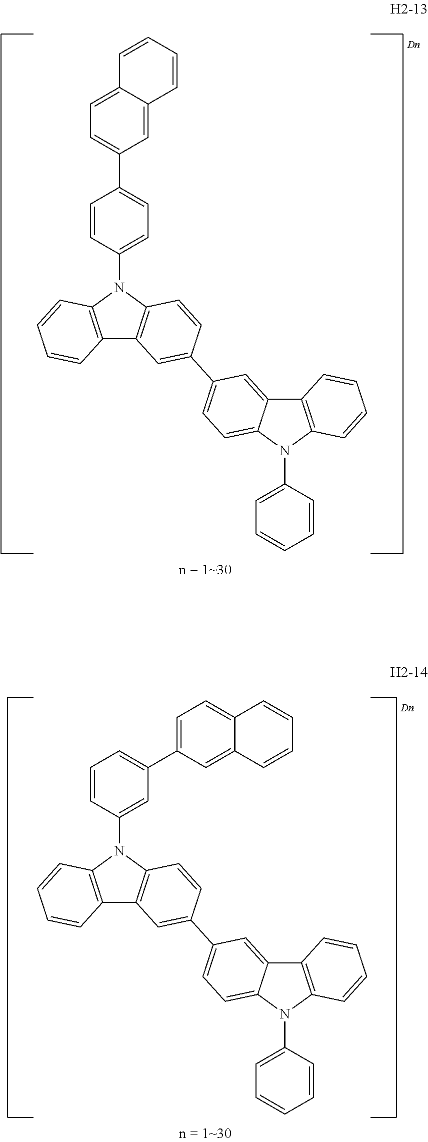 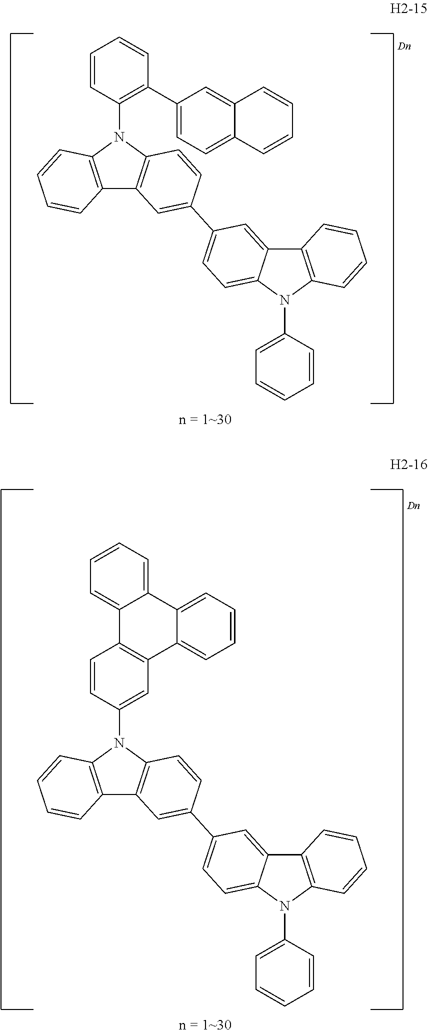 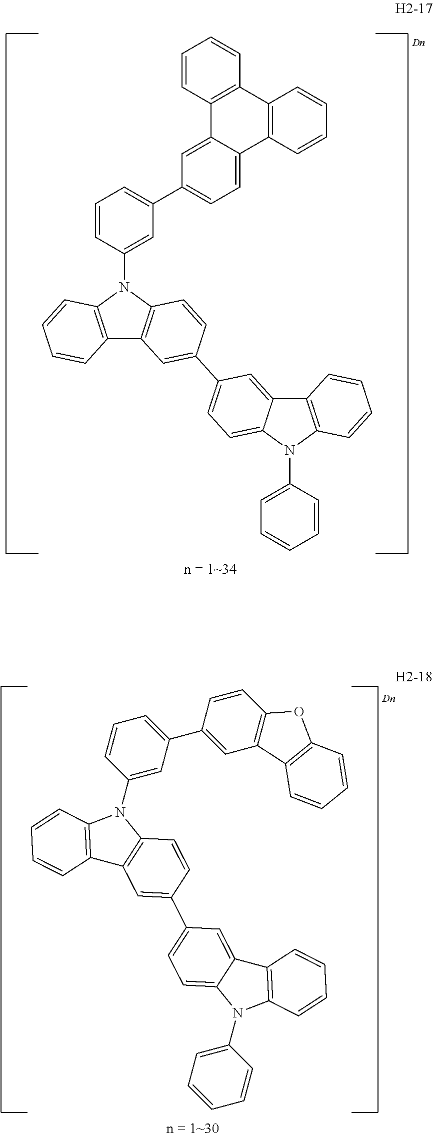 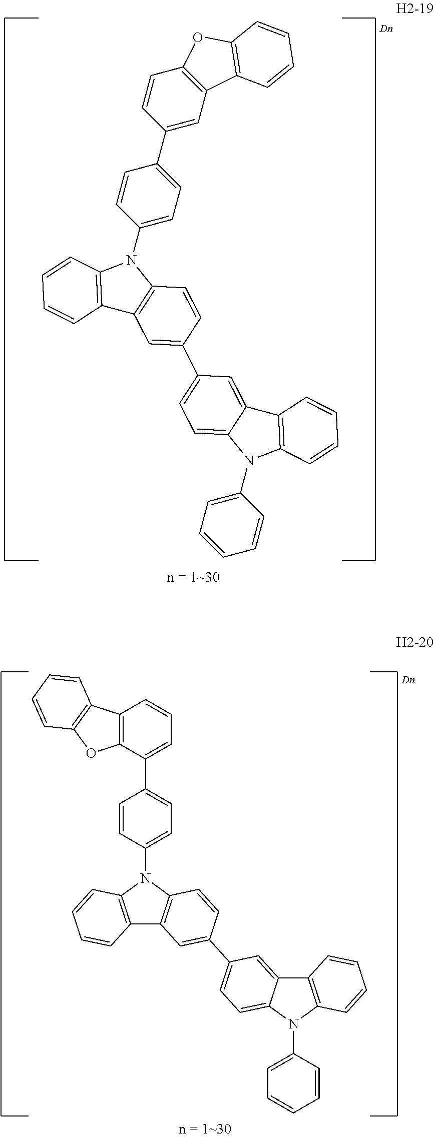 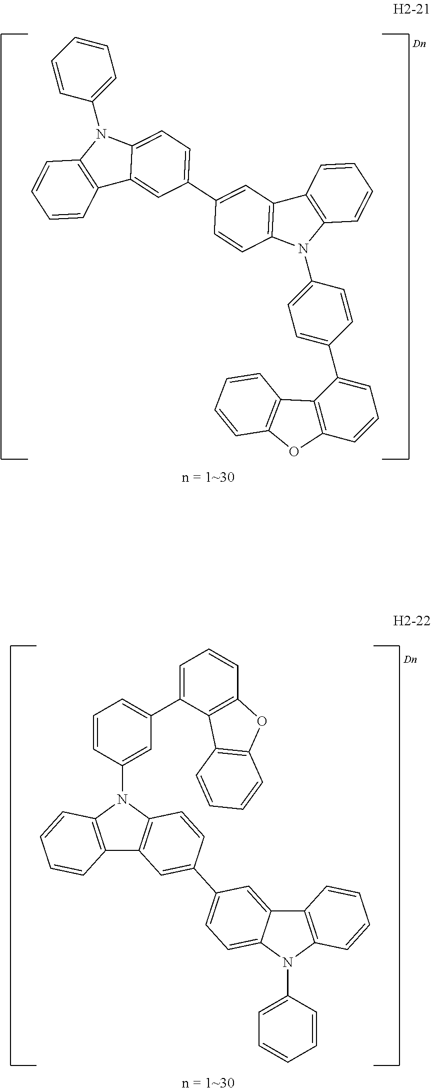 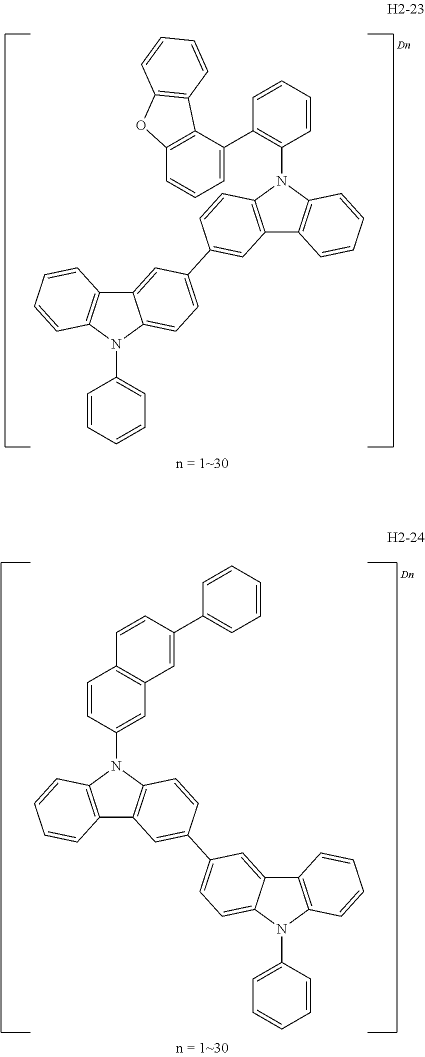 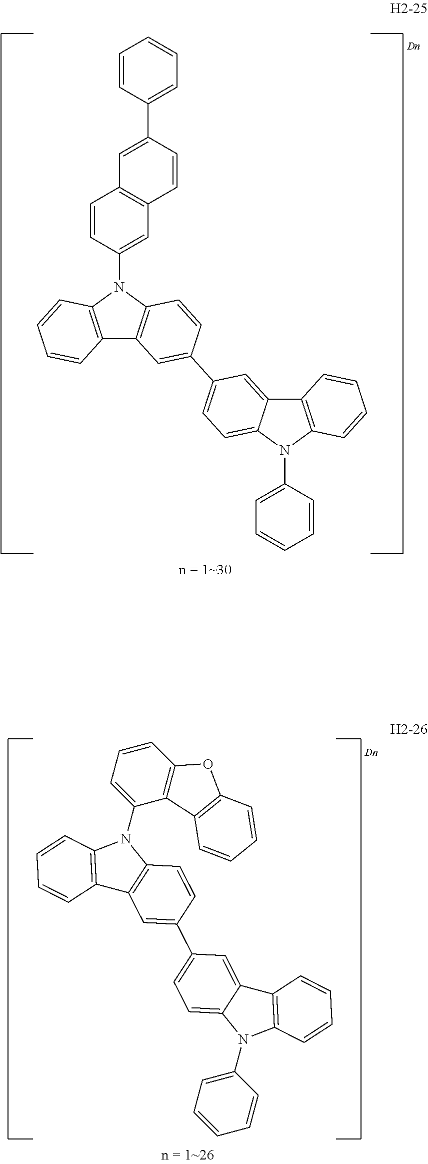 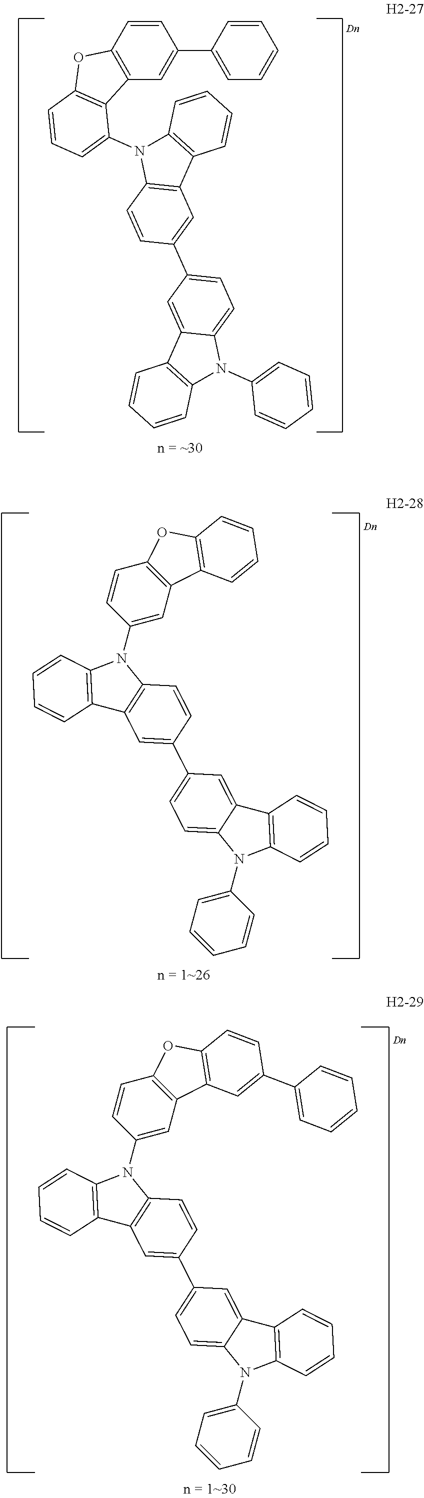 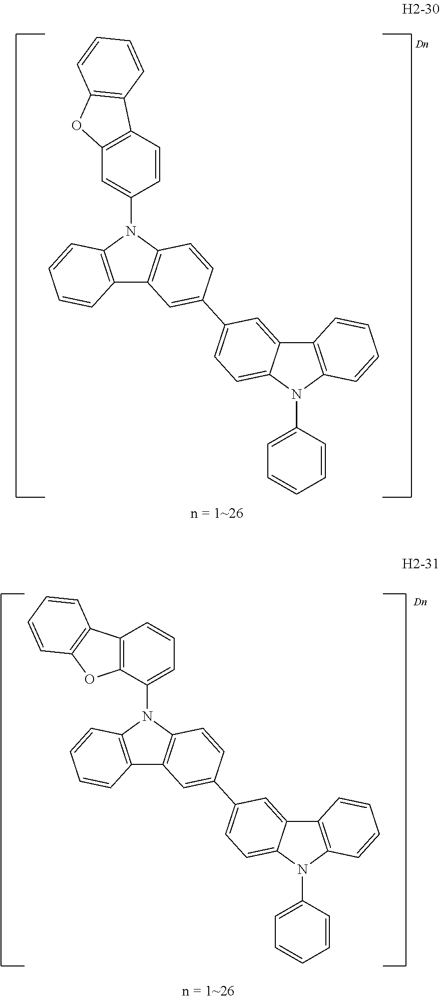 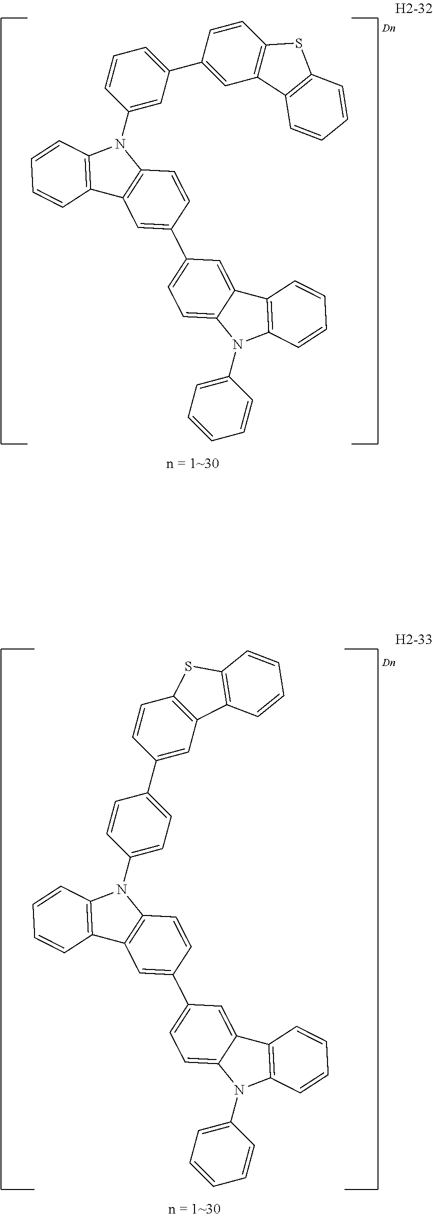 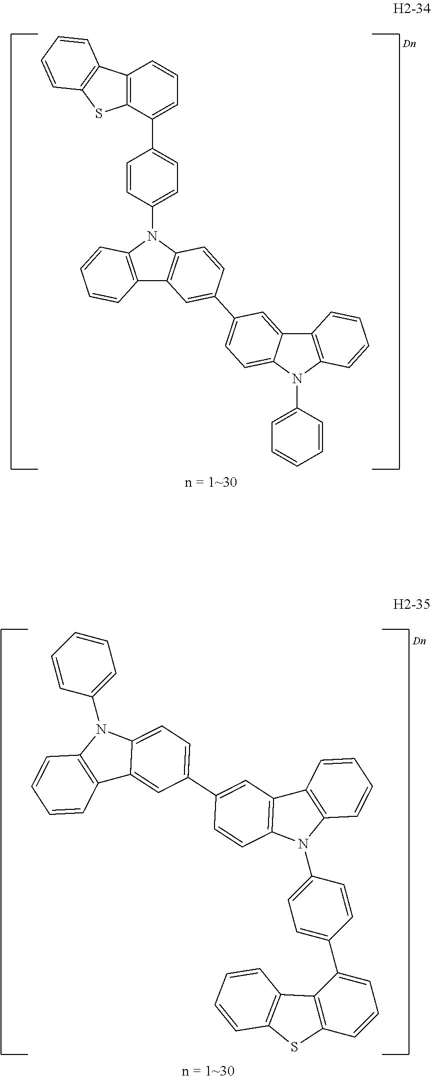 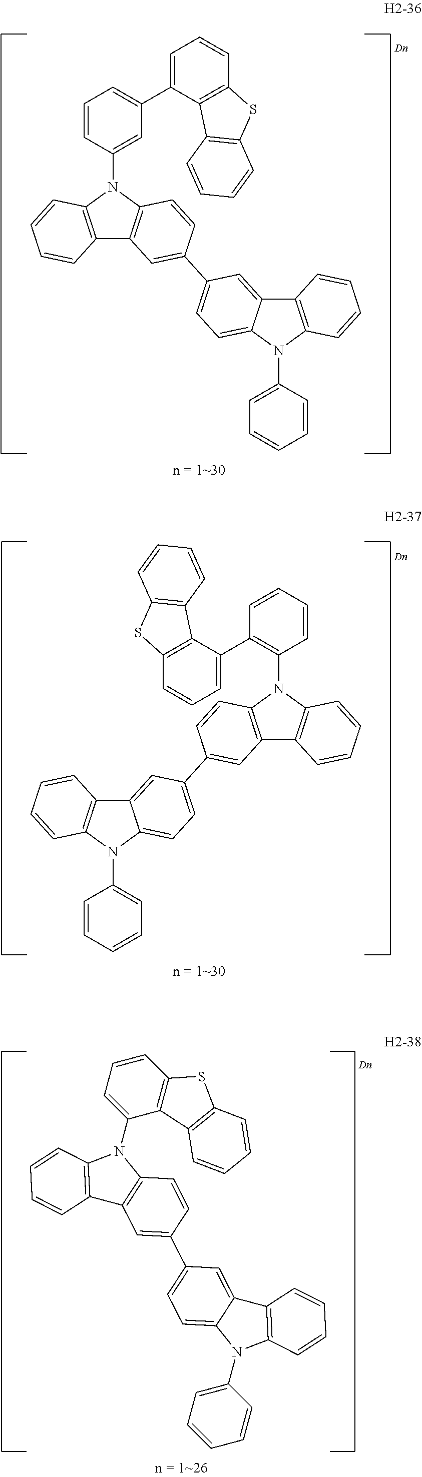 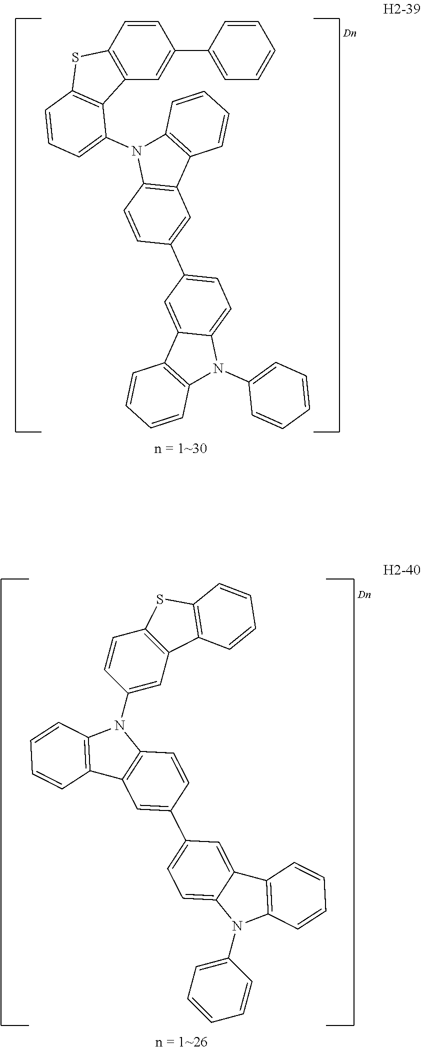 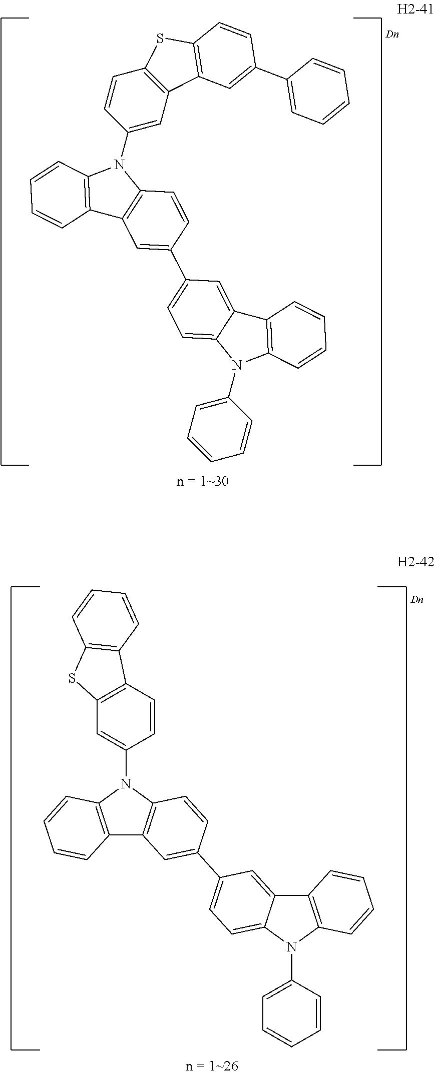 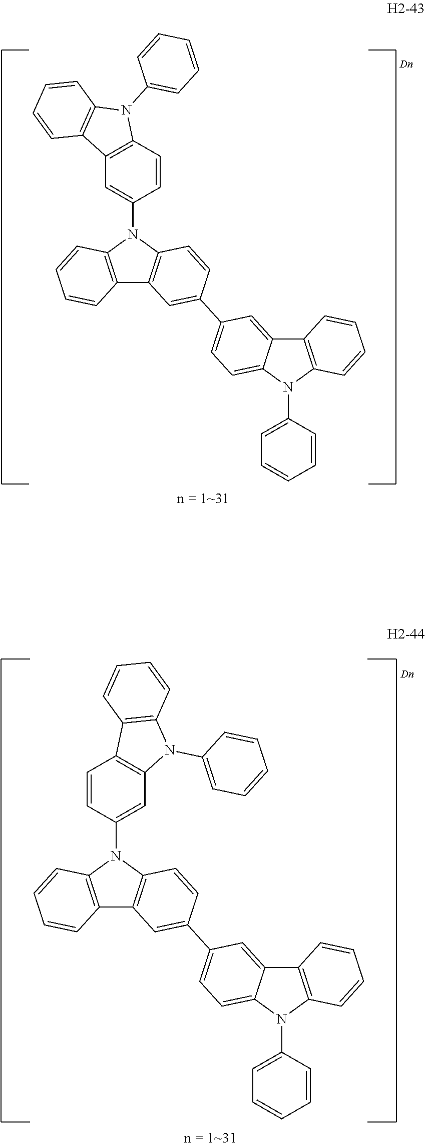 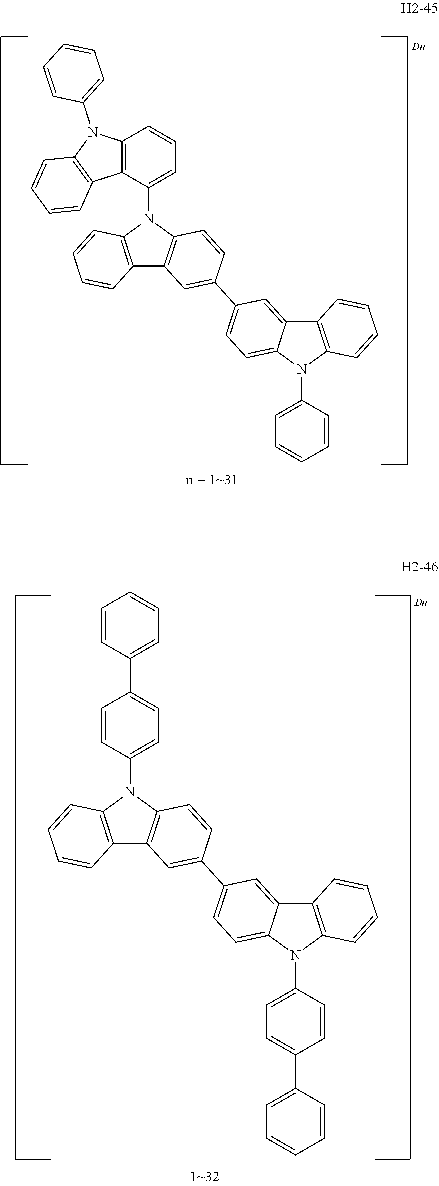 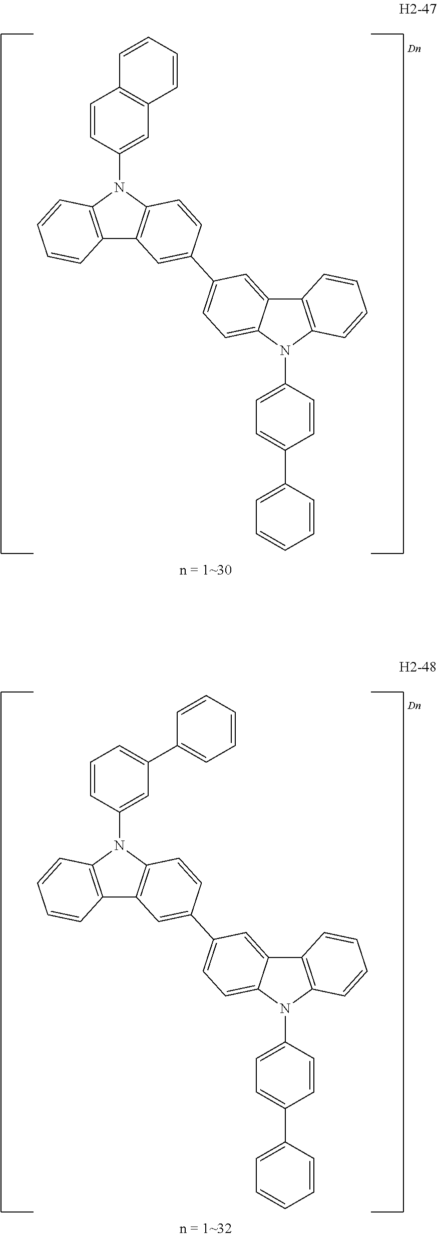 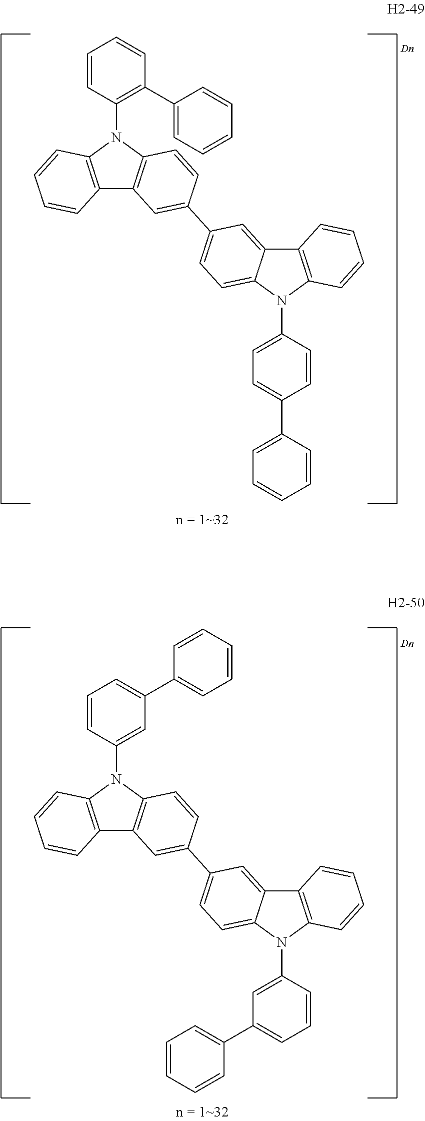 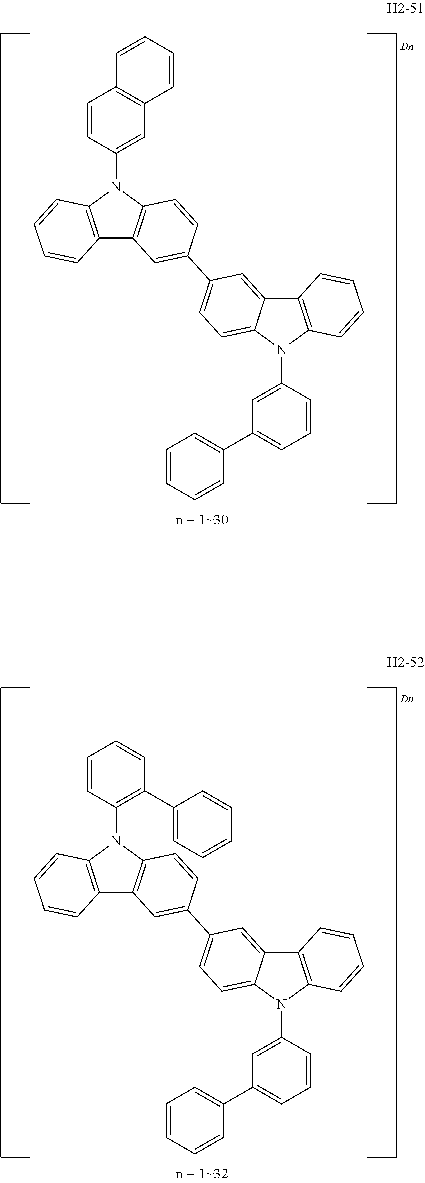 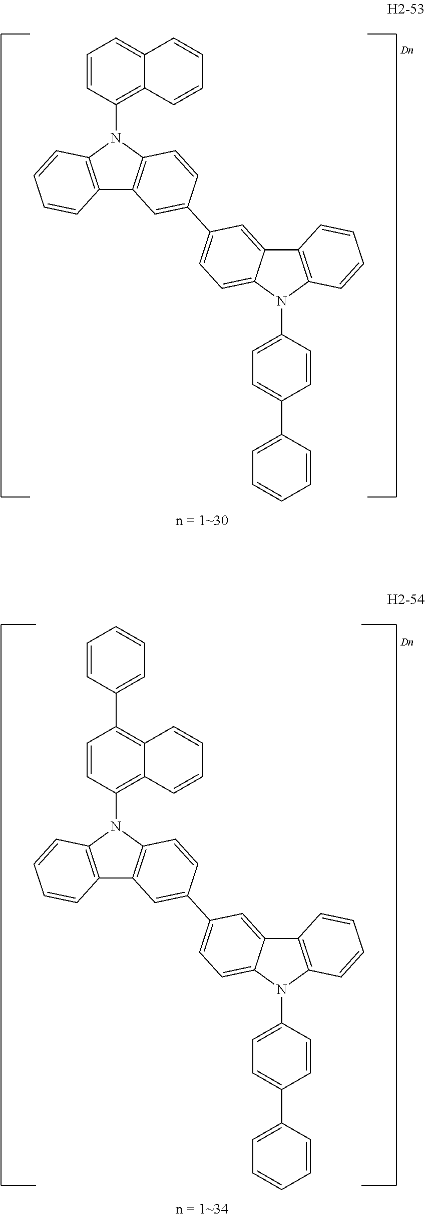 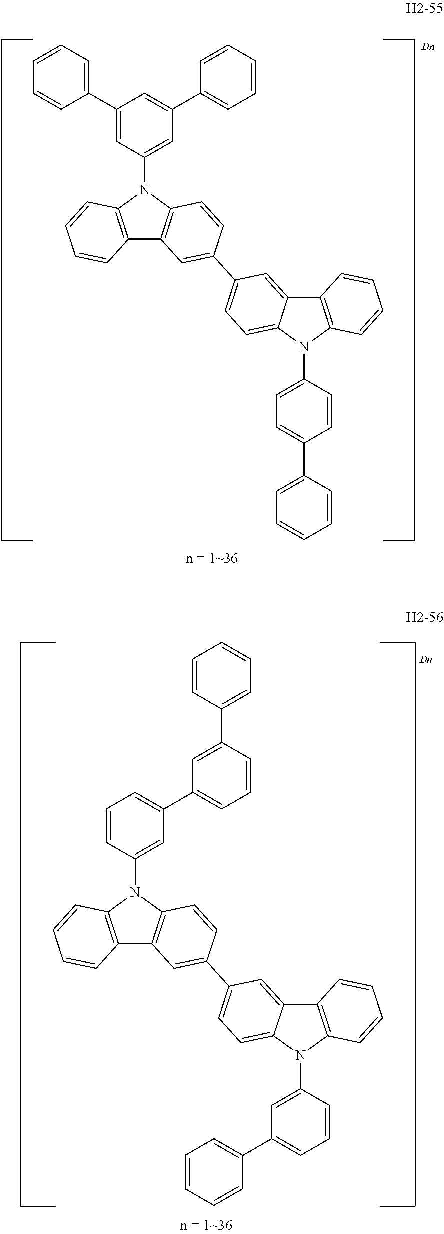 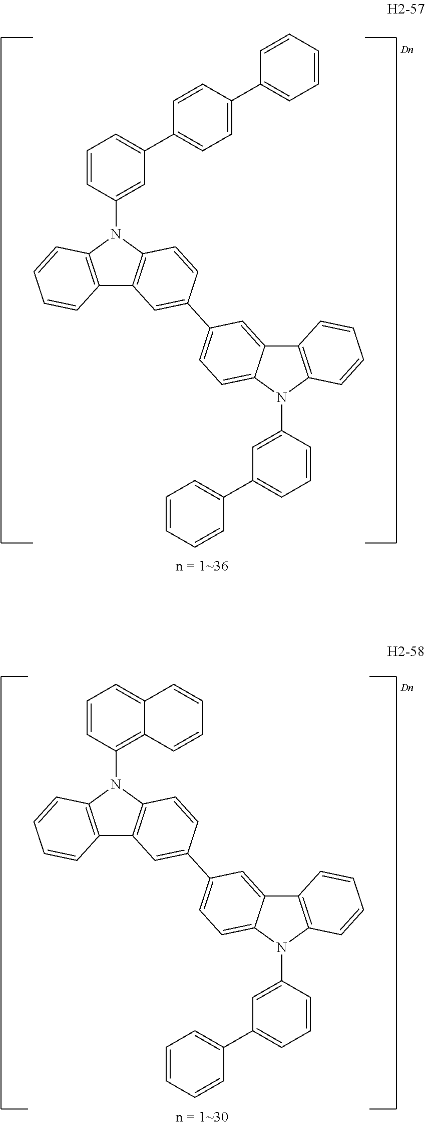 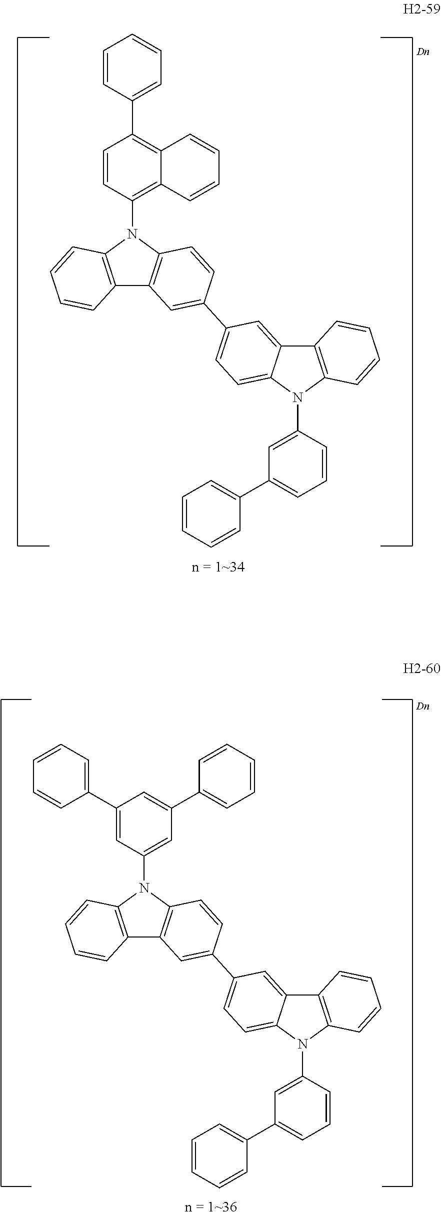 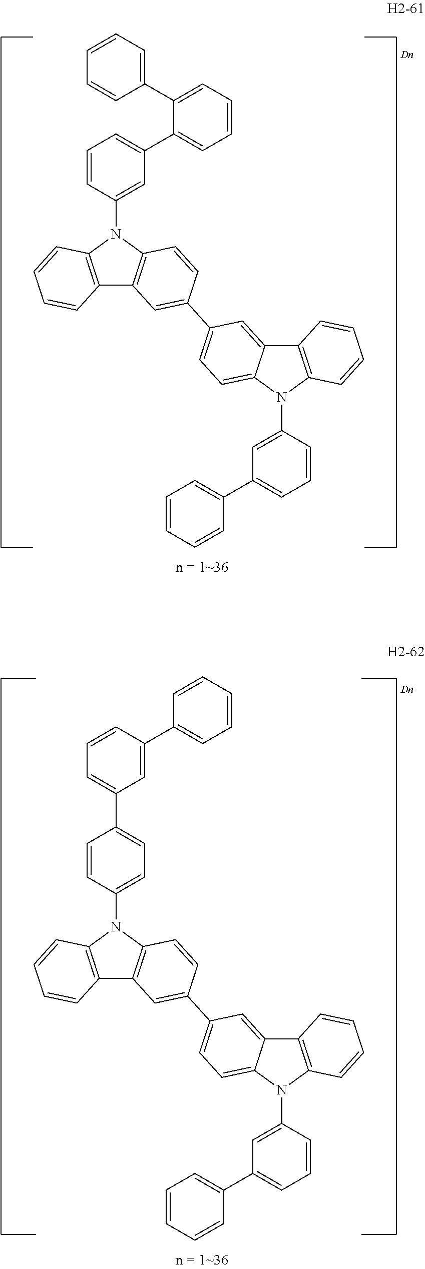 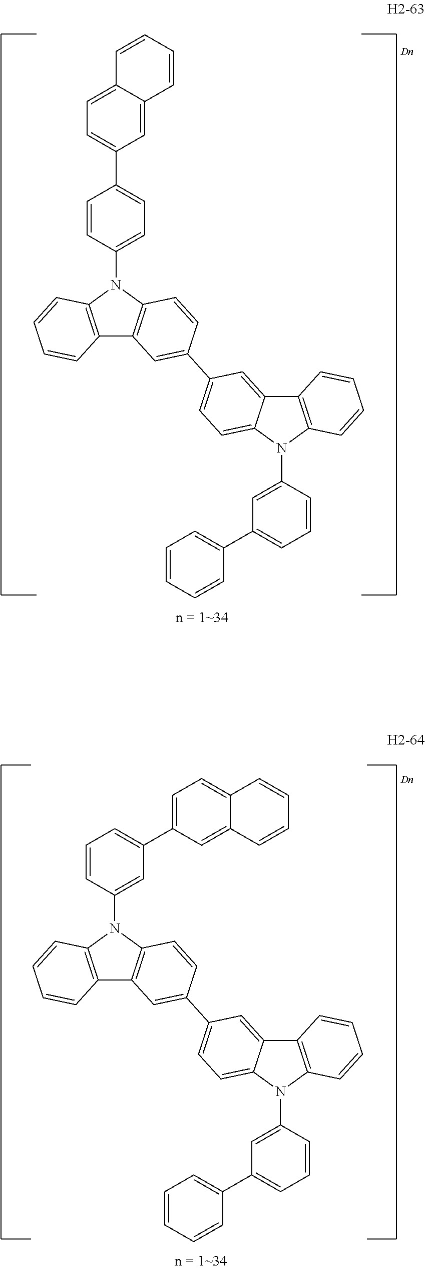 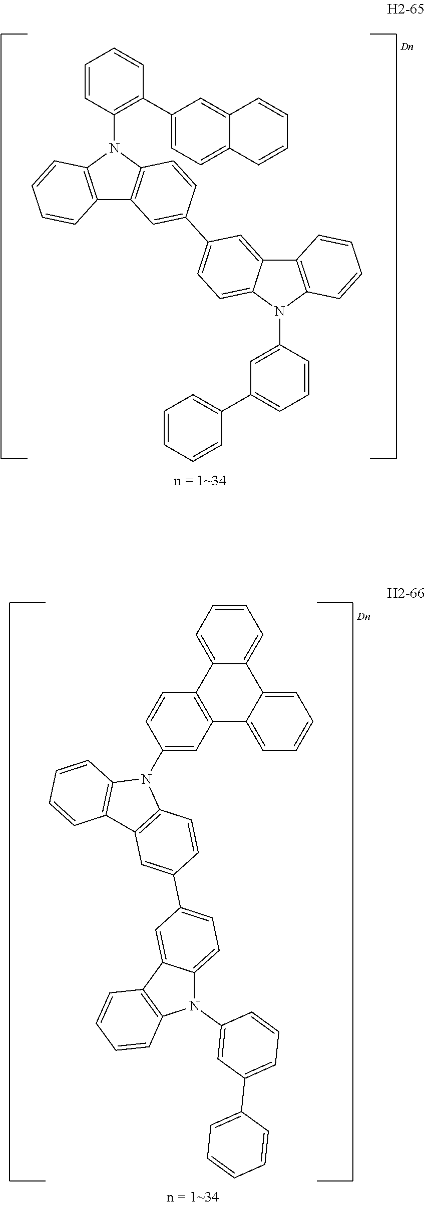 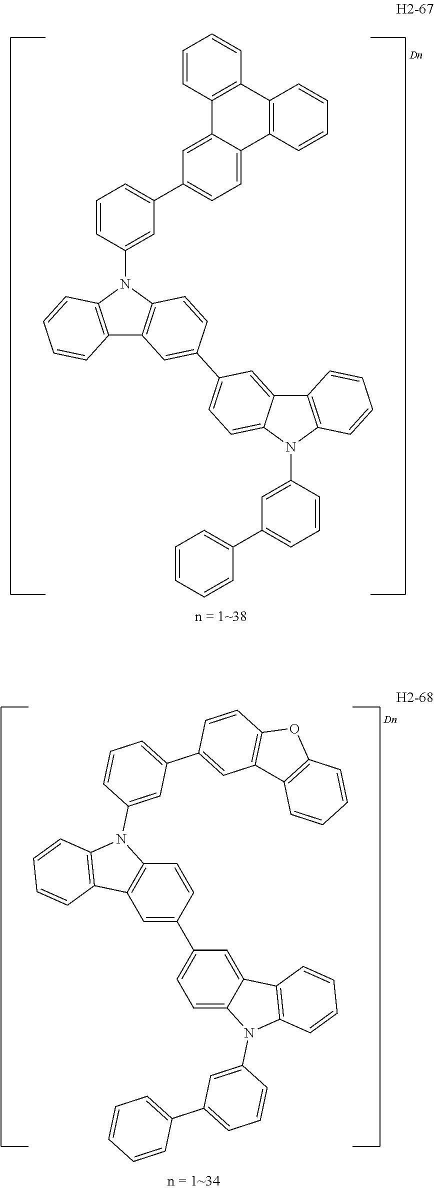 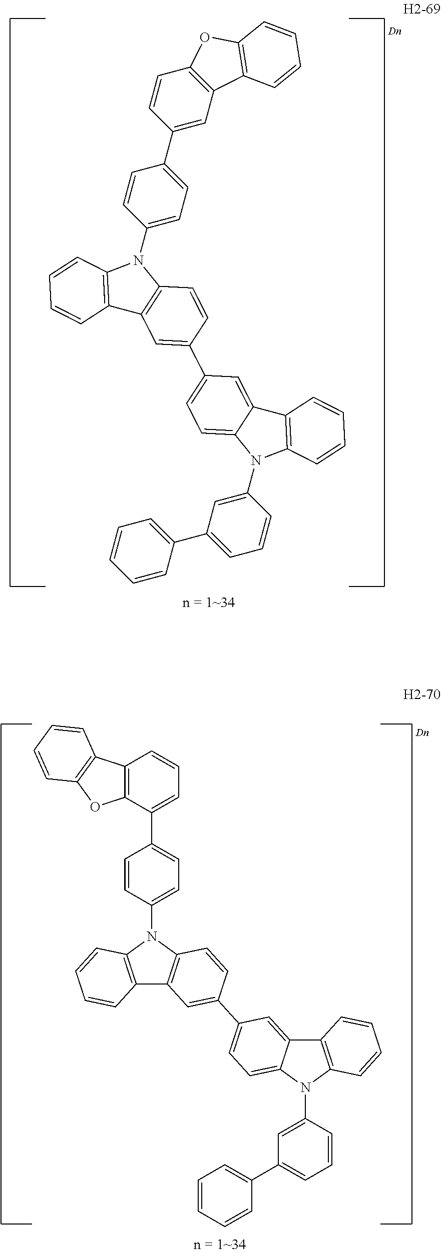 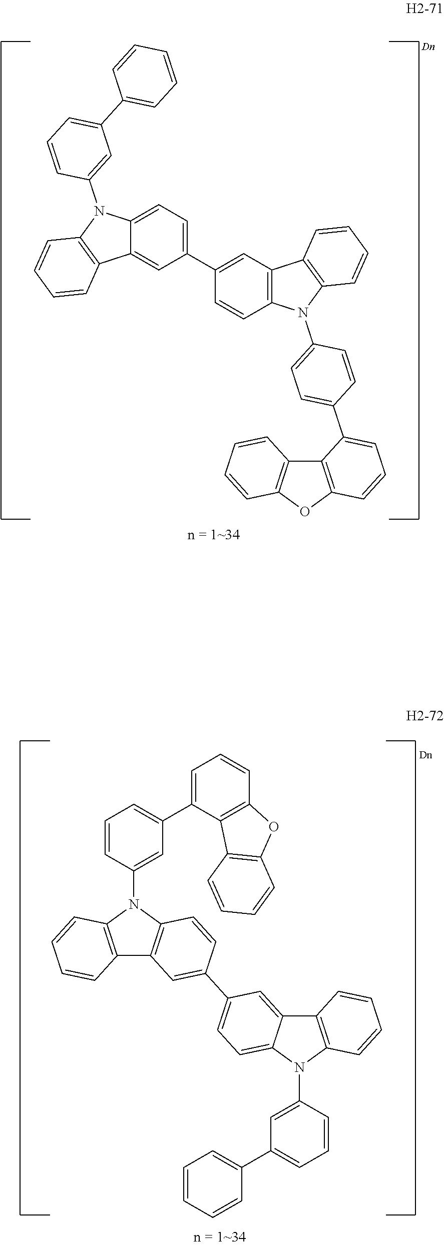 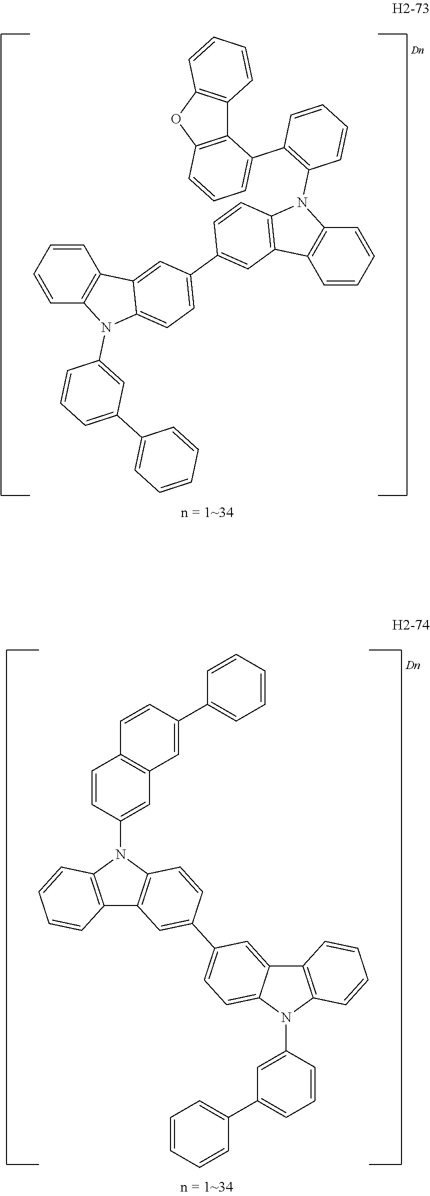 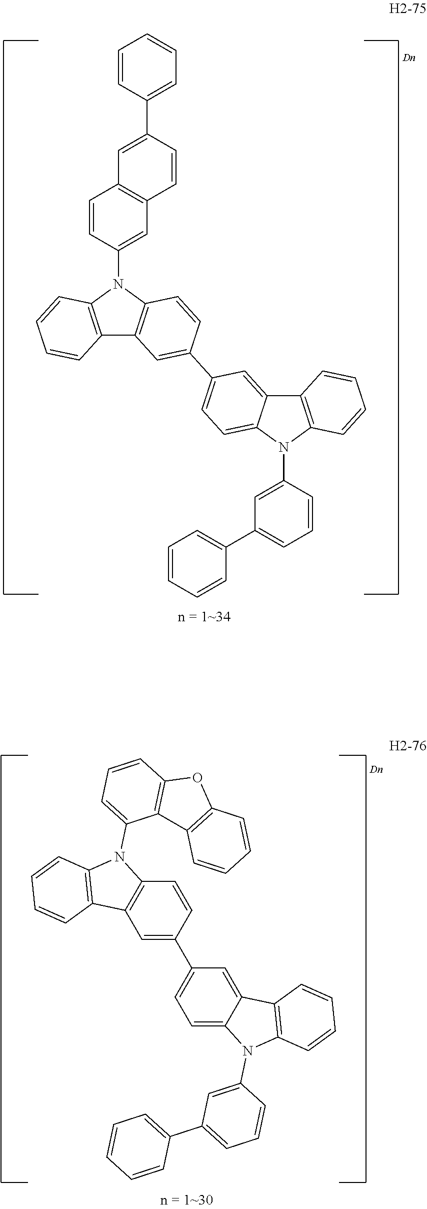 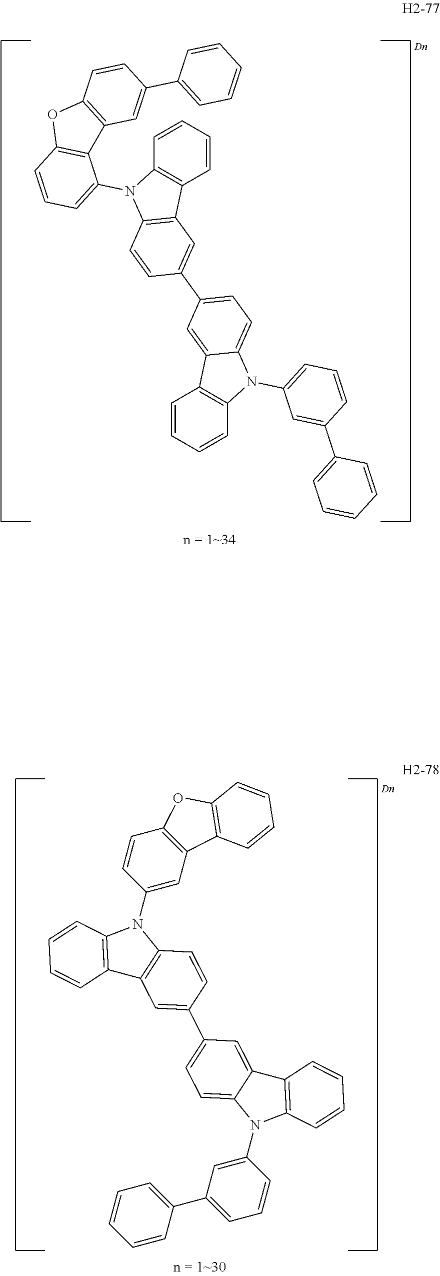 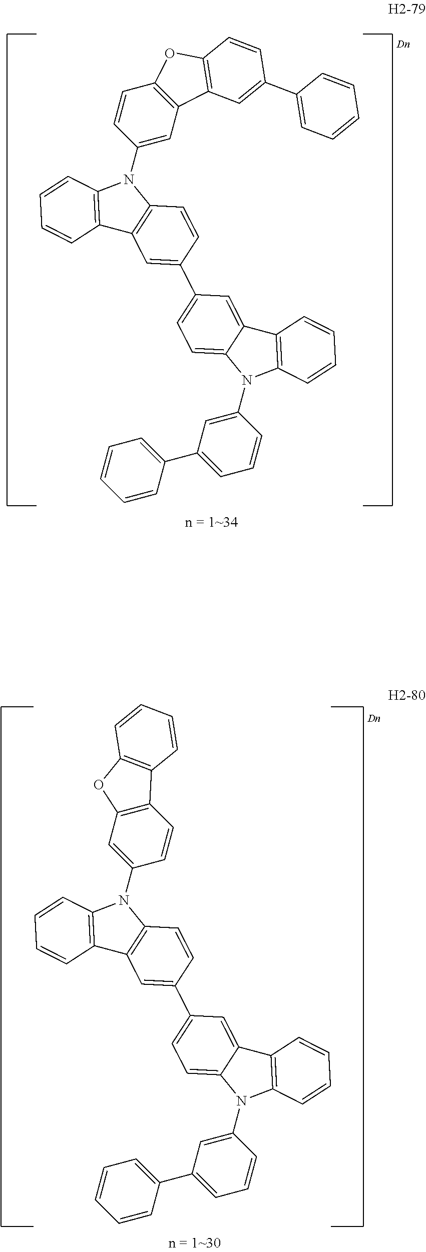 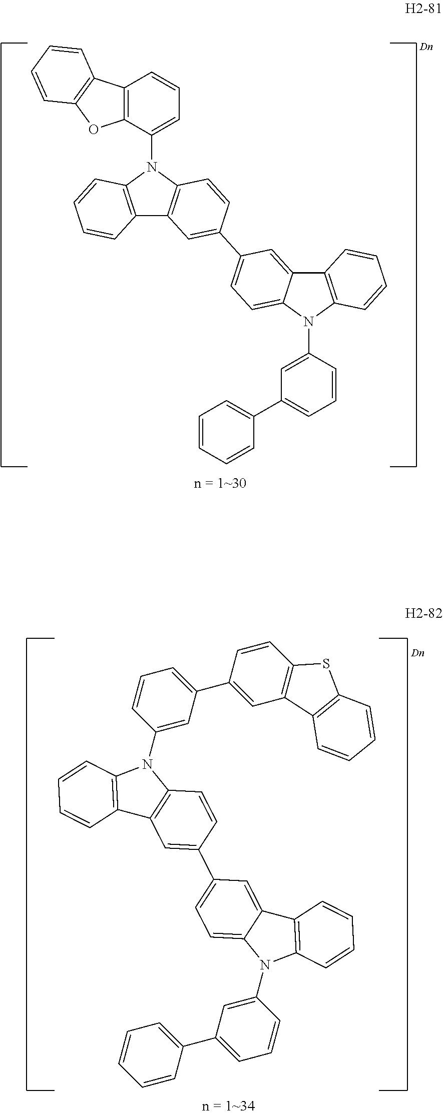 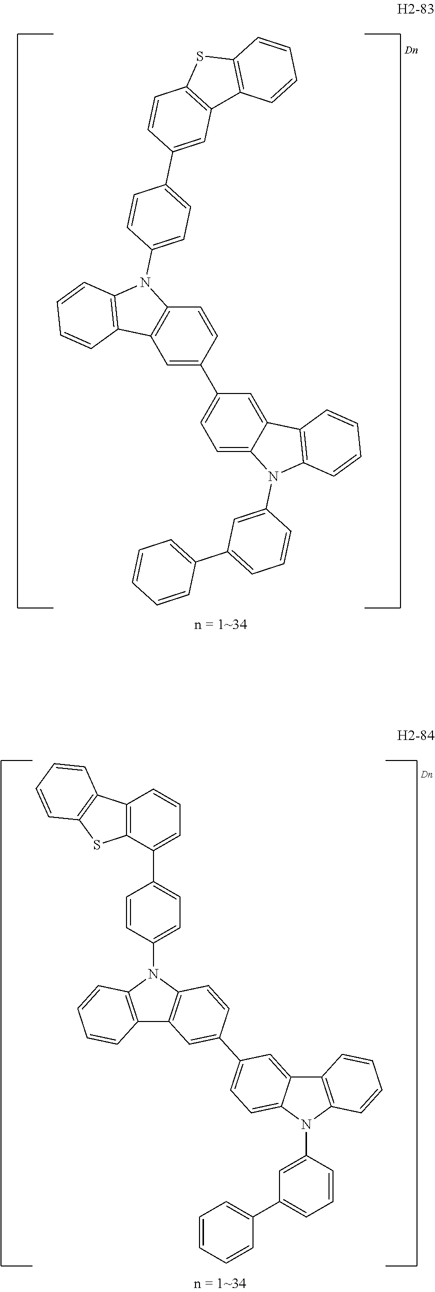 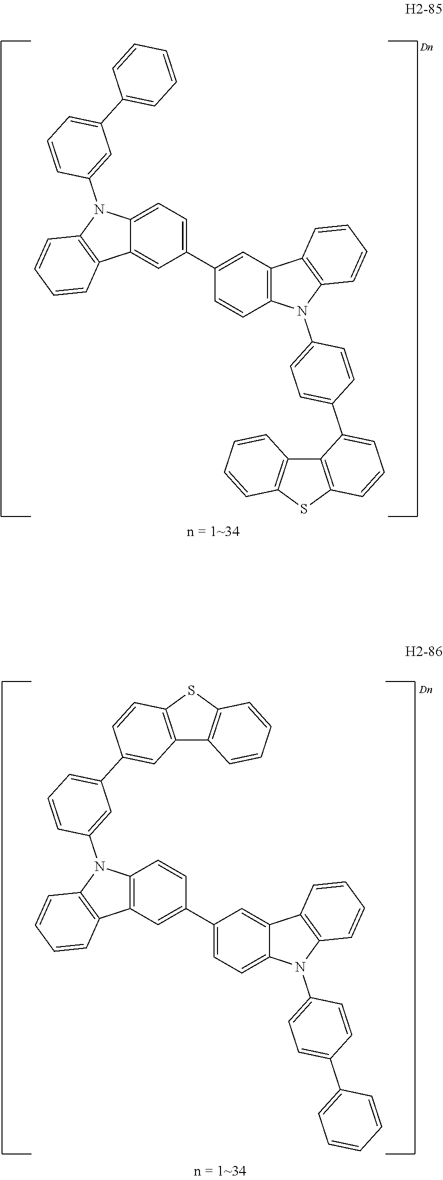 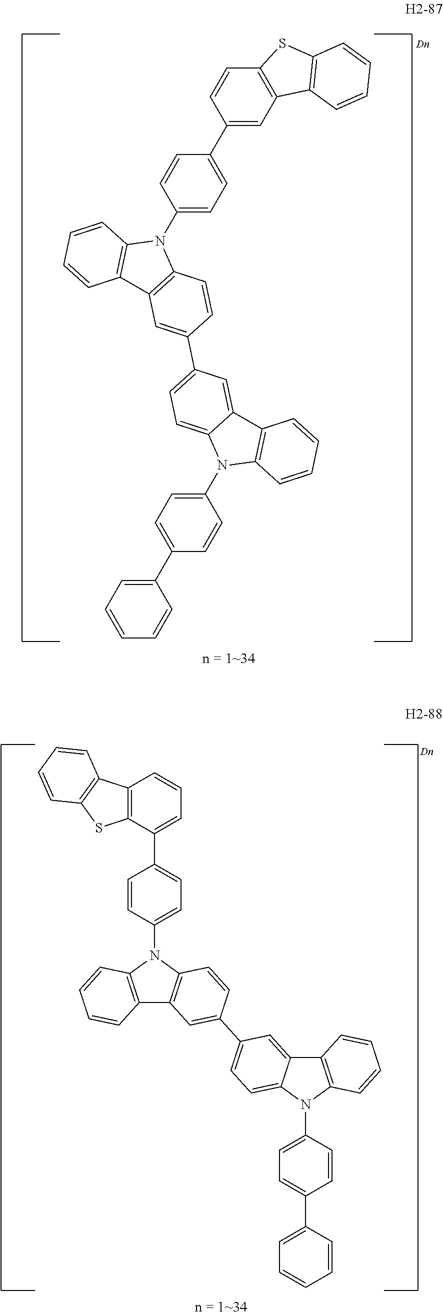 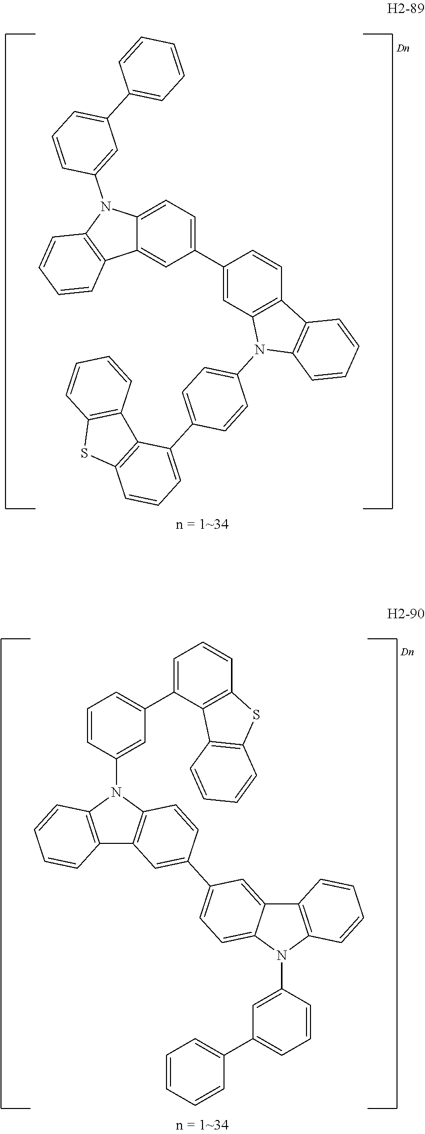 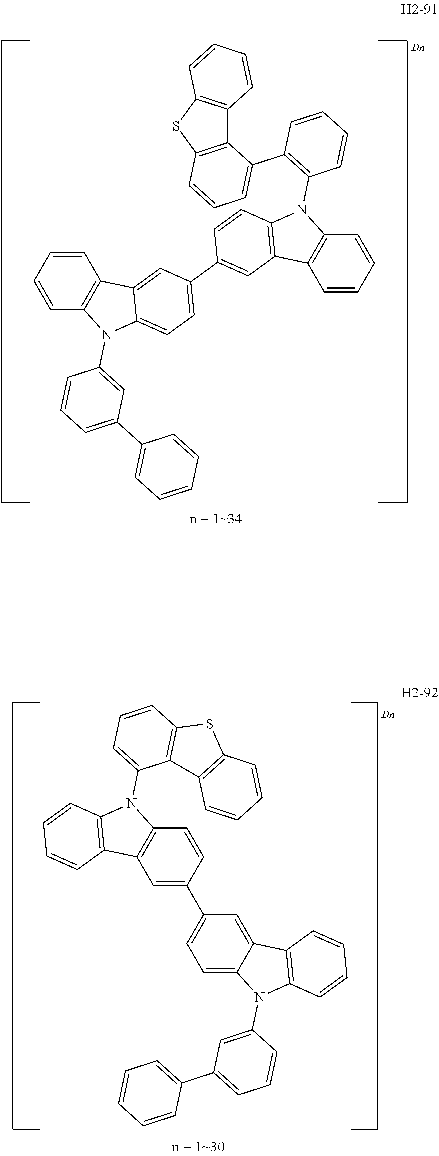 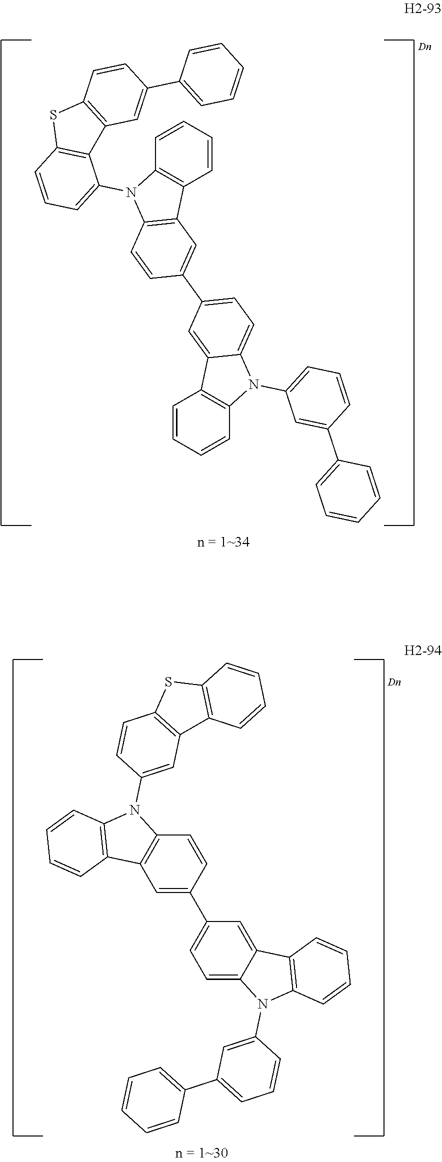 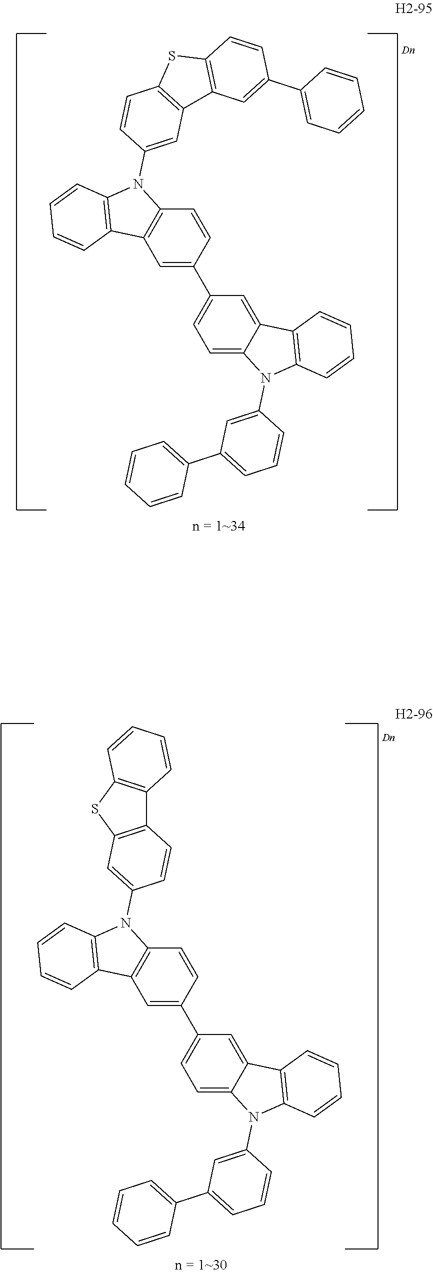 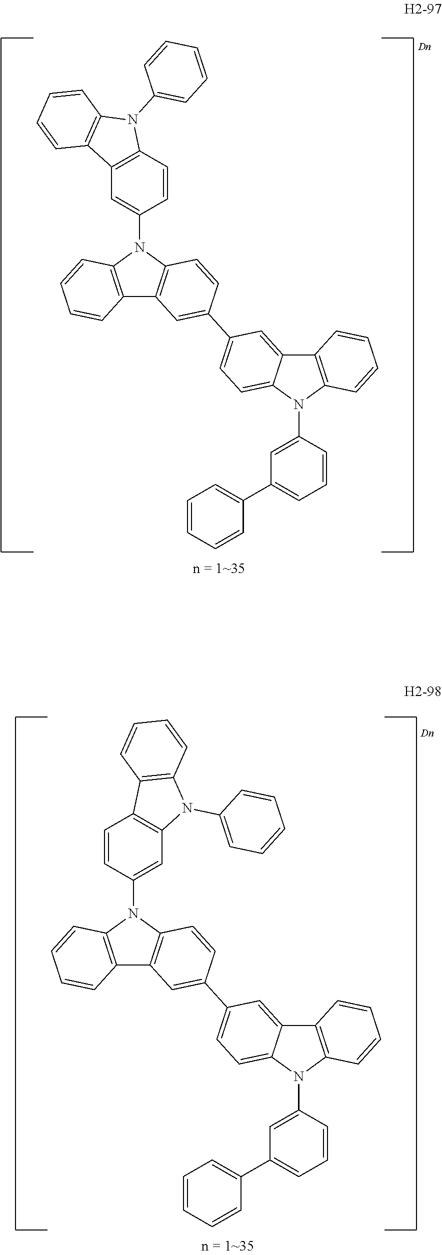 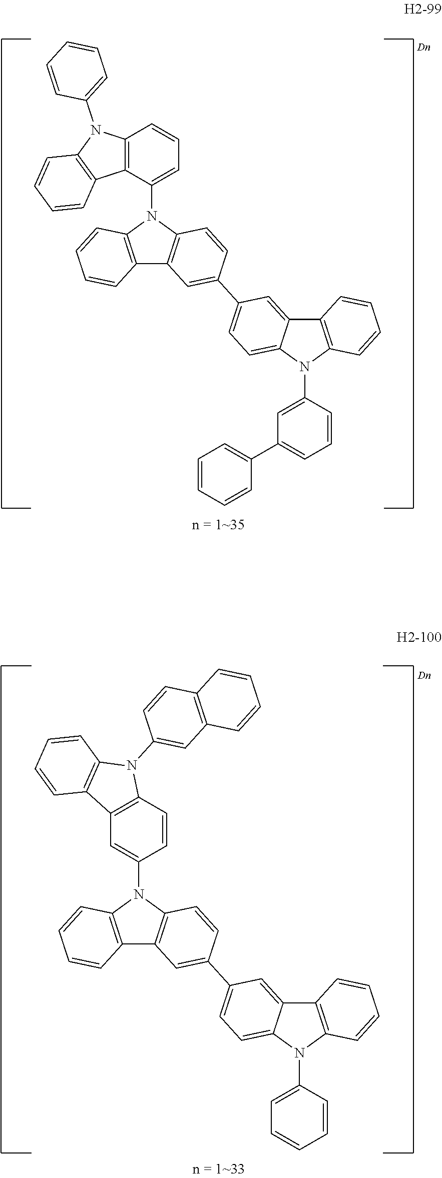 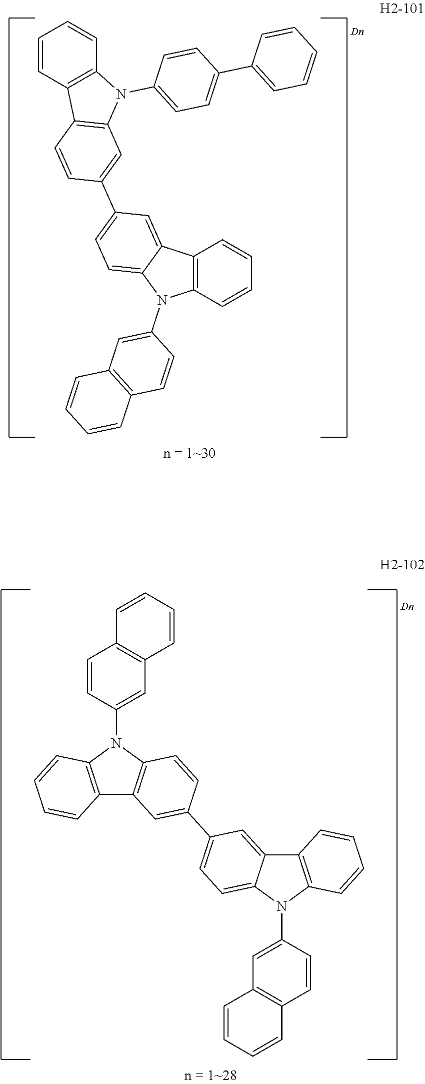 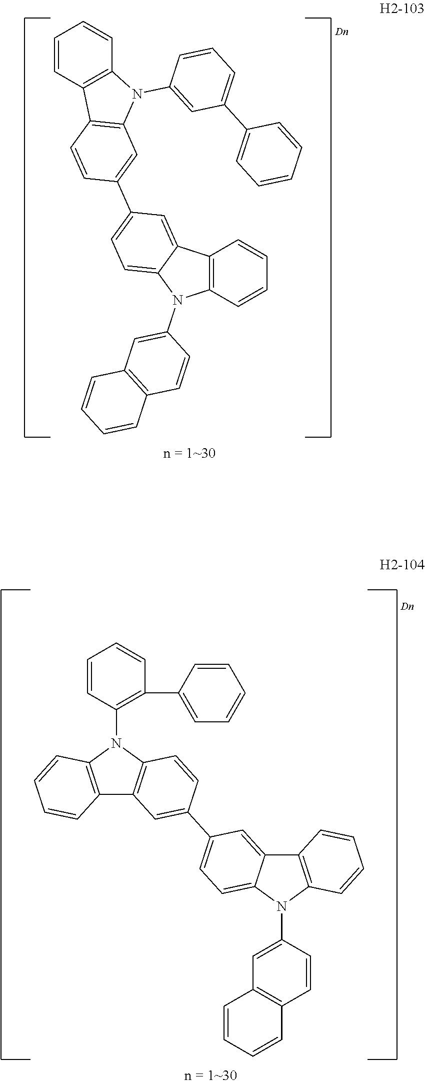 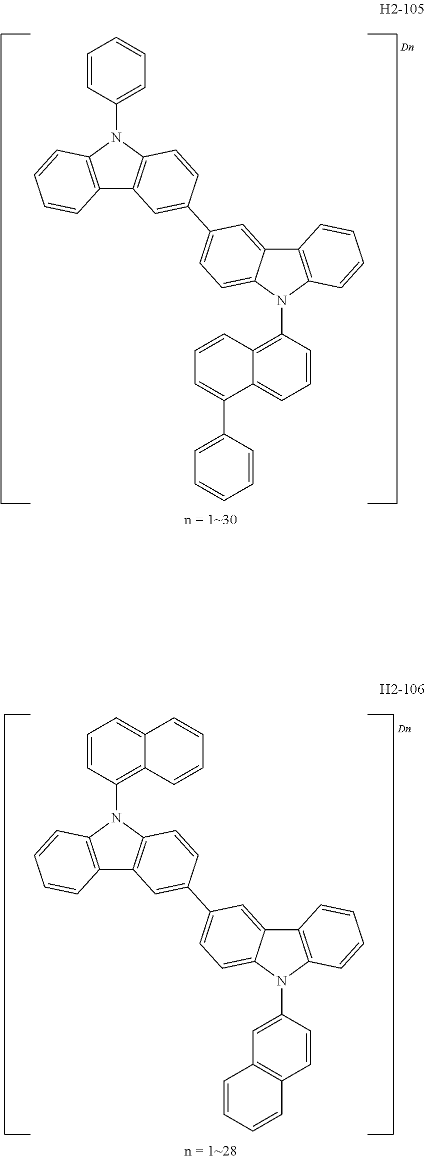 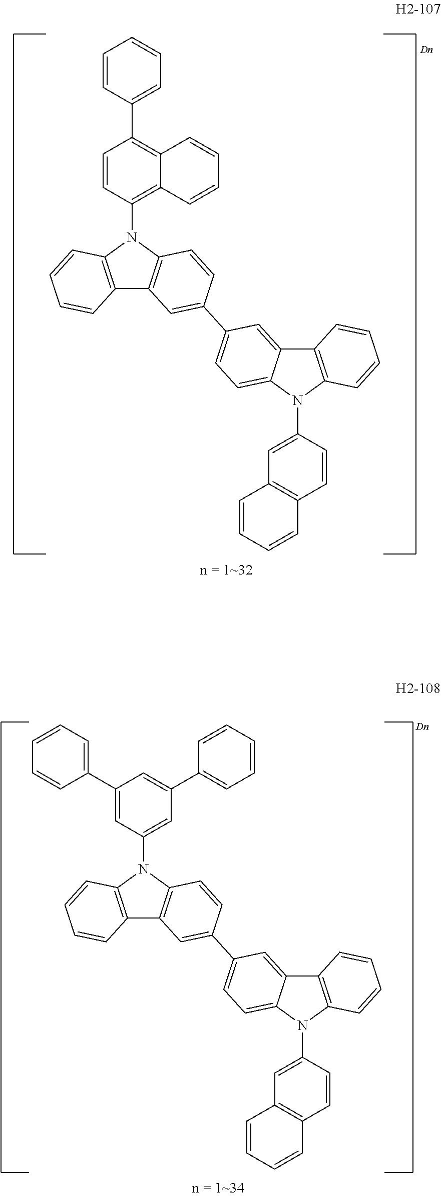 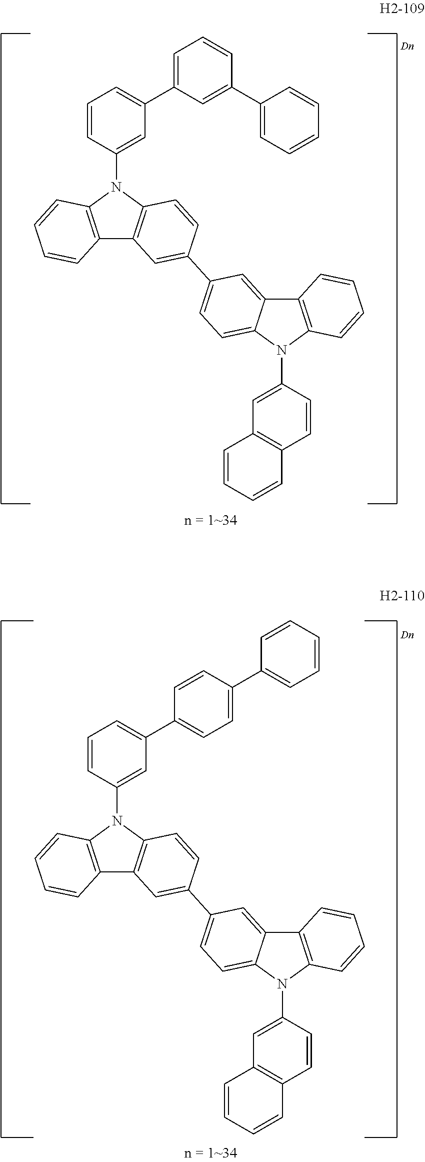 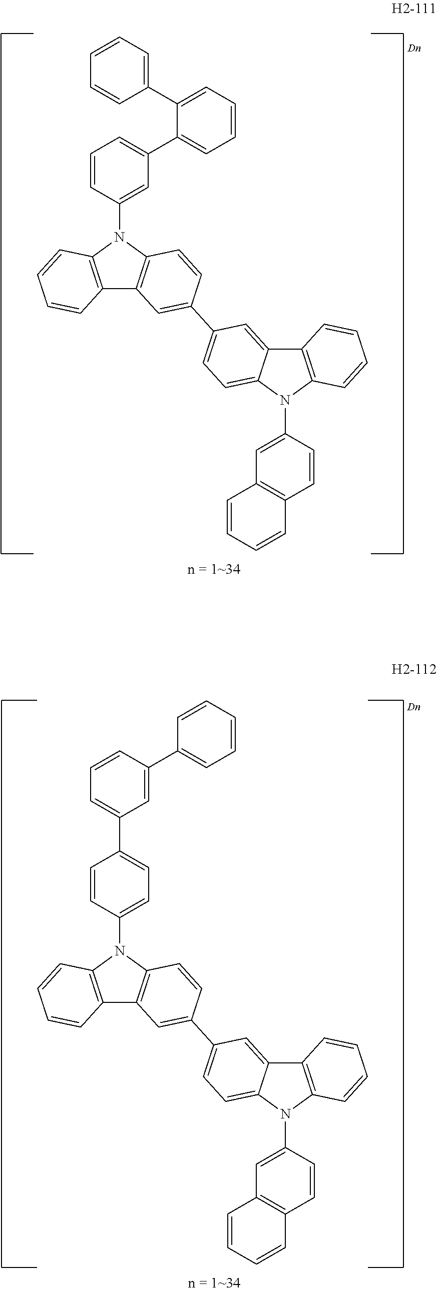 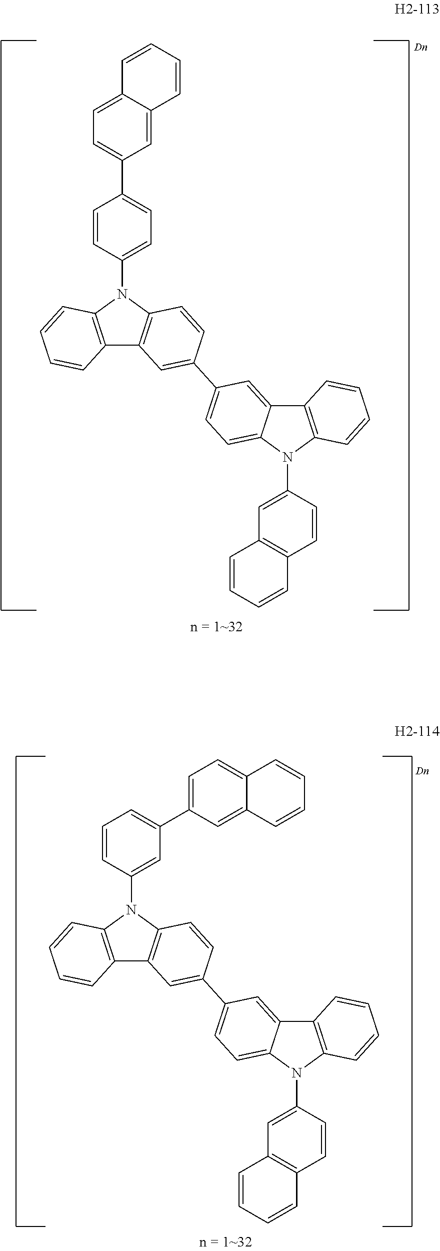 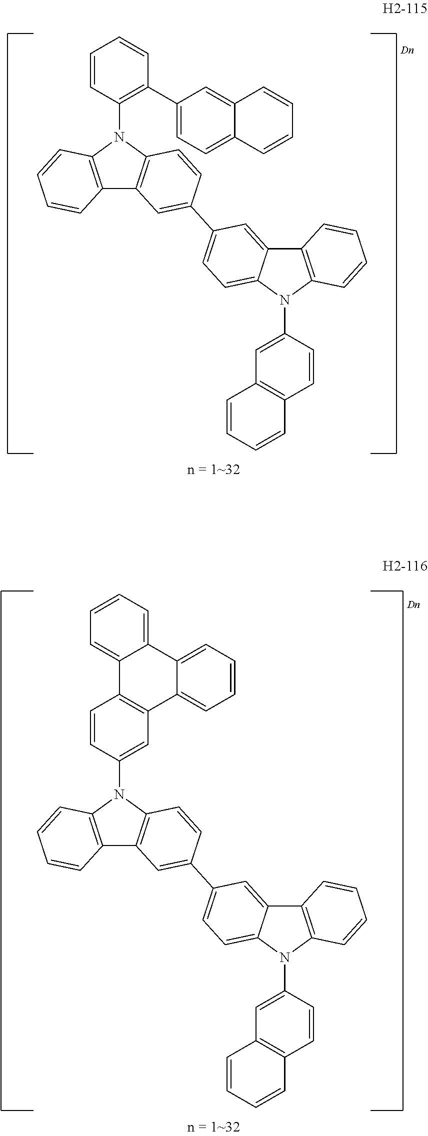 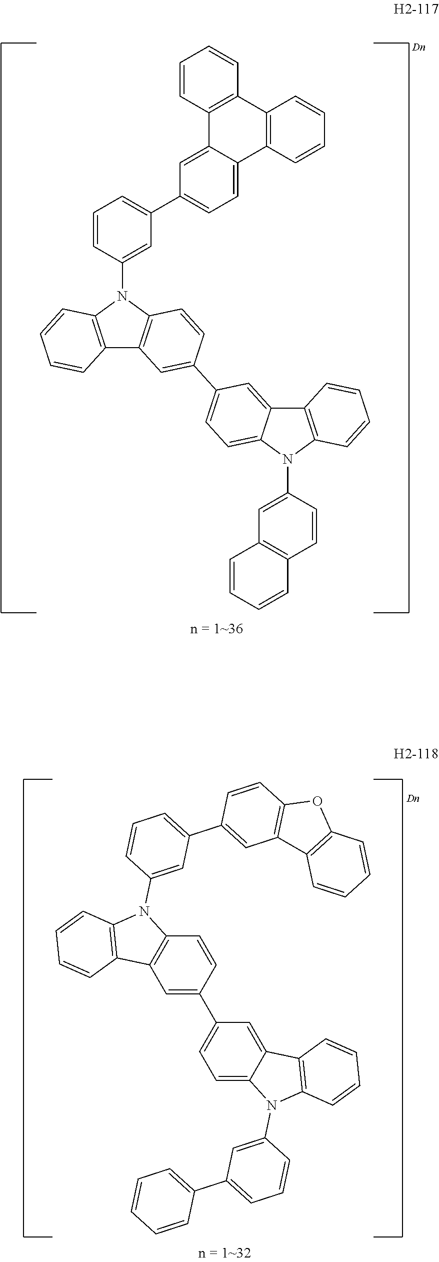 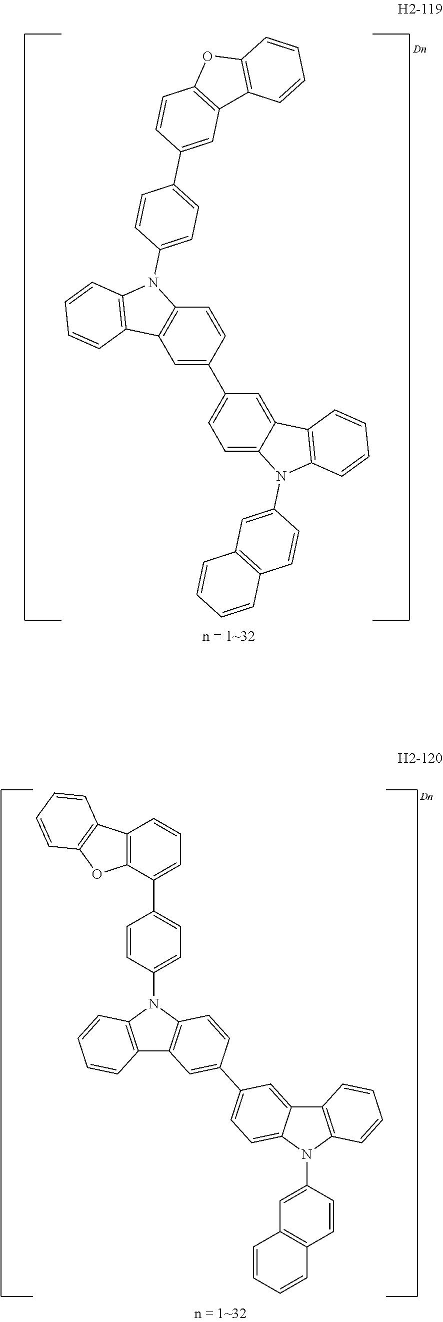 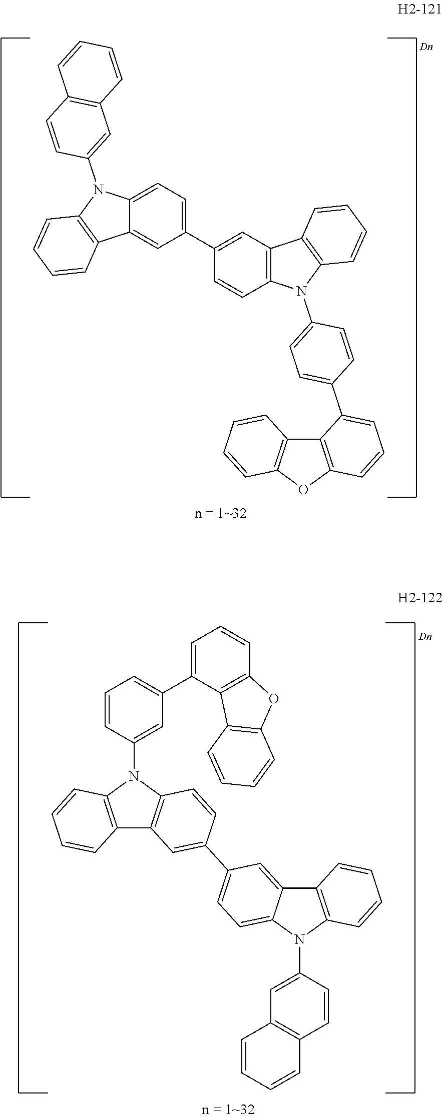 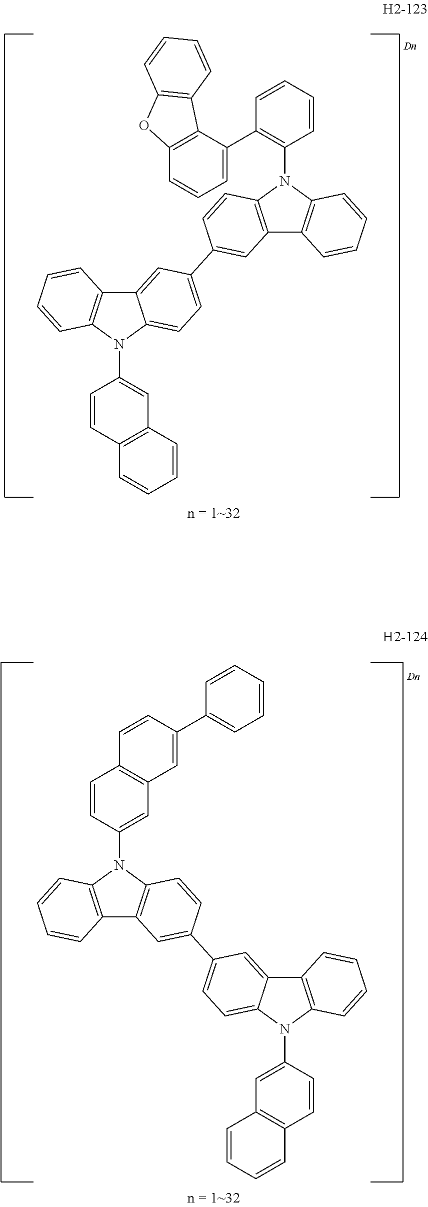 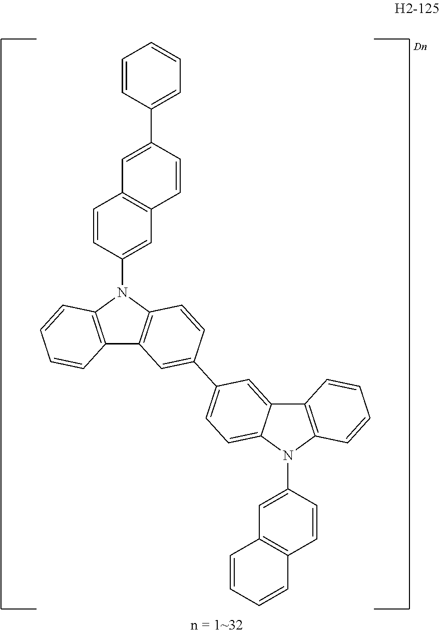 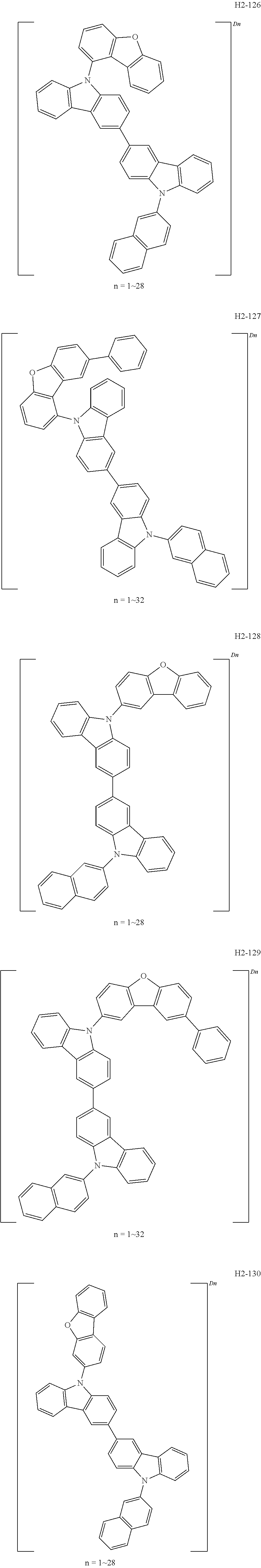 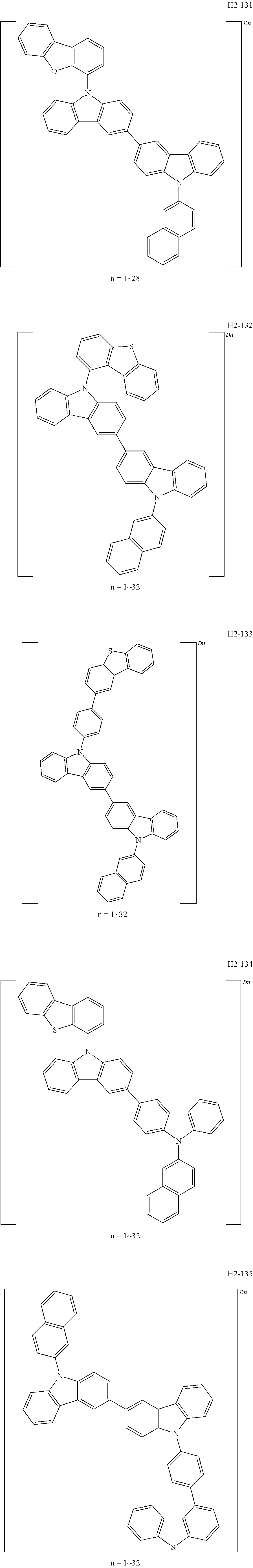 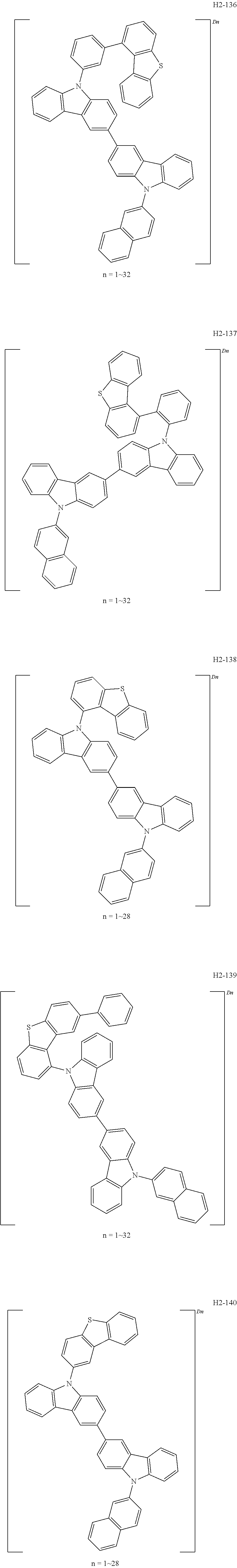 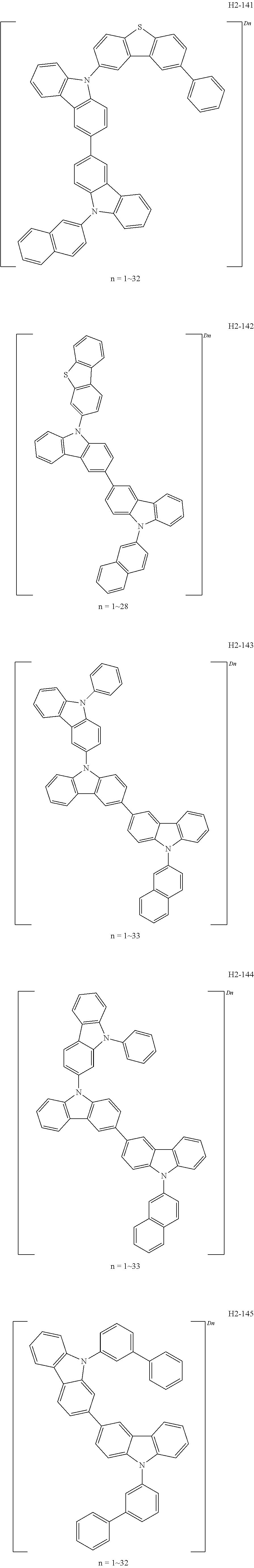 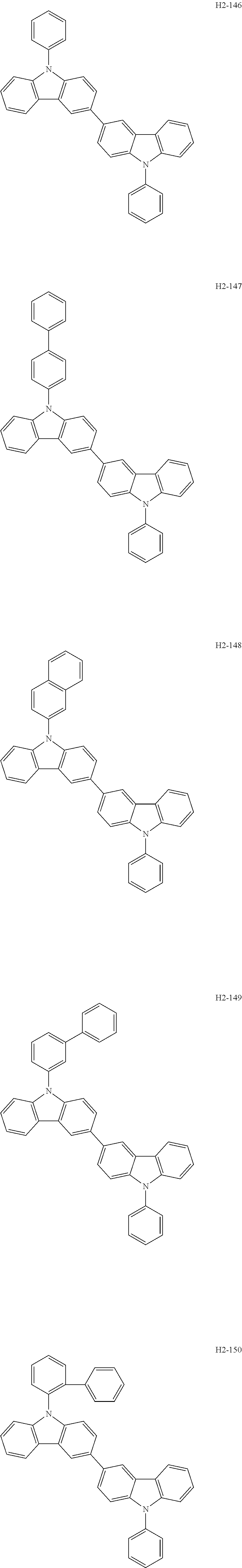 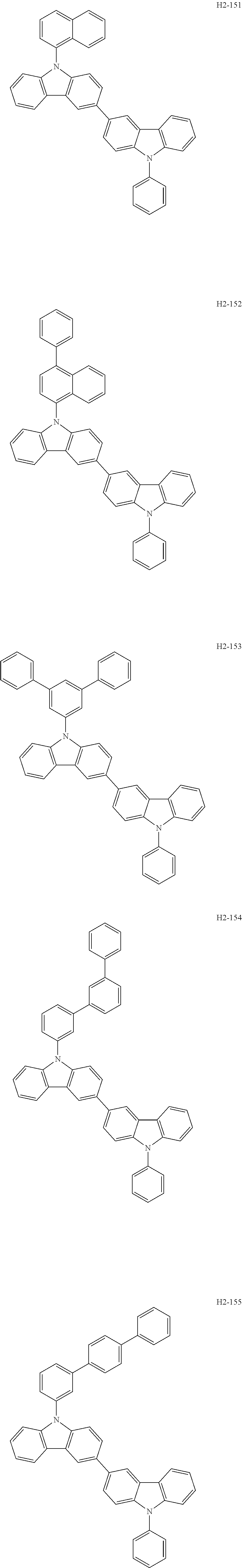 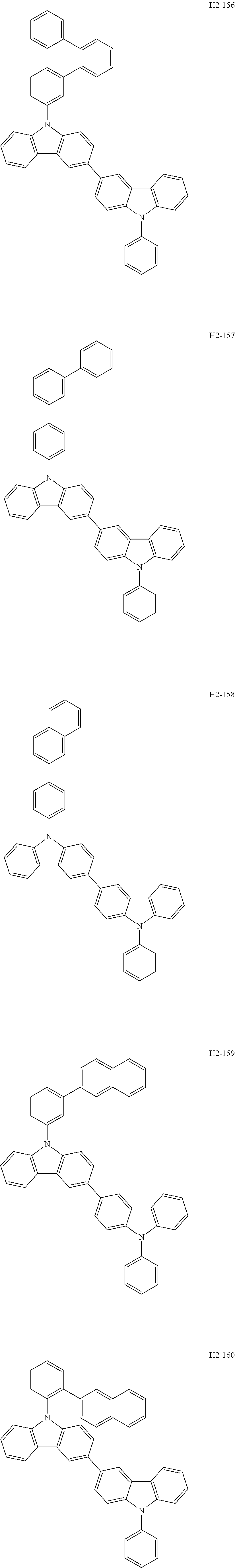 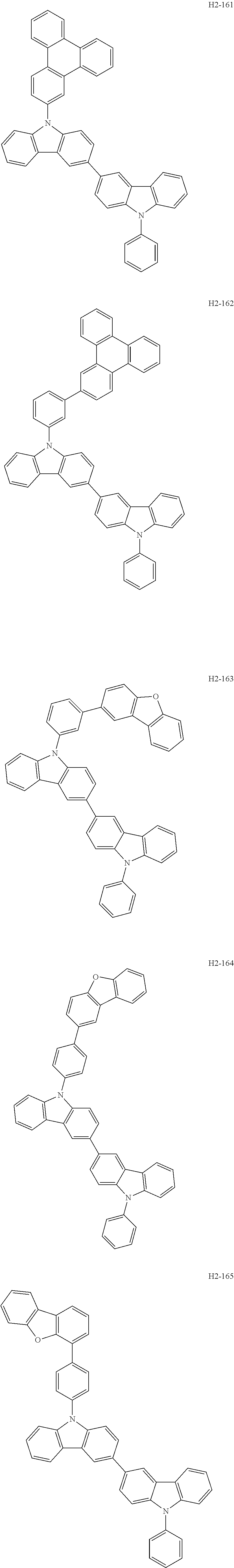 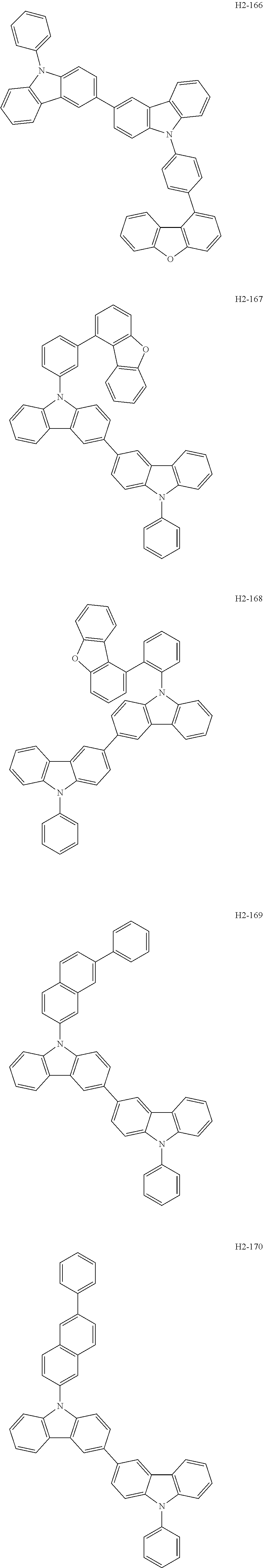 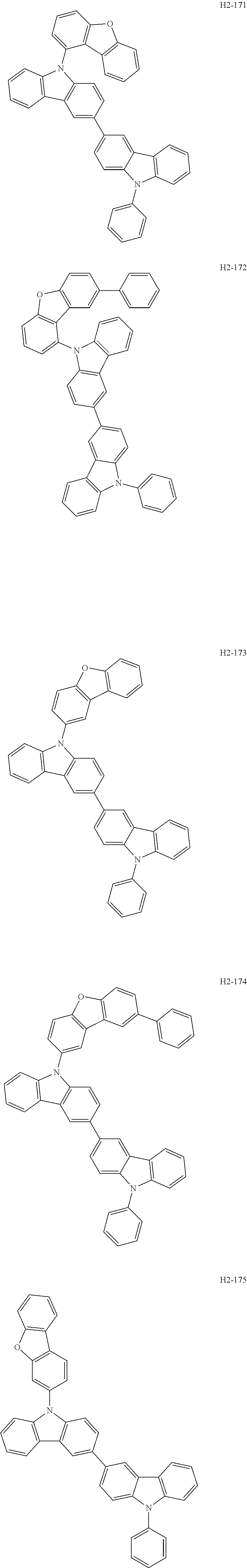 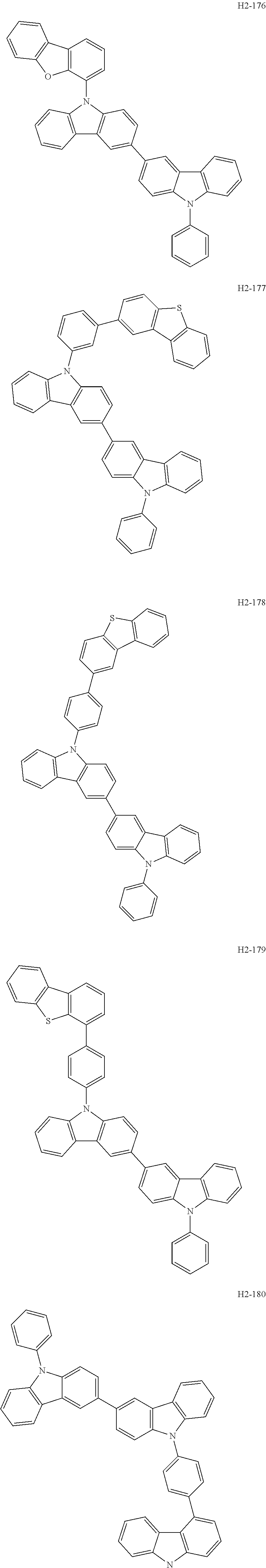 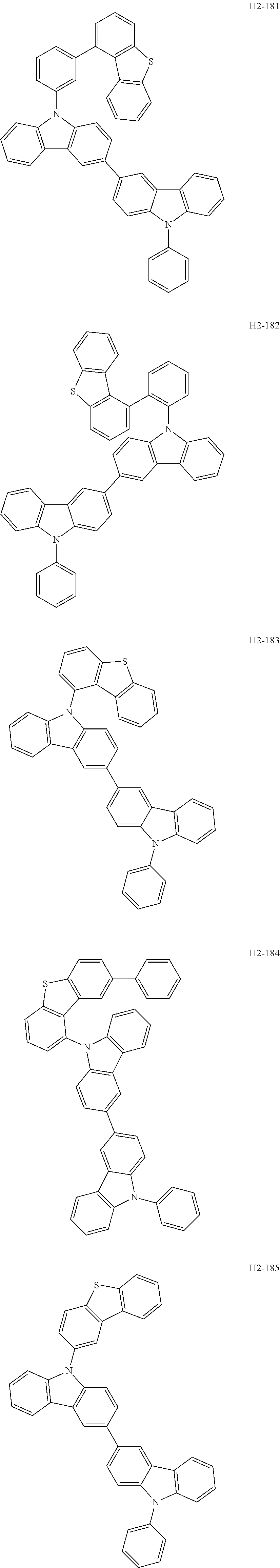 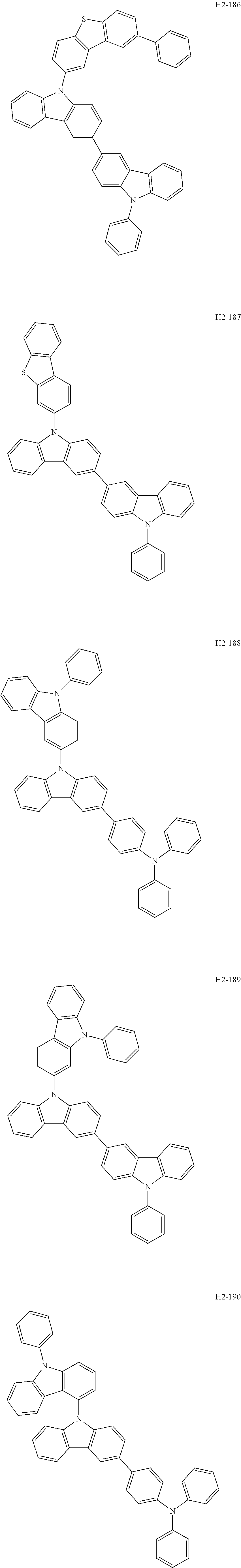 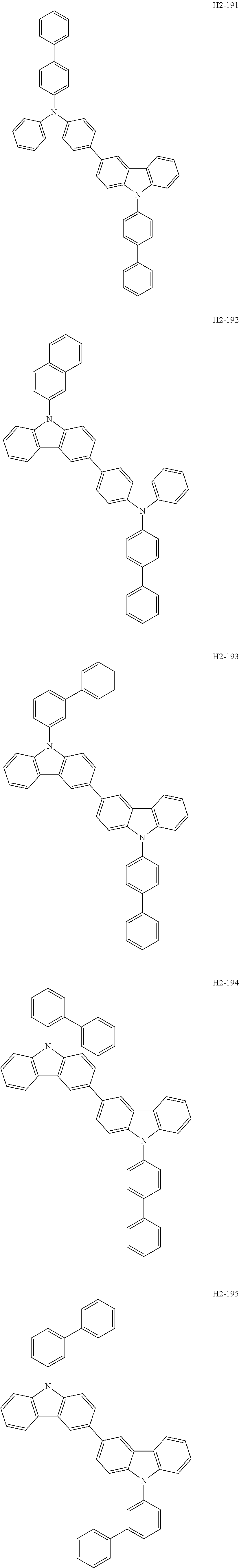 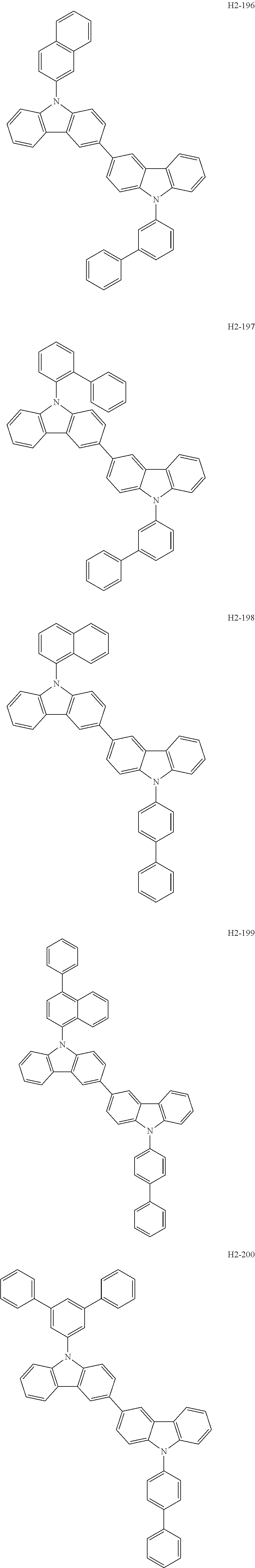 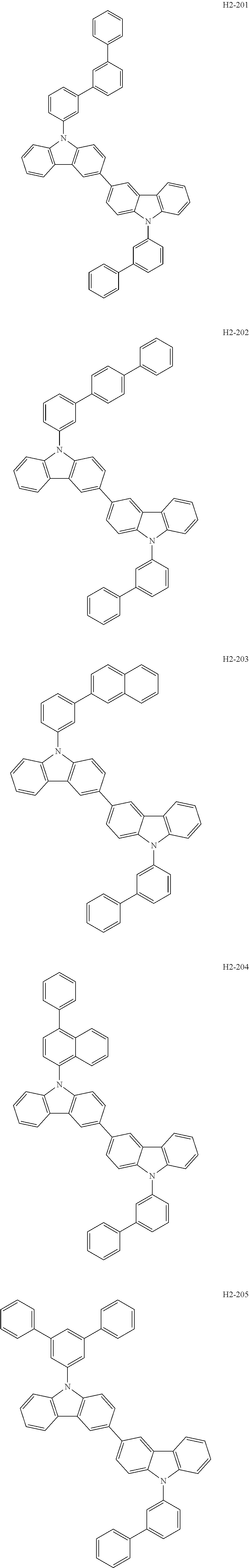 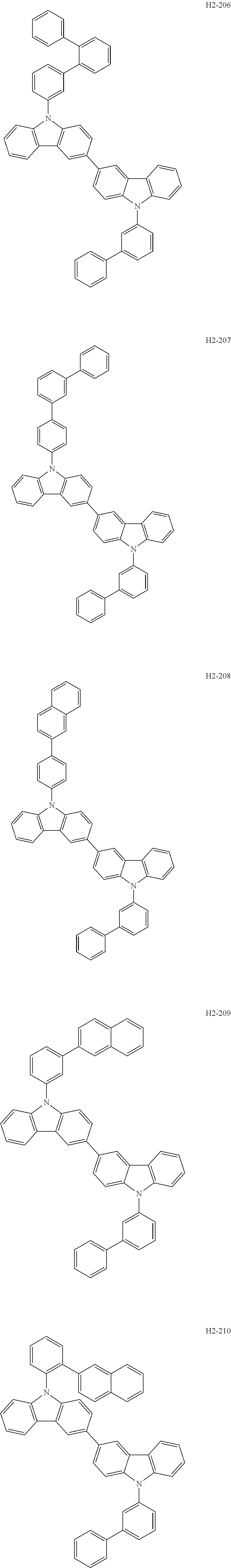 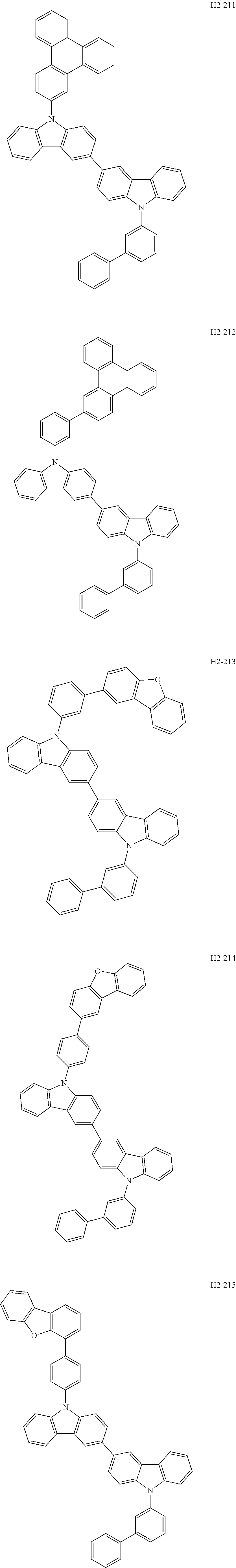 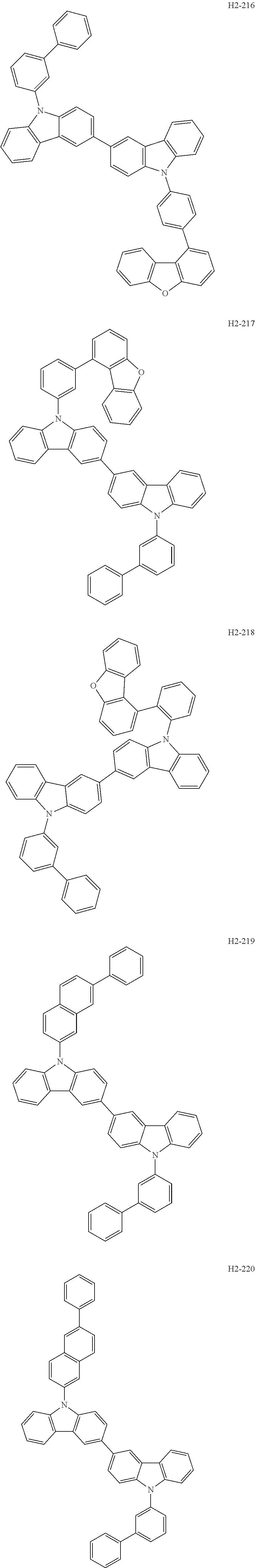 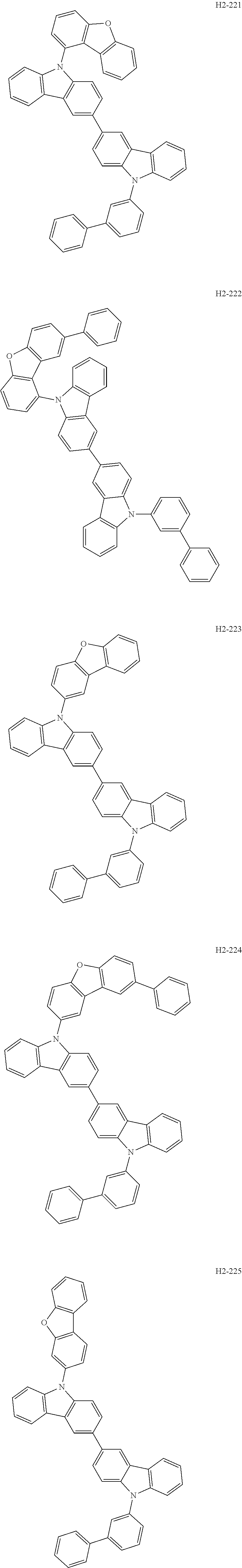 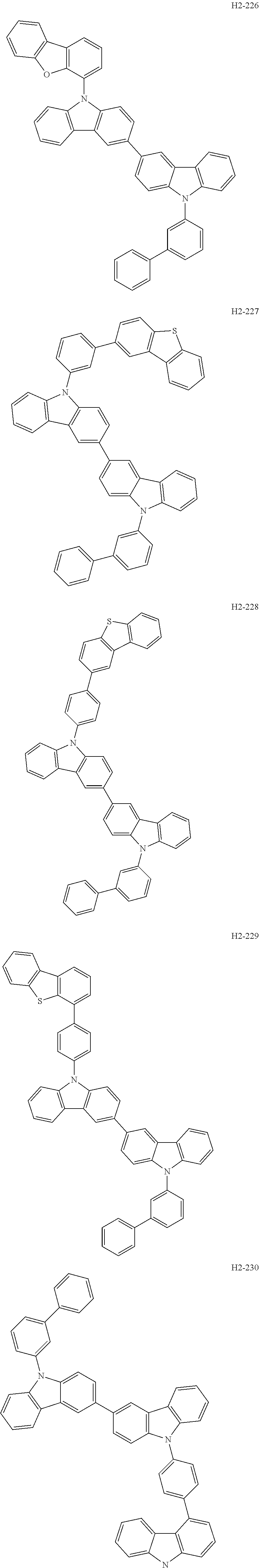 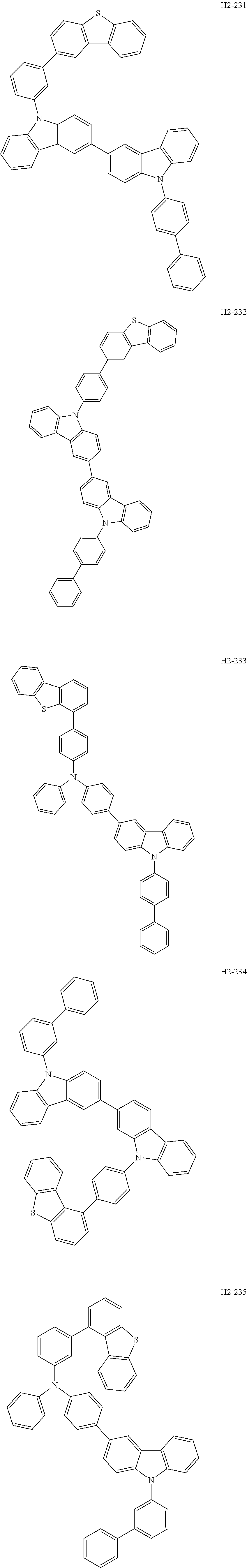 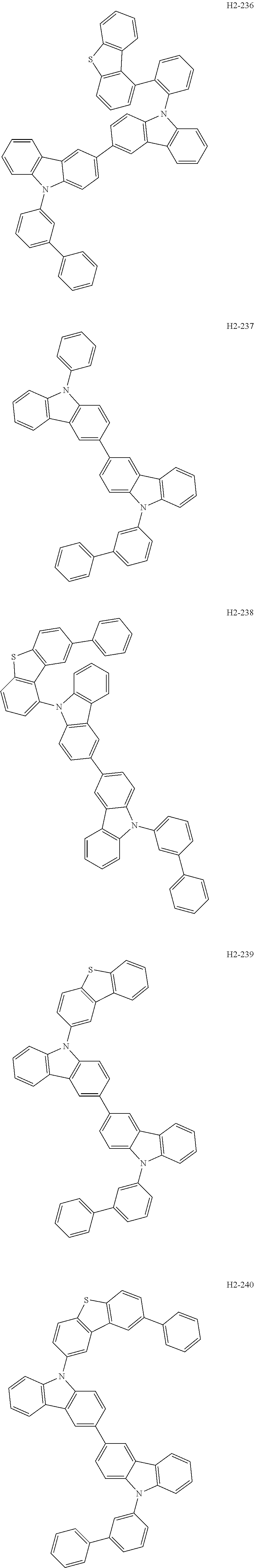 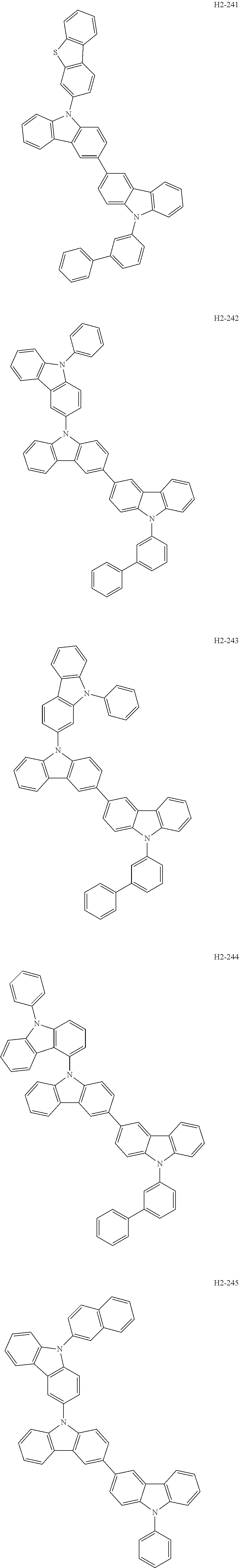 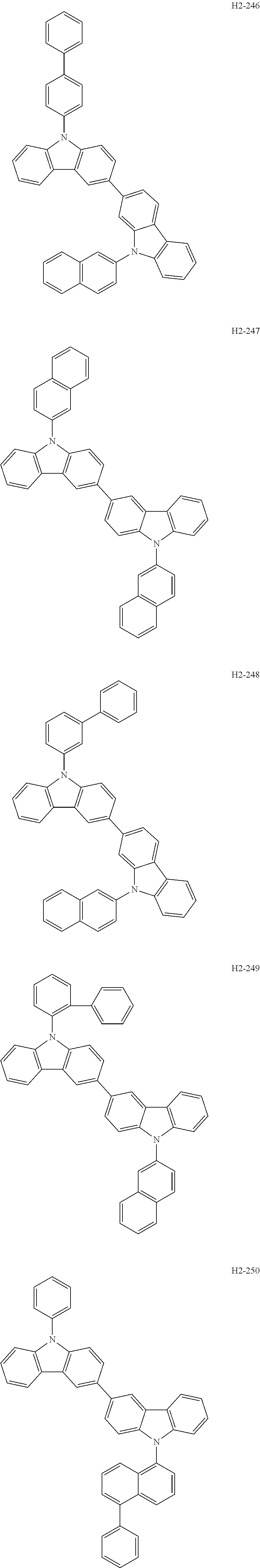 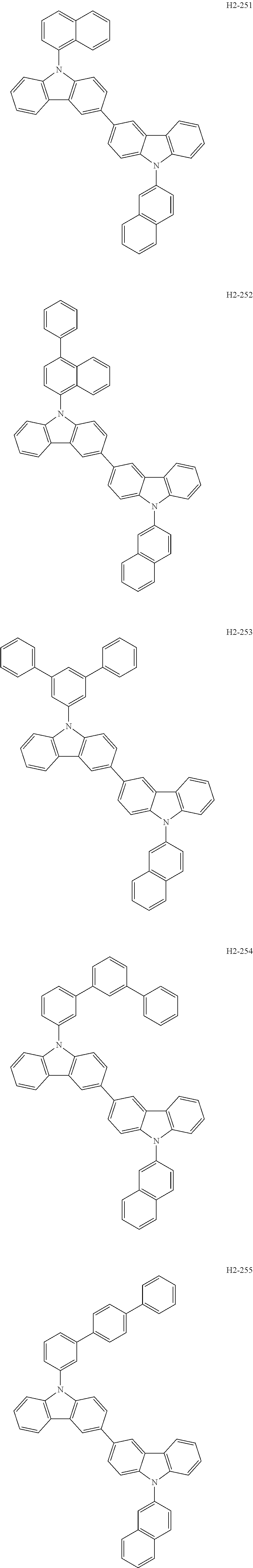 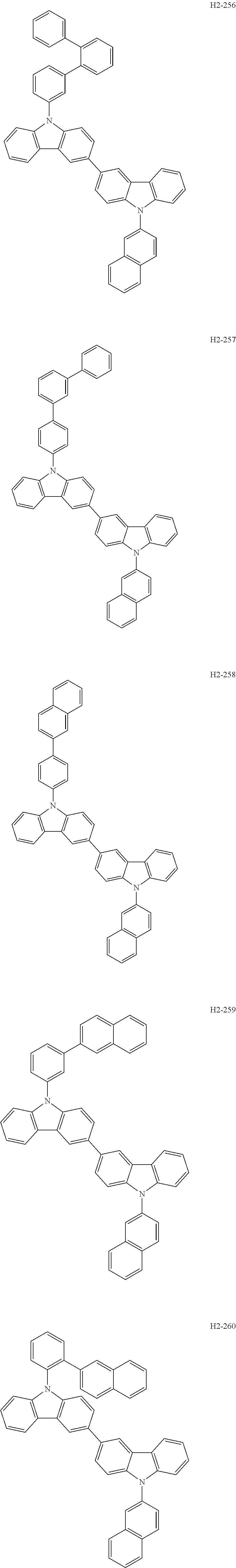 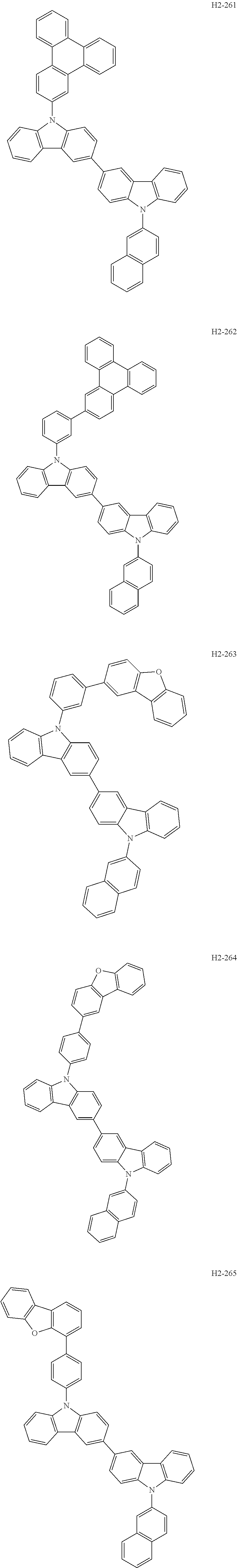 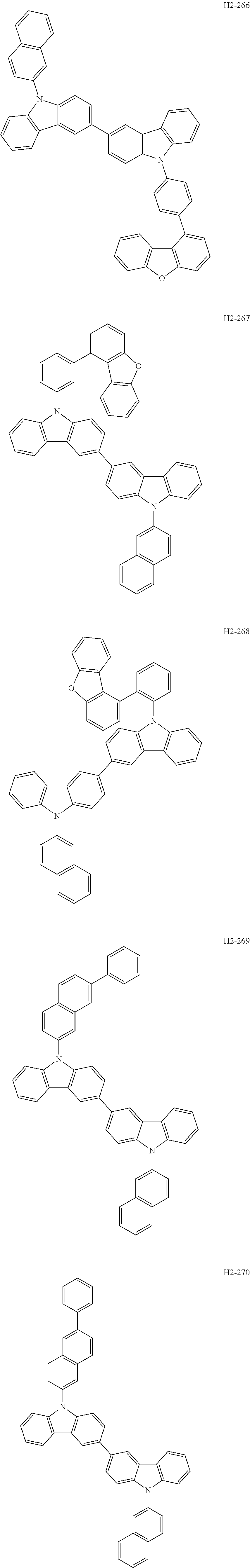 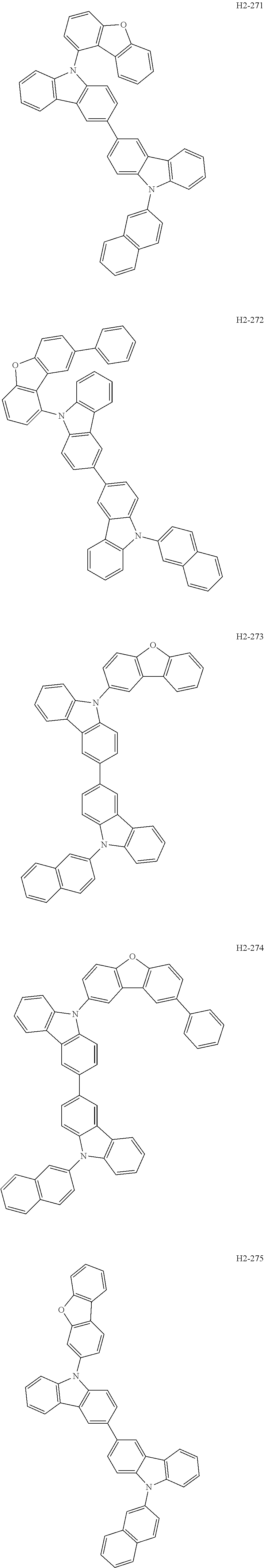 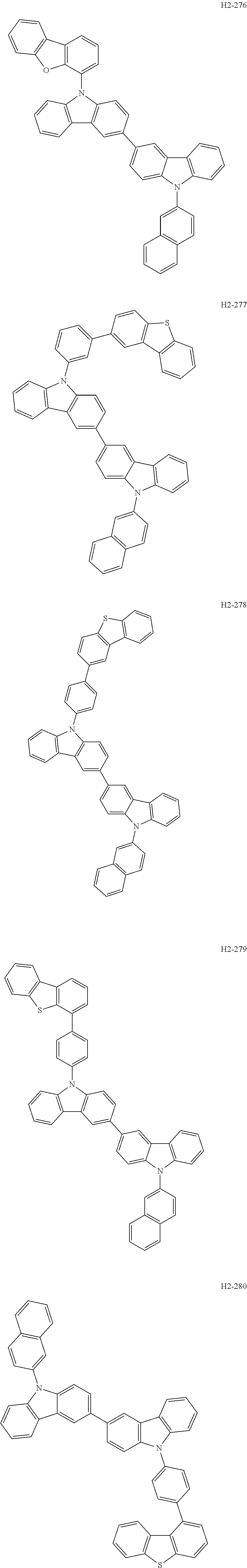 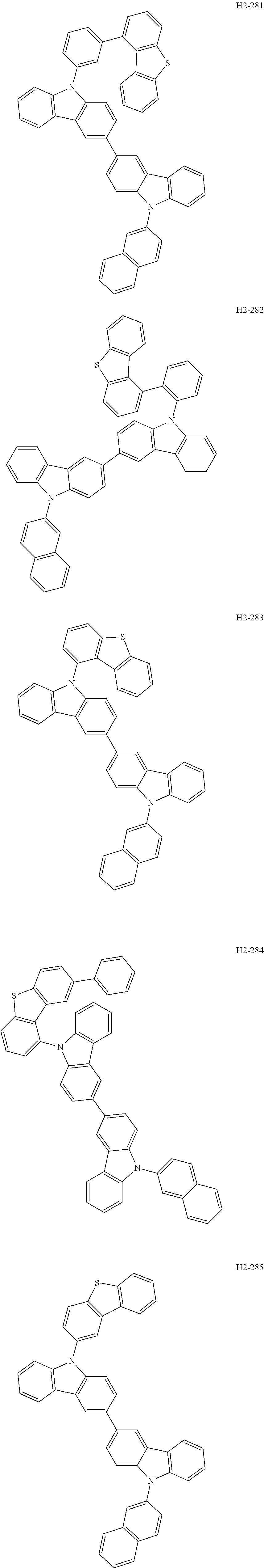 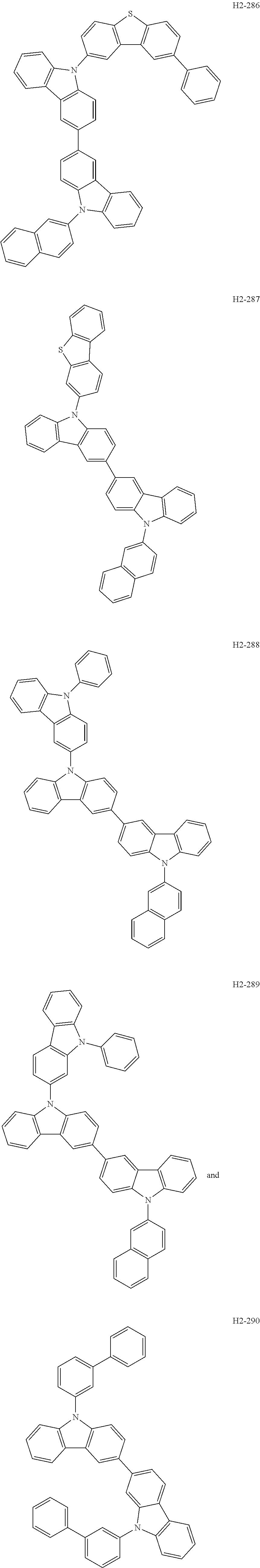
wherein Dn means that n number of hydrogen atoms are replaced with deuterium.

14. An organic electroluminescent device comprising an anode; a cathode; and at least one light-emitting layer between the anode and the cathode, wherein the at least one light-emitting layer comprises the plurality of host materials according to claim 1.

15. An organic electroluminescent compound represented by the following formula 1-1: 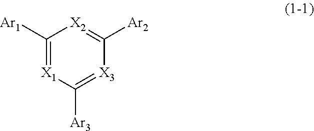
in formula 1-1,
X₁ to X₃ each independently represent N or CRₐ; with the proviso that at least two of X₁ to X₃ are N;
Rₐ represents hydrogen or deuterium; and
Ar₁ to Ar₃ each independently represent a (C6-C30)aryl unsubstituted or substituted with at least one of deuterium and a (C6-C30)aryl(s); with the proviso that Ar₁ to Ar₃ are different from each other, and the structure comprising naphthalene and the following structures are excluded:

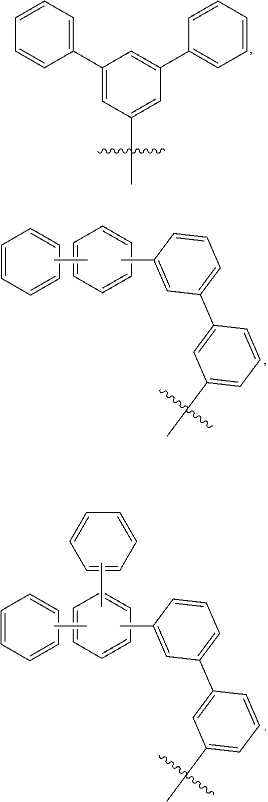

16. The organic electroluminescent compound according to claim 15, wherein Ar₁ to Ar₃ of formula 1-1 are each independently a phenyl unsubstituted or substituted with deuterium, a biphenyl unsubstituted or substituted with deuterium, a terphenyl unsubstituted or substituted with deuterium, a quaterphenyl unsubstituted or substituted with deuterium, a phenylnaphthyl unsubstituted or substituted with deuterium, a naphthylphenyl unsubstituted or substituted with deuterium, a triphenylenyl unsubstituted or substituted with deuterium, a phenanthrenyl unsubstituted or substituted with deuterium, or a combination thereof.

17. The organic electroluminescent compound according to claim 15, wherein the compound represented by formula 1-1 is selected from the following compounds: 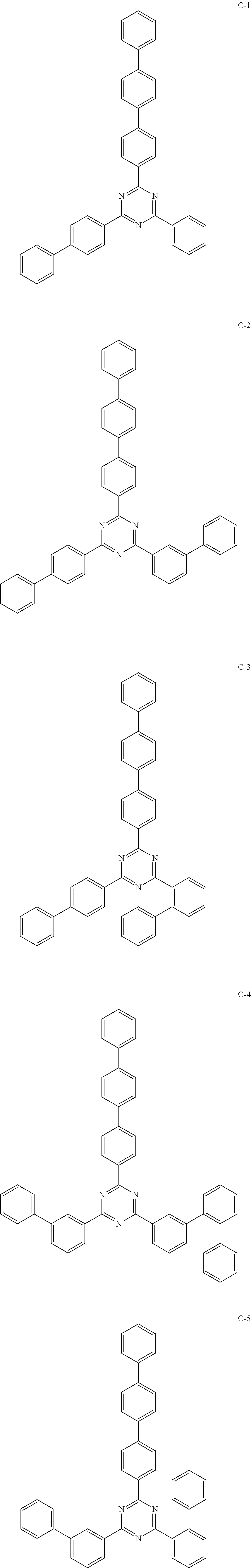 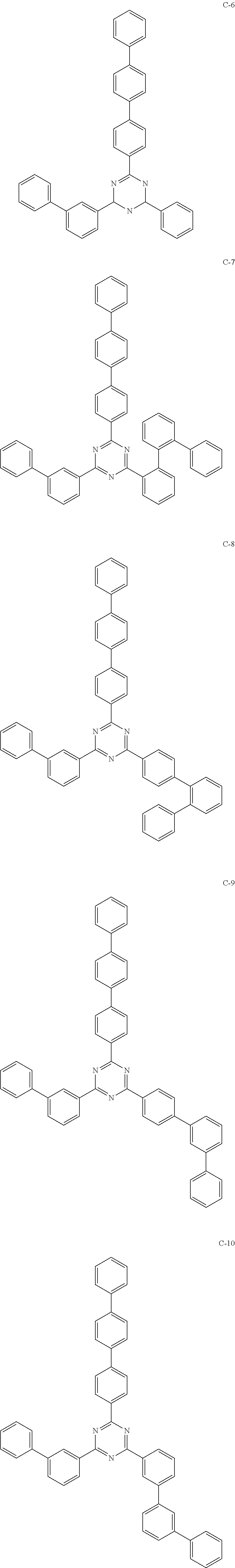 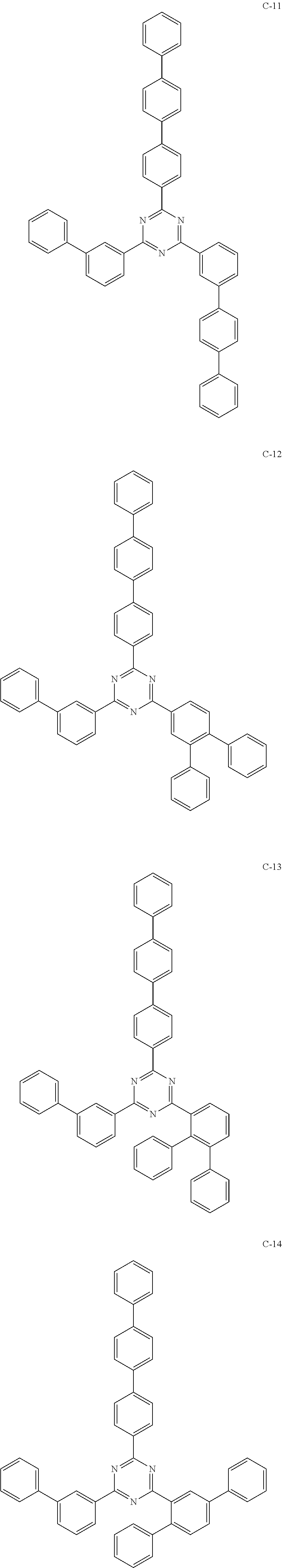 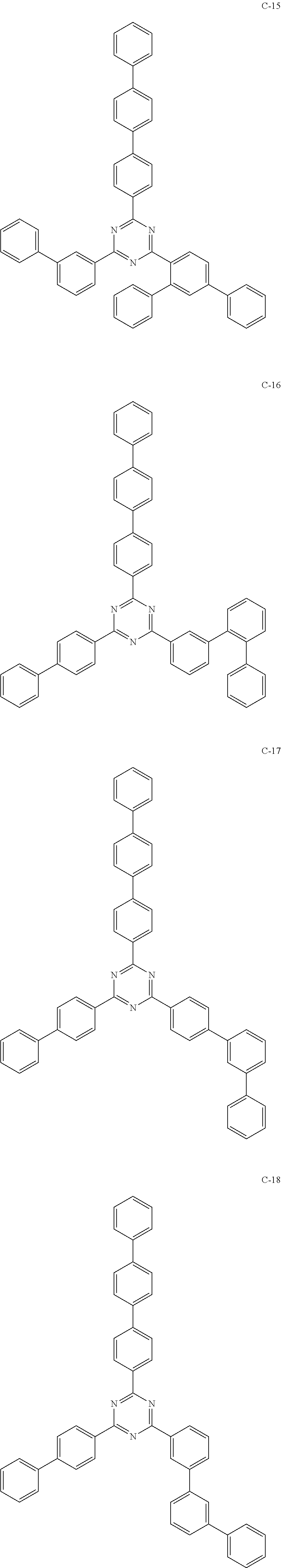 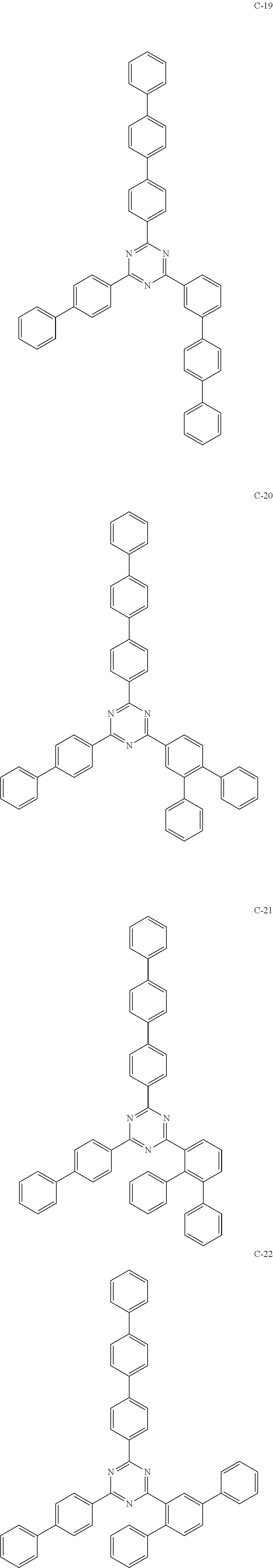 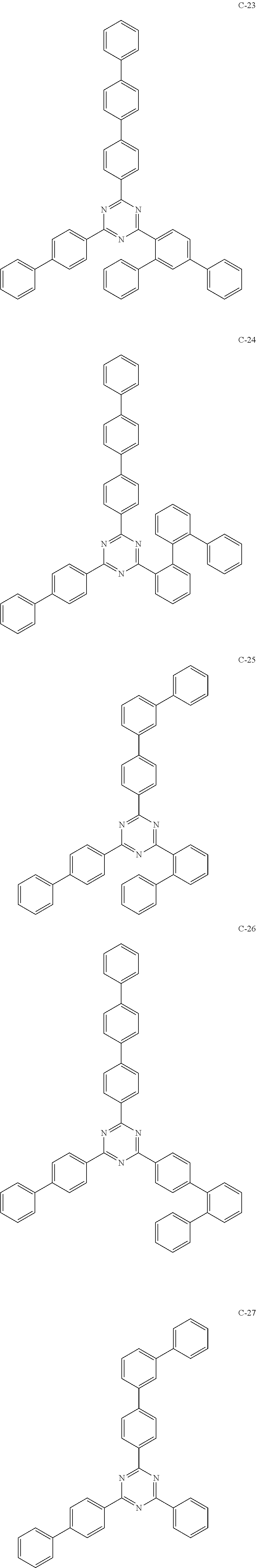 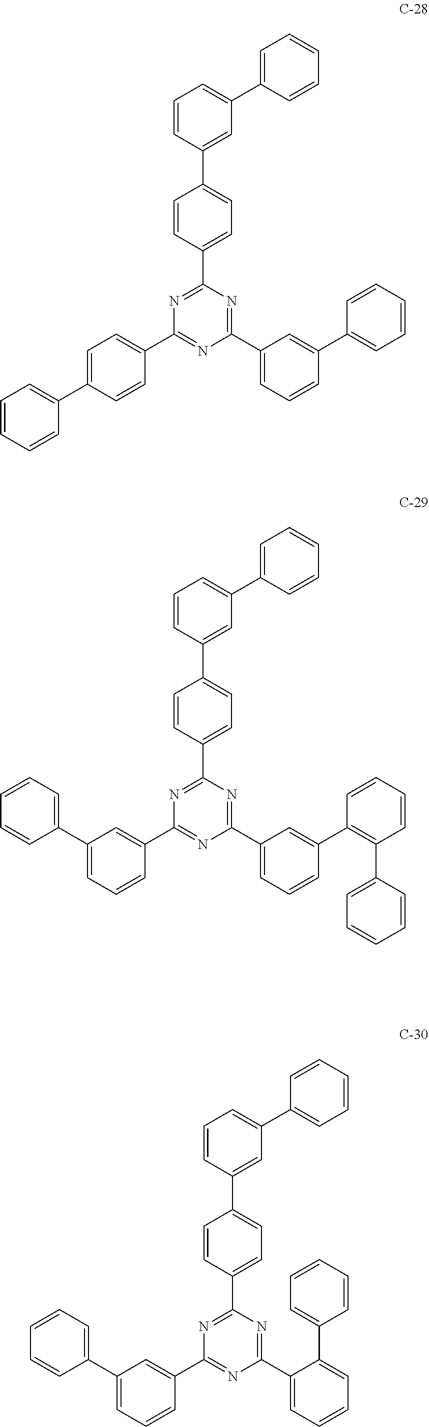 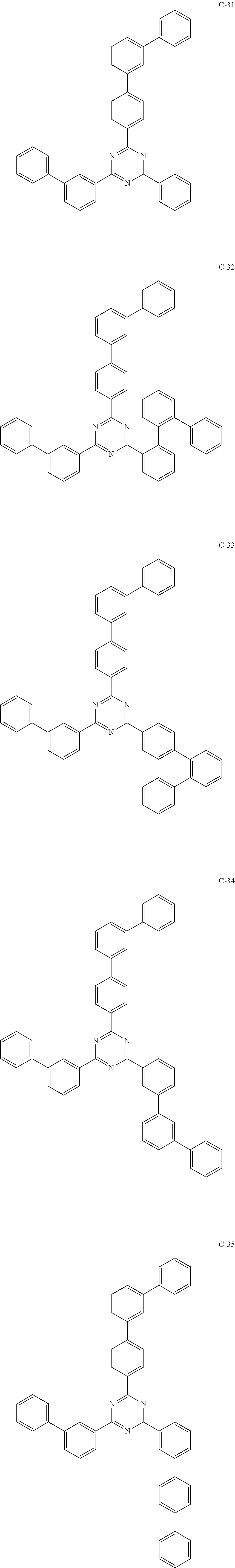 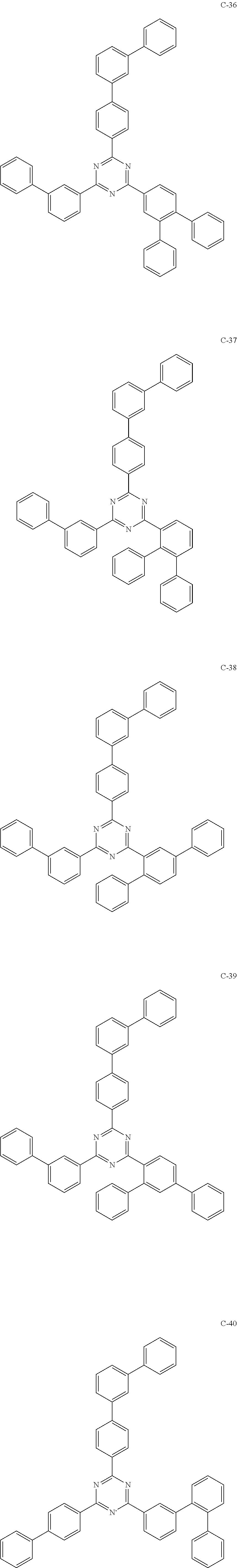 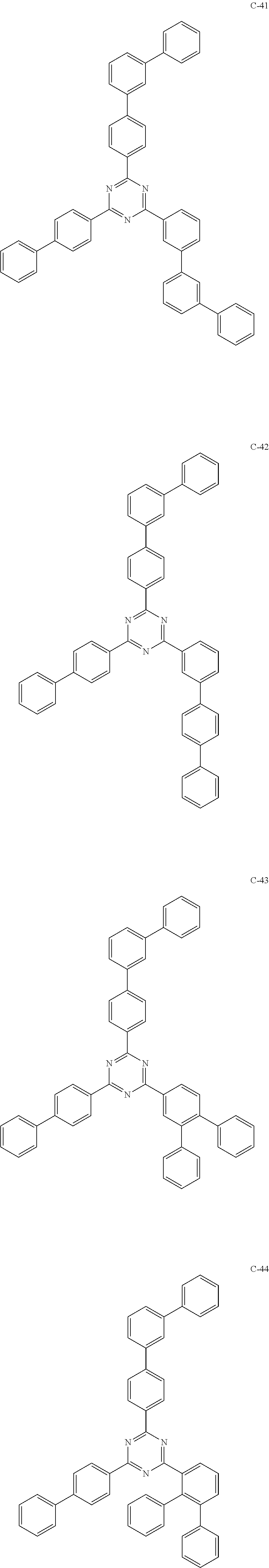 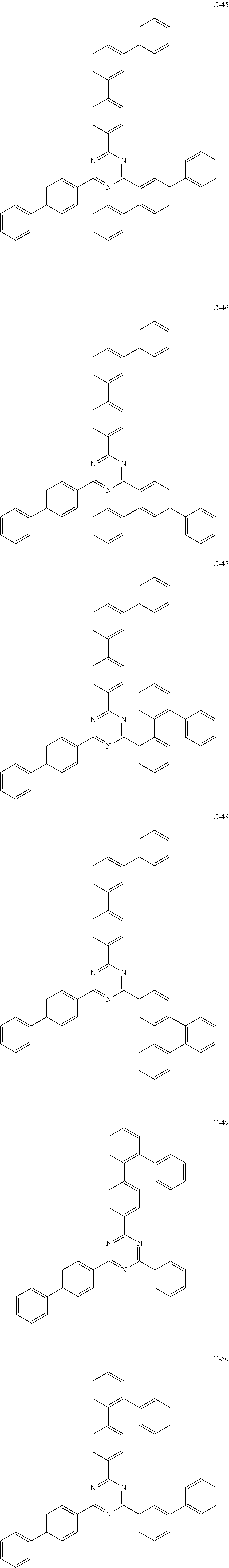 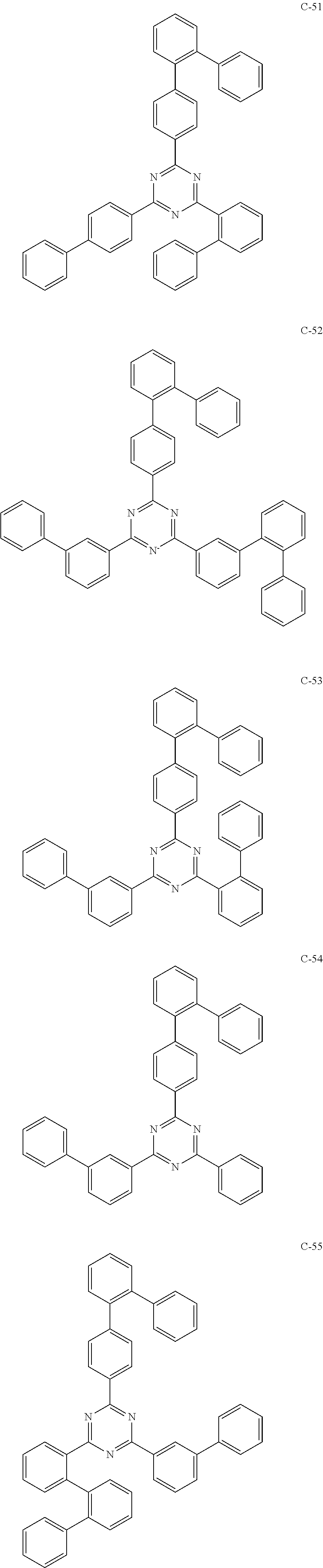 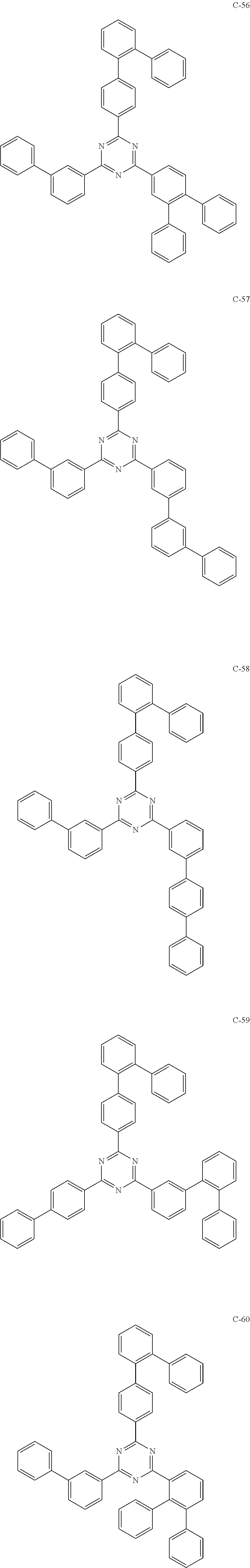 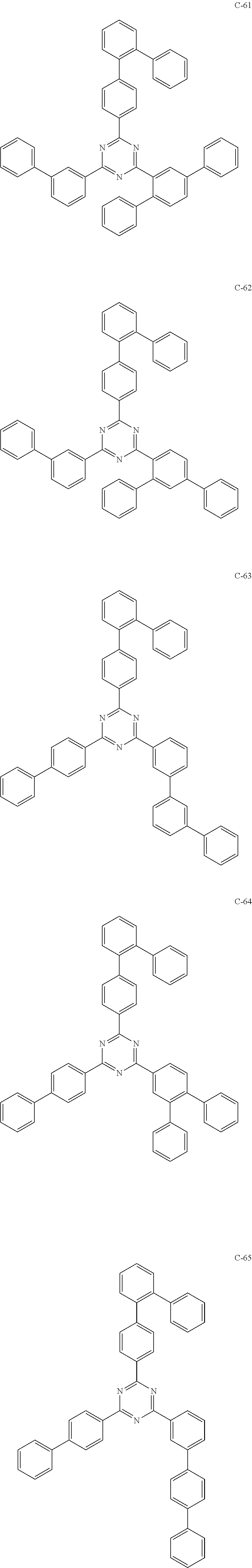 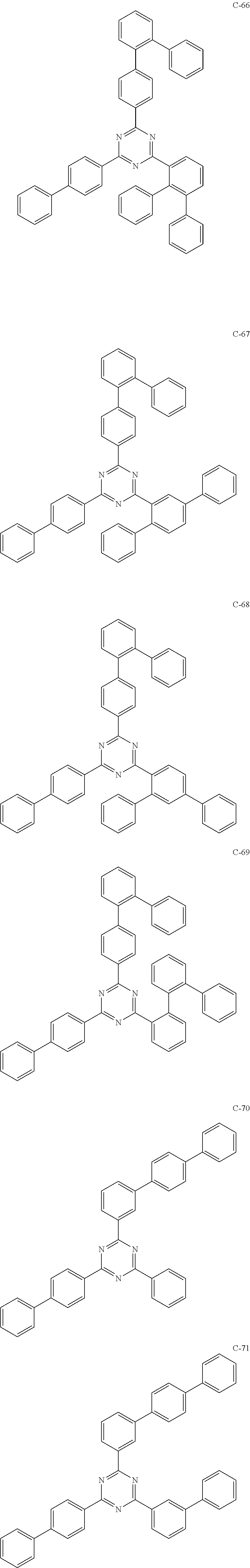 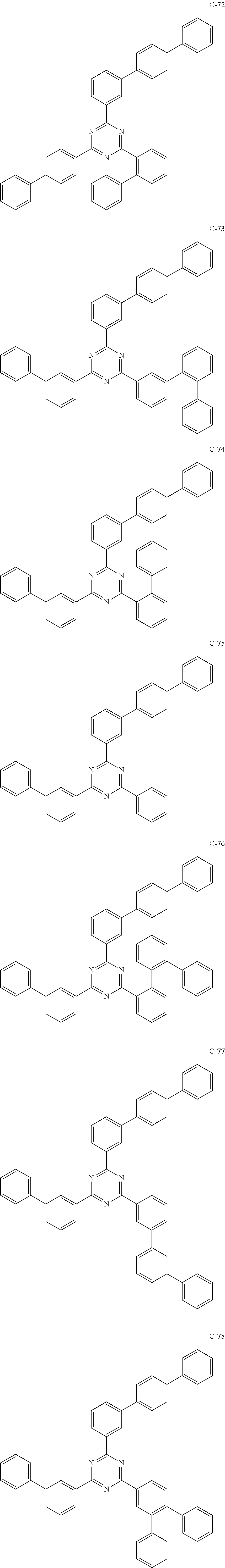 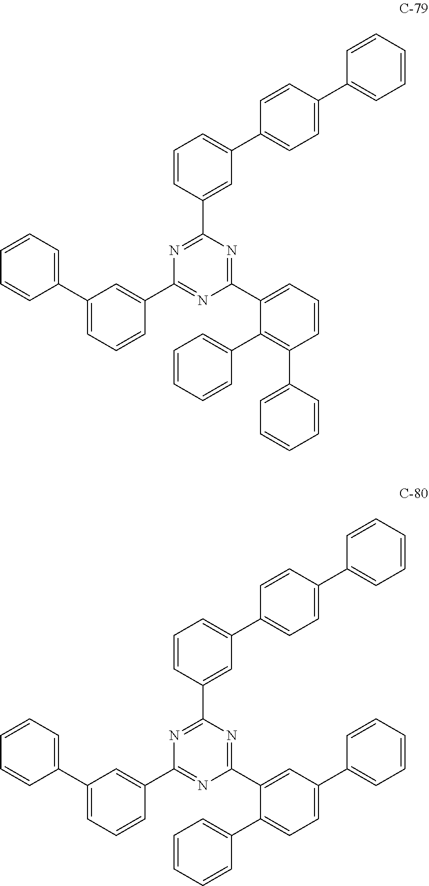 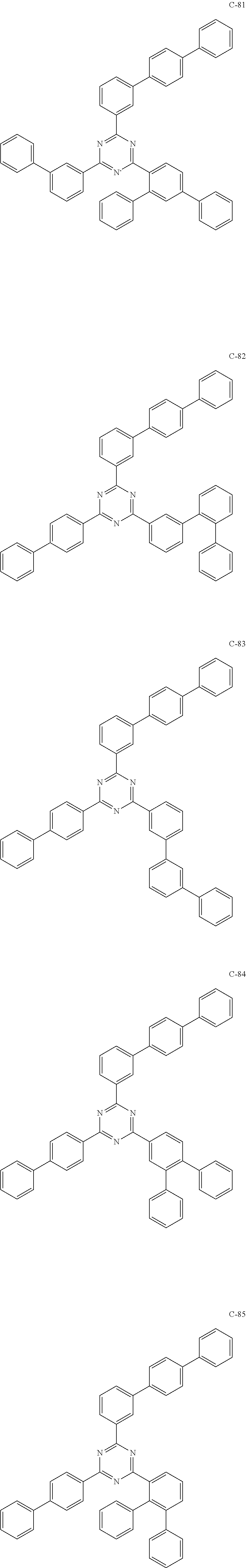 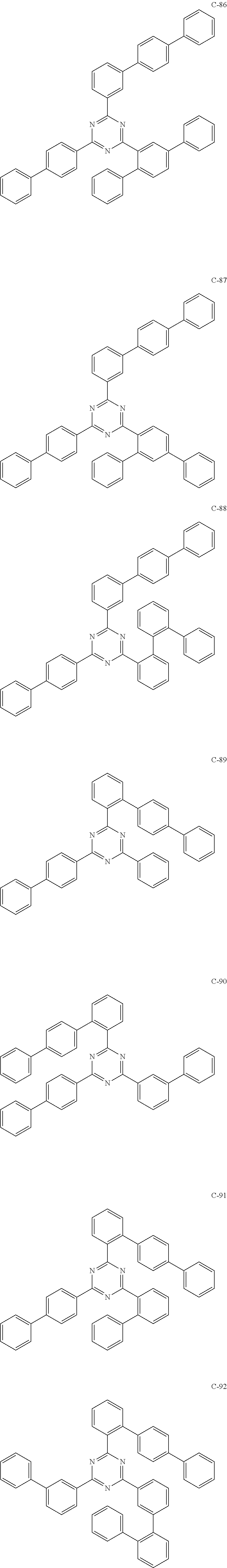 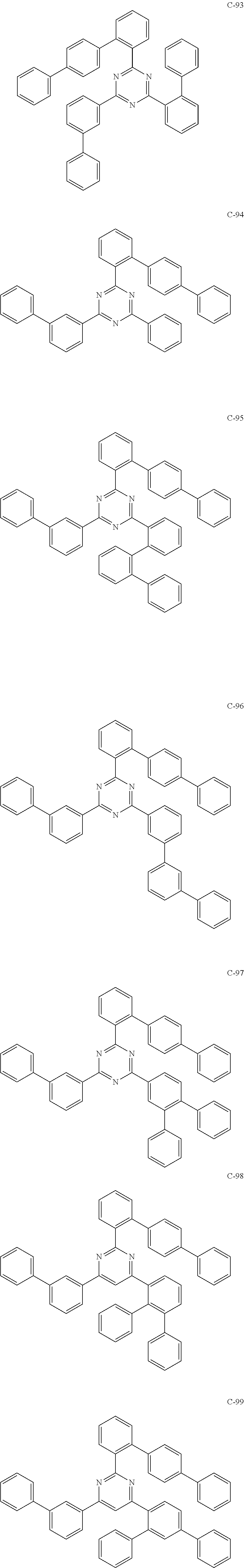 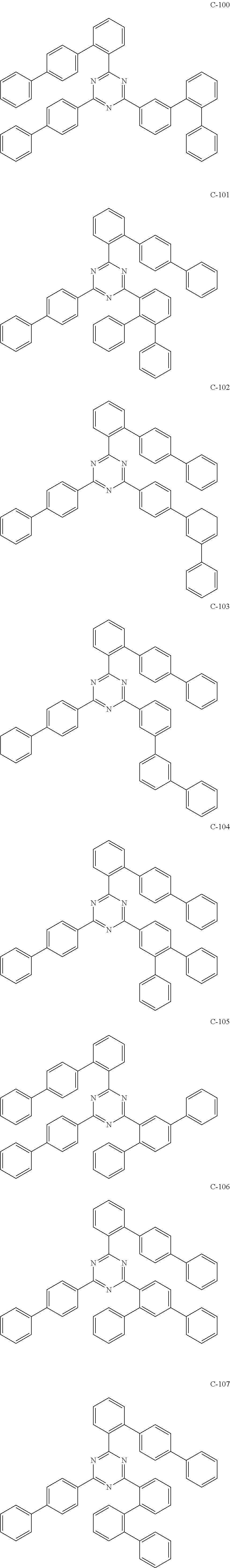 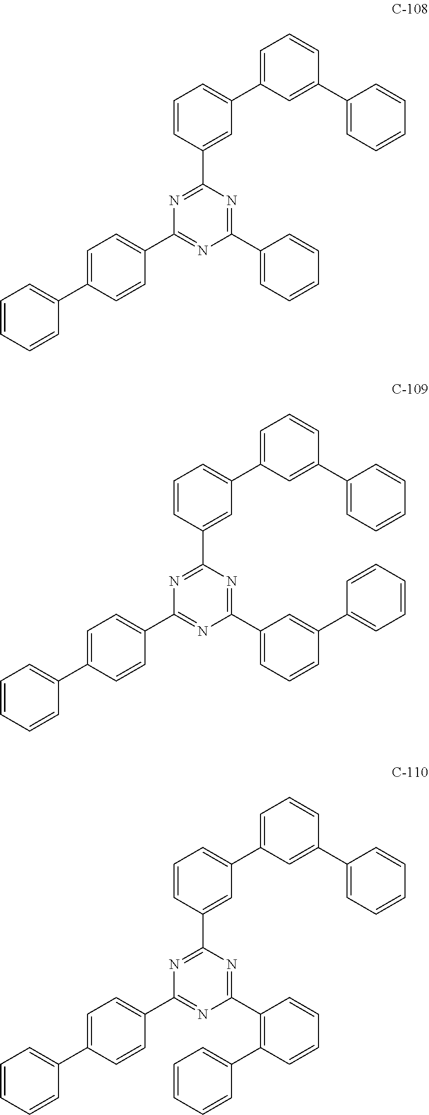 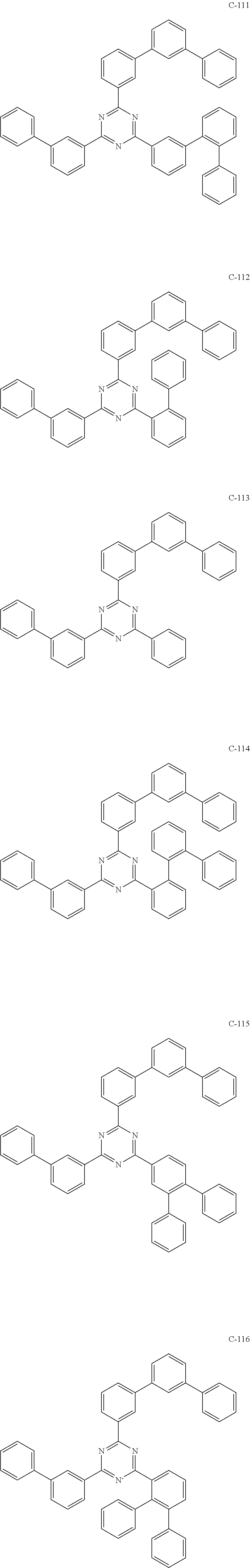 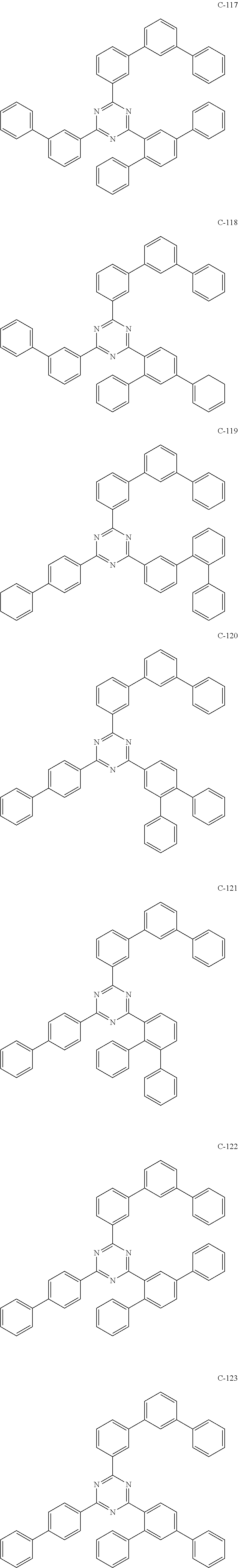 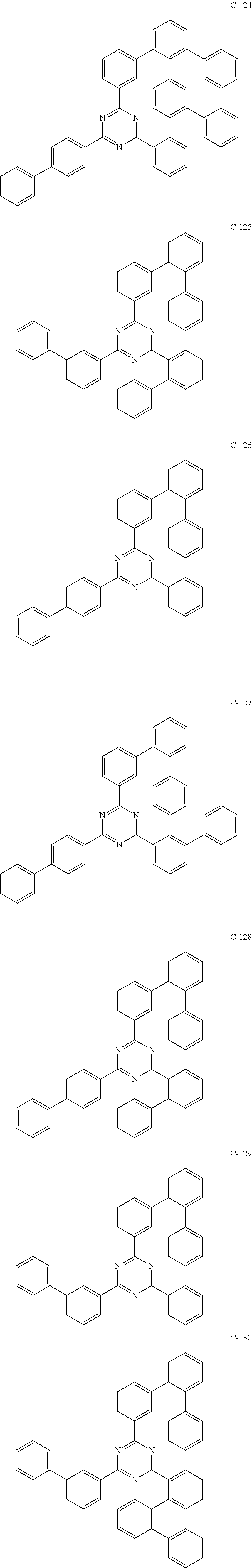 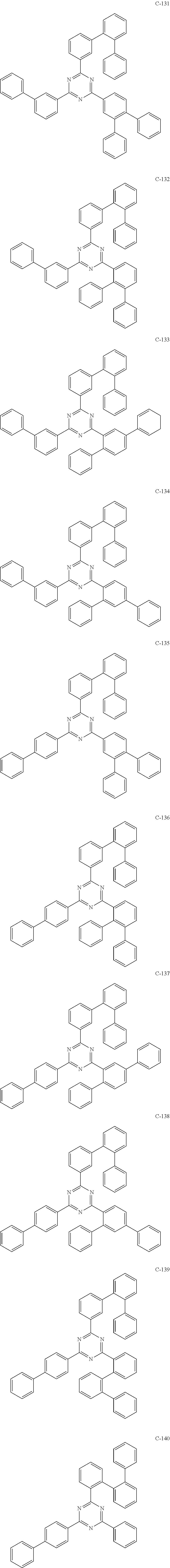 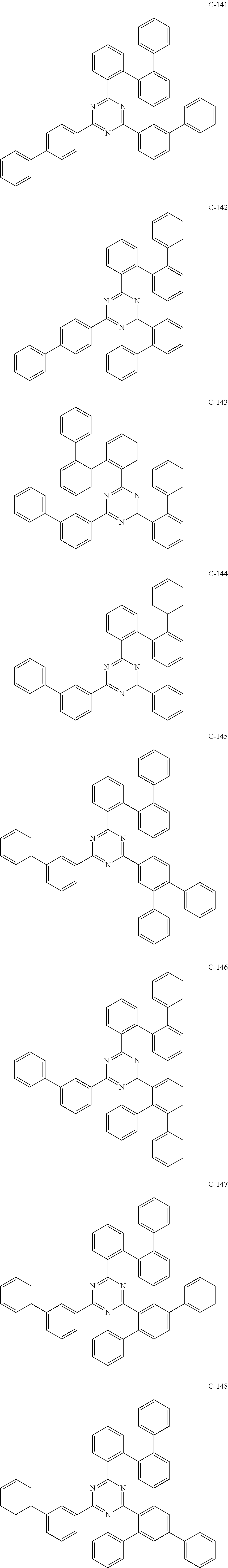 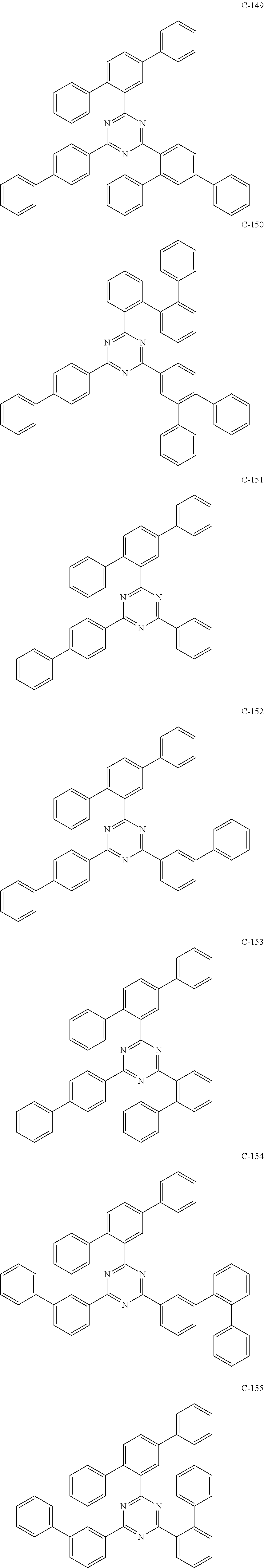 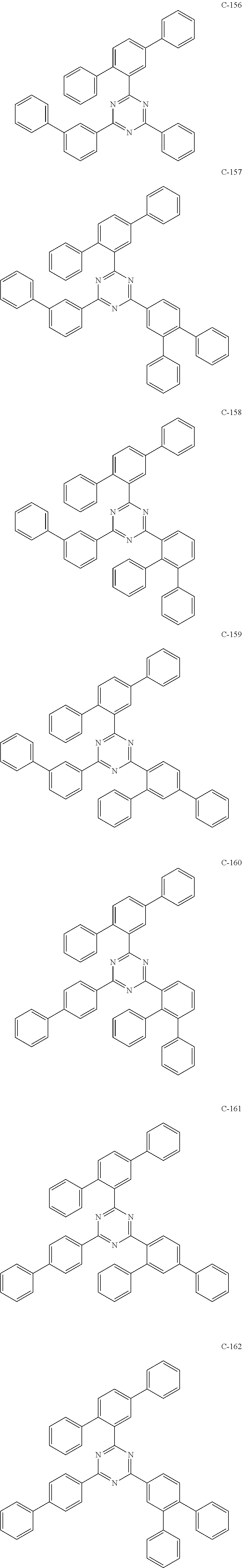 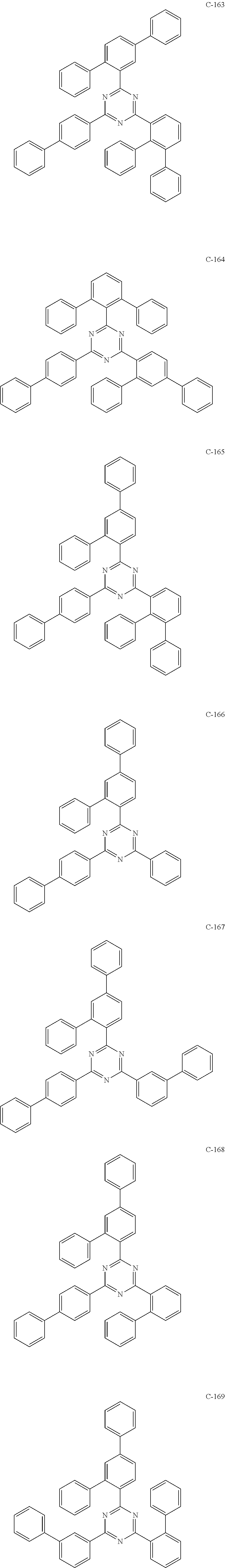 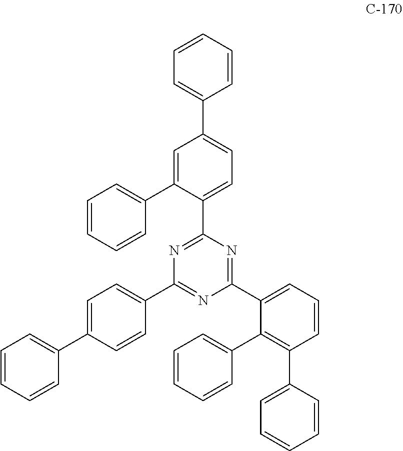 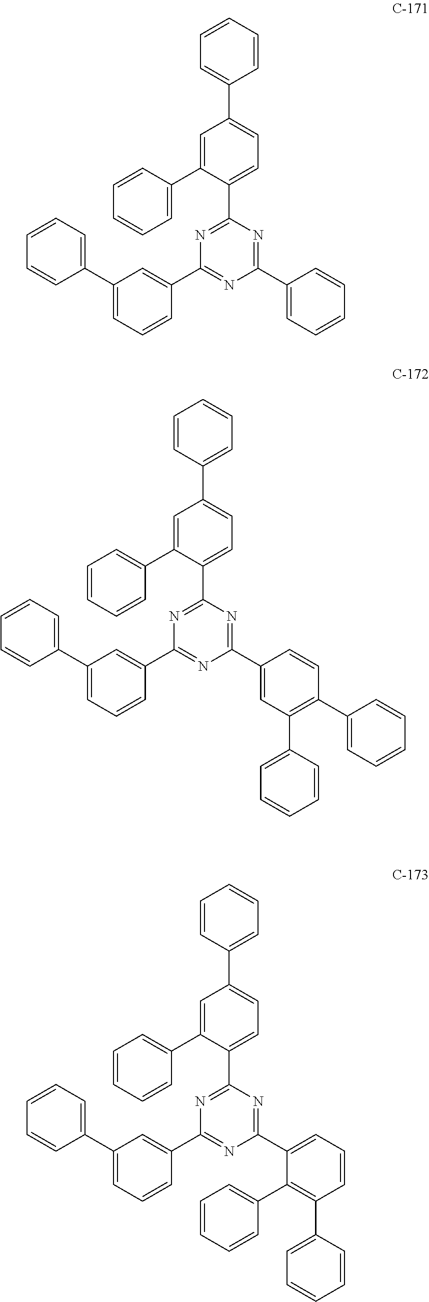 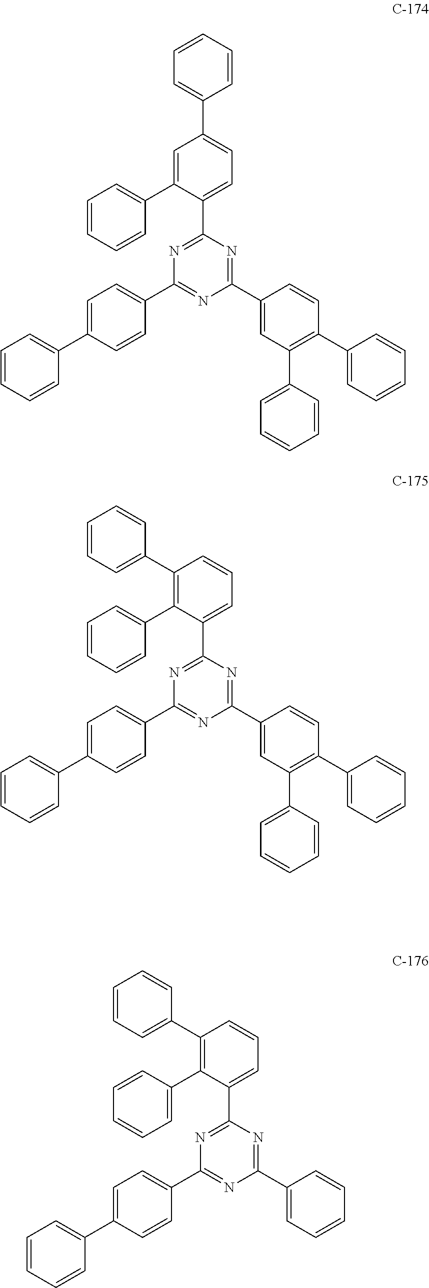 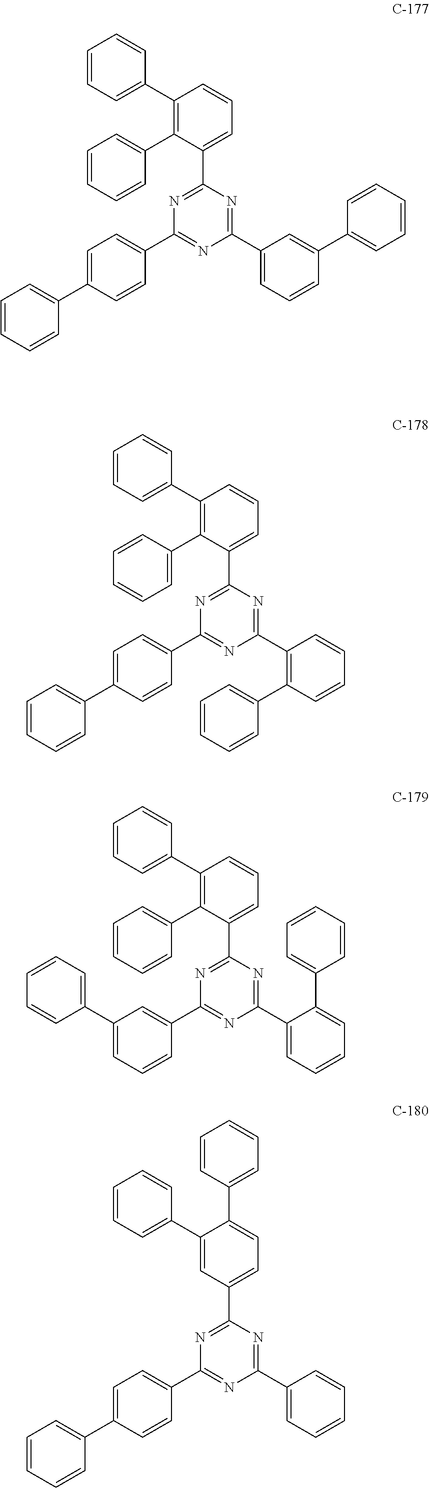 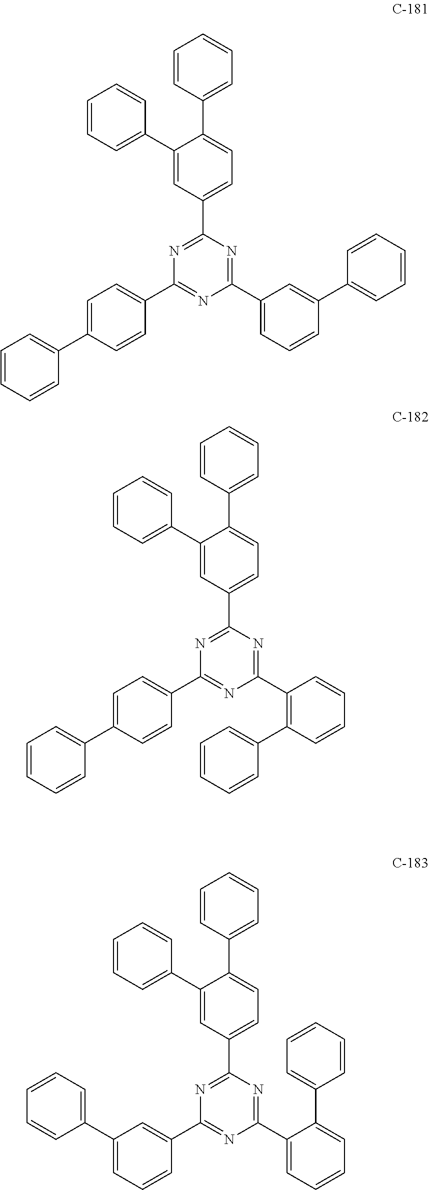 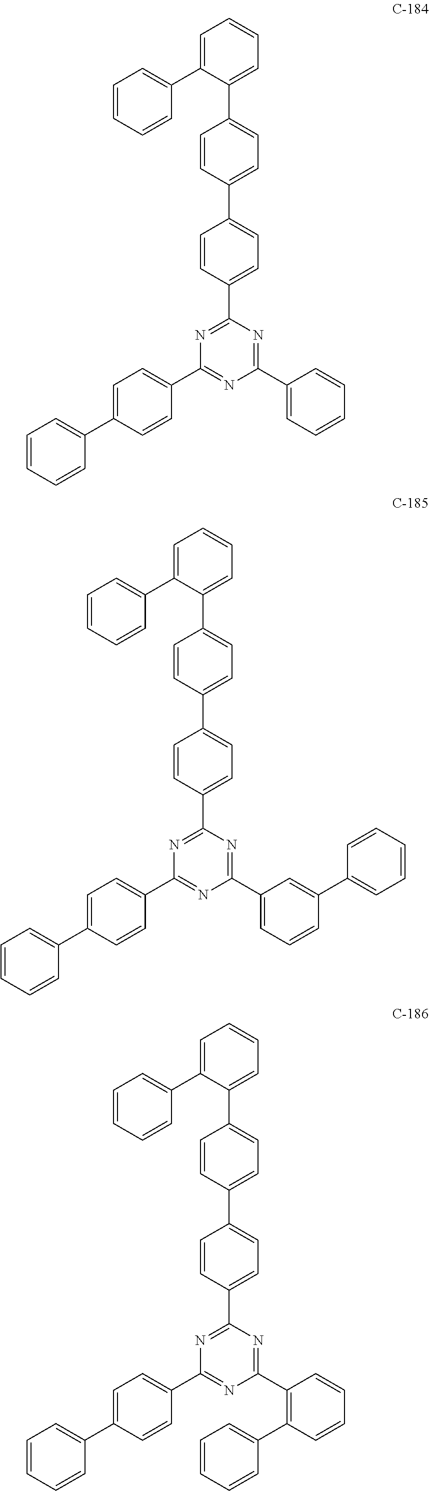 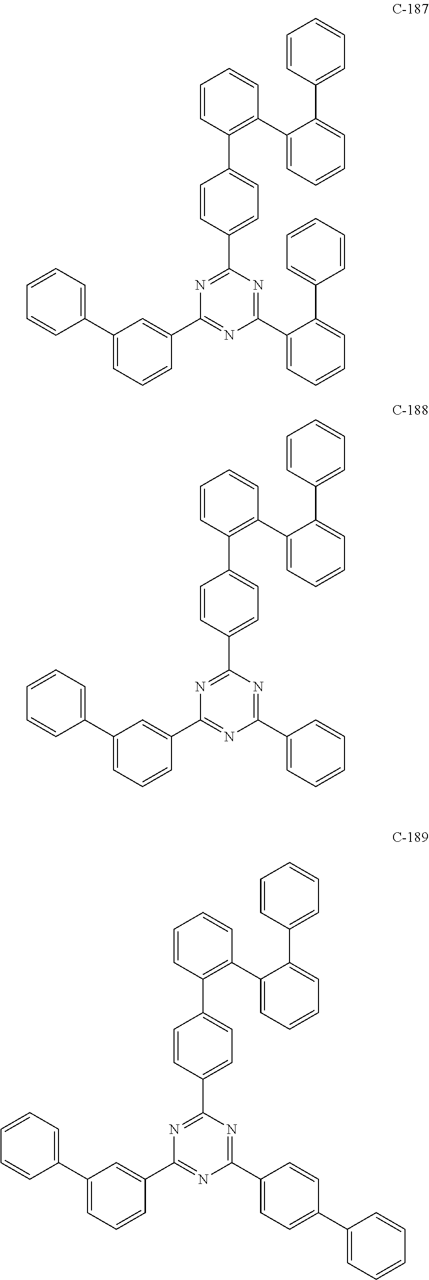 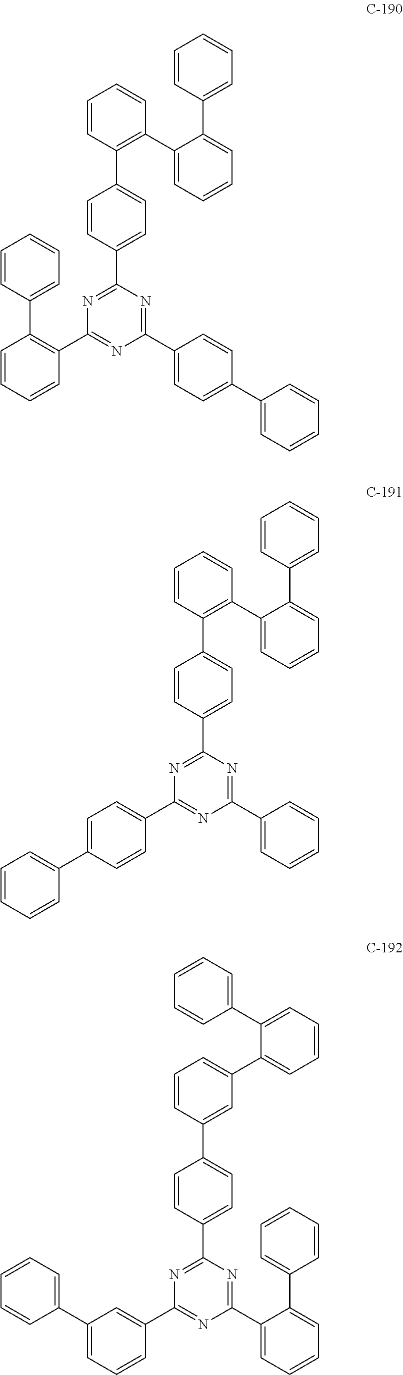 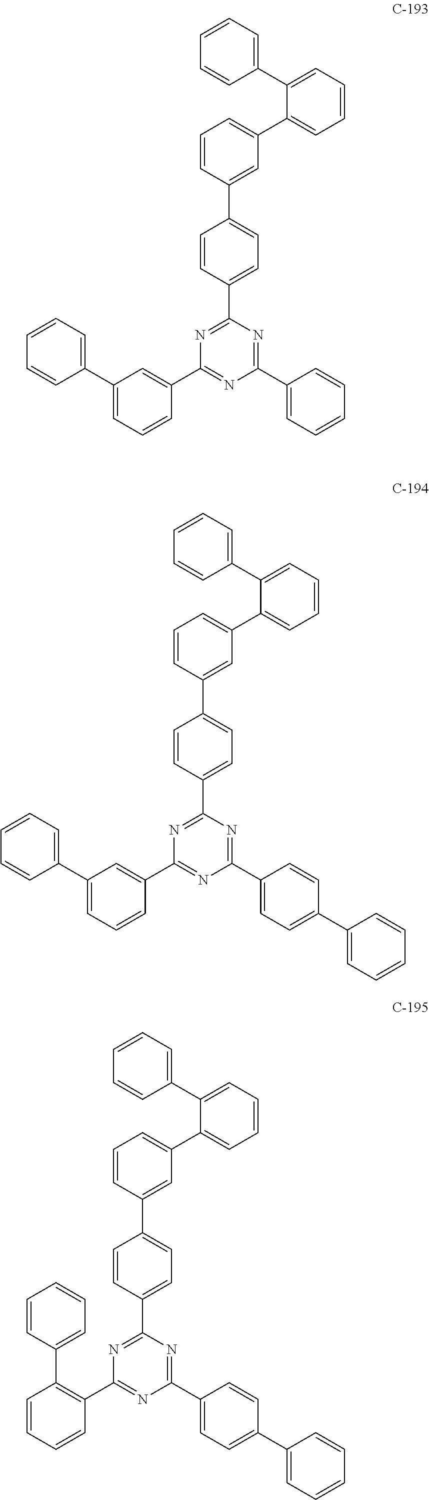 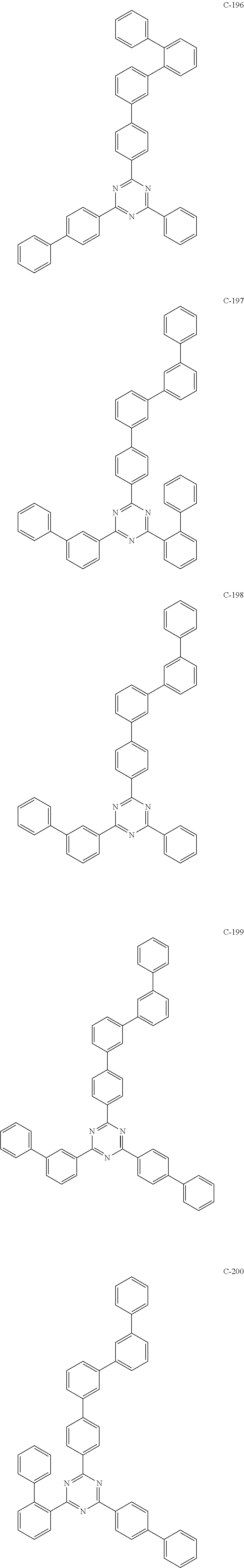 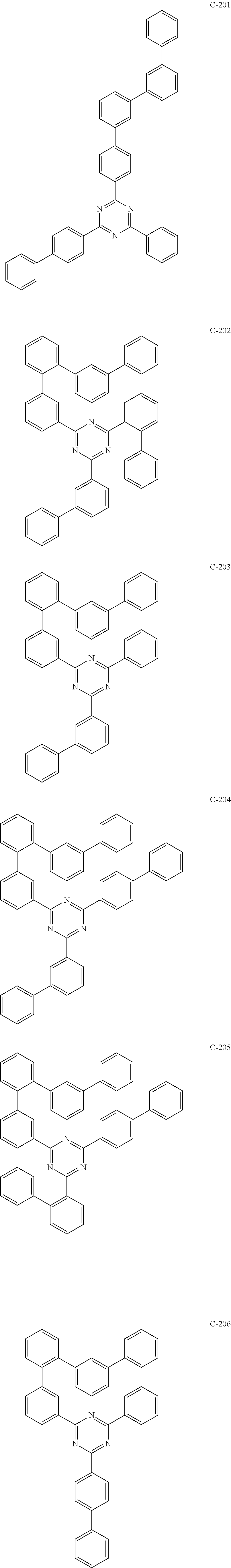 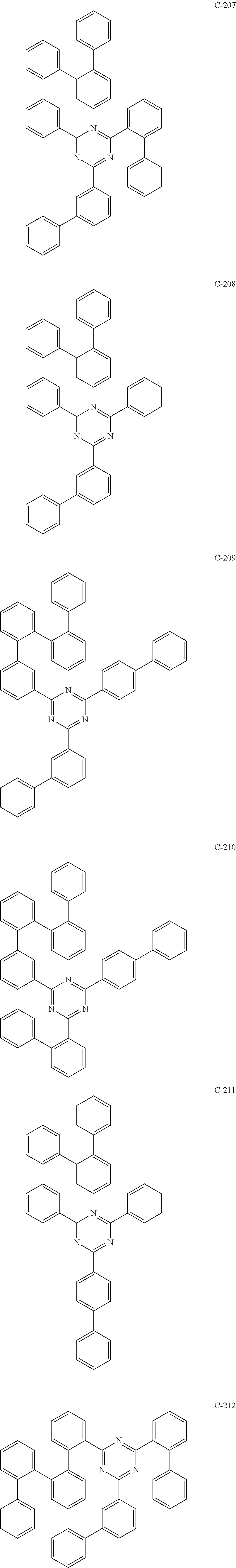 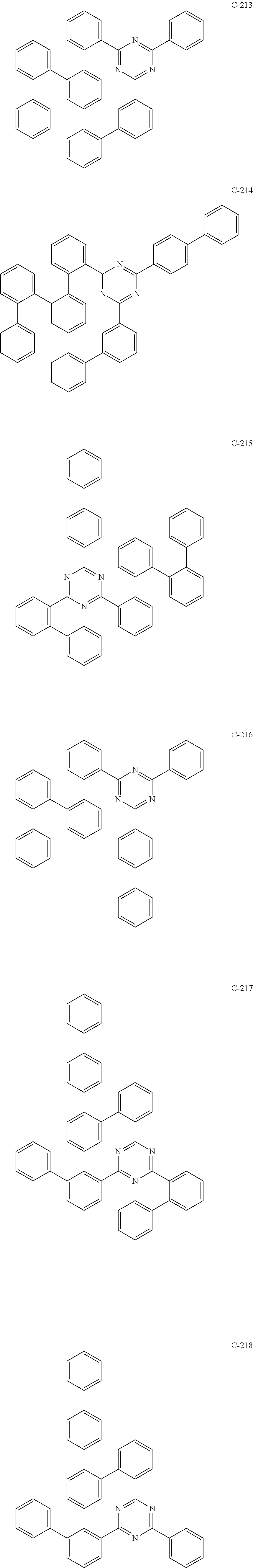 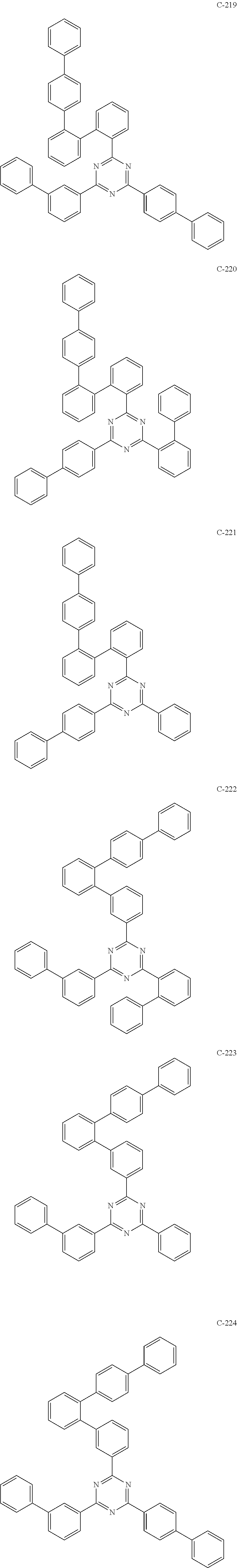 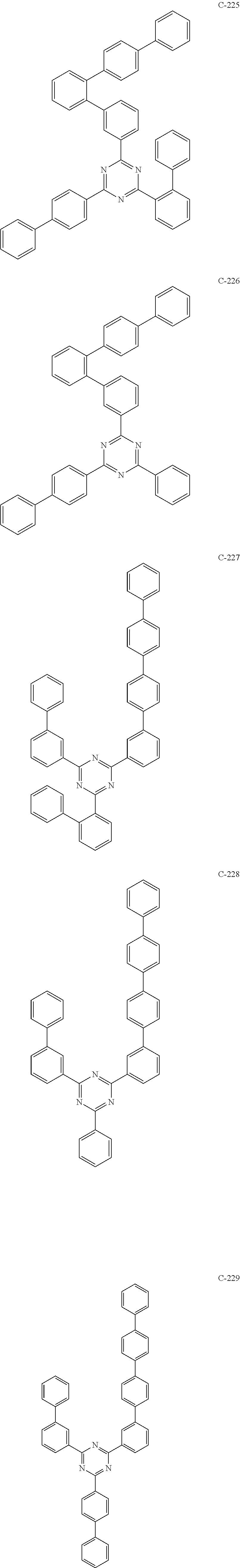 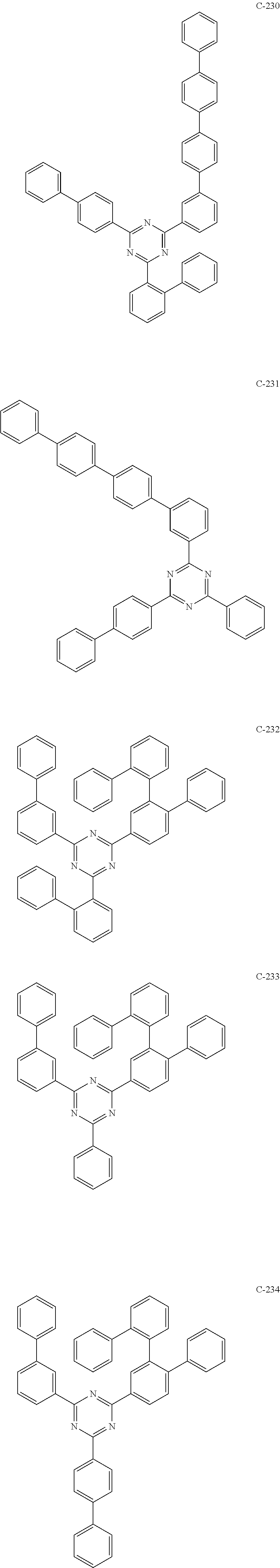 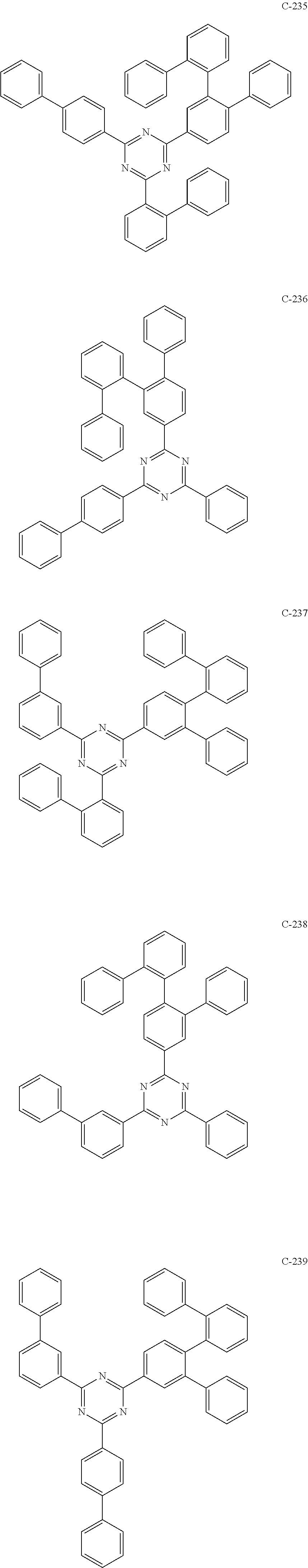 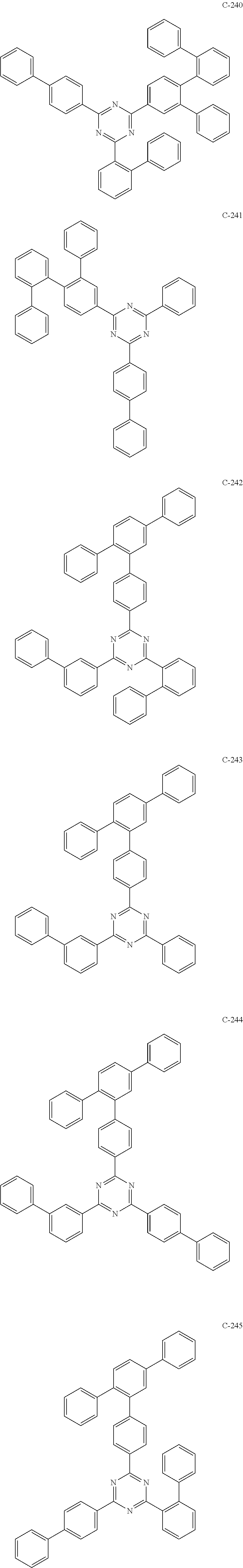 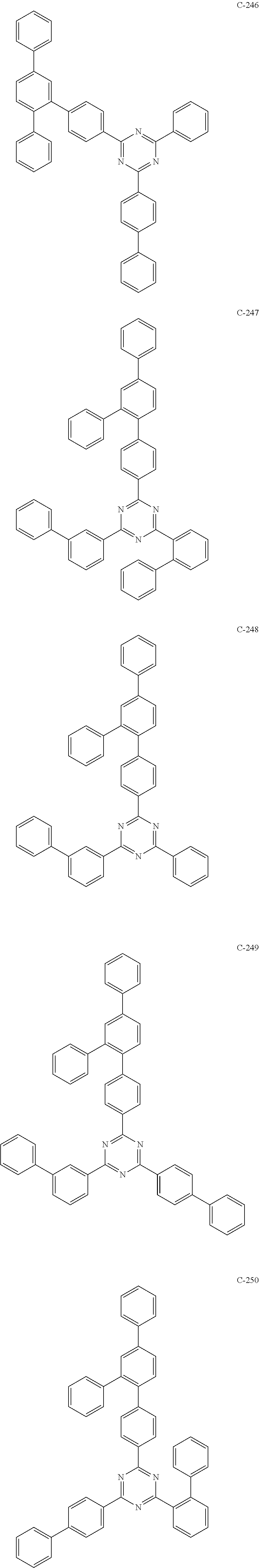 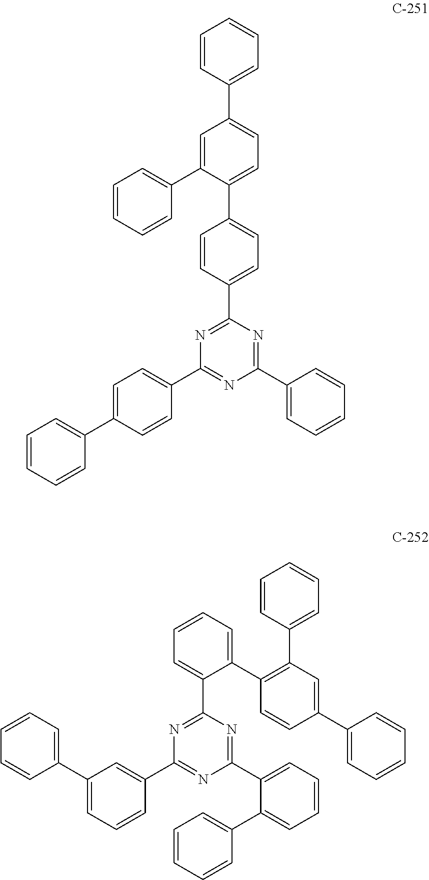 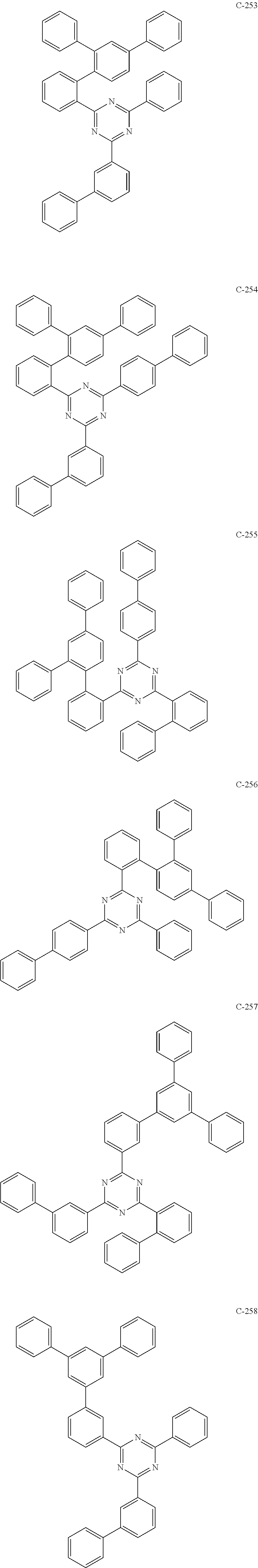 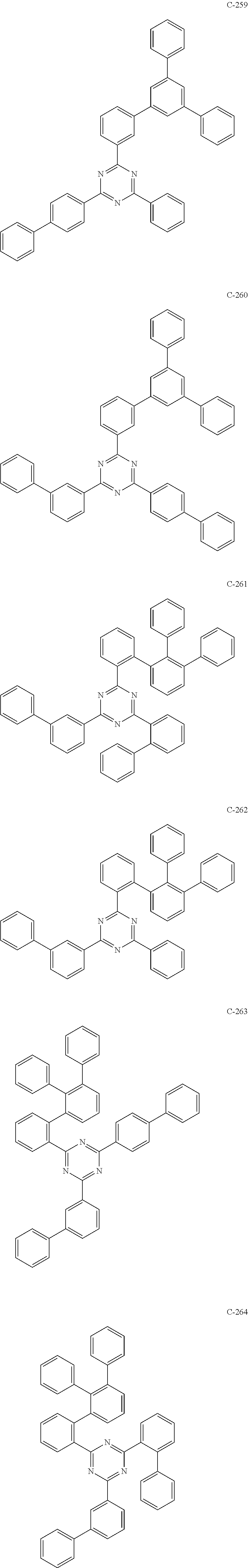 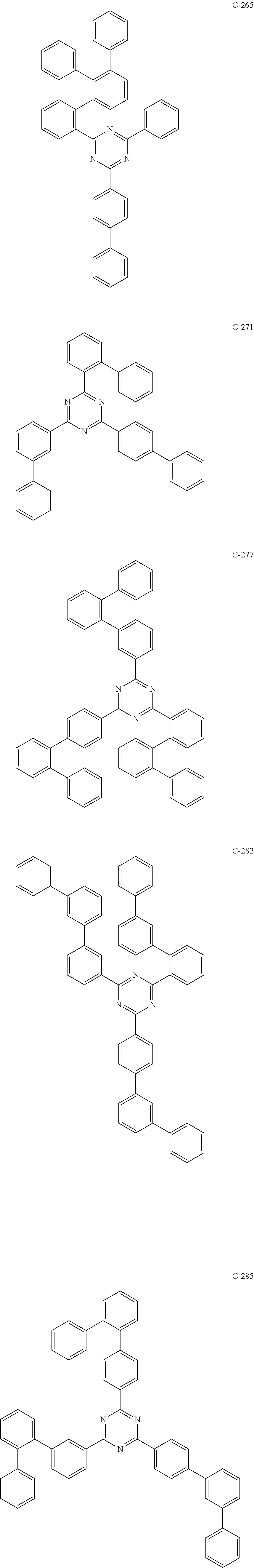 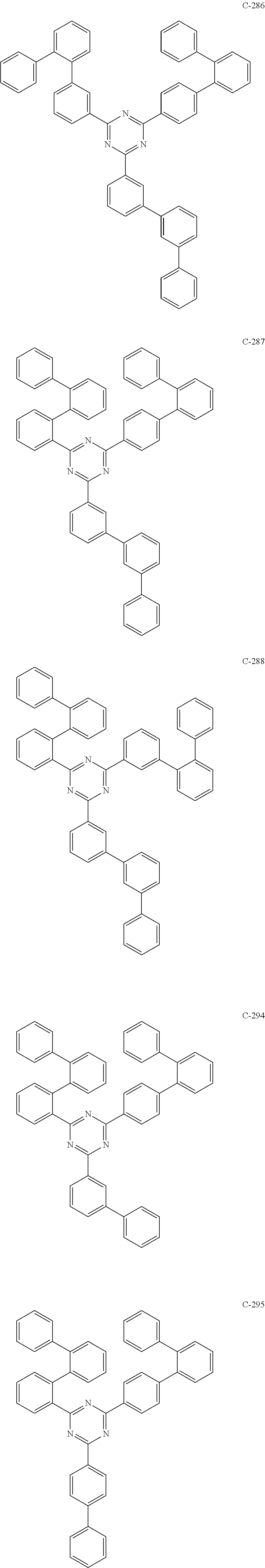 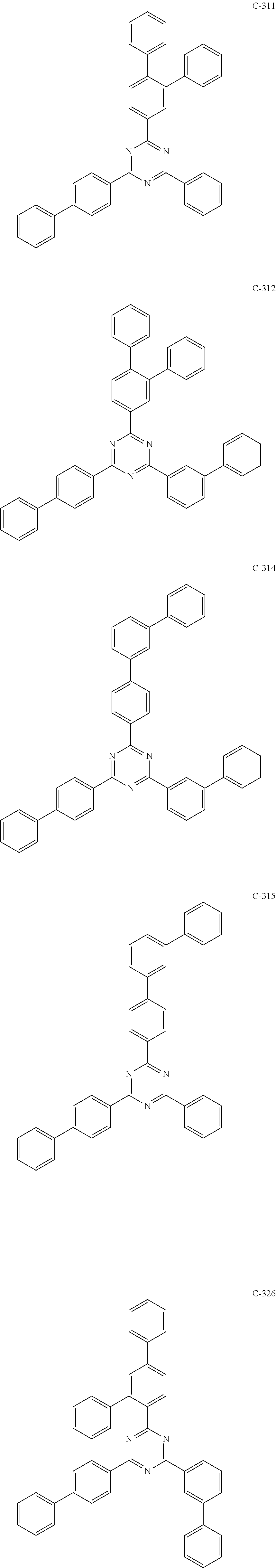 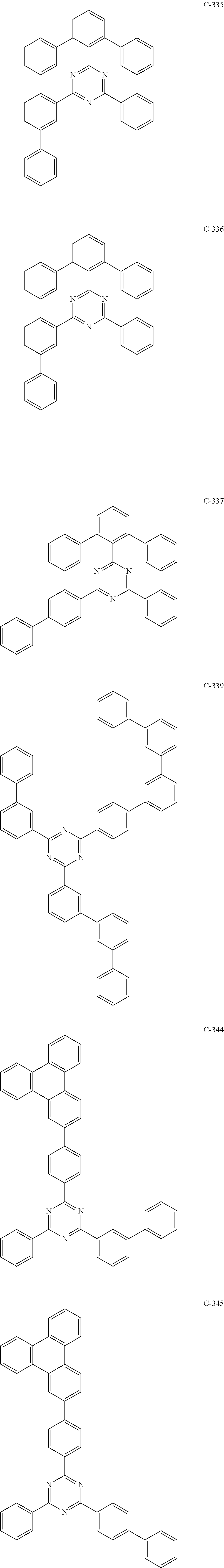 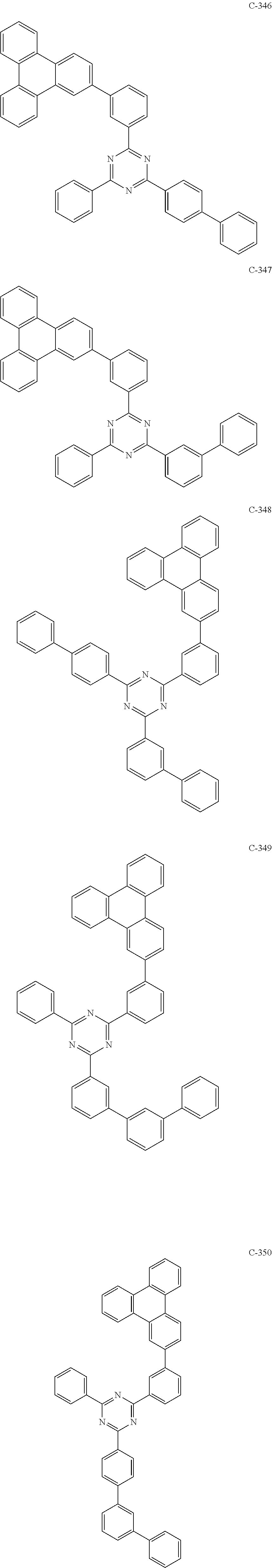 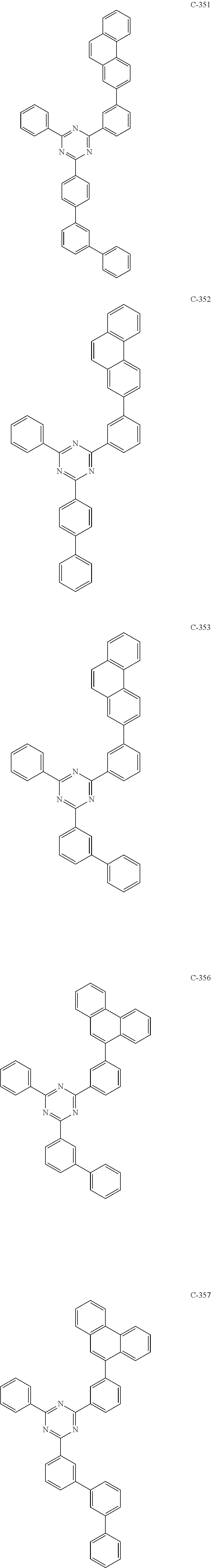 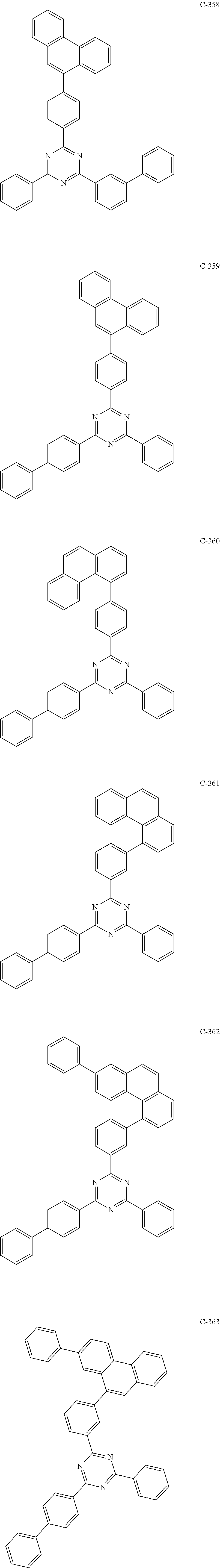 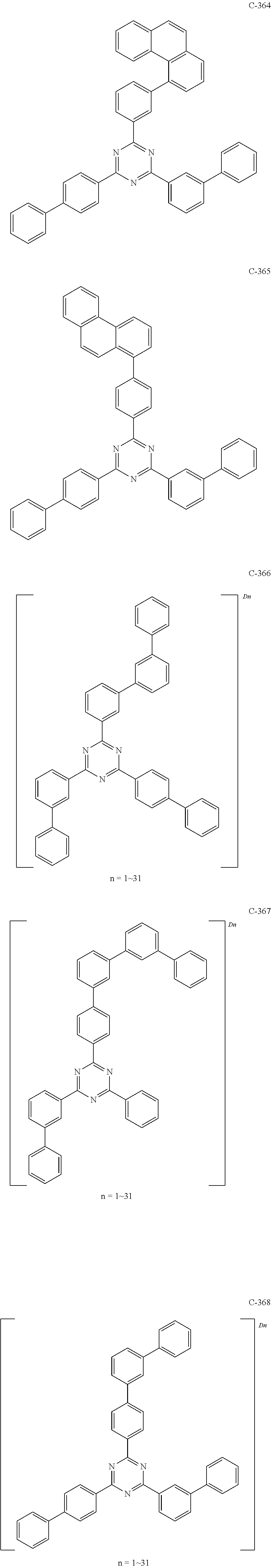 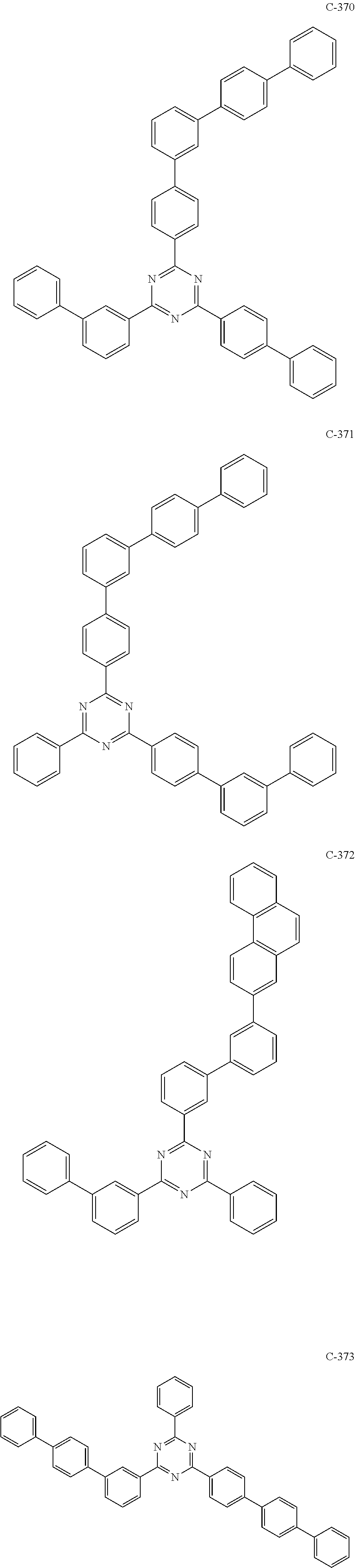 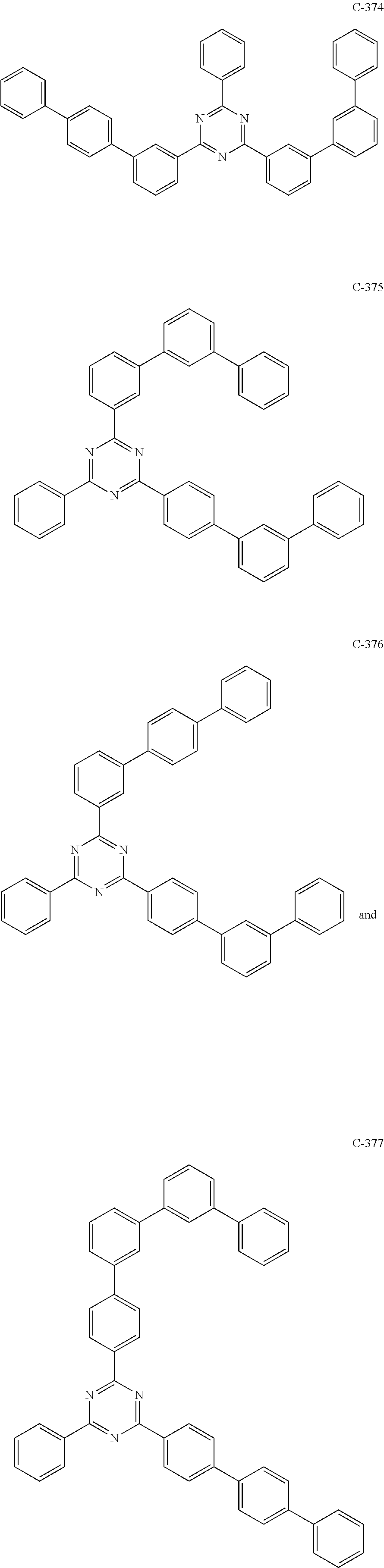
wherein Dn means that n number of hydrogen atoms are replaced with deuterium.

18. An organic electroluminescent device comprising the organic electroluminescent compound according to claim 15.
